(19) United States
(12) Patent Application Publication
(10) Pub. No.: US 2019/0151365 A1
(43) Pub. Date: May 23, 2019

(54) COMBINATION THERAPIES OF CHIMERIC ANTIGEN RECEPTORS AND PD-1 INHIBITORS

(71) Applicants: Novartis AG, Basel (CH); The Trustees of the University of Pennsylvania, Philadelphia, PA (US)

(72) Inventors: Oezlem Anak, Riedisheim (FR); Sanela Bilic, Urbandale, IA (US); Jennifer Brogdon, Sudbury, MA (US); John Scott Cameron, Belmont, MA (US); William Chou, Maplewood, NJ (US); Stephan Grupp, Havertown, PA (US); Danny Roland Howard, Jr., Washington, NJ (US); Randi Isaacs, Basking Ridge, NJ (US); Carl H. June, Merion Station, PA (US); Simon Lacey, Media, PA (US); Shannon Maude, Conshohocken, PA (US); Jan J. Melenhorst, Cherry Hill, NJ (US); Stephen Shuster, Springfield, PA (US); Alfonso Quintas-Cardama, Malvern (US)

(73) Assignees: Novartis AG, Basel (CH); The Trustees of the University of Pennsylvania, Philadelphia, PA (US); The Trustees of the University of Pennsylvania, Philadelphia, PA (US)

(21) Appl. No.: 16/321,322

(22) PCT Filed: Jul. 28, 2017
(86) PCT No.: PCT/US2017/044425

§ 371 Date: Jan. 28, 2019

Related U.S. Application Data
(60) Provisional application No. 62/368,100, filed on Jul. 28, 2016.
(60) Provisional application No. 62/455,547, filed on Feb. 6, 2017.
(60) Provisional application No. 62/482,846, filed on Apr. 7, 2017.
(60) Provisional application No. 62/514,542, filed on Jun. 2, 2017.

(51) Int. Cl. A61K 35/17; C07K 14/725; C07K 16/28; A61P 35/00; G01N 33/574

(52) U.S. Cl. CPC A61K 35/17; C07K 14/7051; C07K 16/2827; A61P 35/00; G01N 33/574; C07K 16/2818

(57) ABSTRACT

Provided are compositions and methods for treating diseases, e.g., cancers, e.g., diseases associated with expression of an antigen, e.g., CD 19, comprising administering a cell that expresses a chimeric antigen receptor (CAR) specific to the antigen, e.g., CD19, in combination with a PD-1 inhibitor.

This application claims priority to U.S. Ser. No. 62/368,100 filed Jul. 28, 2016, U.S. Ser. No. 62/455,547 filed Feb. 6, 2017, U.S. Ser. No. 62/482,846 filed Apr. 7, 2017, and U.S. Ser. No. 62/514,542 filed Jun. 2, 2017, the contents of all of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jul. 27, 2017, is named N2067-7109WO_SL.txt and is 907,582 bytes in size.

### FIELD OF THE INVENTION

The present invention relates generally to the use of cells, e.g., immune effector cells, engineered to express a Chimeric Antigen Receptor (CAR) that targets an antigen, e.g., CD19, in combination with PD-1 inhibitors to treat a disease.

### BACKGROUND OF THE INVENTION

Many patients with B cell malignancies are incurable with standard therapy. In addition, traditional treatment options often have serious side effects. Attempts have been made in cancer immunotherapy, however, several obstacles render this a very difficult goal to achieve clinical effectiveness. Although hundreds of so-called tumor antigens have been identified, these are generally derived from self and thus are poorly immunogenic. Furthermore, tumors use several mechanisms to render themselves hostile to the initiation and propagation of immune attack.

Recent developments using chimeric antigen receptor (CAR) modified autologous T cell (CART) therapy, which relies on redirecting T cells to a suitable cell-surface molecule on cancer cells such as B cell malignancies, show promising results in harnessing the power of the immune system to treat B cell malignancies and other cancers (see, e.g., Sadelain et al., Cancer Discovery 3:388-398 (2013)). The clinical results of the murine derived CART19 (i.e., “CTL019”) have shown promise in establishing complete remissions in patients suffering with CLL as well as in childhood ALL (see, e.g., Kalos et al., Sci Transl Med 3:95ra73 (2011), Porter et al., NEJM 365:725-733 (2011), Grupp et al., NEJM 368:1509-1518 (2013)). Besides the ability for the chimeric antigen receptor on the genetically modified T cells to recognize and destroy the targeted cells, a successful therapeutic T cell therapy needs to have the ability to proliferate and persist over time, in order to survey for leukemic relapse. The variable quality of T cells, resulting from anergy, suppression, or exhaustion, will have effects on CAR-transformed T cells' performance, over which skilled practitioners have limited control at this time. To be effective, CAR transformed patient T cells need to persist and maintain the ability to proliferate in response to the cognate antigen. It has been shown that ALL patient T cells perform can do this with CART19 comprising a murine scFv (see, e.g., Grupp et al., NEJM 368:1509-1518 (2013)).

### SUMMARY OF THE INVENTION

The present disclosure features, at least in part, methods and compositions for treating a disease (e.g., cancer), e.g., disease associated with an antigen, e.g., disease associated with the expression of CD19, e.g., a cancer, in a subject by using a combination therapy that includes a cell, e.g., an immune effector cell, expressing a chimeric antigen receptor (CAR) that specifically binds to an antigen, e.g., antigen described herein, e.g., CD19 (also referred to herein as a “CD19 CAR-expressing cell”) (also referred to herein as a “CAR therapy”) and an inhibitor of Programmed Death-1 (also referred to herein as a “PD-1 inhibitor”). In some embodiments, the CAR that specifically binds to the antigen, e.g., CD19, includes an antigen binding domain, e.g., a CD19 binding domain, a transmembrane domain, and an intracellular signaling domain, e.g., as described herein. In some embodiments, the PD-1 inhibitor is an antibody molecule, a polypeptide, a small molecule, or a polynucleotide, e.g., an inhibitory nucleic acid. In one embodiment, the PD-1 inhibitor is an antibody molecule, e.g., an antibody molecule described herein. Without wishing to be bound by theory, treating a subject having a disease (e.g., cancer), e.g., disease associated with CD19 expression, e.g., a cancer described herein, with a combination therapy that includes a CAR-expressing cell (e.g., CD19 CAR-expressing cell) and a PD-1 inhibitor is believed to result in improved inhibition or reduction of tumor progression in the subject, e.g., as compared to treating a subject having the disease with either a CAR-expressing cell (e.g., CD19 CAR-expressing cell) or a PD-1 inhibitor alone. For example, inhibition of the PD-1/PD-L1 interaction, in combination with the CAR therapy, can result in one or more of: (i) activation (or reactivation) of CAR-expressing cells (e.g., CD19 CAR-expressing cells); (ii) expansion in a population of CAR-expressing cells; (iii) sustained duration of a therapeutic response to a CAR therapy; (iv) increased persistence of the CAR therapy, (v) reduction of exhausted effector T cells function, (vi) reversal or relief of T cell exhaustion, (vii) increased cytokine (e.g., IL-6, or IL-2) levels; or (viii) decreased expression of checkpoint inhibitors (e.g., one or more of PD-1, TIM-3 or LAG-3) on immune effector cells (e.g., CD4+ and/or CD8+ cells, e.g., CAR-expressing immune effector cells), thus resulting in an improved therapeutic outcome in a subject treated with the combination therapy, e.g., compared to a subject receiving a CAR-therapy alone or a PD-1 inhibitor alone.

Accordingly, in one aspect, the disclosure features a method of treating a subect having a disease (e.g., cancer), e.g., a disease associated with an antigen, e.g., a disease associated with expression of CD19, e.g., a cancer as described herein. The method includes administering to the subject a cell, e.g., a population of cells, comprising, e.g., expressing a CAR that specifically binds to an antigen, e.g., CD19 (also referred to herein as a CAR therapy), and a PD-1 inhibitor. In one embodiment, the CAR-expressing cell and the PD-1 inhibitor is administered sequentially. In one embodiment, the PD-1 inhibitor is administered prior to administration of the CAR-expressing cell (e.g., CD19 CAR-expressing cell). In one embodiment, the PD-1 inhibitor is administered after the administration of the CAR-expressing cell (e.g., CD19 CAR-expressing cell). In one embodiment, the PD-1 inhibitor and CAR-expressing cell (e.g., CD19 CAR-expressing cell) are administered simultaneously or concurrently.

In embodiments, the CAR-expressing cell e.g., CD19 CAR-expressing cell described herein, and the PD-1 inhibitor are administered sequentially, e.g., in any order. In one embodiment, the combination is administered in a treatment interval. In one embodiment, the treatment interval comprises a single dose of the PD-1 inhibitor and a single dose of the CAR-expressing cell (e.g., in any order). In another embodiment, the treatment interval comprises multiple doses (e.g., a first and second dose) of the PD-1 inhibitor and a dose of the CAR-expressing cell (e.g., in any order).

In a related aspect, the disclosure provides a method of treating a subject having a cancer. The method comprises administering to the subject:
(i) a CAR therapy comprising a population of immune effector cells, comprising, e.g., expressing, a CAR, wherein the CAR comprises an antigen (e.g., a CD19) binding domain, a transmembrane domain, and an intracellular signaling domain; and
(ii) a PD-1 inhibitor.
   In some embodiments, the dose of the PD-1 inhibitor, e.g., anti-PD-1 antibody molecule, is about 200 mg to about 450 mg, e.g., about 300 mg to about 400 mg, e.g., administered every 2 weeks, 3 weeks, 4 weeks, or 5 weeks.

In another aspect, the disclosure provides a method of treating a subject having a cancer. The method comprises administering to the subject:
(i) a CAR therapy comprising a population of immune effector cells comprising, e.g., expressing, a CAR, wherein the CAR comprises an antigen (e.g., a CD19) binding domain, a transmembrane domain, and an intracellular signaling domain; and
(ii) a PD-1 inhibitor.

In some embodiments, administration of the PD-1 inhibitor is initiated 20 days or less after administration of the CAR therapy. For example, administration of the PD-1 inhibitor is initiated 16 days or less, 15 days or less, 14 days or less, 13 days or less, 12 days or less, 11 days or less, 10 days or less, 9 days or less, 8 days or less, 7 days or less, 6 days or less, 5 days or less, 4 days or less, 3 days or less, 2 days or less, after administration of the CAR therapy.

In another aspect, the disclosure provides a method of treating a subject having a cancer. The method comprises administering to the subject:
(i) a CAR therapy comprising a population of immune effector cells comprising, e.g., expressing, a CAR, wherein the CAR comprises an antigen (e.g., a CD19) binding domain, a transmembrane domain, and an intracellular signaling domain; and
(ii) a PD-1 inhibitor.

In some embodiments, administration of the PD-1 inhibitor is initiated after the subject has, or is identified as having, one or more of the following:
(a) a partial or no detectable response to the CAR therapy,
(b) a relapsed cancer after the CAR therapy,
(c) a cancer refractory to the CAR therapy;
(d) a progressive form of the cancer after the CAR therapy; or
(e) B cell recovery, e.g., less than 3 months, after the CAR therapy.

In yet another aspect, the disclosure provides a method of treating a subject having a cancer. The method comprises administering to the subject:
(i) a CAR therapy comprising a population of immune effector cells comprising, e.g., expressing, a chimeric antigen receptor (CAR), wherein the CAR comprises an antigen (e.g., a CD19) binding domain, a transmembrane domain, and an intracellular signaling domain; and
(ii) a PD-1 inhibitor.
   In some embodiments, administration of the PD-1 inhibitor is initiated after administration of the CAR therapy, and the subject does not have, or has not been identified as having, one or more of the following:
(a) a partial or no detectable response to the CAR therapy,
(b) a relapsed cancer after the CAR therapy,
(c) a cancer refractory to the CAR therapy;
(d) a progressive form of the cancer; or
(e) B cell recovery, e.g., less than 3 months, after the CAR therapy.

In another aspect, the disclosure provides a CAR therapy for use in combination with a PD-1 inhibitor in any of the methods disclosed herein. In other embodiments, disclosed herein is the use of a CAR therapy in combination with a PD-1 inhibitor in the preparation of a medicament for treating a disorder, e.g., a proliferative disorder, e.g., a cancer.

Additional features or embodiments of any of the methods, uses, compositions or combinations disclosed herein include one or more of the following:

In some embodiments, one or more, e.g., 1, 2, 3, 4, or 5 or more, subsequent doses of the PD-1 inhibitor can be administered. In one embodiment, up to 6 doses of the PD-1 inhibitor are administered.

In some embodiments, the method or use further comprises evaluating the presence or absence of CRS in the subject. In one embodiment, the subject does not have, or is identified, as not having CRS, e.g., severe CRS (e.g., CRS grade 3 or grade 4), after the CAR therapy. In other embodiments, administration of the PD-1 inhibitor is initiated after the subject is identified as not having CRS, e.g., severe CRS (e.g., CRS grade 3 or grade 4), after the CAR therapy.

In other embodiments, administration of the PD-1 inhibitor is initiated after treatment of CRS, e.g., CRS resolution, after the CAR therapy. In one embodiment, the CRS is resolved to grade 1. In an embodiment, the CRS is resolved to undetectable levels.

Where the treatment interval comprises a single dose of the PD-1 inhibitor and a single dose of the CAR-expressing cell, in certain embodiments, the dose of PD-1 inhibitor and the dose of the CAR-expressing cell are administered simultaneously or concurrently. For example, the dose of the PD-1 inhibitor and the dose of the CAR-expressing cell are administered within 20 days, 18 days, 16 days, 15 days, 12 days, 10 days, 9 days, 8 days, 7 days, 6 days, 5 days, 4 days, 3 days, 2 days, 1 day, 24 hours, 12 hours, 6 hours, 4 hours, 2 hours, or less) of each other. In embodiments, the treatment interval is initiated upon administration of the first-administered dose and completed upon administration of the later-administered dose.

Where the treatment interval comprises a single dose of the PD-1 inhibitor and a single dose of the CAR-expressing cell, in certain embodiments, the dose of the PD-1 inhibitor and the dose of the CAR-expressing cell are administered sequentially. In embodiments, the dose of the CAR-expressing cell is administered prior to the dose of the PD-1 inhibitor, and the treatment interval is initiated upon administration of the dose of the CAR-expressing cell and completed upon administration of the dose of the PD-1 inhibitor. In other embodiments, the dose of the PD-1 inhibitor is administered prior to the dose of the CAR-expressing cell, and the treatment interval is initiated upon administration of the dose of the PD-1 inhibitor and completed upon administration of the dose of the CAR-expressing cell. In one embodiment, the treatment interval further comprises one or more, e.g., 1, 2, 3, 4, or 5 or more, subsequent doses of the PD-1 inhibitor. In such embodiments, the treatment interval comprises two, three, four, five, six, or more, doses of PD-1 inhibitor and one dose of the CAR-expressing cell. In one embodiment, the dose of the CAR-expressing cell is administered at least 2 days, at least 3 days, at least 4 days, at least 5 days, at least 6 days, at least 7 days, at least 8 days, at least 9 days, at least 10 days, at least 8 days, at least 9 days, at least 10 days, at least 11 days, at least 12 days, at least 13 days, or at least 2 weeks before or after a dose of PD-1 inhibitor is administered. In embodiments where more than one dose of PD-1 inhibitor is administered, the dose of the CAR-expressing cell is administered at least 2 days, at least 3 days, at least 4 days, at least 5 days, at least 6 days, at least 7 days, at least 8 days, at least 9 days, at least 10 days, at least 8 days, at least 9 days, at least 10 days, at least 11 days, at least 12 days, at least 13 days, or at least 2 weeks before or after the first dose of PD-1 inhibitor is administered or after the initiation of the treatment interval. In one embodiment, the dose of the PD-1 inhibitor is administered about 25-40 days (e.g., about 25-30, 30-35, or 35-40 days, e.g., about 35 days) or about 2-7 weeks (e.g., 2, 3, 4, 5, 6, or 7 weeks) after the dose of the CAR-expressing cell is administered. In embodiments, where more than one dose of PD-1 inhibitor is administered, the second PD-1 inhibitor dose is administered about 15-30 days (e.g., about 15-20, 20-25, or 25-30 days, e.g., about 20 days) or about 2-5 weeks (e.g., 2, 3, 4, or 5 weeks) after the first dose of PD-1 inhibitor is administered.

Where the treatment interval comprises multiple doses (e.g., a first and second, and optionally one or more subsequent doses) of a PD-1 inhibitor and a dose of a CAR-expressing cell, in certain embodiments, the dose of the CAR-expressing cell and the first dose of the PD-1 inhibitor are administered simultaneously or concurrently, e.g., within 2 days (e.g., within 2 days, 1 day, 24 hours, 12 hours, 6 hours, 4 hours, 2 hours, or less) of each other. In embodiments, the second dose of the PD-1 inhibitor is administered after either (i) the dose of the CAR-expressing cell or (ii) the first dose of the PD-1 inhibitor, whichever is later. In embodiments, the second dose of the PD-1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after (i) or (ii). In embodiments, a subsequent dose (e.g., third, fourth, or fifth dose, and so on) of the PD-1 inhibitor is administered after the second dose of the PD-1 inhibitor. In embodiments, the subsequent dose of the PD-1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after the second dose of the PD-1 inhibitor. In such embodiments, the treatment interval is initiated upon administration of the first-administered dose and completed upon administration of the second dose (or subsequent dose) of the PD-1 inhibitor.

In other embodiments where the treatment interval comprises multiple doses (e.g., a first and second, and optionally a subsequent dose) of a PD-1 inhibitor and a dose of a CAR-expressing cell, the dose of the CAR-expressing cell and the first dose of the PD-1 inhibitor are administered sequentially. In embodiments, the dose of the CAR-expressing cell is administered after administration of the first dose of the PD-1 inhibitor but before the administration of the second dose of the PD-1 inhibitor. In embodiments, a subsequent dose (e.g., third, fourth, or fifth dose, and so on) of the PD-1 inhibitor is administered after the second dose of the PD-1 inhibitor. In such embodiments, the treatment interval is initiated upon administration of the first dose of the PD-1 inhibitor and completed upon administration of the second dose (or subsequent dose) of the PD-1 inhibitor. In one embodiment, the second dose of the PD-1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after administration of the first dose of the PD-1 inhibitor. In an embodiment, where the PD-1 inhibitor is an inhibitory RNA, e.g., siRNA, the second dose is administered every 2 days to every 2 weeks. In an embodiment, where the PD-1 inhibitor is an antibody molecule, the second dose is administered every 2-3 weeks. In one embodiment, the subsequent dose (e.g., third, fourth, or fifth dose, and so on) of the PD-1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after the second dose of the PD-1 inhibitor. In one embodiment, the dose of the CAR-expressing cell is administered at least 2 days (e.g., at least 2 days, 3 days, 4 days, 5 days, 6 days, 7 days, 1 week, 2 weeks, 3 weeks, 4 weeks, 5 weeks, or more) after administration of the first dose of the PD-1 inhibitor. In one embodiment, the second dose of the PD-1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after administration of the dose of the CAR-expressing cell. In embodiments, the PD-1 inhibitor (e.g., an anti-PD-1 antibody molecule) is administered every 2-3 weeks (e.g., every 2 weeks or every 3 weeks) during the treatment interval.

In other embodiments, the dose of the CAR-expressing cell is administered before administration of the first dose of the PD-1 inhibitor. In such embodiments, the treatment interval is initiated upon administration of the CAR-expressing cell and completed upon administration of the first dose (or subsequent dose) of the PD-1 inhibitor. In embodiments, the first dose of the PD-1 inhibitor is administered at least 2 days (e.g., at least 2 days, at least 3 days, at least 4 days, at least 5 days, at least 6 days, at least 1 week, at least 8 days, at least 9 days, at least 10 days, at least 11 days, at least 12 days, at least 13 days, at least 2 weeks, at least 15 days, at least 16 days, at least 17 days, at least 18 days, at least 19 days, at least 20 days, at least 3 weeks, at least 4 weeks, at least 5 weeks, or more) after administration of the CAR-expressing cell. In some embodiments, administration of the first dose of the PD-1 inhibitor occurs about 5 to about 10 days, e.g., about 8 days, after administration of the CAR-expressing cell. In other embodiments, administration of the first dose of the PD-1 inhibitor occurs about 10 to about 20 days, e.g., about 15 or 16 days, after administration of the CAR-expressing cell. In embodiments, the second dose of the PD-1 inhibitor is administered at least 2 days (e.g., at least 2 days, at least 3 days, at least 4 days, at least 5 days, at least 6 days, at least 1 week, at least 8 days, at least 9 days, at least 10 days, at least 11 days, at least 12 days, at least 13 days, 2 weeks, at least 15 days, at least 16 days, at least 17 days, at least 18 days, at least 19 days, at least 20 days, 3 weeks, 4 weeks, 5 weeks, or more) after administration of the first dose of the PD-1 inhibitor. In embodiments, the second dose of the PD-1 inhibitor is administered at about 2-4 weeks, e.g., 3 weeks after the first dose of the PD-1 inhibitor. In embodiments, the subsequent dose (e.g., third, fourth, or fifth dose, and so on) of the PD-1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after the second dose of the PD-1 inhibitor. In embodiments, the subsequent dose (e.g., third, fourth, or fifth dose, and so on) of the PD-1 inhibitor is administered at about 2-4 weeks, e.g., 3 weeks after the previous dose of the PD-1 inhibitor. In embodiments, the first dose of the PD1 inhibitor is administered at least 2 days (e.g., at least 2 days, 3 days, 4 days, 5 days, 6 days, 7 days, 1 week, 2 weeks, 3 weeks, 4 weeks, 5 weeks, or more) after administration of the CAR-expressing cell.

In some embodiments, the treatment interval comprises one, two or three doses (e.g., a first and second, and a third dose) of a PD-1 inhibitor and a dose of a CAR-expressing cell. In one embodiment, the dose of the CAR-expressing cell and the first dose of the PD-1 inhibitor are administered sequentially. For example, the subject, e.g., a patient, receives one, two or three doses of the PD-1 inhibitor starting post administration of a CAR-expressing cell, e.g., about one week to 4 months, e.g., about 14 days to 2 months, after administration of a dose of CAR-expressing cells.

In one embodiment, any of the treatment intervals described herein can be repeated one or more times, e.g., 1, 2, 3, 4, or 5 more times. In one embodiment, the treatment interval is repeated once, resulting in a treatment regimen comprising two treatment intervals. In an embodiment, the repeated treatment interval is administered at least 1 day, e.g., at least 1 day, at least 2 days, at least 3 days, at least 4 days, at least 5 days, at least 6 days, at least 7 days, at least 2 weeks, at least 1 month, at least 3 months, at least 6 months, at least 1 year or more after the completion of the first or previous treatment interval. In an embodiment, the repeated treatment interval is administered at least 3 days after the completion of the first or previous treatment interval.

In one embodiment, any of the treatment intervals described herein can be followed by one or more, e.g., 1, 2, 3, 4, or 5, subsequent treatment intervals. The one or more subsequent treatment interval is different from the first or previous treatment interval. By way of example, a first treatment interval consisting of a single dose of a PD-1 inhibitor and a single dose of a CAR-expressing cell is followed by a second treatment interval consisting of multiple doses (e.g., two, three, four, or more doses) of a PD-1 inhibitor and a single dose of a CAR-expressing cell. In one embodiment, the one or more subsequent treatment intervals is administered at least 1 day, e.g., 1 day, 2 days, 3 days, 4 days, 5 days, 6 days, 7 days, or 2 weeks, after the completion of the first or previous treatment interval.

In any of the methods described herein, one or more subsequent doses, e.g., 1, 2, 3, 4, or 5 or more doses, of the PD-1 inhibitor is administered after the completion of one or more treatment intervals. In embodiments where the treatment intervals are repeated or two or more treatment intervals are administered, one or more subsequent doses, e.g., 1, 2, 3, 4, or 5 or more doses, of the PD-1 inhibitor is administered after the completion of one treatment interval and before the initiation of another treatment interval. In one embodiment, a dose of the PD-1 inhibitor is administered every 5 days, 7 days, 2 weeks, 3 weeks, or 4 weeks after the completion of one or more, or each, treatment intervals.

In any of the methods described herein, one or more, e.g., 1, 2, 3, 4, or 5 or more, subsequent doses of the CAR-expressing cell are administered after the completion of one or more treatment intervals. In embodiments where the treatment intervals are repeated or two or more treatment intervals are administered, one or more subsequent doses, e.g., 1, 2, 3, 4, or 5, or more doses, of the CAR-expressing cell is administered after the completion of one treatment interval and before the initiation of another treatment interval. In one embodiment, a dose of the CAR-expressing cell is administered every 2 days, 3 days, 4 days, 5 days, 7 days, 2 weeks, 3 weeks, or 4 weeks after the completion of one or more, or each, treatment intervals.

In one embodiment, the treatment interval comprises a single dose of a CAR-expressing cell that is administered prior to a first dose of a PD-1 inhibitor. In this embodiment, the first dose of the PD-1 inhibitor is administered about 7, about 8, about 9, about 10, about 11, about 12, about 13, about 14, about 15, about 16, about 17, about 18, about 19, about 20, about 25, about 30, or about 35 days after administration of the CAR-expressing cell. In embodiments, a second dose of the PD-1 inhibitor is administered after administration of the first dose of the PD-1 inhibitor. In embodiments, the second dose of the PD-1 inhibitor is administered about 20 days after administration of the first dose of the PD-1 inhibitor, e.g., about 2-4 weeks, e.g., 3 weeks after the first dose of the PD-1 inhibitor. In embodiments, subsequent doses of the PD-1 inhibitor are administered after the second dose of the PD-1 inhibitor, e.g., every 1 week, 2 weeks, 3 weeks, 4 weeks, 5 weeks, 5 days, 7 days, 10 days, 14 days, 20 days, 25 days, 30 days, or 35 days, e.g., about 2-4 weeks, e.g., 3 weeks after the previous dose of the PD-1 inhibitor.

In an embodiment, the method comprises administering a lymphodepleting chemotherapy to the subject, e.g., prior to administration of the CAR-expressing cell. In embodiments, the lymphodepleting chemotherapy comprises cyclophosphamide, e.g., hyperfractionated cyclophosphamide, e.g., at a dose of about 200-400 mg/m², e.g., about 300 mg/m², e.g., for 1-10 doses (e.g., 1, 2, 3, 4, 5, 6, 7, 8, 9, or 10 doses). In embodiments, the method comprises administering a treatment interval comprising a dose of CAR-expressing cells and multiple doses of a PD-1 inhibitor. In embodiments, the treatment interval comprises a single dose of a CAR-expressing cell (e.g., CD19 CAR-expressing cell) that is administered prior to a first dose of a PD-1 inhibitor, e.g., at least 2 weeks (e.g., 2, 3, 4, 5, 6 weeks or more) prior to the first dose of the PD-1 inhibitor (e.g., about 7, about 8, about 9, about 10, about 11, about 12, about 13, about 14, about 15, about 16, or more days prior to the first dose of the PD-1 inhibitor). In embodiments, the dose of the CAR-expressing cell is administered about 3-4 weeks before the first dose of the PD-1 inhibitor. In embodiments, the PD-1 inhibitor is administered every 2-4 weeks (e.g., every 2-3 weeks or 3-4 weeks, e.g., every 3 weeks) during the treatment interval). In embodiments, the PD-1 inhibitor is administered at a dose of about 1-3 mg/kg, e.g., about 2 mg/kg. In embodiments, the CAR-expressing cell is administered at a dose of about 1-10×10⁶ cells/kg, e.g., about 5×10⁶ cells/kg, e.g., about 5.3×10⁶ cells/kg. In embodiments, the CAR-expressing cell is administered at a dose of about 1-10×10⁸ cells per infusion, e.g., about 5×10⁸ cells per infusion.

In any of the methods described herein, the subject is administered a single dose of a CAR-expressing cell and a single dose of a PD-1 inhibitor. In one embodiment, the single dose of the CAR-expressing cell is administered at least 2 days, e.g., 2, 3, 4, 5, 6, 7, 8, 9, 10, 14, 15, 16, 17, 18, 20, 25, 30, 35, 40 days, or 2 weeks, 3 weeks, 4 weeks, or more, before administration of the single dose of the PD-1 inhibitor. In embodiments, the single dose of the CAR-expressing cell is administered about 35 days before administration of the PD-1 inhibitor.

In one embodiment, one or more, e.g., 1, 2, 3, 4, or 5, subsequent doses of a CAR-expressing cell are administered to the subject after the initial dose of the CAR-expressing cell. In one embodiment, the one or more subsequent doses of the CAR-expressing cell are administered at least 2 days, e.g., 2, 3, 4, 5, 6, 7, 8, 9, 10, 14, 20, 25, 30, 35, 40 days, or 2 weeks, 3 weeks, 4 weeks, or more, after the previous dose of the CAR-expressing cell. In one embodiment, the one or more subsequent doses of the CAR-expressing cell are administered at least 1 month, e.g., 1, 2, 3, 4, 5, 6, 7, 8, 9, 10, 11, 12, 13, 14, 15, 16, 17, 18, 19, 20 or more months, after the previous dose of the CAR-expressing cell. In one embodiment, the one or more subsequent doses of the CAR-expressing cell are administered at least 5 days after the previous dose of the CAR-expressing cell. In one embodiment, the subject is administered three doses of the CAR-expressing cell per week or one dose every 2 days.

In one embodiment, one or more, e.g., 1, 2, 3, 4, or 5, subsequent doses of PD-1 inhibitor are administered after administration of the single dose of the PD-1 inhibitor. In one embodiment, the one or more subsequent doses of the PD-1 inhibitor are administered at least 5 days, 7 days, 10 days, 14 days, 20 days, 25 days, 30 days, 2 weeks, 3 weeks, 4 weeks, or 5 weeks, e.g., 3 weeks, after the previous dose of PD-1 inhibitor.

In one embodiment, the one or more subsequent doses of the PD-1 inhibitor are administered at least 1, 2, 3, 4, 5, 6, or 7 days, after a dose of the CAR-expressing cell, e.g., the initial dose of the CAR-expressing cell.

In one embodiment, one or more, e.g., 1, 2, 3, 4, or 5, doses of the PD-1 inhibitor is administered prior to the first dose of the CAR-expressing cell.

In one embodiment, one or more, e.g., 1, 2, 3, 4, 5, or 6, doses of the PD-1 inhibitor is administered afer the first dose of the CAR-expressing cell, e.g., 2 days, 3 days, 4 days, 5 days, 6 days, 7 days, 2 weeks, 3 weeks, 4 weeks, 5 weeks, 6 weeks, 7 weeks, or 8 weeks after the first dose of the CAR-expressing cell. In one embodiment, the one or more, e.g., 1, 2, 3, 4, or 5, doses of the PD-1 inhibitor is administered after the first dose of the CAR-expresisng cells, e.g., 2, 3, 4, 5, 6, 7, 8, 9, 10, 11, 12, 13, 14, 15, 16, 17, 18, 19, or 20 months after the first dose of the CAR-expressing cell.

In one embodiment, one or more, e.g., 1, 2, 3, 4, 5, or 6, doses of the PD-1 inhibitor which is administered after the first dose of the CAR-expressing cell, is administered every 2-3 weeks, e.g., every 2, 3, 4, or 5 weeks, for at least 1 month, e.g., for 1, 2, 3, 4, 5, 6, 7, 8, 9, 10, 11, or 12 months or more. In one embodiment, the one or more doses of the PD-1 inhibitor are administered, e.g., about 2-4 weeks, e.g., 3 weeks after the previous dose of the PD-1 inhibitor, e.g., for up to six doses.

In one embodiment, the administration of the one or more doses of the CAR-expressing cell and the one or more doses of PD-1 inhibitor is repeated, e.g., 1, 2, 3, 4, or 5 more times.

In any of the methods described herein, in embodiments, the subject is further administered a chemotherapy, e.g., a chemotherapy described herein. In embodiments, the chemotherapy is administered before administration of the CAR-expressing cell. In embodiments, the chemotherapy is administered about 1-10 days (e.g., about 1-4, 1-5, 4-8, 4-10, or 5-10 days) before administration of the CAR-expressing cell.

Dosages and therapeutic regimens of the therapeutic agents disclosed herein can be determined by a skilled artisan.

In any of the administration regimens or treatment intervals described herein, in some embodiments, a dose of CAR-expressing cells (e.g., CD19 CAR-expressing cells) comprises about 10⁴ to about 10⁹ cells/kg, e.g., about 10⁴ to about 10⁵ cells/kg, about 10⁵ to about 10⁶ cells/kg, about 10⁶ to about 10⁷ cells/kg, about 10⁷ to about 10⁸ cells/kg, or about 10⁸ to about 10⁹ cells/kg; or at least about one of: 1×10⁷, 1.5×10⁷, 2×10⁷, 2.5×10⁷, 3×10⁷, 3.5×10⁷, 4×10⁷, 5×10⁷, 1×10⁸, 1.5×10⁸, 2×10⁸, 2.5×10⁸, 3×10⁸, 3.5×10⁸, 4×10⁸, 5×10⁸, 1×10⁹, 2×10⁹, or 5×10⁹ cells. In some embodiments, a dose of CAR-expressing cells (e.g., CD19 CAR-expressing cells) comprises at least about 1-5×10⁷ to 1-5×10⁸ CAR-expressing cells In some embodiments, the subject is administered about 1-5×10⁷ CAR-expressing cells (e.g., CD19 CAR-expressing cells). In other embodiments, the subject is administered about 1-5×10⁸ CAR-expressing cells (e.g., CD19 CAR-expressing cells).

In embodiments, the CAR-expressing cells (e.g., CD19 CAR-expressing cells) are administered to the subject according to a dosing regimen comprising a total dose of cells administered to the subject by dose fractionation, e.g., one, two, three or more separate administration of a partial dose. In embodiments, a first percentage of the total dose is administered on a first day of treatment, a second percentage of the total dose is administered on a subsequent (e.g., second, third, fourth, fifth, sixth, or seventh or later) day of treatment, and optionally, a third percentage (e.g., the remaining percentage) of the total dose is administered on a yet subsequent (e.g., third, fourth, fifth, sixth, seventh, eighth, ninth, tenth, or later) day of treatment. For example, 10% of the total dose of cells is delivered on the first day, 30% of the total dose of cells is delivered on the second day, and the remaining 60% of the total dose of cells is delivered on the third day of treatment. For example, a total cell dose includes 1 to 5×10⁷ or 1 to 5×10⁸ CAR-expressing cells (e.g., CD19 CAR-expressing cells).

In any of the administration regimens described herein, a dose of a PD-1 inhibitor, e.g., an anti-PD-1 antibody molecule described herein (e.g., pembrolizumab, nivolumab, PDR001, or an anti-PD-1 antibody molecule provided in Table 6), comprises about 1 to 30 mg/kg, e.g., about 1 to 20 mg/kg, about 2 to 15 mg/kg, about 5 to 25 mg/kg, about 10 to 20 mg/kg, about 1 to 5 mg/kg, about 2 mg/kg, about 3 mg/kg, or about 10 mg/kg. In one embodiment, the dose is about 10 to 20 mg/kg. In one embodiment, the dose is about 1 to 5 mg/kg. In one embodiment, the dose is less than 5 mg/kg, less than 4 mg/kg, less than 3 mg/kg, less than 2 mg/kg, or less than 1 mg/kg. In one embodiment, the dose is about 2 mg/kg.

In embodiments, in any of the administration regimens described herein, the dose of the PD-1 inhibitor is administered every 1-4 weeks, e.g., every week, every 2 weeks, every 3 weeks, or every 4 weeks.

In certain embodiments, the anti-PD-1 antibody molecule (e.g., pembrolizumab, nivolumab, PDR001, or an anti-PD-1 antibody molecule provided in Table 6) is administered by injection (e.g., subcutaneously or intravenously) at a dose of about 1 to 30 mg/kg, e.g., about 1 to 20 mg/kg, about 2 to 15 mg/kg, about 5 to 25 mg/kg, about 10 to 20 mg/kg, about 1 to 5 mg/kg, about 3 mg/kg, or about 2 mg/kg. The dosing schedule can vary from e.g., once a week to once every 2, 3, or 4 weeks. In one embodiment, the anti-PD-1 antibody molecule is administered at a dose from about 10 to 20 mg/kg every other week. In one embodiment, the dose is about 1 to 5 mg/kg every 2 weeks, every 3 weeks, or every 4 weeks. In one embodiment, the dose is less than 5 mg/kg, less than 4 mg/kg, less than 3 mg/kg, less than 2 mg/kg, or less than 1 mg/kg, every 2 weeks, every 3 weeks, or every 4 weeks. In one embodiment, the dose is about 2 mg/kg, every 2 weeks, every 3 weeks, or every 4 weeks.

In some embodiments, the dose of a PD-1 inhibitor, e.g., an anti-PD-1 antibody molecule (e.g., pembrolizumab, nivolumab, PDR001 or an anti-PD-1 antibody molecule provided in Table 6), is a flat dose. In some embodiments, the anti-PD-1 antibody molecule is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 200 mg, about 300 mg or about 400 mg. The dosing schedule (e.g., flat dosing schedule) can vary from, e.g., once a week to once every 2, 3, 4, 5, or 6 weeks. In one embodiment, the anti-PD-1 antibody molecule is administered at a dose from about 200 mg once every three weeks or once every four weeks. In one embodiment, the anti-PD-1 antibody molecule is administered at a dose from about 300 mg to 400 mg once every three weeks or once every four weeks. In one embodiment, the anti-PD-1 antibody molecule is administered at a dose from about 300 mg once every three weeks, e.g., via i.v. infusion. In one embodiment, the anti-PD-1 antibody molecule is administered at a dose from about 200 mg once every three weeks, e.g., via i.v. infusion. In one embodiment, the anti-PD-1 antibody molecule is administered at a dose from about 400 mg once every four weeks, e.g., via i.v. infusion. In one embodiment, the anti-PD-1 antibody molecule is administered at a dose from about 300 mg once every four weeks, e.g., via i.v. infusion. In one embodiment, the anti-PD-1 antibody molecule is administered at a dose from about 400 mg once every three weeks, e.g., via i.v. infusion.

In one embodiment, the PD-1 inhibitor is pembrolizumab administered at 200 mg every three weeks for up to six doses. In some embodiments, the PD-1 inhibitor is pembrolizumab administered at 300 mg every three weeks for up to six doses.

In one embodiment, the PD-1 inhibitor is selected from the group consisting of Nivolumab, Pembrolizumab, Pidilizumab, PDR001, AMP 514, AMP-224, and any anti-PD-1 antibody molecule provided in Table 6.

In some embodiments, the disclosure provides a method of treating a subject having a disease associated with expression of CD19, e.g., a hematologic cancer (e.g., DLBCL (e.g. primary DLBCL) or B-cell acute lymphoblastic leukemia (B-ALL)). The method comprises administering to the subject an effective number of a population of cells that express a CAR molecule that binds CD19, e.g., a CD19 CAR (“CD19 CAR therapy”) as described herein, in combination with a PD1 inhibitor, e.g., an anti-PD1 antibody as described herein. In some embodiments, the CD19 CAR therapy is administered prior to, simultaneously with or after the PD-1 inhibitor. In one embodiment, the CD19 CAR therapy is administered prior to the PD-1 inhibitor. For example, one or more doses of the PD-1 inhibitor can be administered post-CD19 CAR therapy (e.g., starting 5 days to 4 months, e.g., 10 day to 3 months, e.g., 14 days to 2 months post-CD19 CAR therapy). In some embodiments, the combination of the CD19 CAR therapy and PD-1 inhibitor therapy is repeated.

In one embodiment of the therapy comprising the CD19 CAR-expressing cell and the PD1 inhibitor, the CD19 CAR therapy comprises one or more treatments with cells that express a CD19 CAR as described herein. In embodiments, the CD19 CAR molecule comprises an antigen binding domain that binds specifically to CD19, e.g., as described herein. In embodiments, the CD19 CAR and PD-1 inhibitor therapies are administered at a dosage described herein.

In some embodiments, the CD19 CAR (or a nucleic acid encoding it) comprises a sequence set out in any of Table 2 or Table 3.

In embodiments of the therapy comprising the CD19 CAR-expressing cell and the PD1 inhibitor, the CD19 CAR therapy comprises one or more treatments with cells that express a murine CAR molecule described herein, e.g., a murine CD19 CAR molecule of Table 3 or having CDRs as set out in Tables 4 and 5. In embodiments, the CD19 CAR is CTL019, e.g., as described herein.

In another embodiment of the therapy comprising the CD19 CAR-expressing cell and the PD1 inhibitor, the CD19 CAR therapy comprises one or more treatments with cells that express a humanized CD19 CAR, e.g., a humanized CD19 CAR according to Table 2 or having CDRs as set out in Tables 4 and 5, e.g., CAR2 according to Table 2, e.g., CTL119.

In some embodiments, the CAR molecule comprises one, two, and/or three CDRs from the heavy chain variable region and/or one, two, and/or three CDRs from the light chain variable region of the murine or humanized CD19 CAR of Table 4 and 5.

In another embodiment of the therapy comprising the CD19 CAR-expressing cell and the PD1 inhibitor, the PD-1 inhibitor is an antibody to PD-1. In some embodiments, the PD-1 inhibitor is chosen from pembrolizumab, nivolumab, PDR001 (e.g., an antibody molecule of Table 6), MEDI-0680 (AMP-514), AMP-224, REGN-2810, or BGB-A317.

In one embodiment of the therapy comprising the CD19 CAR-expressing cell and the PD1 inhibitor, the PD-1 inhibitor is pembrolizumab. In one embodiment, the antibody molecule includes:
(i) a heavy chain variable (VH) region comprising the VHCDR1 amino acid sequence of SEQ ID NO: 503; the VHCDR2 amino acid sequence of SEQ ID NO: 504; and the VHCDR3 amino acid sequence of SEQ ID NO: 505; and
(ii) a light chain variable (VL) region comprising the VLCDR1 amino acid sequence of SEQ ID NO: 500; the VLCDR2 amino acid sequence of SEQ ID NO: 501; and rge VLCDR3 amino acid sequence of SEQ ID NO: 502,
   or an amino acid sequence at least 85%, 90%, 95% identical or higher.

In another embodiment of the therapy comprising the CD19 CAR-expressing cell and the PD1 inhibitor, the PD-1 inhibitor, e.g., the anti-PD-1 antibody molecule, includes at least one, two, three, four, five or six CDRs (or collectively all of the CDRs) from a heavy and light chain variable region from an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or in Table 6 herein; or encoded by the nucleotide sequence in Table 1, or encoded by the nucleotide sequence in Table 6 herein, or a sequence substantially identical (e.g., at least 80%, 85%, 90%, 92%, 95%, 97%, 98%, 99% or higher identical) to any of the aforesaid sequences; or closely related CDRs, e.g., CDRs which are identical or which have at least one amino acid alteration, but not more than two, three or four alterations (e.g., substitutions, deletions, or insertions, e.g., conservative substitutions).

In yet another embodiment of the therapy comprising the CD19 CAR-expressing cell and the PD1 inhibitor, the PD-1 inhibitor, e.g., the anti-PD-1 antibody molecule, comprises at least one, two, three or four variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or in Table 6 herein; or encoded by the nucleotide sequence in Table 1; or encoded by the nucleotide sequence in Table 6 herein, or a sequence substantially identical (e.g., at least 80%, 85%, 90%, 92%, 95%, 97%, 98%, 99% or higher identical) to any of the aforesaid sequences.

In embodiments, the PD-1 inhibitor, e.g., anti-PD-1 antibody molecule, is PDR-001, which contains the variable light chain and variable heavy chain amino acid sequences of BAP049-Clone-E, as described in Table 6.

In one embodiment of the therapy comprising the CD19 CAR-expressing cell and the PD1 inhibitor, the PD-1 inhibitor, e.g., pembrolizumab, is administered post-CD19 CAR therapy (e.g., starting 5 days to 4 months, e.g., 10 day to 3 months, e.g., 14 days to 2 months post-CTL019 or post-CTL119 therapy, or post- a combination of CTL019 and CTL119 therapies). In embodiments, administration of the therapy is to a subject with B-ALL, e.g., relapsed or refractory B-ALL.

In yet another embodiment of the therapy comprising the CD19 CAR-expressing cell and the PD1 inhibitor, the hematologic cancer is B-ALL or DLBCL, e.g., relapsed or refractory B-ALL or DLBCL. In one embodiment, the subject has a hematologic malignancy, e.g., B-ALL or DLBCL, and may not respond to the CAR T therapy or may relapse, e.g., due to poor CAR T cell persistence. In one embodiment of the CD19 CAR therapy-PD1 inhibitor therapy, the subject shows an improved therapeutic outcome, e.g., the subject achieves one or more of partial remission, complete remission, or prolonged CAR T cell persistence, in response to the CD19 CAR therapy-PD1 inhibitor therapy, e.g., one or more cycles of the CD19 CAR therapy-PD1 inhibitor therapy.

In one embodiment of the therapy comprising the CD19 CAR-expressing cell and the PD1 inhibitor, prior to administration of the PD-1 inhibitor, the subject has relapsed or refractory B-ALL or DLBCL to a prior treatment with a CD19 CAR therapy, e.g., a prior treatment with one or both of CTL019 and CTL119. In some embodiments, the subject shows decreased or poor CAR T cell persistence. In some embodiments, the subject is, or has been treated with CTL019 followed by CTL119.

In some embodiments, the subject shows CD19+ relapse. In some embodiments, the subject has relapsed or refractory CD19+ B-ALL. In some embodiments, the subject has relapsed or refractory CD19+ DLBCL. In one embodiment, the subject has relapsed or refractory B-ALL with lymph node involvement, e.g., has lymphomatous disease.

In some embodiments, the subject that has relapsed or refractory B-ALL with lymph node involvement, e.g., has lymphomatous disease, to a prior treatment with a CD19 CAR therapy, shows decreased PET-avid lesions, e.g., shows a reduced number of or intensity of lesions, in response to the CD19 CAR therapy-PD1 inhibitor therapy, e.g., in response to one or more cycles of the CD19 CAR therapy-PD1 inhibitor therapy.

In some embodiments, the subject, e.g., a subject showing CD19+ relapse after a CD19CAR therapy, is administered a further CD19 CAR therapy, in combination with the PD-1 inhibitor, e.g., pembrolizumab. In embodiments, the further administration of the combination therapy results in an improved therapeutic outcome, e.g., the subject achieves one or more of partial remission, complete remission, or a prolonged CAR T cell persistence. In an embodiment, the administration of the combination therapy results in prolonged persistence of a CAR T cell, e.g., a CD19 CAR-expressing cell. In an embodiment, the administration of the combination therapy results in a longer time for B cell recovery, e.g., longer time prior to B cell aplasia, e.g., compared to a subject treated with CD19 CAR therapy alone. In some embodiments, the subject after treatment with the combination disclosed herein has one or more of: (i) a decreased risk of relapse, (ii) delayed timing of the onset of relapse, or (iii) decreased severity of relapse, e.g., compared to a subject treated with CD19 CAR therapy alone. In an embodiment, administration of the combination therapy results in an objective clinical response.

In an embodiment, the subject, e.g., a subject showing relapse after a CD19 CAR therapy, is eligible to receive repeat administration of a CD19 CAR therapy, e.g., a second, third or fourth dose. In an embodiment, the subject is eligible to receive a repeat administration of a CD19 CAR therapy, e.g., a second, third or fourth dose, along with a PD-1 inhibitor. In an embodiment, a subject showing low persistence of CD19 CAR therapy after a first administration of a CD19 CAR therapy is eligible to receive a repeat administration of a CD19 CAR therapy, e.g., a second, third or fourth dose, along with a PD-1 inhibitor.

Optionally, the subject has, or is identified as having, at least 5%, 6%, 7%, 8%, 9%, 10%, 20%, 30%, 40%, 50%, 60%, 70%, 80%, or 90% of cancer cells, e.g., DLBCL cells, which are CD3+/PD1+.

In another aspect, the disclosure features a composition (e.g., one or more dosage formulations, combinations, or one or more pharmaceutical compositions) comprising a cell expressing a CAR (e.g., CD19 CAR) described herein and a PD-1 inhibitor described herein. In one embodiment, the CAR (e.g., CD19 CAR) comprises an antigen binding domain (e.g., CD19 antigen binding domain), a transmembrane domain, and an intracellular signaling domain, as described herein. In one embodiment, the CD19 CAR comprises a CD19 antigen binding domain listed in Table 2 or 3. In one embodiment, the PD-1 inhibitor comprises an antibody molecule, a small molecule, a polypeptide, e.g., a fusion protein, or an inhibitory nucleic acid, e.g., a siRNA or shRNA. In one embodiment, the PD-1 inhibitor comprises an antibody molecule, e.g., pembrolizumab, nivolumab, PDR001 or an antibody molecule listed in Table 6. The CAR-expressing cell and the PD-1 inhibitor can be in the same or different formulation or pharmaceutical composition.

In another aspect, the disclosure features a composition (e.g., one or more dosage formulations, combinations, or one or more pharmaceutical compositions) comprising a cell expressing a CAR (e.g., CD19 CAR) described herein and a PD-1 inhibitor described herein, for use in a method of treating a disease (e.g., cancer), e.g., disease associated with expression of CD19, e.g., a cancer described herein. In one embodiment, the CAR (e.g., CD19 CAR) comprises an antigen binding domain (e.g., CD19 antigen binding domain), a transmembrane domain, and an intracellular signaling domain, as described herein. In one embodiment, the CD19 CAR comprises a CD19 antigen binding domain listed in Table 2 or 3. In one embodiment, the PD-1 inhibitor comprises an antibody molecule, a small molecule, a polypeptide, e.g., a fusion protein, or an inhibitory nucleic acid, e.g., a siRNA or shRNA. In one embodiment, the PD-1 inhibitor comprises an antibody molecule, e.g., pembrolizumab, nivolumab, PDR001, or an antibody molecule listed in Table 6. The CAR-expressing cell and the PD-1 inhibitor can be in the same or different formulation or pharmaceutical composition.

### PD-1 Inhibitors

Provided herein are PD-1 inhibitors for use in any of the methods or compositions described herein. In any of the methods or compositions described herein, the PD-1 inhibitor comprises an antibody molecule, a small molecule, a polypeptide, e.g., a fusion protein, or an inhibitory nucleic acid, e.g., a siRNA or shRNA.

In one embodiment, the PD-1 inhibitor is characterized by one or more of the following: inhibits or reduces PD-1 expression, e.g., transcription or translation of PD-1; inhibits or reduces PD-1 activity, e.g., inhibits or reduces binding of PD-1 to its ligand, e.g., PD-L1; or binds to PD-1 or its ligand, e.g., PD-L1.

In one embodiment, the PD-1 inhibitor is an antibody molecule.

In one embodiment, the PD-1 inhibitor comprises an anti-PD-1 antibody molecule comprising a heavy chain complementary determining region 1 (HC CDR1), a heavy chain complementary determining region 2 (HC CDR2), and a heavy chain complementary determining region 3 (HC CDR3) of any PD-1 antibody molecule amino acid sequence listed in Table 6; and/or a light chain complementary determining region 1 (LC CDR1), a light chain complementary determining region 2 (LC CDR2), and a light chain complementary determining region 3 (LC CDR3) of any PD-1 antibody molecule amino acid sequence listed in Table 6. In one embodiment, the anti-PD1 antibody molecule comprises a HC CDR1 amino acid sequence chosen from SEQ ID NO: 137 or 140, a HC CDR2 amino acid sequence of SEQ ID NO: 138 or 141, and a HC CDR3 amino acid sequence of SEQ ID NO: 139; and/or a LC CDR1 amino acid sequence of SEQ ID NO: 146 or 149, a LC CDR2 amino acid sequence of SEQ ID NO: 147 or 150, and a LC CDR3 amino acid sequence of SEQ ID NO: 148, 151, 166, or 167. In one embodiment, the anti-PD-1 antibody comprises a HC CDR1 amino acid sequence chosen from SEQ ID NO: 137 or 140, a HC CDR2 amino acid sequence of SEQ ID NO: 138 or 141, and a HC CDR3 amino acid sequence of SEQ ID NO: 139; and/or a LC CDR1 amino acid sequence of SEQ ID NO: 146 or 149, a LC CDR2 amino acid sequence of SEQ ID NO: 147 or 150, and a LC CDR3 amino acid sequence of SEQ ID NO: 166 or 167.

In one embodiment, the anti-PD-1 antibody molecule comprises a heavy chain variable region comprising the amino acid sequence of any heavy chain variable region listed in Table 6, e.g., SEQ ID NOs: 142, 144, 154, 158, 154, 158, 172, 184, 216, or 220. In one embodiment, the anti-PD-1 antibody molecule comprises a heavy chain variable region comprising the amino acid sequence having at least one, two or three modifications but not more than 30, 20 or 10 modifications to the amino acid sequence of any heavy chain variable region provided in Table 6, e.g., SEQ ID NOs: 142, 144, 154, 158, 154, 158, 172, 184, 216, or 220. In one embodiment, the anti-PD-1 antibody molecule comprises a heavy chain variable region comprising an amino acid sequence at least 95% identical (e.g., with 95-99% identity) to the amino acid sequence of any heavy chain variable region provided in Table 6, e.g., SEQ ID NOs: 142, 144, 154, 158, 154, 158, 172, 184, 216, or 220.

In one embodiment, the anti-PD-1 antibody molecule comprises a heavy chain comprising the amino acid sequence of any heavy chain listed in Table 6, e.g., SEQ ID NOs:156, 160, 174, 186, 218, 222, 225, or 236. In one embodiment, the anti-PD-1 antibody molecule comprises a heavy chain comprising the amino acid sequence having at least one, two or three modifications but not more than 30, 20 or 10 modifications to any heavy chain listed in Table 6, e.g., SEQ ID NOs: 156, 160, 174, 186, 218, 222, 225, or 236. In one embodiment, the anti-PD-1 antibody molecule comprises a heavy chain comprising an amino acid sequence with 95-99% identity to the amino acid sequence of any heavy chain listed in Table 6, e.g., SEQ ID NOs: 156, 160, 174, 186, 218, 222, 225, or 236.

In one embodiment, the anti-PD-1 antibody molecule comprises a light chain variable region comprising the amino acid sequence of any light chain variable region listed in Table 6, e.g., SEQ ID NOs: 152, 162, 168, 176, 180, 188, 192, 196, 200, 204, 208, or 212. In one embodiment, the anti-PD-1 antibody molecule comprises a light chain variable region comprising the amino acid sequence having at least one, two or three modifications but not more than 30, 20 or 10 modifications to the amino acid sequence of any light chain variable region provided in Table 6, e.g., SEQ ID NOs: 152, 162, 168, 176, 180, 188, 192, 196, 200, 204, 208, or 212. In one embodiment, the anti-PD-1 antibody molecule comprises a light chain variable region comprising an amino acid sequence at least 95% identical (e.g., with 95-99% identity) to the amino acid sequence of any light chain variable region provided in Table 6, e.g., SEQ ID NOs: 152, 162, 168, 176, 180, 188, 192, 196, 200, 204, 208, or 212.

In one embodiment, the anti-PD-1 antibody molecule comprises a light chain comprising the amino acid sequence of any light chain listed in Table 6, e.g., SEQ ID NOs: 164, 170, 178, 182, 190, 194, 198, 202, 206, 210, or 214. In one embodiment, the anti-PD-1 antibody molecule comprises a light chain comprising the amino acid sequence having at least one, two or three modifications but not more than 30, 20 or 10 modifications to any light chain listed in Table 6, e.g., SEQ ID NOs: 164, 170, 178, 182, 190, 194, 198, 202, 206, 210, or 214. In one embodiment, the anti-PD-1 antibody molecule comprises a light chain comprising an amino acid sequence at least 95% identical (e.g., with 95-99% identity) to the amino acid sequence to any any light chain listed in Table 6, e.g., SEQ ID NOs: 164, 170, 178, 182, 190, 194, 198, 202, 206, 210, or 214.

In one embodiment, the anti-PD-1 antibody molecule comprises:
i) a heavy chain comprising the amino acid sequence of SEQ ID NO: 144 and a light chain comprising the amino acid sequence of SEQ ID NO: 152;
ii) a heavy chain comprising the amino acid sequence of SEQ ID NO: 156 or 160 and a light chain comprising the amino acid sequence of SEQ ID NO: 164;
iii) a heavy chain comprising the amino acid sequence of SEQ ID NO: 156 or 160 and a light chain comprising the amino acid sequence of SEQ ID NO: 170.
iv) a heavy chain comprising the amino acid sequence of SEQ ID NO: 174 and a light chain comprising the amino acid sequence of SEQ ID NO: 178;
v) a heavy chain comprising the amino acid sequence of SEQ ID NO: 174 and a light chain comprising the amino acid sequence of SEQ ID NO: 182;
vi) a heavy chain comprising the amino acid sequence of SEQ ID NO: 186 and a light chain comprising the amino acid sequence of SEQ ID NO: 182;
vii) a heavy chain comprising the amino acid sequence of SEQ ID NO: 186 and a light chain comprising the amino acid sequence of SEQ ID NO: 190;
viii) a heavy chain comprising the amino acid sequence of SEQ ID NO: 174 and a light chain comprising the amino acid sequence of SEQ ID NO: 190;
ix) a heavy chain comprising the amino acid sequence of SEQ ID NO: 174 and a light chain comprising the amino acid sequence of SEQ ID NO: 194;
x) a heavy chain comprising the amino acid sequence of SEQ ID NO: 174 and a light chain comprising the amino acid sequence of SEQ ID NO: 198;
xi) a heavy chain comprising the amino acid sequence of SEQ ID NO: 186 and a light chain comprising the amino acid sequence of SEQ ID NO: 202;
xii) a heavy chain comprising the amino acid sequence of SEQ ID NO: 174 and a light chain comprising the amino acid sequence of SEQ ID NO: 202;
xiii) a heavy chain comprising the amino acid sequence of SEQ ID NO: 186 and a light chain comprising the amino acid sequence of SEQ ID NO: 206;
xiv) a heavy chain comprising the amino acid sequence of SEQ ID NO: 174 and a light chain comprising the amino acid sequence of SEQ ID NO: 206;
xv) a heavy chain comprising the amino acid sequence of SEQ ID NO: 174 and a light chain comprising the amino acid sequence of SEQ ID NO: 210;
xvi) a heavy chain comprising the amino acid sequence of SEQ ID NO: 174 and a light chain comprising the amino acid sequence of SEQ ID NO: 214;
xvii) a heavy chain comprising the amino acid sequence of SEQ ID NO: 218 and a light chain comprising the amino acid sequence of SEQ ID NO: 206;
xviii) a heavy chain comprising the amino acid sequence of SEQ ID NO: 218 and a light chain comprising the amino acid sequence of SEQ ID NO: 202;
xix) a heavy chain comprising the amino acid sequence of SEQ ID NO: 222 and a light chain comprising the amino acid sequence of SEQ ID NO: 202;
xx) a heavy chain comprising the amino acid sequence of SEQ ID NO: 225 and a light chain comprising the amino acid sequence of SEQ ID NO: 178;
xxi) a heavy chain comprising the amino acid sequence of SEQ ID NO: 225 and a light chain comprising the amino acid sequence of SEQ ID NO: 190;
xxii) a heavy chain comprising the amino acid sequence of SEQ ID NO: 225 and a light chain comprising the amino acid sequence of SEQ ID NO: 202;
xxiii) a heavy chain comprising the amino acid sequence of SEQ ID NO: 236 and a light chain comprising the amino acid sequence of SEQ ID NO: 206; or
xxiv) a heavy chain comprising the amino acid sequence of SEQ ID NO: 225 and a light chain comprising the amino acid sequence of SEQ ID NO: 206.

In one embodiment, the anti-PD-1 antibody molecule comprises:
i) a heavy chain variable domain comprising the amino acid sequence of SEQ ID NO: 172 and a light chain variable domain comprising the amino acid sequence of SEQ ID NO: 204;
ii) a heavy chain variable domain comprising the amino acid sequence of SEQ ID NO: 142 or 144 and a light chain variable domain comprising the amino acid sequence of SEQ ID NO: 152;
iii) a heavy chain variable domain comprising the amino acid sequence of SEQ ID NO: 154 or 158 and a light chain variable domain comprising the amino acid sequence of SEQ ID NO: 162;
iv) a heavy chain variable domain comprising the amino acid sequence of SEQ ID NO: 154 or 158 and a light chain variable domain comprising the amino acid sequence of SEQ ID NO: 168;
v) a heavy chain variable domain comprising the amino acid sequence of SEQ ID NO: 172 and a light chain variable domain comprising the amino acid sequence of SEQ ID NO: 176;
vi) a heavy chain variable domain comprising the amino acid sequence of SEQ ID NO: 172 and a light chain variable domain comprising the amino acid sequence of SEQ ID NO: 180;
vii) a heavy chain variable domain comprising the amino acid sequence of SEQ ID NO: 184 and a light chain variable domain comprising the amino acid sequence of SEQ ID NO: 180;
viii) a heavy chain variable domain comprising the amino acid sequence of SEQ ID NO: 184 and a light chain variable domain comprising the amino acid sequence of SEQ ID NO: 188;
ix) a heavy chain variable domain comprising the amino acid sequence of SEQ ID NO: 172 and a light chain variable domain comprising the amino acid sequence of SEQ ID NO: 188;
x) a heavy chain variable domain comprising the amino acid sequence of SEQ ID NO: 172 and a light chain variable domain comprising the amino acid sequence of SEQ ID NO: 192;
xi) a heavy chain variable domain comprising the amino acid sequence of SEQ ID NO: 172 and a light chain variable domain comprising the amino acid sequence of SEQ ID NO: 196;
xii) a heavy chain variable domain comprising the amino acid sequence of SEQ ID NO: 184 and a light chain variable domain comprising the amino acid sequence of SEQ ID NO: 200;
xiii) a heavy chain variable domain comprising the amino acid sequence of SEQ ID NO: 172 and a light chain variable domain comprising the amino acid sequence of SEQ ID NO: 200;
xiv) a heavy chain variable domain comprising the amino acid sequence of SEQ ID NO: 184 and a light chain variable domain comprising the amino acid sequence of SEQ ID NO: 204;
xv) a heavy chain variable domain comprising the amino acid sequence of SEQ ID NO: 172 and a light chain variable domain comprising the amino acid sequence of SEQ ID NO: 204;
xvi) a heavy chain variable domain comprising the amino acid sequence of SEQ ID NO: 172 and a light chain variable domain comprising the amino acid sequence of SEQ ID NO: 208;
xvii) a heavy chain variable domain comprising the amino acid sequence of SEQ ID NO: 172 and a light chain variable domain comprising the amino acid sequence of SEQ ID NO: 212;
xviii) a heavy chain variable domain comprising the amino acid sequence of SEQ ID NO: 216 and a light chain variable domain comprising the amino acid sequence of SEQ ID NO: 204;
xix) a heavy chain variable domain comprising the amino acid sequence of SEQ ID NO: 216 and a light chain variable domain comprising the amino acid sequence of SEQ ID NO: 200;
xx) a heavy chain variable domain comprising the amino acid sequence of SEQ ID NO: 220 and a light chain variable domain comprising the amino acid sequence of SEQ ID NO: 200;
xxi) a heavy chain variable domain comprising the amino acid sequence of SEQ ID NO: 172 and a light chain variable domain comprising the amino acid sequence of SEQ ID NO: 176;
xxii) a heavy chain variable domain comprising the amino acid sequence of SEQ ID NO: 172 and a light chain variable domain comprising the amino acid sequence of SEQ ID NO: 188;
xxiii) a heavy chain variable domain comprising the amino acid sequence of SEQ ID NO: 172 and a light chain variable domain comprising the amino acid sequence of SEQ ID NO: 200; or
xxiv) a heavy chain variable domain comprising the amino acid sequence of SEQ ID NO: 184 and a light chain variable domain comprising the amino acid sequence of SEQ ID NO: 204.

In one embodiment, the anti-PD-1 antibody molecule includes at least one or two heavy chain variable domain (optionally including a constant region), at least one or two light chain variable domain (optionally including a constant region), or both, comprising the amino acid sequence of BAP049-Clone-A, BAP049-Clone-B, BAP049-Clone-C, BAP049-Clone-D, or BAP049-Clone-E; or as described in Table 1 of US 2015/0210769, or in Table 6 herein, or encoded by the nucleotide sequence in Table 6; or a sequence substantially identical (e.g., at least 80%, 85%, 90%, 92%, 95%, 97%, 98%, 99% or higher identical) to any of the aforesaid sequences. The anti-PD-1 antibody molecule, optionally, comprises a leader sequence from a heavy chain, a light chain, or both, as shown in Table 4 of US 2015/0210769; or a sequence substantially identical thereto.

In yet another embodiment, the anti-PD-1 antibody molecule includes at least one, two, or three complementarity determining regions (CDRs) from a heavy chain variable region and/or a light chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule includes at least one, two, or three CDRs (or collectively all of the CDRs) from a heavy chain variable region comprising an amino acid sequence shown in Table 1 of US 2015/0210769, or in Table 6 herein, or encoded by a nucleotide sequence shown in Table 6. In one embodiment, one or more of the CDRs (or collectively all of the CDRs) have one, two, three, four, five, six or more changes, e.g., amino acid substitutions or deletions, relative to the amino acid sequence shown in Table 6, or encoded by a nucleotide sequence shown in Table 6.

In yet another embodiment, the anti-PD-1 antibody molecule includes at least one, two, or three CDRs (or collectively all of the CDRs) from a light chain variable region comprising an amino acid sequence shown in Table 1 of US 2015/0210769, or in Table 6 herein, or encoded by a nucleotide sequence shown in Table 6. In one embodiment, one or more of the CDRs (or collectively all of the CDRs) have one, two, three, four, five, six or more changes, e.g., amino acid substitutions or deletions, relative to the amino acid sequence shown in Table 6, or encoded by a nucleotide sequence shown in Table 6. In certain embodiments, the anti-PD-1 antibody molecule includes a substitution in a light chain CDR, e.g., one or more substitutions in a CDR1, CDR2 and/or CDR3 of the light chain. In one embodiment, the anti-PD-1 antibody molecule includes a substitution in the light chain CDR3 at position 102 of the light variable region, e.g., a substitution of a cysteine to tyrosine, or a cysteine to serine residue, at position 102 of the light variable region according to Table 6 (e.g., SEQ ID NO: 152 or 162 for murine or chimeric, unmodified; or any of SEQ ID NOs: 168, 176, 180, 188, 192, 196, 200, 204, 208, or 212 for a modified sequence).

In another embodiment, the anti-PD-1 antibody molecule includes at least one, two, three, four, five or six CDRs (or collectively all of the CDRs) from a heavy and light chain variable region comprising an amino acid sequence shown in Table 1 of US 2015/0210769, or in Table 6 herein, or encoded by a nucleotide sequence shown in Table 6. In one embodiment, one or more of the CDRs (or collectively all of the CDRs) have one, two, three, four, five, six or more changes, e.g., amino acid substitutions or deletions, relative to the amino acid sequence shown in Table 6, or encoded by a nucleotide sequence shown in Table 6.

In one embodiment, the anti-PD-1 antibody molecule includes:

(a) a heavy chain variable region (VH) comprising a VHCDR1 amino acid sequence of SEQ ID NO: 140, a VHCDR2 amino acid sequence of SEQ ID NO: 141, and a VHCDR3 amino acid sequence of SEQ ID NO: 139; and a light chain variable region (VL) comprising a VLCDR1 amino acid sequence of SEQ ID NO: 149, a VLCDR2 amino acid sequence of SEQ ID NO: 150, and a VLCDR3 amino acid sequence of SEQ ID NO: 167, each disclosed in Table 1 of US 2015/0210769, or in Table 6 herein;

(b) a VH comprising a VHCDR1 amino acid sequence chosen from SEQ ID NO: 137; a

VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166, each disclosed in Table 1 of US 2015/0210769, or in Table 6 herein;

(c) a VH comprising a VHCDR1 amino acid sequence of SEQ ID NO: 286, a VHCDR2 amino acid sequence of SEQ ID NO: 141, and a VHCDR3 amino acid sequence of SEQ ID NO: 139; and a VL comprising a VLCDR1 amino acid sequence of SEQ ID NO: 149, a VLCDR2 amino acid sequence of SEQ ID NO: 150, and a VLCDR3 amino acid sequence of SEQ ID NO: 167, each disclosed in Table 1 of US 2015/0210769, or in Table 6 herein; or

(d) a VH comprising a VHCDR1 amino acid sequence of SEQ ID NO: 286; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166, each disclosed in Table 1 of US 2015/0210769, or in Table 6 herein.

In the combinations herein below, in another embodiment, the anti-PD-1 antibody molecule comprises (i) a heavy chain variable region (VH) comprising a VHCDR1 amino acid sequence chosen from SEQ ID NO: 137, SEQ ID NO: 140, or SEQ ID NO: 286; a VHCDR2 amino acid sequence of SEQ ID NO: 138 or SEQ ID NO: 141; and a VHCDR3 amino acid sequence of SEQ ID NO: 139; and (ii) a light chain variable region (VL) comprising a VLCDR1 amino acid sequence of SEQ ID NO: 146 or SEQ ID NO: 149, a VLCDR2 amino acid sequence of SEQ ID NO: 147 or SEQ ID NO: 150, and a VLCDR3 amino acid sequence of SEQ ID NO: 166 or SEQ ID NO: 167, each disclosed in Table 1 of US 2015/0210769, or in Table 6 herein.

In embodiments, the PD-1 inhibitor, e.g., anti-PD-1 antibody molecule, is PDR-001, which contains the variable light chain and variable heavy chain amino acid sequences of BAP049-Clone-E, as described in Table 6. In one embodiment, the anti-PD-1 antibody molecule comprises a heavy chain variable domain comprising the amino acid sequence of SEQ ID NO: 172 and a light chain variable domain comprising the amino acid sequence of SEQ ID NO: 204.

In some embodiments, the PD-1 inhibitor is chosen from Nivolumab, Pembrolizumab, Pidilizumab, AMP 514, AMP-224, or an anti-PD1 antibody described in U.S. Pat. No. 8,609,089, US 2010028330, and/or US 20120114649, each of which is incorporated herein by reference in its entirety.

In one embodiment, the PD-1 inhibitor is pembrolizumab. In one embodiment, the antibody molecule includes:
(i) a heavy chain variable (VH) region comprising the VHCDR1 amino acid sequence of SEQ ID NO: 503; the VHCDR2 amino acid sequence of SEQ ID NO: 504; and the VHCDR3 amino acid sequence of SEQ ID NO: 505; and
(ii) a light chain variable (VL) region comprising the VLCDR1 amino acid sequence of SEQ ID NO: 500; the VLCDR2 amino acid sequence of SEQ ID NO: 501; and rge VLCDR3 amino acid sequence of SEQ ID NO: 502,
   or an amino acid sequence at least 85%, 90%, 95% identical or higher.

### CAR-Expressing Cells

Provided herein are cells, e.g., immune effector cells, that express a chimeric antigen receptor (CAR) that targets, e.g., specifically binds to, an antigen (e.g., CD19), for use in any of the methods or compositions described herein. The CAR that specifically binds to antigen X is also referred to herein as an “X CAR”. For example, the CAR that specifically binds to CD19 also referred to herein as “a CD19 CAR”. The CAR (e.g., CD19 CAR) expressed by the CAR-expressing cell (e.g., CD19 CAR-expressing cell) described herein includes an antigen binding domain (e.g., CD19 binding domain), a transmembrane domain, and an intracellular signaling domain. In one embodiment, the intracellular signaling domain comprises a costimulatory domain and/or a primary signaling domain.

In embodiments, the CAR molecule comprises an antigen binding domain, transmembrane domain, and an intracellular signaling domain (e.g., an intracellular signaling domain comprising a costimulatory domain and/or a primary signaling domain).

In one embodiment, the CAR molecule comprises an antigen binding domain that is capable of binding an antigen described herein, e.g., a tumor antigen, e.g., chosen from one or more of the following: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGaip(1-4)bDGicp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGicp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member lA (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRLS); and immunoglobulin lambda-like polypeptide 1 (IGLL1).

In one embodiment, the antigen binding domain of the CAR binds to a B cell antigen, e.g., CD10, CD19, CD20, CD22, CD34, CD123, FLT-3, ROR1, CD79b, CD179b, and/or CD79a.

In embodiments, the antigen binding domain of the CAR binds to CD123.

In embodiments, the antigen binding domain of the CAR binds to CD19.

In other embodiments, the antigen binding domain of the CAR binds to BCMA.

In embodiments, the antigen binding domain of the CAR binds to CLL.

### CD19 Antigen Binding Domain

In one embodiment, the CD19 binding domain comprises a heavy chain complementary determining region 1 (HC CDR1), a heavy chain complementary determining region 2 (HC CDR2), and a heavy chain complementary determining region 3 (HC CDR3) of any CD19 heavy chain binding domain amino acid sequence listed in Table 2 or 3; and a light chain complementary determining region 1 (LC CDR1), a light chain complementary determining region 2 (LC CDR2), and a light chain complementary determining region 3 (LC CDR3) of any CD19 light chain binding domain amino acid sequence listed in Table 2 or 3. In one embodiment, the CD19 binding domain comprises a HC CDR1, a HC CDR2, and a HC CDR3 according to the HC CDR amino acid sequences in Table 4, and a LC CDR1, a LC CDR2, and a LC CDR3 according to the LC CDR amino acid sequences in Table 5.

In one embodiment, the CD19 binding domain comprises (e.g., consists of) the amino acid sequence selected from the group consisting of SEQ ID NO: 109, SEQ ID NO: 45, SEQ ID NO: 46, SEQ ID NO: 47, SEQ ID NO:48, SEQ ID NO:49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 110, SEQ ID NO: 112, or SEQ ID NO: 115. In one embodiment, the CD19 binding domain comprises (e.g., consists of) an amino acid sequence having at least one, two or three modifications but not more than 30, 20 or 10 modifications (e.g., substitutions, e.g., conservative substitutions) to any of SEQ ID NO: 109, SEQ ID NO: 45, SEQ ID NO: 46, SEQ ID NO: 47, SEQ ID NO:48, SEQ ID NO:49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 110, SEQ ID NO: 112, or SEQ ID NO: 115. In one embodiment, the CD19 binding domain comprises (e.g., consists of) an amino acid sequence with 95-99% identity to the amino acid sequence to any of SEQ ID NO: 109, SEQ ID NO: 45, SEQ ID NO: 46, SEQ ID NO: 47, SEQ ID NO:48, SEQ ID NO:49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 110, SEQ ID NO: 112, or SEQ ID NO: 115.

### Additional Domains of a CAR Molecule

In one embodiment, the CAR, e.g., CD19 CAR, includes a transmembrane domain that comprises a transmembrane domain of a protein, e.g., a protein described herein, e.g., selected from the group consisting of the alpha, beta or zeta chain of the T-cell receptor, CD28, CD3 epsilon, CD45, CD4, CDS, CD8, CD9, CD16, CD22, CD33, CD37, CD64, CD80, CD86, CD134, CD137 and CD154. In one embodiment, the transmembrane domain comprises the sequence of SEQ ID NO: 6. In one embodiment, the transmembrane domain comprises an amino acid sequence comprising at least one, two or three modifications but not more than 20, 10 or 5 modifications of the amino acid sequence of SEQ ID NO:6, or a sequence with 95-99% identity to an amino acid sequence of SEQ ID NO:6. In one embodiment, the nucleic acid sequence encoding the transmembrane domain comprises a nucleotide sequence of SEQ ID NO: 17, or a sequence at least 95% identical (e.g., with 95-99% identity) thereof.

In one embodiment, the antigen binding domain (e.g., CD19 binding domain) is connected to the transmembrane domain by a hinge region, e.g., a hinge region described herein. In one embodiment, the encoded hinge region comprises SEQ ID NO: 2, or a sequence at least 95% identical (e.g., with 95-99% identity) thereof. In one embodiment, the nucleic acid sequence encoding the hinge region comprises a nucleotide sequence of SEQ ID NO: 13, or a sequence at least 95% identical (e.g., with 95-99% identity) thereof.

In one embodiment, the isolated nucleic acid molecule further comprises a sequence encoding a costimulatory domain, e.g., a costimulatory domain described herein. In embodiments, the intracellular signaling domain comprises a costimulatory domain. In embodiments, the intracellular signaling domain comprises a primary signaling domain. In embodiments, the intracellular signaling domain comprises a costimulatory domain and a primary signaling domain.

In one embodiment, the costimulatory domain is a functional signaling domain from a protein, e.g., described herein, e.g., selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In one embodiment, the costimulatory domain of 4-1BB comprises the amino acid sequence of SEQ ID NO: 7. In one embodiment, the encoded costimulatory domain comprises an amino acid sequence having at least one, two or three modifications but not more than 20, 10 or 5 modifications of an amino acid sequence of SEQ ID NO: 7, or a sequence at least 95% identical (e.g., with 95-99% identity) to the amino acid sequence of SEQ ID NO: 7. In one embodiment, the nucleic acid sequence encoding the costimulatory domain comprises the nucleotide sequence of SEQ ID NO: 18, or a sequence at least 95% identical (e.g., with 95-99% identity) thereof. In another embodiment, the costimulatory domain of CD28 comprises the amino acid sequence of SEQ ID NO: 36. In one embodiment, the costimulatory domain comprises an amino acid sequence having at least one, two or three modifications but not more than 20, 10 or 5 modifications of an amino acid sequence of SEQ ID NO: 36, or a sequence with 95-99% identity to an amino acid sequence of SEQ ID NO: 36. In one embodiment, the nucleic acid sequence encoding the costimulatory domain of CD28 comprises the nucleotide sequence of SEQ ID NO: 37, or a sequence at least 95% identical (e.g., with 95-99% identity) thereof. In another embodiment, the costimulatory domain of CD27 comprises the amino acid sequence of SEQ ID NO: 8. In one embodiment, the costimulatory domain comprises an amino acid sequence having at least one, two or three modifications but not more than 20, 10 or 5 modifications of an amino acid sequence of SEQ ID NO: 8, or a sequence at least 95% identical (e.g., with 95-99% identity) to an amino acid sequence of SEQ ID NO: 8. In one embodiment, the nucleic acid sequence encoding the costimulatory domain of CD27 comprises the nucleotide sequence of SEQ ID NO: 19, or a sequence at least 95% identical (e.g., with 95-99% identity) thereof. In another embodiment, the costimulatory domain of ICOS comprises the amino acid sequence of SEQ ID NO: 38. In one embodiment, the costimulatory domain of ICOS comprises an amino acid sequence having at least one, two or three modifications but not more than 20, 10 or 5 modifications of an amino acid sequence of SEQ ID NO: 38, or a sequence with 95-99% identity to an amino acid sequence of SEQ ID NO: 38. In one embodiment, the nucleic acid sequence encoding the costimulatory domain of ICOS comprises the nucleotide sequence of SEQ ID NO: 44, or a sequence at least 95% identical (e.g., with 95-99% identity) thereof. In embodiments, the costimulatory domain comprises an ICOS costimulatory domain mutant (e.g., Y to F mutant) comprising the amino acid sequence of SEQ ID NO: 43.

In some embodiments, the primary signaling domain comprises a functional signaling domain of CD3 zeta. In embodiments, the functional signaling domain of CD3 zeta comprises the amino acid sequence of SEQ ID NO: 9 (mutant CD3 zeta) or SEQ ID NO: 10 (wild type human CD3 zeta), or a sequence at least 95% identical (e.g., with 95-99% identity) thereof.

In one embodiment, the intracellular signaling domain comprises a functional signaling domain of 4-1BB and/or a functional signaling domain of CD3 zeta. In one embodiment, the intracellular signaling domain of 4-1BB comprises the sequence of SEQ ID NO: 7 and/or the CD3 zeta amino acid sequence of SEQ ID NO: 9 or SEQ ID NO: 10. In one embodiment, the intracellular signaling domain comprises an amino acid sequence having at least one, two or three modifications but not more than 20, 10 or 5 modifications of an amino acid sequence of SEQ ID NO:7 and/or an amino acid sequence of SEQ ID NO:9 or SEQ ID NO:10, or a sequence at least 95% identical (e.g., with 95-99% identity) to an amino acid sequence of SEQ ID NO:7 and/or an amino acid sequence of SEQ ID NO:9 or SEQ ID NO:10. In one embodiment, the intracellular signaling domain comprises the sequence of SEQ ID NO:7 and the sequence of SEQ ID NO:9 or SEQ ID NO:10, wherein the sequences comprising the intracellular signaling domain are expressed in the same frame and as a single polypeptide chain. In one embodiment, the nucleic acid sequence encoding the intracellular signaling domain comprises the nucleotide sequence of SEQ ID NO:18, or a sequence at least 95% identical (e.g., with 95-99% identity) thereof, and/or the CD3 zeta nucleotide sequence of SEQ ID NO:20 or SEQ ID NO:21, or a sequence at least 95% identical (e.g., with 95-99% identity) thereof.

In one embodiment, the intracellular signaling domain comprises a functional signaling domain of CD27 and/or a functional signaling domain of CD3 zeta. In one embodiment, the encoded intracellular signaling domain of CD27 comprises the amino acid sequence of SEQ ID NO:8 and/or the CD3 zeta amino acid sequence of SEQ ID NO:9 or SEQ ID NO:10. In one embodiment, the intracellular signaling domain comprises an amino acid sequence having at least one, two or three modifications but not more than 20, 10 or 5 modifications of an amino acid sequence of SEQ ID NO:8 and/or an amino acid sequence of SEQ ID NO:9 or SEQ ID NO:10, or a sequence at least 95% identical (e.g., with 95-99% identity) to an amino acid sequence of SEQ ID NO:8 and/or an amino acid sequence of SEQ ID NO:9 or SEQ ID NO:10. In one embodiment, the intracellular signaling domain comprises the sequence of SEQ ID NO:8 and the sequence of SEQ ID NO:9 or SEQ ID NO:10, wherein the sequences comprising the intracellular signaling domain are expressed in the same frame and as a single polypeptide chain. In one embodiment, the nucleic acid sequence encoding the intracellular signaling domain of CD27 comprises the nucleotide sequence of SEQ ID NO:19, or a sequence at least 95% identical (e.g., with 95-99% identity) thereof, and/or the CD3 zeta nucleotide sequence of SEQ ID NO:20 or SEQ ID NO:21, or a sequence at least 95% identical (e.g., with 95-99% identity) thereof.

In one embodiment, the intracellular signaling domain comprises a functional signaling domain of CD28 and/or a functional signaling domain of CD3 zeta. In one embodiment, the intracellular signaling domain of CD28 comprises the amino acid sequence of SEQ ID NO: 36 and/or the CD3 zeta amino acid sequence of SEQ ID NO:9 or SEQ ID NO:10. In one embodiment, the intracellular signaling domain comprises an amino acid sequence having at least one, two or three modifications but not more than 20, 10 or 5 modifications of an amino acid sequence of SEQ ID NO: 36 and/or an amino acid sequence of SEQ ID NO:9 or SEQ ID NO:10, or a sequence at least 95% identical (e.g., with 95-99% identity) to an amino acid sequence of SEQ ID NO: 36 and/or an amino acid sequence of SEQ ID NO:9 or SEQ ID NO:10. In one embodiment, the intracellular signaling domain comprises the sequence of SEQ ID NO: 36 and the sequence of SEQ ID NO:9 or SEQ ID NO:10, wherein the sequences comprising the intracellular signaling domain are expressed in the same frame and as a single polypeptide chain. In one embodiment, the nucleic acid sequence encoding the intracellular signaling domain of CD28 comprises the nucleotide sequence of SEQ ID NO: 37, or a sequence at least 95% identical (e.g., with 95-99% identity) thereof, and/or the CD3 zeta nucleotide sequence of SEQ ID NO:20 or SEQ ID NO:21, or a sequence at least 95% identical (e.g., with 95-99% identity) thereof.

In one embodiment, the intracellular signaling domain comprises a functional signaling domain of ICOS and/or a functional signaling domain of CD3 zeta. In one embodiment, the intracellular signaling domain of ICOS comprises the amino acid sequence of SEQ ID NO: 38 and/or the CD3 zeta amino acid sequence of SEQ ID NO:9 or SEQ ID NO:10. In one embodiment, the intracellular signaling domain comprises an amino acid sequence having at least one, two or three modifications but not more than 20, 10 or 5 modifications of an amino acid sequence of SEQ ID NO: 38 and/or an amino acid sequence of SEQ ID NO:9 or SEQ ID NO:10, or a sequence at least 95% identical (e.g., with 95-99% identity) to an amino acid sequence of SEQ ID NO: 38 and/or an amino acid sequence of SEQ ID NO:9 or SEQ ID NO:10. In one embodiment, the intracellular signaling domain comprises the sequence of SEQ ID NO: 38 and the sequence of SEQ ID NO:9 or SEQ ID NO:10, wherein the sequences comprising the intracellular signaling domain are expressed in the same frame and as a single polypeptide chain. In one embodiment, the nucleic acid sequence encoding the intracellular signaling domain of ICOS comprises the nucleotide sequence of SEQ ID NO: 44, or a sequence at least 95% identical (e.g., with 95-99% identity) thereof, and/or the CD3 zeta nucleotide sequence of SEQ ID NO:20 or SEQ ID NO:21, or a sequence at least 95% identical (e.g., with 95-99% identity) thereof.

In one embodiment, the CAR, e.g., CD19 CAR, further comprises a leader sequence comprising the amino acid sequence of SEQ ID NO:1.

### Exemplary CAR Molecules

In one embodiment, the CD19 CAR comprises the amino acid sequence of any of SEQ ID NO: 108; SEQ ID NO: 93; SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 111, SEQ ID NO: 114, or SEQ ID NO: 116. In one embodiment, the CD19 CAR comprises an amino acid sequence having at least one, two or three modifications but not more than 30, 20 or 10 modifications to any of SEQ ID NO: 108; SEQ ID NO: 93; SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 111, SEQ ID NO: 114, or SEQ ID NO: 116. In one embodiment, the CD19 CAR comprises an amino acid sequence at least 95% identical (e.g., with 95-99% identity) to any of SEQ ID NO: 108; SEQ ID NO: 93; SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 111, SEQ ID NO: 114, or SEQ ID NO: 116.

In an embodiment, the CAR molecule comprises a CD123 CAR described herein, e.g., a CD123 CAR described in US2014/0322212A1 or US2016/0068601A1, both incorporated herein by reference. In embodiments, the CD123 CAR comprises an amino acid, or has a nucleotide sequence shown in US2014/0322212A1 or US2016/0068601A1, both incorporated herein by reference.

In embodiments, the CAR molecule comprises a CD19 CAR molecule described herein, e.g., a CD19 CAR molecule described in US-2015-0283178-A1, e.g., CTL019. In embodiments, the CD19 CAR comprises an amino acid, or has a nucleotide sequence shown in US-2015-0283178-A1, incorporated herein by reference.

In one embodiment, CAR molecule comprises a BCMA CAR molecule described herein, e.g., a BCMA CAR described in US-2016-0046724-A1. In embodiments, the BCMA CAR comprises an amino acid, or has a nucleotide sequence shown in US-2016-0046724-A1, incorporated herein by reference.

In an embodiment, the CAR molecule comprises a CLL1 CAR described herein, e.g., a CLL1 CAR described in US2016/0051651A1, incorporated herein by reference. In embodiments, the CLL1 CAR comprises an amino acid, or has a nucleotide sequence shown in US2016/0051651A1, incorporated herein by reference.

In an embodiment, the CAR molecule comprises a CD33 CAR described herein, e.ga CD33 CAR described in US2016/0096892A1, incorporated herein by reference. In embodiments, the CD33 CAR comprises an amino acid, or has a nucleotide sequence shown in US2016/0096892A1, incorporated herein by reference.

In an embodiment, the CAR molecule comprises an EGFRvIII CAR molecule described herein, e.g., an EGFRvIII CAR described US2014/0322275A1, incorporated herein by reference. In embodiments, the EGFRvIII CAR comprises an amino acid, or has a nucleotide sequence shown in US2014/0322275A1, incorporated herein by reference.

In an embodiment, the CAR molecule comprises a mesothelin CAR described herein, e.g., a mesothelin CAR described in WO 2015/090230, incorporated herein by reference. In embodiments, the mesothelin CAR comprises an amino acid, or has a nucleotide sequence shown in WO 2015/090230, incorporated herein by reference.

In embodiments of any of the methods and compositions described herein, the cell comprising a CAR comprises a nucleic acid encoding the CAR.

In one embodiment, the nucleic acid encoding the CAR is a lentiviral vector. In one embodiment, the nucleic acid encoding the CAR is introduced into the cells by lentiviral transduction. In one embodiment, the nucleic acid encoding the CAR is an RNA, e.g., an in vitro transcribed RNA. In one embodiment, the nucleic acid encoding the CAR is introduced into the cells by electroporation.

In embodiments of any of the methods and compositions described herein, the cell is a T cell or an NK cell. In one embodiment, the T cell is an autologous or allogeneic T cell.

In one embodiment, the method further comprises administering an additional therapeutic agent for treating a disease described herein, e.g., an anti-cancer therapeutic agent. In embodiments, the method further comprises administering a lymphodepleting agent, e.g., described herein, e.g., before, concurrently with, or after administration with a CAR-expressing cell (e.g., CD19 CAR-expressing cell) and/or a PD-1 inhibitor described herein. In embodiments, the lymphodepleting agent comprises one or more chemotherapy agents, combination of chemotherapy agents, radiation therapy, or combination chemotherapy-radiation therapy, including, but not limited to, melphalan, cyclophosphamide, fludarabine, bendamustine, and cyclophosphamide-radiation therapy.

In embodiments of any of the methods and compositions described herein, the disease (e.g., cancer), e.g., the disease associated with CD19 expression, is a cancer. In one embodiment, the cancer is a hematological cancer. In embodiments, the hematological cancer is chosen from one or more of: B-cell acute lymphoid leukemia (BALL), T-cell acute lymphoid leukemia (TALL), small lymphocytic leukemia (SLL), acute lymphoid leukemia (ALL), chronic myelogenous leukemia (CML), chronic lymphocytic leukemia (CLL), mantle cell lymphoma (MCL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, or Waldenstrom macroglobulinemia. In embodiments, the hematological cancer is a leukemia, e.g., an acute leukemia or a chronic leukemia. In other embodiments, the hematological cancer is a lymphoma, e.g., non-Hodgkin lymphoma or Hodgkin lymphoma. In embodiments, the non-Hodgkin lymphoma is Burkitt lymphoma, chronic lymphocytic leukemia/small lymphocytic lymphoma (CLL/SLL), diffuse large B-cell lymphoma (DLBCL), follicular lymphoma, immunoblastic large cell lymphoma, precursor B-lymphoblastic lymphoma, and mantle cell lymphoma, mycosis fungoides, anaplastic large cell lymphoma, or precursor T-lymphoblastic lymphoma.

In one embodiment, the cancer expresses CD19, e.g., expresses CD19. In other embodiments, the cancer is relapsed or refractory B-ALL. In one embodiment, the cancer is relapsed or refractory B-ALL with lymph node involvement, e.g., with lymphomatous disease. In other embodiments, the cancer is DLBCL, e.g., relapsed or refractory DLBCL.

In some embodiments, the CAR therapy, e.g., a CD19 CAR therapy, is administered in combination with a PD-1 inhibitor, e.g., a PD-1 inhibitor as described herein, to a subject having Hodgkin Lymphoma (HL), e.g., relapsed or refractory HL. In an embodiment, the CAR therapy is administered to a subject having a relapsed and/or refractory HL after the PD-1 inhibitor. In another embodiment, the PD-1 inhibitor is administered to a subject having a relapsed and/or refractory HL after the CAR therapy, e.g., as described herein. In another embodiment, administration of the PD-1 inhibitor is initiated 20 days or less after administration of the CAR therapy, e.g., as described herein. In some embodiments, the CD19 CAR-expressing cell is a cell into which RNA encoding the CD19CAR was introduced, e.g., by electroporation. In embodiments, the subject comprises CD19-negative and CD19-positive cancer cells. In embodiments, the subject is treated with 6 doses of the CAR-expressing cells, e.g., over the course of 2 weeks. In embodiments, the dose comprises 1×10⁵-5×10⁶ or 8×10⁵-1.5×10⁶ CD19 CAR-expressing cells per dose, e.g., for subjects of <80 kg, or 1×10⁸ (±50%) or 1×10⁸ (±20%) CD19 CAR-expressing cells per dose, e.g., for subjecs of ≥80 kg. In embodiments, the dose comprises about 1×10⁵-1.5×10⁶ CD19 CAR-expressing cells per dose. In embodiments, the subject does not experience CRS or does not experience severe CRS. In embodiments, the subject experiences a complete response, partial response, or stable disease.

### Subjects

In one embodiment, the subject, e.g., the subject from which immune cells are acquired and/or the subject to be treated, is a human, e.g., a cancer patient. In certain embodiments, the subject is 18 years of age or younger (e.g., 18, 17, 16, 15, 14, 13, 12, 11, 10, 9, 8, 7, 6, 5, 4, 3, 2, 1 year or younger (e.g., 12 months, 6 months, 3 months or less)). In one embodiment, the subject is a pediatric cancer patient.

In other embodiments, the subject is an adult, e.g., the subject is older than 18 years of age (e.g., older than 18, 19, 20, 25, 30, 35, 40, 45, 50, 55, 60, 65, 70, 75, 80, or older). In one embodiment, the subject is an adult cancer patient.

In certain embodiments, the subject has a disease associated with expression of a tumor- or cancer associated-antigen, e.g., a disease as described herein. In one embodiment, the subject has a cancer, e.g., a cancer as described herein.

In one embodiment, the subject has a cancer that is chosen from a hematological cancer, a solid tumor, or a metastatic lesion thereof. Exemplary cancers include, but are not limited to, B-cell acute lymphocytic leukemia (B-ALL), T-cell acute lymphocytic leukemia (T-ALL), acute lymphocytic leukemia (ALL), chronic myelogenous leukemia (CML), chronic lymphocytic leukemia (CLL), B cell promyelocytic leukemia, blastic plasmacytoid dendritic cell neoplasm, Burkitt's lymphoma, diffuse large B cell lymphoma (DLBCL), follicular lymphoma, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin's lymphoma (NHL), Hodgkin's lymphoma (HL), plasmablastic lymphoma, plasmacytoid dendritic cell neoplasm, and Waldenstrom macroglobulinemia. In one embodiment, the cancer is ALL. In another embodiment, the cancer is CLL. In one embodiment, the cancer is DLBCL, e.g., relapsed or refractory DLBCL.

In embodiments, the subject has a leukemia, e.g., ALL (e.g., B-ALL). In embodiments, the subject has leukemia, e.g., ALL, and is a pediatric patient, e.g., is 18 years of age of younger (e.g., 18, 17, 16, 15, 14, 13, 12, 11, 10, 9, 8, 7, 6, 5, 4, 3, 2, 1 year or younger (e.g., 12 months, 6 months, 3 months or less)).

In embodiments, the subject has a lymphoma, e.g., DLBCL. In embodiments, the subject has lymphoma, e.g., DLBCL (e.g., relapsed or refractory DLBCL), and is an adult patient, e.g., is older than 18 years of age (e.g., older than 18, 19, 20, 25, 30, 35, 40, 45, 50, 55, 60, 65, 70, 75, 80, or older).

In embodiments, the subject has (e.g., is diagnosed with) a disease (e.g., cancer) described herein, e.g., a disease associated with CD19 expression, e.g., a cancer associated with CD19 expression described herein. In embodiments, the subject has a relapsed and/or refractory cancer, e.g., relapsed or refractory lymphoma, e.g., CD19+ lymphoma. In embodiments, the subject has DLBCL, e.g., CD19+ DLBCL. In embodiments, the subject has DLBCL transformed from follicular lymphoma. In embodiments, the subject has DLBCL and progressive lymphoma. In embodiments, the subject has DLBCL with primary mediastinal origin. In embodiments, the subject has previously been treated for a lymphoma, e.g., DLBCL, and has refractory lymphoma, e.g., refractory DLBCL.

In embodiments, the subject has (e.g., is diagnosed with) a high tumor burder cancer, e.g., before the first dose is administered. In one embodiment, the cancer is ALL or CLL. In embodiments, the subject has bone marrow blast levels of at least 1%, 2%, 3%, 4%, 5%, 6%, 7%, 8%, 9%, 10%, 15%, 20%, 25%, 30%, 35%, 40%, 45%, or 50%, e.g., at least 5%. In embodiments, the subject has a cancer in stage I, II, III, or IV. In embodiments, the subject has a tumor mass of at least 1, 2, 5, 10, 20, 50, 100, 200, 500, or 1000 g, e.g., in a single tumor or a plurality of tumors.

In embodiments, the subject has been administered a chemotherapy, e.g., a chemotherapy described herein (e.g., lymphodepleting chemotherapy, e.g., carboplatin and/or gemcitabine), prior to administration with a CAR-expressing cell and/or a PD-1 inhibitor described herein. In embodiments, the subject has been administered an immunotherapy, e.g., an allogeneic bone marrow transplant, prior to administration with a CAR-expressing cell and/or a PD-1 inhibitor described herein.

In embodiments of any of the methods and compositions described herein, the subject is a mammal, e.g., a human. In one embodiment, the subject expresses PD-1. In one embodiment, the cancer cell or a cell in close proximity to a cancer cell, e.g., a cancer-associated cell, in the subject expresses PD-1 or PL-L1. In an embodiment, the cancer-associated cell is an anti-tumor immune cell, e.g., a tumor infiltrating lymphocyte (TIL).

In one embodiment, the cell expressing a CAR, e.g., a CD19 CAR-expressing cell described herein, expresses PD-1.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting. Headings, sub-headings or numbered or lettered elements, e.g., (a), (b), (i) etc, are presented merely for ease of reading. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE DRAWINGS

FIG. 1A is an image of PD-L1 (CD274) expression in the patient's diffuse large B-cell lymphoma cells. Biopsy was obtained prior to CART19 cell infusion. Immunohistochemical staining with an anti-PD-L1 antibody from Cell Signaling (clone E1J2J, cat# 15165BF). The main image is at 40× magnification, the upper-right corner inset at 100×.

FIG. 1B is a panel of CT scans demonstrating clinical response to pembrolizumab after three weeks. Images on the left are on day of pembrolizumab infusion (day 26) and images on the right are 3 weeks after pembrolizumab infusion (day 45).

FIGS. 2A-2L are graphs showing correlative studies examining changes in T cell subsets in relation to CART19 infusion and pembrolizumab infusion. (FIG. 2A) Percentage of CART19+ CD3+ cells in peripheral blood. Percentage CART19+ of CD3+ cells prior to CART19 infusion (pre), three days after CART19 infusion (Day 3), 7 days after CART19 (Day 7), ten days after CART19 (Day 10), fourteen days after CART19 (Day 14), twenty-six days after CART19 and one hour after pembrolizumab infusion (Day 26), twenty-seven days after CART19 and 1 day after pembrolizumab (Day 27), twenty-eight days after CART19 and 2 days after pembrolizumab (Day 28), and forty-five days after CART19 and fourteen days after pembrolizumab (Day 45). (FIG. 2B) Fold change from baseline in IL-6 serum levels. (FIG. 2C) Percentage of PD1+CD4+ cells and PD1+CART19+CD4+ cells in peripheral blood. (FIG. 2D) Percentage of PD1+CD8+ cells and PD1+CART19+CD8+ cells in peripheral blood. (FIG. 2E) Percentage of PD1+Eomes+CD4+ cells and PD1+Eomes+CART19+CD4+ cells in peripheral blood. (FIG. 2F) Percentage of PD1+Eomes+CD8+ cells and PD1+Eomes+CART19+CD8+ cells in peripheral blood. (FIG. 2G) Percentage of Granzyme B+CD4+ cells and Granzyme B+CART19+CD4+ cells in peripheral blood. (FIG. 2H) Percentage of Granzyme B+CD8+ cells and Granzyme B+CART19+CD8+ cells in peripheral blood. (FIG. 2I) Percentage of PD1+CD4+ cells and PD1+Eomes+CD4+ cells in peripheral blood. (FIG. 2J) Percentage of PD1+CD4+CART19+ cells and PD1+Eomes+CD4+CART19+ cells in peripheral blood. (FIG. 2K) Percentage of PD1+CD8+ cells and PD1+Eomes+C8+ cells in peripheral blood. (FIG. 2L) Percentage of PD1+ CD8+CART19+ cells and PD1+Eomes+CD8+CART19+ cells in peripheral blood.

FIG. 3 shows the expression of PD-L1, PD1, LAG3, and TIM3 (from left to right in each set of four bars) in lymph node (LN) and bone marrow (BM) samples from five CR patients, one unclassified patient, and six PD patients.

FIGS. 4A, 4B, 4C, and 4D show flow cytometry analysis of PD1 and CAR19 expression on T cells. FIGS. 4A and 4B are representative flow cytometry profiles demonstrating the distribution of PD-1 and CAR19 expression on CD4+ T cells from subjects that are complete responders (CR) or non-responders (NR) to CART therapy. FIG. 4C is a graph showing the percent of PD1 cells in the CD4+ T cell population from groups of subjects with different responses to CART therapy. FIG. 4D is a graph showing the percent of PD1 cells in the CD8+ T cell population from groups of subjects with different responses to CART therapy.

FIGS. 5A and 5B show the distribution of PD1 expression in CD4 and CAR19-expressing cells (FIG. 5A) or CD8 and CAR19-expressing cells (FIG. 5B) from groups of subjects with different responses to CART therapy.

FIG. 6 shows flow cytometry analysis of PD1, CAR 19, LAG3, and TIM3 expression on T cells from subjects that are complete responders (CR) or non-responders (NR) to CART therapy.

FIGS. 7A and 7B show the distribution of PD1 and LAG3 expression (FIG. 7A) or PD1 and TIM3 expression (FIG. 7B) from groups of subjects with different responses to CART therapy.

FIG. 8 shows multiplex FIHC AQUA analysis showing significant difference between CD3+/PD-1+ cell populations in primary and secondary human DLBCL patient samples.

FIG. 9 shows AQUA analysis showing various levels of CD19 (lower panel) and PD-L1 (upper panel) in primary and secondary sites of DLBCL samples. A total of 40 human DLBCL patient samples, 25 primary and 15 secondary sites, were subjected to multiplex FIHC and followed by AQUA analysis to identify expression levels of CD19 and PD-L1 proteins.

FIG. 10 shows the percentage of CART19 cells in the patient from Case 3 after infusion of CART19 cells alone or after infusion of CART19 cells with a dose of Pembrolizumab.

FIG. 11 shows a graph of the probability of B cell recovery vs months post huCART19 infusion for patients receiving only huCART19 or huCART19 and Pembrolizumab.

FIG. 12 shows the percentage of CART19 in the patient from Case 6 infused with CART19 alone (circles) and after treatment with Pembrolizumab (squares).

FIG. 13 shows the percentage of CART19 in the patient from Case 6 with CART19 before and after treatment with Pembrolizumab, integrated with PET scan data before and after treatment with Pembrolizumab.

FIG. 14 is a graph depicting levels of CART19 RNA expression in the peripheral blood of four patients who received RNA CART19 therapy. Quantitative RT-PCR was performed on cells collected before and after each infusion (Days 0, 2, 4, 9, 11 and 14).

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR as described herein.

In one aspect, the stimulatory molecule is the zeta chain associated with the T cell receptor complex. In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from 4-1BB (i.e., CD137), CD27, ICOS, and/or CD28. In one aspect, the CAR comprises a chimeric fusion protein comprising an extracellular antigen binding domain, a transmembrane domain and an intracellular signaling domain comprising a functional signaling domain derived from a stimulatory molecule. In one aspect, the CAR comprises a chimeric fusion protein comprising an extracellular antigen binding domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen binding domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen binding domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen binding domain, wherein the leader sequence is optionally cleaved from the antigen recognition domain (e.g., a scFv) during cellular processing and localization of the CAR to the cellular membrane.

The term “signaling domain” refers to the functional portion of a protein which acts by transmitting information within the cell to regulate cellular activity via defined signaling pathways by generating second messengers or functioning as effectors by responding to such messengers. In some aspects, the signaling domain of the CAR described herein is derived from a stimulatory molecule or co-stimulatory molecule described herein, or is a synthesized or engineered signaling domain.

As used herein, the term “CD19” refers to the Cluster of Differentiation 19 protein, which is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_001178098. As used herein, “CD19” includes proteins comprising mutations, e.g., point mutations, fragments, insertions, deletions and splice variants of full length wild-type CD19. CD19 is expressed on most B lineage cancers, including, e.g., acute lymphoblastic leukemia, chronic lymphocyte leukemia and non-Hodgkin lymphoma. Other cells with express CD19 are provided below in the definition of “disease associated with expression of CD19.” It is also an early marker of B cell progenitors. See, e.g., Nicholson et al. Mol. Immun. 34 (16-17): 1157-1165 (1997). In one aspect the antigen-binding portion of the CART recognizes and binds an antigen within the extracellular domain of the CD19 protein. In one aspect, the CD19 protein is expressed on a cancer cell.

The term “antibody” or “antibody molecule” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules. In one embodiment, the antibody or antibody molecule comprises, e.g., consists of, an antibody fragment.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either VL or VH), camelid VHH domains, and multi-specific antibodies formed from antibody fragments such as a bivalent fragment comprising two Fab fragments linked by a disulfide brudge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3)(see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The portion of the CAR of the invention comprising an antibody or antibody fragment thereof may exist in a variety of forms where the antigen binding domain is expressed as part of a contiguous polypeptide chain including, for example, scFv antibody fragments, linear antibodies, single domain antibodies such as sdAb (either VL or VH), camelid VHH domains ,a humanized antibody, a bispecific antibody, an antibody conjugate (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR of the invention comprises an antibody fragment. In a further aspect, the CAR comprises an antibody fragment that comprises a scFv.

As used herein, the term “antibody molecule” refers to a protein, e.g., an immunoglobulin chain or fragment thereof, comprising at least one immunoglobulin variable domain sequence. The term antibody molecule encompasses antibodies and antibody fragments. In one embodiment, an antibody molecule encompasses a “binding domain” (also referred to herein as “anti-target (e.g., CD19) binding domain” or “target (e.g., CD19) binding domain”). In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species. The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The terms “cancer associated antigen” or “tumor antigen” or “proliferative disorder antigen” or “antigen associated with a proliferative disorder” interchangeably refers to a molecule (typically protein, carbohydrate or lipid) that is preferentially expressed on the surface of a cancer cell, either entirely or as a fragment (e.g., MHC/peptide), in comparison to a normal cell, and which is useful for the preferential targeting of a pharmacological agent to the cancer cell. In some embodiments, a tumor antigen is a marker expressed by both normal cells and cancer cells, e.g., a lineage marker, e.g., CD19 on B cells. In certain aspects, the tumor antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like. In some embodiments, the tumor antigen is an antigen that is common to a specific proliferative disorder. In some embodiments, a cancer-associated antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a cancer-associated antigen is a cell surface molecule that is inappropriately synthesized in the cancer cell, for instance, a molecule that contains deletions, additions or mutations in comparison to the molecule expressed on a normal cell. In some embodiments, a cancer-associated antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8 + T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Bood, 2011 117(16):4262-4272; Verma et al., J Immunol 2010 184(4):2156-2165; Willemsen et al., Gene Ther 2001 8(21) :1601-1608 ; Dao et al., Sci Transl Med 2013 5(176) :176ra33 ; Tassev et al., Cancer Gene Ther 2012 19(2):84-100). For example, TCR-like antibody can be identified from screening a library, such as a human scFv phage displayed library.

The phrase “disease associated with expression of CD19” includes, but is not limited to, a disease associated with expression of CD19 (e.g., wild-type or mutant CD19) or condition associated with cells which express, or at any time expressed, CD19 (e.g., wild-type or mutant CD19) including, e.g., proliferative diseases such as a cancer or malignancy or a precancerous condition such as a myelodysplasia, a myelodysplastic syndrome or a preleukemia; or a noncancer related indication associated with cells which express CD19. For the avoidance of doubt, a disease associated with expression of CD19 may include a condition associated with cells which do not presently express CD19, e.g., because CD19 expression has been downregulated, e.g., due to treatment with a molecule targeting CD19, e.g., a CD19 CAR, but which at one time expressed CD19. In one aspect, a cancer associated with expression of CD19 is a hematological cancer. In one aspect, the hematological cancer is a leukemia or a lymphoma. In one aspect, a cancer associated with expression of CD19 includes cancers and malignancies including, but not limited to, e.g., one or more acute leukemias including but not limited to, e.g., B-cell acute Lymphoid Leukemia (BALL), T-cell acute Lymphoid Leukemia (TALL), acute lymphoid leukemia (ALL); one or more chronic leukemias including but not limited to, e.g., chronic myelogenous leukemia (CML), Chronic Lymphoid Leukemia (CLL). Additional cancers or hematologic conditions associated with expression of CD19 comprise, but are not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma (MCL), Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further diseases associated with expression of CD19 expression include, but not limited to, e.g., atypical and/or non-classical cancers, malignancies, precancerous conditions or proliferative diseases associated with expression of CD19. Non-cancer related indications associated with expression of CD19 include, but are not limited to, e.g., autoimmune disease, (e.g., lupus), inflammatory disorders (allergy and asthma) and transplantation. In some embodiments, the CD19-expressing cells express, or at any time expressed, CD19 mRNA. In an embodiment, the CD19-expressing cells produce a CD19 protein (e.g., wild-type or mutant), and the CD19 protein may be present at normal levels or reduced levels. In an embodiment, the CD19-expressing cells produced detectable levels of a CD19 protein at one point, and subsequently produced substantially no detectable CD19 protein.

As used herein, the term “Programmed Death 1” or “PD-1” include isoforms, mammalian, e.g., human PD-1, species homologs of human PD-1, and analogs comprising at least one common epitope with PD-1. The amino acid sequence of PD-1, e.g., human PD-1, is known in the art, e.g., Shinohara T et al. (1994) Genomics 23(3):704-6; Finger L R, et al. Gene (1997) 197(1-2):177-87.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested, e.g., for the ability to bind CD19 using the functional assays described herein.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules, such as downregulation of TGF-β, and/or reorganization of cytoskeletal structures, and the like.

The term “stimulatory molecule,” refers to a molecule expressed by an immune effector cell (e.g., a T cell, NK cell, B cell) that provides the cytoplasmic signaling sequence(s) that regulate activation of the immune effector cell in a stimulatory way for at least some aspect of the immune effector cell signaling pathway, e.g., the T cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the invention includes, but is not limited to, those derived from CD3 zeta, common FcR gamma (FCER1G), Fc gamma RIIa, FcR beta (Fc epsilon R1b), CD3 gamma, CD3 delta , CD3 epsilon, CD5, CD22, CD79a, CD79b, CD278 (also known as “ICOS”), FcεRI, DAP10, DAP12, and CD66d. In a specific CAR of the invention, the intracellular signaling domain in any one or more CARs of the invention comprises an intracellular signaling sequence, e.g., a primary signaling sequence of CD3-zeta. In a specific CAR of the invention, the primary signaling sequence of CD3-zeta is the amino acid sequence provided as SEQ ID NO: 9, or the equivalent residues from a non-human species, e.g., mouse, rodent, monkey, ape and the like. In a specific CAR of the invention, the primary signaling sequence of CD3-zeta is the amino acid sequence as provided in SEQ ID NO: 10, or the equivalent residues from a non-human species, e.g., mouse, rodent, monkey, ape and the like.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. The intracellular signaling domain generates a signal that promotes an immune effector function of the CAR-expressingcell, e.g., a CART cell or CAR-expressing NK cell. Examples of immune effector function, e.g., in a CART cell or CAR-expressing NK cell, include cytolytic activity and helper activity, including the secretion of cytokines. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. In an embodiment, the intracellular signaling domain is synthesized or engineered. For example, in the case of a CAR-expressing immune effector cell, e.g., CART cell or CAR-expressing NK cell, a primary intracellular signaling domain can comprise a cytoplasmic sequence of a T cell receptor, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta, CD3 gamma, CD3 delta, CD3 epsilon, CDS, CD22, CD79a, CD79b, CD278 (“ICOS”), FcεRI CD66d, DAP10 and DAP12.

The term “zeta” or alternatively “zeta chain”, “CD3-zeta” or “TCR-zeta” is defined as the protein provided as GenBan Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof. In one aspect, the “zeta stimulatory domain” or a “CD3-zeta stimulatory domain” is the sequence provided as SEQ ID NO:10. In one aspect, the “zeta stimulatory domain” or a “CD3-zeta stimulatory domain” is the sequence provided as SEQ ID NO:9. Also encompassed herein are CD3 zeta domains comprising one or more mutations to the amino acid sequences described herein, e.g., SEQ ID NO: 9.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain can be the intracellular portion of a costimulatory molecule. The intracellular signaling domain can comprise the entire intracellular portion, or the entire native intracellular signaling domain, of the molecule from which it is derived, or a functional fragment thereof.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like. In one aspect, the “4-1BB costimulatory domain” is the sequence provided as SEQ ID NO:7 or the equivalent residues from a non-human species, e.g., mouse, rodent, monkey, ape and the like.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” is used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by its promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The term “humanized” refers to those forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise a significant portion of at least one, and typically two, variable domains, in which all or substantially all of the CDR regions correspond to those of a non-human immunoglobulin and all or substantially all of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

The term “fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1 (SEQ ID NO: 40). For example, n=1, n=2, n=3, n=4, n=5 and n=6, n=7, n=8, n=9 and n=10 (SEQ ID NO:41). In one embodiment, the flexible polypeptide linkers include, but are not limited to, (Gly₄ Ser)₄ (SEQ ID NO:27) or (Gly₄ Ser)₃ (SEQ ID NO:28). In another embodiment, the linkers include multiple repeats of (Gly₂Ser), (GlySer) or (Gly₃Ser) (SEQ ID NO:29). Also included within the scope of the invention are linkers described in WO2012/138475, incorporated herein by reference).

As used herein, a 5′ cap (also termed an RNA cap, an RNA 7-methylguanosine cap or an RNA m⁷G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000 (SEQ ID NO: 30), preferably greater than 64, more preferably greater than 100, most preferably greater than 300 or 400. poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

A dosage regimen, e.g., a therapeutic dosage regimen, can include one or more treatment intervals. The dosage regimen can result in at least one beneficial or desired clinical result including, but are not limited to, alleviation of a symptom, diminishment of extent of disease, stabilized (i.e., not worsening) state of disease, delay or slowing of disease progression, amelioration or palliation of the disease state, whether detectable or undetectable.

As used herein, a “treatment interval” refers to a treatment cycle, for example, a course of administration of a therapeutic agent that can be repeated, e.g., on a regular schedule. In embodiments, a dosage regimen can have one or more periods of no administration of the therapeutic agent in between treatment intervals. For example, a treatment interval can include one dose of a CAR molecule administered in combination with (prior, concurrently or after) administration of a second therapeutic agent, e.g., an inhibitor (e.g., a kinase inhibitor as described herein).

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human). In an embodiment, a subject is a mammal. In an embodiment, a subject is a human. In an embodiment, a subject is a patient. In one embodiment, the subject is a pedriatic subject. In other embodiments, the subject is an adult.

The term a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a binding partner (e.g., tumor antigen) protein present in a sample, but which antibody or ligand does not substantially recognize or bind other molecules in the sample.

“Regulatable chimeric antigen receptor (RCAR),”as used herein, refers to a set of polypeptides, typically two in the simplest embodiments, which when in an immune effector cell, provides the cell with specificity for a target cell, typically a cancer cell, and with regulatable intracellular signal generation. In some embodiments, an RCAR comprises at least an extracellular antigen binding domain, a transmembrane and a cytoplasmic signaling domain (also referred to herein as “an intracellular signaling domain”) comprising a functional signaling domain derived from a stimulatory molecule and/or costimulatory molecule as defined herein in the context of a CAR molecule. In some embodiments, the set of polypeptides in the RCAR are not contiguous with each other, e.g., are in different polypeptide chains. In some embodiments, the RCAR includes a dimerization switch that, upon the presence of a dimerization molecule, can couple the polypeptides to one another, e.g., can couple an antigen binding domain to an intracellular signaling domain. In some embodiments, the RCAR is expressed in a cell (e.g., an immune effector cell) as described herein, e.g., an RCAR-expressing cell (also referred to herein as “RCARX cell”). In an embodiment the RCARX cell is a T cell, and is referred to as a RCART cell. In an embodiment the RCARX cell is an NK cell, and is referred to as a RCARN cell. The RCAR can provide the RCAR-expressing cell with specificity for a target cell, typically a cancer cell, and with regulatable intracellular signal generation or proliferation, which can optimize an immune effector property of the RCAR-expressing cell. In embodiments, an RCAR cell relies at least in part, on an antigen binding domain to provide specificity to a target cell that comprises the antigen bound by the antigen binding domain.

“Membrane anchor” or “membrane tethering domain”, as that term is used herein, refers to a polypeptide or moiety, e.g., a myristoyl group, sufficient to anchor an extracellular or intracellular domain to the plasma membrane.

“Switch domain,” as that term is used herein, e.g., when referring to an RCAR, refers to an entity, typically a polypeptide-based entity, that, in the presence of a dimerization molecule, associates with another switch domain. The association results in a functional coupling of a first entity linked to, e.g., fused to, a first switch domain, and a second entity linked to, e.g., fused to, a second switch domain. A first and second switch domain are collectively referred to as a dimerization switch. In embodiments, the first and second switch domains are the same as one another, e.g., they are polypeptides having the same primary amino acid sequence, and are referred to collectively as a homodimerization switch. In embodiments, the first and second switch domains are different from one another, e.g., they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, e.g., FKBP or FRB-based, and the dimerization molecule is small molecule, e.g., a rapalogue. In embodiments, the switch domain is a polypeptide-based entity, e.g., an scFv that binds a myc peptide, and the dimerization molecule is a polypeptide, a fragment thereof, or a multimer of a polypeptide, e.g., a myc ligand or multimers of a myc ligand that bind to one or more myc scFvs. In embodiments, the switch domain is a polypeptide-based entity, e.g., myc receptor, and the dimerization molecule is an antibody or fragments thereof, e.g., myc antibody.

“Dimerization molecule,” as that term is used herein, e.g., when referring to an RCAR, refers to a molecule that promotes the association of a first switch domain with a second switch domain. In embodiments, the dimerization molecule does not naturally occur in the subject, or does not occur in concentrations that would result in significant dimerization. In embodiments, the dimerization molecule is a small molecule, e.g., rapamycin or a rapalogue, e.g, RAD001.

The term “bioequivalent” refers to an amount of an agent other than the reference compound (e.g., RAD001), required to produce an effect equivalent to the effect produced by the reference dose or reference amount of the reference compound (e.g., RAD001). In an embodiment the effect is the level of mTOR inhibition, e.g., as measured by P70 S6 kinase inhibition, e.g., as evaluated in an in vivo or in vitro assay, e.g., as measured by an assay described herein, e.g., the Boulay assay, or measurement of phosphorylated S6 levels by western blot. In an embodiment, the effect is alteration of the ratio of PD-1 positive/PD-1 negative T cells, as measured by cell sorting. In an embodiment a bioequivalent amount or dose of an mTOR inhibitor is the amount or dose that achieves the same level of P70 S6 kinase inhibition as does the reference dose or reference amount of a reference compound. In an embodiment, a bioequivalent amount or dose of an mTOR inhibitor is the amount or dose that achieves the same level of alteration in the ratio of PD-1 positive/PD-1 negative T cells as does the reference dose or reference amount of a reference compound.

The term “low, immune enhancing, dose” when used in conjuction with an mTOR inhibitor, e.g., an allosteric mTOR inhibitor, e.g., RAD001 or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In an embodiment, the low, immune enhancing, dose of mTOR inhibitor results in a decrease in the number of PD-1 positive T cells and/or an increase in the number of PD-1 negative T cells, or an increase in the ratio of PD-1 negative T cells/PD-1 positive T cells. In an embodiment, the low, immune enhancing, dose of mTOR inhibitor results in an increase in the number of naive T cells. In an embodiment, the low, immune enhancing, dose of mTOR inhibitor results in one or more of the following:

an increase in the expression of one or more of the following markers: CD62L^{high}, CD127^{high}, CD27⁺, and BCL2, e.g., on memory T cells, e.g., memory T cell precursors;

a decrease in the expression of KLRG1, e.g., on memory T cells, e.g., memory T cell precursors; and

an increase in the number of memory T cell precursors, e.g., cells with any one or combination of the following characteristics: increased CD62L^{high}, increased CD127^{high}, increased CD27⁺, decreased KLRG1, and increased BCL2;

wherein any of the changes described above occurs, e.g., at least transiently, e.g., as compared to a non-treated subject.

“Progressive” as used herein refers to a disease, e.g., cancer, that is progressing or worsening. With solid tumors, e.g., lung cancer, progressive disease typically shows at least 20% growth in size or the tumor or spread of the tumor since the beginning of treatment.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” or “relapse” as used herein refers to the return or reappearance of a disease (e.g., cancer) or the signs and symptoms of a disease such as cancer after a period of improvement or responsiveness, e.g., after prior treatment of a therapy, e.g., cancer therapy. The initial period of responsiveness may involve the level of cancer cells falling below a certain threshold, e.g., below 20%, 1%, 10%, 5%, 4%, 3%, 2%, or 1%. The reappearance may involve the level of cancer cells rising above a certain threshold, e.g., above 20%, 1%, 10%, 5%, 4%, 3%, 2%, or 1%. For example, e.g., in the context of B-ALL, the reappearance may involve, e.g., a reappearance of blasts in the blood, bone marrow (>5%), or any extramedullary site, after a complete response. A complete response, in this context, may involve <5% BM blast. More generally, in an embodiment, a response (e.g., complete response or partial response) can involve the absence of detectable MRD (minimal residual disease). In an embodiment, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

A “complete response” or “CR” refers to the absence of detectable evidence of disease, e.g., cancer, e.g., a complete remission, to a treatment. A complete response may be identified, e.g., using the NCCN Guidelines®, or Cheson et al, J Clin Oncol 17:1244 (1999) and Cheson et al., “Revised Response Criteria for Malignant Lymphoma”, J Clin Oncol 25:579-586 (2007) (both of which are incorporated by reference herein in their entireties), as described herein. For example, in the context of B-ALL, a complete response may involve <5% BM blasts.

A “complete responder” as used herein refers to a subject having a disease, e.g., a cancer, who exhibits a complete response, e.g., a complete remission, to a treatment.

A “partial response” or “PR” refers to a decrease in the disease, e.g., cancer, although, e.g., there is still detectable disease present.

A “partial responder” as used herein refers to a subject having a disease, e.g., a cancer, who exhibits a partial response, e.g., a partial remission, to a treatment. A partial response may be identified, e.g., using the NCCN Guidelines®, or Cheson criteria as described herein.

A “non-responder” as used herein refers to a subject having a disease, e.g., a cancer, who does not exhibit a response to a treatment, e.g., the patient has stable disease or progressive disease after administration of a treatment, e.g., a treatment described herein. A non-responder may be identified, e.g., using the NCCN Guidelines®, or Cheson criteria as described herein.

Several methods can be used to determine if a patient responds to a treatment including, for example, criteria provided by NCCN Clinical Practice Guidelines in Oncology (NCCN Guidelines®). For example, in the context of B-ALL, a complete response or complete responder, may involve one or more of: <5% BM blast, >1000 neutrophil/ANC (/μL). >100,000 platelets (/μL) with no circulating blasts or extramedullary disease (No lymphadenopathy, splenomegaly, skin/gum infiltration/testicular mass/CNS involvement), Trilineage hematopoiesis, and no recurrence for 4 weeks. A partial responder may involve one or more of ≥50% reduction in BM blast, >1000 neutrophil/ANC (/μL). >100,000 platelets (/μL). A non-responder can show disease progression, e.g., >25% in BM blasts.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

### Description

Provided herein are compositions and methods for treating a disease such as cancer, by administering a cell comprising a chimeric antigen receptor that targets an antigen, e.g., antigen described herein, e.g., CD19, e.g., CD19 CAR, in combination with a PD-1 inhibitor. Exemplary components to generate a CAR molecule, e.g., CD19 CAR and a CAR-expressing cell (e.g., CD19 CAR-expressing cell) are disclosure herein. Exemplary PD-1 inhibitors are also described herein.

In embodiments, the combination therapy of a CAR-expressing cell (e.g., CD19 CAR-expressing cell) described herein and a PD-1 inhibitor described herein results in one or more of the following: improved or increased anti-tumor activity of the CAR-expressing cell; increased proliferation or persistence of the CAR-expressing cell; improved or increased infiltration of the CAR-expressing cell; improved inhibition of tumor progression; delay of tumor progression; inhibition or reduction in cancer cell proliferation; and/or reduction in tumor burden, e.g., tumor volume, or size. In an embodiment, the combination therapy of a CD19 CAR-expressing cell, e.g., a plurality of CD19 CAR-expressing cells, and a PD-1 inhibitor described herein results in increased or improved persistence of a CD19 CAR-expressing cell, e.g., increased or improved persistence of a plurality of CD19 CAR-expressing cells.

In some embodiments, administration of the PD-1 inhibitor prior to or subsequent to administration of a CAR-expressing cell (e.g., CD19 CAR-expressing cell) results in increased therapeutic efficacy, e.g., increased inhibition of tumor progression and/or tumor growth, in some cancers, e.g., as compared to administration og the PD-1 inhibitor or CAR-expressing cell alone.

PD-1 is known to downregulate the immune response, e.g., anti-tumor immune response. PD-1 and/or PD-L1 can also be expressed by cancer cells or cancer associated cells, e.g., tumor infiltrating lymphocytes (TILs). Without wishing to be bound by theory, in some embodiments, a subject that is administered the combination therapy described herein, e.g., a CAR-expressing cell (e.g., CD19 CAR-expressing cell) and a PD-1 inhibitor, is more likely to have increased anti-tumor activity if the subject has one or more of: a cancer that expresses, e.g., highly expresses, PD-1 and/or PD-L1; a cancer that is infiltrated by anti-tumor immune cells, e.g., tumor infiltrating lymphocytes (TILs); and/or cancer-associated cells that express, e.g., highly express, PD-1 and/or PD-L1, as compared to a subject that is not administered the combination therapy, or is administered a CAR-expressing cell or PD-1 inhibitor alone. For example, without wishing to be bound by theory, treatment with a PD-1 inhibitor prevents or reduces the downregulation of the anti-tumor immune response, e.g., exhaustion of anti-tumor immune cells, e.g., TILs, thereby increasing the anti-tumor efficacy of the CAR-expressing cell. Without wishing to be bound by theory, administration of the combination therapy, e.g., a CAR-expressing cell, e.g., a CD19 CAR-expressing cell, and an immune checkpoint inhibitor, e.g., a PD-linhibitor, can reduce exhaustion of T cells leading to improved, e.g., longer, persistence of CAR-expressing cells. In an embodiment, administration of a combination of a CD19 CAR-expressing cell and a PD-1 inhibitor can result in improved, e.g., longer, persistence of CD19 CAR-expressing cells.

### Chimeric Antigen Receptor (CAR)

The present disclosure encompasses immune effector cells (e.g., T cells or NK cells) comprising a CAR molecule that targets, e.g., specifically binds, to an antigen, e.g., antigen described herein, e.g., CD19 (a CAR, e.g., CD19 CAR). In one embodiment, the immune effector cells are engineered to express the CAR, e.g., CD19 CAR. In one embodiment, the immune effector cells comprise a recombinant nucleic acid construct comprising nucleic acid sequences encoding the CAR, e.g., CD19 CAR.

In embodiments, the CAR, e.g., CD19 CAR, comprises an antigen binding domain that specifically binds to an antigen, e.g., CD19, e.g., antigen binding domain (e.g., CD19 binding domain), a transmembrane domain, and an intracellular signaling domain. In one embodiment, the sequence of the antigen binding domain is contiguous with and in the same reading frame as a nucleic acid sequence encoding an intracellular signaling domain. The intracellular signaling domain can comprise a costimulatory signaling domain and/or a primary signaling domain, e.g., a zeta chain. The costimulatory signaling domain refers to a portion of the CAR comprising at least a portion of the intracellular domain of a costimulatory molecule.

Sequences of non-limiting examples of various components that can be part of a CAR molecule (e.g., CD19 CAR molecule) described herein, are listed in Table 1, where “aa” stands for amino acids, and “na” stands for nucleic acids that encode the corresponding peptide.

In accordance with any method or composition described herein, in embodiments, a CAR molecule comprises a CD123 CAR described herein, e.g., a CD123 CAR described in US2014/0322212A1 or US2016/0068601A1, both incorporated herein by reference. In embodiments, the CD123 CAR comprises an amino acid, or has a nucleotide sequence shown in US2014/0322212A1 or US2016/0068601A1, both incorporated herein by reference. In other embodiments, a CAR molecule comprises a CD19 CAR molecule described herein, e.g., a CD19 CAR molecule described in US-2015-0283178-A1, e.g., CTL019. In embodiments, the CD19 CAR comprises an amino acid, or has a nucleotide sequence shown in US-2015-0283178-A1, incorporated herein by reference. In one embodiment, CAR molecule comprises a BCMA CAR molecule described herein, e.g., a BCMA CAR described in US-2016-0046724-A1. In embodiments, the BCMA CAR comprises an amino acid, or has a nucleotide sequence shown in US-2016-0046724-A1, incorporated herein by reference. In an embodiment, the CAR molecule comprises a CLL1 CAR described herein, e.g., a CLL1 CAR described in US2016/0051651A1, incorporated herein by reference. In embodiments, the CLL1 CAR comprises an amino acid, or has a nucleotide sequence shown in US2016/0051651A1, incorporated herein by reference. In an embodiment, the CAR molecule comprises a CD33 CAR described herein, e.ga CD33 CAR described in US2016/0096892A1, incorporated herein by reference. In embodiments, the CD33 CAR comprises an amino acid, or has a nucleotide sequence shown in US2016/0096892A1, incorporated herein by reference. In an embodiment, the CAR molecule comprises an EGFRvIII CAR molecule described herein, e.g., an EGFRvIII CAR described US2014/0322275A1, incorporated herein by reference. In embodiments, the EGFRvIII CAR comprises an amino acid, or has a nucleotide sequence shown in US2014/0322275A1, incorporated herein by reference. In an embodiment, the CAR molecule comprises a mesothelin CAR described herein, e.g., a mesothelin CAR described in WO 2015/090230, incorporated herein by reference. In embodiments, the mesothelin CAR comprises an amino acid, or has a nucleotide sequence shown in WO 2015/090230, incorporated herein by reference.

TABLE 1
Sequences of various components of CAR
(aa-amino acid sequence, na-nucleic acid sequence)

| SEQ | | |
|---|---|---|
| ID | | |
| NO | Descrip. | Sequence |
| 11 | EF-1 promoter | CGTGAGGCTCCGGTGCCCGTCAGTGGGCAGAGCGCACATCGCCCAC |
| | (na) | AGTCCCCGAGAAGTTGGGGGGAGGGGTCGGCAATTGAACCGGTGCC |
| | | TAGAGAAGGTGGCGCGGGGTAAACTGGGAAAGTGATGTCGTGTACT |
| | | GGCTCCGCCTTTTTCCCGAGGGTGGGGGAGAACCGTATATAAGTGCA |
| | | GTAGTCGCCGTGAACGTTCTTTTTCGCAACGGGTTTGCCGCCAGAAC |
| | | ACAGGTAAGTGCCGTGTGTGGTTCCCGCGGGCCTGGCCTCTTTACGG |
| | | GTTATGGCCCTTGCGTGCCTTGAATTACTTCCACCTGGCTGCAGTACG |
| | | TGATTCTTGATCCCGAGCTTCGGGTTGGAAGTGGGTGGGAGAGTTCG |
| | | AGGCCTTGCGCTTAAGGAGCCCCTTCGCCTCGTGCTTGAGTTGAGGC |
| | | CTGGCCTGGGCGCTGGGGCCGCCGCGTGCGAATCTGGTGGCACCTTC |
| | | GCGCCTGTCTCGCTGCTTTCGATAAGTCTCTAGCCATTTAAAATTTTT |
| | | GATGACCTGCTGCGACGCTTTTTTTCTGGCAAGATAGTCTTGTAAAT |
| | | GCGGGCCAAGATCTGCACACTGGTATTTCGGTTTTTGGGGCCGCGGG |
| | | CGGCGACGGGGCCCGTGCGTCCCAGCGCACATGTTCGGCGAGGCGG |
| | | GGCCTGCGAGCGCGGCCACCGAGAATCGGACGGGGGTAGTCTCAAG |
| | | CTGGCCGGCCTGCTCTGGTGCCTGGCCTCGCGCCGCCGTGTATCGCC |
| | | CCGCCCTGGGCGGCAAGGCTGGCCCGGTCGGCACCAGTTGCGTGAG |
| | | CGGAAAGATGGCCGCTTCCCGGCCCTGCTGCAGGGAGCTCAAAATG |
| | | GAGGACGCGGCGCTCGGGAGAGCGGGCGGGTGAGTCACCCACACAA |
| | | AGGAAAAGGGCCTTTCCGTCCTCAGCCGTCGCTTCATGTGACTCCAC |
| | | GGAGTACCGGGCGCCGTCCAGGCACCTCGATTAGTTCTCGAGCTTTT |
| | | GGAGTACGTCGTCTTTAGGTTGGGGGGAGGGGTTTTATGCGATGGAG |
| | | TTTCCCCACACTGAGTGGGTGGAGACTGAAGTTAGGCCAGCTTGGCA |
| | | CTTGATGTAATTCTCCTTGGAATTTGCCCTTTTTGAGTTTGGATCTTG |
| | | GTTCATTCTCAAGCCTCAGACAGTGGTTCAAAGTTTTTTTCTTCCATT |
| | | TCAGGTGTCGTGA |
| 1 | Leader (aa) | MALPVTALLLPLALLLHAARP |
| 12 | Leader (na) | ATGGCCCTGCCTGTGACAGCCCTGCTGCTGCCTCTGGCTCTGCTGCTG |
| | | CATGCCGCTAGACCC |
| 290 | Leader codon | ATGGCCCTCCCTGTCACCGCCCTGCTGCTTCCGCTGGCTCTTCTGCTC |
| | optimized (na) | CACGCCGCTCGGCCC |
| 2 | CD 8 hinge (aa) | TTTPAPRPPTPAPTIASQPLSLRPEACRPAAGGAVHTRGLDFACD |
| 13 | CD8 hinge (na) | ACCACGACGCCAGCGCCGCGACCACCAACACCGGCGCCCACCATCG |
| | | CGTCGCAGCCCCTGTCCCTGCGCCCAGAGGCGTGCCGGCCAGCGGCG |
| | | GGGGGCGCAGTGCACACGAGGGGGCTGGACTTCGCCTGTGAT |
| 3 | Ig4 hinge (aa) | ESKYGPPCPPCPAPEFLGGPSVFLFPPKPKDTLMISRTPEVTCVVVDVSQ |
| | | EDPEVQFNWYVDGVEVHNAKTKPREEQFNSTYRVVSVLTVLHQDWLN |
| | | GKEYKCKVSNKGLPSSIEKTISKAKGQPREPQVYTLPPSQEEMTKNQVS |
| | | LTCLVKGFYPSDIAVEWESNGQPENNYKTTPPVLDSDGSFFLYSRLTVD |
| | | KSRWQEGNVFSCSVMHEALHNHYTQKSLSLSLGKM |
| 14 | Ig4 hinge (na) | GAGAGCAAGTACGGCCCTCCCTGCCCCCCTTGCCCTGCCCCCGAGTT |
| | | CCTGGGCGGACCCAGCGTGTTCCTGTTCCCCCCCAAGCCCAAGGACA |
| | | CCCTGATGATCAGCCGGACCCCCGAGGTGACCTGTGTGGTGGTGGAC |
| | | GTGTCCCAGGAGGACCCCGAGGTCCAGTTCAACTGGTACGTGGACG |
| | | GCGTGGAGGTGCACAACGCCAAGACCAAGCCCCGGGAGGAGCAGTT |
| | | CAATAGCACCTACCGGGTGGTGTCCGTGCTGACCGTGCTGCACCAGG |
| | | ACTGGCTGAACGGCAAGGAATACAAGTGTAAGGTGTCCAACAAGGG |
| | | CCTGCCCAGCAGCATCGAGAAAACCATCAGCAAGGCCAAGGGCCAG |
| | | CCTCGGGAGCCCCAGGTGTACACCCTGCCCCCTAGCCAAGAGGAGA |
| | | TGACCAAGAACCAGGTGTCCCTGACCTGCCTGGTGAAGGGCTTCTAC |
| | | CCCAGCGACATCGCCGTGGAGTGGGAGAGCAACGGCCAGCCCGAGA |
| | | ACAACTACAAGACCACCCCCCCTGTGCTGGACAGCGACGGCAGCTTC |
| | | TTCCTGTACAGCCGGCTGACCGTGGACAAGAGCCGGTGGCAGGAGG |
| | | GCAACGTCTTTAGCTGCTCCGTGATGCACGAGGCCCTGCACAACCAC |
| | | TACACCCAGAAGAGCCTGAGCCTGTCCCTGGGCAAGATG |
| 4 | IgD hinge (aa) | RWPESPKAQASSVPTAQPQAEGSLAKATTAPATTRNTGRGGEEKKKEK |
| | | EKEEQEERETKTPECPSHTQPLGVYLLTPAVQDLWLRDKATFTCFVVGS |
| | | DLKDAHLTWEVAGKVPTGGVEEGLLERHSNGSQSQHSRLTLPRSLWNA |
| | | GTSVTCTLNHPSLPPQRLMALREPAAQAPVKLSLNLLASSDPPEAASWL |
| | | LCEVSGFSPPNILLMWLEDQREVNTSGFAPARPPPQPGSTTFWAWSVLR |
| | | VPAPPSPQPATYTCVVSHEDSRTLLNASRSLEVSYVTDH |
| 15 | IgD hinge (na) | AGGTGGCCCGAAAGTCCCAAGGCCCAGGCATCTAGTGTTCCTACTGC |
| | | ACAGCCCCAGGCAGAAGGCAGCCTAGCCAAAGCTACTACTGCACCT |
| | | GCCACTACGCGCAATACTGGCCGTGGCGGGGAGGAGAAGAAAAAGG |
| | | AGAAAGAGAAAGAAGAACAGGAAGAGAGGGAGACCAAGACCCCTG |
| | | AATGTCCATCCCATACCCAGCCGCTGGGCGTCTATCTCTTGACTCCC |
| | | GCAGTACAGGACTTGTGGCTTAGAGATAAGGCCACCTTTACATGTTT |
| | | CGTCGTGGGCTCTGACCTGAAGGATGCCCATTTGACTTGGGAGGTTG |
| | | CCGGAAAGGTACCCACAGGGGGGGTTGAGGAAGGGTTGCTGGAGCG |
| | | CCATTCCAATGGCTCTCAGAGCCAGCACTCAAGACTCACCCTTCCGA |
| | | GATCCCTGTGGAACGCCGGGACCTCTGTCACATGTACTCTAAATCAT |
| | | CCTAGCCTGCCCCCACAGCGTCTGATGGCCCTTAGAGAGCCAGCCGC |
| | | CCAGGCACCAGTTAAGCTTAGCCTGAATCTGCTCGCCAGTAGTGATC |
| | | CCCCAGAGGCCGCCAGCTGGCTCTTATGCGAAGTGTCCGGCTTTAGC |
| | | CCGCCCAACATCTTGCTCATGTGGCTGGAGGACCAGCGAGAAGTGA |
| | | ACACCAGCGGCTTCGCTCCAGCCCGGCCCCCACCCCAGCCGGGTTCT |
| | | ACCACATTCTGGGCCTGGAGTGTCTTAAGGGTCCCAGCACCACCTAG |
| | | CCCCCAGCCAGCCACATACACCTGTGTTGTGTCCCATGAAGATAGCA |
| | | GGACCCTGCTAAATGCTTCTAGGAGTCTGGAGGTTTCCTACGTGACT |
| | | GACCATT |
| 6 | CD8 | IYIWAPLAGTCGVLLLSLVITLYC |
| | Transmembrane | |
| | (aa) | |
| 17 | CD8 | ATCTACATCTGGGCGCCCTTGGCCGGGACTTGTGGGGTCCTTCTCCT |
| | Transmembrane | GTCACTGGTTATCACCCTTTACTGC |
| | (na) | |
| 291 | CD8 | ATCTACATTTGGGCCCCTCTGGCTGGTACTTGCGGGGTCCTGCTGCTT |
| | Transmembrane, | TCACTCGTGATCACTCTTTACTGT |
| | codon optimized | |
| | (na) | |
| 7 | 4-1BB | KRGRKKLLYIFKQPFMRPVQTTQEEDGCSCRFPEEEEGGCEL |
| | intracellular | |
| | domain (aa) | |
| 18 | 4-1BB | AAACGGGGCAGAAAGAAACTCCTGTATATATTCAAACAACCATTTAT |
| | intracellular | GAGACCAGTACAAACTACTCAAGAGGAAGATGGCTGTAGCTGCCGA |
| | domain (na) | TTTCCAGAAGAAGAAGAAGGAGGATGTGAACTG |
| 292 | 4-1BB | AAGCGCGGTCGGAAGAAGCTGCTGTACATCTTTAAGCAACCCTTCAT |
| | intracellular | GAGGCCTGTGCAGACTACTCAAGAGGAGGACGGCTGTTCATGCCGG |
| | domain, codon | TTCCCAGAGGAGGAGGAAGGCGGCTGCGAACTG |
| | optimized (na) | |
| 8 | CD27 (aa) | QRRKYRSNKGESPVEPAEPCRYSCPREEEGSTIPIQEDYRKPEPACSP |
| 19 | CD27 (na) | AGGAGTAAGAGGAGCAGGCTCCTGCACAGTGACTACATGAACATGA |
| | | CTCCCCGCCGCCCCGGGCCCACCCGCAAGCATTACCAGCCCTATGCC |
| | | CCACCACGCGACTTCGCAGCCTATCGCTCC |
| 9 | CD3-zeta (aa) | RVKFSRSADAPAYKQGQNQLYNELNLGRREEYDVLDKRRGRDPEMGG |
| | (Q/K mutant) | KPRRKNPQEGLYNELQKDKMAEAYSEIGMKGERRRGKGHDGLYQGLS |
| | | TATKDTYDALHMQALPPR |
| 20 | CD3-zeta (na) | AGAGTGAAGTTCAGCAGGAGCGCAGACGCCCCCGCGTACAAGCAGG |
| | (Q/K mutant) | GCCAGAACCAGCTCTATAACGAGCTCAATCTAGGACGAAGAGAGGA |
| | | GTACGATGTTTTGGACAAGAGACGTGGCCGGGACCCTGAGATGGGG |
| | | GGAAAGCCGAGAAGGAAGAACCCTCAGGAAGGCCTGTACAATGAAC |
| | | TGCAGAAAGATAAGATGGCGGAGGCCTACAGTGAGATTGGGATGAA |
| | | AGGCGAGCGCCGGAGGGGCAAGGGGCACGATGGCCTTTACCAGGGT |
| | | CTCAGTACAGCCACCAAGGACACCTACGACGCCCTTCACATGCAGGC |
| | | CCTGCCCCCTCGC |
| 293 | CD3-zeta, codon | CGCGTGAAATTCAGCCGCAGCGCAGATGCTCCAGCCTACAAGCAGG |
| | optimized (na) | GGCAGAACCAGCTCTACAACGAACTCAATCTTGGTCGGAGAGAGGA |
| | (Q/K mutant) | GTACGACGTGCTGGACAAGCGGAGAGGACGGGACCCAGAAATGGGC |
| | | GGGAAGCCGCGCAGAAAGAATCCCCAAGAGGGCCTGTACAACGAGC |
| | | TCCAAAAGGATAAGATGGCAGAAGCCTATAGCGAGATTGGTATGAA |
| | | AGGGGAACGCAGAAGAGGCAAAGGCCACGACGGACTGTACCAGGG |
| | | ACTCAGCACCGCCACCAAGGACACCTATGACGCTCTTCACATGCAGG |
| | | CCCTGCCGCCTCGG |
| 10 | CD3-zeta (aa) | RVKFSRSADAPAYQQGQNQLYNELNLGRREEYDVLDKRRGRDPEMGG |
| | (NCBI | KPRRKNPQEGLYNELQKDKMAEAYSEIGMKGERRRGKGHDGLYQGLS |
| | Reference | TATKDTYDALHMQALPPR |
| | Sequence | |
| | NM_000734.3) | |
| 21 | CD3-zeta (na) | AGAGTGAAGTTCAGCAGGAGCGCAGACGCCCCCGCGTACCAGCAGG |
| | (NCBI | GCCAGAACCAGCTCTATAACGAGCTCAATCTAGGACGAAGAGAGGA |
| | Reference | GTACGATGTTTTGGACAAGAGACGTGGCCGGGACCCTGAGATGGGG |
| | Sequence | GGAAAGCCGAGAAGGAAGAACCCTCAGGAAGGCCTGTACAATGAAC |
| | NM_000734.3) | TGCAGAAAGATAAGATGGCGGAGGCCTACAGTGAGATTGGGATGAA |
| | | AGGCGAGCGCCGGAGGGGCAAGGGGCACGATGGCCTTTACCAGGGT |
| | | CTCAGTACAGCCACCAAGGACACCTACGACGCCCTTCACATGCAGGC |
| | | CCTGCCCCCTCGC |
| 36 | CD28 | RSKRSRLLHSDYMNMTPRRPGPTRKHYQPYAPPRDFAAYRS |
| | | Intracellular |
| | | domain (amino |
| | | acid sequence) |
| 37 | CD28 | AGGAGTAAGAGGAGCAGGCTCCTGCACAGTGACTACATGAACATGA |
| | Intracellular | CTCCCCGCCGCCCCGGGCCCACCCGCAAGCATTACCAGCCCTATGCC |
| | domain | CCACCACGCGACTTCGCAGCCTATCGCTCC |
| | (nucleotide | |
| | sequence) | |
| 38 | ICOS | TKKKYSSSVHDPNGEYMFMRAVNTAKKSRLTDVTL |
| | Intracellular | |
| | domain (amino | |
| | acid sequence) | |
| 43 | Y to F mutant | TKKKYSSSVHDPNGEFMFMRAVNTAKKSRLTDVTL |
| | ICOS domain | |
| | (aa) | |
| 44 | ICOS | ACAAAAAAGAAGTATTCATCCAGTGTGCACGACCCTAACGGTGAAT |
| | Intracellular | ACATGTTCATGAGAGCAGTGAACACAGCCAAAAAATCCAGACTCAC |
| | domain | AGATGTGACCCTA |
| | (nucleotide | |
| | sequence) | |
| 5 | GS hinge/linker | GGGGSGGGGS |
| | (aa) | |
| 16 | GS hinge/linker | GGTGGCGGAGGTTCTGGAGGTGGAGGTTCC |
| | (na) | |
| 39 | GS hinge/linker | GGTGGCGGAGGTTCTGGAGGTGGGGGTTCC |
| | (na) | |
| 25 | linker | GGGGS |
| 26 | linker | (Gly-Gly-Gly-Gly-Ser)n, where n = 1-6, e.g., GGGGSGGGGS |
| | | GGGGSGGGGS GGGGSGGGGS |
| 27 | linker | (Gly4 Ser)4 |
| 28 | linker | (Gly4 Ser)3 |
| 29 | linker | (Gly3Ser) |
| 40 | linker | (Gly-Gly-Gly-Ser)n where n is a positive integer equal to or greater than 1 |
| 41 | linker | (Gly-Gly-Gly-Ser)n, where n = 1-10, e.g., GGGSGGGSGG GSGGGSGGGS |
| | | GGGSGGGSGG GSGGGSGGGS |
| 42 | linker | GSTSGSGKPGSGEGSTKG |
| 30 | polyA | (A)₅₀₀₀ |
| | | This sequence may encompass 50-5000 adenines. |
| 31 | polyT | (T)₁₀₀ |
| 32 | polyT | (T)₅₀₀₀ |
| | | This sequence may encompass 50-5000 thymines. |
| 33 | polyA | (A)₅₀₀₀ |
| | | This sequence may encompass 100-5000 adenines. |
| 34 | polyA | (A)₄₀₀ |
| | | This sequence may encompass 100-400 adenines. |
| 35 | polyA | (A)₂₀₀₀ |
| | | This sequence may encompass 50-2000 adenines. |
| 22 | PD1 CAR (aa) | pgwfldspdrpwnpptfspallvvtegdnatftcsfsntsesfylnwyrmspsnqtdklaafpedrsqpgqdc |
| | (PD1 ECD | rfrvtqlpngrdfhmsvvrarrndsgtylcgaislapkaqikeslraelryterraevptahpspsprpagqfqtlv |
| | underlined) | tapaprpptpaptiasqplslrpeacrpaaggavhtrgldfacdiyiwaplagtcgvlllslvitlyckrgrkkllyi |
| | | fkqpfmrpvqttqeedgcscrfpeeeeggcelrvkfsrsadapaykqgqnqlynelnlgrreeydvldkrrgr |
| | | dpemggkprrknpqeglynelqkdkmaeayseigmkgeragkghdglyqglstatkdtydalhmqalppr |
| 23 | PD-1 CAR (na) | atggccctccctgtcactgccctgcttctccccctcgcactcctgctccacgccgctagaccacccggatggtttct |
| | (PD1 ECD | ggactctccggatcgcccgtggaatcccccaaccttctcaccggcactcttggttgtgactgagggcgataatgcg |
| | underlined) | accttcacgtgctcgttctccaacacctccgaatcattcgtgctgaactggtaccgcatgagcccgtcaaaccagac |
| | | cgacaagctcgccgcgtttccggaagatcggtcgcaaccgggacaggattgtcggttccgcgtgactcaactgc |
| | | cgaatggcagagacttccacatgagcgtggtccgcgctaggcgaaacgactccgggacctacctgtgcggagc |
| | | catctcgctggcgcctaaggcccaaatcaaagagagcttgagggccgaactgagagtgaccgagcgcagagct |
| | | gaggtgccaactgcacatccatccccatcgcctcggcctgcggggcagtttcagaccctggtcacgaccactccg |
| | | gcgccgcgcccaccgactccggccccaactatcgcgagccagcccctgtcgctgaggccggaagcatgccgc |
| | | cctgccgccggaggtgctgtgcatacccggggattggacttcgcatgcgacatctacatttgggctcctctcgccg |
| | | gaacttgtggcgtgctccttctgtccctggtcatcaccctgtactgcaagcggggtcggaaaaagcttctgtacatttt |
| | | caagcagcccttcatgaggcccgtgcaaaccacccaggaggaggacggttgctcctgccggttccccgaagag |
| | | gaagaaggaggttgcgagctgcgcgtgaagttctcccggagcgccgacgcccccgcctataagcagggccag |
| | | aaccagctgtacaacgaactgaacctgggacggcgggaagagtacgatgtgctggacaagcggcgcggccgg |
| | | gaccccgaaatgggcgggaagcctagaagaaagaaccctcaggaaggcctgtataacgagctgcagaaggac |
| | | aagatggccgaggcctactccgaaattgggatgaagggagagcggcggaggggaaaggggcacgacggcct |
| | | gtaccaaggactgtccaccgccaccaaggacacatacgatgccctgcacatgcaggcccttccccctcgc |
| 24 | PD-1 CAR (aa) | Malpvtalllplalllhaarppgwfldspdrpwnpptfspallvvtegdnatftcsfsntsesfylnwyrmspsn |
| | with signal | qtdklaafpedrsqpgqdcrfrvtqlpngrdfhmsvvrarrndsgtylcgaislapkaqikeslraelrvterrae |
| | (PD1 ECD | vptahpspsprpagqfqtlvtttpaprpptpaptiasqplslrpeacrpaaggavhtrgldfacdiyiwaplagtc |
| | underlined) | gvlllslvitlyckrgrkkllyifkqpfmrpvqttqeedgcscrfpeeeeggcelrvkfsrsadapaykqgqnql |
| | | ynelnlgrreeydvldkrrgrdpemggkprrknpqeglynelqkdkmaeayseigmkgerrrgkghdgly |
| | | qglstatkdtydalhmqalppr |

In one aspect, an exemplary CAR constructs comprise an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain decribed herein). In one aspect, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

In one aspect, the CARs (e.g., CD19 CARs) of the invention comprise at least one intracellular signaling domain selected from the group of a CD137 (4-1BB) signaling domain, a CD28 signaling domain, a CD27 signaling domain, an ICOS signaling domain, a CD3zeta signal domain, and any combination thereof. In one aspect, the CARs of the invention comprise at least one intracellular signaling domain is from one or more costimulatory molecule(s) selected from CD137 (4-1BB), CD28, CD27, or ICOS.

Exemplary CD19 CARs include CD19 CARs described herein, e.g., in one or more tables described herein, or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

### Antigen Binding Domain

In one aspect, the CAR of the disclosure comprises a target-specific binding element otherwise referred to as an antigen binding domain. In one embodiment, the portion of the CAR comprising the antigen binding domain comprises an antigen binding domain that targets, e.g., specifically binds to, an antigen, e.g., antigen described herein, e.g., CD19. In one embodiment, the antigen binding domain targets, e.g., specifically binds to, human CD19.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as an antigen binding domain, such as a recombinant fibronectin domain, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. Thus, in one aspect, the antigen binding domain comprises a human antibody or an antibody fragment.

In one embodiment, the antigen binding domain comprises one, two three (e.g., all three) heavy chain CDRs, HC CDR1, HC CDR2 and HC CDR3, from an antibody described herein (e.g., an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference), and/or one, two, three (e.g., all three) light chain CDRs, LC CDR1, LC CDR2 and LC CDR3, from an antibody described herein (e.g., an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference). In one embodiment, the antigen binding domain comprises a heavy chain variable region and/or a variable light chain region of an antibody listed above.

In embodiments, the antigen binding domain is an antigen binding domain described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference.

In embodiments, the antigen binding domain targets BCMA and is described in US-2016-0046724-A1.

In embodiments, the antigen binding domain targets CD19 and is described in US-2015-0283178-A1.

In embodiments, the antigen binding domain targets CD123 and is described in US2014/0322212A1, US2016/0068601A1.

In embodiments, the antigen binding domain targets CLL and is described in US2016/0051651A1.

In embodiments, the antigen binding domain targets CD33 and is described in US2016/0096892A1.

Exemplary target antigens that can be targeted using the CAR-expressing cells, include, but are not limited to, CD19, CD123, EGFRvIII, CD33, mesothelin, BCMA, and GFR ALPHA-4, among others, as described in, for example, WO2014/153270, WO 2014/130635, WO2016/028896, WO 2014/130657, WO2016/014576, WO 2015/090230, WO2016/014565, WO2016/014535, and WO2016/025880, each of which is herein incorporated by reference in its entirety.

In other embodiments, the CAR-expressing cells can specifically bind to humanized CD19, e.g., can include a CAR molecule, or an antigen binding domain (e.g., a humanized antigen binding domain) according to Table 3 of WO2014/153270, incorporated herein by reference. The amino acid and nucleotide sequences encoding the CD19 CAR molecules and antigen binding domains (e.g., including one, two, three VH CDRs; and one, two, three VL CDRs according to Kabat or Chothia), are specified in WO2014/153270.

In other embodiments, the CAR-expressing cells can specifically bind to CD123, e.g., can include a CAR molecule (e.g., any of the CAR1 to CAR8), or an antigen binding domain according to Tables 1-2 of WO 2014/130635, incorporated herein by reference. The amino acid and nucleotide sequences encoding the CD123 CAR molecules and antigen binding domains (e.g., including one, two, three VH CDRs; and one, two, three VL CDRs according to Kabat or Chothia), are specified in WO 2014/130635.

In other embodiments, the CAR-expressing cells can specifically bind to CD123, e.g., can include a CAR molecule (e.g., any of the CAR123-1 ro CAR123-4 and hzCAR123-1 to hzCAR123-32), or an antigen binding domain according to Tables 2, 6, and 9 of WO2016/028896, incorporated herein by reference. The amino acid and nucleotide sequences encoding the CD123 CAR molecules and antigen binding domains (e.g., including one, two, three VH CDRs; and one, two, three VL CDRs according to Kabat or Chothia), are specified in WO2016/028896.

In other embodiments, the CAR-expressing cells can specifically bind to EGFRvIII, e.g., can include a CAR molecule, or an antigen binding domain according to Table 2 or SEQ ID NO:11 of WO 2014/130657, incorporated herein by reference. The amino acid and nucleotide sequences encoding the EGFRvIII CAR molecules and antigen binding domains (e.g., including one, two, three VH CDRs; and one, two, three VL CDRs according to Kabat or Chothia), are specified in WO 2014/130657.

In other embodiments, the CAR-expressing cells can specifically bind to CD33, e.g., can include a CAR molecule (e.g., any of CAR33-1 to CAR-33-9), or an antigen binding domain according to Table 2 or 9 of WO2016/014576, incorporated herein by reference. The amino acid and nucleotide sequences encoding the CD33 CAR molecules and antigen binding domains (e.g., including one, two, three VH CDRs; and one, two, three VL CDRs according to Kabat or Chothia), are specified in WO2016/014576.

In other embodiments, the CAR-expressing cells can specifically bind to mesothelin, e.g., can include a CAR molecule, or an antigen binding domain according to Tables 2-3 of WO 2015/090230, incorporated herein by reference. The amino acid and nucleotide sequences encoding the mesothelin CAR molecules and antigen binding domains (e.g., including one, two, three VH CDRs; and one, two, three VL CDRs according to Kabat or Chothia), are specified in WO 2015/090230.

In other embodiments, the CAR-expressing cells can specifically bind to BCMA, e.g., can include a CAR molecule, or an antigen binding domain according to Table 1 or 16, SEQ ID NO: 271 or SEQ ID NO: 273 of WO2016/014565, incorporated herein by reference. The amino acid and nucleotide sequences encoding the BCMA CAR molecules and antigen binding domains (e.g., including one, two, three VH CDRs; and one, two, three VL CDRs according to Kabat or Chothia), are specified in WO2016/014565.

In other embodiments, the CAR-expressing cells can specifically bind to CLL-1, e.g., can include a CAR molecule, or an antigen binding domain according to Table 2 of WO2016/014535, incorporated herein by reference. The amino acid and nucleotide sequences encoding the CLL-1 CAR molecules and antigen binding domains (e.g., including one, two, three VH CDRs; and one, two, three VL CDRs according to Kabat or Chothia), are specified in WO2016/014535.

In other embodiments, the CAR-expressing cells can specifically bind to GFR ALPHA-4, e.g., can include a CAR molecule, or an antigen binding domain according to Table 2 of WO2016/025880, incorporated herein by reference. The amino acid and nucleotide sequences encoding the GFR ALPHA-4 CAR molecules and antigen binding domains (e.g., including one, two, three VH CDRs; and one, two, three VL CDRs according to Kabat or Chothia), are specified in WO2016/025880.

In one embodiment, the antigen binding domain of any of the CAR molecules described herein (e.g., any of CD19, CD123, EGFRvIII, CD33, mesothelin, BCMA, and GFR ALPHA-4) comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antigen binding domain listed above. In one embodiment, the antigen binding domain comprises a heavy chain variable region and/or a variable light chain region of an antibody listed or described above.

In one embodiment, the CD19 binding domain comprises one or more (e.g., all three) light chain complementary determining region 1 (LC CDR1), light chain complementary determining region 2 (LC CDR2), and light chain complementary determining region 3 (LC CDR3) of a CD19 binding domain selected from SEQ ID NOS: 45-56, 69-80, 106, 109, 110, 112, or 115 and one or more (e.g., all three) heavy chain complementary determining region 1 (HC CDR1), heavy chain complementary determining region 2 (HC CDR2), and heavy chain complementary determining region 3 (HC CDR3) of a CD19 binding domain selected from SEQ ID NOS: 45-56, 69-80, 106, 109, 110, 112, or 115. In one embodiment, the CD19 binding domain comprises a light chain variable region described herein (e.g., in Table 2 or 3) and/or a heavy chain variable region described herein (e.g., in Table 2 or 3). In one embodiment, the CD19 binding domain is a scFv comprising a light chain variable region and a heavy chain variable region of an amino acid sequence of Table 2 or 3. In an embodiment, the CD19 binding domain (e.g., an scFV) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 2 or 3, or a sequence with 95-99% identity to an amino acid sequence of Table 2 or 3; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 2 or 3, or a sequence with 95-99% identity to an amino acid sequence of Table 2 or 3.

In one embodiment, the CD19 binding domain comprises a light chain variable region comprising an amino acid sequence described herein, e.g., in Table 2 or 3, is attached to a heavy chain variable region comprising an amino acid sequence described herein, e.g., in Table 2 or 3, via a linker, e.g., a linker described herein. In one embodiment, the humanized anti-CD19 binding domain includes a (Gly4-Ser)n linker (SEQ ID NO: 26), wherein n is 1, 2, 3, 4, 5, or 6, preferably 3 or 4. The light chain variable region and heavy chain variable region of a scFv can be, e.g., in any of the following orientations: light chain variable region-linker-heavy chain variable region or heavy chain variable region-linker-light chain variable region.

In another embodiment, the CD19 binding domain comprises any antibody or antibody fragment thereof known in the art that binds to CD19.

In one aspect, the antibodies of the invention may exist in a variety of other forms including, for example, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide brudge at the hinge region, and an isolated CDR or other epitope binding fragments of an antibody. In one aspect, the antibody fragment provided herein is a scFv. In some instances, a human scFv may also be derived from a yeast display library.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. No. 6,407,213, U.S. Pat. No. 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Additional information on framework regions and humanized antibodies is described on pages 169-170 of International Application WO 2016/164731, filed Apr. 8, 2016, which is incorporated by reference in its entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a VH4_4-59 germline sequence. In one embodiment, the framework region can comprise, one, two, three, four or five modifications, e.g., substitutions, e.g., from the amino acid at the corresponding murine sequence (e.g., of SEQ ID NO: 109). In one embodiment, the framework region, e.g., all four framework regions of the light chain variable region are derived from a VK3_1.25 germline sequence. In one embodiment, the framework region can comprise, one, two, three, four or five modifications, e.g., substitutions, e.g., from the amino acid at the corresponding murine sequence (e.g., of SEQ ID NO: 109). Design of humanized antibodies or antibody fragments based on three-dimensional conformational structure is described in detail on page 171 of International Application WO 2016/164731, filed Apr. 8, 2016, which is incorporated by reference in its entirety.

A humanized antibody or antibody fragment may retain a similar antigenic specificity as the original antibody, e.g., in the present disclosure, the ability to bind human CD19. In some embodiments, a humanized antibody or antibody fragment may have improved affinity and/or specificity of binding to human CD19.

In one aspect, the binding domain (e.g., an antigen-binding domain that binds CD19) is a fragment, e.g., a single chain variable fragment (scFv). In one aspect, the binding domain is a Fv, a Fab, a (Fab′)2, or a bi-functional (e.g. bi-specific) hybrid antibody (e.g., Lanzavecchia et al., Eur. J. Immunol. 17, 105 (1987)). In one aspect, the antibodies and fragments thereof of the invention binds a CD19 protein with wild-type or enhanced affinity.

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO:25). In one embodiment, the linker can be (Gly₄Ser)₄ (SEQ ID NO:27) or (Gly₄Ser)₃(SEQ ID NO:28). Variation in the linker length may retain or enhance activity, giving rise to superior efficacy in activity studies.

In some embodiments, the amino acid sequence of the antigen binding domain (e.g., an antigen-binding domain that binds CD19) or other portions or the entire CAR can be modified, e.g., an amino acid sequence described herein can be modified, e.g., by a conservative substitution. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%. 72%. 73%, 74%, 75%, 76%, 77%, 78%, 79%, 80%,81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

In one aspect, the present disclosure contemplates modifications of the starting antibody or fragment (e.g., scFv) amino acid sequence that generate functionally equivalent molecules. For example, the VH or VL of a binding domain (e.g., an antigen-binding domain that binds CD19), e.g., scFv, comprised in the CAR can be modified to retain at least about 70%, 71%. 72%. 73%, 74%, 75%, 76%, 77%, 78%, 79%, 80%,81%, 82%, 83%, 84%, 85%, 86%, 87%, 88%, 89%, 90%, 91%, 92%, 93%, 94%, 95%, 96%, 97%, 98%, 99% identity of the starting VH or VL framework region of an anti-CD19 binding domain, e.g., scFv. The present invention contemplates modifications of the entire CAR construct, e.g., modifications in one or more amino acid sequences of the various domains of the CAR construct in order to generate functionally equivalent molecules. The CAR construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting CAR construct.

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together, e.g., using flexible polypeptide linkers. The scFv molecules can comprise a linker (e.g., a Ser-Gly linker) with an optimized length and/or amino acid composition. The linker length can greatly affect how the variable regions of an scFv fold and interact. In fact, if a short polypeptide linker is employed (e.g., between 5-10 amino acids, intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

### Exemplary CD19 Antigen Binding Domains and CAR Constructs

Exemplary CD19 CAR constructs disclosed herein comprise a scFv (e.g., a human scFv) as disclosed in Table 2 or 3 herein, optionally preceded with an optional leader sequence (e.g., SEQ ID NO:1 and SEQ ID NO:12 for exemplary leader amino acid and nucleotide sequences, respectively). The sequences of the scFv fragments (amino acid sequences of SEQ ID NOs: 45-56, 69-80, 106, 109, 110, 112, or 115) are provided herein in Table 2 or 3. The CD19 CAR construct can further include an optional hinge domain, e.g., a CD8 hinge domain (e.g., including the amino acid sequence of SEQ ID NO: 2 or encoded by a nucleic acid sequence of SEQ ID NO:13); a transmembrane domain, e.g., a CD8 transmembrane domain (e.g., including the amino acid sequence of SEQ ID NO: 6 or encoded by the nucleotide sequence of SEQ ID NO: 17); an intracellular domain, e.g., a 4-1BB intracellular domain (e.g., including the amino acid sequence of SEQ ID NO: 7 or encoded by the nucleotide sequence of SEQ ID NO: 18; and a functional signaling domain, e.g., a CD3 zeta domain (e.g., including amino acid sequence of SEQ ID NO: 9 or 10, or encoded by the nucleotide sequence of SEQ ID NO: 20 or 21). In certain embodiments, the domains are contiguous with and in the same reading frame to form a single fusion protein. In other embodiments, the domain are in separate polypeptides, e.g., as in an RCAR molecule as described herein.

In certain embodiments, the full length CD19 CAR molecule includes the amino acid sequence of, or is encoded by the nucleotide sequence of, CAR1-CAR12, CTL019, mCAR1-mCAR3, or SSJ25-C1, provided in Table 2 or 3, or a sequence substantially identical (e.g., 95-99% identical thereto, or up to 20, 15, 10, 8, 6, 5, 4, 3, 2, or 1 amino acid changes) to any of the aforesaid sequences.

In certain embodiments, the CD19 CAR molecule, or the CD19 antigen binding domain, includes the scFv amino acid sequence of, or is encoded by the nucleotide sequence of, CAR1-CAR12, CTL019, mCAR1-mCAR3, or SSJ25-C1 , provided in Table 2 or 3, or a sequence substantially identical (e.g., 95-99% identical thereto, or up to 20, 15, 10, 8, 6, 5, 4, 3, 2, or 1 amino acid changes) to any of the aforesaid sequences.

In certain embodiments, the CD19 CAR molecule, or the CD19 antigen binding domain, includes the heavy chain variable region and/or the light chain variable region of CAR1-CAR12, CTL019, mCAR1-mCAR3, or SSJ25-C1 , provided in Table 2 or 3, or a sequence substantially identical (e.g., 95-99% identical, or up to 20, 15, 10, 8, 6, 5, 4, 3, 2, or 1 amino acid changes) to any of the aforesaid sequences.

In certain embodiments, the CD19 CAR molecule, or the CD19 antigen binding domain, includes one, two or three CDRs from the heavy chain variable region (e.g., HCDR1, HCDR2 and/or HCDR3) of CAR1-CAR12, CTL019, mCAR1-mCAR3, or SSJ25-C1,provided in Table 2 or 3; and/or one, two or three CDRs from the light chain variable region (e.g., LCDR1, LCDR2 and/or LCDR3) of CAR1-CAR12, CTL019, mCAR1-mCAR3, or SSJ25-C1, provided in Table 2 or 3; or a sequence substantially identical (e.g., 95-99% identical, or up to 5, 4, 3, 2, or 1 amino acid changes) to any of the aforesaid sequences.

The sequences of CDR sequences of the scFv domains are shown in Table 4 for the heavy chain variable domains and in Table 5 for the light chain variable domains.

The amino acid and nucleic acid sequences of the CD19 scFv domains and CD19 CAR molecules are provided in Tables 2 and 3. In one embodiment, the CD19 CAR molecule includes a leader sequence described herein, e.g., as underlined in the sequences provided in Tables 2 and 3. In one embodiment, the CD19 CAR molecule does not include a leader sequence.

In embodiments, the CAR molecule comprises an antigen binding domain that binds specifically to CD19 (CD19 CAR). In one embodiment, the antigen binding domain targets human CD19. In one embodiment, the antigen binding domain of the CAR has the same or a similar binding specificity as the FMC63 scFv fragment described in Nicholson et al. Mol. Immun. 34 (16-17): 1157-1165 (1997). In one embodiment, the antigen binding domain of the CAR includes the scFv fragment described in Nicholson et al. Mol. Immun. 34 (16-17): 1157-1165 (1997). A CD19 antibody molecule can be, e.g., an antibody molecule (e.g., a humanized anti-CD19 antibody molecule) described in WO2014/153270, which is incorporated herein by reference in its entirety. WO2014/153270 also describes methods of assaying the binding and efficacy of various CAR constructs.

In one aspect, the parental murine scFv sequence is the CAR19 construct provided in PCT publication WO2012/079000 (incorporated herein by reference) and provided herein as SEQ ID NO: 108. In one embodiment, the anti-CD19 binding domain is a scFv described in WO2012/079000 and provided herein in SEQ ID NO: 109.

In one embodiment, the CAR molecule comprises the polypeptide sequence provided as SEQ ID NO: 12 in PCT publication WO2012/079000, and provided herein as SEQ ID NO: 108, wherein the scFv domain is substituted by one or more sequences selected from SEQ ID NOS: 93-104. In one embodiment, the scFv domains of SEQ ID NOS: 93-104 are humanized variants of the scFv domain of SEQ ID NO: 109 which is an scFv fragment of murine origin that specifically binds to human CD19. Humanization of this mouse scFv may be desired for the clinical setting, where the mouse-specific residues may induce a human-anti-mouse antigen (HAMA) response in patients who receive CART19 treatment, e.g., treatment with T cells transduced with the CAR19 construct.

In one embodiment, the CD19 CAR comprises an amino acid sequence provided as SEQ ID NO: 12 in PCT publication WO2012/079000. In embodiment, the amino acid sequence is

MALPVTALLLPLALLLHAARPdiqmtqttsslsaslgdrvtiscrasqdiskylnwyqqkpdgtvklliyhtsr lhsgvpsrfsgsgsgtdysltisnleqediatyfcqqgntlpytfgggtkleitggggsggggsggggsevklqesgpglvapsqslsvtct vsgvslpdygvswirqpprkglewlgviwgsettyynsalksrltiikdnsksqvflkmnslqtddtaiyycakhyyyggsyamdyw gqgtsvtvsstttpaprpptpaptiasqplslrpeacrpaaggavhtrgldfacdiyiwaplagtcgvlllslvitlyckrgrkkllyifkqpf mrpvqttqeedgcscrfpeeeeggcelrvkfsrsadapaykqgqnqlynelnlgrreeydvldkrrgrdpemggkprrknpqeglyn elqkdkmaeayseigmkgerrrgkghdglyqglstatkdtydalhmqalppr (SEQ ID NO: 108), or a sequence substantially homologous thereto.

In one embodiment, the amino acid sequence is:

diqmtqttsslsaslgdrvtiscrasqdiskylnwyqqkpdgtvklliyhtsrlhsgvpsrfsgsgsgtdysltisnleqediatyf cqqgntlpytfgggtkleitggggsggggsggggsevklqesgpglvapsqslsvtctvsgvslpdygvswirqpprkglewlgviwg settyynsalksrltiikdnsksqvflkmnslqtddtaiyycakhyyyggsyamdywgqgtsvtvsstttpaprpptpaptiasqplslrp eacrpaaggavhtrgldfacdiyiwaplagtcgvlllslvitlyckrgrkkllyifkqpfmrpvqttqeedgcscrfpeeeeggcelrvkfs rsadapaykqgqnqlynelnlgrreeydvldkrrgrdpemggkprrknpqeglynelqkdkmaeayseigmkgerrrgkghdgly qglstatkdtydalhmqalppr (SEQ ID NO: 289), or a sequence substantially homologous thereto.

In one embodiment, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In other embodiments, the CD19 CAR comprises an antigen binding domain (e.g., a humanized antigen binding domain) according to Table 3 of WO2014/153270, incorporated herein by reference.

In embodiments, the CAR molecule is a CD19 CAR molecule described herein, e.g., a humanized CAR molecule described herein, e.g., a humanized CD19 CAR molecule of Table 2 or having CDRs as set out in Tables 4 and 5.

In embodiments, the CAR molecule is a CD19 CAR molecule described herein, e.g., a murine CAR molecule described herein, e.g., a murine CD19 CAR molecule of Table 3 or having CDRs as set out in Tables 4 and 5.

In some embodiments, the CAR molecule comprises one, two, and/or three CDRs from the heavy chain variable region and/or one, two, and/or three CDRs from the light chain variable region of the murine or humanized CD19 CAR of Table 4 and 5.

In one embodiment, the antigen binding domain comprises one, two three (e.g., all three) heavy chain CDRs, HC CDR1, HC CDR2 and HC CDR3, from an antibody listed herein, and/or one, two, three (e.g., all three) light chain CDRs, LC CDR1, LC CDR2 and LC CDR3, from an antibody listed herein. In one embodiment, the antigen binding domain comprises a heavy chain variable region and/or a variable light chain region of an antibody listed herein.

### Humanization of Murine Anti-CD19 Antibody

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159), for instance Tables 3, 4, and 5 (p. 125-147).

### CAR Constructs, e.g., CD19 CAR Constructs

Of the CD19 CAR constructs described in International Application WO2014/153270, certain sequences are reproduced herein.

The sequences of the humanized scFv fragments (SEQ ID NOS: 45-56) are provided below in Table 2. Full CAR constructs were generated using SEQ ID NOs: 45-56 with additional sequences, e.g., from Table 1, shown below, to generate full CAR constructs with SEQ ID NOs: 93-104.

These clones all contained a Q/K residue change in the signal domain of the co-stimulatory domain derived from 4-1BB.

TABLE 2
Humanized CD19 CAR Constructs

| | | |
|---|---|---|
| | SEQ | |
| Name | ID NO: | Sequence |

CAR 1

| | | |
|---|---|---|
| CAR1 | 45 | EIVMTQSPATLSLSPGERATLSCRASQDISKYLNWYQQKPGQAPRLL |
| scFv | | IYHTSRLHSGIPARFSGSGSGTDYTLTISSLQPEDFAVYFCQQGNTL |
| domain | | PYTFGQGTKLEIKGGGGSGGGGSGGGGSQVQLQESGPGLVKPSETLS |
| | | LTCTVSGVSLPDYGVSWIRQPPGKGLEWIGVIWGSETTYYSSSLKSR |
| | | VIISKDNSKNQVSLKLSSVTAADTAVYYCAKHYYYGGSYAMDYWGQG |
| | | TLVTVSS |
| 103101 | 57 | atggccctccctgtcaccgccctgctgcttccgctggctcttctgctccacgccgc |
| CAR1 | | tcggcccgaaattgtgatgacccagtcacccgccactcttagcctttcacccggtg |
| Soluble | | agcgcgcaaccctgtcttgcagagcctcccaagacatctcaaaataccttaattgg |
| scFv-nt | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactactcttcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagccaccaccatcatcaccatcaccat |
| 103101 | 69 | MALPVTALLLPLALLLHAARPeivmtqspatlslspgeratlscrasqdiskylnw |
| CAR1 | | yqqkpgqaprlliyhtsrlhsgiparfsgsgsgtdytltisslqpedfavyfcqqg |
| Soluble | | ntlpytfgqgtkleikggggsggggsggggsqvqlqesgpglvkpsetlsltctvs |
| scFv-aa | | gvslpdygvswirqppgkglewigviwgsettyyssslksrvtiskdnsknqvslk |
| | | lssvtaadtavyycakhyyyggsyamdywgqgtlvtvsshhhhhhhh |
| 104875 | 81 | atggccctccctgtcaccgccctgctgcttccgctggctcttctgctccacgccgc |
| CAR 1- | | tcggcccgaaattgtgatgacccagtcacccgccactcttagcctttcacccggtg |
| Full-nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactactcttcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagcaccactaccccagcaccgaggccacccaccccggctcctaccatcgcctcc |
| | | cagcctctgtccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 104875 | 93 | MALPVTALLLPLALLLHAARPeivmtqspatlslspgeratlscrasqdiskylnw |
| CAR 1- | | yqqkpgqaprlliyhtsrlhsgiparfsgsgsgtdytltisslqpedfavyfcqqg |
| Full-aa | | ntlpytfgqgtkleikggggsggggsggggsqvqlqesgpglvkpsetlsltctvs |
| | | gvslpdygvswirqppgkglewigviwgsettyyssslksrvtiskdnsknqvslk |
| | | lssvtaadtavyycakhyyyggsyamdywgqgtlvtvsstttpaprpptpaptias |
| | | qplslrpeacrpaaggavhtrgldfacdiyiwaplagtcgvlllslvitlyckrgr |
| | | kkllyifkqpfmrpvqttqeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |

CAR2

| | | |
|---|---|---|
| CAR2 | 46 | eivmtqspatlslspgeratlscrasqdiskylnwyqqkpgqaprlliyhtsrlhs |
| scFv | | giparfsgsgsgtdytltisslqpedfavyfcqqgntlpytfgqgtkleikggggs |
| domain | | ggggsggggsqvqlqesgpglvkpsetlsltctvsgvslpdygvswirqppgkgle |
| | | wigviwgsettyyqsslksrvtiskdnsknqvslklssvtaadtavyycakhyyyg |
| | | gsyamdywgqgtlvtvss |
| 103102 | 58 | atggccctccctgtcaccgccctgctgcttccgctggctcttctgctccacgccgc |
| CAR2- | | tcggcccgaaattgtgatgacccagtcacccgccactcttagcctttcacccggtg |
| Soluble | | agcgcgcaaccctgtcttgcagagcctcccaagacatctcaaaataccttaattgg |
| scFv-nt | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactaccaatcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagccaccaccatcatcaccatcaccat |
| 103102 | 70 | MALPVTALLLPLALLLHAARPeivmtqspatlslspgeratlscrasqdiskylnw |
| CAR2- | | yqqkpgqaprlliyhtsrlhsgiparfsgsgsgtdytltisslqpedfavyfcqqg |
| Soluble | | ntlpytfgqgtkleikggggsggggsggggsqvqlqesgpglvkpsetlsltctvs |
| scFv-aa | | gvslpdygvswirqppgkglewigviwgsettyyqsslksrvtiskdnsknqvslk |
| | | lssvtaadtavyycakhyyyggsyamdywgqgtlvtvsshhhhhhhh |
| 104876 | 82 | atggccctccctgtcaccgccctgctgcttccgctggctcttctgctccacgccgc |
| CAR 2- | | tcggcccgaaattgtgatgacccagtcacccgccactcttagcctttcacccggtg |
| Full-nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactaccaatcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagcaccactaccccagcaccgaggccacccaccccggctcctaccatcgcctcc |
| | | cagcctctgtccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 104876 | 94 | MALPVTALLLPLALLLHAARPeivmtqspatlslspgeratlscrasqdiskylnw |
| CAR 2- | | yqqkpgqaprlliyhtsrlhsgiparfsgsgsgtdytltisslqpedfavyfcqqg |
| Full-aa | | ntlpytfgqgtkleikggggsggggsggggsqvqlqesgpglvkpsetlsltctvs |
| | | gvslpdygvswirqppgkglewigviwgsettyyqsslksrvtiskdnsknqvslk |
| | | lssvtaadtavyycakhyyyggsyamdywgqgtivtvsstttpaprpptpaptias |
| | | qplslrpeacrpaaggavhtrgldfacdiyiwaplagtcgvlllslvitlyckrgr |
| | | kkllyifkqpfmrpvqttgeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |

CAR 3

| | | |
|---|---|---|
| CAR3 | 47 | qvqlqesgpglvkpsetlsltctvsgvslpdygvswirqppgkglewigviwgset |
| scFv | | tyyssslksrvtiskdnsknqvslklssvtaadtavyycakhyyyggsyamdywgq |
| domain | | gtlvtvssggggsggggsggggseivmtqspatlslspgeratlscrasqdiskyl |
| | | nwyqqkpgqaprlliyhtsrlhsgiparfsgsgsgtdytltisslqpedfavyfcq |
| | | qgntlpytfgqgtkleik |
| 103104 | 59 | atggctctgcccgtgaccgcactcctcctgccactggctctgctgcttcacgccgc |
| CAR 3- | | tcgcccacaagtccagcttcaagaatcagggcctggtctggtgaagccatctgaga |
| Soluble | | ctctgtccctcacttgcaccgtgagcggagtgtccctcccagactacggagtgagc |
| scFv-nt | | tggattagacagcctcccggaaagggactggagtggatcggagtgatttggggtag |
| | | cgaaaccacttactattcatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaacatcaccaccatcatcaccatcac |
| 103104 | 71 | MALPVTALLLPLALLLHAARPqvqlqesgpglvkpsetlsltctvsgvslpdygvs |
| CAR 3- | | wirqppgkglewigviwgsettyyssslksrvtiskdnsknqvslklssvtaadta |
| Soluble | | vyycakhyyyggsyamdywgqgtlvtvssggggsggggsggggseivmtqspatls |
| scFv-aa | | lspgeratlscrasqdiskylnwyqqkpgqaprlliyhtsrlhsgiparfsgsgsg |
| | | tdytltisslqpedfavyfcqqgntlpytfgqgtkleikhhhhhhhh |
| 104877 | 83 | atggctctgcccgtgaccgcactcctcctgccactggctctgctgcttcacgccgc |
| CAR 3- | | tcgcccacaagtccagcttcaagaatcagggcctggtctggtgaagccatctgaga |
| Full-nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactattcatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaaaccactactcccgctccaaggccacccacccctgccccgaccatcgcctct |
| | | cagccgctttccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 104877 | 95 | MALPVTALLLPLALLLHAARPqvqlqesgpglvkpsetlsltctvsgvslpdygvs |
| CAR 3- | | wirqppgkglewigviwgsettyyssslksrvtiskdnsknqvslklssvtaadta |
| Full-aa | | vyycakhyyyggsyamdywgqgtivtvssggggsggggsggggseivmtqspatls |
| | | lspgeratlscrasqdiskylnwyqqkpgqaprlliyhtsrlhsgiparfsgsgsg |
| | | tdytltisslqpedfavyfcqqgntlpytfgqgtkleiktttpaprpptpaptias |
| | | qplslrpeacrpaaggavhtrgldfacdiyiwaplagtcgvlllslvitlyckrgr |
| | | kkllyifkqpfmrpvqttqeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |

CAR 4

| | | |
|---|---|---|
| CAR4 | 48 | qvqlqesgpglvkpsetlsltctvsgvslpdygvswirqppgkglewigviwgset |
| scBT | | tyyqsslksrvtiskdnsknqvslklssvtaadtavyycakhyyyggsyamdywgq |
| domain | | gtlvtvssggggsggggsggggseivmtqspatlslspgeratlscrasqdiskyl |
| | | nwyqqkpgqaprlliyhtsrlhsgiparfsgsgsgtdytltisslqpedfavyfcq |
| | | qgntlpytfgqgtkleik |
| 103106 | 60 | atggctctgcccgtgaccgcactcctcctgccactggctctgctgcttcacgccgc |
| CAR4- | | tcgcccacaagtccagcttcaagaatcagggcctggtctggtgaagccatctgaga |
| Soluble | | ctctgtccctcacttgcaccgtgagcggagtgtccctcccagactacggagtgagc |
| scFv-nt | | tggattagacagcctcccggaaagggactggagtggatcggagtgatttggggtag |
| | | cgaaaccacttactatcaatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaacatcaccaccatcatcaccatcac |
| 103106 | 72 | MALPVTALLLPLALLLHAARPqvqlqesgpglvkpsetlsltctvsgvslpdygvs |
| CAR4- | | wirqppgkglewigviwgsettyyqsslksrvtiskdnsknqvslklssvtaadta |
| Soluble | | vyycakhyyyggsyamdywgqgtlvtvssggggsggggsggggseivmtqspatls |
| scFv-aa | | lspgeratlscrasqdiskylnwyqqkpgqaprlliyhtsrlhsgiparfsgsgsg |
| | | tdytltisslqpedfavyfcqqgntlpytfgqgtkleikhhhhhhhh |
| 104878 | 84 | atggctctgcccgtgaccgcactcctcctgccactggctctgctgcttcacgccgc |
| CAR 4- | | tcgcccacaagtccagcttcaagaatcagggcctggtctggtgaagccatctgaga |
| Full-nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactatcaatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaaaccactactcccgctccaaggccacccacccctgccccgaccatcgcctct |
| | | cagccgctttccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 104878 | 96 | MALPVTALLLPLALLLHAARPqvqlqesgpglvkpsetlsltctvsgvslpdygvs |
| CAR 4- | | wirqppgkglewigviwgsettyyqsslksrvtiskdnsknqvslklssvtaadta |
| Full-aa | | vyycakhyyyggsyamdywgqgtivtvssggggsggggsggggseivmtqspatls |
| | | lspgeratlscrasgdiskylnwyqqkpgqaprlliyhtsrlhsgiparfsgsgsg |
| | | tdytltisslqpedfavyfcqqgntlpytfgqgtkleiktttpaprpptpaptias |
| | | qplslrpeacrpaaggavhtrgldfacdiyiwaplagtcgvlllslvitlyckrgr |
| | | kkllyifkqpfmrpvqttgeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |

CAR5

| | | |
|---|---|---|
| CAR5 | 49 | eivmtqspatlslspgeratlscrasqdiskylnwyqqkpgqaprlliyhtsrlhs |
| scBT | | giparfsgsgsgtdytltisslqpedfavyfcqqgntlpytfgqgtkleikggggs |
| domain | | ggggsggggsggggsqvqlqesgpglvkpsetlsltctvsgvslpdygvswirqpp |
| | | gkglewigviwgsettyyssslksrvtiskdnsknqvslklssvtaadtavyycak |
| | | hyyyggsyamdywgqgtlvtvss |
| 99789 | 61 | atggccctcccagtgaccgctctgctgctgcctctcgcacttcttctccatgccgc |
| CAR5- | | tcggcctgagatcgtcatgacccaaagccccgctaccctgtccctgtcacccggcg |
| Soluble | | agagggcaaccctttcatgcagggccagccaggacatttctaagtacctcaactgg |
| scFv-nt | | tatcagcagaagccagggcaggctcctcgcctgctgatctaccacaccagccgcct |
| | | ccacagcggtatccccgccagattttccgggagcgggtctggaaccgactacaccc |
| | | tcaccatctcttctctgcagcccgaggatttcgccgtctatttctgccagcagggg |
| | | aatactctgccgtacaccttcggtcaaggtaccaagctggaaatcaagggaggcgg |
| | | aggatcaggcggtggcggaagcggaggaggtggctccggaggaggaggttcccaag |
| | | tgcagcttcaagaatcaggacccggacttgtgaagccatcagaaaccctctccctg |
| | | acttgtaccgtgtccggtgtgagcctccccgactacggagtctcttggattcgcca |
| | | gcctccggggaagggtcttgaatggattggggtgatttggggatcagagactactt |
| | | actactcttcatcacttaagtcacgggtcaccatcagcaaagataatagcaagaac |
| | | caagtgtcacttaagctgtcatctgtgaccgccgctgacaccgccgtgtactattg |
| | | tgccaaacattactattacggagggtcttatgctatggactactggggacagggga |
| | | ccctggtgactgtctctagccatcaccatcaccaccatcatcac |
| 99789 | 73 | MALPVTALLLPLALLLHAARPeivmtqspatlslspgeratlscrasqdiskylnw |
| CAR5- | | yqqkpgqaprlliyhtsrlhsgiparfsgsgsgtdytltisslqpedfavyfcqqg |
| Soluble | | ntlpytfgqgtkleikggggsggggsggggsggggsqvqlqesgpglvkpsetlsl |
| scFv-aa | | tctvsgvslpdygvswirqppgkglewigviwgsettyyssslksrvtiskdnskn |
| | | qvslklssvtaadtavyycakhyyyggsyamdywgqgtivtvsshhhhhhhh |
| 104879 | 85 | atggccctccctgtcaccgccctgctgcttccgctggctcttctgctccacgccgc |
| CAR 5- | | tcggcccgaaattgtgatgacccagtcacccgccactcttagcctttcacccggtg |
| | | agcgcgcaaccctgtcttgcagagcctcccaagacatctcaaaataccttaattgg |
| Full-nt | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagcggcggaggcgggagccagg |
| | | tccaactccaagaaagcggaccgggtcttgtgaagccatcagaaactctttcactg |
| | | acttgtactgtgagcggagtgtctctccccgattacggggtgtcttggatcagaca |
| | | gccaccggggaagggtctggaatggattggagtgatttggggctctgagactactt |
| | | actactcttcatccctcaagtcacgcgtcaccatctcaaaggacaactctaagaat |
| | | caggtgtcactgaaactgtcatctgtgaccgcagccgacaccgccgtgtactattg |
| | | cgctaagcattactattatggcgggagctacgcaatggattactggggacagggta |
| | | ctctggtcaccgtgtccagcaccactaccccagcaccgaggccacccaccccggct |
| | | cctaccatcgcctcccagcctctgtccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 104879 | 97 | MALPVTALLLPLALLLHAARPeivmtqspatlslspgeratlscrasqdiskylnw |
| CAR 5- | | yqqkpgqaprlliyhtsrlhsgiparfsgsgsgtdytltisslqpedfavyfcqqg |
| Full-aa | | ntlpytfgqgtkleikggggsggggsggggsggggsqvqlqesgpglvkpsetlsl |
| | | tctvsgvslpdygvswirqppgkglewigviwgsettyyssslksrvtiskdnskn |
| | | qvslklssvtaadtavyycakhyyyggsyamdywgqgtivtvsstttpaprpptpa |
| | | ptiasqplslrpeacrpaaggavhtrgldfacdiyiwaplagtcgvlllslvitly |
| | | ckrgrkkllyifkqpfmrpvqttgeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR 6

| | | |
|---|---|---|
| CAR6 | 50 | eivmtqspatlslspgeratlscrasqdiskylnwyqqkpgqaprlliyhtsrlhs |
| scFv | | giparfsgsgsgtdytltisslqpedfavyfcqqgntlpytfgqgtkleikggggs |
| domain | | ggggsggggsggggsqvqlqesgpglvkpsetlsltctvsgvslpdygvswirqpp |
| | | gkglewigviwgsettyyqsslksrvtiskdnsknqvslklssvtaadtavyycak |
| | | hyyyggsyamdywgqgtlvtvss |
| 99790 | 62 | atggccctcccagtgaccgctctgctgctgcctctcgcacttcttctccatgccgc |
| CAR6- | | tcggcctgagatcgtcatgacccaaagccccgctaccctgtccctgtcacccggcg |
| Soluble | | agagggcaaccctttcatgcagggccagccaggacatttctaagtacctcaactgg |
| scFv-nt | | tatcagcagaagccagggcaggctcctcgcctgctgatctaccacaccagccgcct |
| | | ccacagcggtatccccgccagattttccgggagcgggtctggaaccgactacaccc |
| | | tcaccatctcttctctgcagcccgaggatttcgccgtctatttctgccagcagggg |
| | | aatactctgccgtacaccttcggtcaaggtaccaagctggaaatcaagggaggcgg |
| | | aggatcaggcggtggcggaagcggaggaggtggctccggaggaggaggttcccaag |
| | | tgcagcttcaagaatcaggacccggacttgtgaagccatcagaaaccctctccctg |
| | | acttgtaccgtgtccggtgtgagcctccccgactacggagtctcttggattcgcca |
| | | gcctccggggaagggtcttgaatggattggggtgatttggggatcagagactactt |
| | | actaccagtcatcacttaagtcacgggtcaccatcagcaaagataatagcaagaac |
| | | caagtgtcacttaagctgtcatctgtgaccgccgctgacaccgccgtgtactattg |
| | | tgccaaacattactattacggagggtcttatgctatggactactggggacagggga |
| | | ccctggtgactgtctctagccatcaccatcaccaccatcatcac |
| 99790 | 74 | MALPVTALLLPLALLLHAARPeivmtqspatlslspgeratlscrasqdiskylnw |
| CAR6- | | yqqkpgqaprlliyhtsrlhsgiparfsgsgsgtdytltisslqpedfavyfcqqg |
| Soluble | | ntlpytfgqgtkleikggggsggggsggggsggggsqvqlqesgpglvkpsetlsl |
| scFv-aa | | tctvsgvslpdygvswirqppgkglewigviwgsettyyqsslksrvtiskdnskn |
| | | qvslklssvtaadtavyycakhyyyggsyamdywgqgtivtvsshhhhhhhh |
| 104880 | 86 | atggccctccctgtcaccgccctgctgcttccgctggctcttctgctccacgccgc |
| CAR6- | | tcggcccgaaattgtgatgacccagtcacccgccactcttagcctttcacccggtg |
| Full-nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagcggaggcggagggagccagg |
| | | tccaactccaagaaagcggaccgggtcttgtgaagccatcagaaactctttcactg |
| | | acttgtactgtgagcggagtgtctctccccgattacggggtgtcttggatcagaca |
| | | gccaccggggaagggtctggaatggattggagtgatttggggctctgagactactt |
| | | actaccaatcatccctcaagtcacgcgtcaccatctcaaaggacaactctaagaat |
| | | caggtgtcactgaaactgtcatctgtgaccgcagccgacaccgccgtgtactattg |
| | | cgctaagcattactattatggcgggagctacgcaatggattactggggacagggta |
| | | ctctggtcaccgtgtccagcaccactaccccagcaccgaggccacccaccccggct |
| | | cctaccatcgcctcccagcctctgtccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 104880 | 98 | MALPVTALLLPLALLLHAARPeivmtqspatlslspgeratlscrasqdiskylnw |
| CAR6- | | yqqkpgqaprlliyhtsrlhsgiparfsgsgsgtdytltisslqpedfavyfcqqg |
| Full-aa | | ntlpytfgqgtkleikggggsggggsggggsggggsqvqlqesgpglvkpsetlsl |
| | | tctvsgvslpdygvswirqppgkglewigviwgsettyyqsslksrvtiskdnsks |
| | | qvslklssvtaadtavyycakhyyyggsyamdywgqgtivtvsstttpaprpptpa |
| | | ptiasqplslrpeacrpaaggavhtrgldfacdiyiwaplagtcgylllslvitly |
| | | ckrgrkkllyifkufmrpvqttcleedgcscrfpeeeeggcelrykfsrsadapay |
| | | kqgqnqlynelnlgrreeydvldkrrgrdpemggkprrknpqeglynelqkdkmae |
| | | ayseigmkgerrrgkghdglyqglstatkdtydalhmcialppr |

CAR 7

| | | |
|---|---|---|
| CAR7 | 51 | qvqlqesgpglvkpsetlsltctvsgvslpdygvswirqppgkglewigviwgset |
| scFv | | tyyssslksrvtiskdnsknqvslklssvtaadtavyycakhyyyggsyamdywgq |
| domain | | gtlvtvssggggsggggsggggsggggseivmtqspatlslspgeratlscrasqd |
| | | iskylnwyqqkpgqaprlliyhtsrlhsgiparfsgsgsgtdytltisslqpedfa |
| | | vyfcqqgntlpytfgqgtkleik |
| 100796 | 63 | atggcactgcctgtcactgccctcctgctgcctctggccctccttctgcatgccgc |
| CAR7- | | caggccccaagtccagctgcaagagtcaggacccggactggtgaagccgtctgaga |
| Soluble | | ctctctcactgacttgtaccgtcagcggcgtgtccctccccgactacggagtgtca |
| scFv-nt | | tggatccgccaacctcccgggaaagggcttgaatggattggtgtcatctggggttc |
| | | tgaaaccacctactactcatcttccctgaagtccagggtgaccatcagcaaggata |
| | | attccaagaaccaggtcagccttaagctgtcatctgtgaccgctgctgacaccgcc |
| | | gtgtattactgcgccaagcactactattacggaggaagctacgctatggactattg |
| | | gggacagggcactctcgtgactgtgagcagcggcggtggagggtctggaggtggag |
| | | gatccggtggtggtgggtcaggcggaggagggagcgagattgtgatgactcagtca |
| | | ccagccaccctttctctttcacccggcgagagagcaaccctgagctgtagagccag |
| | | ccaggacatttctaagtacctcaactggtatcagcaaaaaccggggcaggcccctc |
| | | gcctcctgatctaccatacctcacgccttcactctggtatccccgctcggtttagc |
| | | ggatcaggatctggtaccgactacactctgaccatttccagcctgcagccagaaga |
| | | tttcgcagtgtatttctgccagcagggcaatacccttccttacaccttcggtcagg |
| | | gaaccaagctcgaaatcaagcaccatcaccatcatcaccaccat |
| 100796 | 75 | MALPVTALLLPLALLLHAARPqvqlqesgpglvkpsetlsltctvsgvslpdygvs |
| CAR7- | | wirqppgkglewigviwgsettyyssslksrvtiskdnsknqvslklssvtaadta |
| Soluble | | vyycakhyyyggsyamdywgqgtivtvssggggsggggsggggsggggseivmtqs |
| scFv-aa | | patlslspgeratlscrasqdiskylnwyqqkpgqaprlliyhtsrlhsgiparfs |
| | | gsgsgtdytltisslqpedfavyfcqqgntlpytfgqgtkleikhhhhhhhh |
| 104881 | 87 | atggctctgcccgtgaccgcactcctcctgccactggctctgctgcttcacgccgc |
| CAR 7 | | tcgcccacaagtccagcttcaagaatcagggcctggtctggtgaagccatctgaga |
| Full-nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactattcatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccggaggtggcggaagcgaaatcgtgatgacccagagc |
| | | cctgcaaccctgtccctttctcccggggaacgggctaccctttcttgtcgggcatc |
| | | acaagatatctcaaaatacctcaattggtatcaacagaagccgggacaggccccta |
| | | ggcttcttatctaccacacctctcgcctgcatagcgggattcccgcacgctttagc |
| | | gggtctggaagcgggaccgactacactctgaccatctcatctctccagcccgagga |
| | | cttcgccgtctacttctgccagcagggtaacaccctgccgtacaccttcggccagg |
| | | gcaccaagcttgagatcaaaaccactactcccgctccaaggccacccacccctgcc |
| | | ccgaccatcgcctctcagccgctttccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 104881 | 99 | MALPVTALLLPLALLLHAARPqvqlqesgpglvkpsetlsltctvsgvslpdygvs |
| CAR 7 | | wirqppgkglewigviwgsettyyssslksrvtiskdnsknqvslklssvtaadta |
| Full-aa | | vyycakhyyyggsyamdywgqgtivtvssggggsggggsggggsggggseivmtqs |
| | | patlslspgeratlscrasqdiskylnwyqqkpgqaprlliyhtsrlhsgiparfs |
| | | gsgsgtdytltisslqpedfavyfcqqgntlpytfgqgtkleiktttpaprpptpa |
| | | ptiasqplslrpeacrpaaggavhtrgldfacdiyiwaplagtcgvlllslvitly |
| | | ckrgrkkllyifkqpfmrpvqttgeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR8

| | | |
|---|---|---|
| CAR8 | 52 | qvqlqesgpglvkpsetlsltctvsgvslpdygvswirqppgkglewigviwgset |
| scFv | | tyyqsslksrvtiskdnsknqvslklssvtaadtavyycakhyyyggsyamdywgq |
| domain | | gtivtvssggggsggggsggggsggggseivmtqspatlslspgeratlscrasqd |
| | | iskylnwyqqkpgqaprlliyhtsrlhsgiparfsgsgsgtdytltisslqpedfa |
| | | vyfcqqgntlpytfgqgtkleik |
| 100798 | 64 | atggcactgcctgtcactgccctcctgctgcctctggccctccttctgcatgccgc |
| CAR8- | | caggccccaagtccagctgcaagagtcaggacccggactggtgaagccgtctgaga |
| Soluble | | ctctctcactgacttgtaccgtcagcggcgtgtccctccccgactacggagtgtca |
| scFv-nt | | tggatccgccaacctcccgggaaagggcttgaatggattggtgtcatctggggttc |
| | | tgaaaccacctactaccagtcttccctgaagtccagggtgaccatcagcaaggata |
| | | attccaagaaccaggtcagccttaagctgtcatctgtgaccgctgctgacaccgcc |
| | | gtgtattactgcgccaagcactactattacggaggaagctacgctatggactattg |
| | | gggacagggcactctcgtgactgtgagcagcggcggtggagggtctggaggtggag |
| | | gatccggtggtggtgggtcaggcggaggagggagcgagattgtgatgactcagtca |
| | | ccagccaccctttctctttcacccggcgagagagcaaccctgagctgtagagccag |
| | | ccaggacatttctaagtacctcaactggtatcagcaaaaaccggggcaggcccctc |
| | | gcctcctgatctaccatacctcacgccttcactctggtatccccgctcggtttagc |
| | | ggatcaggatctggtaccgactacactctgaccatttccagcctgcagccagaaga |
| | | tttcgcagtgtatttctgccagcagggcaatacccttccttacaccttcggtcagg |
| | | gaaccaagctcgaaatcaagcaccatcaccatcatcatcaccac |
| 100798 | 76 | MALPVTALLLPLALLLHAARPqvqlqesgpglvkpsetlsltctvsgvslpdygvs |
| CAR8- | | wirqppgkglewigviwgsettyyqsslksrvtiskdnsknqvslklssvtaadta |
| Soluble | | vyycakhyyyggsyamdywgqgtivtvssggggsggggsggggsggggseivmtqs |
| scFv-aa | | patlslspgeratlscrasqdiskylnwyqqkpgqaprlliyhtsrlhsgiparfs |
| | | gsgsgtdytltisslqpedfavyfcqqgntlpytfgqgtkleikhhhhhhhh |
| 104882 | 88 | atggctctgcccgtgaccgcactcctcctgccactggctctgctgcttcacgccgc |
| CAR 8- | | tcgcccacaagtccagcttcaagaatcagggcctggtctggtgaagccatctgaga |
| Full-nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactatcaatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccggaggcggtgggtcagaaatcgtgatgacccagagc |
| | | cctgcaaccctgtccctttctcccggggaacgggctaccctttcttgtcgggcatc |
| | | acaagatatctcaaaatacctcaattggtatcaacagaagccgggacaggccccta |
| | | ggcttcttatctaccacacctctcgcctgcatagcgggattcccgcacgctttagc |
| | | gggtctggaagcgggaccgactacactctgaccatctcatctctccagcccgagga |
| | | cttcgccgtctacttctgccagcagggtaacaccctgccgtacaccttcggccagg |
| | | gcaccaagcttgagatcaaaaccactactcccgctccaaggccacccacccctgcc |
| | | ccgaccatcgcctctcagccgctttccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 104882 | 100 | MALPVTALLLPLALLLHAARPqvqlqesgpglvkpsetlsltctvsgvslpdygvs |
| CAR 8- | | wirqppgkglewigviwgsettyygsslksrvtiskdnsknqvslklssvtaadta |
| Full-aa | | vyycakhyyyggsyamdywgqgtivtvssggggsggggsggggsggggseivmtqs |
| | | patlslspgeratlscrasqdiskylnwyqqkpgqaprlliyhtsrlhsgiparfs |
| | | gsgsgtdytltisslqpedfavyfcqqgntlpytfgqgtkleiktttpaprpptpa |
| | | ptiasqplslrpeacrpaaggavhtrgldfacdiyiwaplagtcgvlllslvitly |
| | | ckrgrkkllyifkqpfmrpvqttgeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR 9

| | | |
|---|---|---|
| CAR9 | 53 | eivmtqspatlslspgeratlscrasqdiskylnwyqqkpgqaprlliyhtsrlhs |
| scFv | | giparfsgsgsgtdytltisslqpedfavyfcqqgntlpytfgqgtkleikggggs |
| domain | | ggggsggggsggggsqvqlqesgpglvkpsetlsltctvsgvslpdygvswirqpp |
| | | gkglewigviwgsettyynsslksrvtiskdnsknqvslklssvtaadtavyycak |
| | | hyyyggsyamdywgqgtlvtvss |
| 99789 | 65 | atggccctcccagtgaccgctctgctgctgcctctcgcacttcttctccatgccgc |
| CAR9- | | tcggcctgagatcgtcatgacccaaagccccgctaccctgtccctgtcacccggcg |
| Soluble | | agagggcaaccctttcatgcagggccagccaggacatttctaagtacctcaactgg |
| scFv-nt | | tatcagcagaagccagggcaggctcctcgcctgctgatctaccacaccagccgcct |
| | | ccacagcggtatccccgccagattttccgggagcgggtctggaaccgactacaccc |
| | | tcaccatctcttctctgcagcccgaggatttcgccgtctatttctgccagcagggg |
| | | aatactctgccgtacaccttcggtcaaggtaccaagctggaaatcaagggaggcgg |
| | | aggatcaggcggtggcggaagcggaggaggtggctccggaggaggaggttcccaag |
| | | tgcagcttcaagaatcaggacccggacttgtgaagccatcagaaaccctctccctg |
| | | acttgtaccgtgtccggtgtgagcctccccgactacggagtctcttggattcgcca |
| | | gcctccggggaagggtcttgaatggattggggtgatttggggatcagagactactt |
| | | actacaattcatcacttaagtcacgggtcaccatcagcaaagataatagcaagaac |
| | | caagtgtcacttaagctgtcatctgtgaccgccgctgacaccgccgtgtactattg |
| | | tgccaaacattactattacggagggtcttatgctatggactactggggacagggga |
| | | ccctggtgactgtctctagccatcaccatcaccaccatcatcac |
| 99789 | 77 | MALPVTALLLPLALLLHAARPeivmtqspatlslspgeratlscrasqdiskylnw |
| CAR9- | | yqqkpgqaprlliyhtsrlhsgiparfsgsgsgtdytltisslqpedfavyfcqqg |
| Soluble | | ntlpytfgqgtkleikggggsggggsggggsggggsqvqlqesgpglvkpsetlsl |
| scFv-aa | | tctvsgvslpdygvswirqppgkglewigviwgsettyynsslksrvtiskdnskn |
| | | qvslklssvtaadtavyycakhyyyggsyamdywgqgtivtvsshhhhhhhh |
| 105974 | 89 | atggccctccctgtcaccgccctgctgcttccgctggctcttctgctccacgccgc |
| CAR9- | | tcggcccgaaattgtgatgacccagtcacccgccactcttagcctttcacccggtg |
| Full-nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagcggaggcggtgggagccagg |
| | | tccaactccaagaaagcggaccgggtcttgtgaagccatcagaaactctttcactg |
| | | acttgtactgtgagcggagtgtctctccccgattacggggtgtcttggatcagaca |
| | | gccaccggggaagggtctggaatggattggagtgatttggggctctgagactactt |
| | | actacaactcatccctcaagtcacgcgtcaccatctcaaaggacaactctaagaat |
| | | caggtgtcactgaaactgtcatctgtgaccgcagccgacaccgccgtgtactattg |
| | | cgctaagcattactattatggcgggagctacgcaatggattactggggacagggta |
| | | ctctggtcaccgtgtccagcaccactaccccagcaccgaggccacccaccccggct |
| | | cctaccatcgcctcccagcctctgtccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 105974 | 101 | MALPVTALLLPLALLLHAARPeivmtqspatlslspgeratlscrasqdiskylnw |
| CAR 9- | | yqqkpgqaprlliyhtsrlhsgiparfsgsgsgtdytltisslqpedfavyfcqqg |
| Full-aa | | ntlpytfgqgtkleikggggsggggsggggsggggsqvcilqesgpglvkpsetlsl |
| | | tctvsgvslpdygvswirqppgkglewigviwgsettyynsslksrvtiskdnsks |
| | | qvslklssvtaadtavyycakhyyyggsyamdywgqgtivtvsstttpaprpptpa |
| | | ptiasqplslrpeacrpaaggavhtrgldfacdiyiwaplagtcgylllslvitly |
| | | ckrgrkkllyifkufmrpvqttcleedgcscrfpeeeeggcelrykfsrsadapay |
| | | kqgqnqlynelnlgrreeydvldkrrgrdpemggkprrknpqeglynelqkdkmae |
| | | ayseigmkgerrrgkghdglyqglstatkdtydalhmcialppr |

CAR10

| | | |
|---|---|---|
| CAR10 | 54 | qvqlqesgpglvkpsetlsltctvsgvslpdygvswirqppgkglewigviwgset |
| scFv | | tyynsslksrvtiskdnsknqvslklssvtaadtavyycakhyyyggsyamdywgq |
| domain | | gtlvtvssggggsggggsggggsggggseivmtqspatlslspgeratlscrasqd |
| | | iskylnwyqqkpgqaprlliyhtsrlhsgiparfsgsgsgtdytltisslqpedfa |
| | | vyfcqqgntlpytfgqgtkleik |
| 100796 | 66 | atggcactgcctgtcactgccctcctgctgcctctggccctccttctgcatgccgc |
| CAR10- | | caggccccaagtccagctgcaagagtcaggacccggactggtgaagccgtctgaga |
| Soluble | | ctctctcactgacttgtaccgtcagcggcgtgtccctccccgactacggagtgtca |
| scFv-nt | | tggatccgccaacctcccgggaaagggcttgaatggattggtgtcatctggggttc |
| | | tgaaaccacctactacaactcttccctgaagtccagggtgaccatcagcaaggata |
| | | attccaagaaccaggtcagccttaagctgtcatctgtgaccgctgctgacaccgcc |
| | | gtgtattactgcgccaagcactactattacggaggaagctacgctatggactattg |
| | | gggacagggcactctcgtgactgtgagcagcggcggtggagggtctggaggtggag |
| | | gatccggtggtggtgggtcaggcggaggagggagcgagattgtgatgactcagtca |
| | | ccagccaccctttctctttcacccggcgagagagcaaccctgagctgtagagccag |
| | | ccaggacatttctaagtacctcaactggtatcagcaaaaaccggggcaggcccctc |
| | | gcctcctgatctaccatacctcacgccttcactctggtatccccgctcggtttagc |
| | | ggatcaggatctggtaccgactacactctgaccatttccagcctgcagccagaaga |
| | | tttcgcagtgtatttctgccagcagggcaatacccttccttacaccttcggtcagg |
| | | gaaccaagctcgaaatcaagcaccatcaccatcatcaccaccat |
| 100796 | 78 | MALPVTALLLPLALLLHAARPqvqlqesgpglvkpsetlsltctvsgvslpdygvs |
| CAR10- | | wirqppgkglewigviwgsettyynsslksrvtiskdnsknqvslklssvtaadta |
| Soluble | | vyycakhyyyggsyamdywgqgtlvtvssggggsggggsggggsggggseivmtqs |
| scFv-aa | | patlslspgeratlscrasqdiskylnwyqqkpgqaprlliyhtsrlhsgiparfs |
| | | gsgsgtdytltisslqpedfavyfcqqgntlpytfgqgtkleikhhhhhhhh |
| 105975 | 90 | atggccctccctgtcaccgccctgctgcttccgctggctcttctgctccacgccgc |
| CAR 10 | | tcggcccgaaattgtgatgacccagtcacccgccactcttagcctttcacccggtg |
| Full-nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagcggaggcggtgggagccagg |
| | | tccaactccaagaaagcggaccgggtcttgtgaagccatcagaaactctttcactg |
| | | acttgtactgtgagcggagtgtctctccccgattacggggtgtcttggatcagaca |
| | | gccaccggggaagggtctggaatggattggagtgatttggggctctgagactactt |
| | | actacaactcatccctcaagtcacgcgtcaccatctcaaaggacaactctaagaat |
| | | caggtgtcactgaaactgtcatctgtgaccgcagccgacaccgccgtgtactattg |
| | | cgctaagcattactattatggcgggagctacgcaatggattactggggacagggta |
| | | ctctggtcaccgtgtccagcaccactaccccagcaccgaggccacccaccccggct |
| | | cctaccatcgcctcccagcctctgtccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 105975 | 102 | MALPVTALLLPLALLLHAARPEIVMTQSPATLSLSPGERATLSCRASQDISKYLNW |
| CAR 10 | | YQQKPGQAPRLLIYHTSRLHSGIPARFSGSGSGTDYTLTISSLQPEDFAVYFCQQG |
| Full-aa | | NTLPYTFGQGTKLEIKGGGGSGGGGSGGGGSGGGGSQVQLQESGPGLVKPSETLSL |
| | | TCTVSGVSLPDYGVSWIRQPPGKGLEWIGVIWGSETTYYNSSLKSRVTISKDNSKN |
| | | QVSLKLSSVTAADTAVYYCAKHYYYGGSYAMDYWGQGTLVTVSSTTTPAPRPPTPA |
| | | PTIASQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |

CAR11

| | | |
|---|---|---|
| CAR11 | 55 | eivmtqspatlslspgeratlscrasqdiskylnwyqqkpgqaprlliyhtsrlhs |
| scFv | | giparfsgsgsgtdytltisslqpedfavyfcqqgntlpytfgqgtkleikggggs |
| domain | | ggggsggggsqvqlqesgpglvkpsetlsltctvsgvslpdygvswirqppgkgle |
| | | wigviwgsettyynsslksrvtiskdnsknqvslklssvtaadtavyycakhyyyg |
| | | gsyamdywgqgtlvtvss |
| 103101 | 67 | Atggccctccctgtcaccgccctgctgcttccgctggctcttctgctccacgccgc |
| CAR11- | | tcggcccgaaattgtgatgacccagtcacccgccactcttagcctttcacccggtg |
| Soluble | | agcgcgcaaccctgtcttgcagagcctcccaagacatctcaaaataccttaattgg |
| scFv-nt | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactacaattcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagccaccaccatcatcaccatcaccat |
| 103101 | 79 | MALPVTALLLPLALLLHAARPeivmtqspatlslspgeratlscrasqdiskylnw |
| CAR11- | | yqqkpgqaprlliyhtsrlhsgiparfsgsgsgtdytltisslqpedfavyfcqqg |
| Soluble | | ntlpytfgqgtkleikggggsggggsggggsqvqlqesgpglvkpsetlsltctvs |
| scFv-aa | | gvslpdygvswirqppgkglewigviwgsettyynsslksrvtiskdnsknqvslk |
| | | lssvtaadtavyycakhyyyggsyamdywgqgtivtvsshhhhhhhh |
| 105976 | 91 | atggctctgcccgtgaccgcactcctcctgccactggctctgctgcttcacgccgc |
| CAR 11 | | tcgcccacaagtccagcttcaagaatcagggcctggtctggtgaagccatctgaga |
| Full-nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactataactcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccggaggtggcggaagcgaaatcgtgatgacccagagc |
| | | cctgcaaccctgtccctttctcccggggaacgggctaccctttcttgtcgggcatc |
| | | acaagatatctcaaaatacctcaattggtatcaacagaagccgggacaggccccta |
| | | ggcttcttatctaccacacctctcgcctgcatagcgggattcccgcacgctttagc |
| | | gggtctggaagcgggaccgactacactctgaccatctcatctctccagcccgagga |
| | | cttcgccgtctacttctgccagcagggtaacaccctgccgtacaccttcggccagg |
| | | gcaccaagcttgagatcaaaaccactactcccgctccaaggccacccacccctgcc |
| | | ccgaccatcgcctctcagccgctttccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 105976 | 103 | MALPVTALLLPLALLLHAARPQVQLQESGPGLVKPSETLSLTCTVSGVSLPDYGVS |
| CAR 11 | | WIRQPPGKGLEWIGVIWGSETTYYNSSLKSRVTISKDNSKNQVSLKLSSVTAADTA |
| Full-aa | | VYYCAKHYYYGGSYAMDYWGQGTLVTVSSGGGGSGGGGSGGGGSGGGGSEIVMTQS |
| | | PATLSLSPGERATLSCRASQDISKYLNWYQQKPGQAPRLLIYHTSRLHSGIPARFS |
| | | GSGSGTDYTLTISSLQPEDFAVYFCQQGNTLPYTFGQGTKLEIKTTTPAPRPPTPA |
| | | PTIASQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |

CAR12

| | | |
|---|---|---|
| CAR12 | 56 | qvqlqesgpglvkpsetlsltctvsgvslpdygvswirqppgkglewigviwgset |
| scFv | | tyynsslksrvtiskdnsknqvslklssvtaadtavyycakhyyyggsyamdywgq |
| domain | | gtivtvssggggsggggsggggseivmtqspatlslspgeratlscrasqdiskyl |
| | | nwyqqkpgqaprlliyhtsrlhsgiparfsgsgsgtdytltisslqpedfavyfcq |
| | | qgntlpytfgqgtkleik |
| 103104 | 68 | atggctctgcccgtgaccgcactcctcctgccactggctctgctgcttcacgccgc |
| CAR12- | | tcgcccacaagtccagcttcaagaatcagggcctggtctggtgaagccatctgaga |
| Soluble | | ctctgtccctcacttgcaccgtgagcggagtgtccctcccagactacggagtgagc |
| scFv-nt | | tggattagacagcctcccggaaagggactggagtggatcggagtgatttggggtag |
| | | cgaaaccacttactataactcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaacatcaccaccatcatcaccatcac |
| 103104 | 80 | MALPVTALLLPLALLLHAARPqvqlqesgpglvkpsetlsltctvsgvslpdygys |
| CAR12- | | wirqppgkglewigviwgsettyynsslksrvtiskdnsknqvslklssvtaadta |
| Soluble | | vyycakhyyyggsyamdywgqgtivtvssggggsggggsggggseivmtqspatls |
| seFv-aa | | lspgeratlscrasqdiskylnwyqqkpgqaprlliyhtsrlhsgiparfsgsgsg |
| | | tdytltisslqpedfavyfcqqgntlpytfgqgtkleikhhhhhhhh |
| 105977 | 92 | atggccctccctgtcaccgccctgctgcttccgctggctcttctgctccacgccgc |
| CAR12- | | tcggcccgaaattgtgatgacccagtcacccgccactcttagcctttcacccggtg |
| Full-nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactacaactcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagcaccactaccccagcaccgaggccacccaccccggctcctaccatcgcctcc |
| | | cagcctctgtccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 105977 | 104 | MALPVTALLLPLALLLHAARPEIVMTQSPATLSLSPGERATLSCRASQDISKYLNW |
| CAR12- | | YQQKPGQAPRLLIYHTSRLHSGIPARFSGSGSGTDYTLTISSLQPEDFAVYFCQQG |
| Full-aa | | NTLPYTFGQGTKLEIKGGGGSGGGGSGGGGSQVQLQESGPGLVKPSETLSLTCTVS |
| | | GVSLPDYGVSWIRQPPGKGLEWIGVIWGSETTYYNSSLKSRVTISKDNSKNQVSLK |
| | | LSSVTAADTAVYYCAKHYYYGGSYAMDYWGQGTLVTVSSTTTPAPRPPTPAPTIAS |
| | | QPLSLRPEACRPAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRSADAPAYKQGQN |
| | | QLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR |

For all soluble scFv amino acid sequences, an optional signal sequence is shown in bold and underline; and the histidine tag is underlined.
For all CAR amino acid sequences, the relative location of the CDRs is underlined and bold.

TABLE 3
Murine CD19 CAR Constructs

| | SEQ ID | |
|---|---|---|
| CTL019 | NO: | |
| CTL019- | 105 | Atggccctgcccgtcaccgctctgctgctgccccttgctctgcttcttcatgcag |
| Soluble | | caaggccggacatccagatgacccaaaccacctcatccctctctgcctctcttgg |
| scFv- | | agacagggtgaccatttcttgtcgcgccagccaggacatcagcaagtatctgaac |
| Histag - nt | | tggtatcagcagaagccggacggaaccgtgaagctcctgatctaccatacctctc |
| | | gcctgcatagcggcgtgccctcacgcttctctggaagcggatcaggaaccgatta |
| | | ttctctcactatttcaaatcttgagcaggaagatattgccacctatttctgccag |
| | | cagggtaataccctgccctacaccttcggaggagggaccaagctcgaaatcaccg |
| | | gtggaggaggcagcggcggtggagggtctggtggaggtggttctgaggtgaagct |
| | | gcaagaatcaggccctggacttgtggccccttcacagtccctgagcgtgacttgc |
| | | accgtgtccggagtctccctgcccgactacggagtgtcatggatcagacaacctc |
| | | cacggaaaggactggaatggctcggtgtcatctggggtagcgaaactacttacta |
| | | caattcagccctcaaaagcaggctgactattatcaaggacaacagcaagtcccaa |
| | | gtctttcttaagatgaactcactccagactgacgacaccgcaatctactattgtg |
| | | ctaagcactactactacggaggatcctacgctatggattactggggacaaggtac |
| | | ttccgtcactgtctcttcacaccatcatcaccatcaccatcac |
| CTL019- | 106 | MALPVTALLLPLALLLHAARPdiqmtqttsslsaslgdrvtiscrasqdiskyln |
| Soluble | | wyqqkpdgtvklliyhtsrlhsgvpsrfsgsgsgtdysltisnleqediatyfcq |
| scFv- | | qgntlpytfgggtkleitggggsggggsggggsevklqesgpglvapsqslsvtc |
| Histag - aa | | tvsgvslpdygyswirqpprkglewlgviwgsettyynsalksrltiikdnsksq |
| | | vflkmnslqtddtaiyycakhyyyggsyamdywgqgtsvtvsshhhhhhhh |
| CTL019 | 107 | atggccttaccagtgaccgccttgctcctgccgctggccttgctgctccacgccg |
| Full - nt | | ccaggccggacatccagatgacacagactacatcctccctgtctgcctctctggg |
| | | agacagagtcaccatcagttgcagggcaagtcaggacattagtaaatatttaaat |
| | | tggtatcagcagaaaccagatggaactgttaaactcctgatctaccatacatcaa |
| | | gattacactcaggagtcccatcaaggttcagtggcagtgggtctggaacagatta |
| | | ttctctcaccattagcaacctggagcaagaagatattgccacttacttttgccaa |
| | | cagggtaatacgcttccgtacacgttcggaggggggaccaagctggagatcacag |
| | | gtggcggtggctcgggcggtggtgggtcgggtggcggcggatctgaggtgaaact |
| | | gcaggagtcaggacctggcctggtggcgccctcacagagcctgtccgtcacatgc |
| | | actgtctcaggggtctcattacccgactatggtgtaagctggattcgccagcctc |
| | | cacgaaagggtctggagtggctgggagtaatatggggtagtgaaaccacatacta |
| | | taattcagctctcaaatccagactgaccatcatcaaggacaactccaagagccaa |
| | | gttttcttaaaaatgaacagtctgcaaactgatgacacagccatttactactgtg |
| | | ccaaacattattactacggtggtagctatgctatggactactggggccaaggaac |
| | | ctcagtcaccgtctcctcaaccacgacgccagcgccgcgaccaccaacaccggcg |
| | | cccaccatcgcgtcgcagcccctgtccctgcgcccagaggcgtgccggccagcgg |
| | | cggggggcgcagtgcacacgagggggctggacttcgcctgtgatatctacatctg |
| | | ggcgcccttggccgggacttgtggggtccttctcctgtcactggttatcaccctt |
| | | tactgcaaacggggcagaaagaaactcctgtatatattcaaacaaccatttatga |
| | | gaccagtacaaactactcaagaggaagatggctgtagctgccgatttccagaaga |
| | | agaagaaggaggatgtgaactgagagtgaagttcagcaggagcgcagacgccccc |
| | | gcgtacaagcagggccagaaccagctctataacgagctcaatctaggacgaagag |
| | | aggagtacgatgttttggacaagagacgtggccgggaccctgagatggggggaaa |
| | | gccgagaaggaagaaccctcaggaaggcctgtacaatgaactgcagaaagataag |
| | | atggcggaggcctacagtgagattgggatgaaaggcgagcgccggaggggcaagg |
| | | ggcacgatggcctttaccagggtctcagtacagccaccaaggacacctacgacgc |
| | | ccttcacatgcaggccctgccccctcgc |
| CTL019 | 108 | MALPVTALLLPLALLLHAARPdiqmtqttsslsaslgdrvtiscrasqdiskyln |
| Full - aa | | wyqqkpdgtvklliyhtsrlhsgvpsrfsgsgsgtdysltisnleqediatyfcq |
| | | qgntlpytfgggtkleitggggsggggsggggsevklqesgpglvapsqslsvtc |
| | | tvsgvslpdygvswirqpprkglewlgviwgsettyynsalksrltiikdnsksq |
| | | vflkmnslqtddtaiyycakhyyyggsyamdywgqgtsvtvsstttpaprpptpa |
| | | ptiasqplslrpeacrpaaggavhtrgldfacdiyiwaplagtcgvlllslvitl |
| | | yckrgrkkllyifkqpfmrpvqttqeedgcscrfpeeeeggcelrvkfsrsadap |
| | | aykqgqnqlynelnlgrreeydvldkrrgrdpemggkprrknpqeglynelqkdk |
| | | maeayseigmkgerrrgkghdglyqglstatkdtydalhmqalppr |
| CTL019 | 109 | Diqmtqttsslsaslgdrvtiscrasqdiskylnwyqqkpdgtvklliyhtsrlh |
| scFv | | sgvpsrfsgsgsgtdysltisnleqediatyfcqqgntlpytfgggtkleitggg |
| domain | | gsggggsggggsevklqesgpglvapsqslsvtctvsgvslpdygvswirqpprk |
| | | glewlgviwgsettyynsalksrltiikdnsksqvflkmnslqtddtaiyycakh |
| | | yyyggsyamdywgqgtsvtvss |
| mCAR1 | 110 | QVQLLESGAELVRPGSSVKISCKASGYAFSSYWMNWVKQRPGQGLEWIGQIYPGD |
| scFv | | GDTNYNGKFKGQATLTADKSSSTAYMQLSGLTSEDSAVYSCARKTISSVVDFYFD |
| | | YWGQGTTVTGGGSGGGSGGGSGGGSELVLTQSPKFMSTSVGDRVSVTCKASQNVG |
| | | TNVAWYQQKPGQSPKPLIYSATYRNSGVPDRFTGSGSGTDFTLTITNVQSKDLAD |
| | | YFCQYNRYPYTSFFFTKLEIKRRS |
| mCAR1 | 111 | QVQLLESGAELVRPGSSVKISCKASGYAFSSYWMNWVKQRPGQGLEWIGQIYPGD |
| Full - aa | | GDTNYNGKFKGQATLTADKSSSTAYMQLSGLTSEDSAVYSCARKTISSVVDFYFD |
| | | YWGQGTTVTGGGSGGGSGGGSGGGSELVLTQSPKFMSTSVGDRVSVTCKASQNVG |
| | | TNVAWYQQKPGQSPKPLIYSATYRNSGVPDRFTGSGSGTDFTLTITNVQSKDLAD |
| | | YFCQYNRYPYTSFFFTKLEIKRRSKIEVMYPPPYLDNEKSNGTIIHVKGKHLCPS |
| | | PLFPGPSKPFWVLVVVGGVLACYSLLVTVAFIIFWVRSKRSRLLHSDYMNMTPRR |
| | | PGPTRKHYQPYAPPRDFAAYRSRVKFSRSADAPAYQQGQNQLYNELNLGRREEYD |
| | | VLDKRRGRDPEMGGKPRRKNPQEGLYNELQKDKMAEAYSEIGMKGERRRGKGHDG |
| | | LYQGLSTATKDTYDALHMQALPPR |
| mCAR2 | 112 | DIQMTQTTSSLSASLGDRVTISCRASQDISKYLNWYQQKPDGTVKLLIYHTSRLH |
| scFv | | SGVPSRFSGSGSGTDYSLTISNLEQEDIATYFCQQGNTLPYTFGGGTKLEITGST |
| | | SGSGKPGSGEGSTKGEVKLQESGPGLVAPSQSLSVTCTVSGVSLPDYGVSWIRQP |
| | | PRKGLEWLGVIWGSETTYYNSALKSRLTIIKDNSKSQVFLKMNSLQTDDTAIYYC |
| | | AKHYYYGGSYAMDYWGQGTSVTVSSE |
| mCAR2 | 113 | DIQMTQTTSSLSASLGDRVTISCRASQDISKYLNWYQQKPDGTVKLLIYHTSRLH |
| CAR- aa | | SGVPSRFSGSGSGTDYSLTISNLEQEDIATYFCQQGNTLPYTFGGGTKLEITGST |
| | | SGSGKPGSGEGSTKGEVKLQESGPGLVAPSQSLSVTCTVSGVSLPDYGVSWIRQP |
| | | PRKGLEWLGVIWGSETTYYNSALKSRLTIIKDNSKSQVFLKMNSLQTDDTAIY |
| | | YCAKHYYYGGSYAMDYWGQGTSVTVSSESKYGPPCPPCPMFWVLVVVGGVLACYS |
| | | LLVTVAFIIFWVKRGRKKLLYIFKQPFMRPVQTTQEEDGCSCRFEEEEGGCELRV |
| | | KFSRSADAPAYQQGQNQLYNELNLGRREEYDVLDKRRGRDPEMGGKPRRKNPQEG |
| | | LYNELQKDKMAEAYSEIGMKGERRRGKGHDGLYQGLSTATKDTYDALHMQALPPR |
| | | L |
| mCAR2 | 114 | DIQMTQTT SSLSASLGDR VTISCRASQD ISKYLNWYQQ KPDGTVKLLI |
| Full - aa | | YHTSRLHSGV PSRFSGSGSG TDYSLTISNL EQEDIATYFC QQGNTLPYTF |
| | | GGGTKLEITG STSGSGKPGS GEGSTKGEVK LQESGPGLVA PSQSLSVTCT |
| | | VSGVSLPDYG VSWIRQPPRK GLEWLGVIWG SETTYYNSAL KSRLTIIKDN |
| | | SKSQVFLKMN SLQTDDTAIY YCAKHYYYGG SYAMDYWGQG TSVTVSSESK |
| | | YGPPCPPCPM FWVLVVVGGV LACYSLLVTV |
| | | AFIIFWVKRG RKKLLYIFKQ PFMRPVQTTQ EEDGCSCRFE EEEGGCELRV |
| | | KFSRSADAPA YQQGQNQLYN ELNLGRREEY DVLDKRRGRD PEMGGKPRRK |
| | | NPQEGLYNEL QKDKMAEAYS EIGMKGERRR GKGHDGLYQG LSTATKDTYD |
| | | ALHMQALPPR LEGGGEGRGS LLTCGDVEEN PGPRMLLLVT SLLLCELPHP |
| | | AFLLIPRKVC NGIGIGEFKD SLSINATNIK HFKNCTSISG DLHILPVAFR |
| | | GDSFTHTPPL DPQELDILKT VKEITGFLLI QAWPENRTDL HAFENLEIIR |
| | | GRTKQHGQFS LAVVSLNITS LGLRSLKEIS DGDVIISGNK NLCYANTINW |
| | | KKLFGTSGQK TKIISNRGEN SCKATGQVCH ALCSPEGCWG PEPRDCVSCR |
| | | NVSRGRECVD KCNLLEGEPR EFVENSECIQ CHPECLPQAM NITCTGRGPD |
| | | NCIQCAHYID GPHCVKTCPA GVMGENNTLV WKYADAGHVC HLCHPNCTYG |
| | | CTGPGLEGCP TNGPKIPSIA TGMVGALLLL LVVALGIGLF M |
| mCAR3 | 115 | DIQMTQTTSSLSASLGDRVTISCRASQDISKYLNWYQQKPDGTVKLLIYHTSRLH |
| scFv | | SGVPSRFSGSGSGTDYSLTISNLEQEDIATYFCQQGNTLPYTFGGGTKLEITGST |
| | | SGSGKPGSGEGSTKGEVKLQESGPGLVAPSQSLSVTCTVSGVSLPDYGVSWIRQP |
| | | PRKGLEWLGVIWGSETTYYNSALKSRLTIIKDNSKSQVFLKMNSLQTDDTAIYYC |
| | | AKHYYYGGSYAMDYWGQGTSVTVSS |
| mCAR3 | 116 | DIQMTQTTSSLSASLGDRVTISCRASQDISKYLNWYQQKPDGTVKLLIYHTSRLH |
| Full - aa | | SGVPSRFSGSGSGTDYSLTISNLEQEDIATYFCQQGNTLPYTFGGGTKLEITGST |
| | | SGSGKPGSGEGSTKGEVKLQESGPGLVAPSQSLSVTCTVSGVSLPDYGVSWIRQP |
| | | PRKGLEWLGVIWGSETTYYNSALKSRLTIIKDNSKSQVFLKMNSLQTDDTAIYYC |
| | | AKHYYYGGSYAMDYWGQGTSVTVSSAAAIEVMYPPPYLDNEKSNGTIIHVKGKHL |
| | | CPSPLFPGPSKPFWVLVVVGGVLACYSLLVTVAFIIFWVRSKRSRLLHSDYMNMT |
| | | PRRPGPTRKHYQPYAPPRDFAAYRSRVKFSRSADAPAYQQGQNQLYNELNLGRRE |
| | | EYDVLDKRRGRDPEMGGKPRRKNPQEGLYNELQKDKMAEAYSEIGMKGERRRGKG |
| | | HDGLYQGLSTATKDTYDALHMQALPPR |
| SSJ25-C1 | 287 | QVQLLESGAELVRPGSSVKISCKASGYAFSSYWMNWVKQRPGQGLEWIGQIYPGD |
| VH | | GDTNYNGKFKGQATLTADKSSSTAYMQLSGLTSEDSAVYSCARKTISSVVDFYFD |
| sequence | | YWGQGTTVT |
| SSJ25-C1 | 288 | ELVLTQSPKFMSTSVGDRVSVTCKASQNVGTNVAWYQQKPGQSPKPLIYSATYRN |
| VL | | SGVPDRFTGSGSGTDFTLTITNVQSKDLADYFYFCQYNRYPYTSGGGTKLEIKRR |
| sequence | | S |

For all soluble scFv amino acid sequences, an optional signal sequence is shown in bold and underline; and the histidine tag is underlined.

In some embodiments, the antigen binding domain comprises a HC CDR1, a HC CDR2, and a HC CDR3 of any heavy chain binding domain amino acid sequences listed in Table 2 or 3. In embodiments, the antigen binding domain further comprises a LC CDR1, a LC CDR2, and a LC CDR3. In embodiments, the antigen binding domain comprises a LC CDR1, a LC CDR2, and a LC CDR3 of any light chain binding domain amino acid sequences listed in Table 2 or 3.

In some embodiments, the antigen binding domain comprises one, two or all of LC CDR1, LC CDR2, and LC CDR3 of any light chain binding domain amino acid sequences listed in Table 2 or 3, and one, two or all of HC CDR1, HC CDR2, and HC CDR3 of any heavy chain binding domain amino acid sequences listed in Table 2 or 3.

In some embodiments, the CDRs are defined according to the Kabat numbering scheme, the Chothia numbering scheme, or a combination thereof.

The sequences of humanized CDR sequences of the scFv domains are shown in Table 4 for the heavy chain variable domains and in Table 5 for the light chain variable domains. “ID” stands for the respective SEQ ID NO for each CDR.

TABLE 4
Heavy Chain Variable Domain CDRs (Kabat)

| | | | SEQ | | SEQ | | SEQ |
|---|---|---|---|---|---|---|---|
| | | | ID | | ID | | ID |
| Candidate | FW | HCDR1 | NO: | HCDR2 | NO: | HCDR3 | NO: |
| murine_CART19 | | DYGVS | 117 | VIWGSETTYYNSALKS | 118 | HYYYGGSYAMDY | 122 |
| humanized_CART19 | VH4 | DYGVS | 117 | VIWGSETTYY [symbol] S [symbol] LKS | 119 | HYYYGGSYAMDY | 122 |
| a | | | | | | | |
| humanized_CART19 | VH4 | DYGVS | 117 | VIWGSETTYY [symbol] S [symbol] LKS | 120 | HYYYGGSYAMDY | 122 |
| b | | | | | | | |
| humanized_CART19 | VH4 | DYGVS | 117 | VIWGSETTYYNS [symbol] LKS | 121 | HYYYGGSYAMDY | 122 |
| c | | | | | | | |

TABLE 5
Light Chain Variable Domain CDRs (Kabat)

| | | | SEQ | | SEQ | | SEQ |
|---|---|---|---|---|---|---|---|
| | | | ID | | ID | | ID |
| Candidate | FW | LCDR1 | NO: | LCDR2 | NO: | LCDR3 | NO: |
| murine_CART19 | | RASQDISKYLN | 123 | HTSRLHS | 124 | QQGNTLPYT | 125 |
| humanized_CART19 a | VK3 | RASQDISKYLN | 123 | HTSRLHS | 124 | QQGNTLPYT | 125 |
| humanized_CART19 b | VK3 | RASQDISKYLN | 123 | HTSRLHS | 124 | QQGNTLPYT | 125 |
| humanized_CART19 c | VK3 | RASQDISKYLN | 123 | HTSRLHS | 124 | QQGNTLPYT | 125 |

The CAR scFv fragments were then cloned into lentiviral vectors to create a full length CAR construct in a single coding frame, and using the EF1 alpha promoter for expression (SEQ ID NO: 11).

In some embodiments, the CD19 CAR comprises an antigen binding domain derived from (e.g., comprises an amino acid sequence of) an anti-CD19 antibody (e.g., an anti-CD19 mono- or bispecific antibody) or a fragment or conjugate thereof. In one embodiment, the anti-CD19 antibody is a humanized antigen binding domain as described in WO2014/153270 (e.g., Table 3 of WO2014/153270) incorporated herein by reference, or a conjugate thereof. Other exemplary anti-CD19 antibodies or fragments or conjugates thereof, include but are not limited to, a bispecific T cell engager that targets CD19 (e.g., blinatumomab), SAR3419 (Sanofi), MEDI-551 (MedImmune LLC), Combotox, DT2219ARL (Masonic Cancer Center), MOR-208 (also called XmAb-5574; MorphoSys), XmAb-5871 (Xencor), MDX-1342 (Bristol-Myers Squibb), SGN-CD19A (Seattle Genetics), and AFM11 (Affimed Therapeutics). See, e.g., Hammer. MAbs. 4.5(2012): 571-77. Blinatomomab is a bispecific antibody comprised of two scFvs—one that binds to CD19 and one that binds to CD3. Blinatomomab directs T cells to attack cancer cells. See, e.g., Hammer et al.; Clinical Trial Identifier No. NCT00274742 and NCT01209286. MEDI-551 is a humanized anti-CD19 antibody with a Fc engineered to have enhanced antibody-dependent cell-mediated cytotoxicity (ADCC). See, e.g., Hammer et al.; and Clinical Trial Identifier No. NCT01957579. Combotox is a mixture of immunotoxins that bind to CD19 and CD22. The immunotoxins are made up of scFv antibody fragments fused to a deglycosylated ricin A chain. See, e.g., Hammer et al.; and Herrera et al. J. Pediatr. Hematol. Oncol. 31.12(2009):936-41; Schindler et al. Br. J. Haematol. 154.4(2011):471-6. DT2219ARL is a bispecific immunotoxin targeting CD19 and CD22, comprising two scFvs and a truncated diphtheria toxin. See, e.g., Hammer et al.; and Clinical Trial Identifier No. NCT00889408. SGN-CD19A is an antibody-drug conjugate (ADC) comprised of an anti-CD19 humanized monoclonal antibody linked to a synthetic cytotoxic cell-killing agent, monomethyl auristatin F (MMAF). See, e.g., Hammer et al.; and Clinical Trial Identifier Nos. NCT01786096 and NCT01786135. SAR3419 is an anti-CD19 antibody-drug conjugate (ADC) comprising an anti-CD19 humanized monoclonal antibody conjugated to a maytansine derivative via a cleavable linker. See, e.g., Younes et al. J. Clin. Oncol. 30.2(2012): 2776-82; Hammer et al.; Clinical Trial Identifier No. NCT00549185; and Blanc et al. Clin Cancer Res. 2011; 17:6448-58. XmAb-5871 is an Fc-engineered, humanized anti-CD19 antibody. See, e.g., Hammer et al. MDX-1342 is a human Fc-engineered anti-CD19 antibody with enhanced ADCC. See, e.g., Hammer et al. In embodiments, the antibody molecule is a bispecific anti-CD19 and anti-CD3 molecule. For instance, AFM11 is a bispecific antibody that targets CD19 and CD3. See, e.g., Hammer et al.; and Clinical Trial Identifier No. NCT02106091. In some embodiments, an anti-CD19 antibody described herein is conjugated or otherwise bound to a therapeutic agent, e.g., a chemotherapeutic agent, peptide vaccine (such as that described in Izumoto et al. 2008 J Neurosurg 108:963-971), immunosuppressive agent, or immunoablative agent, e.g., cyclosporin, azathioprine, methotrexate, mycophenolate, FK506, CAMPATH, anti-CD3 antibody, cytoxin, fludarabine, rapamycin, mycophenolic acid, steroid, FR901228, or cytokine.

In one embodiment, an antigen binding domain against CD19 is an antigen binding portion, e.g., CDRs, of an antigen binding domain described in a Table herein. In one embodiment, a CD19 antigen binding domain can be from any CD19 CAR, e.g., LG-740; U.S. Pat. No. 8,399,645; U.S. Pat. No. 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10, each of which is herein incorporated by reference in its entirety.

### Exemplary BCMA Antigen Binding Domains and CAR Constructs

In embodiments the BCMA CAR comprises an anti-BCMA binding domain (e.g., human or humanized anti-BCMA binding domain), a transmembrane domain, and an intracellular signaling domain, and wherein said anti-BCMA binding domain comprises a heavy chain complementary determining region 1 (HC CDR1), a heavy chain complementary determining region 2 (HC CDR2), and a heavy chain complementary determining region 3 (HC CDR3) of any anti-BMCA heavy chain binding domain amino acid sequences listed in Table 7 or 8.

In one embodiment, the anti-BCMA binding domain comprises a light chain variable region described herein (e.g., in Table 7 or 8) and/or a heavy chain variable region described herein (e.g., in Table 7 or 8).

In one embodiment, the encoded anti-BCMA binding domain is a scFv comprising a light chain and a heavy chain of an amino acid sequence of Table 7 or 8.

In an embodiment, the human or humanized anti-BCMA binding domain (e.g., an scFv) comprises: a light chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a light chain variable region provided in Table 7 or 8, or a sequence with at least 95% (e.g., 95-99%) identity thereof; and/or a heavy chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a heavy chain variable region provided in Table 7 or 8, or a sequence with at least 95% (e.g., 95-99%) identity thereof.

TABLE 7
Amino Acid and Nucleic Acid Sequences of exemplary anti-BCMA scFv domains
and BCMA CAR molecules

| | | |
|---|---|---|
| | SEQ | |
| Name/ | ID | |
| Description | NO: | Sequence |

139109

| | | |
|---|---|---|
| 139109- aa | 294 | EVQLVESGGGLVQPGGSLRLSCAVSGFALSNHGMSWVRRAPGKGLEWV |
| ScFv | | SGIVYSGSTYYAASVKGRFTISRDNSRNTLYLQMNSLRPEDTAIYYCS |
| domain | | AHGGESDVWGQGTTVTVSSASGGGGSGGRASGGGGSDIQLTQSPSSLS |
| | | ASVGDRVTITCRASQSISSYLNWYQQKPGKAPKLLIYAASSLQSGVPS |
| | | RFSGSGSGTDFTLTISSLQPEDFATYYCQQSYSTPYTFGQGTKVEIK |
| 139109- nt | 295 | GAAGTGCAATTGGTGGAATCAGGGGGAGGACTTGTGCAGCCTGGAGGA |
| ScFv | | TCGCTGAGACTGTCATGTGCCGTGTCCGGCTTTGCCCTGTCCAACCAC |
| domain | | GGGATGTCCTGGGTCCGCCGCGCGCCTGGAAAGGGCCTCGAATGGGTG |
| | | TCGGGTATTGTGTACAGCGGTAGCACCTACTATGCCGCATCCGTGAAG |
| | | GGGAGATTCACCATCAGCCGGGACAACTCCAGGAACACTCTGTACCTC |
| | | CAAATGAATTCGCTGAGGCCAGAGGACACTGCCATCTACTACTGCTCC |
| | | GCGCATGGCGGAGAGTCCGACGTCTGGGGACAGGGGACCACCGTGACC |
| | | GTGTCTAGCGCGTCCGGCGGAGGCGGCAGCGGGGGTCGGGCATCAGGG |
| | | GGCGGCGGATCGGACATCCAGCTCACCCAGTCCCCGAGCTCGCTGTCC |
| | | GCCTCCGTGGGAGATCGGGTCACCATCACGTGCCGCGCCAGCCAGTCG |
| | | ATTTCCTCCTACCTGAACTGGTACCAACAGAAGCCCGGAAAAGCCCCG |
| | | AAGCTTCTCATCTACGCCGCCTCGAGCCTGCAGTCAGGAGTGCCCTCA |
| | | CGGTTCTCCGGCTCCGGTTCCGGTACTGATTTCACCCTGACCATTTCC |
| | | TCCCTGCAACCGGAGGACTTCGCTACTTACTACTGCCAGCAGTCGTAC |
| | | TCCACCCCCTACACTTTCGGACAAGGCACCAAGGTCGAAATCAAG |
| 139109- aa | 296 | EVQLVESGGGLVQPGGSLRLSCAVSGFALSNHGMSWVRRAPGKGLEWV |
| VH | | SGIVYSGSTYYAASVKGRFTISRDNSRNTLYLQMNSLRPEDTAIYYCS |
| | | AHGGESDVWGQGTTVTVSS |
| 139109- aa | 297 | DIQLTQSPSSLSASVGDRVTITCRASQSISSYLNWYQQKPGKAPKLLI |
| VL | | YAASSLQSGVPSRFSGSGSGTDFTLTISSLQPEDFATYYCQQSYSTPY |
| | | TFGQGTKVEIK |
| 139109- aa | 298 | MALPVTALLLPLALLLHAARPEVQLVESGGGLVQPGGSLRLSCAVSGF |
| Full CAR | | ALSNHGMSWVRRAPGKGLEWVSGIVYSGSTYYAASVKGRFTISRDNSR |
| | | NTLYLQMNSLRPEDTAIYYCSAHGGESDVWGQGTTVTVSSASGGGGSG |
| | | GRASGGGGSDIQLTQSPSSLSASVGDRVTITCRASQSISSYLNWYQQK |
| | | PGKAPKLLIYAASSLQSGVPSRFSGSGSGTDFTLTISSLQPEDFATYY |
| | | CQQSYSTPYTFGQGTKVEIKTTTPAPRPPTPAPTIASQPLSLRPEACR |
| | | PAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQAL |
| | | PPR |
| 139109- nt | 299 | ATGGCCCTCCCTGTCACCGCCCTGCTGCTTCCGCTGGCTCTTCTGCTC |
| Full CAR | | CACGCCGCTCGGCCCGAAGTGCAATTGGTGGAATCAGGGGGAGGACTT |
| | | GTGCAGCCTGGAGGATCGCTGAGACTGTCATGTGCCGTGTCCGGCTTT |
| | | GCCCTGTCCAACCACGGGATGTCCTGGGTCCGCCGCGCGCCTGGAAAG |
| | | GGCCTCGAATGGGTGTCGGGTATTGTGTACAGCGGTAGCACCTACTAT |
| | | GCCGCATCCGTGAAGGGGAGATTCACCATCAGCCGGGACAACTCCAGG |
| | | AACACTCTGTACCTCCAAATGAATTCGCTGAGGCCAGAGGACACTGCC |
| | | ATCTACTACTGCTCCGCGCATGGCGGAGAGTCCGACGTCTGGGGACAG |
| | | GGGACCACCGTGACCGTGTCTAGCGCGTCCGGCGGAGGCGGCAGCGGG |
| | | GGTCGGGCATCAGGGGGCGGCGGATCGGACATCCAGCTCACCCAGTCC |
| | | CCGAGCTCGCTGTCCGCCTCCGTGGGAGATCGGGTCACCATCACGTGC |
| | | CGCGCCAGCCAGTCGATTTCCTCCTACCTGAACTGGTACCAACAGAAG |
| | | CCCGGAAAAGCCCCGAAGCTTCTCATCTACGCCGCCTCGAGCCTGCAG |
| | | TCAGGAGTGCCCTCACGGTTCTCCGGCTCCGGTTCCGGTACTGATTTC |
| | | ACCCTGACCATTTCCTCCCTGCAACCGGAGGACTTCGCTACTTACTAC |
| | | TGCCAGCAGTCGTACTCCACCCCCTACACTTTCGGACAAGGCACCAAG |
| | | GTCGAAATCAAGACCACTACCCCAGCACCGAGGCCACCCACCCCGGCT |
| | | CCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGC |
| | | GATATCTACATTTGGGCCCCTCTGGCTGGTACTTGCGGGGTCCTGCTG |
| | | CTTTCACTCGTGATCACTCTTTACTGTAAGCGCGGTCGGAAGAAGCTG |
| | | CTGTACATCTTTAAGCAACCCTTCATGAGGCCTGTGCAGACTACTCAA |
| | | GAGGAGGACGGCTGTTCATGCCGGTTCCCAGAGGAGGAGGAAGGCGGC |
| | | TGCGAACTGCGCGTGAAATTCAGCCGCAGCGCAGATGCTCCAGCCTAC |
| | | AAGCAGGGGCAGAACCAGCTCTACAACGAACTCAATCTTGGTCGGAGA |
| | | GAGGAGTACGACGTGCTGGACAAGCGGAGAGGACGGGACCCAGAAATG |
| | | GGCGGGAAGCCGCGCAGAAAGAATCCCCAAGAGGGCCTGTACAACGAG |
| | | CTCCAAAAGGATAAGATGGCAGAAGCCTATAGCGAGATTGGTATGAAA |
| | | GGGGAACGCAGAAGAGGCAAAGGCCACGACGGACTGTACCAGGGACTC |
| | | AGCACCGCCACCAAGGACACCTATGACGCTCTTCACATGCAGGCCCTG |
| | | CCGCCTCGG |

139103

| | | |
|---|---|---|
| 139103- aa | 300 | QVQLVESGGGLVQPGRSLRLSCAASGFTFSNYAMSWVRQAPGKGLGWV |
| ScFv | | SGISRSGENTYYADSVKGRFTISRDNSKNTLYLQMNSLRDEDTAVYYC |
| domain | | ARSPAHYYGGMDVWGQGTTVTVSSASGGGGSGGRASGGGGSDIVLTQS |
| | | PGTLSLSPGERATLSCRASQSISSSFLAWYQQKPGQAPRLLIYGASRR |
| | | ATGIPDRFSGSGSGTDFTLTISRLEPEDSAVYYCQQYHSSPSWTFGQG |
| | | TKLEIK |
| 139103- nt | 301 | CAAGTGCAACTCGTGGAATCTGGTGGAGGACTCGTGCAACCCGGAAGA |
| ScFv | | TCGCTTAGACTGTCGTGTGCCGCCAGCGGGTTCACTTTCTCGAACTAC |
| domain | | GCGATGTCCTGGGTCCGCCAGGCACCCGGAAAGGGACTCGGTTGGGTG |
| | | TCCGGCATTTCCCGGTCCGGCGAAAATACCTACTACGCCGACTCCGTG |
| | | AAGGGCCGCTTCACCATCTCAAGGGACAACAGCAAAAACACCCTGTAC |
| | | TTGCAAATGAACTCCCTGCGGGATGAAGATACAGCCGTGTACTATTGC |
| | | GCCCGGTCGCCTGCCCATTACTACGGCGGAATGGACGTCTGGGGACAG |
| | | GGAACCACTGTGACTGTCAGCAGCGCGTCGGGTGGCGGCGGCTCAGGG |
| | | GGTCGGGCCTCCGGGGGGGGAGGGTCCGACATCGTGCTGACCCAGTCC |
| | | CCGGGAACCCTGAGCCTGAGCCCGGGAGAGCGCGCGACCCTGTCATGC |
| | | CGGGCATCCCAGAGCATTAGCTCCTCCTTTCTCGCCTGGTATCAGCAG |
| | | AAGCCCGGACAGGCCCCGAGGCTGCTGATCTACGGCGCTAGCAGAAGG |
| | | GCTACCGGAATCCCAGACCGGTTCTCCGGCTCCGGTTCCGGGACCGAT |
| | | TTCACCCTTACTATCTCGCGCCTGGAACCTGAGGACTCCGCCGTCTAC |
| | | TACTGCCAGCAGTACCACTCATCCCCGTCGTGGACGTTCGGACAGGGC |
| | | ACCAAGCTGGAGATTAAG |
| 139103- aa | 302 | QVQLVESGGGLVQPGRSLRLSCAASGFTFSNYAMSWVRQAPGKGLGWV |
| VH | | SGISRSGENTYYADSVKGRFTISRDNSKNTLYLQMNSLRDEDTAVYYC |
| | | ARSPAHYYGGMDVWGQGTTVTVSS |
| 139103- aa | 303 | DIVLTQSPGTLSLSPGERATLSCRASQSISSSFLAWYQQKPGQAPRLL |
| VL | | IYGASRRATGIPDRFSGSGSGTDFTLTISRLEPEDSAVYYCQQYHSSP |
| | | SWTFGQGTKLEIK |
| 139103- aa | 304 | MALPVTALLLPLALLLHAARPQVQLVESGGGLVQPGRSLRLSCAASGF |
| Full CAR | | TFSNYAMSWVRQAPGKGLGWVSGISRSGENTYYADSVKGRFTISRDNS |
| | | KNTLYLQMNSLRDEDTAVYYCARSPAHYYGGMDVWGQGTTVTVSSASG |
| | | GGGSGGRASGGGGSDIVLTQSPGTLSLSPGERATLSCRASQSISSSFL |
| | | AWYQQKPGQAPRLLIYGASRRATGIPDRFSGSGSGTDFTLTISRLEPE |
| | | DSAVYYCQQYHSSPSWTFGQGTKLEIKTTTPAPRPPTPAPTIASQPLS |
| | | LRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSR |
| | | SADAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDTYD |
| | | ALHMQALPPR |
| 139103- nt | 305 | ATGGCCCTCCCTGTCACCGCCCTGCTGCTTCCGCTGGCTCTTCTGCTC |
| Full CAR | | CACGCCGCTCGGCCCCAAGTGCAACTCGTGGAATCTGGTGGAGGACTC |
| | | GTGCAACCCGGAAGATCGCTTAGACTGTCGTGTGCCGCCAGCGGGTTC |
| | | ACTTTCTCGAACTACGCGATGTCCTGGGTCCGCCAGGCACCCGGAAAG |
| | | GGACTCGGTTGGGTGTCCGGCATTTCCCGGTCCGGCGAAAATACCTAC |
| | | TACGCCGACTCCGTGAAGGGCCGCTTCACCATCTCAAGGGACAACAGC |
| | | AAAAACACCCTGTACTTGCAAATGAACTCCCTGCGGGATGAAGATACA |
| | | GCCGTGTACTATTGCGCCCGGTCGCCTGCCCATTACTACGGCGGAATG |
| | | GACGTCTGGGGACAGGGAACCACTGTGACTGTCAGCAGCGCGTCGGGT |
| | | GGCGGCGGCTCAGGGGGTCGGGCCTCCGGGGGGGGAGGGTCCGACATC |
| | | GTGCTGACCCAGTCCCCGGGAACCCTGAGCCTGAGCCCGGGAGAGCGC |
| | | GCGACCCTGTCATGCCGGGCATCCCAGAGCATTAGCTCCTCCTTTCTC |
| | | GCCTGGTATCAGCAGAAGCCCGGACAGGCCCCGAGGCTGCTGATCTAC |
| | | GGCGCTAGCAGAAGGGCTACCGGAATCCCAGACCGGTTCTCCGGCTCC |
| | | GGTTCCGGGACCGATTTCACCCTTACTATCTCGCGCCTGGAACCTGAG |
| | | GACTCCGCCGTCTACTACTGCCAGCAGTACCACTCATCCCCGTCGTGG |
| | | ACGTTCGGACAGGGCACCAAGCTGGAGATTAAGACCACTACCCCAGCA |
| | | CCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACC |
| | | CGGGGTCTTGACTTCGCCTGCGATATCTACATTTGGGCCCCTCTGGCT |
| | | GGTACTTGCGGGGTCCTGCTGCTTTCACTCGTGATCACTCTTTACTGT |
| | | AAGCGCGGTCGGAAGAAGCTGCTGTACATCTTTAAGCAACCCTTCATG |
| | | AGGCCTGTGCAGACTACTCAAGAGGAGGACGGCTGTTCATGCCGGTTC |
| | | CCAGAGGAGGAGGAAGGCGGCTGCGAACTGCGCGTGAAATTCAGCCGC |
| | | AGCGCAGATGCTCCAGCCTACAAGCAGGGGCAGAACCAGCTCTACAAC |
| | | GAACTCAATCTTGGTCGGAGAGAGGAGTACGACGTGCTGGACAAGCGG |
| | | AGAGGACGGGACCCAGAAATGGGCGGGAAGCCGCGCAGAAAGAATCCC |
| | | CAAGAGGGCCTGTACAACGAGCTCCAAAAGGATAAGATGGCAGAAGCC |
| | | TATAGCGAGATTGGTATGAAAGGGGAACGCAGAAGAGGCAAAGGCCAC |
| | | GACGGACTGTACCAGGGACTCAGCACCGCCACCAAGGACACCTATGAC |
| | | GCTCTTCACATGCAGGCCCTGCCGCCTCGG |

139105

| | | |
|---|---|---|
| 139105- aa | 306 | QVQLVESGGGLVQPGRSLRLSCAASGFTFDDYAMHWVRQAPGKGLEWV |
| ScFv | | SGISWNSGSIGYADSVKGRFTISRDNAKNSLYLQMNSLRAEDTALYYC |
| domain | | SVHSFLAYWGQGTLVTVSSASGGGGSGGRASGGGGSDIVMTQTPLSLP |
| | | VTPGEPASISCRSSQSLLHSNGYNYLDWYLQKPGQSPQLLIYLGSNRA |
| | | SGVPDRFSGSGSGTDFTLKISRVEAEDVGVYYCMQALQTPYTFGQGTK |
| | | VEIK |
| 139105- nt | 307 | CAAGTGCAACTCGTCGAATCCGGTGGAGGTCTGGTCCAACCTGGTAGA |
| ScFv | | AGCCTGAGACTGTCGTGTGCGGCCAGCGGATTCACCTTTGATGACTAT |
| domain | | GCTATGCACTGGGTGCGGCAGGCCCCAGGAAAGGGCCTGGAATGGGTG |
| | | TCGGGAATTAGCTGGAACTCCGGGTCCATTGGCTACGCCGACTCCGTG |
| | | AAGGGCCGCTTCACCATCTCCCGCGACAACGCAAAGAACTCCCTGTAC |
| | | TTGCAAATGAACTCGCTCAGGGCTGAGGATACCGCGCTGTACTACTGC |
| | | TCCGTGCATTCCTTCCTGGCCTACTGGGGACAGGGAACTCTGGTCACC |
| | | GTGTCGAGCGCCTCCGGCGGCGGGGGCTCGGGTGGACGGGCCTCGGGC |
| | | GGAGGGGGGTCCGACATCGTGATGACCCAGACCCCGCTGAGCTTGCCC |
| | | GTGACTCCCGGAGAGCCTGCATCCATCTCCTGCCGGTCATCCCAGTCC |
| | | CTTCTCCACTCCAACGGATACAACTACCTCGACTGGTACCTCCAGAAG |
| | | CCGGGACAGAGCCCTCAGCTTCTGATCTACCTGGGGTCAAATAGAGCC |
| | | TCAGGAGTGCCGGATCGGTTCAGCGGATCTGGTTCGGGAACTGATTTC |
| | | ACTCTGAAGATTTCCCGCGTGGAAGCCGAGGACGTGGGCGTCTACTAC |
| | | TGTATGCAGGCGCTGCAGACCCCCTATACCTTCGGCCAAGGGACGAAA |
| | | GTGGAGATCAAG |
| 139105- aa | 308 | QVQLVESGGGLVQPGRSLRLSCAASGFTFDDYAMHWVRQAPGKGLEWV |
| VH | | SGISWNSGSIGYADSVKGRFTISRDNAKNSLYLQMNSLRAEDTALYYC |
| | | SVHSFLAYWGQGTLVTVSS |
| 139105- aa | 309 | DIVMTQTPLSLPVTPGEPASISCRSSQSLLHSNGYNYLDWYLQKPGQS |
| VL | | PQLLIYLGSNRASGVPDRFSGSGSGTDFTLKISRVEAEDVGVYYCMQA |
| | | LQTPYTFGQGTKVEIK |
| 139105- aa | 310 | MALPVTALLLPLALLLHAARPQVQLVESGGGLVQPGRSLRLSCAASGF |
| Full CAR | | TFDDYAMHWVRQAPGKGLEWVSGISWNSGSIGYADSVKGRFTISRDNA |
| | | KNSLYLQMNSLRAEDTALYYCSVHSFLAYWGQGTLVTVSSASGGGGSG |
| | | GRASGGGGSDIVMTQTPLSLPVTPGEPASISCRSSQSLLHSNGYNYLD |
| | | WYLQKPGQSPQLLIYLGSNRASGVPDRFSGSGSGTDFTLKISRVEAED |
| | | VGVYYCMQALQTPYTFGQGTKVEIKTTTPAPRPPTPAPTIASQPLSLR |
| | | PEACRPAAGGAVHTRGLDFACDIYIWAPLAGTCGVLLLSLVITLYCKR |
| | | GRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYDAL |
| | | HMQALPPR |
| 139105- nt | 311 | ATGGCCCTCCCTGTCACCGCCCTGCTGCTTCCGCTGGCTCTTCTGCTC |
| Full CAR | | CACGCCGCTCGGCCCCAAGTGCAACTCGTCGAATCCGGTGGAGGTCTG |
| | | GTCCAACCTGGTAGAAGCCTGAGACTGTCGTGTGCGGCCAGCGGATTC |
| | | ACCTTTGATGACTATGCTATGCACTGGGTGCGGCAGGCCCCAGGAAAG |
| | | GGCCTGGAATGGGTGTCGGGAATTAGCTGGAACTCCGGGTCCATTGGC |
| | | TACGCCGACTCCGTGAAGGGCCGCTTCACCATCTCCCGCGACAACGCA |
| | | AAGAACTCCCTGTACTTGCAAATGAACTCGCTCAGGGCTGAGGATACC |
| | | GCGCTGTACTACTGCTCCGTGCATTCCTTCCTGGCCTACTGGGGACAG |
| | | GGAACTCTGGTCACCGTGTCGAGCGCCTCCGGCGGCGGGGGCTCGGGT |
| | | GGACGGGCCTCGGGCGGAGGGGGGTCCGACATCGTGATGACCCAGACC |
| | | CCGCTGAGCTTGCCCGTGACTCCCGGAGAGCCTGCATCCATCTCCTGC |
| | | CGGTCATCCCAGTCCCTTCTCCACTCCAACGGATACAACTACCTCGAC |
| | | TGGTACCTCCAGAAGCCGGGACAGAGCCCTCAGCTTCTGATCTACCTG |
| | | GGGTCAAATAGAGCCTCAGGAGTGCCGGATCGGTTCAGCGGATCTGGT |
| | | TCGGGAACTGATTTCACTCTGAAGATTTCCCGCGTGGAAGCCGAGGAC |
| | | GTGGGCGTCTACTACTGTATGCAGGCGCTGCAGACCCCCTATACCTTC |
| | | GGCCAAGGGACGAAAGTGGAGATCAAGACCACTACCCCAGCACCGAGG |
| | | CCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGT |
| | | CTTGACTTCGCCTGCGATATCTACATTTGGGCCCCTCTGGCTGGTACT |
| | | TGCGGGGTCCTGCTGCTTTCACTCGTGATCACTCTTTACTGTAAGCGC |
| | | GGTCGGAAGAAGCTGCTGTACATCTTTAAGCAACCCTTCATGAGGCCT |
| | | GTGCAGACTACTCAAGAGGAGGACGGCTGTTCATGCCGGTTCCCAGAG |
| | | GAGGAGGAAGGCGGCTGCGAACTGCGCGTGAAATTCAGCCGCAGCGCA |
| | | GATGCTCCAGCCTACAAGCAGGGGCAGAACCAGCTCTACAACGAACTC |
| | | AATCTTGGTCGGAGAGAGGAGTACGACGTGCTGGACAAGCGGAGAGGA |
| | | CGGGACCCAGAAATGGGCGGGAAGCCGCGCAGAAAGAATCCCCAAGAG |
| | | GGCCTGTACAACGAGCTCCAAAAGGATAAGATGGCAGAAGCCTATAGC |
| | | GAGATTGGTATGAAAGGGGAACGCAGAAGAGGCAAAGGCCACGACGGA |
| | | CTGTACCAGGGACTCAGCACCGCCACCAAGGACACCTATGACGCTCTT |
| | | CACATGCAGGCCCTGCCGCCTCGG |

139111

| | | |
|---|---|---|
| 139111- aa | 312 | EVQLLESGGGLVQPGGSLRLSCAVSGFALSNHGMSWVRRAPGKGLEWV |
| ScFv | | SGIVYSGSTYYAASVKGRFTISRDNSRNTLYLQMNSLRPEDTAIYYCS |
| domain | | AHGGESDVWGQGTTVTVSSASGGGGSGGRASGGGGSDIVMTQTPLSLS |
| | | VTPGQPASISCKSSQSLLRNDGKTPLYWYLQKAGQPPQLLIYEVSNRF |
| | | SGVPDRFSGSGSGTDFTLKISRVEAEDVGAYYCMQNIQFPSFGGGTKL |
| | | EIK |
| 139111- nt | 313 | GAAGTGCAATTGTTGGAATCTGGAGGAGGACTTGTGCAGCCTGGAGGA |
| ScFv | | TCACTGAGACTTTCGTGTGCGGTGTCAGGCTTCGCCCTGAGCAACCAC |
| domain | | GGCATGAGCTGGGTGCGGAGAGCCCCGGGGAAGGGTCTGGAATGGGTG |
| | | TCCGGGATCGTCTACTCCGGTTCAACTTACTACGCCGCAAGCGTGAAG |
| | | GGTCGCTTCACCATTTCCCGCGATAACTCCCGGAACACCCTGTACCTC |
| | | CAAATGAACTCCCTGCGGCCCGAGGACACCGCCATCTACTACTGTTCC |
| | | GCGCATGGAGGAGAGTCCGATGTCTGGGGACAGGGCACTACCGTGACC |
| | | GTGTCGAGCGCCTCGGGGGGAGGAGGCTCCGGCGGTCGCGCCTCCGGG |
| | | GGGGGTGGCAGCGACATTGTGATGACGCAGACTCCACTCTCGCTGTCC |
| | | GTGACCCCGGGACAGCCCGCGTCCATCTCGTGCAAGAGCTCCCAGAGC |
| | | CTGCTGAGGAACGACGGAAAGACTCCTCTGTATTGGTACCTCCAGAAG |
| | | GCTGGACAGCCCCCGCAACTGCTCATCTACGAAGTGTCAAATCGCTTC |
| | | TCCGGGGTGCCGGATCGGTTTTCCGGCTCGGGATCGGGCACCGACTTC |
| | | ACCCTGAAAATCTCCAGGGTCGAGGCCGAGGACGTGGGAGCCTACTAC |
| | | TGCATGCAAAACATCCAGTTCCCTTCCTTCGGCGGCGGCACAAAGCTG |
| | | GAGATTAAG |
| 139111- aa | 314 | EVQLLESGGGLVQPGGSLRLSCAVSGFALSNHGMSWVRRAPGKGLEWV |
| VH | | SGIVYSGSTYYAASVKGRFTISRDNSRNTLYLQMNSLRPEDTAIYYCS |
| | | AHGGESDVWGQGTTVTVSS |
| 139111- aa | 315 | DIVMTQTPLSLSVTPGQPASISCKSSQSLLRNDGKTPLYWYLQKAGQP |
| VL | | PQLLIYEVSNRFSGVPDRFSGSGSGTDFTLKISRVEAEDVGAYYCMQN |
| | | IQFPSFGGGTKLEIK |
| 139111- aa | 316 | MALPVTALLLPLALLLHAARPEVQLLESGGGLVQPGGSLRLSCAVSGF |
| Full CAR | | ALSNHGMSWVRRAPGKGLEWVSGIVYSGSTYYAASVKGRFTISRDNSR |
| | | NTLYLQMNSLRPEDTAIYYCSAHGGESDVWGQGTTVTVSSASGGGGSG |
| | | GRASGGGGSDIVMTQTPLSLSVTPGQPASISCKSSQSLLRNDGKTPLY |
| | | WYLQKAGQPPQLLIYEVSNRFSGVPDRFSGSGSGTDFTLKISRVEAED |
| | | VGAYYCMQNIQFPSFGGGTKLEIKTTTPAPRPPTPAPTIASQPLSLRP |
| | | EACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSAD |
| | | APAYKQGQNQLYNELNLGRREEYDVLDKRRGRDPEMGGKPRRKNPQEG |
| | | LYNELQKDKMAEAYSEIGMKGERRRGKGHDGLYQGLSTATKDTYDALH |
| | | MQALPPR |
| 139111- nt | 317 | ATGGCCCTCCCTGTCACCGCCCTGCTGCTTCCGCTGGCTCTTCTGCTC |
| Full CAR | | CACGCCGCTCGGCCCGAAGTGCAATTGTTGGAATCTGGAGGAGGACTT |
| | | GTGCAGCCTGGAGGATCACTGAGACTTTCGTGTGCGGTGTCAGGCTTC |
| | | GCCCTGAGCAACCACGGCATGAGCTGGGTGCGGAGAGCCCCGGGGAAG |
| | | GGTCTGGAATGGGTGTCCGGGATCGTCTACTCCGGTTCAACTTACTAC |
| | | GCCGCAAGCGTGAAGGGTCGCTTCACCATTTCCCGCGATAACTCCCGG |
| | | AACACCCTGTACCTCCAAATGAACTCCCTGCGGCCCGAGGACACCGCC |
| | | ATCTACTACTGTTCCGCGCATGGAGGAGAGTCCGATGTCTGGGGACAG |
| | | GGCACTACCGTGACCGTGTCGAGCGCCTCGGGGGGAGGAGGCTCCGGC |
| | | GGTCGCGCCTCCGGGGGGGGTGGCAGCGACATTGTGATGACGCAGACT |
| | | CCACTCTCGCTGTCCGTGACCCCGGGACAGCCCGCGTCCATCTCGTGC |
| | | AAGAGCTCCCAGAGCCTGCTGAGGAACGACGGAAAGACTCCTCTGTAT |
| | | TGGTACCTCCAGAAGGCTGGACAGCCCCCGCAACTGCTCATCTACGAA |
| | | GTGTCAAATCGCTTCTCCGGGGTGCCGGATCGGTTTTCCGGCTCGGGA |
| | | TCGGGCACCGACTTCACCCTGAAAATCTCCAGGGTCGAGGCCGAGGAC |
| | | GTGGGAGCCTACTACTGCATGCAAAACATCCAGTTCCCTTCCTTCGGC |
| | | GGCGGCACAAAGCTGGAGATTAAGACCACTACCCCAGCACCGAGGCCA |
| | | CCCACCCCGGCTCCTACCATCGCCTCCCAGCCTCTGTCCCTGCGTCCG |
| | | GAGGCATGTAGACCCGCAGCTGGTGGGGCCGTGCATACCCGGGGTCTT |
| | | GACTTCGCCTGCGATATCTACATTTGGGCCCCTCTGGCTGGTACTTGC |
| | | GGGGTCCTGCTGCTTTCACTCGTGATCACTCTTTACTGTAAGCGCGGT |
| | | CGGAAGAAGCTGCTGTACATCTTTAAGCAACCCTTCATGAGGCCTGIG |
| | | CAGACTACTCAAGAGGAGGACGGCTGTTCATGCCGGTTCCCAGAGGAG |
| | | GAGGAAGGCGGCTGCGAACTGCGCGTGAAATTCAGCCGCAGCGCAGAT |
| | | GCTCCAGCCTACAAGCAGGGGCAGAACCAGCTCTACAACGAACTCAAT |
| | | CTTGGTCGGAGAGAGGAGTACGACGTGCTGGACAAGCGGAGAGGACGG |
| | | GACCCAGAAATGGGCGGGAAGCCGCGCAGAAAGAATCCCCAAGAGGGC |
| | | CTGTACAACGAGCTCCAAAAGGATAAGATGGCAGAAGCCTATAGCGAG |
| | | ATTGGTATGAAAGGGGAACGCAGAAGAGGCAAAGGCCACGACGGACTG |
| | | TACCAGGGACTCAGCACCGCCACCAAGGACACCTATGACGCTCTTCAC |
| | | ATGCAGGCCCTGCCGCCTCGG |

139100

| | | |
|---|---|---|
| 139100- aa | 318 | QVQLVQSGAEVRKTGASVKVSCKASGYIFDNFGINWVRQAPGQGLEWM |
| ScFv | | GWINPKNNNTNYAQKFQGRVTITADESTNTAYMEVSSLRSEDTAVYYC |
| domain | | ARGPYYYQSYMDVWGQGTMVTVSSASGGGGSGGRASGGGGSDIVMTQT |
| | | PLSLPVTPGEPASISCRSSQSLLHSNGYNYLNWYLQKPGQSPQLLIYL |
| | | GSKRASGVPDRFSGSGSGTDFTLHITRVGAEDVGVYYCMQALQTPYTF |
| | | GQGTKLEIK |
| 139100- nt | 319 | CAAGTCCAACTCGTCCAGTCCGGCGCAGAAGTCAGAAAAACCGGTGCT |
| ScFv | | AGCGTGAAAGTGTCCTGCAAGGCCTCCGGCTACATTTTCGATAACTTC |
| domain | | GGAATCAACTGGGTCAGACAGGCCCCGGGCCAGGGGCTGGAATGGATG |
| | | GGATGGATCAACCCCAAGAACAACAACACCAACTACGCACAGAAGTTC |
| | | CAGGGCCGCGTGACTATCACCGCCGATGAATCGACCAATACCGCCTAC |
| | | ATGGAGGTGTCCTCCCTGCGGTCGGAGGACACTGCCGTGTATTACTGC |
| | | GCGAGGGGCCCATACTACTACCAAAGCTACATGGACGTCTGGGGACAG |
| | | GGAACCATGGTGACCGTGTCATCCGCCTCCGGTGGTGGAGGCTCCGGG |
| | | GGGCGGGCTTCAGGAGGCGGAGGAAGCGATATTGTGATGACCCAGACT |
| | | CCGCTTAGCCTGCCCGTGACTCCTGGAGAACCGGCCTCCATTTCCTGC |
| | | CGGTCCTCGCAATCACTCCTGCATTCCAACGGTTACAACTACCTGAAT |
| | | TGGTACCTCCAGAAGCCTGGCCAGTCGCCCCAGTTGCTGATCTATCTG |
| | | GGCTCGAAGCGCGCCTCCGGGGTGCCTGACCGGTTTAGCGGATCTGGG |
| | | AGCGGCACGGACTTCACTCTCCACATCACCCGCGTGGGAGCGGAGGAC |
| | | GTGGGAGTGTACTACTGTATGCAGGCGCTGCAGACTCCGTACACATTC |
| | | GGACAGGGCACCAAGCTGGAGATCAAG |
| 139100- aa | 320 | QVQLVQSGAEVRKTGASVKVSCKASGYIFDNFGINWVRQAPGQGLEWM |
| VH | | GWINPKNNNTNYAQKFQGRVTITADESTNTAYMEVSSLRSEDTAVYYC |
| | | ARGPYYYQSYMDVWGQGTMVTVSS |
| 139100- aa | 321 | DIVMTQTPLSLPVTPGEPASISCRSSQSLLHSNGYNYLNWYLQKPGQS |
| VL | | PQLLIYLGSKRASGVPDRFSGSGSGTDFTLHITRVGAEDVGVYYCMQA |
| | | LQTPYTFGQGTKLEIK |
| 139100- aa | 322 | MALPVTALLLPLALLLHAARPQVQLVQSGAEVRKTGASVKVSCKASGY |
| Full CAR | | IFDNFGINWVRQAPGQGLEWMGWINPKNNNTNYAQKFQGRVTITADES |
| | | TNTAYMEVSSLRSEDTAVYYCARGPYYYQSYMDVWGQGTMVTVSSASG |
| | | GGGSGGRASGGGGSDIVMTQTPLSLPVTPGEPASISCRSSQSLLHSNG |
| | | YNYLNWYLQKPGQSPQLLIYLGSKRASGVPDRFSGSGSGTDFTLHITR |
| | | VGAEDVGVYYCMQALQTPYTFGQGTKLEIKTTTPAPRPPTPAPTIASQ |
| | | PLSLRPEACRPAAGGAVHTRGLDFACDIYIWAPLAGTCGVLLLSLVIT |
| | | LYCKRGRKKLLYIFKQPFMRPVQTTQEEDGCSCRFPEEEEGGCELRVK |
| | | FSRSADAPAYKQGQNQLYNELNLGRREEYDVLDKRRGRDPEMGGKPRR |
| | | KNPQEGLYNELQKDKMAEAYSEIGMKGERRRGKGHDGLYQGLSTATKD |
| | | TYDALHMQALPPR |
| 139100- nt | 323 | ATGGCCCTCCCTGTCACCGCCCTGCTGCTTCCGCTGGCTCTTCTGCTC |
| Full CAR | | CACGCCGCTCGGCCCCAAGTCCAACTCGTCCAGTCCGGCGCAGAAGTC |
| | | AGAAAAACCGGTGCTAGCGTGAAAGTGTCCTGCAAGGCCTCCGGCTAC |
| | | ATTTTCGATAACTTCGGAATCAACTGGGTCAGACAGGCCCCGGGCCAG |
| | | GGGCTGGAATGGATGGGATGGATCAACCCCAAGAACAACAACACCAAC |
| | | TACGCACAGAAGTTCCAGGGCCGCGTGACTATCACCGCCGATGAATCG |
| | | ACCAATACCGCCTACATGGAGGTGTCCTCCCTGCGGTCGGAGGACACT |
| | | GCCGTGTATTACTGCGCGAGGGGCCCATACTACTACCAAAGCTACATG |
| | | GACGTCTGGGGACAGGGAACCATGGTGACCGTGTCATCCGCCTCCGGT |
| | | GGTGGAGGCTCCGGGGGGCGGGCTTCAGGAGGCGGAGGAAGCGATATT |
| | | GTGATGACCCAGACTCCGCTTAGCCTGCCCGTGACTCCTGGAGAACCG |
| | | GCCTCCATTTCCTGCCGGTCCTCGCAATCACTCCTGCATTCCAACGGT |
| | | TACAACTACCTGAATTGGTACCTCCAGAAGCCTGGCCAGTCGCCCCAG |
| | | TTGCTGATCTATCTGGGCTCGAAGCGCGCCTCCGGGGTGCCTGACCGG |
| | | TTTAGCGGATCTGGGAGCGGCACGGACTTCACTCTCCACATCACCCGC |
| | | GTGGGAGCGGAGGACGTGGGAGTGTACTACTGTATGCAGGCGCTGCAG |
| | | ACTCCGTACACATTCGGACAGGGCACCAAGCTGGAGATCAAGACCACT |
| | | ACCCCAGCACCGAGGCCACCCACCCCGGCTCCTACCATCGCCTCCCAG |
| | | CCTCTGTCCCTGCGTCCGGAGGCATGTAGACCCGCAGCTGGTGGGGCC |
| | | GTGCATACCCGGGGTCTTGACTTCGCCTGCGATATCTACATTTGGGCC |
| | | CCTCTGGCTGGTACTTGCGGGGTCCTGCTGCTTTCACTCGTGATCACT |
| | | CTTTACTGTAAGCGCGGTCGGAAGAAGCTGCTGTACATCTTTAAGCAA |
| | | CCCTTCATGAGGCCTGTGCAGACTACTCAAGAGGAGGACGGCTGTTCA |
| | | TGCCGGTTCCCAGAGGAGGAGGAAGGCGGCTGCGAACTGCGCGTGAAA |
| | | TTCAGCCGCAGCGCAGATGCTCCAGCCTACAAGCAGGGGCAGAACCAG |
| | | CTCTACAACGAACTCAATCTTGGTCGGAGAGAGGAGTACGACGTGCTG |
| | | GACAAGCGGAGAGGACGGGACCCAGAAATGGGCGGGAAGCCGCGCAGA |
| | | AAGAATCCCCAAGAGGGCCTGTACAACGAGCTCCAAAAGGATAAGATG |
| | | GCAGAAGCCTATAGCGAGATTGGTATGAAAGGGGAACGCAGAAGAGGC |
| | | AAAGGCCACGACGGACTGTACCAGGGACTCAGCACCGCCACCAAGGAC |
| | | ACCTATGACGCTCTTCACATGCAGGCCCTGCCGCCTCGG |

139101

| | | |
|---|---|---|
| 139101- aa | 324 | QVQLQESGGGLVQPGGSLRLSCAASGFTFSSDAMTWVRQAPGKGLEWV |
| ScFv | | SVISGSGGTTYYADSVKGRFTISRDNSKNTLYLQMNSLRAEDTAVYYC |
| domain | | AKLDSSGYYYARGPRYWGQGTLVTVSSASGGGGSGGRASGGGGSDIQL |
| | | TQSPSSLSASVGDRVTITCRASQSISSYLNWYQQKPGKAPKLLIYGAS |
| | | TLASGVPARFSGSGSGTHFTLTINSLQSEDSATYYCQQSYKRASFGQG |
| | | TKVEIK |
| 139101- nt | 325 | CAAGTGCAACTTCAAGAATCAGGCGGAGGACTCGTGCAGCCCGGAGGA |
| ScFv | | TCATTGCGGCTCTCGTGCGCCGCCTCGGGCTTCACCTTCTCGAGCGAC |
| domain | | GCCATGACCTGGGTCCGCCAGGCCCCGGGGAAGGGGCTGGAATGGGTG |
| | | TCTGTGATTTCCGGCTCCGGGGGAACTACGTACTACGCCGATTCCGTG |
| | | AAAGGTCGCTTCACTATCTCCCGGGACAACAGCAAGAACACCCTTTAT |
| | | CTGCAAATGAATTCCCTCCGCGCCGAGGACACCGCCGTGTACTACTGC |
| | | GCCAAGCTGGACTCCTCGGGCTACTACTATGCCCGGGGTCCGAGATAC |
| | | TGGGGACAGGGAACCCTCGTGACCGTGTCCTCCGCGTCCGGCGGAGGA |
| | | GGGTCGGGAGGGCGGGCCTCCGGCGGCGGCGGTTCGGACATCCAGCTG |
| | | ACCCAGTCCCCATCCTCACTGAGCGCAAGCGTGGGCGACAGAGTCACC |
| | | ATTACATGCAGGGCGTCCCAGAGCATCAGCTCCTACCTGAACTGGTAC |
| | | CAACAGAAGCCTGGAAAGGCTCCTAAGCTGTTGATCTACGGGGCTTCG |
| | | ACCCTGGCATCCGGGGTGCCCGCGAGGTTTAGCGGAAGCGGTAGCGGC |
| | | ACTCACTTCACTCTGACCATTAACAGCCTCCAGTCCGAGGATTCAGCC |
| | | ACTTACTACTGTCAGCAGTCCTACAAGCGGGCCAGCTTCGGACAGGGC |
| | | ACTAAGGTCGAGATCAAG |
| 139101- aa | 326 | QVQLQESGGGLVQPGGSLRLSCAASGFTFSSDAMTWVRQAPGKGLEWV |
| VH | | SVISGSGGTTYYADSVKGRFTISRDNSKNTLYLQMNSLRAEDTAVYYC |
| | | AKLDSSGYYYARGPRYWGQGTLVTVSS |
| 139101- aa | 327 | DIQLTQSPSSLSASVGDRVTITCRASQSISSYLNWYQQKPGKAPKLLI |
| VL | | YGASTLASGVPARFSGSGSGTHFTLTINSLQSEDSATYYCQQSYKRAS |
| | | FGQGTKVEIK |
| 139101- aa | 328 | MALPVTALLLPLALLLHAARPQVQLQESGGGLVQPGGSLRLSCAASGF |
| Full CAR | | TFSSDAMTWVRQAPGKGLEWVSVISGSGGTTYYADSVKGRFTISRDNS |
| | | KNTLYLQMNSLRAEDTAVYYCAKLDSSGYYYARGPRYWGQGTLVTVSS |
| | | ASGGGGSGGRASGGGGSDIQLTQSPSSLSASVGDRVTITCRASQSISS |
| | | YLNWYQQKPGKAPKLLIYGASTLASGVPARFSGSGSGTHFTLTINSLQ |
| | | SEDSATYYCQQSYKRASFGQGTKVEIKTTTPAPRPPTPAPTIASQPLS |
| | | LRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSR |
| | | SADAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDTYD |
| | | ALHMQALPPR |
| 139101- nt | 329 | ATGGCCCTCCCTGTCACCGCCCTGCTGCTTCCGCTGGCTCTTCTGCTC |
| Full CAR | | CACGCCGCTCGGCCCCAAGTGCAACTTCAAGAATCAGGCGGAGGACTC |
| | | GTGCAGCCCGGAGGATCATTGCGGCTCTCGTGCGCCGCCTCGGGCTTC |
| | | ACCTTCTCGAGCGACGCCATGACCTGGGTCCGCCAGGCCCCGGGGAAG |
| | | GGGCTGGAATGGGTGTCTGTGATTTCCGGCTCCGGGGGAACTACGTAC |
| | | TACGCCGATTCCGTGAAAGGTCGCTTCACTATCTCCCGGGACAACAGC |
| | | AAGAACACCCTTTATCTGCAAATGAATTCCCTCCGCGCCGAGGACACC |
| | | GCCGTGTACTACTGCGCCAAGCTGGACTCCTCGGGCTACTACTATGCC |
| | | CGGGGTCCGAGATACTGGGGACAGGGAACCCTCGTGACCGTGTCCTCC |
| | | GCGTCCGGCGGAGGAGGGTCGGGAGGGCGGGCCTCCGGCGGCGGCGGT |
| | | TCGGACATCCAGCTGACCCAGTCCCCATCCTCACTGAGCGCAAGCGTG |
| | | GGCGACAGAGTCACCATTACATGCAGGGCGTCCCAGAGCATCAGCTCC |
| | | TACCTGAACTGGTACCAACAGAAGCCTGGAAAGGCTCCTAAGCTGTTG |
| | | ATCTACGGGGCTTCGACCCTGGCATCCGGGGTGCCCGCGAGGTTTAGC |
| | | GGAAGCGGTAGCGGCACTCACTTCACTCTGACCATTAACAGCCTCCAG |
| | | TCCGAGGATTCAGCCACTTACTACTGTCAGCAGTCCTACAAGCGGGCC |
| | | AGCTTCGGACAGGGCACTAAGGTCGAGATCAAGACCACTACCCCAGCA |
| | | CCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACC |
| | | CGGGGTCTTGACTTCGCCTGCGATATCTACATTTGGGCCCCTCTGGCT |
| | | GGTACTTGCGGGGTCCTGCTGCTTTCACTCGTGATCACTCTTTACTGT |
| | | AAGCGCGGTCGGAAGAAGCTGCTGTACATCTTTAAGCAACCCTTCATG |
| | | AGGCCTGTGCAGACTACTCAAGAGGAGGACGGCTGTTCATGCCGGTTC |
| | | CCAGAGGAGGAGGAAGGCGGCTGCGAACTGCGCGTGAAATTCAGCCGC |
| | | AGCGCAGATGCTCCAGCCTACAAGCAGGGGCAGAACCAGCTCTACAAC |
| | | GAACTCAATCTTGGTCGGAGAGAGGAGTACGACGTGCTGGACAAGCGG |
| | | AGAGGACGGGACCCAGAAATGGGCGGGAAGCCGCGCAGAAAGAATCCC |
| | | CAAGAGGGCCTGTACAACGAGCTCCAAAAGGATAAGATGGCAGAAGCC |
| | | TATAGCGAGATTGGTATGAAAGGGGAACGCAGAAGAGGCAAAGGCCAC |
| | | GACGGACTGTACCAGGGACTCAGCACCGCCACCAAGGACACCTATGAC |
| | | GCTCTTCACATGCAGGCCCTGCCGCCTCGG |

139102

| | | |
|---|---|---|
| 139102- aa | 330 | QVQLVQSGAEVKKPGASVKVSCKASGYTFSNYGITWVRQAPGQGLEWM |
| ScFv | | GWISAYNGNTNYAQKFQGRVTMTRNTSISTAYMELSSLRSEDTAVYYC |
| domain | | ARGPYYYYMDVWGKGTMVTVSSASGGGGSGGRASGGGGSEIVMTQSPL |
| | | SLPVTPGEPASISCRSSQSLLYSNGYNYVDWYLQKPGQSPQLLIYLGS |
| | | NRASGVPDRFSGSGSGTDFKLQISRVEAEDVGIYYCMQGRQFPYSFGQ |
| | | GTKVEIK |
| 139102- nt | 331 | CAAGTCCAACTGGTCCAGAGCGGTGCAGAAGTGAAGAAGCCCGGAGCG |
| ScFv | | AGCGTGAAAGTGTCCTGCAAGGCTTCCGGGTACACCTTCTCCAACTAC |
| domain | | GGCATCACTTGGGTGCGCCAGGCCCCGGGACAGGGCCTGGAATGGATG |
| | | GGGTGGATTTCCGCGTACAACGGCAATACGAACTACGCTCAGAAGTTC |
| | | CAGGGTAGAGTGACCATGACTAGGAACACCTCCATTTCCACCGCCTAC |
| | | ATGGAACTGTCCTCCCTGCGGAGCGAGGACACCGCCGTGTACTATTGC |
| | | GCCCGGGGACCATACTACTACTACATGGATGTCTGGGGGAAGGGGACT |
| | | ATGGTCACCGTGTCATCCGCCTCGGGAGGCGGCGGATCAGGAGGACGC |
| | | GCCTCTGGTGGTGGAGGATCGGAGATCGTGATGACCCAGAGCCCTCTC |
| | | TCCTTGCCCGTGACTCCTGGGGAGCCCGCATCCATTTCATGCCGGAGC |
| | | TCCCAGTCACTTCTCTACTCCAACGGCTATAACTACGTGGATTGGTAC |
| | | CTCCAAAAGCCGGGCCAGAGCCCGCAGCTGCTGATCTACCTGGGCTCG |
| | | AACAGGGCCAGCGGAGTGCCTGACCGGTTCTCCGGGTCGGGAAGCGGG |
| | | ACCGACTTCAAGCTGCAAATCTCGAGAGTGGAGGCCGAGGACGTGGGA |
| | | ATCTACTACTGTATGCAGGGCCGCCAGTTTCCGTACTCGTTCGGACAG |
| | | GGCACCAAAGTGGAAATCAAG |
| 139102- aa | 332 | QVQLVQSGAEVKKPGASVKVSCKASGYTFSNYGITWVRQAPGQGLEWM |
| VH | | GWISAYNGNTNYAQKFQGRVTMTRNTSISTAYMELSSLRSEDTAVYYC |
| | | ARGPYYYYMDVWGKGTMVTVSS |
| 139102- aa | 333 | EIVMTQSPLSLPVTPGEPASISCRSSQSLLYSNGYNYVDWYLQKPGQS |
| VL | | PQLLIYLGSNRASGVPDRFSGSGSGTDFKLQISRVEAEDVGIYYCMQG |
| | | RQFPYSFGQGTKVEIK |
| 139102- aa | 334 | MALPVTALLLPLALLLHAARPQVQLVQSGAEVKKPGASVKVSCKASGY |
| Full CAR | | TFSNYGITWVRQAPGQGLEWMGWISAYNGNTNYAQKFQGRVTMTRNTS |
| | | ISTAYMELSSLRSEDTAVYYCARGPYYYYMDVWGKGTMVTVSSASGGG |
| | | GSGGRASGGGGSEIVMTQSPLSLPVTPGEPASISCRSSQSLLYSNGYN |
| | | YVDWYLQKPGQSPQLLIYLGSNRASGVPDRFSGSGSGTDFKLQISRVE |
| | | AEDVGIYYCMQGRQFPYSFGQGTKVEIKTTTPAPRPPTPAPTIASQPL |
| | | SLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFS |
| | | RSADAPAYKQGQNQLYNELNLGRREEYDVLDKRRGRDPEMGGKPRRKN |
| | | PQEGLYNELQKDKMAEAYSEIGMKGERRRGKGHDGLYQGLSTATKDTY |
| | | DALHMQALPPR |
| 139102- nt | 335 | ATGGCCCTCCCTGTCACCGCCCTGCTGCTTCCGCTGGCTCTTCTGCTC |
| Full CAR | | CACGCCGCTCGGCCCCAAGTCCAACTGGTCCAGAGCGGTGCAGAAGTG |
| | | AAGAAGCCCGGAGCGAGCGTGAAAGTGTCCTGCAAGGCTTCCGGGTAC |
| | | ACCTTCTCCAACTACGGCATCACTTGGGTGCGCCAGGCCCCGGGACAG |
| | | GGCCTGGAATGGATGGGGTGGATTTCCGCGTACAACGGCAATACGAAC |
| | | TACGCTCAGAAGTTCCAGGGTAGAGTGACCATGACTAGGAACACCTCC |
| | | ATTTCCACCGCCTACATGGAACTGTCCTCCCTGCGGAGCGAGGACACC |
| | | GCCGTGTACTATTGCGCCCGGGGACCATACTACTACTACATGGATGTC |
| | | TGGGGGAAGGGGACTATGGTCACCGTGTCATCCGCCTCGGGAGGCGGC |
| | | GGATCAGGAGGACGCGCCTCTGGTGGTGGAGGATCGGAGATCGTGATG |
| | | ACCCAGAGCCCTCTCTCCTTGCCCGTGACTCCTGGGGAGCCCGCATCC |
| | | ATTTCATGCCGGAGCTCCCAGTCACTTCTCTACTCCAACGGCTATAAC |
| | | TACGTGGATTGGTACCTCCAAAAGCCGGGCCAGAGCCCGCAGCTGCTG |
| | | ATCTACCTGGGCTCGAACAGGGCCAGCGGAGTGCCTGACCGGTTCTCC |
| | | GGGTCGGGAAGCGGGACCGACTTCAAGCTGCAAATCTCGAGAGTGGAG |
| | | GCCGAGGACGTGGGAATCTACTACTGTATGCAGGGCCGCCAGTTTCCG |
| | | TACTCGTTCGGACAGGGCACCAAAGTGGAAATCAAGACCACTACCCCA |
| | | GCACCGAGGCCACCCACCCCGGCTCCTACCATCGCCTCCCAGCCTCTG |
| | | TCCCTGCGTCCGGAGGCATGTAGACCCGCAGCTGGTGGGGCCGTGCAT |
| | | ACCCGGGGTCTTGACTTCGCCTGCGATATCTACATTTGGGCCCCTCTG |
| | | GCTGGTACTTGCGGGGTCCTGCTGCTTTCACTCGTGATCACTCTTTAC |
| | | TGTAAGCGCGGTCGGAAGAAGCTGCTGTACATCTTTAAGCAACCCTTC |
| | | ATGAGGCCTGTGCAGACTACTCAAGAGGAGGACGGCTGTTCATGCCGG |
| | | TTCCCAGAGGAGGAGGAAGGCGGCTGCGAACTGCGCGTGAAATTCAGC |
| | | CGCAGCGCAGATGCTCCAGCCTACAAGCAGGGGCAGAACCAGCTCTAC |
| | | AACGAACTCAATCTTGGTCGGAGAGAGGAGTACGACGTGCTGGACAAG |
| | | CGGAGAGGACGGGACCCAGAAATGGGCGGGAAGCCGCGCAGAAAGAAT |
| | | CCCCAAGAGGGCCTGTACAACGAGCTCCAAAAGGATAAGATGGCAGAA |
| | | GCCTATAGCGAGATTGGTATGAAAGGGGAACGCAGAAGAGGCAAAGGC |
| | | CACGACGGACTGTACCAGGGACTCAGCACCGCCACCAAGGACACCTAT |
| | | GACGCTCTTCACATGCAGGCCCTGCCGCCTCGG |

139104

| | | |
|---|---|---|
| 139104- aa | 336 | EVQLLETGGGLVQPGGSLRLSCAVSGFALSNHGMSWVRRAPGKGLEWV |
| ScFv | | SGIVYSGSTYYAASVKGRFTISRDNSRNTLYLQMNSLRPEDTAIYYCS |
| domain | | AHGGESDVWGQGTTVTVSSASGGGGSGGRASGGGGSEIVLTQSPATLS |
| | | VSPGESATLSCRASQSVSSNLAWYQQKPGQAPRLLIYGASTRASGIPD |
| | | RFSGSGSGTDFTLTISSLQAEDVAVYYCQQYGSSLTFGGGTKVEIK |
| 139104- nt | 337 | GAAGTGCAATTGCTCGAAACTGGAGGAGGTCTGGTGCAACCTGGAGGA |
| ScFv | | TCACTTCGCCTGTCCTGCGCCGTGTCGGGCTTTGCCCTGTCCAACCAT |
| domain | | GGAATGAGCTGGGTCCGCCGCGCGCCGGGGAAGGGCCTCGAATGGGTG |
| | | TCCGGCATCGTCTACTCCGGCTCCACCTACTACGCCGCGTCCGTGAAG |
| | | GGCCGGTTCACGATTTCACGGGACAACTCGCGGAACACCCTGTACCTC |
| | | CAAATGAATTCCCTTCGGCCGGAGGATACTGCCATCTACTACTGCTCC |
| | | GCCCACGGTGGCGAATCCGACGTCTGGGGCCAGGGAACCACCGTGACC |
| | | GTGTCCAGCGCGTCCGGGGGAGGAGGAAGCGGGGGTAGAGCATCGGGT |
| | | GGAGGCGGATCAGAGATCGTGCTGACCCAGTCCCCCGCCACCTTGAGC |
| | | GTGTCACCAGGAGAGTCCGCCACCCTGTCATGCCGCGCCAGCCAGTCC |
| | | GTGTCCTCCAACCTGGCTTGGTACCAGCAGAAGCCGGGGCAGGCCCCT |
| | | AGACTCCTGATCTATGGGGCGTCGACCCGGGCATCTGGAATTCCCGAT |
| | | AGGTTCAGCGGATCGGGCTCGGGCACTGACTTCACTCTGACCATCTCC |
| | | TCGCTGCAAGCCGAGGACGTGGCTGTGTACTACTGTCAGCAGTACGGA |
| | | AGCTCCCTGACTTTCGGTGGCGGGACCAAAGTCGAGATTAAG |
| 139104- aa | 338 | EVQLLETGGGLVQPGGSLRLSCAVSGFALSNHGMSWVRRAPGKGLEWV |
| VH | | SGIVYSGSTYYAASVKGRFTISRDNSRNTLYLQMNSLRPEDTAIYYCS |
| | | AHGGESDVWGQGTTVTVSS |
| 139104- aa | 339 | EIVLTQSPATLSVSPGESATLSCRASQSVSSNLAWYQQKPGQAPRLLI |
| VL | | YGASTRASGIPDRFSGSGSGTDFTLTISSLQAEDVAVYYCQQYGSSLT |
| | | FGGGTKVEIK |
| 139104- aa | 340 | MALPVTALLLPLALLLHAARPEVQLLETGGGLVQPGGSLRLSCAVSGF |
| Full CAR | | ALSNHGMSWVRRAPGKGLEWVSGIVYSGSTYYAASVKGRFTISRDNSR |
| | | NTLYLQMNSLRPEDTAIYYCSAHGGESDVWGQGTTVTVSSASGGGGSG |
| | | GRASGGGGSEIVLTQSPATLSVSPGESATLSCRASQSVSSNLAWYQQK |
| | | PGQAPRLLIYGASTRASGIPDRFSGSGSGTDFTLTISSLQAEDVAVYY |
| | | CQQYGSSLTFGGGTKVEIKTTTPAPRPPTPAPTIASQPLSLRPEACRP |
| | | AAGGAVHTRGLDFACDIYIWAPLAGTCGVLLLSLVITLYCKRGRKKLL |
| | | YIFKQPFMRPVQTTQEEDGCSCRFPEEEEGGCELRVKFSRSADAPAYK |
| | | QGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALP |
| | | PR |
| 139104- nt | 341 | ATGGCCCTCCCTGTCACCGCCCTGCTGCTTCCGCTGGCTCTTCTGCTC |
| Full CAR | | CACGCCGCTCGGCCCGAAGTGCAATTGCTCGAAACTGGAGGAGGTCTG |
| | | GTGCAACCTGGAGGATCACTTCGCCTGTCCTGCGCCGTGTCGGGCTTT |
| | | GCCCTGTCCAACCATGGAATGAGCTGGGTCCGCCGCGCGCCGGGGAAG |
| | | GGCCTCGAATGGGTGTCCGGCATCGTCTACTCCGGCTCCACCTACTAC |
| | | GCCGCGTCCGTGAAGGGCCGGTTCACGATTTCACGGGACAACTCGCGG |
| | | AACACCCTGTACCTCCAAATGAATTCCCTTCGGCCGGAGGATACTGCC |
| | | ATCTACTACTGCTCCGCCCACGGTGGCGAATCCGACGTCTGGGGCCAG |
| | | GGAACCACCGTGACCGTGTCCAGCGCGTCCGGGGGAGGAGGAAGCGGG |
| | | GGTAGAGCATCGGGTGGAGGCGGATCAGAGATCGTGCTGACCCAGTCC |
| | | CCCGCCACCTTGAGCGTGTCACCAGGAGAGTCCGCCACCCTGTCATGC |
| | | CGCGCCAGCCAGTCCGTGTCCTCCAACCTGGCTTGGTACCAGCAGAAG |
| | | CCGGGGCAGGCCCCTAGACTCCTGATCTATGGGGCGTCGACCCGGGCA |
| | | TCTGGAATTCCCGATAGGTTCAGCGGATCGGGCTCGGGCACTGACTTC |
| | | ACTCTGACCATCTCCTCGCTGCAAGCCGAGGACGTGGCTGTGTACTAC |
| | | TGTCAGCAGTACGGAAGCTCCCTGACTTTCGGTGGCGGGACCAAAGTC |
| | | GAGATTAAGACCACTACCCCAGCACCGAGGCCACCCACCCCGGCTCCT |
| | | ACCATCGCCTCCCAGCCTCTGTCCCTGCGTCCGGAGGCATGTAGACCC |
| | | GCAGCTGGTGGGGCCGTGCATACCCGGGGTCTTGACTTCGCCTGCGAT |
| | | ATCTACATTTGGGCCCCTCTGGCTGGTACTTGCGGGGTCCTGCTGCTT |
| | | TCACTCGTGATCACTCTTTACTGTAAGCGCGGTCGGAAGAAGCTGCTG |
| | | TACATCTTTAAGCAACCCTTCATGAGGCCTGTGCAGACTACTCAAGAG |
| | | GAGGACGGCTGTTCATGCCGGTTCCCAGAGGAGGAGGAAGGCGGCTGC |
| | | GAACTGCGCGTGAAATTCAGCCGCAGCGCAGATGCTCCAGCCTACAAG |
| | | CAGGGGCAGAACCAGCTCTACAACGAACTCAATCTTGGTCGGAGAGAG |
| | | GAGTACGACGTGCTGGACAAGCGGAGAGGACGGGACCCAGAAATGGGC |
| | | GGGAAGCCGCGCAGAAAGAATCCCCAAGAGGGCCTGTACAACGAGCTC |
| | | CAAAAGGATAAGATGGCAGAAGCCTATAGCGAGATTGGTATGAAAGGG |
| | | GAACGCAGAAGAGGCAAAGGCCACGACGGACTGTACCAGGGACTCAGC |
| | | ACCGCCACCAAGGACACCTATGACGCTCTTCACATGCAGGCCCTGCCG |
| | | CCTCGG |

139106

| | | |
|---|---|---|
| 139106- aa | 342 | EVQLVETGGGLVQPGGSLRLSCAVSGFALSNHGMSWVRRAPGKGLEWV |
| ScFv | | SGIVYSGSTYYAASVKGRFTISRDNSRNTLYLQMNSLRPEDTAIYYCS |
| domain | | AHGGESDVWGQGTTVTVSSASGGGGSGGRASGGGGSEIVMTQSPATLS |
| | | VSPGERATLSCRASQSVSSKLAWYQQKPGQAPRLLMYGASIRATGIPD |
| | | RFSGSGSGTEFTLTISSLEPEDFAVYYCQQYGSSSWTFGQGTKVEIK |
| 139106- nt | 343 | GAAGTGCAATTGGTGGAAACTGGAGGAGGACTTGTGCAACCTGGAGGA |
| ScFv | | TCATTGAGACTGAGCTGCGCAGTGTCGGGATTCGCCCTGAGCAACCAT |
| domain | | GGAATGTCCTGGGTCAGAAGGGCCCCTGGAAAAGGCCTCGAATGGGTG |
| | | TCAGGGATCGTGTACTCCGGTTCCACTTACTACGCCGCCTCCGTGAAG |
| | | GGGCGCTTCACTATCTCACGGGATAACTCCCGCAATACCCTGTACCTC |
| | | CAAATGAACAGCCTGCGGCCGGAGGATACCGCCATCTACTACTGTTCC |
| | | GCCCACGGTGGAGAGTCTGACGTCTGGGGCCAGGGAACTACCGTGACC |
| | | GTGTCCTCCGCGTCCGGCGGTGGAGGGAGCGGCGGCCGCGCCAGCGGC |
| | | GGCGGAGGCTCCGAGATCGTGATGACCCAGAGCCCCGCTACTCTGTCG |
| | | GTGTCGCCCGGAGAAAGGGCGACCCTGTCCTGCCGGGCGTCGCAGTCC |
| | | GTGAGCAGCAAGCTGGCTTGGTACCAGCAGAAGCCGGGCCAGGCACCA |
| | | CGCCTGCTTATGTACGGTGCCTCCATTCGGGCCACCGGAATCCCGGAC |
| | | CGGTTCTCGGGGTCGGGGTCCGGTACCGAGTTCACACTGACCATTTCC |
| | | TCGCTCGAGCCCGAGGACTTTGCCGTCTATTACTGCCAGCAGTACGGC |
| | | TCCTCCTCATGGACGTTCGGCCAGGGGACCAAGGTCGAAATCAAG |
| 139106- aa | 344 | EVQLVETGGGLVQPGGSLRLSCAVSGFALSNHGMSWVRRAPGKGLEWV |
| VH | | SGIVYSGSTYYAASVKGRFTISRDNSRNTLYLQMNSLRPEDTAIYYCS |
| | | AHGGESDVWGQGTTVTVSS |
| 139106- aa | 345 | EIVMTQSPATLSVSPGERATLSCRASQSVSSKLAWYQQKPGQAPRLLM |
| VL | | YGASIRATGIPDRFSGSGSGTEFTLTISSLEPEDFAVYYCQQYGSSSW |
| | | TFGQGTKVEIK |
| 139106- aa | 346 | MALPVTALLLPLALLLHAARPEVQLVETGGGLVQPGGSLRLSCAVSGF |
| Full CAR | | ALSNHGMSWVRRAPGKGLEWVSGIVYSGSTYYAASVKGRFTISRDNSR |
| | | NTLYLQMNSLRPEDTAIYYCSAHGGESDVWGQGTTVTVSSASGGGGSG |
| | | GRASGGGGSEIVMTQSPATLSVSPGERATLSCRASQSVSSKLAWYQQK |
| | | PGQAPRLLMYGASIRATGIPDRFSGSGSGTEFTLTISSLEPEDFAVYY |
| | | CQQYGSSSWTFGQGTKVEIKTTTPAPRPPTPAPTIASQPLSLRPEACR |
| | | PAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQAL |
| | | PPR |
| 139106- nt | 347 | ATGGCCCTCCCTGTCACCGCCCTGCTGCTTCCGCTGGCTCTTCTGCTC |
| Full CAR | | CACGCCGCTCGGCCCGAAGTGCAATTGGTGGAAACTGGAGGAGGACTT |
| | | GTGCAACCTGGAGGATCATTGAGACTGAGCTGCGCAGTGTCGGGATTC |
| | | GCCCTGAGCAACCATGGAATGTCCTGGGTCAGAAGGGCCCCTGGAAAA |
| | | GGCCTCGAATGGGTGTCAGGGATCGTGTACTCCGGTTCCACTTACTAC |
| | | GCCGCCTCCGTGAAGGGGCGCTTCACTATCTCACGGGATAACTCCCGC |
| | | AATACCCTGTACCTCCAAATGAACAGCCTGCGGCCGGAGGATACCGCC |
| | | ATCTACTACTGTTCCGCCCACGGTGGAGAGTCTGACGTCTGGGGCCAG |
| | | GGAACTACCGTGACCGTGTCCTCCGCGTCCGGCGGTGGAGGGAGCGGC |
| | | GGCCGCGCCAGCGGCGGCGGAGGCTCCGAGATCGTGATGACCCAGAGC |
| | | CCCGCTACTCTGTCGGTGTCGCCCGGAGAAAGGGCGACCCTGTCCTGC |
| | | CGGGCGTCGCAGTCCGTGAGCAGCAAGCTGGCTTGGTACCAGCAGAAG |
| | | CCGGGCCAGGCACCACGCCTGCTTATGTACGGTGCCTCCATTCGGGCC |
| | | ACCGGAATCCCGGACCGGTTCTCGGGGTCGGGGTCCGGTACCGAGTTC |
| | | ACACTGACCATTTCCTCGCTCGAGCCCGAGGACTTTGCCGTCTATTAC |
| | | TGCCAGCAGTACGGCTCCTCCTCATGGACGTTCGGCCAGGGGACCAAG |
| | | GTCGAAATCAAGACCACTACCCCAGCACCGAGGCCACCCACCCCGGCT |
| | | CCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGC |
| | | GATATCTACATTTGGGCCCCTCTGGCTGGTACTTGCGGGGTCCTGCTG |
| | | CTTTCACTCGTGATCACTCTTTACTGTAAGCGCGGTCGGAAGAAGCTG |
| | | CTGTACATCTTTAAGCAACCCTTCATGAGGCCTGTGCAGACTACTCAA |
| | | GAGGAGGACGGCTGTTCATGCCGGTTCCCAGAGGAGGAGGAAGGCGGC |
| | | TGCGAACTGCGCGTGAAATTCAGCCGCAGCGCAGATGCTCCAGCCTAC |
| | | AAGCAGGGGCAGAACCAGCTCTACAACGAACTCAATCTTGGTCGGAGA |
| | | GAGGAGTACGACGTGCTGGACAAGCGGAGAGGACGGGACCCAGAAATG |
| | | GGCGGGAAGCCGCGCAGAAAGAATCCCCAAGAGGGCCTGTACAACGAG |
| | | CTCCAAAAGGATAAGATGGCAGAAGCCTATAGCGAGATTGGTATGAAA |
| | | GGGGAACGCAGAAGAGGCAAAGGCCACGACGGACTGTACCAGGGACTC |
| | | AGCACCGCCACCAAGGACACCTATGACGCTCTTCACATGCAGGCCCTG |
| | | CCGCCTCGG |

139107

| | | |
|---|---|---|
| 139107- aa | 348 | EVQLVETGGGVVQPGGSLRLSCAVSGFALSNHGMSWVRRAPGKGLEWV |
| ScFv | | SGIVYSGSTYYAASVKGRFTISRDNSRNTLYLQMNSLRPEDTAIYYCS |
| domain | | AHGGESDVWGQGTTVTVSSASGGGGSGGRASGGGGSEIVLTQSPGTLS |
| | | LSPGERATLSCRASQSVGSTNLAWYQQKPGQAPRLLIYDASNRATGIP |
| | | DRFSGGGSGTDFTLTISRLEPEDFAVYYCQQYGSSPPWTFGQGTKVEI |
| | | K |
| 139107- nt | 349 | GAAGTGCAATTGGTGGAGACTGGAGGAGGAGTGGTGCAACCTGGAGGA |
| ScFv | | AGCCTGAGACTGTCATGCGCGGTGTCGGGCTTCGCCCTCTCCAACCAC |
| domain | | GGAATGTCCTGGGTCCGCCGGGCCCCTGGGAAAGGACTTGAATGGGTG |
| | | TCCGGCATCGTGTACTCGGGTTCCACCTACTACGCGGCCTCAGTGAAG |
| | | GGCCGGTTTACTATTAGCCGCGACAACTCCAGAAACACACTGTACCTC |
| | | CAAATGAACTCGCTGCGGCCGGAAGATACCGCTATCTACTACTGCTCC |
| | | GCCCATGGGGGAGAGTCGGACGTCTGGGGACAGGGCACCACTGTCACT |
| | | GTGTCCAGCGCTTCCGGCGGTGGTGGAAGCGGGGGACGGGCCTCAGGA |
| | | GGCGGTGGCAGCGAGATTGTGCTGACCCAGTCCCCCGGGACCCTGAGC |
| | | CTGTCCCCGGGAGAAAGGGCCACCCTCTCCTGTCGGGCATCCCAGTCC |
| | | GTGGGGTCTACTAACCTTGCATGGTACCAGCAGAAGCCCGGCCAGGCC |
| | | CCTCGCCTGCTGATCTACGACGCGTCCAATAGAGCCACCGGCATCCCG |
| | | GATCGCTTCAGCGGAGGCGGATCGGGCACCGACTTCACCCTCACCATT |
| | | TCAAGGCTGGAACCGGAGGACTTCGCCGTGTACTACTGCCAGCAGTAT |
| | | GGTTCGTCCCCACCCTGGACGTTCGGCCAGGGGACTAAGGTCGAGATC |
| | | AAG |
| 139107- aa | 350 | EVQLVETGGGVVQPGGSLRLSCAVSGFALSNHGMSWVRRAPGKGLEWV |
| VH | | SGIVYSGSTYYAASVKGRFTISRDNSRNTLYLQMNSLRPEDTAIYYCS |
| | | AHGGESDVWGQGTTVTVSS |
| 139107- aa | 351 | EIVLTQSPGTLSLSPGERATLSCRASQSVGSTNLAWYQQKPGQAPRLL |
| VL | | IYDASNRATGIPDRFSGGGSGTDFTLTISRLEPEDFAVYYCQQYGSSP |
| | | PWTFGQGTKVEIK |
| 139107- aa | 352 | MALPVTALLLPLALLLHAARPEVQLVETGGGVVQPGGSLRLSCAVSGF |
| Full CAR | | ALSNHGMSWVRRAPGKGLEWVSGIVYSGSTYYAASVKGRFTISRDNSR |
| | | NTLYLQMNSLRPEDTAIYYCSAHGGESDVWGQGTTVTVSSASGGGGSG |
| | | GRASGGGGSEIVLTQSPGTLSLSPGERATLSCRASQSVGSTNLAWYQQ |
| | | KPGQAPRLLIYDASNRATGIPDRFSGGGSGTDFTLTISRLEPEDFAVY |
| | | YCQQYGSSPPWTFGQGTKVEIKTTTPAPRPPTPAPTIASQPLSLRPEA |
| | | CRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAP |
| | | AYKQGQNQLYNELNLGRREEYDVLDKRRGRDPEMGGKPRRKNPQEGLY |
| | | NELQKDKMAEAYSEIGMKGERRRGKGHDGLYQGLSTATKDTYDALHMQ |
| | | ALPPR |
| 139107- nt | 353 | ATGGCCCTCCCTGTCACCGCCCTGCTGCTTCCGCTGGCTCTTCTGCTC |
| Full CAR | | CACGCCGCTCGGCCCGAAGTGCAATTGGTGGAGACTGGAGGAGGAGTG |
| | | GTGCAACCTGGAGGAAGCCTGAGACTGTCATGCGCGGTGTCGGGCTTC |
| | | GCCCTCTCCAACCACGGAATGTCCTGGGTCCGCCGGGCCCCTGGGAAA |
| | | GGACTTGAATGGGTGTCCGGCATCGTGTACTCGGGTTCCACCTACTAC |
| | | GCGGCCTCAGTGAAGGGCCGGTTTACTATTAGCCGCGACAACTCCAGA |
| | | AACACACTGTACCTCCAAATGAACTCGCTGCGGCCGGAAGATACCGCT |
| | | ATCTACTACTGCTCCGCCCATGGGGGAGAGTCGGACGTCTGGGGACAG |
| | | GGCACCACTGTCACTGTGTCCAGCGCTTCCGGCGGTGGTGGAAGCGGG |
| | | GGACGGGCCTCAGGAGGCGGTGGCAGCGAGATTGTGCTGACCCAGTCC |
| | | CCCGGGACCCTGAGCCTGTCCCCGGGAGAAAGGGCCACCCTCTCCTGT |
| | | CGGGCATCCCAGTCCGTGGGGTCTACTAACCTTGCATGGTACCAGCAG |
| | | AAGCCCGGCCAGGCCCCTCGCCTGCTGATCTACGACGCGTCCAATAGA |
| | | GCCACCGGCATCCCGGATCGCTTCAGCGGAGGCGGATCGGGCACCGAC |
| | | TTCACCCTCACCATTTCAAGGCTGGAACCGGAGGACTTCGCCGTGTAC |
| | | TACTGCCAGCAGTATGGTTCGTCCCCACCCTGGACGTTCGGCCAGGGG |
| | | ACTAAGGTCGAGATCAAGACCACTACCCCAGCACCGAGGCCACCCACC |
| | | CCGGCTCCTACCATCGCCTCCCAGCCTCTGTCCCTGCGTCCGGAGGCA |
| | | TGTAGACCCGCAGCTGGTGGGGCCGTGCATACCCGGGGTCTTGACTTC |
| | | GCCTGCGATATCTACATTTGGGCCCCTCTGGCTGGTACTTGCGGGGTC |
| | | CTGCTGCTTTCACTCGTGATCACTCTTTACTGTAAGCGCGGTCGGAAG |
| | | AAGCTGCTGTACATCTTTAAGCAACCCTTCATGAGGCCTGTGCAGACT |
| | | ACTCAAGAGGAGGACGGCTGTTCATGCCGGTTCCCAGAGGAGGAGGAA |
| | | GGCGGCTGCGAACTGCGCGTGAAATTCAGCCGCAGCGCAGATGCTCCA |
| | | GCCTACAAGCAGGGGCAGAACCAGCTCTACAACGAACTCAATCTTGGT |
| | | CGGAGAGAGGAGTACGACGTGCTGGACAAGCGGAGAGGACGGGACCCA |
| | | GAAATGGGCGGGAAGCCGCGCAGAAAGAATCCCCAAGAGGGCCTGTAC |
| | | AACGAGCTCCAAAAGGATAAGATGGCAGAAGCCTATAGCGAGATTGGT |
| | | ATGAAAGGGGAACGCAGAAGAGGCAAAGGCCACGACGGACTGTACCAG |
| | | GGACTCAGCACCGCCACCAAGGACACCTATGACGCTCTTCACATGCAG |
| | | GCCCTGCCGCCTCGG |

139108

| | | |
|---|---|---|
| 139108- aa | 354 | QVQLVESGGGLVKPGGSLRLSCAASGFTFSDYYMSWIRQAPGKGLEWV |
| ScFv | | SYISSSGSTIYYADSVKGRFTISRDNAKNSLYLQMNSLRAEDTAVYYC |
| domain | | ARESGDGMDVWGQGTTVTVSSASGGGGSGGRASGGGGSDIQMTQSPSS |
| | | LSASVGDRVTITCRASQSISSYLNWYQQKPGKAPKLLIYAASSLQSGV |
| | | PSRFSGSGSGTDFTLTISSLQPEDFATYYCQQSYTLAFGQGTKVDIK |
| 139108- nt | 355 | CAAGTGCAACTCGTGGAATCTGGTGGAGGACTCGTGAAACCTGGAGGA |
| ScFv | | TCATTGAGACTGTCATGCGCGGCCTCGGGATTCACGTTCTCCGATTAC |
| domain | | TACATGAGCTGGATTCGCCAGGCTCCGGGGAAGGGACTGGAATGGGTG |
| | | TCCTACATTTCCTCATCCGGCTCCACCATCTACTACGCGGACTCCGTG |
| | | AAGGGGAGATTCACCATTAGCCGCGATAACGCCAAGAACAGCCTGTAC |
| | | CTTCAGATGAACTCCCTGCGGGCTGAAGATACTGCCGTCTACTACTGC |
| | | GCAAGGGAGAGCGGAGATGGGATGGACGTCTGGGGACAGGGTACCACT |
| | | GTGACCGTGTCGTCGGCCTCCGGCGGAGGGGGTTCGGGTGGAAGGGCC |
| | | AGCGGCGGCGGAGGCAGCGACATCCAGATGACCCAGTCCCCCTCATCG |
| | | CTGTCCGCCTCCGTGGGCGACCGCGTCACCATCACATGCCGGGCCTCA |
| | | CAGTCGATCTCCTCCTACCTCAATTGGTATCAGCAGAAGCCCGGAAAG |
| | | GCCCCTAAGCTTCTGATCTACGCAGCGTCCTCCCTGCAATCCGGGGTC |
| | | CCATCTCGGTTCTCCGGCTCGGGCAGCGGTACCGACTTCACTCTGACC |
| | | ATCTCGAGCCTGCAGCCGGAGGACTTCGCCACTTACTACTGTCAGCAA |
| | | AGCTACACCCTCGCGTTTGGCCAGGGCACCAAAGTGGACATCAAG |
| 139108- aa | 356 | QVQLVESGGGLVKPGGSLRLSCAASGFTFSDYYMSWIRQAPGKGLEWV |
| VH | | SYISSSGSTIYYADSVKGRFTISRDNAKNSLYLQMNSLRAEDTAVYYC |
| | | ARESGDGMDVWGQGTTVTVSS |
| 139108- aa | 357 | DIQMTQSPSSLSASVGDRVTITCRASQSISSYLNWYQQKPGKAPKLLI |
| VL | | YAASSLQSGVPSRFSGSGSGTDFTLTISSLQPEDFATYYCQQSYTLAF |
| | | GQGTKVDIK |
| 139108- aa | 358 | MALPVTALLLPLALLLHAARPQVQLVESGGGLVKPGGSLRLSCAASGF |
| Full CAR | | TFSDYYMSWIRQAPGKGLEWVSYISSSGSTIYYADSVKGRFTISRDNA |
| | | KNSLYLQMNSLRAEDTAVYYCARESGDGMDVWGQGTTVTVSSASGGGG |
| | | SGGRASGGGGSDIQMTQSPSSLSASVGDRVTITCRASQSISSYLNWYQ |
| | | QKPGKAPKLLIYAASSLQSGVPSRFSGSGSGTDFTLTISSLQPEDFAT |
| | | YYCQQSYTLAFGQGTKVDIKTTTPAPRPPTPAPTIASQPLSLRPEACR |
| | | PAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQAL |
| | | PPR |
| 139108- nt | 359 | ATGGCCCTCCCTGTCACCGCCCTGCTGCTTCCGCTGGCTCTTCTGCTC |
| Full CAR | | CACGCCGCTCGGCCCCAAGTGCAACTCGTGGAATCTGGTGGAGGACTC |
| | | GTGAAACCTGGAGGATCATTGAGACTGTCATGCGCGGCCTCGGGATTC |
| | | ACGTTCTCCGATTACTACATGAGCTGGATTCGCCAGGCTCCGGGGAAG |
| | | GGACTGGAATGGGTGTCCTACATTTCCTCATCCGGCTCCACCATCTAC |
| | | TACGCGGACTCCGTGAAGGGGAGATTCACCATTAGCCGCGATAACGCC |
| | | AAGAACAGCCTGTACCTTCAGATGAACTCCCTGCGGGCTGAAGATACT |
| | | GCCGTCTACTACTGCGCAAGGGAGAGCGGAGATGGGATGGACGTCTGG |
| | | GGACAGGGTACCACTGTGACCGTGTCGTCGGCCTCCGGCGGAGGGGGT |
| | | TCGGGTGGAAGGGCCAGCGGCGGCGGAGGCAGCGACATCCAGATGACC |
| | | CAGTCCCCCTCATCGCTGTCCGCCTCCGTGGGCGACCGCGTCACCATC |
| | | ACATGCCGGGCCTCACAGTCGATCTCCTCCTACCTCAATTGGTATCAG |
| | | CAGAAGCCCGGAAAGGCCCCTAAGCTTCTGATCTACGCAGCGTCCTCC |
| | | CTGCAATCCGGGGTCCCATCTCGGTTCTCCGGCTCGGGCAGCGGTACC |
| | | GACTTCACTCTGACCATCTCGAGCCTGCAGCCGGAGGACTTCGCCACT |
| | | TACTACTGTCAGCAAAGCTACACCCTCGCGTTTGGCCAGGGCACCAAA |
| | | GTGGACATCAAGACCACTACCCCAGCACCGAGGCCACCCACCCCGGCT |
| | | CCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGC |
| | | GATATCTACATTTGGGCCCCTCTGGCTGGTACTTGCGGGGTCCTGCTG |
| | | CTTTCACTCGTGATCACTCTTTACTGTAAGCGCGGTCGGAAGAAGCTG |
| | | CTGTACATCTTTAAGCAACCCTTCATGAGGCCTGTGCAGACTACTCAA |
| | | GAGGAGGACGGCTGTTCATGCCGGTTCCCAGAGGAGGAGGAAGGCGGC |
| | | TGCGAACTGCGCGTGAAATTCAGCCGCAGCGCAGATGCTCCAGCCTAC |
| | | AAGCAGGGGCAGAACCAGCTCTACAACGAACTCAATCTTGGTCGGAGA |
| | | GAGGAGTACGACGTGCTGGACAAGCGGAGAGGACGGGACCCAGAAATG |
| | | GGCGGGAAGCCGCGCAGAAAGAATCCCCAAGAGGGCCTGTACAACGAG |
| | | CTCCAAAAGGATAAGATGGCAGAAGCCTATAGCGAGATTGGTATGAAA |
| | | GGGGAACGCAGAAGAGGCAAAGGCCACGACGGACTGTACCAGGGACTC |
| | | AGCACCGCCACCAAGGACACCTATGACGCTCTTCACATGCAGGCCCTG |
| | | CCGCCTCGG |

139110

| | | |
|---|---|---|
| 139110- aa | 360 | QVQLVQSGGGLVKPGGSLRLSCAASGFTFSDYYMSWIRQAPGKGLEWV |
| ScFv | | SYISSSGNTIYYADSVKGRFTISRDNAKNSLYLQMNSLRAEDTAVYYC |
| domain | | ARSTMVREDYWGQGTLVTVSSASGGGGSGGRASGGGGSDIVLTQSPLS |
| | | LPVTLGQPASISCKSSESLVHNSGKTYLNWFHQRPGQSPRRLIYEVSN |
| | | RDSGVPDRFTGSGSGTDFTLKISRVEAEDVGVYYCMQGTHWPGTFGQG |
| | | TKLEIK |
| 139110- nt | 361 | CAAGTGCAACTGGTGCAAAGCGGAGGAGGATTGGTCAAACCCGGAGGA |
| ScFv | | AGCCTGAGACTGTCATGCGCGGCCTCTGGATTCACCTTCTCCGATTAC |
| domain | | TACATGTCATGGATCAGACAGGCCCCGGGGAAGGGCCTCGAATGGGTG |
| | | TCCTACATCTCGTCCTCCGGGAACACCATCTACTACGCCGACAGCGTG |
| | | AAGGGCCGCTTTACCATTTCCCGCGACAACGCAAAGAACTCGCTGTAC |
| | | CTTCAGATGAATTCCCTGCGGGCTGAAGATACCGCGGTGTACTATTGC |
| | | GCCCGGTCCACTATGGTCCGGGAGGACTACTGGGGACAGGGCACACTC |
| | | GTGACCGTGTCCAGCGCGAGCGGGGGTGGAGGCAGCGGTGGACGCGCC |
| | | TCCGGCGGCGGCGGTTCAGACATCGTGCTGACTCAGTCGCCCCTGTCG |
| | | CTGCCGGTCACCCTGGGCCAACCGGCCTCAATTAGCTGCAAGTCCTCG |
| | | GAGAGCCTGGTGCACAACTCAGGAAAGACTTACCTGAACTGGTTCCAT |
| | | CAGCGGCCTGGACAGTCCCCACGGAGGCTCATCTATGAAGTGTCCAAC |
| | | AGGGATTCGGGGGTGCCCGACCGCTTCACTGGCTCCGGGTCCGGCACC |
| | | GACTTCACCTTGAAAATCTCCAGAGTGGAAGCCGAGGACGTGGGCGTG |
| | | TACTACTGTATGCAGGGTACCCACTGGCCTGGAACCTTTGGACAAGGA |
| | | ACTAAGCTCGAGATTAAG |
| 139110- aa | 362 | QVQLVQSGGGLVKPGGSLRLSCAASGFTFSDYYMSWIRQAPGKGLEWV |
| VH | | SYISSSGNTIYYADSVKGRFTISRDNAKNSLYLQMNSLRAEDTAVYYC |
| | | ARSTMVREDYWGQGTLVTVSS |
| 139110- aa | 363 | DIVLTQSPLSLPVTLGQPASISCKSSESLVHNSGKTYLNWFHQRPGQS |
| VL | | PRRLIYEVSNRDSGVPDRFTGSGSGTDFTLKISRVEAEDVGVYYCMQG |
| | | THWPGTFGQGTKLEIK |
| 139110- aa | 364 | MALPVTALLLPLALLLHAARPQVQLVQSGGGLVKPGGSLRLSCAASGF |
| Full CAR | | TFSDYYMSWIRQAPGKGLEWVSYISSSGNTIYYADSVKGRFTISRDNA |
| | | KNSLYLQMNSLRAEDTAVYYCARSTMVREDYWGQGTLVTVSSASGGGG |
| | | SGGRASGGGGSDIVLTQSPLSLPVTLGQPASISCKSSESLVHNSGKTY |
| | | LNWFHQRPGQSPRRLIYEVSNRDSGVPDRFTGSGSGTDFTLKISRVEA |
| | | EDVGVYYCMQGTHWPGTFGQGTKLEIKTTTPAPRPPTPAPTIASQPLS |
| | | LRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSR |
| | | SADAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDTYD |
| | | ALHMQALPPR |
| 139110- nt | 365 | ATGGCCCTCCCTGTCACCGCCCTGCTGCTTCCGCTGGCTCTTCTGCTC |
| Full CAR | | CACGCCGCTCGGCCCCAAGTGCAACTGGTGCAAAGCGGAGGAGGATTG |
| | | GTCAAACCCGGAGGAAGCCTGAGACTGTCATGCGCGGCCTCTGGATTC |
| | | ACCTTCTCCGATTACTACATGTCATGGATCAGACAGGCCCCGGGGAAG |
| | | GGCCTCGAATGGGTGTCCTACATCTCGTCCTCCGGGAACACCATCTAC |
| | | TACGCCGACAGCGTGAAGGGCCGCTTTACCATTTCCCGCGACAACGCA |
| | | AAGAACTCGCTGTACCTTCAGATGAATTCCCTGCGGGCTGAAGATACC |
| | | GCGGTGTACTATTGCGCCCGGTCCACTATGGTCCGGGAGGACTACTGG |
| | | GGACAGGGCACACTCGTGACCGTGTCCAGCGCGAGCGGGGGTGGAGGC |
| | | AGCGGTGGACGCGCCTCCGGCGGCGGCGGTTCAGACATCGTGCTGACT |
| | | CAGTCGCCCCTGTCGCTGCCGGTCACCCTGGGCCAACCGGCCTCAATT |
| | | AGCTGCAAGTCCTCGGAGAGCCTGGTGCACAACTCAGGAAAGACTTAC |
| | | CTGAACTGGTTCCATCAGCGGCCTGGACAGTCCCCACGGAGGCTCATC |
| | | TATGAAGTGTCCAACAGGGATTCGGGGGTGCCCGACCGCTTCACTGGC |
| | | TCCGGGTCCGGCACCGACTTCACCTTGAAAATCTCCAGAGTGGAAGCC |
| | | GAGGACGTGGGCGTGTACTACTGTATGCAGGGTACCCACTGGCCTGGA |
| | | ACCTTTGGACAAGGAACTAAGCTCGAGATTAAGACCACTACCCCAGCA |
| | | CCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACC |
| | | CGGGGTCTTGACTTCGCCTGCGATATCTACATTTGGGCCCCTCTGGCT |
| | | GGTACTTGCGGGGTCCTGCTGCTTTCACTCGTGATCACTCTTTACTGT |
| | | AAGCGCGGTCGGAAGAAGCTGCTGTACATCTTTAAGCAACCCTTCATG |
| | | AGGCCTGTGCAGACTACTCAAGAGGAGGACGGCTGTTCATGCCGGTTC |
| | | CCAGAGGAGGAGGAAGGCGGCTGCGAACTGCGCGTGAAATTCAGCCGC |
| | | AGCGCAGATGCTCCAGCCTACAAGCAGGGGCAGAACCAGCTCTACAAC |
| | | GAACTCAATCTTGGTCGGAGAGAGGAGTACGACGTGCTGGACAAGCGG |
| | | AGAGGACGGGACCCAGAAATGGGCGGGAAGCCGCGCAGAAAGAATCCC |
| | | CAAGAGGGCCTGTACAACGAGCTCCAAAAGGATAAGATGGCAGAAGCC |
| | | TATAGCGAGATTGGTATGAAAGGGGAACGCAGAAGAGGCAAAGGCCAC |
| | | GACGGACTGTACCAGGGACTCAGCACCGCCACCAAGGACACCTATGAC |
| | | GCTCTTCACATGCAGGCCCTGCCGCCTCGG |

139112

| | | |
|---|---|---|
| 139112- aa | 366 | QVQLVESGGGLVQPGGSLRLSCAVSGFALSNHGMSWVRRAPGKGLEWV |
| ScFv | | SGIVYSGSTYYAASVKGRFTISRDNSRNTLYLQMNSLRPEDTAIYYCS |
| domain | | AHGGESDVWGQGTTVTVSSASGGGGSGGRASGGGGSDIRLTQSPSPLS |
| | | ASVGDRVTITCQASEDINKFLNWYHQTPGKAPKLLIYDASTLQTGVPS |
| | | RFSGSGSGTDFTLTINSLQPEDIGTYYCQQYESLPLTFGGGTKVEIK |
| 139112- nt | 367 | CAAGTGCAACTCGTGGAATCTGGTGGAGGACTCGTGCAACCCGGTGGA |
| ScFv | | AGCCTTAGGCTGTCGTGCGCCGTCAGCGGGTTTGCTCTGAGCAACCAT |
| domain | | GGAATGTCCTGGGTCCGCCGGGCACCGGGAAAAGGGCTGGAATGGGTG |
| | | TCCGGCATCGTGTACAGCGGGTCAACCTATTACGCCGCGTCCGTGAAG |
| | | GGCAGATTCACTATCTCAAGAGACAACAGCCGGAACACCCTGTACTTG |
| | | CAAATGAATTCCCTGCGCCCCGAGGACACCGCCATCTACTACTGCTCC |
| | | GCCCACGGAGGAGAGTCGGACGTGTGGGGCCAGGGAACGACTGTGACT |
| | | GTGTCCAGCGCATCAGGAGGGGGTGGTTCGGGCGGCCGGGCCTCGGGG |
| | | GGAGGAGGTTCCGACATTCGGCTGACCCAGTCCCCGTCCCCACTGTCG |
| | | GCCTCCGTCGGCGACCGCGTGACCATCACTTGTCAGGCGTCCGAGGAC |
| | | ATTAACAAGTTCCTGAACTGGTACCACCAGACCCCTGGAAAGGCCCCC |
| | | AAGCTGCTGATCTACGATGCCTCGACCCTTCAAACTGGAGTGCCTAGC |
| | | CGGTTCTCCGGGTCCGGCTCCGGCACTGATTTCACTCTGACCATCAAC |
| | | TCATTGCAGCCGGAAGATATCGGGACCTACTATTGCCAGCAGTACGAA |
| | | TCCCTCCCGCTCACATTCGGCGGGGGAACCAAGGTCGAGATTAAG |
| 139112- aa | 368 | QVQLVESGGGLVQPGGSLRLSCAVSGFALSNHGMSWVRRAPGKGLEWV |
| VH | | SGIVYSGSTYYAASVKGRFTISRDNSRNTLYLQMNSLRPEDTAIYYCS |
| | | AHGGESDVWGQGTTVTVSS |
| 139112- aa | 369 | DIRLTQSPSPLSASVGDRVTITCQASEDINKFLNWYHQTPGKAPKLLI |
| VL | | YDASTLQTGVPSRFSGSGSGTDFTLTINSLQPEDIGTYYCQQYESLPL |
| | | TFGGGTKVEIK |
| 139112- aa | 370 | MALPVTALLLPLALLLHAARPQVQLVESGGGLVQPGGSLRLSCAVSGF |
| Full CAR | | ALSNHGMSWVRRAPGKGLEWVSGIVYSGSTYYAASVKGRFTISRDNSR |
| | | NTLYLQMNSLRPEDTAIYYCSAHGGESDVWGQGTTVTVSSASGGGGSG |
| | | GRASGGGGSDIRLTQSPSPLSASVGDRVTITCQASEDINKFLNWYHQT |
| | | PGKAPKLLIYDASTLQTGVPSRFSGSGSGTDFTLTINSLQPEDIGTYY |
| | | CQQYESLPLTFGGGTKVEIKTTTPAPRPPTPAPTIASQPLSLRPEACR |
| | | PAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQAL |
| | | PPR |
| 139112- nt | 371 | ATGGCCCTCCCTGTCACCGCCCTGCTGCTTCCGCTGGCTCTTCTGCTC |
| Full CAR | | CACGCCGCTCGGCCCCAAGTGCAACTCGTGGAATCTGGTGGAGGACTC |
| | | GTGCAACCCGGTGGAAGCCTTAGGCTGTCGTGCGCCGTCAGCGGGTTT |
| | | GCTCTGAGCAACCATGGAATGTCCTGGGTCCGCCGGGCACCGGGAAAA |
| | | GGGCTGGAATGGGTGTCCGGCATCGTGTACAGCGGGTCAACCTATTAC |
| | | GCCGCGTCCGTGAAGGGCAGATTCACTATCTCAAGAGACAACAGCCGG |
| | | AACACCCTGTACTTGCAAATGAATTCCCTGCGCCCCGAGGACACCGCC |
| | | ATCTACTACTGCTCCGCCCACGGAGGAGAGTCGGACGTGTGGGGCCAG |
| | | GGAACGACTGTGACTGTGTCCAGCGCATCAGGAGGGGGTGGTTCGGGC |
| | | GGCCGGGCCTCGGGGGGAGGAGGTTCCGACATTCGGCTGACCCAGTCC |
| | | CCGTCCCCACTGTCGGCCTCCGTCGGCGACCGCGTGACCATCACTTGT |
| | | CAGGCGTCCGAGGACATTAACAAGTTCCTGAACTGGTACCACCAGACC |
| | | CCTGGAAAGGCCCCCAAGCTGCTGATCTACGATGCCTCGACCCTTCAA |
| | | ACTGGAGTGCCTAGCCGGTTCTCCGGGTCCGGCTCCGGCACTGATTTC |
| | | ACTCTGACCATCAACTCATTGCAGCCGGAAGATATCGGGACCTACTAT |
| | | TGCCAGCAGTACGAATCCCTCCCGCTCACATTCGGCGGGGGAACCAAG |
| | | GTCGAGATTAAGACCACTACCCCAGCACCGAGGCCACCCACCCCGGCT |
| | | CCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGC |
| | | GATATCTACATTTGGGCCCCTCTGGCTGGTACTTGCGGGGTCCTGCTG |
| | | CTTTCACTCGTGATCACTCTTTACTGTAAGCGCGGTCGGAAGAAGCTG |
| | | CTGTACATCTTTAAGCAACCCTTCATGAGGCCTGTGCAGACTACTCAA |
| | | GAGGAGGACGGCTGTTCATGCCGGTTCCCAGAGGAGGAGGAAGGCGGC |
| | | TGCGAACTGCGCGTGAAATTCAGCCGCAGCGCAGATGCTCCAGCCTAC |
| | | AAGCAGGGGCAGAACCAGCTCTACAACGAACTCAATCTTGGTCGGAGA |
| | | GAGGAGTACGACGTGCTGGACAAGCGGAGAGGACGGGACCCAGAAATG |
| | | GGCGGGAAGCCGCGCAGAAAGAATCCCCAAGAGGGCCTGTACAACGAG |
| | | CTCCAAAAGGATAAGATGGCAGAAGCCTATAGCGAGATTGGTATGAAA |
| | | GGGGAACGCAGAAGAGGCAAAGGCCACGACGGACTGTACCAGGGACTC |
| | | AGCACCGCCACCAAGGACACCTATGACGCTCTTCACATGCAGGCCCTG |
| | | CCGCCTCGG |

139113

| | | |
|---|---|---|
| 139113- aa | 372 | EVQLVETGGGLVQPGGSLRLSCAVSGFALSNHGMSWVRRAPGKGLEWV |
| ScFv | | SGIVYSGSTYYAASVKGRFTISRDNSRNTLYLQMNSLRPEDTAIYYCS |
| domain | | AHGGESDVWGQGTTVTVSSASGGGGSGGRASGGGGSETTLTQSPATLS |
| | | VSPGERATLSCRASQSVGSNLAWYQQKPGQGPRLLIYGASTRATGIPA |
| | | RFSGSGSGTEFTLTISSLQPEDFAVYYCQQYNDWLPVTFGQGTKVEIK |
| 139113- nt | 373 | GAAGTGCAATTGGTGGAAACTGGAGGAGGACTTGTGCAACCTGGAGGA |
| ScFv | | TCATTGCGGCTCTCATGCGCTGTCTCCGGCTTCGCCCTGTCAAATCAC |
| domain | | GGGATGTCGTGGGTCAGACGGGCCCCGGGAAAGGGTCTGGAATGGGTG |
| | | TCGGGGATTGTGTACAGCGGCTCCACCTACTACGCCGCTTCGGTCAAG |
| | | GGCCGCTTCACTATTTCACGGGACAACAGCCGCAACACCCTCTATCTG |
| | | CAAATGAACTCTCTCCGCCCGGAGGATACCGCCATCTACTACTGCTCC |
| | | GCACACGGCGGCGAATCCGACGTGTGGGGACAGGGAACCACTGTCACC |
| | | GTGTCGTCCGCATCCGGTGGCGGAGGATCGGGTGGCCGGGCCTCCGGG |
| | | GGCGGCGGCAGCGAGACTACCCTGACCCAGTCCCCTGCCACTCTGTCC |
| | | GTGAGCCCGGGAGAGAGAGCCACCCTTAGCTGCCGGGCCAGCCAGAGC |
| | | GTGGGCTCCAACCTGGCCTGGTACCAGCAGAAGCCAGGACAGGGTCCC |
| | | AGGCTGCTGATCTACGGAGCCTCCACTCGCGCGACCGGCATCCCCGCG |
| | | AGGTTCTCCGGGTCGGGTTCCGGGACCGAGTTCACCCTGACCATCTCC |
| | | TCCCTCCAACCGGAGGACTTCGCGGTGTACTACTGTCAGCAGTACAAC |
| | | GATTGGCTGCCCGTGACATTTGGACAGGGGACGAAGGTGGAAATCAAA |
| 139113- aa | 374 | EVQLVETGGGLVQPGGSLRLSCAVSGFALSNHGMSWVRRAPGKGLEWV |
| VH | | SGIVYSGSTYYAASVKGRFTISRDNSRNTLYLQMNSLRPEDTAIYYCS |
| | | AHGGESDVWGQGTTVTVSS |
| 139113- aa | 375 | ETTLTQSPATLSVSPGERATLSCRASQSVGSNLAWYQQKPGQGPRLLI |
| VL | | YGASTRATGIPARFSGSGSGTEFTLTISSLQPEDFAVYYCQQYNDWLP |
| | | VTFGQGTKVEIK |
| 139113- aa | 376 | MALPVTALLLPLALLLHAARPEVQLVETGGGLVQPGGSLRLSCAVSGF |
| Full CAR | | ALSNHGMSWVRRAPGKGLEWVSGIVYSGSTYYAASVKGRFTISRDNSR |
| | | NTLYLQMNSLRPEDTAIYYCSAHGGESDVWGQGTTVTVSSASGGGGSG |
| | | GRASGGGGSETTLTQSPATLSVSPGERATLSCRASQSVGSNLAWYQQK |
| | | PGQGPRLLIYGASTRATGIPARFSGSGSGTEFTLTISSLQPEDFAVYY |
| | | CQQYNDWLPVTFGQGTKVEIKTTTPAPRPPTPAPTIASQPLSLRPEAC |
| | | RPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPA |
| | | YKQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQA |
| | | LPPR |
| 139113- nt | 377 | ATGGCCCTCCCTGTCACCGCCCTGCTGCTTCCGCTGGCTCTTCTGCTC |
| Full CAR | | CACGCCGCTCGGCCCGAAGTGCAATTGGTGGAAACTGGAGGAGGACTT |
| | | GTGCAACCTGGAGGATCATTGCGGCTCTCATGCGCTGTCTCCGGCTTC |
| | | GCCCTGTCAAATCACGGGATGTCGTGGGTCAGACGGGCCCCGGGAAAG |
| | | GGTCTGGAATGGGTGTCGGGGATTGTGTACAGCGGCTCCACCTACTAC |
| | | GCCGCTTCGGTCAAGGGCCGCTTCACTATTTCACGGGACAACAGCCGC |
| | | AACACCCTCTATCTGCAAATGAACTCTCTCCGCCCGGAGGATACCGCC |
| | | ATCTACTACTGCTCCGCACACGGCGGCGAATCCGACGTGTGGGGACAG |
| | | GGAACCACTGTCACCGTGTCGTCCGCATCCGGTGGCGGAGGATCGGGT |
| | | GGCCGGGCCTCCGGGGGCGGCGGCAGCGAGACTACCCTGACCCAGTCC |
| | | CCTGCCACTCTGTCCGTGAGCCCGGGAGAGAGAGCCACCCTTAGCTGC |
| | | CGGGCCAGCCAGAGCGTGGGCTCCAACCTGGCCTGGTACCAGCAGAAG |
| | | CCAGGACAGGGTCCCAGGCTGCTGATCTACGGAGCCTCCACTCGCGCG |
| | | ACCGGCATCCCCGCGAGGTTCTCCGGGTCGGGTTCCGGGACCGAGTTC |
| | | ACCCTGACCATCTCCTCCCTCCAACCGGAGGACTTCGCGGTGTACTAC |
| | | TGTCAGCAGTACAACGATTGGCTGCCCGTGACATTTGGACAGGGGACG |
| | | AAGGTGGAAATCAAAACCACTACCCCAGCACCGAGGCCACCCACCCCG |
| | | GCTCCTACCATCGCCTCCCAGCCTCTGTCCCTGCGTCCGGAGGCATGT |
| | | AGACCCGCAGCTGGTGGGGCCGTGCATACCCGGGGTCTTGACTTCGCC |
| | | TGCGATATCTACATTTGGGCCCCTCTGGCTGGTACTTGCGGGGTCCTG |
| | | CTGCTTTCACTCGTGATCACTCTTTACTGTAAGCGCGGTCGGAAGAAG |
| | | CTGCTGTACATCTTTAAGCAACCCTTCATGAGGCCTGTGCAGACTACT |
| | | CAAGAGGAGGACGGCTGTTCATGCCGGTTCCCAGAGGAGGAGGAAGGC |
| | | GGCTGCGAACTGCGCGTGAAATTCAGCCGCAGCGCAGATGCTCCAGCC |
| | | TACAAGCAGGGGCAGAACCAGCTCTACAACGAACTCAATCTTGGTCGG |
| | | AGAGAGGAGTACGACGTGCTGGACAAGCGGAGAGGACGGGACCCAGAA |
| | | ATGGGCGGGAAGCCGCGCAGAAAGAATCCCCAAGAGGGCCTGTACAAC |
| | | GAGCTCCAAAAGGATAAGATGGCAGAAGCCTATAGCGAGATTGGTATG |
| | | AAAGGGGAACGCAGAAGAGGCAAAGGCCACGACGGACTGTACCAGGGA |
| | | CTCAGCACCGCCACCAAGGACACCTATGACGCTCTTCACATGCAGGCC |
| | | CTGCCGCCTCGG |

139114

| | | |
|---|---|---|
| 139114- aa | 378 | EVQLVESGGGLVQPGGSLRLSCAVSGFALSNHGMSWVRRAPGKGLEWV |
| ScFv | | SGIVYSGSTYYAASVKGRFTISRDNSRNTLYLQMNSLRPEDTAIYYCS |
| domain | | AHGGESDVWGQGTTVTVSSASGGGGSGGRASGGGGSEIVLTQSPGTLS |
| | | LSPGERATLSCRASQSIGSSSLAWYQQKPGQAPRLLMYGASSRASGIP |
| | | DRFSGSGSGTDFTLTISRLEPEDFAVYYCQQYAGSPPFTFGQGTKVEI |
| | | K |
| 139114- nt | 379 | GAAGTGCAATTGGTGGAATCTGGTGGAGGACTTGTGCAACCTGGAGGA |
| ScFv | | TCACTGAGACTGTCATGCGCGGTGTCCGGTTTTGCCCTGAGCAATCAT |
| domain | | GGGATGTCGTGGGTCCGGCGCGCCCCCGGAAAGGGTCTGGAATGGGTG |
| | | TCGGGTATCGTCTACTCCGGGAGCACTTACTACGCCGCGAGCGTGAAG |
| | | GGCCGCTTCACCATTTCCCGCGATAACTCCCGCAACACCCTGTACTTG |
| | | CAAATGAACTCGCTCCGGCCTGAGGACACTGCCATCTACTACTGCTCC |
| | | GCACACGGAGGAGAATCCGACGTGTGGGGCCAGGGAACTACCGTGACC |
| | | GTCAGCAGCGCCTCCGGCGGCGGGGGCTCAGGCGGACGGGCTAGCGGC |
| | | GGCGGTGGCTCCGAGATCGTGCTGACCCAGTCGCCTGGCACTCTCTCG |
| | | CTGAGCCCCGGGGAAAGGGCAACCCTGTCCTGTCGGGCCAGCCAGTCC |
| | | ATTGGATCATCCTCCCTCGCCTGGTATCAGCAGAAACCGGGACAGGCT |
| | | CCGCGGCTGCTTATGTATGGGGCCAGCTCAAGAGCCTCCGGCATTCCC |
| | | GACCGGTTCTCCGGGTCCGGTTCCGGCACCGATTTCACCCTGACTATC |
| | | TCGAGGCTGGAGCCAGAGGACTTCGCCGTGTACTACTGCCAGCAGTAC |
| | | GCGGGGTCCCCGCCGTTCACGTTCGGACAGGGAACCAAGGTCGAGATC |
| | | AAG |
| 139114- aa | 380 | EVQLVESGGGLVQPGGSLRLSCAVSGFALSNHGMSWVRRAPGKGLEWV |
| VH | | SGIVYSGSTYYAASVKGRFTISRDNSRNTLYLQMNSLRPEDTAIYYCS |
| | | AHGGESDVWGQGTTVTVSS |
| 139114- aa | 381 | EIVLTQSPGTLSLSPGERATLSCRASQSIGSSSLAWYQQKPGQAPRLL |
| VL | | MYGASSRASGIPDRFSGSGSGTDFTLTISRLEPEDFAVYYCQQYAGSP |
| | | PFTFGQGTKVEIK |
| 139114- aa | 382 | MALPVTALLLPLALLLHAARPEVQLVESGGGLVQPGGSLRLSCAVSGF |
| Full CAR | | ALSNHGMSWVRRAPGKGLEWVSGIVYSGSTYYAASVKGRFTISRDNSR |
| | | NTLYLQMNSLRPEDTAIYYCSAHGGESDVWGQGTTVTVSSASGGGGSG |
| | | GRASGGGGSEIVLTQSPGTLSLSPGERATLSCRASQSIGSSSLAWYQQ |
| | | KPGQAPRLLMYGASSRASGIPDRFSGSGSGTDFTLTISRLEPEDFAVY |
| | | YCQQYAGSPPFTFGQGTKVEIKTTTPAPRPPTPAPTIASQPLSLRPEA |
| | | CRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAP |
| | | AYKQGQNQLYNELNLGRREEYDVLDKRRGRDPEMGGKPRRKNPQEGLY |
| | | NELQKDKMAEAYSEIGMKGERRRGKGHDGLYQGLSTATKDTYDALHMQ |
| | | ALPPR |
| 139114- nt | 383 | ATGGCCCTCCCTGTCACCGCCCTGCTGCTTCCGCTGGCTCTTCTGCTC |
| Full CAR | | CACGCCGCTCGGCCCGAAGTGCAATTGGTGGAATCTGGTGGAGGACTT |
| | | GTGCAACCTGGAGGATCACTGAGACTGTCATGCGCGGTGTCCGGTTTT |
| | | GCCCTGAGCAATCATGGGATGTCGTGGGTCCGGCGCGCCCCCGGAAAG |
| | | GGTCTGGAATGGGTGTCGGGTATCGTCTACTCCGGGAGCACTTACTAC |
| | | GCCGCGAGCGTGAAGGGCCGCTTCACCATTTCCCGCGATAACTCCCGC |
| | | AACACCCTGTACTTGCAAATGAACTCGCTCCGGCCTGAGGACACTGCC |
| | | ATCTACTACTGCTCCGCACACGGAGGAGAATCCGACGTGTGGGGCCAG |
| | | GGAACTACCGTGACCGTCAGCAGCGCCTCCGGCGGCGGGGGCTCAGGC |
| | | GGACGGGCTAGCGGCGGCGGTGGCTCCGAGATCGTGCTGACCCAGTCG |
| | | CCTGGCACTCTCTCGCTGAGCCCCGGGGAAAGGGCAACCCTGTCCTGT |
| | | CGGGCCAGCCAGTCCATTGGATCATCCTCCCTCGCCTGGTATCAGCAG |
| | | AAACCGGGACAGGCTCCGCGGCTGCTTATGTATGGGGCCAGCTCAAGA |
| | | GCCTCCGGCATTCCCGACCGGTTCTCCGGGTCCGGTTCCGGCACCGAT |
| | | TTCACCCTGACTATCTCGAGGCTGGAGCCAGAGGACTTCGCCGTGTAC |
| | | TACTGCCAGCAGTACGCGGGGTCCCCGCCGTTCACGTTCGGACAGGGA |
| | | ACCAAGGTCGAGATCAAGACCACTACCCCAGCACCGAGGCCACCCACC |
| | | CCGGCTCCTACCATCGCCTCCCAGCCTCTGTCCCTGCGTCCGGAGGCA |
| | | TGTAGACCCGCAGCTGGTGGGGCCGTGCATACCCGGGGTCTTGACTTC |
| | | GCCTGCGATATCTACATTTGGGCCCCTCTGGCTGGTACTTGCGGGGTC |
| | | CTGCTGCTTTCACTCGTGATCACTCTTTACTGTAAGCGCGGTCGGAAG |
| | | AAGCTGCTGTACATCTTTAAGCAACCCTTCATGAGGCCTGTGCAGACT |
| | | ACTCAAGAGGAGGACGGCTGTTCATGCCGGTTCCCAGAGGAGGAGGAA |
| | | GGCGGCTGCGAACTGCGCGTGAAATTCAGCCGCAGCGCAGATGCTCCA |
| | | GCCTACAAGCAGGGGCAGAACCAGCTCTACAACGAACTCAATCTTGGT |
| | | CGGAGAGAGGAGTACGACGTGCTGGACAAGCGGAGAGGACGGGACCCA |
| | | GAAATGGGCGGGAAGCCGCGCAGAAAGAATCCCCAAGAGGGCCTGTAC |
| | | AACGAGCTCCAAAAGGATAAGATGGCAGAAGCCTATAGCGAGATTGGT |
| | | ATGAAAGGGGAACGCAGAAGAGGCAAAGGCCACGACGGACTGTACCAG |
| | | GGACTCAGCACCGCCACCAAGGACACCTATGACGCTCTTCACATGCAG |
| | | GCCCTGCCGCCTCGG |

149362

| | | |
|---|---|---|
| 149362-aa | 384 | QVQLQESGPGLVKPSETLSLTCTVSGGSISSSYYYWGWIRQPPGKGLE |
| ScFv | | WIGSIYYSGSAYYNPSLKSRVTISVDTSKNQFSLRLSSVTAADTAVYY |
| domain | | CARHWQEWPDAFDIWGQGTMVTVSSGGGGSGGGGSGGGGSETTLTQSP |
| | | AFMSATPGDKVIISCKASQDIDDAMNWYQQKPGEAPLFIIQSATSPVP |
| | | GIPPRFSGSGFGTDFSLTINNIESEDAAYYFCLQHDNFPLTFGQGTKL |
| | | EIK |
| 149362-nt | 385 | CAAGTGCAGCTTCAGGAAAGCGGACCGGGCCTGGTCAAGCCATCCGAA |
| ScFv | | ACTCTCTCCCTGACTTGCACTGTGTCTGGCGGTTCCATCTCATCGTCG |
| domain | | TACTACTACTGGGGCTGGATTAGGCAGCCGCCCGGAAAGGGACTGGAG |
| | | TGGATCGGAAGCATCTACTATTCCGGCTCGGCGTACTACAACCCTAGC |
| | | CTCAAGTCGAGAGTGACCATCTCCGTGGATACCTCCAAGAACCAGTTT |
| | | TCCCTGCGCCTGAGCTCCGTGACCGCCGCTGACACCGCCGTGTACTAC |
| | | TGTGCTCGGCATTGGCAGGAATGGCCCGATGCCTTCGACATTTGGGGC |
| | | CAGGGCACTATGGTCACTGTGTCATCCGGGGGTGGAGGCAGCGGGGGA |
| | | GGAGGGTCCGGGGGGGGAGGTTCAGAGACAACCTTGACCCAGTCACCC |
| | | GCATTCATGTCCGCCACTCCGGGAGACAAGGTCATCATCTCGTGCAAA |
| | | GCGTCCCAGGATATCGACGATGCCATGAATTGGTACCAGCAGAAGCCT |
| | | GGCGAAGCGCCGCTGTTCATTATCCAATCCGCAACCTCGCCCGTGCCT |
| | | GGAATCCCACCGCGGTTCAGCGGCAGCGGTTTCGGAACCGACTTTTCC |
| | | CTGACCATTAACAACATTGAGTCCGAGGACGCCGCCTACTACTTCTGC |
| | | CTGCAACACGACAACTTCCCTCTCACGTTCGGCCAGGGAACCAAGCTG |
| | | GAAATCAAG |
| 149362-aa | 386 | QVQLQESGPGLVKPSETLSLTCTVSGGSISSSYYYWGWIRQPPGKGLE |
| VH | | WIGSIYYSGSAYYNPSLKSRVTISVDTSKNQFSLRLSSVTAADTAVYY |
| | | CARHWQEWPDAFDIWGQGTMVTVSS |
| 149362-aa | 387 | ETTLTQSPAFMSATPGDKVIISCKASQDIDDAMNWYQQKPGEAPLFII |
| VL | | QSATSPVPGIPPRFSGSGFGTDFSLTINNIESEDAAYYFCLQHDNFPL |
| | | TFGQGTKLEIK |
| 149362-aa | 388 | MALPVTALLLPLALLLHAARPQVQLQESGPGLVKPSETLSLTCTVSGG |
| Full CAR | | SISSSYYYWGWIRQPPGKGLEWIGSIYYSGSAYYNPSLKSRVTISVDT |
| | | SKNQFSLRLSSVTAADTAVYYCARHWQEWPDAFDIWGQGTMVTVSSGG |
| | | GGSGGGGSGGGGSETTLTQSPAFMSATPGDKVIISCKASQDIDDAMNW |
| | | YQQKPGEAPLFIIQSATSPVPGIPPRFSGSGFGTDFSLTINNIESEDA |
| | | AYYFCLQHDNFPLTFGQGTKLEIKTTTPAPRPPTPAPTIASQPLSLRP |
| | | EACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSAD |
| | | APAYKQGQNQLYNELNLGRREEYDVLDKRRGRDPEMGGKPRRKNPQEG |
| | | LYNELQKDKMAEAYSEIGMKGERRRGKGHDGLYQGLSTATKDTYDALH |
| | | MQALPPR |
| 149362-nt | 389 | ATGGCCCTCCCTGTCACCGCCCTGCTGCTTCCGCTGGCTCTTCTGCTC |
| Full CAR | | CACGCCGCTCGGCCCCAAGTGCAGCTTCAGGAAAGCGGACCGGGCCTG |
| | | GTCAAGCCATCCGAAACTCTCTCCCTGACTTGCACTGTGTCTGGCGGT |
| | | TCCATCTCATCGTCGTACTACTACTGGGGCTGGATTAGGCAGCCGCCC |
| | | GGAAAGGGACTGGAGTGGATCGGAAGCATCTACTATTCCGGCTCGGCG |
| | | TACTACAACCCTAGCCTCAAGTCGAGAGTGACCATCTCCGTGGATACC |
| | | TCCAAGAACCAGTTTTCCCTGCGCCTGAGCTCCGTGACCGCCGCTGAC |
| | | ACCGCCGTGTACTACTGTGCTCGGCATTGGCAGGAATGGCCCGATGCC |
| | | TTCGACATTTGGGGCCAGGGCACTATGGTCACTGTGTCATCCGGGGGT |
| | | GGAGGCAGCGGGGGAGGAGGGTCCGGGGGGGGAGGTTCAGAGACAACC |
| | | TTGACCCAGTCACCCGCATTCATGTCCGCCACTCCGGGAGACAAGGTC |
| | | ATCATCTCGTGCAAAGCGTCCCAGGATATCGACGATGCCATGAATTGG |
| | | TACCAGCAGAAGCCTGGCGAAGCGCCGCTGTTCATTATCCAATCCGCA |
| | | ACCTCGCCCGTGCCTGGAATCCCACCGCGGTTCAGCGGCAGCGGTTTC |
| | | GGAACCGACTTTTCCCTGACCATTAACAACATTGAGTCCGAGGACGCC |
| | | GCCTACTACTTCTGCCTGCAACACGACAACTTCCCTCTCACGTTCGGC |
| | | CAGGGAACCAAGCTGGAAATCAAGACCACTACCCCAGCACCGAGGCCA |
| | | CCCACCCCGGCTCCTACCATCGCCTCCCAGCCTCTGTCCCTGCGTCCG |
| | | GAGGCATGTAGACCCGCAGCTGGTGGGGCCGTGCATACCCGGGGTCTT |
| | | GACTTCGCCTGCGATATCTACATTTGGGCCCCTCTGGCTGGTACTTGC |
| | | GGGGTCCTGCTGCTTTCACTCGTGATCACTCTTTACTGTAAGCGCGGT |
| | | CGGAAGAAGCTGCTGTACATCTTTAAGCAACCCTTCATGAGGCCTGIG |
| | | CAGACTACTCAAGAGGAGGACGGCTGTTCATGCCGGTTCCCAGAGGAG |
| | | GAGGAAGGCGGCTGCGAACTGCGCGTGAAATTCAGCCGCAGCGCAGAT |
| | | GCTCCAGCCTACAAGCAGGGGCAGAACCAGCTCTACAACGAACTCAAT |
| | | CTTGGTCGGAGAGAGGAGTACGACGTGCTGGACAAGCGGAGAGGACGG |
| | | GACCCAGAAATGGGCGGGAAGCCGCGCAGAAAGAATCCCCAAGAGGGC |
| | | CTGTACAACGAGCTCCAAAAGGATAAGATGGCAGAAGCCTATAGCGAG |
| | | ATTGGTATGAAAGGGGAACGCAGAAGAGGCAAAGGCCACGACGGACTG |
| | | TACCAGGGACTCAGCACCGCCACCAAGGACACCTATGACGCTCTTCAC |
| | | ATGCAGGCCCTGCCGCCTCGG |

149363

| | | |
|---|---|---|
| 149363-aa | 390 | VNLRESGPALVKPTQTLTLTCTFSGFSLRTSGMCVSWIRQPPGKALEW |
| ScFv | | LARIDWDEDKFYSTSLKTRLTISKDTSDNQVVLRMTNMDPADTATYYC |
| domain | | ARSGAGGTSATAFDIWGPGTMVTVSSGGGGSGGGGSGGGGSDIQMTQS |
| | | PSSLSASVGDRVTITCRASQDIYNNLAWFQLKPGSAPRSLMYAANKSQ |
| | | SGVPSRFSGSASGTDFTLTISSLQPEDFATYYCQHYYRFPYSFGQGTK |
| | | LEIK |
| 149363-nt | 391 | CAAGTCAATCTGCGCGAATCCGGCCCCGCCTTGGTCAAGCCTACCCAG |
| ScFv | | ACCCTCACTCTGACCTGTACTTTCTCCGGCTTCTCCCTGCGGACTTCC |
| domain | | GGGATGTGCGTGTCCTGGATCAGACAGCCTCCGGGAAAGGCCCTGGAG |
| | | TGGCTCGCTCGCATTGACTGGGATGAGGACAAGTTCTACTCCACCTCA |
| | | CTCAAGACCAGGCTGACCATCAGCAAAGATACCTCTGACAACCAAGTG |
| | | GTGCTCCGCATGACCAACATGGACCCAGCCGACACTGCCACTTACTAC |
| | | TGCGCGAGGAGCGGAGCGGGCGGAACCTCCGCCACCGCCTTCGATATT |
| | | TGGGGCCCGGGTACCATGGTCACCGTGTCAAGCGGAGGAGGGGGGTCC |
| | | GGGGGCGGCGGTTCCGGGGGAGGCGGATCGGACATTCAGATGACTCAG |
| | | TCACCATCGTCCCTGAGCGCTAGCGTGGGCGACAGAGTGACAATCACT |
| | | TGCCGGGCATCCCAGGACATCTATAACAACCTTGCGTGGTTCCAGCTG |
| | | AAGCCTGGTTCCGCACCGCGGTCACTTATGTACGCCGCCAACAAGAGC |
| | | CAGTCGGGAGTGCCGTCCCGGTTTTCCGGTTCGGCCTCGGGAACTGAC |
| | | TTCACCCTGACGATCTCCAGCCTGCAACCCGAGGATTTCGCCACCTAC |
| | | TACTGCCAGCACTACTACCGCTTTCCCTACTCGTTCGGACAGGGAACC |
| | | AAGCTGGAAATCAAG |
| 149363-aa | 392 | QVNLRESGPALVKPTQTLTLTCTFSGFSLRTSGMCVSWIRQPPGKALE |
| VH | | WLARIDWDEDKFYSTSLKTRLTISKDTSDNQVVLRMTNMDPADTATYY |
| | | CARSGAGGTSATAFDIWGPGTMVTVSS |
| 149363-aa | 393 | DIQMTQSPSSLSASVGDRVTITCRASQDIYNNLAWFQLKPGSAPRSLM |
| VL | | YAANKSQSGVPSRFSGSASGTDFTLTISSLQPEDFATYYCQHYYRFPY |
| | | SFGQGTKLEIK |
| 149363-aa | 394 | MALPVTALLLPLALLLHAARPQVNLRESGPALVKPTQTLTLTCTFSGF |
| Full CAR | | SLRTSGMCVSWIRQPPGKALEWLARIDWDEDKFYSTSLKTRLTISKDT |
| | | SDNQVVLRMTNMDPADTATYYCARSGAGGTSATAFDIWGPGTMVTVSS |
| | | GGGGSGGGGSGGGGSDIQMTQSPSSLSASVGDRVTITCRASQDIYNNL |
| | | AWFQLKPGSAPRSLMYAANKSQSGVPSRFSGSASGTDFTLTISSLQPE |
| | | DFATYYCQHYYRFPYSFGQGTKLEIKTTTPAPRPPTPAPTIASQPLSL |
| | | RPEACRPAAGGAVHTRGLDFACDIYIWAPLAGTCGVLLLSLVITLYCK |
| | | RGRKKLLYIFKQPFMRPVQTTQEEDGCSCRFPEEEEGGCELRVKFSRS |
| | | ADAPAYKQGQNQLYNELNLGRREEYDVLDKRRGRDPEMGGKPRRKNPQ |
| | | EGLYNELQKDKMAEAYSEIGMKGERRRGKGHDGLYQGLSTATKDTYDA |
| | | LHMQALPPR |
| 149363-nt | 395 | ATGGCCCTCCCTGTCACCGCCCTGCTGCTTCCGCTGGCTCTTCTGCTC |
| Full CAR | | CACGCCGCTCGGCCCCAAGTCAATCTGCGCGAATCCGGCCCCGCCTTG |
| | | GTCAAGCCTACCCAGACCCTCACTCTGACCTGTACTTTCTCCGGCTTC |
| | | TCCCTGCGGACTTCCGGGATGTGCGTGTCCTGGATCAGACAGCCTCCG |
| | | GGAAAGGCCCTGGAGTGGCTCGCTCGCATTGACTGGGATGAGGACAAG |
| | | TTCTACTCCACCTCACTCAAGACCAGGCTGACCATCAGCAAAGATACC |
| | | TCTGACAACCAAGTGGTGCTCCGCATGACCAACATGGACCCAGCCGAC |
| | | ACTGCCACTTACTACTGCGCGAGGAGCGGAGCGGGCGGAACCTCCGCC |
| | | ACCGCCTTCGATATTTGGGGCCCGGGTACCATGGTCACCGTGTCAAGC |
| | | GGAGGAGGGGGGTCCGGGGGCGGCGGTTCCGGGGGAGGCGGATCGGAC |
| | | ATTCAGATGACTCAGTCACCATCGTCCCTGAGCGCTAGCGTGGGCGAC |
| | | AGAGTGACAATCACTTGCCGGGCATCCCAGGACATCTATAACAACCTT |
| | | GCGTGGTTCCAGCTGAAGCCTGGTTCCGCACCGCGGTCACTTATGTAC |
| | | GCCGCCAACAAGAGCCAGTCGGGAGTGCCGTCCCGGTTTTCCGGTTCG |
| | | GCCTCGGGAACTGACTTCACCCTGACGATCTCCAGCCTGCAACCCGAG |
| | | GATTTCGCCACCTACTACTGCCAGCACTACTACCGCTTTCCCTACTCG |
| | | TTCGGACAGGGAACCAAGCTGGAAATCAAGACCACTACCCCAGCACCG |
| | | AGGCCACCCACCCCGGCTCCTACCATCGCCTCCCAGCCTCTGTCCCTG |
| | | CGTCCGGAGGCATGTAGACCCGCAGCTGGTGGGGCCGTGCATACCCGG |
| | | GGTCTTGACTTCGCCTGCGATATCTACATTTGGGCCCCTCTGGCTGGT |
| | | ACTTGCGGGGTCCTGCTGCTTTCACTCGTGATCACTCTTTACTGTAAG |
| | | CGCGGTCGGAAGAAGCTGCTGTACATCTTTAAGCAACCCTTCATGAGG |
| | | CCTGTGCAGACTACTCAAGAGGAGGACGGCTGTTCATGCCGGTTCCCA |
| | | GAGGAGGAGGAAGGCGGCTGCGAACTGCGCGTGAAATTCAGCCGCAGC |
| | | GCAGATGCTCCAGCCTACAAGCAGGGGCAGAACCAGCTCTACAACGAA |
| | | CTCAATCTTGGTCGGAGAGAGGAGTACGACGTGCTGGACAAGCGGAGA |
| | | GGACGGGACCCAGAAATGGGCGGGAAGCCGCGCAGAAAGAATCCCCAA |
| | | GAGGGCCTGTACAACGAGCTCCAAAAGGATAAGATGGCAGAAGCCTAT |
| | | AGCGAGATTGGTATGAAAGGGGAACGCAGAAGAGGCAAAGGCCACGAC |
| | | GGACTGTACCAGGGACTCAGCACCGCCACCAAGGACACCTATGACGCT |
| | | CTTCACATGCAGGCCCTGCCGCCTCGG |

149364

| | | |
|---|---|---|
| 149364-aa | 396 | EVQLVESGGGLVKPGGSLRLSCAASGFTFSSYSMNWVRQAPGKGLEWV |
| ScFv | | SSISSSSSYIYYADSVKGRFTISRDNAKNSLYLQMNSLRAEDTAVYYC |
| domain | | AKTIAAVYAFDIWGQGTTVTVSSGGGGSGGGGSGGGGSEIVLTQSPLS |
| | | LPVTPEEPASISCRSSQSLLHSNGYNYLDWYLQKPGQSPQLLIYLGSN |
| | | RASGVPDRFSGSGSGTDFTLKISRVEAEDVGVYYCMQALQTPYTFGQG |
| | | TKLEIK |
| 149364-nt | 397 | GAAGTGCAGCTTGTCGAATCCGGGGGGGGACTGGTCAAGCCGGGCGGA |
| ScFv | | TCACTGAGACTGTCCTGCGCCGCGAGCGGCTTCACGTTCTCCTCCTAC |
| domain | | TCCATGAACTGGGTCCGCCAAGCCCCCGGGAAGGGACTGGAATGGGTG |
| | | TCCTCTATCTCCTCGTCGTCGTCCTACATCTACTACGCCGACTCCGTG |
| | | AAGGGAAGATTCACCATTTCCCGCGACAACGCAAAGAACTCACTGTAC |
| | | TTGCAAATGAACTCACTCCGGGCCGAAGATACTGCTGTGTACTATTGC |
| | | GCCAAGACTATTGCCGCCGTCTACGCTTTCGACATCTGGGGCCAGGGA |
| | | ACCACCGTGACTGTGTCGTCCGGTGGTGGTGGCTCGGGCGGAGGAGGA |
| | | AGCGGCGGCGGGGGGTCCGAGATTGTGCTGACCCAGTCGCCACTGAGC |
| | | CTCCCTGTGACCCCCGAGGAACCCGCCAGCATCAGCTGCCGGTCCAGC |
| | | CAGTCCCTGCTCCACTCCAACGGATACAATTACCTCGATTGGTACCTT |
| | | CAGAAGCCTGGACAAAGCCCGCAGCTGCTCATCTACTTGGGATCAAAC |
| | | CGCGCGTCAGGAGTGCCTGACCGGTTCTCCGGCTCGGGCAGCGGTACC |
| | | GATTTCACCCTGAAAATCTCCAGGGTGGAGGCAGAGGACGTGGGAGTG |
| | | TATTACTGTATGCAGGCGCTGCAGACTCCGTACACATTTGGGCAGGGC |
| | | ACCAAGCTGGAGATCAAG |
| 149364-aa | 398 | EVQLVESGGGLVKPGGSLRLSCAASGFTFSSYSMNWVRQAPGKGLEWV |
| VH | | SSISSSSSYIYYADSVKGRFTISRDNAKNSLYLQMNSLRAEDTAVYYC |
| | | AKTIAAVYAFDIWGQGTTVTVSS |
| 149364-aa | 399 | EIVLTQSPLSLPVTPEEPASISCRSSQSLLHSNGYNYLDWYLQKPGQS |
| VL | | PQLLIYLGSNRASGVPDRFSGSGSGTDFTLKISRVEAEDVGVYYCMQA |
| | | LQTPYTFGQGTKLEIK |
| 149364-aa | 400 | MALPVTALLLPLALLLHAARPEVQLVESGGGLVKPGGSLRLSCAASGF |
| Full CAR | | TFSSYSMNWVRQAPGKGLEWVSSISSSSSYIYYADSVKGRFTISRDNA |
| | | KNSLYLQMNSLRAEDTAVYYCAKTIAAVYAFDIWGQGTTVTVSSGGGG |
| | | SGGGGSGGGGSEIVLTQSPLSLPVTPEEPASISCRSSQSLLHSNGYNY |
| | | LDWYLQKPGQSPQLLIYLGSNRASGVPDRFSGSGSGTDFTLKISRVEA |
| | | EDVGVYYCMQALQTPYTFGQGTKLEIKTTTPAPRPPTPAPTIASQPLS |
| | | LRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSR |
| | | SADAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDTYD |
| | | ALHMQALPPR |
| 149364-nt | 401 | ATGGCCCTCCCTGTCACCGCCCTGCTGCTTCCGCTGGCTCTTCTGCTC |
| Full CAR | | CACGCCGCTCGGCCCGAAGTGCAGCTTGTCGAATCCGGGGGGGGACTG |
| | | GTCAAGCCGGGCGGATCACTGAGACTGTCCTGCGCCGCGAGCGGCTTC |
| | | ACGTTCTCCTCCTACTCCATGAACTGGGTCCGCCAAGCCCCCGGGAAG |
| | | GGACTGGAATGGGTGTCCTCTATCTCCTCGTCGTCGTCCTACATCTAC |
| | | TACGCCGACTCCGTGAAGGGAAGATTCACCATTTCCCGCGACAACGCA |
| | | AAGAACTCACTGTACTTGCAAATGAACTCACTCCGGGCCGAAGATACT |
| | | GCTGTGTACTATTGCGCCAAGACTATTGCCGCCGTCTACGCTTTCGAC |
| | | ATCTGGGGCCAGGGAACCACCGTGACTGTGTCGTCCGGTGGTGGTGGC |
| | | TCGGGCGGAGGAGGAAGCGGCGGCGGGGGGTCCGAGATTGTGCTGACC |
| | | CAGTCGCCACTGAGCCTCCCTGTGACCCCCGAGGAACCCGCCAGCATC |
| | | AGCTGCCGGTCCAGCCAGTCCCTGCTCCACTCCAACGGATACAATTAC |
| | | CTCGATTGGTACCTTCAGAAGCCTGGACAAAGCCCGCAGCTGCTCATC |
| | | TACTTGGGATCAAACCGCGCGTCAGGAGTGCCTGACCGGTTCTCCGGC |
| | | TCGGGCAGCGGTACCGATTTCACCCTGAAAATCTCCAGGGTGGAGGCA |
| | | GAGGACGTGGGAGTGTATTACTGTATGCAGGCGCTGCAGACTCCGTAC |
| | | ACATTTGGGCAGGGCACCAAGCTGGAGATCAAGACCACTACCCCAGCA |
| | | CCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACC |
| | | CGGGGTCTTGACTTCGCCTGCGATATCTACATTTGGGCCCCTCTGGCT |
| | | GGTACTTGCGGGGTCCTGCTGCTTTCACTCGTGATCACTCTTTACTGT |
| | | AAGCGCGGTCGGAAGAAGCTGCTGTACATCTTTAAGCAACCCTTCATG |
| | | AGGCCTGTGCAGACTACTCAAGAGGAGGACGGCTGTTCATGCCGGTTC |
| | | CCAGAGGAGGAGGAAGGCGGCTGCGAACTGCGCGTGAAATTCAGCCGC |
| | | AGCGCAGATGCTCCAGCCTACAAGCAGGGGCAGAACCAGCTCTACAAC |
| | | GAACTCAATCTTGGTCGGAGAGAGGAGTACGACGTGCTGGACAAGCGG |
| | | AGAGGACGGGACCCAGAAATGGGCGGGAAGCCGCGCAGAAAGAATCCC |
| | | CAAGAGGGCCTGTACAACGAGCTCCAAAAGGATAAGATGGCAGAAGCC |
| | | TATAGCGAGATTGGTATGAAAGGGGAACGCAGAAGAGGCAAAGGCCAC |
| | | GACGGACTGTACCAGGGACTCAGCACCGCCACCAAGGACACCTATGAC |
| | | GCTCTTCACATGCAGGCCCTGCCGCCTCGG |

149365

| | | |
|---|---|---|
| 149365-aa | 402 | EVQLVESGGGLVKPGGSLRLSCAASGFTFSDYYMSWIRQAPGKGLEWV |
| ScFv | | SYISSSGSTIYYADSVKGRFTISRDNAKNSLYLQMNSLRAEDTAVYYC |
| domain | | ARDLRGAFDIWGQGTMVTVSSGGGGSGGGGSGGGGSSYVLTQSPSVSA |
| | | APGYTATISCGGNNIGTKSVHWYQQKPGQAPLLVIRDDSVRPSKIPGR |
| | | FSGSNSGNMATLTISGVQAGDEADFYCQVWDSDSEHVVFGGGTKLTVL |
| 149365-nt | 403 | GAAGTCCAGCTCGTGGAGTCCGGCGGAGGCCTTGTGAAGCCTGGAGGT |
| ScFv | | TCGCTGAGACTGTCCTGCGCCGCCTCCGGCTTCACCTTCTCCGACTAC |
| domain | | TACATGTCCTGGATCAGACAGGCCCCGGGAAAGGGCCTGGAATGGGTG |
| | | TCCTACATCTCGTCATCGGGCAGCACTATCTACTACGCGGACTCAGTG |
| | | AAGGGGCGGTTCACCATTTCCCGGGATAACGCGAAGAACTCGCTGTAT |
| | | CTGCAAATGAACTCACTGAGGGCCGAGGACACCGCCGTGTACTACTGC |
| | | GCCCGCGATCTCCGCGGGGCATTTGACATCTGGGGACAGGGAACCATG |
| | | GTCACAGTGTCCAGCGGAGGGGGAGGATCGGGTGGCGGAGGTTCCGGG |
| | | GGTGGAGGCTCCTCCTACGTGCTGACTCAGAGCCCAAGCGTCAGCGCT |
| | | GCGCCCGGTTACACGGCAACCATCTCCTGTGGCGGAAACAACATTGGG |
| | | ACCAAGTCTGTGCACTGGTATCAGCAGAAGCCGGGCCAAGCTCCCCTG |
| | | TTGGTGATCCGCGATGACTCCGTGCGGCCTAGCAAAATTCCGGGACGG |
| | | TTCTCCGGCTCCAACAGCGGCAATATGGCCACTCTCACCATCTCGGGA |
| | | GTGCAGGCCGGAGATGAAGCCGACTTCTACTGCCAAGTCTGGGACTCA |
| | | GACTCCGAGCATGTGGTGTTCGGGGGCGGAACCAAGCTGACTGTGCTC |
| 149365-aa | 404 | EVQLVESGGGLVKPGGSLRLSCAASGFTFSDYYMSWIRQAPGKGLEWV |
| VH | | SYISSSGSTIYYADSVKGRFTISRDNAKNSLYLQMNSLRAEDTAVYYC |
| | | ARDLRGAFDIWGQGTMVTVSS |
| 149365-aa | 405 | SYVLTQSPSVSAAPGYTATISCGGNNIGTKSVHWYQQKPGQAPLLVIR |
| VL | | DDSVRPSKIPGRFSGSNSGNMATLTISGVQAGDEADFYCQVWDSDSEH |
| | | VVFGGGTKLTVL |
| 149365-aa | 406 | MALPVTALLLPLALLLHAARPEVQLVESGGGLVKPGGSLRLSCAASGF |
| Full CAR | | TFSDYYMSWIRQAPGKGLEWVSYISSSGSTIYYADSVKGRFTISRDNA |
| | | KNSLYLQMNSLRAEDTAVYYCARDLRGAFDIWGQGTMVTVSSGGGGSG |
| | | GGGSGGGGSSYVLTQSPSVSAAPGYTATISCGGNNIGTKSVHWYQQKP |
| | | GQAPLLVIRDDSVRPSKIPGRFSGSNSGNMATLTISGVQAGDEADFYC |
| | | QVWDSDSEHVVFGGGTKLTVLTTTPAPRPPTPAPTIASQPLSLRPEAC |
| | | RPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPA |
| | | YKQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQA |
| | | LPPR |
| 149365-nt | 407 | ATGGCCCTCCCTGTCACCGCCCTGCTGCTTCCGCTGGCTCTTCTGCTC |
| Full CAR | | CACGCCGCTCGGCCCGAAGTCCAGCTCGTGGAGTCCGGCGGAGGCCTT |
| | | GTGAAGCCTGGAGGTTCGCTGAGACTGTCCTGCGCCGCCTCCGGCTTC |
| | | ACCTTCTCCGACTACTACATGTCCTGGATCAGACAGGCCCCGGGAAAG |
| | | GGCCTGGAATGGGTGTCCTACATCTCGTCATCGGGCAGCACTATCTAC |
| | | TACGCGGACTCAGTGAAGGGGCGGTTCACCATTTCCCGGGATAACGCG |
| | | AAGAACTCGCTGTATCTGCAAATGAACTCACTGAGGGCCGAGGACACC |
| | | GCCGTGTACTACTGCGCCCGCGATCTCCGCGGGGCATTTGACATCTGG |
| | | GGACAGGGAACCATGGTCACAGTGTCCAGCGGAGGGGGAGGATCGGGT |
| | | GGCGGAGGTTCCGGGGGTGGAGGCTCCTCCTACGTGCTGACTCAGAGC |
| | | CCAAGCGTCAGCGCTGCGCCCGGTTACACGGCAACCATCTCCTGTGGC |
| | | GGAAACAACATTGGGACCAAGTCTGTGCACTGGTATCAGCAGAAGCCG |
| | | GGCCAAGCTCCCCTGTTGGTGATCCGCGATGACTCCGTGCGGCCTAGC |
| | | AAAATTCCGGGACGGTTCTCCGGCTCCAACAGCGGCAATATGGCCACT |
| | | CTCACCATCTCGGGAGTGCAGGCCGGAGATGAAGCCGACTTCTACTGC |
| | | CAAGTCTGGGACTCAGACTCCGAGCATGTGGTGTTCGGGGGCGGAACC |
| | | AAGCTGACTGTGCTCACCACTACCCCAGCACCGAGGCCACCCACCCCG |
| | | GCTCCTACCATCGCCTCCCAGCCTCTGTCCCTGCGTCCGGAGGCATGT |
| | | AGACCCGCAGCTGGTGGGGCCGTGCATACCCGGGGTCTTGACTTCGCC |
| | | TGCGATATCTACATTTGGGCCCCTCTGGCTGGTACTTGCGGGGTCCTG |
| | | CTGCTTTCACTCGTGATCACTCTTTACTGTAAGCGCGGTCGGAAGAAG |
| | | CTGCTGTACATCTTTAAGCAACCCTTCATGAGGCCTGTGCAGACTACT |
| | | CAAGAGGAGGACGGCTGTTCATGCCGGTTCCCAGAGGAGGAGGAAGGC |
| | | GGCTGCGAACTGCGCGTGAAATTCAGCCGCAGCGCAGATGCTCCAGCC |
| | | TACAAGCAGGGGCAGAACCAGCTCTACAACGAACTCAATCTTGGTCGG |
| | | AGAGAGGAGTACGACGTGCTGGACAAGCGGAGAGGACGGGACCCAGAA |
| | | ATGGGCGGGAAGCCGCGCAGAAAGAATCCCCAAGAGGGCCTGTACAAC |
| | | GAGCTCCAAAAGGATAAGATGGCAGAAGCCTATAGCGAGATTGGTATG |
| | | AAAGGGGAACGCAGAAGAGGCAAAGGCCACGACGGACTGTACCAGGGA |
| | | CTCAGCACCGCCACCAAGGACACCTATGACGCTCTTCACATGCAGGCC |
| | | CTGCCGCCTCGG |

149366

| | | |
|---|---|---|
| 149366-aa | 408 | QVQLVQSGAEVKKPGASVKVSCKPSGYTVTSHYIHWVRRAPGQGLEWM |
| ScFv | | GMINPSGGVTAYSQTLQGRVTMTSDTSSSTVYMELSSLRSEDTAMYYC |
| domain | | AREGSGSGWYFDFWGRGTLVTVSSGGGGSGGGGSGGGGSSYVLTQPPS |
| | | VSVSPGQTASITCSGDGLSKKYVSWYQQKAGQSPVVLISRDKERPSGI |
| | | PDRFSGSNSADTATLTISGTQAMDEADYYCQAWDDTTVVFGGGTKLTV |
| | | L |
| 149366-nt | 409 | CAAGTGCAGCTGGTGCAGAGCGGGGCCGAAGTCAAGAAGCCGGGAGCC |
| ScFv | | TCCGTGAAAGTGTCCTGCAAGCCTTCGGGATACACCGTGACCTCCCAC |
| domain | | TACATTCATTGGGTCCGCCGCGCCCCCGGCCAAGGACTCGAGTGGATG |
| | | GGCATGATCAACCCTAGCGGCGGAGTGACCGCGTACAGCCAGACGCTG |
| | | CAGGGACGCGTGACTATGACCTCGGATACCTCCTCCTCCACCGTCTAT |
| | | ATGGAACTGTCCAGCCTGCGGTCCGAGGATACCGCCATGTACTACTGC |
| | | GCCCGGGAAGGATCAGGCTCCGGGTGGTATTTCGACTTCTGGGGAAGA |
| | | GGCACCCTCGTGACTGTGTCATCTGGGGGAGGGGGTTCCGGTGGTGGC |
| | | GGATCGGGAGGAGGCGGTTCATCCTACGTGCTGACCCAGCCACCCTCC |
| | | GTGTCCGTGAGCCCCGGCCAGACTGCATCGATTACATGTAGCGGCGAC |
| | | GGCCTCTCCAAGAAATACGTGTCGTGGTACCAGCAGAAGGCCGGACAG |
| | | AGCCCGGTGGTGCTGATCTCAAGAGATAAGGAGCGGCCTAGCGGAATC |
| | | CCGGACAGGTTCTCGGGTTCCAACTCCGCGGACACTGCTACTCTGACC |
| | | ATCTCGGGGACCCAGGCTATGGACGAAGCCGATTACTACTGCCAAGCC |
| | | TGGGACGACACTACTGTCGTGTTTGGAGGGGGCACCAAGTTGACCGTC |
| | | CTT |
| 149366-aa | 410 | QVQLVQSGAEVKKPGASVKVSCKPSGYTVTSHYIHWVRRAPGQGLEWM |
| VH | | GMINPSGGVTAYSQTLQGRVTMTSDTSSSTVYMELSSLRSEDTAMYYC |
| | | AREGSGSGWYFDFWGRGTLVTVSS |
| 149366-aa | 411 | SYVLTQPPSVSVSPGQTASITCSGDGLSKKYVSWYQQKAGQSPVVLIS |
| VL | | RDKERPSGIPDRFSGSNSADTATLTISGTQAMDEADYYCQAWDDTTVV |
| | | FGGGTKLTVL |
| 149366-aa | 412 | MALPVTALLLPLALLLHAARPQVQLVQSGAEVKKPGASVKVSCKPSGY |
| Full CAR | | TVTSHYIHWVRRAPGQGLEWMGMINPSGGVTAYSQTLQGRVTMTSDTS |
| | | SSTVYMELSSLRSEDTAMYYCAREGSGSGWYFDFWGRGTLVTVSSGGG |
| | | GSGGGGSGGGGSSYVLTQPPSVSVSPGQTASITCSGDGLSKKYVSWYQ |
| | | QKAGQSPVVLISRDKERPSGIPDRFSGSNSADTATLTISGTQAMDEAD |
| | | YYCQAWDDTTVVFGGGTKLTVLTTTPAPRPPTPAPTIASQPLSLRPEA |
| | | CRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAP |
| | | AYKQGQNQLYNELNLGRREEYDVLDKRRGRDPEMGGKPRRKNPQEGLY |
| | | NELQKDKMAEAYSEIGMKGERRRGKGHDGLYQGLSTATKDTYDALHMQ |
| | | ALPPR |
| 149366-nt | 413 | ATGGCCCTCCCTGTCACCGCCCTGCTGCTTCCGCTGGCTCTTCTGCTC |
| Full CAR | | CACGCCGCTCGGCCCCAAGTGCAGCTGGTGCAGAGCGGGGCCGAAGTC |
| | | AAGAAGCCGGGAGCCTCCGTGAAAGTGTCCTGCAAGCCTTCGGGATAC |
| | | ACCGTGACCTCCCACTACATTCATTGGGTCCGCCGCGCCCCCGGCCAA |
| | | GGACTCGAGTGGATGGGCATGATCAACCCTAGCGGCGGAGTGACCGCG |
| | | TACAGCCAGACGCTGCAGGGACGCGTGACTATGACCTCGGATACCTCC |
| | | TCCTCCACCGTCTATATGGAACTGTCCAGCCTGCGGTCCGAGGATACC |
| | | GCCATGTACTACTGCGCCCGGGAAGGATCAGGCTCCGGGTGGTATTTC |
| | | GACTTCTGGGGAAGAGGCACCCTCGTGACTGTGTCATCTGGGGGAGGG |
| | | GGTTCCGGTGGTGGCGGATCGGGAGGAGGCGGTTCATCCTACGTGCTG |
| | | ACCCAGCCACCCTCCGTGTCCGTGAGCCCCGGCCAGACTGCATCGATT |
| | | ACATGTAGCGGCGACGGCCTCTCCAAGAAATACGTGTCGTGGTACCAG |
| | | CAGAAGGCCGGACAGAGCCCGGTGGTGCTGATCTCAAGAGATAAGGAG |
| | | CGGCCTAGCGGAATCCCGGACAGGTTCTCGGGTTCCAACTCCGCGGAC |
| | | ACTGCTACTCTGACCATCTCGGGGACCCAGGCTATGGACGAAGCCGAT |
| | | TACTACTGCCAAGCCTGGGACGACACTACTGTCGTGTTTGGAGGGGGC |
| | | ACCAAGTTGACCGTCCTTACCACTACCCCAGCACCGAGGCCACCCACC |
| | | CCGGCTCCTACCATCGCCTCCCAGCCTCTGTCCCTGCGTCCGGAGGCA |
| | | TGTAGACCCGCAGCTGGTGGGGCCGTGCATACCCGGGGTCTTGACTTC |
| | | GCCTGCGATATCTACATTTGGGCCCCTCTGGCTGGTACTTGCGGGGTC |
| | | CTGCTGCTTTCACTCGTGATCACTCTTTACTGTAAGCGCGGTCGGAAG |
| | | AAGCTGCTGTACATCTTTAAGCAACCCTTCATGAGGCCTGTGCAGACT |
| | | ACTCAAGAGGAGGACGGCTGTTCATGCCGGTTCCCAGAGGAGGAGGAA |
| | | GGCGGCTGCGAACTGCGCGTGAAATTCAGCCGCAGCGCAGATGCTCCA |
| | | GCCTACAAGCAGGGGCAGAACCAGCTCTACAACGAACTCAATCTTGGT |
| | | CGGAGAGAGGAGTACGACGTGCTGGACAAGCGGAGAGGACGGGACCCA |
| | | GAAATGGGCGGGAAGCCGCGCAGAAAGAATCCCCAAGAGGGCCTGTAC |
| | | AACGAGCTCCAAAAGGATAAGATGGCAGAAGCCTATAGCGAGATTGGT |
| | | ATGAAAGGGGAACGCAGAAGAGGCAAAGGCCACGACGGACTGTACCAG |
| | | GGACTCAGCACCGCCACCAAGGACACCTATGACGCTCTTCACATGCAG |
| | | GCCCTGCCGCCTCGG |

149367

| | | |
|---|---|---|
| 149367-aa | 414 | QVQLQESGPGLVKPSQTLSLTCTVSGGSISSGGYYWSWIRQHPGKGLE |
| ScFv | | WIGYIYYSGSTYYNPSLKSRVTISVDTSKNQFSLKLSSVTAADTAVYY |
| domain | | CARAGIAARLRGAFDIWGQGTMVTVSSGGGGSGGGGSGGGGSDIVMTQ |
| | | SPSSVSASVGDRVIITCRASQGIRNWLAWYQQKPGKAPNLLIYAASNL |
| | | QSGVPSRFSGSGSGADFTLTISSLQPEDVATYYCQKYNSAPFTFGPGT |
| | | KVDIK |
| 149367-nt | 415 | CAAGTGCAGCTTCAGGAGAGCGGCCCGGGACTCGTGAAGCCGTCCCAG |
| ScFv | | ACCCTGTCCCTGACTTGCACCGTGTCGGGAGGAAGCATCTCGAGCGGA |
| domain | | GGCTACTATTGGTCGTGGATTCGGCAGCACCCTGGAAAGGGCCTGGAA |
| | | TGGATCGGCTACATCTACTACTCCGGCTCGACCTACTACAACCCATCG |
| | | CTGAAGTCCAGAGTGACAATCTCAGTGGACACGTCCAAGAATCAGTTC |
| | | AGCCTGAAGCTCTCTTCCGTGACTGCGGCCGACACCGCCGTGTACTAC |
| | | TGCGCACGCGCTGGAATTGCCGCCCGGCTGAGGGGTGCCTTCGACATT |
| | | TGGGGACAGGGCACCATGGTCACCGTGTCCTCCGGCGGCGGAGGTTCC |
| | | GGGGGTGGAGGCTCAGGAGGAGGGGGGTCCGACATCGTCATGACTCAG |
| | | TCGCCCTCAAGCGTCAGCGCGTCCGTCGGGGACAGAGTGATCATCACC |
| | | TGTCGGGCGTCCCAGGGAATTCGCAACTGGCTGGCCTGGTATCAGCAG |
| | | AAGCCCGGAAAGGCCCCCAACCTGTTGATCTACGCCGCCTCAAACCTC |
| | | CAATCCGGGGTGCCGAGCCGCTTCAGCGGCTCCGGTTCGGGTGCCGAT |
| | | TTCACTCTGACCATCTCCTCCCTGCAACCTGAAGATGTGGCTACCTAC |
| | | TACTGCCAAAAGTACAACTCCGCACCTTTTACTTTCGGACCGGGGACC |
| | | AAAGTGGACATTAAG |
| 149367-aa | 416 | QVQLQESGPGLVKPSQTLSLTCTVSGGSISSGGYYWSWIRQHPGKGLE |
| VH | | WIGYIYYSGSTYYNPSLKSRVTISVDTSKNQFSLKLSSVTAADTAVYY |
| | | CARAGIAARLRGAFDIWGQGTMVTVSS |
| 149367-aa | 417 | DIVMTQSPSSVSASVGDRVIITCRASQGIRNWLAWYQQKPGKAPNLLI |
| VL | | YAASNLQSGVPSRFSGSGSGADFTLTISSLQPEDVATYYCQKYNSAPF |
| | | TFGPGTKVDIK |
| 149367-aa | 418 | MALPVTALLLPLALLLHAARPQVQLQESGPGLVKPSQTLSLTCTVSGG |
| Full CAR | | SISSGGYYWSWIRQHPGKGLEWIGYIYYSGSTYYNPSLKSRVTISVDT |
| | | SKNQFSLKLSSVTAADTAVYYCARAGIAARLRGAFDIWGQGTMVTVSS |
| | | GGGGSGGGGSGGGGSDIVMTQSPSSVSASVGDRVIITCRASQGIRNWL |
| | | AWYQQKPGKAPNLLIYAASNLQSGVPSRFSGSGSGADFTLTISSLQPE |
| | | DVATYYCQKYNSAPFTFGPGTKVDIKTTTPAPRPPTPAPTIASQPLSL |
| | | RPEACRPAAGGAVHTRGLDFACDIYIWAPLAGTCGVLLLSLVITLYCK |
| | | RGRKKLLYIFKQPFMRPVQTTQEEDGCSCRFPEEEEGGCELRVKFSRS |
| | | ADAPAYKQGQNQLYNELNLGRREEYDVLDKRRGRDPEMGGKPRRKNPQ |
| | | EGLYNELQKDKMAEAYSEIGMKGERRRGKGHDGLYQGLSTATKDTYDA |
| | | LHMQALPPR |
| 149367-nt | 419 | ATGGCCCTCCCTGTCACCGCCCTGCTGCTTCCGCTGGCTCTTCTGCTC |
| Full CAR | | CACGCCGCTCGGCCCCAAGTGCAGCTTCAGGAGAGCGGCCCGGGACTC |
| | | GTGAAGCCGTCCCAGACCCTGTCCCTGACTTGCACCGTGTCGGGAGGA |
| | | AGCATCTCGAGCGGAGGCTACTATTGGTCGTGGATTCGGCAGCACCCT |
| | | GGAAAGGGCCTGGAATGGATCGGCTACATCTACTACTCCGGCTCGACC |
| | | TACTACAACCCATCGCTGAAGTCCAGAGTGACAATCTCAGTGGACACG |
| | | TCCAAGAATCAGTTCAGCCTGAAGCTCTCTTCCGTGACTGCGGCCGAC |
| | | ACCGCCGTGTACTACTGCGCACGCGCTGGAATTGCCGCCCGGCTGAGG |
| | | GGTGCCTTCGACATTTGGGGACAGGGCACCATGGTCACCGTGTCCTCC |
| | | GGCGGCGGAGGTTCCGGGGGTGGAGGCTCAGGAGGAGGGGGGTCCGAC |
| | | ATCGTCATGACTCAGTCGCCCTCAAGCGTCAGCGCGTCCGTCGGGGAC |
| | | AGAGTGATCATCACCTGTCGGGCGTCCCAGGGAATTCGCAACTGGCTG |
| | | GCCTGGTATCAGCAGAAGCCCGGAAAGGCCCCCAACCTGTTGATCTAC |
| | | GCCGCCTCAAACCTCCAATCCGGGGTGCCGAGCCGCTTCAGCGGCTCC |
| | | GGTTCGGGTGCCGATTTCACTCTGACCATCTCCTCCCTGCAACCTGAA |
| | | GATGTGGCTACCTACTACTGCCAAAAGTACAACTCCGCACCTTTTACT |
| | | TTCGGACCGGGGACCAAAGTGGACATTAAGACCACTACCCCAGCACCG |
| | | AGGCCACCCACCCCGGCTCCTACCATCGCCTCCCAGCCTCTGTCCCTG |
| | | CGTCCGGAGGCATGTAGACCCGCAGCTGGTGGGGCCGTGCATACCCGG |
| | | GGTCTTGACTTCGCCTGCGATATCTACATTTGGGCCCCTCTGGCTGGT |
| | | ACTTGCGGGGTCCTGCTGCTTTCACTCGTGATCACTCTTTACTGTAAG |
| | | CGCGGTCGGAAGAAGCTGCTGTACATCTTTAAGCAACCCTTCATGAGG |
| | | CCTGTGCAGACTACTCAAGAGGAGGACGGCTGTTCATGCCGGTTCCCA |
| | | GAGGAGGAGGAAGGCGGCTGCGAACTGCGCGTGAAATTCAGCCGCAGC |
| | | GCAGATGCTCCAGCCTACAAGCAGGGGCAGAACCAGCTCTACAACGAA |
| | | CTCAATCTTGGTCGGAGAGAGGAGTACGACGTGCTGGACAAGCGGAGA |
| | | GGACGGGACCCAGAAATGGGCGGGAAGCCGCGCAGAAAGAATCCCCAA |
| | | GAGGGCCTGTACAACGAGCTCCAAAAGGATAAGATGGCAGAAGCCTAT |
| | | AGCGAGATTGGTATGAAAGGGGAACGCAGAAGAGGCAAAGGCCACGAC |
| | | GGACTGTACCAGGGACTCAGCACCGCCACCAAGGACACCTATGACGCT |
| | | CTTCACATGCAGGCCCTGCCGCCTCGG |

149368

| | | |
|---|---|---|
| 149368-aa | 420 | QVQLVQSGAEVKKPGSSVKVSCKASGGTFSSYAISWVRQAPGQGLEWM |
| ScFv | | GGIIPIFGTANYAQKFQGRVTITADESTSTAYMELSSLRSEDTAVYYC |
| domain | | ARRGGYQLLRWDVGLLRSAFDIWGQGTMVTVSSGGGGSGGGGSGGGGS |
| | | SYVLTQPPSVSVAPGQTARITCGGNNIGSKSVHWYQQKPGQAPVLVLY |
| | | GKNNRPSGVPDRFSGSRSGTTASLTITGAQAEDEADYYCSSRDSSGDH |
| | | LRVFGTGTKVTVL |
| 149368-nt | 421 | CAAGTGCAGCTGGTCCAGTCGGGCGCCGAGGTCAAGAAGCCCGGGAGC |
| ScFv | | TCTGTGAAAGTGTCCTGCAAGGCCTCCGGGGGCACCTTTAGCTCCTAC |
| domain | | GCCATCTCCTGGGTCCGCCAAGCACCGGGTCAAGGCCTGGAGTGGATG |
| | | GGGGGAATTATCCCTATCTTCGGCACTGCCAACTACGCCCAGAAGTTC |
| | | CAGGGACGCGTGACCATTACCGCGGACGAATCCACCTCCACCGCTTAT |
| | | ATGGAGCTGTCCAGCTTGCGCTCGGAAGATACCGCCGTGTACTACTGC |
| | | GCCCGGAGGGGTGGATACCAGCTGCTGAGATGGGACGTGGGCCTCCTG |
| | | CGGTCGGCGTTCGACATCTGGGGCCAGGGCACTATGGTCACTGTGTCC |
| | | AGCGGAGGAGGCGGATCGGGAGGCGGCGGATCAGGGGGAGGCGGTTCC |
| | | AGCTACGTGCTTACTCAACCCCCTTCGGTGTCCGTGGCCCCGGGACAG |
| | | ACCGCCAGAATCACTTGCGGAGGAAACAACATTGGGTCCAAGAGCGTG |
| | | CATTGGTACCAGCAGAAGCCAGGACAGGCCCCTGTGCTGGTGCTCTAC |
| | | GGGAAGAACAATCGGCCCAGCGGAGTGCCGGACAGGTTCTCGGGTTCA |
| | | CGCTCCGGTACAACCGCTTCACTGACTATCACCGGGGCCCAGGCAGAG |
| | | GATGAAGCGGACTACTACTGTTCCTCCCGGGATTCATCCGGCGACCAC |
| | | CTCCGGGTGTTCGGAACCGGAACGAAGGTCACCGTGCTG |
| 149368-aa | 422 | QVQLVQSGAEVKKPGSSVKVSCKASGGTFSSYAISWVRQAPGQGLEWM |
| VH | | GGIIPIFGTANYAQKFQGRVTITADESTSTAYMELSSLRSEDTAVYYC |
| | | ARRGGYQLLRWDVGLLRSAFDIWGQGTMVTVSS |
| 149368-aa | 423 | SYVLTQPPSVSVAPGQTARITCGGNNIGSKSVHWYQQKPGQAPVLVLY |
| VL | | GKNNRPSGVPDRFSGSRSGTTASLTITGAQAEDEADYYCSSRDSSGDH |
| | | LRVFGTGTKVTVL |
| 149368-aa | 424 | MALPVTALLLPLALLLHAARPQVQLVQSGAEVKKPGSSVKVSCKASGG |
| Full CAR | | TESSYAISWVRQAPGQGLEWMGGIIPIFGTANYAQKFQGRVTITADES |
| | | TSTAYMELSSLRSEDTAVYYCARRGGYQLLRWDVGLLRSAFDIWGQGT |
| | | MVTVSSGGGGSGGGGSGGGGSSYVLTQPPSVSVAPGQTARITCGGNNI |
| | | GSKSVHWYQQKPGQAPVLVLYGKNNRPSGVPDRFSGSRSGTTASLTIT |
| | | GAQAEDEADYYCSSRDSSGDHLRVFGTGTKVTVLTTTPAPRPPTPAPT |
| | | IASQPLSLRPEACRPAAGGAVHTRGLDFACDIYIWAPLAGTCGVLLLS |
| | | LVITLYCKRGRKKLLYIFKQPFMRPVQTTQEEDGCSCRFPEEEEGGCE |
| | | LRVKFSRSADAPAYKQGQNQLYNELNLGRREEYDVLDKRRGRDPEMGG |
| | | KPRRKNPQEGLYNELQKDKMAEAYSEIGMKGERRRGKGHDGLYQGLST |
| | | ATKDTYDALHMQALPPR |
| 149368-nt | 425 | ATGGCCCTCCCTGTCACCGCCCTGCTGCTTCCGCTGGCTCTTCTGCTC |
| Full CAR | | CACGCCGCTCGGCCCCAAGTGCAGCTGGTCCAGTCGGGCGCCGAGGTC |
| | | AAGAAGCCCGGGAGCTCTGTGAAAGTGTCCTGCAAGGCCTCCGGGGGC |
| | | ACCTTTAGCTCCTACGCCATCTCCTGGGTCCGCCAAGCACCGGGTCAA |
| | | GGCCTGGAGTGGATGGGGGGAATTATCCCTATCTTCGGCACTGCCAAC |
| | | TACGCCCAGAAGTTCCAGGGACGCGTGACCATTACCGCGGACGAATCC |
| | | ACCTCCACCGCTTATATGGAGCTGTCCAGCTTGCGCTCGGAAGATACC |
| | | GCCGTGTACTACTGCGCCCGGAGGGGTGGATACCAGCTGCTGAGATGG |
| | | GACGTGGGCCTCCTGCGGTCGGCGTTCGACATCTGGGGCCAGGGCACT |
| | | ATGGTCACTGTGTCCAGCGGAGGAGGCGGATCGGGAGGCGGCGGATCA |
| | | GGGGGAGGCGGTTCCAGCTACGTGCTTACTCAACCCCCTTCGGTGTCC |
| | | GTGGCCCCGGGACAGACCGCCAGAATCACTTGCGGAGGAAACAACATT |
| | | GGGTCCAAGAGCGTGCATTGGTACCAGCAGAAGCCAGGACAGGCCCCT |
| | | GTGCTGGTGCTCTACGGGAAGAACAATCGGCCCAGCGGAGTGCCGGAC |
| | | AGGTTCTCGGGTTCACGCTCCGGTACAACCGCTTCACTGACTATCACC |
| | | GGGGCCCAGGCAGAGGATGAAGCGGACTACTACTGTTCCTCCCGGGAT |
| | | TCATCCGGCGACCACCTCCGGGTGTTCGGAACCGGAACGAAGGTCACC |
| | | GTGCTGACCACTACCCCAGCACCGAGGCCACCCACCCCGGCTCCTACC |
| | | ATCGCCTCCCAGCCTCTGTCCCTGCGTCCGGAGGCATGTAGACCCGCA |
| | | GCTGGTGGGGCCGTGCATACCCGGGGTCTTGACTTCGCCTGCGATATC |
| | | TACATTTGGGCCCCTCTGGCTGGTACTTGCGGGGTCCTGCTGCTTTCA |
| | | CTCGTGATCACTCTTTACTGTAAGCGCGGTCGGAAGAAGCTGCTGTAC |
| | | ATCTTTAAGCAACCCTTCATGAGGCCTGTGCAGACTACTCAAGAGGAG |
| | | GACGGCTGTTCATGCCGGTTCCCAGAGGAGGAGGAAGGCGGCTGCGAA |
| | | CTGCGCGTGAAATTCAGCCGCAGCGCAGATGCTCCAGCCTACAAGCAG |
| | | GGGCAGAACCAGCTCTACAACGAACTCAATCTTGGTCGGAGAGAGGAG |
| | | TACGACGTGCTGGACAAGCGGAGAGGACGGGACCCAGAAATGGGCGGG |
| | | AAGCCGCGCAGAAAGAATCCCCAAGAGGGCCTGTACAACGAGCTCCAA |
| | | AAGGATAAGATGGCAGAAGCCTATAGCGAGATTGGTATGAAAGGGGAA |
| | | CGCAGAAGAGGCAAAGGCCACGACGGACTGTACCAGGGACTCAGCACC |
| | | GCCACCAAGGACACCTATGACGCTCTTCACATGCAGGCCCTGCCGCCT |
| | | CGG |

149369

| | | |
|---|---|---|
| 149369-aa | 426 | EVQLQQSGPGLVKPSQTLSLTCAISGDSVSSNSAAWNWIRQSPSRGLE |
| ScFv | | WLGRTYYRSKWYSFYAISLKSRIIINPDTSKNQFSLQLKSVTPEDTAV |
| domain | | YYCARSSPEGLFLYWFDPWGQGTLVTVSSGGDGSGGGGSGGGGSSSEL |
| | | TQDPAVSVALGQTIRITCQGDSLGNYYATWYQQKPGQAPVLVIYGTNN |
| | | RPSGIPDRFSASSSGNTASLTITGAQAEDEADYYCNSRDSSGHHLLFG |
| | | TGTKVTVL |
| 149369-nt | 427 | GAAGTGCAGCTCCAACAGTCAGGACCGGGGCTCGTGAAGCCATCCCAG |
| ScFv | | ACCCTGTCCCTGACTTGTGCCATCTCGGGAGATAGCGTGTCATCGAAC |
| domain | | TCCGCCGCCTGGAACTGGATTCGGCAGAGCCCGTCCCGCGGACTGGAG |
| | | TGGCTTGGAAGGACCTACTACCGGTCCAAGTGGTACTCTTTCTACGCG |
| | | ATCTCGCTGAAGTCCCGCATTATCATTAACCCTGATACCTCCAAGAAT |
| | | CAGTTCTCCCTCCAACTGAAATCCGTCACCCCCGAGGACACAGCAGTG |
| | | TATTACTGCGCACGGAGCAGCCCCGAAGGACTGTTCCTGTATTGGTTT |
| | | GACCCCTGGGGCCAGGGGACTCTTGTGACCGTGTCGAGCGGCGGAGAT |
| | | GGGTCCGGTGGCGGTGGTTCGGGGGGCGGCGGATCATCATCCGAACTG |
| | | ACCCAGGACCCGGCTGTGTCCGTGGCGCTGGGACAAACCATCCGCATT |
| | | ACGTGCCAGGGAGACTCCCTGGGCAACTACTACGCCACTTGGTACCAG |
| | | CAGAAGCCGGGCCAAGCCCCTGTGTTGGTCATCTACGGGACCAACAAC |
| | | AGACCTTCCGGCATCCCCGACCGGTTCAGCGCTTCGTCCTCCGGCAAC |
| | | ACTGCCAGCCTGACCATCACTGGAGCGCAGGCCGAAGATGAGGCCGAC |
| | | TACTACTGCAACAGCAGAGACTCCTCGGGTCATCACCTCTTGTTCGGA |
| | | ACTGGAACCAAGGTCACCGTGCTG |
| 149369-aa | 428 | EVQLQQSGPGLVKPSQTLSLTCAISGDSVSSNSAAWNWIRQSPSRGLE |
| VH | | WLGRTYYRSKWYSFYAISLKSRIIINPDTSKNQFSLQLKSVTPEDTAV |
| | | YYCARSSPEGLFLYWFDPWGQGTLVTVSS |
| 149369-aa | 429 | SSELTQDPAVSVALGQTIRITCQGDSLGNYYATWYQQKPGQAPVLVIY |
| VL | | GTNNRPSGIPDRFSASSSGNTASLTITGAQAEDEADYYCNSRDSSGHH |
| | | LLFGTGTKVTVL |
| 149369-aa | 430 | MALPVTALLLPLALLLHAARPEVQLQQSGPGLVKPSQTLSLTCAISGD |
| Full CAR | | SVSSNSAAWNWIRQSPSRGLEWLGRTYYRSKWYSFYAISLKSRIIINP |
| | | DTSKNQFSLQLKSVTPEDTAVYYCARSSPEGLFLYWFDPWGQGTLVTV |
| | | SSGGDGSGGGGSGGGGSSSELTQDPAVSVALGQTIRITCQGDSLGNYY |
| | | ATWYQQKPGQAPVLVIYGTNNRPSGIPDRFSASSSGNTASLTITGAQA |
| | | EDEADYYCNSRDSSGHHLLFGTGTKVTVLTTTPAPRPPTPAPTIASQP |
| | | LSLRPEACRPAAGGAVHTRGLDFACDIYIWAPLAGTCGVLLLSLVITL |
| | | YCKRGRKKLLYIFKQPFMRPVQTTQEEDGCSCRFPEEEEGGCELRVKF |
| | | SRSADAPAYKQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPR |
| 149369-nt | 431 | ATGGCCCTCCCTGTCACCGCCCTGCTGCTTCCGCTGGCTCTTCTGCTC |
| Full CAR | | CACGCCGCTCGGCCCGAAGTGCAGCTCCAACAGTCAGGACCGGGGCTC |
| | | GTGAAGCCATCCCAGACCCTGTCCCTGACTTGTGCCATCTCGGGAGAT |
| | | AGCGTGTCATCGAACTCCGCCGCCTGGAACTGGATTCGGCAGAGCCCG |
| | | TCCCGCGGACTGGAGTGGCTTGGAAGGACCTACTACCGGTCCAAGTGG |
| | | TACTCTTTCTACGCGATCTCGCTGAAGTCCCGCATTATCATTAACCCT |
| | | GATACCTCCAAGAATCAGTTCTCCCTCCAACTGAAATCCGTCACCCCC |
| | | GAGGACACAGCAGTGTATTACTGCGCACGGAGCAGCCCCGAAGGACTG |
| | | TTCCTGTATTGGTTTGACCCCTGGGGCCAGGGGACTCTTGTGACCGTG |
| | | TCGAGCGGCGGAGATGGGTCCGGTGGCGGTGGTTCGGGGGGCGGCGGA |
| | | TCATCATCCGAACTGACCCAGGACCCGGCTGTGTCCGTGGCGCTGGGA |
| | | CAAACCATCCGCATTACGTGCCAGGGAGACTCCCTGGGCAACTACTAC |
| | | GCCACTTGGTACCAGCAGAAGCCGGGCCAAGCCCCTGTGTTGGTCATC |
| | | TACGGGACCAACAACAGACCTTCCGGCATCCCCGACCGGTTCAGCGCT |
| | | TCGTCCTCCGGCAACACTGCCAGCCTGACCATCACTGGAGCGCAGGCC |
| | | GAAGATGAGGCCGACTACTACTGCAACAGCAGAGACTCCTCGGGTCAT |
| | | CACCTCTTGTTCGGAACTGGAACCAAGGTCACCGTGCTGACCACTACC |
| | | CCAGCACCGAGGCCACCCACCCCGGCTCCTACCATCGCCTCCCAGCCT |
| | | CTGTCCCTGCGTCCGGAGGCATGTAGACCCGCAGCTGGTGGGGCCGTG |
| | | CATACCCGGGGTCTTGACTTCGCCTGCGATATCTACATTTGGGCCCCT |
| | | CTGGCTGGTACTTGCGGGGTCCTGCTGCTTTCACTCGTGATCACTCTT |
| | | TACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGC |
| | | CGGTTCCCAGAGGAGGAGGAAGGCGGCTGCGAACTGCGCGTGAAATTC |
| | | AGCCGCAGCGCAGATGCTCCAGCCTACAAGCAGGGGCAGAACCAGCTC |
| | | TACAACGAACTCAATCTTGGTCGGAGAGAGGAGTACGACGTGCTGGAC |
| | | AAGCGGAGAGGACGGGACCCAGAAATGGGCGGGAAGCCGCGCAGAAAG |
| | | AATCCCCAAGAGGGCCTGTACAACGAGCTCCAAAAGGATAAGATGGCA |
| | | GAAGCCTATAGCGAGATTGGTATGAAAGGGGAACGCAGAAGAGGCAAA |
| | | GGCCACGACGGACTGTACCAGGGACTCAGCACCGCCACCAAGGACACC |
| | | TATGACGCTCTTCACATGCAGGCCCTGCCGCCTCGG |

BCMA_EBB-C1978-A4

| | | |
|---|---|---|
| BCMA_EBB- | 432 | EVQLVESGGGLVQPGGSLRLSCAASGFTFSSYAMSWVRQAPGKGLEWV |
| C1978-A4- | | SAISGSGGSTYYADSVKGRFTISRDNSKNTLYLQMNSLRAEDTAVYYC |
| aa | | AKVEGSGSLDYWGQGTLVTVSSGGGGSGGGGSGGGGSEIVMTQSPGTL |
| ScFv | | SLSPGERATLSCRASQSVSSAYLAWYQQKPGQPPRLLISGASTRATGI |
| domain | | PDRFGGSGSGTDFTLTISRLEPEDFAVYYCQHYGSSFNGSSLFTFGQG |
| | | TRLEIK |
| BCMA_EBB- | 433 | GAAGTGCAGCTCGTGGAGTCAGGAGGCGGCCTGGTCCAGCCGGGAGGG |
| C1978-A4- | | TCCCTTAGACTGTCATGCGCCGCAAGCGGATTCACTTTCTCCTCCTAT |
| nt | | GCCATGAGCTGGGTCCGCCAAGCCCCCGGAAAGGGACTGGAATGGGTG |
| ScFv | | TCCGCCATCTCGGGGTCTGGAGGCTCAACTTACTACGCTGACTCCGTG |
| domain | | AAGGGACGGTTCACCATTAGCCGCGACAACTCCAAGAACACCCTCTAC |
| | | CTCCAAATGAACTCCCTGCGGGCCGAGGATACCGCCGTCTACTACTGC |
| | | GCCAAAGTGGAAGGTTCAGGATCGCTGGACTACTGGGGACAGGGTACT |
| | | CTCGTGACCGTGTCATCGGGCGGAGGAGGTTCCGGCGGTGGCGGCTCC |
| | | GGCGGCGGAGGGTCGGAGATCGTGATGACCCAGAGCCCTGGTACTCTG |
| | | AGCCTTTCGCCGGGAGAAAGGGCCACCCTGTCCTGCCGCGCTTCCCAA |
| | | TCCGTGTCCTCCGCGTACTTGGCGTGGTACCAGCAGAAGCCGGGACAG |
| | | CCCCCTCGGCTGCTGATCAGCGGGGCCAGCACCCGGGCAACCGGAATC |
| | | CCAGACAGATTCGGGGGTTCCGGCAGCGGCACAGATTTCACCCTGACT |
| | | ATTTCGAGGTTGGAGCCCGAGGACTTTGCGGTGTATTACTGTCAGCAC |
| | | TACGGGTCGTCCTTTAATGGCTCCAGCCTGTTCACGTTCGGACAGGGG |
| | | ACCCGCCTGGAAATCAAG |
| BCMA_EBB- | 434 | EVQLVESGGGLVQPGGSLRLSCAASGFTFSSYAMSWVRQAPGKGLEWV |
| C1978-A4- | | SAISGSGGSTYYADSVKGRFTISRDNSKNTLYLQMNSLRAEDTAVYYC |
| aa | | AKVEGSGSLDYWGQGTLVTVSS |
| VH | | |
| BCMA_EBB- | 435 | EIVMTQSPGTLSLSPGERATLSCRASQSVSSAYLAWYQQKPGQPPRLL |
| C1978-A4- | | ISGASTRATGIPDRFGGSGSGTDFTLTISRLEPEDFAVYYCQHYGSSF |
| aa | | NGSSLFTFGQGTRLEIK |
| VL | | |
| BCMA_EBB- | 436 | MALPVTALLLPLALLLHAARPEVQLVESGGGLVQPGGSLRLSCAASGF |
| C1978-A4- | | TFSSYAMSWVRQAPGKGLEWVSAISGSGGSTYYADSVKGRFTISRDNS |
| aa | | KNTLYLQMNSLRAEDTAVYYCAKVEGSGSLDYWGQGTLVTVSSGGGGS |
| Full CART | | GGGGSGGGGSEIVMTQSPGTLSLSPGERATLSCRASQSVSSAYLAWYQ |
| | | QKPGQPPRLLISGASTRATGIPDRFGGSGSGTDFTLTISRLEPEDFAV |
| | | YYCQHYGSSFNGSSLFTFGQGTRLEIKTTTPAPRPPTPAPTIASQPLS |
| | | LRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSR |
| | | SADAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDTYD |
| | | ALHMQALPPR |
| BCMA_EBB- | 437 | ATGGCCCTCCCTGTCACCGCCCTGCTGCTTCCGCTGGCTCTTCTGCTC |
| C1978-A4- | | CACGCCGCTCGGCCCGAAGTGCAGCTCGTGGAGTCAGGAGGCGGCCTG |
| nt | | GTCCAGCCGGGAGGGTCCCTTAGACTGTCATGCGCCGCAAGCGGATTC |
| Full CART | | ACTTTCTCCTCCTATGCCATGAGCTGGGTCCGCCAAGCCCCCGGAAAG |
| | | GGACTGGAATGGGTGTCCGCCATCTCGGGGTCTGGAGGCTCAACTTAC |
| | | TACGCTGACTCCGTGAAGGGACGGTTCACCATTAGCCGCGACAACTCC |
| | | AAGAACACCCTCTACCTCCAAATGAACTCCCTGCGGGCCGAGGATACC |
| | | GCCGTCTACTACTGCGCCAAAGTGGAAGGTTCAGGATCGCTGGACTAC |
| | | TGGGGACAGGGTACTCTCGTGACCGTGTCATCGGGCGGAGGAGGTTCC |
| | | GGCGGTGGCGGCTCCGGCGGCGGAGGGTCGGAGATCGTGATGACCCAG |
| | | AGCCCTGGTACTCTGAGCCTTTCGCCGGGAGAAAGGGCCACCCTGTCC |
| | | TGCCGCGCTTCCCAATCCGTGTCCTCCGCGTACTTGGCGTGGTACCAG |
| | | CAGAAGCCGGGACAGCCCCCTCGGCTGCTGATCAGCGGGGCCAGCACC |
| | | CGGGCAACCGGAATCCCAGACAGATTCGGGGGTTCCGGCAGCGGCACA |
| | | GATTTCACCCTGACTATTTCGAGGTTGGAGCCCGAGGACTTTGCGGTG |
| | | TATTACTGTCAGCACTACGGGTCGTCCTTTAATGGCTCCAGCCTGTTC |
| | | ACGTTCGGACAGGGGACCCGCCTGGAAATCAAGACCACTACCCCAGCA |
| | | CCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACC |
| | | CGGGGTCTTGACTTCGCCTGCGATATCTACATTTGGGCCCCTCTGGCT |
| | | GGTACTTGCGGGGTCCTGCTGCTTTCACTCGTGATCACTCTTTACTGT |
| | | AAGCGCGGTCGGAAGAAGCTGCTGTACATCTTTAAGCAACCCTTCATG |
| | | AGGCCTGTGCAGACTACTCAAGAGGAGGACGGCTGTTCATGCCGGTTC |
| | | CCAGAGGAGGAGGAAGGCGGCTGCGAACTGCGCGTGAAATTCAGCCGC |
| | | AGCGCAGATGCTCCAGCCTACAAGCAGGGGCAGAACCAGCTCTACAAC |
| | | GAACTCAATCTTGGTCGGAGAGAGGAGTACGACGTGCTGGACAAGCGG |
| | | AGAGGACGGGACCCAGAAATGGGCGGGAAGCCGCGCAGAAAGAATCCC |
| | | CAAGAGGGCCTGTACAACGAGCTCCAAAAGGATAAGATGGCAGAAGCC |
| | | TATAGCGAGATTGGTATGAAAGGGGAACGCAGAAGAGGCAAAGGCCAC |
| | | GACGGACTGTACCAGGGACTCAGCACCGCCACCAAGGACACCTATGAC |
| | | GCTCTTCACATGCAGGCCCTGCCGCCTCGG |

BCMA_EBB-C1978-G1

| | | |
|---|---|---|
| BCMA_EBB- | 438 | EVQLVETGGGLVQPGGSLRLSCAASGITFSRYPMSWVRQAPGKG |
| C1978-G1- | | LEWVSGISDSGVSTYYADSAKGRFTISRDNSKNTLFLQMSSLRDE |
| aa | | DTAVYYCVTRAGSEASDIWGQGTMVTVSSGGGGSGGGGSGGG |
| ScFv | | GSEIVLTQSPATLSLSPGERATLSCRASQSVSNSLAWYQQKPGQA |
| domain | | PRLLIYDASSRATGIPDRFSGSGSGTDFTLTISRLEPEDFAIYYCQQ |
| | | FGTSSGLTFGGGTKLEIK |
| BCMA_EBB- | 439 | GAAGTGCAACTGGTGGAAACCGGTGGCGGCCTGGTGCAGCCTGGAGGA |
| C1978-G1- | | TCATTGAGGCTGTCATGCGCGGCCAGCGGTATTACCTTCTCCCGGTAC |
| nt | | CCCATGTCCTGGGTCAGACAGGCCCCGGGGAAAGGGCTTGAATGGGTG |
| ScFv | | TCCGGGATCTCGGACTCCGGTGTCAGCACTTACTACGCCGACTCCGCC |
| domain | | AAGGGACGCTTCACCATTTCCCGGGACAACTCGAAGAACACCCTGTTC |
| | | CTCCAAATGAGCTCCCTCCGGGACGAGGATACTGCAGTGTACTACTGC |
| | | GTGACCCGCGCCGGGTCCGAGGCGTCTGACATTTGGGGACAGGGCACT |
| | | ATGGTCACCGTGTCGTCCGGCGGAGGGGGCTCGGGAGGCGGTGGCAGC |
| | | GGAGGAGGAGGGTCCGAGATCGTGCTGACCCAATCCCCGGCCACCCTC |
| | | TCGCTGAGCCCTGGAGAAAGGGCAACCTTGTCCTGTCGCGCGAGCCAG |
| | | TCCGTGAGCAACTCCCTGGCCTGGTACCAGCAGAAGCCCGGACAGGCT |
| | | CCGAGACTTCTGATCTACGACGCTTCGAGCCGGGCCACTGGAATCCCC |
| | | GACCGCTTTTCGGGGTCCGGCTCAGGAACCGATTTCACCCTGACAATC |
| | | TCACGGCTGGAGCCAGAGGATTTCGCCATCTATTACTGCCAGCAGTTC |
| | | GGTACTTCCTCCGGCCTGACTTTCGGAGGCGGCACGAAGCTCGAAATC |
| | | AAG |
| BCMA_EBB- | 440 | EVQLVETGGGLVQPGGSLRLSCAASGITFSRYPMSWVRQAPGKGLEWV |
| C1978-G1- | | SGISDSGVSTYYADSAKGRFTISRDNSKNTLFLQMSSLRDEDTAVYYC |
| aa | | VTRAGSEASDIWGQGTMVTVSS |
| VH | | |
| BCMA_EBB- | 441 | EIVLTQSPATLSLSPGERATLSCRASQSVSNSLAWYQQKPGQAPRLLI |
| C1978-G1- | | YDASSRATGIPDRFSGSGSGTDFTLTISRLEPEDFAIYYCQQFGTSSG |
| aa | | LTFGGGTKLEIK |
| VL | | |
| BCMA_EBB- | 442 | MALPVTALLLPLALLLHAARPEVQLVETGGGLVQPGGSLRLSCA |
| C1978-G1- | | ASGITFSRYPMSWVRQAPGKGLEWVSGISDSGVSTYYADSAKGR |
| aa | | FTISRDNSKNTLFLQMSSLRDEDTAVYYCVTRAGSEASDIWGQG |
| Full CART | | TMVTVSSGGGGSGGGGSGGGGSEIVLTQSPATLSLSPGERATLSC |
| | | RASQSVSNSLAWYQQKPGQAPRLLIYDASSRATGIPDRFSGSGSG |
| | | TDFTLTISRLEPEDFAIYYCQQFGTSSGLTFGGGTKLEIKTTTPAPR |
| | | PPTPAPTIASQPLSLRPEACRPAAGGAVHTRGLDFACDIYIWAPL |
| | | AGTCGVLLLSLVITLYCKRGRKKLLYIFKQPFMRPVQTTQEEDG |
| | | CSCRFPEEEEGGCELRVKFSRSADAPAYKQGQNQLYNELNLGRR |
| | | EEYDVLDKRRGRDPEMGGKPRRKNPQEGLYNELQKDKMAEAY |
| | | SEIGMKGERRRGKGHDGLYQGLSTATKDTYDALHMQALPPR |
| BCMA_EBB- | 443 | ATGGCCCTCCCTGTCACCGCCCTGCTGCTTCCGCTGGCTCTTCTGCTC |
| C1978-G1- | | CACGCCGCTCGGCCCGAAGTGCAACTGGTGGAAACCGGTGGCGGCCTG |
| nt | | GTGCAGCCTGGAGGATCATTGAGGCTGTCATGCGCGGCCAGCGGTATT |
| Full CART | | ACCTTCTCCCGGTACCCCATGTCCTGGGTCAGACAGGCCCCGGGGAAA |
| | | GGGCTTGAATGGGTGTCCGGGATCTCGGACTCCGGTGTCAGCACTTAC |
| | | TACGCCGACTCCGCCAAGGGACGCTTCACCATTTCCCGGGACAACTCG |
| | | AAGAACACCCTGTTCCTCCAAATGAGCTCCCTCCGGGACGAGGATACT |
| | | GCAGTGTACTACTGCGTGACCCGCGCCGGGTCCGAGGCGTCTGACATT |
| | | TGGGGACAGGGCACTATGGTCACCGTGTCGTCCGGCGGAGGGGGCTCG |
| | | GGAGGCGGTGGCAGCGGAGGAGGAGGGTCCGAGATCGTGCTGACCCAA |
| | | TCCCCGGCCACCCTCTCGCTGAGCCCTGGAGAAAGGGCAACCTTGTCC |
| | | TGTCGCGCGAGCCAGTCCGTGAGCAACTCCCTGGCCTGGTACCAGCAG |
| | | AAGCCCGGACAGGCTCCGAGACTTCTGATCTACGACGCTTCGAGCCGG |
| | | GCCACTGGAATCCCCGACCGCTTTTCGGGGTCCGGCTCAGGAACCGAT |
| | | TTCACCCTGACAATCTCACGGCTGGAGCCAGAGGATTTCGCCATCTAT |
| | | TACTGCCAGCAGTTCGGTACTTCCTCCGGCCTGACTTTCGGAGGCGGC |
| | | ACGAAGCTCGAAATCAAGACCACTACCCCAGCACCGAGGCCACCCACC |
| | | CCGGCTCCTACCATCGCCTCCCAGCCTCTGTCCCTGCGTCCGGAGGCA |
| | | TGTAGACCCGCAGCTGGTGGGGCCGTGCATACCCGGGGTCTTGACTTC |
| | | GCCTGCGATATCTACATTTGGGCCCCTCTGGCTGGTACTTGCGGGGTC |
| | | CTGCTGCTTTCACTCGTGATCACTCTTTACTGTAAGCGCGGTCGGAAG |
| | | AAGCTGCTGTACATCTTTAAGCAACCCTTCATGAGGCCTGTGCAGACT |
| | | ACTCAAGAGGAGGACGGCTGTTCATGCCGGTTCCCAGAGGAGGAGGAA |
| | | GGCGGCTGCGAACTGCGCGTGAAATTCAGCCGCAGCGCAGATGCTCCA |
| | | GCCTACAAGCAGGGGCAGAACCAGCTCTACAACGAACTCAATCTTGGT |
| | | CGGAGAGAGGAGTACGACGTGCTGGACAAGCGGAGAGGACGGGACCCA |
| | | GAAATGGGCGGGAAGCCGCGCAGAAAGAATCCCCAAGAGGGCCTGTAC |
| | | AACGAGCTCCAAAAGGATAAGATGGCAGAAGCCTATAGCGAGATTGGT |
| | | ATGAAAGGGGAACGCAGAAGAGGCAAAGGCCACGACGGACTGTACCAG |
| | | GGACTCAGCACCGCCACCAAGGACACCTATGACGCTCTTCACATGCAG |
| | | GCCCTGCCGCCTCGG |

BCMA_EBB-C1979-C1

| | | |
|---|---|---|
| BCMA_EBB- | 444 | QVQLVESGGGLVQPGGSLRLSCAASGFTFSSYAMSWVRQAPGKGLEWV |
| C1979-C1- | | SAISGSGGSTYYADSVKGRFTISRDNAKNSLYLQMNSLRAEDTAIYYC |
| aa | | ARATYKRELRYYYGMDVWGQGTMVTVSSGGGGSGGGGSGGGGSEIVMT |
| ScFv | | QSPGTVSLSPGERATLSCRASQSVSSSFLAWYQQKPGQAPRLLIYGAS |
| domain | | SRATGIPDRFSGSGSGTDFTLTISRLEPEDSAVYYCQQYHSSPSWTFG |
| | | QGTRLEIK |
| BCMA_EBB- | 445 | CAAGTGCAGCTCGTGGAATCGGGTGGCGGACTGGTGCAGCCGGGGGGC |
| C1979-C1- | | TCACTTAGACTGTCCTGCGCGGCCAGCGGATTCACTTTCTCCTCCTAC |
| nt | | GCCATGTCCTGGGTCAGACAGGCCCCTGGAAAGGGCCTGGAATGGGTG |
| ScFv | | TCCGCAATCAGCGGCAGCGGCGGCTCGACCTATTACGCGGATTCAGTG |
| domain | | AAGGGCAGATTCACCATTTCCCGGGACAACGCCAAGAACTCCTTGTAC |
| | | CTTCAAATGAACTCCCTCCGCGCGGAAGATACCGCAATCTACTACTGC |
| | | GCTCGGGCCACTTACAAGAGGGAACTGCGCTACTACTACGGGATGGAC |
| | | GTCTGGGGCCAGGGAACCATGGTCACCGTGTCCAGCGGAGGAGGAGGA |
| | | TCGGGAGGAGGCGGTAGCGGGGGTGGAGGGTCGGAGATCGTGATGACC |
| | | CAGTCCCCCGGCACTGTGTCGCTGTCCCCCGGCGAACGGGCCACCCTG |
| | | TCATGTCGGGCCAGCCAGTCAGTGTCGTCAAGCTTCCTCGCCTGGTAC |
| | | CAGCAGAAACCGGGACAAGCTCCCCGCCTGCTGATCTACGGAGCCAGC |
| | | AGCCGGGCCACCGGTATTCCTGACCGGTTCTCCGGTTCGGGGTCCGGG |
| | | ACCGACTTTACTCTGACTATCTCTCGCCTCGAGCCAGAGGACTCCGCC |
| | | GTGTATTACTGCCAGCAGTACCACTCCTCCCCGTCCTGGACGTTCGGA |
| | | CAGGGCACAAGGCTGGAGATTAAG |
| BCMA_EBB- | 446 | QVQLVESGGGLVQPGGSLRLSCAASGFTFSSYAMSWVRQAPGKGLEWV |
| C1979-C1- | | SAISGSGGSTYYADSVKGRFTISRDNAKNSLYLQMNSLRAEDTAIYYC |
| aa | | ARATYKRELRYYYGMDVWGQGTMVTVSS |
| VH | | |
| BCMA_EBB- | 447 | EIVMTQSPGTVSLSPGERATLSCRASQSVSSSFLAWYQQKPGQAPRLL |
| C1979-C1- | | IYGASSRATGIPDRFSGSGSGTDFTLTISRLEPEDSAVYYCQQYHSSP |
| aa | | SWTFGQGTRLEIK |
| VL | | |
| BCMA_EBB- | 448 | MALPVTALLLPLALLLHAARPQVQLVESGGGLVQPGGSLRLSCAASGF |
| C1979-C1- | | TFSSYAMSWVRQAPGKGLEWVSAISGSGGSTYYADSVKGRFTISRDNA |
| aa | | KNSLYLQMNSLRAEDTAIYYCARATYKRELRYYYGMDVWGQGTMVTVS |
| Full CART | | SGGGGSGGGGSGGGGSEIVMTQSPGTVSLSPGERATLSCRASQSVSSS |
| | | FLAWYQQKPGQAPRLLIYGASSRATGIPDRFSGSGSGTDFTLTISRLE |
| | | PEDSAVYYCQQYHSSPSWTFGQGTRLEIKTTTPAPRPPTPAPTIASQP |
| | | LSLRPEACRPAAGGAVHTRGLDFACDIYIWAPLAGTCGVLLLSLVITL |
| | | YCKRGRKKLLYIFKQPFMRPVQTTQEEDGCSCRFPEEEEGGCELRVKF |
| | | SRSADAPAYKQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPR |
| BCMA_EBB- | 449 | ATGGCCCTCCCTGTCACCGCCCTGCTGCTTCCGCTGGCTCTTCTGCTC |
| C1979-C1- | | CACGCCGCTCGGCCCCAAGTGCAGCTCGTGGAATCGGGTGGCGGACTG |
| nt | | GTGCAGCCGGGGGGCTCACTTAGACTGTCCTGCGCGGCCAGCGGATTC |
| Full CART | | ACTTTCTCCTCCTACGCCATGTCCTGGGTCAGACAGGCCCCTGGAAAG |
| | | GGCCTGGAATGGGTGTCCGCAATCAGCGGCAGCGGCGGCTCGACCTAT |
| | | TACGCGGATTCAGTGAAGGGCAGATTCACCATTTCCCGGGACAACGCC |
| | | AAGAACTCCTTGTACCTTCAAATGAACTCCCTCCGCGCGGAAGATACC |
| | | GCAATCTACTACTGCGCTCGGGCCACTTACAAGAGGGAACTGCGCTAC |
| | | TACTACGGGATGGACGTCTGGGGCCAGGGAACCATGGTCACCGTGTCC |
| | | AGCGGAGGAGGAGGATCGGGAGGAGGCGGTAGCGGGGGTGGAGGGTCG |
| | | GAGATCGTGATGACCCAGTCCCCCGGCACTGTGTCGCTGTCCCCCGGC |
| | | GAACGGGCCACCCTGTCATGTCGGGCCAGCCAGTCAGTGTCGTCAAGC |
| | | TTCCTCGCCTGGTACCAGCAGAAACCGGGACAAGCTCCCCGCCTGCTG |
| | | ATCTACGGAGCCAGCAGCCGGGCCACCGGTATTCCTGACCGGTTCTCC |
| | | GGTTCGGGGTCCGGGACCGACTTTACTCTGACTATCTCTCGCCTCGAG |
| | | CCAGAGGACTCCGCCGTGTATTACTGCCAGCAGTACCACTCCTCCCCG |
| | | TCCTGGACGTTCGGACAGGGCACAAGGCTGGAGATTAAGACCACTACC |
| | | CCAGCACCGAGGCCACCCACCCCGGCTCCTACCATCGCCTCCCAGCCT |
| | | CTGTCCCTGCGTCCGGAGGCATGTAGACCCGCAGCTGGTGGGGCCGTG |
| | | CATACCCGGGGTCTTGACTTCGCCTGCGATATCTACATTTGGGCCCCT |
| | | CTGGCTGGTACTTGCGGGGTCCTGCTGCTTTCACTCGTGATCACTCTT |
| | | TACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGC |
| | | CGGTTCCCAGAGGAGGAGGAAGGCGGCTGCGAACTGCGCGTGAAATTC |
| | | AGCCGCAGCGCAGATGCTCCAGCCTACAAGCAGGGGCAGAACCAGCTC |
| | | TACAACGAACTCAATCTTGGTCGGAGAGAGGAGTACGACGTGCTGGAC |
| | | AAGCGGAGAGGACGGGACCCAGAAATGGGCGGGAAGCCGCGCAGAAAG |
| | | AATCCCCAAGAGGGCCTGTACAACGAGCTCCAAAAGGATAAGATGGCA |
| | | GAAGCCTATAGCGAGATTGGTATGAAAGGGGAACGCAGAAGAGGCAAA |
| | | GGCCACGACGGACTGTACCAGGGACTCAGCACCGCCACCAAGGACACC |
| | | TATGACGCTCTTCACATGCAGGCCCTGCCGCCTCGG |

BCMA_EBB-C1978-C7

| | | |
|---|---|---|
| BCMA_EBB- | 450 | EVQLVETGGGLVQPGGSLRLSCAASGFTFSSYAMSWVRQAPGKGLEWV |
| C1978-C7- | | SAISGSGGSTYYADSVKGRFTISRDNSKNTLYLQMNTLKAEDTAVYYC |
| aa | | ARATYKRELRYYYGMDVWGQGTTVTVSSGGGGSGGGGSGGGGSEIVLT |
| ScFv | | QSPSTLSLSPGESATLSCRASQSVSTTFLAWYQQKPGQAPRLLIYGSS |
| domain | | NRATGIPDRFSGSGSGTDFTLTIRRLEPEDFAVYYCQQYHSSPSWTFG |
| | | QGTKVEIK |
| BCMA_EBB- | 451 | GAGGTGCAGCTTGTGGAAACCGGTGGCGGACTGGTGCAGCCCGGAGGA |
| C1978-C7- | | AGCCTCAGGCTGTCCTGCGCCGCGTCCGGCTTCACCTTCTCCTCGTAC |
| nt | | GCCATGTCCTGGGTCCGCCAGGCCCCCGGAAAGGGCCTGGAATGGGTG |
| ScFv | | TCCGCCATCTCTGGAAGCGGAGGTTCCACGTACTACGCGGACAGCGTC |
| domain | | AAGGGAAGGTTCACAATCTCCCGCGATAATTCGAAGAACACTCTGTAC |
| | | CTTCAAATGAACACCCTGAAGGCCGAGGACACTGCTGTGTACTACTGC |
| | | GCACGGGCCACCTACAAGAGAGAGCTCCGGTACTACTACGGAATGGAC |
| | | GTCTGGGGCCAGGGAACTACTGTGACCGTGTCCTCGGGAGGGGGTGGC |
| | | TCCGGGGGGGGCGGCTCCGGCGGAGGCGGTTCCGAGATTGTGCTGACC |
| | | CAGTCACCTTCAACTCTGTCGCTGTCCCCGGGAGAGAGCGCTACTCTG |
| | | AGCTGCCGGGCCAGCCAGTCCGTGTCCACCACCTTCCTCGCCTGGTAT |
| | | CAGCAGAAGCCGGGGCAGGCACCACGGCTCTTGATCTACGGGTCAAGC |
| | | AACAGAGCGACCGGAATTCCTGACCGCTTCTCGGGGAGCGGTTCAGGC |
| | | ACCGACTTCACCCTGACTATCCGGCGCCTGGAACCCGAAGATTTCGCC |
| | | GTGTATTACTGTCAACAGTACCACTCCTCGCCGTCCTGGACCTTTGGC |
| | | CAAGGAACCAAAGTGGAAATCAAG |
| BCMA_EBB- | 452 | EVQLVETGGGLVQPGGSLRLSCAASGFTFSSYAMSWVRQAPGKGLEWV |
| C1978-C7- | | SAISGSGGSTYYADSVKGRFTISRDNSKNTLYLQMNTLKAEDTAVYYC |
| aa | | ARATYKRELRYYYGMDVWGQGTTVTVSS |
| VH | | |
| BCMA_EBB- | 453 | EIVLTQSPSTLSLSPGESATLSCRASQSVSTTFLAWYQQKPGQAPRLL |
| C1978-C7- | | IYGSSNRATGIPDRFSGSGSGTDFTLTIRRLEPEDFAVYYCQQYHSSP |
| aa | | SWTFGQGTKVEIK |
| VL | | |
| BCMA_EBB- | 454 | MALPVTALLLPLALLLHAARPEVQLVETGGGLVQPGGSLRLSCAASGF |
| C1978-C7- | | TFSSYAMSWVRQAPGKGLEWVSAISGSGGSTYYADSVKGRFTISRDNS |
| aa | | KNTLYLQMNTLKAEDTAVYYCARATYKRELRYYYGMDVWGQGTTVTVS |
| Full CART | | SGGGGSGGGGSGGGGSEIVLTQSPSTLSLSPGESATLSCRASQSVSTT |
| | | FLAWYQQKPGQAPRLLIYGSSNRATGIPDRFSGSGSGTDFTLTIRRLE |
| | | PEDFAVYYCQQYHSSPSWTFGQGTKVEIKTTTPAPRPPTPAPTIASQP |
| | | LSLRPEACRPAAGGAVHTRGLDFACDIYIWAPLAGTCGVLLLSLVITL |
| | | YCKRGRKKLLYIFKQPFMRPVQTTQEEDGCSCRFPEEEEGGCELRVKF |
| | | SRSADAPAYKQGQNQLYNELNLGRREEYDVLDKRRGRDPEMGGKPRRK |
| | | NPQEGLYNELQKDKMAEAYSEIGMKGERRRGKGHDGLYQGLSTATKDT |
| | | YDALHMQALPPR |
| BCMA_EBB- | 455 | ATGGCCCTCCCTGTCACCGCCCTGCTGCTTCCGCTGGCTCTTCTGCTC |
| C1978-C7- | | CACGCCGCTCGGCCCGAGGTGCAGCTTGTGGAAACCGGTGGCGGACTG |
| nt | | GTGCAGCCCGGAGGAAGCCTCAGGCTGTCCTGCGCCGCGTCCGGCTTC |
| Full CART | | ACCTTCTCCTCGTACGCCATGTCCTGGGTCCGCCAGGCCCCCGGAAAG |
| | | GGCCTGGAATGGGTGTCCGCCATCTCTGGAAGCGGAGGTTCCACGTAC |
| | | TACGCGGACAGCGTCAAGGGAAGGTTCACAATCTCCCGCGATAATTCG |
| | | AAGAACACTCTGTACCTTCAAATGAACACCCTGAAGGCCGAGGACACT |
| | | GCTGTGTACTACTGCGCACGGGCCACCTACAAGAGAGAGCTCCGGTAC |
| | | TACTACGGAATGGACGTCTGGGGCCAGGGAACTACTGTGACCGTGTCC |
| | | TCGGGAGGGGGTGGCTCCGGGGGGGGCGGCTCCGGCGGAGGCGGTTCC |
| | | GAGATTGTGCTGACCCAGTCACCTTCAACTCTGTCGCTGTCCCCGGGA |
| | | GAGAGCGCTACTCTGAGCTGCCGGGCCAGCCAGTCCGTGTCCACCACC |
| | | TTCCTCGCCTGGTATCAGCAGAAGCCGGGGCAGGCACCACGGCTCTTG |
| | | ATCTACGGGTCAAGCAACAGAGCGACCGGAATTCCTGACCGCTTCTCG |
| | | GGGAGCGGTTCAGGCACCGACTTCACCCTGACTATCCGGCGCCTGGAA |
| | | CCCGAAGATTTCGCCGTGTATTACTGTCAACAGTACCACTCCTCGCCG |
| | | TCCTGGACCTTTGGCCAAGGAACCAAAGTGGAAATCAAGACCACTACC |
| | | CCAGCACCGAGGCCACCCACCCCGGCTCCTACCATCGCCTCCCAGCCT |
| | | CTGTCCCTGCGTCCGGAGGCATGTAGACCCGCAGCTGGTGGGGCCGTG |
| | | CATACCCGGGGTCTTGACTTCGCCTGCGATATCTACATTTGGGCCCCT |
| | | CTGGCTGGTACTTGCGGGGTCCTGCTGCTTTCACTCGTGATCACTCTT |
| | | TACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGC |
| | | CGGTTCCCAGAGGAGGAGGAAGGCGGCTGCGAACTGCGCGTGAAATTC |
| | | AGCCGCAGCGCAGATGCTCCAGCCTACAAGCAGGGGCAGAACCAGCTC |
| | | TACAACGAACTCAATCTTGGTCGGAGAGAGGAGTACGACGTGCTGGAC |
| | | AAGCGGAGAGGACGGGACCCAGAAATGGGCGGGAAGCCGCGCAGAAAG |
| | | AATCCCCAAGAGGGCCTGTACAACGAGCTCCAAAAGGATAAGATGGCA |
| | | GAAGCCTATAGCGAGATTGGTATGAAAGGGGAACGCAGAAGAGGCAAA |
| | | GGCCACGACGGACTGTACCAGGGACTCAGCACCGCCACCAAGGACACC |
| | | TATGACGCTCTTCACATGCAGGCCCTGCCGCCTCGG |

BCMA_EBB-C1978-D10

| | | |
|---|---|---|
| BCMA_EBB- | 456 | EVQLVETGGGLVQPGRSLRLSCAASGFTFDDYAMHWVRQAPGKGLEWV |
| C1978- | | SGISWNSGSIGYADSVKGRFTISRDNAKNSLYLQMNSLRDEDTAVYYC |
| D10 - aa | | ARVGKAVPDVWGQGTTVTVSSGGGGSGGGGSGGGGSDIVMTQTPSSLS |
| ScFv | | ASVGDRVTITCRASQSISSYLNWYQQKPGKAPKLLIYAASSLQSGVPS |
| domain | | RFSGSGSGTDFTLTISSLQPEDFATYYCQQSYSTPYSFGQGTRLEIK |
| BCMA_EBB- | 457 | GAAGTGCAGCTCGTGGAAACTGGAGGTGGACTCGTGCAGCCTGGACGG |
| C1978- | | TCGCTGCGGCTGAGCTGCGCTGCATCCGGCTTCACCTTCGACGATTAT |
| D10- nt | | GCCATGCACTGGGTCAGACAGGCGCCAGGGAAGGGACTTGAGTGGGTG |
| ScFv | | TCCGGTATCAGCTGGAATAGCGGCTCAATCGGATACGCGGACTCCGTG |
| domain | | AAGGGAAGGTTCACCATTTCCCGCGACAACGCCAAGAACTCCCTGTAC |
| | | TTGCAAATGAACAGCCTCCGGGATGAGGACACTGCCGTGTACTACTGC |
| | | GCCCGCGTCGGAAAAGCTGTGCCCGACGTCTGGGGCCAGGGAACCACT |
| | | GTGACCGTGTCCAGCGGCGGGGGTGGATCGGGCGGTGGAGGGTCCGGT |
| | | GGAGGGGGCTCAGATATTGTGATGACCCAGACCCCCTCGTCCCTGTCC |
| | | GCCTCGGTCGGCGACCGCGTGACTATCACATGTAGAGCCTCGCAGAGC |
| | | ATCTCCAGCTACCTGAACTGGTATCAGCAGAAGCCGGGGAAGGCCCCG |
| | | AAGCTCCTGATCTACGCGGCATCATCACTGCAATCGGGAGTGCCGAGC |
| | | CGGTTTTCCGGGTCCGGCTCCGGCACCGACTTCACGCTGACCATTTCT |
| | | TCCCTGCAACCCGAGGACTTCGCCACTTACTACTGCCAGCAGTCCTAC |
| | | TCCACCCCTTACTCCTTCGGCCAAGGAACCAGGCTGGAAATCAAG |
| BCMA_EBB- | 458 | EVQLVETGGGLVQPGRSLRLSCAASGFTFDDYAMHWVRQAPGKGLEWV |
| C1978- | | SGISWNSGSIGYADSVKGRFTISRDNAKNSLYLQMNSLRDEDTAVYYC |
| D10 - aa | | ARVGKAVPDVWGQGTTVTVSS |
| VH | | |
| BCMA_EBB- | 459 | DIVMTQTPSSLSASVGDRVTITCRASQSISSYLNWYQQKPGKAPKLLI |
| C1978- | | YAASSLQSGVPSRFSGSGSGTDFTLTISSLQPEDFATYYCQQSYSTPY |
| D10- aa | | SFGQGTRLEIK |
| VL | | |
| BCMA_EBB- | 460 | MALPVTALLLPLALLLHAARPEVQLVETGGGLVQPGRSLRLSCAASGF |
| C1978- | | TFDDYAMHWVRQAPGKGLEWVSGISWNSGSIGYADSVKGRFTISRDNA |
| D10 - aa | | KNSLYLQMNSLRDEDTAVYYCARVGKAVPDVWGQGTTVTVSSGGGGSG |
| Full CART | | GGGSGGGGSDIVMTQTPSSLSASVGDRVTITCRASQSISSYLNWYQQK |
| | | PGKAPKLLIYAASSLQSGVPSRFSGSGSGTDFTLTISSLQPEDFATYY |
| | | CQQSYSTPYSFGQGTRLEIKTTTPAPRPPTPAPTIASQPLSLRPEACR |
| | | PAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQAL |
| | | PPR |
| BCMA_EBB- | 461 | ATGGCCCTCCCTGTCACCGCCCTGCTGCTTCCGCTGGCTCTTCTGCTC |
| C1978- | | CACGCCGCTCGGCCCGAAGTGCAGCTCGTGGAAACTGGAGGTGGACTC |
| D10 - nt | | GTGCAGCCTGGACGGTCGCTGCGGCTGAGCTGCGCTGCATCCGGCTTC |
| Full CART | | ACCTTCGACGATTATGCCATGCACTGGGTCAGACAGGCGCCAGGGAAG |
| | | GGACTTGAGTGGGTGTCCGGTATCAGCTGGAATAGCGGCTCAATCGGA |
| | | TACGCGGACTCCGTGAAGGGAAGGTTCACCATTTCCCGCGACAACGCC |
| | | AAGAACTCCCTGTACTTGCAAATGAACAGCCTCCGGGATGAGGACACT |
| | | GCCGTGTACTACTGCGCCCGCGTCGGAAAAGCTGTGCCCGACGTCTGG |
| | | GGCCAGGGAACCACTGTGACCGTGTCCAGCGGCGGGGGTGGATCGGGC |
| | | GGTGGAGGGTCCGGTGGAGGGGGCTCAGATATTGTGATGACCCAGACC |
| | | CCCTCGTCCCTGTCCGCCTCGGTCGGCGACCGCGTGACTATCACATGT |
| | | AGAGCCTCGCAGAGCATCTCCAGCTACCTGAACTGGTATCAGCAGAAG |
| | | CCGGGGAAGGCCCCGAAGCTCCTGATCTACGCGGCATCATCACTGCAA |
| | | TCGGGAGTGCCGAGCCGGTTTTCCGGGTCCGGCTCCGGCACCGACTTC |
| | | ACGCTGACCATTTCTTCCCTGCAACCCGAGGACTTCGCCACTTACTAC |
| | | TGCCAGCAGTCCTACTCCACCCCTTACTCCTTCGGCCAAGGAACCAGG |
| | | CTGGAAATCAAGACCACTACCCCAGCACCGAGGCCACCCACCCCGGCT |
| | | CCTACCATCGCCTCCCAGCCTCTGTCCCTGCGTCCGGAGGCATGTAGA |
| | | CCCGCAGCTGGTGGGGCCGTGCATACCCGGGGTCTTGACTTCGCCTGC |
| | | GATATCTACATTTGGGCCCCTCTGGCTGGTACTTGCGGGGTCCTGCTG |
| | | CTTTCACTCGTGATCACTCTTTACTGTAAGCGCGGTCGGAAGAAGCTG |
| | | CTGTACATCTTTAAGCAACCCTTCATGAGGCCTGTGCAGACTACTCAA |
| | | GAGGAGGACGGCTGTTCATGCCGGTTCCCAGAGGAGGAGGAAGGCGGC |
| | | TGCGAACTGCGCGTGAAATTCAGCCGCAGCGCAGATGCTCCAGCCTAC |
| | | AAGCAGGGGCAGAACCAGCTCTACAACGAACTCAATCTTGGTCGGAGA |
| | | GAGGAGTACGACGTGCTGGACAAGCGGAGAGGACGGGACCCAGAAATG |
| | | GGCGGGAAGCCGCGCAGAAAGAATCCCCAAGAGGGCCTGTACAACGAG |
| | | CTCCAAAAGGATAAGATGGCAGAAGCCTATAGCGAGATTGGTATGAAA |
| | | GGGGAACGCAGAAGAGGCAAAGGCCACGACGGACTGTACCAGGGACTC |
| | | AGCACCGCCACCAAGGACACCTATGACGCTCTTCACATGCAGGCCCTG |
| | | CCGCCTCGG |

BCMA_EBB-C1979-C12

| | | |
|---|---|---|
| BCMA_EBB- | 462 | EVQLVESGGGLVQPGRSLRLSCTASGFTFDDYAMHWVRQRPGKGLEWV |
| C1979- | | ASINWKGNSLAYGDSVKGRFAISRDNAKNTVFLQMNSLRTEDTAVYYC |
| C12- aa | | ASHQGVAYYNYAMDVWGRGTLVTVSSGGGGSGGGGSGGGGSEIVLTQS |
| ScFv | | PGTLSLSPGERATLSCRATQSIGSSFLAWYQQRPGQAPRLLIYGASQR |
| domain | | ATGIPDRFSGRGSGTDFTLTISRVEPEDSAVYYCQHYESSPSWTFGQG |
| | | TKVEIK |
| BCMA_EBB- | 463 | GAAGTGCAGCTCGTGGAGAGCGGGGGAGGATTGGTGCAGCCCGGAAGG |
| C1979- | | TCCCTGCGGCTCTCCTGCACTGCGTCTGGCTTCACCTTCGACGACTAC |
| C12 - nt | | GCGATGCACTGGGTCAGACAGCGCCCGGGAAAGGGCCTGGAATGGGTC |
| ScFv | | GCCTCAATCAACTGGAAGGGAAACTCCCTGGCCTATGGCGACAGCGTG |
| domain | | AAGGGCCGCTTCGCCATTTCGCGCGACAACGCCAAGAACACCGTGTTT |
| | | CTGCAAATGAATTCCCTGCGGACCGAGGATACCGCTGTGTACTACTGC |
| | | GCCAGCCACCAGGGCGTGGCATACTATAACTACGCCATGGACGTGTGG |
| | | GGAAGAGGGACGCTCGTCACCGTGTCCTCCGGGGGCGGTGGATCGGGT |
| | | GGAGGAGGAAGCGGTGGCGGGGGCAGCGAAATCGTGCTGACTCAGAGC |
| | | CCGGGAACTCTTTCACTGTCCCCGGGAGAACGGGCCACTCTCTCGTGC |
| | | CGGGCCACCCAGTCCATCGGCTCCTCCTTCCTTGCCTGGTACCAGCAG |
| | | AGGCCAGGACAGGCGCCCCGCCTGCTGATCTACGGTGCTTCCCAACGC |
| | | GCCACTGGCATTCCTGACCGGTTCAGCGGCAGAGGGTCGGGAACCGAT |
| | | TTCACACTGACCATTTCCCGGGTGGAGCCCGAAGATTCGGCAGTCTAC |
| | | TACTGTCAGCATTACGAGTCCTCCCCTTCATGGACCTTCGGTCAAGGG |
| | | ACCAAAGTGGAGATCAAG |
| BCMA_EBB- | 464 | EVQLVESGGGLVQPGRSLRLSCTASGFTFDDYAMHWVRQRPGKGLEWV |
| C1979- | | ASINWKGNSLAYGDSVKGRFAISRDNAKNTVFLQMNSLRTEDTAVYYC |
| C12 - aa | | ASHQGVAYYNYAMDVWGRGTLVTVSS |
| VH | | |
| BCMA_EBB- | 465 | EIVLTQSPGTLSLSPGERATLSCRATQSIGSSFLAWYQQRPGQAPRLL |
| C1979- | | IYGASQRATGIPDRFSGRGSGTDFTLTISRVEPEDSAVYYCQHYESSP |
| C12 - aa | | SWTFGQGTKVEIK |
| VL | | |
| BCMA_EBB- | 466 | MALPVTALLLPLALLLHAARPEVQLVESGGGLVQPGRSLRLSCTASGF |
| C1979- | | TFDDYAMHWVRQRPGKGLEWVASINWKGNSLAYGDSVKGRFAISRDNA |
| C12 - aa | | KNTVFLQMNSLRTEDTAVYYCASHQGVAYYNYAMDVWGRGTLVTVSSG |
| Full CART | | GGGSGGGGSGGGGSEIVLTQSPGTLSLSPGERATLSCRATQSIGSSFL |
| | | AWYQQRPGQAPRLLIYGASQRATGIPDRFSGRGSGTDFTLTISRVEPE |
| | | DSAVYYCQHYESSPSWTFGQGTKVEIKTTTPAPRPPTPAPTIASQPLS |
| | | LRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSR |
| | | SADAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDTYD |
| | | ALHMQALPPR |
| BCMA_EBB- | 467 | ATGGCCCTCCCTGTCACCGCCCTGCTGCTTCCGCTGGCTCTTCTGCTC |
| C1979- | | CACGCCGCTCGGCCCGAAGTGCAGCTCGTGGAGAGCGGGGGAGGATTG |
| C12 - nt | | GTGCAGCCCGGAAGGTCCCTGCGGCTCTCCTGCACTGCGTCTGGCTTC |
| Full CART | | ACCTTCGACGACTACGCGATGCACTGGGTCAGACAGCGCCCGGGAAAG |
| | | GGCCTGGAATGGGTCGCCTCAATCAACTGGAAGGGAAACTCCCTGGCC |
| | | TATGGCGACAGCGTGAAGGGCCGCTTCGCCATTTCGCGCGACAACGCC |
| | | AAGAACACCGTGTTTCTGCAAATGAATTCCCTGCGGACCGAGGATACC |
| | | GCTGTGTACTACTGCGCCAGCCACCAGGGCGTGGCATACTATAACTAC |
| | | GCCATGGACGTGTGGGGAAGAGGGACGCTCGTCACCGTGTCCTCCGGG |
| | | GGCGGTGGATCGGGTGGAGGAGGAAGCGGTGGCGGGGGCAGCGAAATC |
| | | GTGCTGACTCAGAGCCCGGGAACTCTTTCACTGTCCCCGGGAGAACGG |
| | | GCCACTCTCTCGTGCCGGGCCACCCAGTCCATCGGCTCCTCCTTCCTT |
| | | GCCTGGTACCAGCAGAGGCCAGGACAGGCGCCCCGCCTGCTGATCTAC |
| | | GGTGCTTCCCAACGCGCCACTGGCATTCCTGACCGGTTCAGCGGCAGA |
| | | GGGTCGGGAACCGATTTCACACTGACCATTTCCCGGGTGGAGCCCGAA |
| | | GATTCGGCAGTCTACTACTGTCAGCATTACGAGTCCTCCCCTTCATGG |
| | | ACCTTCGGTCAAGGGACCAAAGTGGAGATCAAGACCACTACCCCAGCA |
| | | CCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACC |
| | | CGGGGTCTTGACTTCGCCTGCGATATCTACATTTGGGCCCCTCTGGCT |
| | | GGTACTTGCGGGGTCCTGCTGCTTTCACTCGTGATCACTCTTTACTGT |
| | | AAGCGCGGTCGGAAGAAGCTGCTGTACATCTTTAAGCAACCCTTCATG |
| | | AGGCCTGTGCAGACTACTCAAGAGGAGGACGGCTGTTCATGCCGGTTC |
| | | CCAGAGGAGGAGGAAGGCGGCTGCGAACTGCGCGTGAAATTCAGCCGC |
| | | AGCGCAGATGCTCCAGCCTACAAGCAGGGGCAGAACCAGCTCTACAAC |
| | | GAACTCAATCTTGGTCGGAGAGAGGAGTACGACGTGCTGGACAAGCGG |
| | | AGAGGACGGGACCCAGAAATGGGCGGGAAGCCGCGCAGAAAGAATCCC |
| | | CAAGAGGGCCTGTACAACGAGCTCCAAAAGGATAAGATGGCAGAAGCC |
| | | TATAGCGAGATTGGTATGAAAGGGGAACGCAGAAGAGGCAAAGGCCAC |
| | | GACGGACTGTACCAGGGACTCAGCACCGCCACCAAGGACACCTATGAC |
| | | GCTCTTCACATGCAGGCCCTGCCGCCTCGG |

BCMA_EBB-C1980-G4

| | | |
|---|---|---|
| BCMA_EBB- | 468 | EVQLVESGGGLVQPGGSLRLSCAASGFTFSSYAMSWVRQAPGK |
| C1980- | | GLEWVSAISGSGGSTYYADSVKGRFTISRDNSKNTLYLQMNSLR |
| G4- aa | | AEDTAVYYCAKVVRDGMDVWGQGTTVTVSSGGGGSGGGGSG |
| ScFv | | GGGSEIVLTQSPATLSLSPGERATLSCRASQSVSSSYLAWYQQKP |
| domain | | GQAPRLLIYGASSRATGIPDRFSGNGSGTDFTLTISRLEPEDFAVY |
| | | YCQQYGSPPRFTFGPGTKVDIK |
| BCMA_EBB- | 469 | GAGGTGCAGTTGGTCGAAAGCGGGGGCGGGCTTGTGCAGCCTGGCGGA |
| C1980- | | TCACTGCGGCTGTCCTGCGCGGCATCAGGCTTCACGTTTTCTTCCTAC |
| G4- nt | | GCCATGTCCTGGGTGCGCCAGGCCCCTGGAAAGGGACTGGAATGGGTG |
| ScFv | | TCCGCGATTTCGGGGTCCGGCGGGAGCACCTACTACGCCGATTCCGTG |
| domain | | AAGGGCCGCTTCACTATCTCGCGGGACAACTCCAAGAACACCCTCTAC |
| | | CTCCAAATGAATAGCCTGCGGGCCGAGGATACCGCCGTCTACTATTGC |
| | | GCTAAGGTCGTGCGCGACGGAATGGACGTGTGGGGACAGGGTACCACC |
| | | GTGACAGTGTCCTCGGGGGGAGGCGGTAGCGGCGGAGGAGGAAGCGGT |
| | | GGTGGAGGTTCCGAGATTGTGCTGACTCAATCACCCGCGACCCTGAGC |
| | | CTGTCCCCCGGCGAAAGGGCCACTCTGTCCTGTCGGGCCAGCCAATCA |
| | | GTCTCCTCCTCGTACCTGGCCTGGTACCAGCAGAAGCCAGGACAGGCT |
| | | CCGAGACTCCTTATCTATGGCGCATCCTCCCGCGCCACCGGAATCCCG |
| | | GATAGGTTCTCGGGAAACGGATCGGGGACCGACTTCACTCTCACCATC |
| | | TCCCGGCTGGAACCGGAGGACTTCGCCGTGTACTACTGCCAGCAGTAC |
| | | GGCAGCCCGCCTAGATTCACTTTCGGCCCCGGCACCAAAGTGGACATC |
| | | AAG |
| BCMA_EBB- | 470 | EVQLVESGGGLVQPGGSLRLSCAASGFTFSSYAMSWVRQAPGKGLEWV |
| C1980- | | SAISGSGGSTYYADSVKGRFTISRDNSKNTLYLQMNSLRAEDTAVYYC |
| G4- aa | | AKVVRDGMDVWGQGTTVTVSS |
| VH | | |
| BCMA_EBB- | 471 | EIVLTQSPATLSLSPGERATLSCRASQSVSSSYLAWYQQKPGQAPRLL |
| C1980- | | IYGASSRATGIPDRFSGNGSGTDFTLTISRLEPEDFAVYYCQQYGSPP |
| G4- aa | | RFTEGPGTKVDIK |
| VL | | |
| BCMA_EBB- | 472 | MALPVTALLLPLALLLHAARPEVQLVESGGGLVQPGGSLRLSCA |
| C1980- | | ASGFTFSSYAMSWVRQAPGKGLEWVSAISGSGGSTYYADSVKG |
| G4- aa | | RFTISRDNSKNTLYLQMNSLRAEDTAVYYCAKVVRDGMDVWG |
| Full CART | | QGTTVTVSSGGGGSGGGGSGGGGSEIVLTQSPATLSLSPGERATL |
| | | SCRASQSVSSSYLAWYQQKPGQAPRLLIYGASSRATGIPDRFSGN |
| | | GSGTDFTLTISRLEPEDFAVYYCQQYGSPPRFTFGPGTKVDIKTTT |
| | | PAPRPPTPAPTIASQPLSLRPEACRPAAGGAVHTRGLDFACDIYIW |
| | | APLAGTCGVLLLSLVITLYCKRGRKKLLYIFKQPFMRPVQTTQEE |
| | | DGCSCRFPEEEEGGCELRVKFSRSADAPAYKQGQNQLYNELNLG |
| | | RREEYDVLDKRRGRDPEMGGKPRRKNPQEGLYNELQKDKMAE |
| | | AYSEIGMKGERRRGKGHDGLYQGLSTATKDTYDALHMQALPPR |
| BCMA_EBB- | 473 | ATGGCCCTCCCTGTCACCGCCCTGCTGCTTCCGCTGGCTCTTCTGCTC |
| C1980- | | CACGCCGCTCGGCCCGAGGTGCAGTTGGTCGAAAGCGGGGGCGGGCTT |
| G4- nt | | GTGCAGCCTGGCGGATCACTGCGGCTGTCCTGCGCGGCATCAGGCTTC |
| Full CART | | ACGTTTTCTTCCTACGCCATGTCCTGGGTGCGCCAGGCCCCTGGAAAG |
| | | GGACTGGAATGGGTGTCCGCGATTTCGGGGTCCGGCGGGAGCACCTAC |
| | | TACGCCGATTCCGTGAAGGGCCGCTTCACTATCTCGCGGGACAACTCC |
| | | AAGAACACCCTCTACCTCCAAATGAATAGCCTGCGGGCCGAGGATACC |
| | | GCCGTCTACTATTGCGCTAAGGTCGTGCGCGACGGAATGGACGTGTGG |
| | | GGACAGGGTACCACCGTGACAGTGTCCTCGGGGGGAGGCGGTAGCGGC |
| | | GGAGGAGGAAGCGGTGGTGGAGGTTCCGAGATTGTGCTGACTCAATCA |
| | | CCCGCGACCCTGAGCCTGTCCCCCGGCGAAAGGGCCACTCTGTCCTGT |
| | | CGGGCCAGCCAATCAGTCTCCTCCTCGTACCTGGCCTGGTACCAGCAG |
| | | AAGCCAGGACAGGCTCCGAGACTCCTTATCTATGGCGCATCCTCCCGC |
| | | GCCACCGGAATCCCGGATAGGTTCTCGGGAAACGGATCGGGGACCGAC |
| | | TTCACTCTCACCATCTCCCGGCTGGAACCGGAGGACTTCGCCGTGTAC |
| | | TACTGCCAGCAGTACGGCAGCCCGCCTAGATTCACTTTCGGCCCCGGC |
| | | ACCAAAGTGGACATCAAGACCACTACCCCAGCACCGAGGCCACCCACC |
| | | CCGGCTCCTACCATCGCCTCCCAGCCTCTGTCCCTGCGTCCGGAGGCA |
| | | TGTAGACCCGCAGCTGGTGGGGCCGTGCATACCCGGGGTCTTGACTTC |
| | | GCCTGCGATATCTACATTTGGGCCCCTCTGGCTGGTACTTGCGGGGTC |
| | | CTGCTGCTTTCACTCGTGATCACTCTTTACTGTAAGCGCGGTCGGAAG |
| | | AAGCTGCTGTACATCTTTAAGCAACCCTTCATGAGGCCTGTGCAGACT |
| | | ACTCAAGAGGAGGACGGCTGTTCATGCCGGTTCCCAGAGGAGGAGGAA |
| | | GGCGGCTGCGAACTGCGCGTGAAATTCAGCCGCAGCGCAGATGCTCCA |
| | | GCCTACAAGCAGGGGCAGAACCAGCTCTACAACGAACTCAATCTTGGT |
| | | CGGAGAGAGGAGTACGACGTGCTGGACAAGCGGAGAGGACGGGACCCA |
| | | GAAATGGGCGGGAAGCCGCGCAGAAAGAATCCCCAAGAGGGCCTGTAC |
| | | AACGAGCTCCAAAAGGATAAGATGGCAGAAGCCTATAGCGAGATTGGT |
| | | ATGAAAGGGGAACGCAGAAGAGGCAAAGGCCACGACGGACTGTACCAG |
| | | GGACTCAGCACCGCCACCAAGGACACCTATGACGCTCTTCACATGCAG |
| | | GCCCTGCCGCCTCGG |

BCMA_EBB-C1980-D2

| | | |
|---|---|---|
| BCMA_EBB- | 474 | EVQLLESGGGLVQPGGSLRLSCAASGFTFSSYAMSWVRQAPGKGLEWV |
| C1980- | | SAISGSGGSTYYADSVKGRFTISRDNSKNTLYLQMNSLRAEDTAVYYC |
| D2- aa | | AKIPQTGTFDYWGQGTLVTVSSGGGGSGGGGSGGGGSEIVLTQSPGTL |
| ScFv | | SLSPGERATLSCRASQSVSSSYLAWYQQRPGQAPRLLIYGASSRATGI |
| domain | | PDRFSGSGSGTDFTLTISRLEPEDFAVYYCQHYGSSPSWTFGQGTRLE |
| | | IK |
| BCMA_EBB- | 475 | GAAGTGCAGCTGCTGGAGTCCGGCGGTGGATTGGTGCAACCGGGGGGA |
| C1980- | | TCGCTCAGACTGTCCTGTGCGGCGTCAGGCTTCACCTTCTCGAGCTAC |
| D2- nt | | GCCATGTCATGGGTCAGACAGGCCCCTGGAAAGGGTCTGGAATGGGTG |
| ScFv | | TCCGCCATTTCCGGGAGCGGGGGATCTACATACTACGCCGATAGCGTG |
| domain | | AAGGGCCGCTTCACCATTTCCCGGGACAACTCCAAGAACACTCTCTAT |
| | | CTGCAAATGAACTCCCTCCGCGCTGAGGACACTGCCGTGTACTACTGC |
| | | GCCAAAATCCCTCAGACCGGCACCTTCGACTACTGGGGACAGGGGACT |
| | | CTGGTCACCGTCAGCAGCGGTGGCGGAGGTTCGGGGGGAGGAGGAAGC |
| | | GGCGGCGGAGGGTCCGAGATTGTGCTGACCCAGTCACCCGGCACTTTG |
| | | TCCCTGTCGCCTGGAGAAAGGGCCACCCTTTCCTGCCGGGCATCCCAA |
| | | TCCGTGTCCTCCTCGTACCTGGCCTGGTACCAGCAGAGGCCCGGACAG |
| | | GCCCCACGGCTTCTGATCTACGGAGCAAGCAGCCGCGCGACCGGTATC |
| | | CCGGACCGGTTTTCGGGCTCGGGCTCAGGAACTGACTTCACCCTCACC |
| | | ATCTCCCGCCTGGAACCCGAAGATTTCGCTGTGTATTACTGCCAGCAC |
| | | TACGGCAGCTCCCCGTCCTGGACGTTCGGCCAGGGAACTCGGCTGGAG |
| | | ATCAAG |
| BCMA_EBB- | 476 | EVQLLESGGGLVQPGGSLRLSCAASGFTFSSYAMSWVRQAPGKGLEWV |
| C1980- | | SAISGSGGSTYYADSVKGRFTISRDNSKNTLYLQMNSLRAEDTAVYYC |
| D2- aa | | AKIPQTGTFDYWGQGTLVTVSS |
| VH | | |
| BCMA_EBB- | 477 | EIVLTQSPGTLSLSPGERATLSCRASQSVSSSYLAWYQQRPGQAPRLL |
| C1980- | | IYGASSRATGIPDRFSGSGSGTDFTLTISRLEPEDFAVYYCQHYGSSP |
| D2- aa | | SWTFGQGTRLEIK |
| VL | | |
| BCMA_EBB- | 478 | MALPVTALLLPLALLLHAARPEVQLLESGGGLVQPGGSLRLSCAASGF |
| C1980- | | TFSSYAMSWVRQAPGKGLEWVSAISGSGGSTYYADSVKGRFTISRDNS |
| D2- aa | | KNTLYLQMNSLRAEDTAVYYCAKIPQTGTFDYWGQGTLVTVSSGGGGS |
| Full CART | | GGGGSGGGGSEIVLTQSPGTLSLSPGERATLSCRASQSVSSSYLAWYQ |
| | | QRPGQAPRLLIYGASSRATGIPDRFSGSGSGTDFTLTISRLEPEDFAV |
| | | YYCQHYGSSPSWTFGQGTRLEIKTTTPAPRPPTPAPTIASQPLSLRPE |
| | | ACRPAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRSADA |
| | | PAYKQGQNQLYNELNLGRREEYDVLDKRRGRDPEMGGKPRRKNPQEGL |
| | | YNELQKDKMAEAYSEIGMKGERRRGKGHDGLYQGLSTATKDTYDALHM |
| | | QALPPR |
| BCMA_EBB- | 479 | ATGGCCCTCCCTGTCACCGCCCTGCTGCTTCCGCTGGCTCTTCTGCTC |
| C1980- | | CACGCCGCTCGGCCCGAAGTGCAGCTGCTGGAGTCCGGCGGTGGATTG |
| D2- nt | | GTGCAACCGGGGGGATCGCTCAGACTGTCCTGTGCGGCGTCAGGCTTC |
| Full CART | | ACCTTCTCGAGCTACGCCATGTCATGGGTCAGACAGGCCCCTGGAAAG |
| | | GGTCTGGAATGGGTGTCCGCCATTTCCGGGAGCGGGGGATCTACATAC |
| | | TACGCCGATAGCGTGAAGGGCCGCTTCACCATTTCCCGGGACAACTCC |
| | | AAGAACACTCTCTATCTGCAAATGAACTCCCTCCGCGCTGAGGACACT |
| | | GCCGTGTACTACTGCGCCAAAATCCCTCAGACCGGCACCTTCGACTAC |
| | | TGGGGACAGGGGACTCTGGTCACCGTCAGCAGCGGTGGCGGAGGTTCG |
| | | GGGGGAGGAGGAAGCGGCGGCGGAGGGTCCGAGATTGTGCTGACCCAG |
| | | TCACCCGGCACTTTGTCCCTGTCGCCTGGAGAAAGGGCCACCCTTTCC |
| | | TGCCGGGCATCCCAATCCGTGTCCTCCTCGTACCTGGCCTGGTACCAG |
| | | CAGAGGCCCGGACAGGCCCCACGGCTTCTGATCTACGGAGCAAGCAGC |
| | | CGCGCGACCGGTATCCCGGACCGGTTTTCGGGCTCGGGCTCAGGAACT |
| | | GACTTCACCCTCACCATCTCCCGCCTGGAACCCGAAGATTTCGCTGTG |
| | | TATTACTGCCAGCACTACGGCAGCTCCCCGTCCTGGACGTTCGGCCAG |
| | | GGAACTCGGCTGGAGATCAAGACCACTACCCCAGCACCGAGGCCACCC |
| | | ACCCCGGCTCCTACCATCGCCTCCCAGCCTCTGTCCCTGCGTCCGGAG |
| | | GCATGTAGACCCGCAGCTGGTGGGGCCGTGCATACCCGGGGTCTTGAC |
| | | TTCGCCTGCGATATCTACATTTGGGCCCCTCTGGCTGGTACTTGCGGG |
| | | GTCCTGCTGCTTTCACTCGTGATCACTCTTTACTGTAAGCGCGGTCGG |
| | | AAGAAGCTGCTGTACATCTTTAAGCAACCCTTCATGAGGCCTGTGCAG |
| | | ACTACTCAAGAGGAGGACGGCTGTTCATGCCGGTTCCCAGAGGAGGAG |
| | | GAAGGCGGCTGCGAACTGCGCGTGAAATTCAGCCGCAGCGCAGATGCT |
| | | CCAGCCTACAAGCAGGGGCAGAACCAGCTCTACAACGAACTCAATCTT |
| | | GGTCGGAGAGAGGAGTACGACGTGCTGGACAAGCGGAGAGGACGGGAC |
| | | CCAGAAATGGGCGGGAAGCCGCGCAGAAAGAATCCCCAAGAGGGCCTG |
| | | TACAACGAGCTCCAAAAGGATAAGATGGCAGAAGCCTATAGCGAGATT |
| | | GGTATGAAAGGGGAACGCAGAAGAGGCAAAGGCCACGACGGACTGTAC |
| | | CAGGGACTCAGCACCGCCACCAAGGACACCTATGACGCTCTTCACATG |
| | | CAGGCCCTGCCGCCTCGG |

BCMA_EBB-C1978-A10

| | | |
|---|---|---|
| BCMA_EBB- | 480 | EVQLVETGGGLVQPGGSLRLSCAASGFTFSSYAMSWVRQAPGK |
| C1978- | | GLEWVSAISGSGGSTYYADSVKGRFTMSRENDKNSVFLQMNSL |
| A10- aa | | RVEDTGVYYCARANYKRELRYYYGMDVWGQGTMVTVSSGGG |
| ScFv | | GSGGGGSGGGGSEIVMTQSPGTLSLSPGESATLSCRASQRVASN |
| domain | | YLAWYQHKPGQAPSLLISGASSRATGVPDRFSGSGSGTDFTLAIS |
| | | RLEPEDSAVYYCQHYDSSPSWTFGQGTKVEIK |
| BCMA_EBB- | 481 | GAAGTGCAACTGGTGGAAACCGGTGGAGGACTCGTGCAGCCTGGCGGC |
| C1978- | | AGCCTCCGGCTGAGCTGCGCCGCTTCGGGATTCACCTTTTCCTCCTAC |
| A10- nt | | GCGATGTCTTGGGTCAGACAGGCCCCCGGAAAGGGGCTGGAATGGGTG |
| ScFv | | TCAGCCATCTCCGGCTCCGGCGGATCAACGTACTACGCCGACTCCGTG |
| domain | | AAAGGCCGGTTCACCATGTCGCGCGAGAATGACAAGAACTCCGTGTTC |
| | | CTGCAAATGAACTCCCTGAGGGTGGAGGACACCGGAGTGTACTATTGT |
| | | GCGCGCGCCAACTACAAGAGAGAGCTGCGGTACTACTACGGAATGGAC |
| | | GTCTGGGGACAGGGAACTATGGTGACCGTGTCATCCGGTGGAGGGGGA |
| | | AGCGGCGGTGGAGGCAGCGGGGGCGGGGGTTCAGAAATTGTCATGACC |
| | | CAGTCCCCGGGAACTCTTTCCCTCTCCCCCGGGGAATCCGCGACTTTG |
| | | TCCTGCCGGGCCAGCCAGCGCGTGGCCTCGAACTACCTCGCATGGTAC |
| | | CAGCATAAGCCAGGCCAAGCCCCTTCCCTGCTGATTTCCGGGGCTAGC |
| | | AGCCGCGCCACTGGCGTGCCGGATAGGTTCTCGGGAAGCGGCTCGGGT |
| | | ACCGATTTCACCCTGGCAATCTCGCGGCTGGAACCGGAGGATTCGGCC |
| | | GTGTACTACTGCCAGCACTATGACTCATCCCCCTCCTGGACATTCGGA |
| | | CAGGGCACCAAGGTCGAGATCAAG |
| BCMA_EBB- | 482 | EVQLVETGGGLVQPGGSLRLSCAASGFTFSSYAMSWVRQAPGKGLEWV |
| C1978- | | SAISGSGGSTYYADSVKGRFTMSRENDKNSVFLQMNSLRVEDTGVYYC |
| A10- aa | | ARANYKRELRYYYGMDVWGQGTMVTVSS |
| VH | | |
| BCMA_EBB- | 483 | EIVMTQSPGTLSLSPGESATLSCRASQRVASNYLAWYQHKPGQAPSLL |
| C1978- | | ISGASSRATGVPDRFSGSGSGTDFTLAISRLEPEDSAVYYCQHYDSSP |
| A10- aa | | SWTFGQGTKVEIK |
| VL | | |
| BCMA_EBB- | 484 | MALPVTALLLPLALLLHAARPEVQLVETGGGLVQPGGSLRLSCA |
| C1978- | | ASGFTFSSYAMSWVRQAPGKGLEWVSAISGSGGSTYYADSVKG |
| A10- aa | | RFTMSRENDKNSVFLQMNSLRVEDTGVYYCARANYKRELRYY |
| Full CART | | YGMDVWGQGTMVTVSSGGGGSGGGGSGGGGSEIVMTQSPGTL |
| | | SLSPGESATLSCRASQRVASNYLAWYQHKPGQAPSLLISGASSRA |
| | | TGVPDRFSGSGSGTDFTLAISRLEPEDSAVYYCQHYDSSPSWTFG |
| | | QGTKVEIKTTTPAPRPPTPAPTIASQPLSLRPEACRPAAGGAVHTR |
| | | GLDFACDIYIWAPLAGTCGVLLLSLVITLYCKRGRKKLLYIFKQP |
| | | FMRPVQTTQEEDGCSCRFPEEEEGGCELRVKFSRSADAPAYKQG |
| | | QNQLYNELNLGRREEYDVLDKRRGRDPEMGGKPRRKNPQEGL |
| | | YNELQKDKMAEAYSEIGMKGERRRGKGHDGLYQGLSTATKDT |
| | | YDALHMQALPPR |
| BCMA_EBB- | 485 | ATGGCCCTCCCTGTCACCGCCCTGCTGCTTCCGCTGGCTCTTCTGCTC |
| C1978- | | CACGCCGCTCGGCCCGAAGTGCAACTGGTGGAAACCGGTGGAGGACTC |
| A10- nt | | GTGCAGCCTGGCGGCAGCCTCCGGCTGAGCTGCGCCGCTTCGGGATTC |
| Full CART | | ACCTTTTCCTCCTACGCGATGTCTTGGGTCAGACAGGCCCCCGGAAAG |
| | | GGGCTGGAATGGGTGTCAGCCATCTCCGGCTCCGGCGGATCAACGTAC |
| | | TACGCCGACTCCGTGAAAGGCCGGTTCACCATGTCGCGCGAGAATGAC |
| | | AAGAACTCCGTGTTCCTGCAAATGAACTCCCTGAGGGTGGAGGACACC |
| | | GGAGTGTACTATTGTGCGCGCGCCAACTACAAGAGAGAGCTGCGGTAC |
| | | TACTACGGAATGGACGTCTGGGGACAGGGAACTATGGTGACCGTGTCA |
| | | TCCGGTGGAGGGGGAAGCGGCGGTGGAGGCAGCGGGGGCGGGGGTTCA |
| | | GAAATTGTCATGACCCAGTCCCCGGGAACTCTTTCCCTCTCCCCCGGG |
| | | GAATCCGCGACTTTGTCCTGCCGGGCCAGCCAGCGCGTGGCCTCGAAC |
| | | TACCTCGCATGGTACCAGCATAAGCCAGGCCAAGCCCCTTCCCTGCTG |
| | | ATTTCCGGGGCTAGCAGCCGCGCCACTGGCGTGCCGGATAGGTTCTCG |
| | | GGAAGCGGCTCGGGTACCGATTTCACCCTGGCAATCTCGCGGCTGGAA |
| | | CCGGAGGATTCGGCCGTGTACTACTGCCAGCACTATGACTCATCCCCC |
| | | TCCTGGACATTCGGACAGGGCACCAAGGTCGAGATCAAGACCACTACC |
| | | CCAGCACCGAGGCCACCCACCCCGGCTCCTACCATCGCCTCCCAGCCT |
| | | CTGTCCCTGCGTCCGGAGGCATGTAGACCCGCAGCTGGTGGGGCCGTG |
| | | CATACCCGGGGTCTTGACTTCGCCTGCGATATCTACATTTGGGCCCCT |
| | | CTGGCTGGTACTTGCGGGGTCCTGCTGCTTTCACTCGTGATCACTCTT |
| | | TACTGTAAGCGCGGTCGGAAGAAGCTGCTGTACATCTTTAAGCAACCC |
| | | TTCATGAGGCCTGTGCAGACTACTCAAGAGGAGGACGGCTGTTCATGC |
| | | CGGTTCCCAGAGGAGGAGGAAGGCGGCTGCGAACTGCGCGTGAAATTC |
| | | AGCCGCAGCGCAGATGCTCCAGCCTACAAGCAGGGGCAGAACCAGCTC |
| | | TACAACGAACTCAATCTTGGTCGGAGAGAGGAGTACGACGTGCTGGAC |
| | | AAGCGGAGAGGACGGGACCCAGAAATGGGCGGGAAGCCGCGCAGAAAG |
| | | AATCCCCAAGAGGGCCTGTACAACGAGCTCCAAAAGGATAAGATGGCA |
| | | GAAGCCTATAGCGAGATTGGTATGAAAGGGGAACGCAGAAGAGGCAAA |
| | | GGCCACGACGGACTGTACCAGGGACTCAGCACCGCCACCAAGGACACC |
| | | TATGACGCTCTTCACATGCAGGCCCTGCCGCCTCGG |

BCMA_EBB-C1978-D4

| | | |
|---|---|---|
| BCMA_EBB- | 486 | EVQLLETGGGLVQPGGSLRLSCAASGFSFSSYAMSWVRQAPGKGLEWV |
| C1978- | | SAISGSGGSTYYADSVKGRFTISRDNSKNTLYLQMNSLRAEDTAVYYC |
| D4- aa | | AKALVGATGAFDIWGQGTLVTVSSGGGGSGGGGSGGGGSEIVLTQSPG |
| ScFv | | TLSLSPGERATLSCRASQSLSSNFLAWYQQKPGQAPGLLIYGASNWAT |
| domain | | GTPDRFSGSGSGTDFTLTITRLEPEDFAVYYCQYYGTSPMYTFGQGTK |
| | | VEIK |
| BCMA_EBB- | 487 | GAAGTGCAGCTGCTCGAAACCGGTGGAGGGCTGGTGCAGCCAGGGGGC |
| C1978- | | TCCCTGAGGCTTTCATGCGCCGCTAGCGGATTCTCCTTCTCCTCTTAC |
| D4- nt | | GCCATGTCGTGGGTCCGCCAAGCCCCTGGAAAAGGCCTGGAATGGGTG |
| ScFv | | TCCGCGATTTCCGGGAGCGGAGGTTCGACCTATTACGCCGACTCCGTG |
| domain | | AAGGGCCGCTTTACCATCTCCCGGGATAACTCCAAGAACACTCTGTAC |
| | | CTCCAAATGAACTCGCTGAGAGCCGAGGACACCGCCGTGTATTACTGC |
| | | GCGAAGGCGCTGGTCGGCGCGACTGGGGCATTCGACATCTGGGGACAG |
| | | GGAACTCTTGTGACCGTGTCGAGCGGAGGCGGCGGCTCCGGCGGAGGA |
| | | GGGAGCGGGGGCGGTGGTTCCGAAATCGTGTTGACTCAGTCCCCGGGA |
| | | ACCCTGAGCTTGTCACCCGGGGAGCGGGCCACTCTCTCCTGTCGCGCC |
| | | TCCCAATCGCTCTCATCCAATTTCCTGGCCTGGTACCAGCAGAAGCCC |
| | | GGACAGGCCCCGGGCCTGCTCATCTACGGCGCTTCAAACTGGGCAACG |
| | | GGAACCCCTGATCGGTTCAGCGGAAGCGGATCGGGTACTGACTTTACC |
| | | CTGACCATCACCAGACTGGAACCGGAGGACTTCGCCGTGTACTACTGC |
| | | CAGTACTACGGCACCTCCCCCATGTACACATTCGGACAGGGTACCAAG |
| | | GTCGAGATTAAG |
| BCMA_EBB- | 488 | EVQLLETGGGLVQPGGSLRLSCAASGFSFSSYAMSWVRQAPGKGLEWV |
| C1978- | | SAISGSGGSTYYADSVKGRFTISRDNSKNTLYLQMNSLRAEDTAVYYC |
| D4- aa | | AKALVGATGAFDIWGQGTLVTVSS |
| VH | | |
| BCMA_EBB- | 489 | EIVLTQSPGTLSLSPGERATLSCRASQSLSSNFLAWYQQKPGQAPGLL |
| C1978- | | IYGASNWATGTPDRFSGSGSGTDFTLTITRLEPEDFAVYYCQYYGTSP |
| D4- aa | | MYTFGQGTKVEIK |
| VL | | |
| BCMA_EBB- | 490 | MALPVTALLLPLALLLHAARPEVQLLETGGGLVQPGGSLRLSCAASGF |
| C1978- | | SFSSYAMSWVRQAPGKGLEWVSAISGSGGSTYYADSVKGRFTISRDNS |
| D4- aa | | KNTLYLQMNSLRAEDTAVYYCAKALVGATGAFDIWGQGTLVTVSSGGG |
| Full CART | | GSGGGGSGGGGSEIVLTQSPGTLSLSPGERATLSCRASQSLSSNFLAW |
| | | YQQKPGQAPGLLIYGASNWATGTPDRFSGSGSGTDFTLTITRLEPEDF |
| | | AVYYCQYYGTSPMYTFGQGTKVEIKTTTPAPRPPTPAPTIASQPLSLR |
| | | PEACRPAAGGAVHTRGLDFACDIYIWAPLAGTCGVLLLSLVITLYCKR |
| | | GRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYDAL |
| | | HMQALPPR |
| BCMA_EBB- | 491 | ATGGCCCTCCCTGTCACCGCCCTGCTGCTTCCGCTGGCTCTTCTGCTC |
| C1978- | | CACGCCGCTCGGCCCGAAGTGCAGCTGCTCGAAACCGGTGGAGGGCTG |
| D4- nt | | GTGCAGCCAGGGGGCTCCCTGAGGCTTTCATGCGCCGCTAGCGGATTC |
| Full CART | | TCCTTCTCCTCTTACGCCATGTCGTGGGTCCGCCAAGCCCCTGGAAAA |
| | | GGCCTGGAATGGGTGTCCGCGATTTCCGGGAGCGGAGGTTCGACCTAT |
| | | TACGCCGACTCCGTGAAGGGCCGCTTTACCATCTCCCGGGATAACTCC |
| | | AAGAACACTCTGTACCTCCAAATGAACTCGCTGAGAGCCGAGGACACC |
| | | GCCGTGTATTACTGCGCGAAGGCGCTGGTCGGCGCGACTGGGGCATTC |
| | | GACATCTGGGGACAGGGAACTCTTGTGACCGTGTCGAGCGGAGGCGGC |
| | | GGCTCCGGCGGAGGAGGGAGCGGGGGCGGTGGTTCCGAAATCGTGTTG |
| | | ACTCAGTCCCCGGGAACCCTGAGCTTGTCACCCGGGGAGCGGGCCACT |
| | | CTCTCCTGTCGCGCCTCCCAATCGCTCTCATCCAATTTCCTGGCCTGG |
| | | TACCAGCAGAAGCCCGGACAGGCCCCGGGCCTGCTCATCTACGGCGCT |
| | | TCAAACTGGGCAACGGGAACCCCTGATCGGTTCAGCGGAAGCGGATCG |
| | | GGTACTGACTTTACCCTGACCATCACCAGACTGGAACCGGAGGACTTC |
| | | GCCGTGTACTACTGCCAGTACTACGGCACCTCCCCCATGTACACATTC |
| | | GGACAGGGTACCAAGGTCGAGATTAAGACCACTACCCCAGCACCGAGG |
| | | CCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGT |
| | | CTTGACTTCGCCTGCGATATCTACATTTGGGCCCCTCTGGCTGGTACT |
| | | TGCGGGGTCCTGCTGCTTTCACTCGTGATCACTCTTTACTGTAAGCGC |
| | | GGTCGGAAGAAGCTGCTGTACATCTTTAAGCAACCCTTCATGAGGCCT |
| | | GTGCAGACTACTCAAGAGGAGGACGGCTGTTCATGCCGGTTCCCAGAG |
| | | GAGGAGGAAGGCGGCTGCGAACTGCGCGTGAAATTCAGCCGCAGCGCA |
| | | GATGCTCCAGCCTACAAGCAGGGGCAGAACCAGCTCTACAACGAACTC |
| | | AATCTTGGTCGGAGAGAGGAGTACGACGTGCTGGACAAGCGGAGAGGA |
| | | CGGGACCCAGAAATGGGCGGGAAGCCGCGCAGAAAGAATCCCCAAGAG |
| | | GGCCTGTACAACGAGCTCCAAAAGGATAAGATGGCAGAAGCCTATAGC |
| | | GAGATTGGTATGAAAGGGGAACGCAGAAGAGGCAAAGGCCACGACGGA |
| | | CTGTACCAGGGACTCAGCACCGCCACCAAGGACACCTATGACGCTCTT |
| | | CACATGCAGGCCCTGCCGCCTCGG |

BCMA_EBB-C1980-A2

| | | |
|---|---|---|
| BCMA_EBB- | 492 | EVQLLESGGGLVQPGGSLRLSCAASGFTFSSYAMSWVRQAPGKGLEWV |
| C1980- | | SAISGSGGSTYYADSVKGRFTISRDNSKNTLYLQMNSLRAEDTAVYYC |
| A2- aa | | VLWFGEGFDPWGQGTLVTVSSGGGGSGGGGSGGGGSDIVLTQSPLSLP |
| ScFv | | VTPGEPASISCRSSQSLLHSNGYNYLDWYLQKPGQSPQLLIYLGSNRA |
| domain | | SGVPDRFSGSGSGTDFTLKISRVEAEDVGVYYCMQALQTPLTFGGGTK |
| | | VDIK |
| BCMA_EBB- | 493 | GAAGTGCAGCTGCTTGAGAGCGGTGGAGGTCTGGTGCAGCCCGGGGGA |
| C1980- | | TCACTGCGCCTGTCCTGTGCCGCGTCCGGTTTCACTTTCTCCTCGTAC |
| A2- nt | | GCCATGTCGTGGGTCAGACAGGCACCGGGAAAGGGACTGGAATGGGTG |
| ScFv | | TCAGCCATTTCGGGTTCGGGGGGCAGCACCTACTACGCTGACTCCGTG |
| domain | | AAGGGCCGGTTCACCATTTCCCGCGACAACTCCAAGAACACCTTGTAC |
| | | CTCCAAATGAACTCCCTGCGGGCCGAAGATACCGCCGTGTATTACTGC |
| | | GTGCTGTGGTTCGGAGAGGGATTCGACCCGTGGGGACAAGGAACACTC |
| | | GTGACTGTGTCATCCGGCGGAGGCGGCAGCGGTGGCGGCGGTTCCGGC |
| | | GGCGGCGGATCTGACATCGTGTTGACCCAGTCCCCTCTGAGCCTGCCG |
| | | GTCACTCCTGGCGAACCAGCCAGCATCTCCTGCCGGTCGAGCCAGTCC |
| | | CTCCTGCACTCCAATGGGTACAACTACCTCGATTGGTATCTGCAAAAG |
| | | CCGGGCCAGAGCCCCCAGCTGCTGATCTACCTTGGGTCAAACCGCGCT |
| | | TCCGGGGTGCCTGATAGATTCTCCGGGTCCGGGAGCGGAACCGACTTT |
| | | ACCCTGAAAATCTCGAGGGTGGAGGCCGAGGACGTCGGAGTGTACTAC |
| | | TGCATGCAGGCGCTCCAGACTCCCCTGACCTTCGGAGGAGGAACGAAG |
| | | GTCGACATCAAGA |
| BCMA_EBB- | 494 | EVQLLESGGGLVQPGGSLRLSCAASGFTFSSYAMSWVRQAPGKGLEWV |
| C1980- | | SAISGSGGSTYYADSVKGRFTISRDNSKNTLYLQMNSLRAEDTAVYYC |
| A2- aa | | VLWFGEGFDPWGQGTLVTVSS |
| VH | | |
| BCMA_EBB- | 495 | DIVLTQSPLSLPVTPGEPASISCRSSQSLLHSNGYNYLDWYLQKPGQS |
| C1980- | | PQLLIYLGSNRASGVPDRFSGSGSGTDFTLKISRVEAEDVGVYYCMQA |
| A2- aa | | LQTPLTFGGGTKVDIK |
| VL | | |
| BCMA_EBB- | 496 | MALPVTALLLPLALLLHAARPEVQLLESGGGLVQPGGSLRLSCAASGF |
| C1980- | | TFSSYAMSWVRQAPGKGLEWVSAISGSGGSTYYADSVKGRFTISRDNS |
| A2- aa | | KNTLYLQMNSLRAEDTAVYYCVLWFGEGFDPWGQGTLVTVSSGGGGSG |
| Full CART | | GGGSGGGGSDIVLTQSPLSLPVTPGEPASISCRSSQSLLHSNGYNYLD |
| | | WYLQKPGQSPQLLIYLGSNRASGVPDRFSGSGSGTDFTLKISRVEAED |
| | | VGVYYCMQALQTPLTFGGGTKVDIKTTTPAPRPPTPAPTIASQPLSLR |
| | | PEACRPAAGGAVHTRGLDFACDIYIWAPLAGTCGVLLLSLVITLYCKR |
| | | GRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYDAL |
| | | HMQALPPR |
| BCMA_EBB- | 497 | ATGGCCCTCCCTGTCACCGCCCTGCTGCTTCCGCTGGCTCTTCTGCTC |
| C1980- | | CACGCCGCTCGGCCCGAAGTGCAGCTGCTTGAGAGCGGTGGAGGTCTG |
| A2- nt | | GTGCAGCCCGGGGGATCACTGCGCCTGTCCTGTGCCGCGTCCGGTTTC |
| Full CART | | ACTTTCTCCTCGTACGCCATGTCGTGGGTCAGACAGGCACCGGGAAAG |
| | | GGACTGGAATGGGTGTCAGCCATTTCGGGTTCGGGGGGCAGCACCTAC |
| | | TACGCTGACTCCGTGAAGGGCCGGTTCACCATTTCCCGCGACAACTCC |
| | | AAGAACACCTTGTACCTCCAAATGAACTCCCTGCGGGCCGAAGATACC |
| | | GCCGTGTATTACTGCGTGCTGTGGTTCGGAGAGGGATTCGACCCGTGG |
| | | GGACAAGGAACACTCGTGACTGTGTCATCCGGCGGAGGCGGCAGCGGT |
| | | GGCGGCGGTTCCGGCGGCGGCGGATCTGACATCGTGTTGACCCAGTCC |
| | | CCTCTGAGCCTGCCGGTCACTCCTGGCGAACCAGCCAGCATCTCCTGC |
| | | CGGTCGAGCCAGTCCCTCCTGCACTCCAATGGGTACAACTACCTCGAT |
| | | TGGTATCTGCAAAAGCCGGGCCAGAGCCCCCAGCTGCTGATCTACCTT |
| | | GGGTCAAACCGCGCTTCCGGGGTGCCTGATAGATTCTCCGGGTCCGGG |
| | | AGCGGAACCGACTTTACCCTGAAAATCTCGAGGGTGGAGGCCGAGGAC |
| | | GTCGGAGTGTACTACTGCATGCAGGCGCTCCAGACTCCCCTGACCTTC |
| | | GGAGGAGGAACGAAGGTCGACATCAAGACCACTACCCCAGCACCGAGG |
| | | CCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGGT |
| | | CTTGACTTCGCCTGCGATATCTACATTTGGGCCCCTCTGGCTGGTACT |
| | | TGCGGGGTCCTGCTGCTTTCACTCGTGATCACTCTTTACTGTAAGCGC |
| | | GGTCGGAAGAAGCTGCTGTACATCTTTAAGCAACCCTTCATGAGGCCT |
| | | GTGCAGACTACTCAAGAGGAGGACGGCTGTTCATGCCGGTTCCCAGAG |
| | | GAGGAGGAAGGCGGCTGCGAACTGCGCGTGAAATTCAGCCGCAGCGCA |
| | | GATGCTCCAGCCTACAAGCAGGGGCAGAACCAGCTCTACAACGAACTC |
| | | AATCTTGGTCGGAGAGAGGAGTACGACGTGCTGGACAAGCGGAGAGGA |
| | | CGGGACCCAGAAATGGGCGGGAAGCCGCGCAGAAAGAATCCCCAAGAG |
| | | GGCCTGTACAACGAGCTCCAAAAGGATAAGATGGCAGAAGCCTATAGC |
| | | GAGATTGGTATGAAAGGGGAACGCAGAAGAGGCAAAGGCCACGACGGA |
| | | CTGTACCAGGGACTCAGCACCGCCACCAAGGACACCTATGACGCTCTT |
| | | CACATGCAGGCCCTGCCGCCTCGG |

BCMA_EBB-C1981-C3

| | | |
|---|---|---|
| BCMA_EBB- | 498 | QVQLVESGGGLVQPGGSLRLSCAASGFTFSSYAMSWVRQAPGKGLEWV |
| C1981- | | SAISGSGGSTYYADSVKGRFTISRDNSKNTLYLQMNSLRAEDTAVYYC |
| C3- aa | | AKVGYDSSGYYRDYYGMDVWGQGTTVTVSSGGGGSGGGGSGGGGSEIV |
| ScFv | | LTQSPGTLSLSPGERATLSCRASQSVSSSYLAWYQQKPGQAPRLLIYG |
| domain | | TSSRATGISDRFSGSGSGTDFTLTISRLEPEDFAVYYCQHYGNSPPKF |
| | | TFGPGTKLEIK |
| BCMA_EBB- | 499 | CAAGTGCAGCTCGTGGAGTCAGGCGGAGGACTGGTGCAGCCCGGGGGC |
| C1981- | | TCCCTGAGACTTTCCTGCGCGGCATCGGGTTTTACCTTCTCCTCCTAT |
| C3- nt | | GCTATGTCCTGGGTGCGCCAGGCCCCGGGAAAGGGACTGGAATGGGTG |
| ScFv | | TCCGCAATCAGCGGTAGCGGGGGCTCAACATACTACGCCGACTCCGTC |
| domain | | AAGGGTCGCTTCACTATTTCCCGGGACAACTCCAAGAATACCCTGTAC |
| | | CTCCAAATGAACAGCCTCAGGGCCGAGGATACTGCCGTGTACTACTGC |
| | | GCCAAAGTCGGATACGATAGCTCCGGTTACTACCGGGACTACTACGGA |
| | | ATGGACGTGTGGGGACAGGGCACCACCGTGACCGTGTCAAGCGGCGGA |
| | | GGCGGTTCAGGAGGGGGAGGCTCCGGCGGTGGAGGGTCCGAAATCGTC |
| | | CTGACTCAGTCGCCTGGCACTCTGTCGTTGTCCCCGGGGGAGCGCGCT |
| | | ACCCTGTCGTGTCGGGCGTCGCAGTCCGTGTCGAGCTCCTACCTCGCG |
| | | TGGTACCAGCAGAAGCCCGGACAGGCCCCTAGACTTCTGATCTACGGC |
| | | ACTTCTTCACGCGCCACCGGGATCAGCGACAGGTTCAGCGGCTCCGGC |
| | | TCCGGGACCGACTTCACCCTGACCATTAGCCGGCTGGAGCCTGAAGAT |
| | | TTCGCCGTGTATTACTGCCAACACTACGGAAACTCGCCGCCAAAGTTC |
| | | ACGTTCGGACCCGGAACCAAGCTGGAAATCAAG |
| BCMA_EBB- | 500 | QVQLVESGGGLVQPGGSLRLSCAASGFTFSSYAMSWVRQAPGKGLEWV |
| C1981- | | SAISGSGGSTYYADSVKGRFTISRDNSKNTLYLQMNSLRAEDTAVYYC |
| C3- aa | | AKVGYDSSGYYRDYYGMDVWGQGTTVTVSS |
| VH | | |
| BCMA_EBB- | 501 | EIVLTQSPGTLSLSPGERATLSCRASQSVSSSYLAWYQQKPGQAPRLL |
| C1981- | | IYGTSSRATGISDRFSGSGSGTDFTLTISRLEPEDFAVYYCQHYGNSP |
| C3- aa | | PKFTFGPGTKLEIK |
| VL | | |
| BCMA_EBB- | 502 | MALPVTALLLPLALLLHAARPQVQLVESGGGLVQPGGSLRLSCAASGF |
| C1981- | | TFSSYAMSWVRQAPGKGLEWVSAISGSGGSTYYADSVKGRFTISRDNS |
| C3- aa | | KNTLYLQMNSLRAEDTAVYYCAKVGYDSSGYYRDYYGMDVWGQGTTVT |
| Full CART | | VSSGGGGSGGGGSGGGGSEIVLTQSPGTLSLSPGERATLSCRASQSVS |
| | | SSYLAWYQQKPGQAPRLLIYGTSSRATGISDRFSGSGSGTDFTLTISR |
| | | LEPEDFAVYYCQHYGNSPPKFTFGPGTKLEIKTTTPAPRPPTPAPTIA |
| | | SQPLSLRPEACRPAAGGAVHTRGLDFACDIYIWAPLAGTCGVLLLSLV |
| | | ITLYCKRGRKKLLYIFKQPFMRPVQTTQEEDGCSCRFPEEEEGGCELR |
| | | VKFSRSADAPAYKQGQNQLYNELNLGRREEYDVLDKRRGRDPEMGGKP |
| | | RRKNPQEGLYNELQKDKMAEAYSEIGMKGERRRGKGHDGLYQGLSTAT |
| | | KDTYDALHMQALPPR |
| BCMA_EBB- | 503 | ATGGCCCTCCCTGTCACCGCCCTGCTGCTTCCGCTGGCTCTTCTGCTC |
| C1981- | | CACGCCGCTCGGCCCCAAGTGCAGCTCGTGGAGTCAGGCGGAGGACTG |
| C3- nt | | GTGCAGCCCGGGGGCTCCCTGAGACTTTCCTGCGCGGCATCGGGTTTT |
| Full CART | | ACCTTCTCCTCCTATGCTATGTCCTGGGTGCGCCAGGCCCCGGGAAAG |
| | | GGACTGGAATGGGTGTCCGCAATCAGCGGTAGCGGGGGCTCAACATAC |
| | | TACGCCGACTCCGTCAAGGGTCGCTTCACTATTTCCCGGGACAACTCC |
| | | AAGAATACCCTGTACCTCCAAATGAACAGCCTCAGGGCCGAGGATACT |
| | | GCCGTGTACTACTGCGCCAAAGTCGGATACGATAGCTCCGGTTACTAC |
| | | CGGGACTACTACGGAATGGACGTGTGGGGACAGGGCACCACCGTGACC |
| | | GTGTCAAGCGGCGGAGGCGGTTCAGGAGGGGGAGGCTCCGGCGGTGGA |
| | | GGGTCCGAAATCGTCCTGACTCAGTCGCCTGGCACTCTGTCGTTGTCC |
| | | CCGGGGGAGCGCGCTACCCTGTCGTGTCGGGCGTCGCAGTCCGTGTCG |
| | | AGCTCCTACCTCGCGTGGTACCAGCAGAAGCCCGGACAGGCCCCTAGA |
| | | CTTCTGATCTACGGCACTTCTTCACGCGCCACCGGGATCAGCGACAGG |
| | | TTCAGCGGCTCCGGCTCCGGGACCGACTTCACCCTGACCATTAGCCGG |
| | | CTGGAGCCTGAAGATTTCGCCGTGTATTACTGCCAACACTACGGAAAC |
| | | TCGCCGCCAAAGTTCACGTTCGGACCCGGAACCAAGCTGGAAATCAAG |
| | | ACCACTACCCCAGCACCGAGGCCACCCACCCCGGCTCCTACCATCGCC |
| | | TCCCAGCCTCTGTCCCTGCGTCCGGAGGCATGTAGACCCGCAGCTGGT |
| | | GGGGCCGTGCATACCCGGGGTCTTGACTTCGCCTGCGATATCTACATT |
| | | TGGGCCCCTCTGGCTGGTACTTGCGGGGTCCTGCTGCTTTCACTCGTG |
| | | ATCACTCTTTACTGTAAGCGCGGTCGGAAGAAGCTGCTGTACATCTTT |
| | | AAGCAACCCTTCATGAGGCCTGTGCAGACTACTCAAGAGGAGGACGGC |
| | | TGTTCATGCCGGTTCCCAGAGGAGGAGGAAGGCGGCTGCGAACTGCGC |
| | | GTGAAATTCAGCCGCAGCGCAGATGCTCCAGCCTACAAGCAGGGGCAG |
| | | AACCAGCTCTACAACGAACTCAATCTTGGTCGGAGAGAGGAGTACGAC |
| | | GTGCTGGACAAGCGGAGAGGACGGGACCCAGAAATGGGCGGGAAGCCG |
| | | CGCAGAAAGAATCCCCAAGAGGGCCTGTACAACGAGCTCCAAAAGGAT |
| | | AAGATGGCAGAAGCCTATAGCGAGATTGGTATGAAAGGGGAACGCAGA |
| | | AGAGGCAAAGGCCACGACGGACTGTACCAGGGACTCAGCACCGCCACC |
| | | AAGGACACCTATGACGCTCTTCACATGCAGGCCCTGCCGCCTCGG |

BCMA_EBB-C1978-G4

| | | |
|---|---|---|
| BCMA_EBB- | 504 | EVQLVESGGGLVQPGGSLRLSCAASGFTFSSYAMSWVRQAPGKGLEWV |
| C1978- | | SAISGSGGSTYYADSVKGRFTISRDNSKNTLYLQMNSLRAEDTAVYYC |
| G4- aa | | AKMGWSSGYLGAFDIWGQGTTVTVSSGGGGSGGGGSGGGGSEIVLTQS |
| ScFv | | PGTLSLSPGERATLSCRASQSVASSFLAWYQQKPGQAPRLLIYGASGR |
| domain | | ATGIPDRFSGSGSGTDFTLTISRLEPEDFAVYYCQHYGGSPRLTFGGG |
| | | TKVDIK |
| BCMA_EBB- | 505 | GAAGTCCAACTGGTGGAGTCCGGGGGAGGGCTCGTGCAGCCCGGAGGC |
| C1978- | | AGCCTTCGGCTGTCGTGCGCCGCCTCCGGGTTCACGTTCTCATCCTAC |
| G4- nt | | GCGATGTCGTGGGTCAGACAGGCACCAGGAAAGGGACTGGAATGGGTG |
| ScFv | | TCCGCCATTAGCGGCTCCGGCGGTAGCACCTACTATGCCGACTCAGTG |
| domain | | AAGGGAAGGTTCACTATCTCCCGCGACAACAGCAAGAACACCCTGTAC |
| | | CTCCAAATGAACTCTCTGCGGGCCGAGGATACCGCGGTGTACTATTGC |
| | | GCCAAGATGGGTTGGTCCAGCGGATACTTGGGAGCCTTCGACATTTGG |
| | | GGACAGGGCACTACTGTGACCGTGTCCTCCGGGGGTGGCGGATCGGGA |
| | | GGCGGCGGCTCGGGTGGAGGGGGTTCCGAAATCGTGTTGACCCAGTCA |
| | | CCGGGAACCCTCTCGCTGTCCCCGGGAGAACGGGCTACACTGTCATGT |
| | | AGAGCGTCCCAGTCCGTGGCTTCCTCGTTCCTGGCCTGGTACCAGCAG |
| | | AAGCCGGGACAGGCACCCCGCCTGCTCATCTACGGAGCCAGCGGCCGG |
| | | GCGACCGGCATCCCTGACCGCTTCTCCGGTTCCGGCTCGGGCACCGAC |
| | | TTTACTCTGACCATTAGCAGGCTTGAGCCCGAGGATTTTGCCGTGTAC |
| | | TACTGCCAACACTACGGGGGGAGCCCTCGCCTGACCTTCGGAGGCGGA |
| | | ACTAAGGTCGATATCAAAA |
| BCMA_EBB- | 506 | EVQLVESGGGLVQPGGSLRLSCAASGFTFSSYAMSWVRQAPGKGLEWV |
| C1978- | | SAISGSGGSTYYADSVKGRFTISRDNSKNTLYLQMNSLRAEDTAVYYC |
| G4- aa | | AKMGWSSGYLGAFDIWGQGTTVTVSS |
| VH | | |
| BCMA_EBB- | 507 | EIVLTQSPGTLSLSPGERATLSCRASQSVASSFLAWYQQKPGQAPRLL |
| C1978- | | IYGASGRATGIPDRFSGSGSGTDFTLTISRLEPEDFAVYYCQHYGGSP |
| G4- aa | | RLTFGGGTKVDIK |
| VL | | |
| BCMA_EBB- | 508 | MALPVTALLLPLALLLHAARPEVQLVESGGGLVQPGGSLRLSCAASGF |
| C1978- | | TFSSYAMSWVRQAPGKGLEWVSAISGSGGSTYYADSVKGRFTISRDNS |
| G4- aa | | KNTLYLQMNSLRAEDTAVYYCAKMGWSSGYLGAFDIWGQGTTVTVSSG |
| Full CART | | GGGSGGGGSGGGGSEIVLTQSPGTLSLSPGERATLSCRASQSVASSFL |
| | | AWYQQKPGQAPRLLIYGASGRATGIPDRFSGSGSGTDFTLTISRLEPE |
| | | DFAVYYCQHYGGSPRLTFGGGTKVDIKTTTPAPRPPTPAPTIASQPLS |
| | | LRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSR |
| | | SADAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDTYD |
| | | ALHMQALPPR |
| BCMA_EBB- | 509 | ATGGCCCTCCCTGTCACCGCCCTGCTGCTTCCGCTGGCTCTTCTGCTC |
| C1978- | | CACGCCGCTCGGCCCGAAGTCCAACTGGTGGAGTCCGGGGGAGGGCTC |
| G4- nt | | GTGCAGCCCGGAGGCAGCCTTCGGCTGTCGTGCGCCGCCTCCGGGTTC |
| Full CART | | ACGTTCTCATCCTACGCGATGTCGTGGGTCAGACAGGCACCAGGAAAG |
| | | GGACTGGAATGGGTGTCCGCCATTAGCGGCTCCGGCGGTAGCACCTAC |
| | | TATGCCGACTCAGTGAAGGGAAGGTTCACTATCTCCCGCGACAACAGC |
| | | AAGAACACCCTGTACCTCCAAATGAACTCTCTGCGGGCCGAGGATACC |
| | | GCGGTGTACTATTGCGCCAAGATGGGTTGGTCCAGCGGATACTTGGGA |
| | | GCCTTCGACATTTGGGGACAGGGCACTACTGTGACCGTGTCCTCCGGG |
| | | GGTGGCGGATCGGGAGGCGGCGGCTCGGGTGGAGGGGGTTCCGAAATC |
| | | GTGTTGACCCAGTCACCGGGAACCCTCTCGCTGTCCCCGGGAGAACGG |
| | | GCTACACTGTCATGTAGAGCGTCCCAGTCCGTGGCTTCCTCGTTCCTG |
| | | GCCTGGTACCAGCAGAAGCCGGGACAGGCACCCCGCCTGCTCATCTAC |
| | | GGAGCCAGCGGCCGGGCGACCGGCATCCCTGACCGCTTCTCCGGTTCC |
| | | GGCTCGGGCACCGACTTTACTCTGACCATTAGCAGGCTTGAGCCCGAG |
| | | GATTTTGCCGTGTACTACTGCCAACACTACGGGGGGAGCCCTCGCCTG |
| | | ACCTTCGGAGGCGGAACTAAGGTCGATATCAAAACCACTACCCCAGCA |
| | | CCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACC |
| | | CGGGGTCTTGACTTCGCCTGCGATATCTACATTTGGGCCCCTCTGGCT |
| | | GGTACTTGCGGGGTCCTGCTGCTTTCACTCGTGATCACTCTTTACTGT |
| | | AAGCGCGGTCGGAAGAAGCTGCTGTACATCTTTAAGCAACCCTTCATG |
| | | AGGCCTGTGCAGACTACTCAAGAGGAGGACGGCTGTTCATGCCGGTTC |
| | | CCAGAGGAGGAGGAAGGCGGCTGCGAACTGCGCGTGAAATTCAGCCGC |
| | | AGCGCAGATGCTCCAGCCTACAAGCAGGGGCAGAACCAGCTCTACAAC |
| | | GAACTCAATCTTGGTCGGAGAGAGGAGTACGACGTGCTGGACAAGCGG |
| | | AGAGGACGGGACCCAGAAATGGGCGGGAAGCCGCGCAGAAAGAATCCC |
| | | CAAGAGGGCCTGTACAACGAGCTCCAAAAGGATAAGATGGCAGAAGCC |
| | | TATAGCGAGATTGGTATGAAAGGGGAACGCAGAAGAGGCAAAGGCCAC |
| | | GACGGACTGTACCAGGGACTCAGCACCGCCACCAAGGACACCTATGAC |
| | | GCTCTTCACATGCAGGCCCTGCCGCCTCGG |

TABLE 8
Additional exemplary BCMA CAR sequences

| | | SEQ |
|---|---|---|
| | | ID |
| Name | Sequence | NO: |
| A7D12.2 | QIQLVQSGPDLKKPGETVKLSCKASGYTFTNFGMNWVKQAPGKGFKWMAWINTYTGESYFA | 510 |
| VH | DDFKGRFAFSVETSATTAYLQINNLKTEDTATYFCARGEIYYGYDGGFAYWGQGTLVTVSA | |
| A7D12.2 | DVVMTQSHRFMSTSVGDRVSITCRASQDVNTAVSWYQQKPGQSPKLLIFSASYRYTGVPDR | 511 |
| VL | FTGSGSGADFTLTISSVQAEDLAVYYCQQHYSTPWTFGGGTKLDIK | |
| A7D12.2 | QIQLVQSGPDLKKPGETVKLSCKASGYTFTNFGMNWVKQAPGKGFKWMAWINTYTGESYFA | 512 |
| scFv | DDFKGRFAFSVETSATTAYLQINNLKTEDTATYFCARGEIYYGYDGGFAYWGQGTLVTVSA | |
| domain | GGGGSGGGGSGGGGSDVVMTQSHRFMSTSVGDRVSITCRASQDVNTAVSWYQQKPGQSPKL | |
| | LIFSASYRYTGVPDRFTGSGSGADFTLTISSVQAEDLAVYYCQQHYSTPWTFGGGTKLDIK | |
| A7D12.2 | QIQLVQSGPDLKKPGETVKLSCKASGYTFTNFGMNWVKQAPGKGFKWMAWINTYTGESYFA | 513 |
| Full | DDFKGRFAFSVETSATTAYLQINNLKTEDTATYFCARGEIYYGYDGGFAYWGQGTLVTVSA | |
| CART | GGGGSGGGGSGGGGSDVVMTQSHRFMSTSVGDRVSITCRASQDVNTAVSWYQQKPGQSPKL | |
| | LIFSASYRYTGVPDRFTGSGSGADFTLTISSVQAEDLAVYYCQQHYSTPWTFGGGTKLDIK | |
| | TTTPAPRPPTPAPTIASQPLSLRPEACRPAAGGAVHTRGLDFACDIYIWAPLAGTCGVLLL | |
| | SLVITLYCKRGRKKLLYIFKQPFMRPVQTTQEEDGCSCRFPEEEEGGCELRVKFSRSADAP | |
| | AYKQGQNQLYNELNLGRREEYDVLDKRRGRDPEMGGKPRRKNPQEGLYNELQKDKMAEAYS | |
| | EIGMKGERRRGKGHDGLYQGLSTATKDTYDALHMQALPPR | |
| C11D5.3 | QIQLVQSGPELKKPGETVKISCKASGYTFTDYSINWVKRAPGKGLKWMGWINTETREPAYA | 514 |
| VH | YDFRGRFAFSLETSASTAYLQINNLKYEDTATYFCALDYSYAMDYWGQGTSVTVSS | |
| C11D5.3 | DIVLTQSPASLAMSLGKRATISCRASESVSVIGAHLIHWYQQKPGQPPKLLIYLASNLETG | 515 |
| VL | VPARFSGSGSGTDFTLTIDPVEEDDVAIYSCLQSRIFPRTFGGGTKLEIK | |
| C11D5.3 | QIQLVQSGPELKKPGETVKISCKASGYTFTDYSINWVKRAPGKGLKWMGWINTETREPAYA | 516 |
| scFv | YDFRGRFAFSLETSASTAYLQINNLKYEDTATYFCALDYSYAMDYWGQGTSVTVSSGGGGS | |
| domain | GGGGSGGGGSQIQLVQSGPELKKPGETVKISCKASGYTFTDYSINWVKRAPGKGLKWMGWI | |
| | NTETREPAYAYDFRGRFAFSLETSASTAYLQINNLKYEDTATYFCALDYSYAMDYWGQGTS | |
| | VTVSS | |
| C11D5.3 | QIQLVQSGPELKKPGETVKISCKASGYTFTDYSINWVKRAPGKGLKWMGWINTETREPAYA | 517 |
| Full | YDFRGRFAFSLETSASTAYLQINNLKYEDTATYFCALDYSYAMDYWGQGTSVTVSSGGGGS | |
| CART | GGGGSGGGGSQIQLVQSGPELKKPGETVKISCKASGYTFTDYSINWVKRAPGKGLKWMGWI | |
| | NTETREPAYAYDFRGRFAFSLETSASTAYLQINNLKYEDTATYFCALDYSYAMDYWGQGTS | |
| | VTVSSTTTPAPRPPTPAPTIASQPLSLRPEACRPAAGGAVHTRGLDFACDIYIWAPLAGTC | |
| | GVLLLSLVITLYCKRGRKKLLYIFKQPFMRPVQTTQEEDGCSCRFPEEEEGGCELRVKFSR | |
| | SADAPAYKQGQNQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| C12A3.2 | QIQLVQSGPELKKPGETVKISCKASGYTFRHYSMNWVKQAPGKGLKWMGRINTESGVPIYA | 518 |
| VH | DDFKGRFAFSVETSASTAYLVINNLKDEDTASYFCSNDYLYSLDFWGQGTALTVSS | |
| C12A3.2 | DIVLTQSPPSLAMSLGKRATISCRASESVTILGSHLIYWYQQKPGQPPTLLIQLASNVQTG | 519 |
| VL | VPARFSGSGSRTDFTLTIDPVEEDDVAVYYCLQSRTIPRTFGGGTKLEIK | |
| C12A3.2 | QIQLVQSGPELKKPGETVKISCKASGYTFRHYSMNWVKQAPGKGLKWMGRINTESGVPIYA | 520 |
| scFv | DDFKGRFAFSVETSASTAYLVINNLKDEDTASYFCSNDYLYSLDFWGQGTALTVSSGGGGS | |
| domain | GGGGSGGGGSDIVLTQSPPSLAMSLGKRATISCRASESVTILGSHLIYWYQQKPGQPPTLL | |
| | IQLASNVQTGVPARFSGSGSRTDFTLTIDPVEEDDVAVYYCLQSRTIPRTFGGGTKLEIK | |
| C12A3.2 | QIQLVQSGPELKKPGETVKISCKASGYTFRHYSMNWVKQAPGKGLKWMGRINTESGVPIYA | 521 |
| Full | DDFKGRFAFSVETSASTAYLVINNLKDEDTASYFCSNDYLYSLDFWGQGTALTVSSGGGGS | |
| CART | GGGGSGGGGSDIVLTQSPPSLAMSLGKRATISCRASESVTILGSHLIYWYQQKPGQPPTLL | |
| | IQLASNVQTGVPARFSGSGSRTDFTLTIDPVEEDDVAVYYCLQSRTIPRTFGGGTKLEIKT | |
| | TTPAPRPPTPAPTIASQPLSLRPEACRPAAGGAVHTRGLDFACDIYIWAPLAGTCGVLLLS | |
| | LVITLYCKRGRKKLLYIFKQPFMRPVQTTQEEDGCSCRFPEEEEGGCELRVKFSRSADAPA | |
| | YKQGQNQLYNELNLGRREEYDVLDKRRGRDPEMGGKPRRKNPQEGLYNELQKDKMAEAYSE | |
| | IGMKGERRRGKGHDGLYQGLSTATKDTYDALHMQALPPR | |
| C13F12. | QIQLVQSGPELKKPGETVKISCKASGYTFTHYSMNWVKQAPGKGLKWMGRINTETGEPLYA | 522 |
| 1 VH | DDFKGRFAFSLETSASTAYLVINNLKNEDTATFFCSNDYLYSCDYWGQGTTLTVSS | |
| C13F12. | DIVLTQSPPSLAMSLGKRATISCRASESVTILGSHLIYWYQQKPGQPPTLLIQLASNVQTG | 523 |
| 1 VL | VPARFSGSGSRTDFTLTIDPVEEDDVAVYYCLQSRTIPRTFGGGTKLEIK | |
| C13F12.1 | QIQLVQSGPELKKPGETVKISCKASGYTFTHYSMNWVKQAPGKGLKWMGRINTETGEPLYA | 524 |
| scFv | DDFKGRFAFSLETSASTAYLVINNLKNEDTATFFCSNDYLYSCDYWGQGTTLTVSSGGGGS | |
| domain | GGGGSGGGGSDIVLTQSPPSLAMSLGKRATISCRASESVTILGSHLIYWYQQKPGQPPTLL | |
| | IQLASNVQTGVPARFSGSGSRTDFTLTIDPVEEDDVAVYYCLQSRTIPRTFGGGTKLEIK | |
| C13F12.1 | QIQLVQSGPELKKPGETVKISCKASGYTFTHYSMNWVKQAPGKGLKWMGRINTETGEPLYA | 525 |
| Full | DDFKGRFAFSLETSASTAYLVINNLKNEDTATFFCSNDYLYSCDYWGQGTTLTVSSGGGGS | |
| CART | GGGGSGGGGSDIVLTQSPPSLAMSLGKRATISCRASESVTILGSHLIYWYQQKPGQPPTLL | |
| | IQLASNVQTGVPARFSGSGSRTDFTLTIDPVEEDDVAVYYCLQSRTIPRTFGGGTKLEIKT | |
| | TTPAPRPPTPAPTIASQPLSLRPEACRPAAGGAVHTRGLDFACDIYIWAPLAGTCGVLLLS | |
| | LVITLYCKRGRKKLLYIFKQPFMRPVQTTQEEDGCSCRFPEEEEGGCELRVKFSRSADAPA | |
| | YKQGQNQLYNELNLGRREEYDVLDKRRGRDPEMGGKPRRKNPQEGLYNELQKDKMAEAYSE | |
| | IGMKGERRRGKGHDGLYQGLSTATKDTYDALHMQALPPR | |

### Bispecific CARs

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

In certain embodiments, the antibody molecule is a multi-specific (e.g., a bispecific or a trispecific) antibody molecule. Protocols for generating bispecific or heterodimeric antibody molecules are known in the art; including but not limited to, for example, the “knob in a hole” approach described in, e.g., US 5731168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono-and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also encompassed creating for bispecifc, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus futher associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. No. 5,910,573, U.S. Pat. No. 5,932,448, U.S. Pat. No. 5,959,083, U.S. Pat. No. 5,989,830, U.S. Pat. No. 6,005,079, U.S. Pat. No. 6,239,259, U.S. Pat. No. 6,294,353, U.S. Pat. No. 6,333,396, U.S. Pat. No. 6,476,198, U.S. Pat. No. 6,511,663, U.S. Pat. No. 6,670,453, U.S. Pat. No. 6,743,896, U.S. Pat. No. 6,809,185, U.S. Pat. No. 6,833,441, U.S. Pat. No. 7,129,330, U.S. Pat. No. 7,183,076, U.S. Pat. No. 7,521,056, U.S. Pat. No. 7,527,787, U.S. Pat. No. 7,534,866, U.S. Pat. No. 7,612,181, US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811A1, US2009234105A1, US2009263392A1, US2009274649A1, EP346087A2, WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO9409131A1, WO9412625A2, WO9509917A1, WO9637621A2, WO9964460A1. The contents of the above-referenced applications are incorporated herein by reference in their entireties.

Within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁-VH₁-VH₂-VL₂. Optionally, a linker is disposed between the two antibodies or antibody fragments (e.g., scFvs), e.g., between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₄-Ser)n linker, wherein n is 1, 2, 3, 4, 5, or 6, preferably 4 (SEQ ID NO: 26). In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, a bispecific CAR comprises VLs, VHs, and optionally one or more linkers in an arrangement as described herein.

In certain embodiments the antibody molecule is a bispecific antibody molecule having a first binding specificity for a first B-cell epitope and a second binding specificity for another B-cell antigen. For instance, in some embodiments the bispecific antibody molecule has a first binding specificity for CD19 and a second binding specificity for one or more of CD10, CD20, CD22, CD34, CD123, FLT-3, ROR1, CD79b, CD179b, or CD79a. In some embodiments the bispecific antibody molecule has a first binding specificity for CD19 and a second binding specificity for CD22.

### Chimeric TCR

In one aspect, the CD19 antibodies and antibody fragments of the present invention (for example, those disclosed in Tables 2 or 3) can be grafted to one or more constant domain of a T cell receptor (“TCR”) chain, for example, a TCR alpha or TCR beta chain, to create an chimeric TCR that binds specificity to CD19. Without being bound by theory, it is believed that chimeric TCRs will signal through the TCR complex upon antigen binding. For example, a CD19 scFv as disclosed herein, can be grafted to the constant domain, e.g., at least a portion of the extracellular constant domain, the transmembrane domain and the cytoplasmic domain, of a TCR chain, for example, the TCR alpha chain and/or the TCR beta chain. As another example, a CD19 antibody fragment, for example a VL domain as described herein, can be grafted to the constant domain of a TCR alpha chain, and a CD19 antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of a CD19 antibody or antibody fragment, e.g., the CDRs of a CD19 antibody or antibody fragment as described in Tables 4 or 5 may be grafted into a TCR alpha and/or beta chain to create a chimeric TCR that binds specifically to CD19. For example, the LCDRs disclosed herein may be grafted into the variable domain of a TCR alpha chain and the HCDRs disclosed herein may be grafted to the variable domain of a TCR beta chain, or vice versa. Such chimeric TCRs may be produced by methods known in the art (For example, Willemsen R A et al, Gene Therapy 2000; 7: 1369-1377; Zhang T et al, Cancer Gene Ther 2004; 11: 487-496; Aggen et al, Gene Ther. 2012 April; 19(4):365-74).

### Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR is used, e.g., in one embodiment, the transmembrane domain may be from the same protein that the signaling domain, costimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain may be derived either from a natural or from a recombinant source. Where the source is natural, the domain may be derived from any membrane-bound or transmembrane protein. In one aspect, the transmembrane domain is capable of signaling to the intracellular domain(s) whenever the CAR has bound to a target. A transmembrane domain of particular use in this invention may include at least the transmembrane domain(s) of, e.g., the alpha, beta or zeta chain of the T-cell receptor, CD28, CD3 epsilon, CD45, CD4, CDS, CD8 (e.g., CD8 alpha, CD8 beta), CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NKG2C.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8a hinge. In one embodiment, the hinge or spacer comprises (e.g., consists of) the amino acid sequence of SEQ ID NO: 2. In one aspect, the transmembrane domain comprises (e.g., consists of) a transmembrane domain of SEQ ID NO: 6.

In one aspect, the hinge or spacer comprises an IgG4 hinge. For example, in one embodiment, the hinge or spacer comprises a hinge of the amino acid sequence SEQ ID NO: 3.

In some embodiments, the hinge or spacer comprises a hinge encoded by a nucleotide sequence SEQ ID NO: 14.

In one aspect, the hinge or spacer comprises an IgD hinge. For example, in one embodiment, the hinge or spacer comprises a hinge of the amino acid sequence SEQ ID NO: 4.

In some embodiments, the hinge or spacer comprises a hinge encoded by a nucleotide sequence of SEQ ID NO: 15.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic signaling region of the CAR. A glycine-serine doublet provides a particularly suitable linker. For example, in one aspect, the linker comprises the amino acid sequence of GGGGSGGGGS (SEQ ID NO: 5). In some embodiments, the linker is encoded by a nucleotide sequence of GGTGGCGGAGGTTCTGGAGGTGGAGGTTCC (SEQ ID NO: 16).

In one aspect, the hinge or spacer comprises a KIR2DS2 hinge and portions thereof.

### Cytoplasmic Domain

The cytoplasmic domain or region of the CAR includes an intracellular signaling domain. An intracellular signaling domain is generally responsible for activation of at least one of the normal effector functions of the immune cell in which the CAR has been introducede. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary cytoplasmic signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of CD3 zeta, common FcR gamma (FCER1G), Fc gamma RIIa, FcR beta (Fc Epsilon R1b), CD3 gamma, CD3 delta, CD3 epsilon, CDS, CD22, CD79a, CD79b, CD278 (also known as “ICOS”), FcεRI, CD66d, DAP10, and DAP12. In one embodiment, a CAR of the invention comprises an intracellular signaling domain, e.g., a primary signaling domain of CD3-zeta, e.g., a CD3-zeta sequence described herein.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

Further examples of molecules containing a primary intracellular signaling domain that are of particular use in the invention include those of DAP10, DAP12, and CD32.

The intracellular domain of the CAR can comprise the CD3-zeta signaling domain by itself or it can be combined with any other desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1 (also known as PD1), ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. For example, CD27 costimulation has been demonstrated to enhance expansion, effector function, and survival of human CART cells in vitro and augments human T cell persistence and antitumor activity in vivo (Song et al. Blood. 2012; 119(3):696-706).

The intracellular signaling domains within the cytoplasmic portion of the CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling domains. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB. In one aspect, the signaling domain of 4-1BB is a signaling domain of SEQ ID NO: 7. In one aspect, the signaling domain of CD3-zeta is a signaling domain of SEQ ID NO: 9 (mutant CD3-zeta) or SEQ ID NO: 10 (wild type human CD3-zeta).

In one aspect, the intracellular is designed to comprise the signaling domain of CD3-zeta and the signaling domain of 4-1BB. In one aspect, the signaling domain of 4-1BB comprises an amino acid sequence of SEQ ID NO: 7. In one aspect, the signaling domain of 4-1BB is encoded by a nucleic acid sequence of SEQ ID NO: 18.

In one aspect, the intracellular signaling domain is designed to comprise the signaling domain of CD3-zeta and the signaling domain of CD27. In one aspect, the signaling domain of CD27 comprises an amino acid sequence of SEQ ID NO: 8. In one aspect, the signalling domain of CD27 is encoded by a nucleic acid sequence of SEQ ID NO: 19.

In one aspect, the intracellular signaling domain is designed to comprise the signaling domain of CD3-zeta and the signaling domain of CD28. In one aspect, the signaling domain of CD28 comprises an amino acid sequence of SEQ ID NO: 36. In one aspect, the signaling domain of CD28 is encoded by a nucleic acid sequence of SEQ ID NO: 37.

In one aspect, the intracellular signaling domain is designed to comprise the signaling domain of CD3-zeta and the signaling domain of ICOS. In one aspect, the signaling domain of ICOS comprises an amino acid sequence of SEQ ID NO: 38 or 43. In one aspect, the signaling domain of ICOS is encoded by a nucleic acid sequence of SEQ ID NO: 44.

### Natural Killer Cell Receptor (NKR) CARs

In an embodiment, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cyotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR molecules described herein may interact with an adaptor molecule or intracellular signaling domain, e.g., DAP12. Exemplary configurations and sequences of CAR molecules comprising NKR components are described in International Publication No. WO2014/145252, the contents of which are hereby incorporated by reference.

### Split CAR

In some embodiments, the CAR-expressing cell described herein, uses a split CAR. The split CAR approach is described in more detail in publications WO2014/055442 and WO2014/055657, incorporated herein by reference. Briefly, a split CAR system comprises a cell expressing a first CAR having a first antigen binding domain and a costimulatory domain (e.g., 4-1BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens. In embodiments the first antigen binding domain recognizes the tumor antigen or B cell antigen described herein, e.g., comprises an antigen binding domain described herein, and the second antigen binding domain recognizes a second antigen, e.g., a second tumor antigen or a second B cell antigen described herein.

Co-Expression of CAR with Other Molecules or Agents

### Co-Expression of a Second CAR

In one aspect, the CAR-expressing cell described herein can further comprise a second CAR, e.g., a second CAR that includes a different antigen binding domain, e.g., to the same target (CD19) or a different target (e.g., a target other than CD19, e.g., a B cell antigen other than CD19, e.g., CD10, CD20, CD22, CD34, CD123, FLT-3, ROR1, CD79b, CD179b, or CD79a). In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. Placement of a costimulatory signaling domain, e.g., 4-1BB, CD28, CD27, OX-40 or ICOS, onto the first CAR, and the primary signaling domain, e.g., CD3 zeta, on the second CAR can limit the CAR activity to cells where both targets are expressed. In one embodiment, the CAR expressing cell comprises a first CD19 CAR that includes a CD19 binding domain, a transmembrane domain and a costimulatory domain and a second CAR that targets an antigen other than CD19 (e.g., a target other than CD19, e.g., a B cell antigen other than CD19, e.g., CD10, CD20, CD22, CD34, CD123, FLT-3, ROR1, CD79b, CD179b, or CD79a) and includes an antigen binding domain, a transmembrane domain and a primary signaling domain. In another embodiment, the CAR expressing cell comprises a first CD19 CAR that includes a CD19 binding domain, a transmembrane domain and a primary signaling domain and a second CAR that targets an antigen other than CD19 (e.g., a target other than CD19, e.g., a B cell antigen other than CD19, e.g., CD10, CD20, CD22, CD34, CD123, FLT-3, ROR1, CD79b, CD179b, or CD79a) and includes an antigen binding domain to the antigen, a transmembrane domain and a costimulatory signaling domain.

In one embodiment, the CAR-expressing cell comprises a CD19 CAR described herein and an inhibitory CAR. In one embodiment, the inhibitory CAR comprises an antigen binding domain that binds an antigen found on normal cells but not cancer cells, e.g., normal cells that also express CD19. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, CTLA4, TIM3, LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, or TGF beta.

In one embodiment, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, e.g., as a fragment, e.g., an scFv, that does not form an association with the antigen binding domain of the second CAR, e.g., the antigen binding domain of the second CAR is a VHH.

In some embodiments, the antigen binding domain comprises a single domain antigen binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks.

In one aspect, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

According to another aspect, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

The SDAB molecules can be recombinant, CDR-grafted, humanized, camelized, de-immunized and/or in vitro generated (e.g., selected by phage display).

It has also been discovered, that cells having a plurality of chimeric membrane embedded receptors comprising an antigen binding domain that interactions between the antigen binding domain of the receptors can be undesirable, e.g., because it inhibits the ability of one or more of the antigen binding domains to bind its cognate antigen. Accordingly, disclosed herein are cells having a first and a second non-naturally occurring chimeric membrane embedded receptor comprising antigen binding domains that minimize such interactions. Also disclosed herein are nucleic acids encoding a first and a second non-naturally occurring chimeric membrane embedded receptor comprising an antigen binding domains that minimize such interactions, as well as methods of making and using such cells and nucleic acids. In an embodiment the antigen binding domain of one of the first and the second non-naturally occurring chimeric membrane embedded receptor, comprises an scFv, and the other comprises a single VH domain, e.g., a camelid, shark, or lamprey single VH domain, or a single VH domain derived from a human or mouse sequence.

In some embodiments, the claimed invention comprises a first and second CAR, wherein the antigen binding domain of one of the first and the second CAR does not comprise a variable light domain and a variable heavy domain. In some embodiments, the antigen binding domain of one of the first and the second CAR is an scFv, and the other is not an scFv. In some embodiments, the antigen binding domain of one of the first and the second CAR comprises a single VH domain, e.g., a camelid, shark, or lamprey single VH domain, or a single VH domain derived from a human or mouse sequence. In some embodiments, the antigen binding domain of one of the first and the second CAR comprises a nanobody. In some embodiments, the antigen binding domain of one of the first and the second CAR comprises a camelid VHH domain.

In some embodiments, the antigen binding domain of one of the first and the second CAR comprises an scFv, and the other comprises a single VH domain, e.g., a camelid, shark, or lamprey single VH domain, or a single VH domain derived from a human or mouse sequence. In some embodiments, the antigen binding domain of one of the first and the second CAR comprises an scFv, and the other comprises a nanobody. In some embodiments, the antigen binding domain of one of the first and the second CAR comprises comprises an scFv, and the other comprises a camelid VHH domain.

In some embodiments, when present on the surface of a cell, binding of the antigen binding domain of the first CAR to its cognate antigen is not substantially reduced by the presence of the second CAR. In some embodiments, binding of the antigen binding domain of the first CAR to its cognate antigen in the presence of the second CAR is 85%, 90%, 95%, 96%, 97%, 98% or 99% of binding of the antigen binding domain of the first CAR to its cognate antigen in the absence of the second CAR.

In some embodiments, when present on the surface of a cell, the antigen binding domains of the first and the second CAR, associate with one another less than if both were scFv antigen binding domains. In some embodiments, the antigen binding domains of the first and the second CAR, associate with one another 85%, 90%, 95%, 96%, 97%, 98% or 99% less than if both were scFv antigen binding domains.

Co-Expression of an Agent that Enhances CAR Activity

In another aspect, the CAR-expressing cell described herein can further express another agent, e.g., an agent that enhances the activity or fitness of a CAR-expressing cell.

For example, in one embodiment, the agent can be an agent which inhibits a molecule that modulates or regulates, e.g., inhibits, T cell function. In some embodiments, the molecule that modulates or regulates T cell function is an inhibitory molecule. Inhibitory molecules, e.g., PD-1, can, in some embodiments, decrease the ability of a CAR-expressing cell to mount an immune effector response. Examples of inhibitory molecules include PD-1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta.

In embodiments, an agent, e.g., an inhibitory nucleic acid, e.g., a dsRNA, e.g., an siRNA or shRNA; or e.g., an inhibitory protein or system, e.g., a clustered regularly interspaced short palindromic repeats (CRISPR), a transcription-activator like effector nuclease (TALEN), or a zinc finger endonuclease (ZFN), e.g., as described herein, can be used to inhibit expression of a molecule that modulates or regulates, e.g., inhibits, T-cell function in the CAR-expressing cell. In an embodiment the agent is an shRNA, e.g., an shRNA described herein. In an embodiment, the agent that modulates or regulates, e.g., inhibits, T-cell function is inhibited within a CAR-expressing cell. For example, a dsRNA molecule that inhibits expression of a molecule that modulates or regulates, e.g., inhibits, T-cell function is linked to the nucleic acid that encodes a component, e.g., all of the components, of the CAR.

In one embodiment, the agent that inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD-1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). In one embodiment, the agent comprises a first polypeptide of PD-1 or a fragment thereof (e.g., at least a portion of an extracellular domain of PD-1), and a second polypeptide of an intracellular signaling domain described herein (e.g., a CD28 signaling domain described herein and/or a CD3 zeta signaling domain described herein). PD-1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD-1, PD-L1 and PD-L2 have been shown to downregulate T cell activation upon binding to PD-1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD-1 with PD-L1.

In one embodiment, the agent comprising the extracellular domain (ECD) of an inhibitory molecule, e.g., Programmed Death 1 (PD-1), can be fused to a transmembrane domain and intracellular signaling domains such as 4-1BB and CD3 zeta (also referred to herein as a PD1 CAR). In one embodiment, the PD1 CAR, when used in combinations with a CD19 CAR described herein, improves the persistence of the T cell. In one embodiment, the CAR is a PD1 CAR comprising the extracellular domain of PD-1 indicated as underlined in SEQ ID NO: 24 and a signal sequence at amino acids 1-21 of SEQ ID NO: 24. In one embodiment, the PD1 CAR comprises the amino acid sequence of SEQ ID NO: 24.

In one embodiment, the PD1 CAR without the N-terminal signal sequence comprises the amino acid sequence provided of SEQ ID NO: 22.

In one embodiment, the agent comprises a nucleic acid sequence encoding the PD1 CAR with the N-terminal signal sequence, e.g., the PD1 CAR described herein. In one embodiment, the nucleic acid sequence for the PD1 CAR is shown in Table 1, with the PD1 ECD underlined in SEQ ID NO: 23.

In another example, in one embodiment, the agent which enhances the activity of a CAR-expressing cell can be a costimulatory molecule or costimulatory molecule ligand. Examples of costimulatory molecules include MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83., e.g., as described herein. Examples of costimulatory molecule ligands include CD80, CD86, CD4OL, ICOSL, CD70, OX4OL, 4-1BBL, GITRL, and LIGHT. In embodiments, the costimulatory molecule ligand is a ligand for a costimulatory molecule different from the costimulatory molecule domain of the CAR. In embodiments, the costimulatory molecule ligand is a ligand for a costimulatory molecule that is the same as the costimulatory molecule domain of the CAR. In an embodiment, the costimulatory molecule ligand is 4-1BBL. In an embodiment, the costimulatory ligand is CD80 or CD86. In an embodiment, the costimulatory molecule ligand is CD70. In embodiments, a CAR-expressing immune effector cell described herein can be further engineered to express one or more additional costimulatory molecules or costimulatory molecule ligands.

Co-Expression of CAR with a Chemokine Receptor

In embodiments, the CAR-expressing cell described herein, e.g., CD19 CAR-expressing cell, further comprises a chemokine receptor molecule. Transgenic expression of chemokine receptors CCR2b or CXCR2 in T cells enhances trafficking to CCL2- or CXCL1-secreting solid tumors including melanoma and neuroblastoma (Craddock et al., J Immunother. 2010 October; 33(8):780-8 and Kershaw et al., Hum Gene Ther. 2002 Nov. 1; 13(16):1971-80). Thus, without wishing to be bound by theory, it is believed that chemokine receptors expressed in CAR-expressing cells that recognize chemokines secreted by tumors, e.g., solid tumors, can improve homing of the CAR-expressing cell to the tumor, facilitate the infiltration of the CAR-expressing cell to the tumor, and enhances antitumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell (e.g., CAR-Tx) described herein include a CXC chemokine receptor (e.g., CXCR1, CXCR2, CXCR3, CXCR4, CXCRS, CXCR6, or CXCR7), a CC chemokine receptor (e.g., CCR1, CCR2, CCR3, CCR4, CCRS, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof. In one embodiment, the chemokine receptor molecule to be expressed with a CAR described herein is selected based on the chemokine(s) secreted by the tumor. In one embodiment, the CAR-expressing cell described herein further comprises, e.g., expresses, a CCR2b receptor or a CXCR2 receptor. In an embodiment, the CAR described herein and the chemokine receptor molecule are on the same vector or are on two different vectors. In embodiments where the CAR described herein and the chemokine receptor molecule are on the same vector, the CAR and the chemokine receptor molecule are each under control of two different promoters or are under the control of the same promoter.

### Nucleic Acid Constructs Encoding a CAR

The present invention provides CAR transgenes comprising nucleic acid sequences encoding one or more CAR constructs of the invention. In one aspect, the CAR transgene is provided as a messenger RNA transcript. In one aspect, the CAR transgene is provided as a DNA construct.

Accordingly, in one aspect, the invention pertains to an isolated nucleic acid molecule encoding a chimeric antigen receptor (CAR), wherein the CAR comprises an anti-CD19 binding domain (e.g., a murine anti-CD19 binding domain or humanized anti-CD19 binding domain), a transmembrane domain, and an intracellular signaling domain comprising a stimulatory domain. In one embodiment, the anti-CD19 binding domain is an anti-CD19 binding domain described herein, e.g., an anti-CD19 binding domain which comprises a sequence selected from a group consisting of SEQ ID NO: 45-56, 69-80, 106, 109, 110, 112, or 115, or a sequence with 95-99% identify thereof. In one embodiment, the isolated nucleic acid molecule further comprises a sequence encoding a costimulatory domain. In one embodiment, the transmembrane domain is a transmembrane domain of a protein selected from the group consisting of the alpha, beta or zeta chain of the T-cell receptor, CD28, CD3 epsilon, CD45, CD4, CDS, CD8, CD9, CD16, CD22, CD33, CD37, CD64, CD80, CD86, CD134, CD137 and CD154. In one embodiment, the transmembrane domain comprises a sequence of SEQ ID NO: 6, or a sequence with 95-99% identity thereof. In one embodiment, the anti-CD19 binding domain is connected to the transmembrane domain by a hinge region, e.g., a hinge described herein. In one embodiment, the hinge region comprises SEQ ID NO: 2, SEQ ID NO: 3, SEQ ID NO: 4, SEQ ID NO: 5, SEQ ID NO: 16, or SEQ ID NO: 39, or a sequence with 95-99% identity thereof. In one embodiment, the isolated nucleic acid molecule further comprises a sequence encoding a costimulatory domain. In one embodiment, the costimulatory domain is a functional signaling domain of a protein selected from the group consisting of OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, and PAG/Cbp. In one embodiment, the costimulatory domain comprises a sequence of SEQ ID NO: 7, or a sequence with 95-99% identity thereof. In one embodiment, the intracellular signaling domain comprises a functional signaling domain of 4-1BB and a functional signaling domain of CD3 zeta. In one embodiment, the intracellular signaling domain comprises the sequence of SEQ ID NO: 7 or SEQ ID NO: 8, or a sequence with 95-99% identity thereof, and the sequence of SEQ ID NO: 9 or SEQ ID NO: 10, or a sequence with 95-99% identity thereof, wherein the sequences comprising the intracellular signaling domain are expressed in the same frame and as a single polypeptide chain. In another aspect, the invention pertains to an isolated nucleic acid molecule encoding a CAR construct comprising a leader sequence of SEQ ID NO: 1, a scFv domain having a sequence selected from the group consisting of SEQ ID NO: 45; SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 106, SEQ ID NO: 109, SEQ ID NO: 110, SEQ ID NO: 112, and SEQ ID NO: 115 (or a sequence with 95-99% identify thereof), a hinge region of SEQ ID NO: 2, SEQ ID NO:3, SEQ ID NO:4, SEQ ID NO:5, SEQ ID NO: 16, or SEQ ID NO: 39 (or a sequence with 95-99% identity thereof), a transmembrane domain having a sequence of SEQ ID NO: 6 (or a sequence with 95-99% identity thereof), a 4-1BB costimulatory domain having a sequence of SEQ ID NO: 7 (or a sequence with 95-99% identity thereof) or a CD27 costimulatory domain having a sequence of SEQ ID NO: 8 (or a sequence with 95-99% identity thereof), and a CD3 zeta stimulatory domain having a sequence of SEQ ID NO: 9 or SEQ ID NO: 10 (or a sequence with 95-99% identity thereof).

In another aspect, the invention pertains to an isolated polypeptide molecule encoded by the nucleic acid molecule. In one embodiment, the isolated polypeptide molecule comprises a sequence selected from the group consisting of SEQ ID NO: 93; SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 108, SEQ ID NO: 111, SEQ ID NO: 113, SEQ ID NO: 114, and SEQ ID NO: 116, or a sequence with 95-99% identity thereof.

In another aspect, the invention pertains to an isolated nucleic acid molecule encoding a chimeric antigen receptor (CAR) molecule that comprises an anti-CD19 binding domain, a transmembrane domain, and an intracellular signaling domain comprising a stimulatory domain, and wherein the nucleic acid encoding the anti-CD19 binding domain comprises a sequence selected from the group consisting of SEQ ID NO: 57; SEQ ID NO: 58, SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, and SEQ ID NO: 105, ora sequence with 95-99% identify thereof.

In one embodiment, the encoded CAR molecule further comprises a sequence encoding a costimulatory domain. In one embodiment, the costimulatory domain is a functional signaling domain of a protein selected from the group consisting of OX40, CD27, CD28, CDS, ICAM-1, LFA-1 (CD11a/CD18) and 4-1BB (CD137). In one embodiment, the costimulatory domain comprises a sequence of SEQ ID NO: 7. In one embodiment, the transmembrane domain is a transmembrane domain of a protein selected from the group consisting of the alpha, beta or zeta chain of the T-cell receptor, CD28, CD3 epsilon, CD45, CD4, CDS, CD8, CD9, CD16, CD22, CD33, CD37, CD64, CD80, CD86, CD134, CD137 and CD154. In one embodiment, the transmembrane domain comprises a sequence of SEQ ID NO: 6. In one embodiment, the intracellular signaling domain comprises a functional signaling domain of 4-1BB and a functional signaling domain of zeta. In one embodiment, the intracellular signaling domain comprises the sequence of SEQ ID NO: 7 and the sequence of SEQ ID NO: 9, wherein the sequences comprising the intracellular signaling domain are expressed in the same frame and as a single polypeptide chain. In one embodiment, the anti-CD19 binding domain is connected to the transmembrane domain by a hinge region. In one embodiment, the hinge region comprises SEQ ID NO: 2. In one embodiment, the hinge region comprises SEQ ID NO: 3, SEQ ID NO: 4, SEQ ID NO: 5, SEQ ID NO: 16, or SEQ ID NO: 39.

In another aspect, the invention pertains to an isolated CAR molecule comprising a leader sequence of SEQ ID NO: 1, a scFv domain having a sequence selected from the group consisting of SEQ ID NOS: 45-56, 109, 110, 112, and 115, or a sequence with 95-99% identify thereof, a hinge region of SEQ ID NO:2, SEQ ID NO: 3, SEQ ID NO: 4, SEQ ID NO: 5, SEQ ID NO: 16, or SEQ ID NO: 39, a transmembrane domain having a sequence of SEQ ID NO: 6, a 4-1BB costimulatory domain having a sequence of SEQ ID NO: 7 or a CD27 costimulatory domain having a sequence of SEQ ID NO: 8, and a CD3 zeta stimulatory domain having a sequence of SEQ ID NO: 9 or SEQ ID NO: 10. In one embodiment, the encoded CAR molecule comprises a sequence selected from the group consisting of SEQ ID NOS: 93-104, 108, 111, 114, 116, or a sequence with 95-99% identify thereof.

The present invention further provides vectors comprising CAR transgenes. In one aspect, a CAR vectors can be directly transduced into a cell, e.g., a T cell or NK cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the CAR construct in mammalian T cells or NK cells. In one aspect, the mammalian T cell is a human T cell or a human NK cell.

The present invention also includes a CAR encoding RNA construct that can be directly transfected into a cell, e.g., a T cell or a NK cell. A method for generating mRNA for use in transfection involves in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the gene to be expressed, and a polyA tail, typically 50-2000 bases in length (SEQ ID NO: 35). RNA so produced can efficiently transfect different kinds of cells. In one aspect, the template includes sequences for the CAR.

In one aspect the CAR (e.g., CD19 CAR) transgene is encoded by a messenger RNA (mRNA). In one aspect the mRNA encoding the CAR transgene is introduced into a T cell for production of a CART cell, or a NK cell.

### Vectors

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In one embodiment, the vector comprising the nucleic acid encoding the desired CAR of the invention is a DNA, a RNA, a plasmid, an adenoviral vector, a lentivirus vector, or a retrovirus vector. A retroviral vector may also be, e.g., a gammaretroviral vector. A gammaretroviral vector may include, e.g., a promoter, a packaging signal (ψ), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the invention is an adenoviral vector (A5/35). In another embodiment, the expression of nucleic acids encoding CARs can be accomplished using of transposons such as sleeping beauty, CRISPR, CAS9, and zinc finger nucleases. See, e.g., June et al. 2009 Nature Reviews Immunology 9.10: 704-716, incorporated herein by reference in its entirety.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1α, ubiquitin C, or phosphoglycerokinase (PGK) promoters.

An example of a promoter that is capable of expressing a CAR transgene in a mammalian T cell is the EF1alpha promoter (EF1a or EF1α). The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from transgenes cloned into a lentiviral vector. See, e.g., Milone et al., Mol. Ther. 17(8): 1453-1464 (2009). In one aspect, the EF1a promoter comprises the sequence provided as SEQ ID NO:11.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired. The nucleotide sequences of exemplary PGK promoters are provided as wild type PGK promoter in SEQ ID NO: 126, or truncated version of the PGK promoter, e.g., PGK100 as provided in SEQ ID NO: 127, PGK200 as provided in SEQ ID NO: 128, PGK300 as provided in SEQ ID NO: 129, and PGK400 as provided in SEQ ID NO: 130.

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In one embodiment, the vector can further comprise a nucleic acid encoding a second CAR. In one embodiment, the second CAR includes an antigen binding domain to, e.g., a target other than CD19 (e.g., a B cell antigen other than CD19, e.g., CD10, CD20, CD22, CD34, CD123, FLT-3, ROR1, CD79b, CD179b, or CD79a). In one embodiment, the vector comprises a nucleic acid sequence encoding a first CAR that targets a first antigen and includes an intracellular signaling domain having a costimulatory signaling domain but not a primary signaling domain, and a nucleic acid encoding a second CAR that targets a second, different, antigen and includes an intracellular signaling domain having a primary signaling domain but not a costimulatory signaling domain. In one embodiment, the vector comprises a nucleic acid encoding a first CD19 CAR that includes a CD19 binding domain, a transmembrane domain and a costimulatory domain and a nucleic acid encoding a second CAR that targets an antigen other than CD19 (e.g., a B cell antigen other than CD19, e.g., CD10, CD20, CD22, CD34, CD123, FLT-3, ROR1, CD79b, CD179b, or CD79a) and includes an antigen binding domain, a transmembrane domain and a primary signaling domain. In another embodiment, the vector comprises a nucleic acid encoding a first CD19 CAR that includes a CD19 binding domain, a transmembrane domain and a primary signaling domain and a nucleic acid encoding a second CAR that targets an antigen other than CD19 (e.g., a B cell antigen other than CD19, e.g., CD10, CD20, CD22, CD34, CD123, FLT-3, ROR1, CD79b, CD179b, or CD79a) and includes an antigen binding domain to the antigen, a transmembrane domain and a costimulatory signaling domain.

In one embodiment, the vector comprises a nucleic acid encoding a CAR (e.g., CD19 CAR) described herein and a nucleic acid encoding an inhibitory CAR. In one embodiment, the inhibitory CAR comprises an antigen binding domain that binds an antigen found on normal cells but not cancer cells, e.g., normal cells that also express CD19. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, CTLA4, TIM3, CEACAM (e.g., CEACAM-1, CEACAM-3 and/or CEACAM-5), LAGS, VISTA, BTLA, TIGIT, LAIR1, CD160, 2B4 and/or TGF beta.

In embodiments, the vector may comprise two or more nucleic acid sequences, wherein one of the nucleic acid sequences encodes a CAR described herein, e.g., a CD19 CAR described herein. In one embodiment, the other nucleic acid can encode a second CAR, e.g., an inhibitory CAR or a specifically binds to an antigen other than CD19 (e.g., a B cell antigen other than CD19, e.g., CD10, CD20, CD22, CD34, CD123, FLT-3, ROR1, CD79b, CD179b, or CD79a), or a polypeptide that can regulate activity of the CAR (e.g., CD19 CAR) described herein. In such embodiments, the two or more nucleic acid sequences, e.g., encoding a CAR (e.g., CD19 CAR) described herein and a second CAR or other polypeptide, are encoded by a single nucleic molecule in the same frame and as a single polypeptide chain. In one embodiment, the two or more polypeptides can be separated by one or more peptide cleavage sites (e.g., an auto-cleavage site or a substrate for an intracellular protease). Examples of peptide cleavage sites include the following, wherein the GSG residues are optional:T2A as provided in SEQ ID NO: 131, P2A as provided in SEQ ID NO: 132, E2A as provided in SEQ ID NO: 133, and F2A as provided in SEQ ID NO: 134.

Methods of introducing and expressing genes into a cell are known in the art. In the context of an expression vector, the vector can be readily introduced into a host cell, e.g., mammalian, bacterial, yeast, or insect cell by any method in the art and are described in pages 208-210 of International Application WO 2016/164731, filed Apr. 8, 2016, which is incorporated by reference in its entirety.

The present invention further provides a vector comprising a CAR encoding nucleic acid molecule. In one aspect, a CAR vector can be directly transduced into a cell, e.g., a T cell or a NK cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the CAR construct in mammalian T cells. In one aspect, the mammalian T cell is a human T cell. In one aspect, the mammalian cell is a human NK cell.

### RNA Transfection

Disclosed herein are methods for producing an in vitro transcribed RNA CAR. The present invention also includes a CAR encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length (SEQ ID NO:35). RNA so produced can efficiently transfect different kinds of cells. In one aspect, the template includes sequences for the CAR.

In one aspect the CAR (e.g., CD19 CAR) is encoded by a messenger RNA (mRNA). In one aspect the mRNA encoding the CAR is introduced into an immune effector cells, e.g., a T cell or a NK cell, for production of a CAR-expressing cell, e.g., a CART cell or a CAR NK cell. Additional methods of RNA transfection are described on pages 192-196 of International Application WO 2016/164731, filed Apr. 8, 2016, which is incorporated by reference in its entirety.

### Non-Viral Delivery Methods

In some aspects, non-viral methods can be used to deliver a nucleic acid encoding a CAR described herein into a cell or tissue or a subject. In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. Additional and exemplary transposons and non-viral delivery methods are described on pages 196-198 of International Application WO 2016/164731, filed Apr. 8, 2016, which is incorporated by reference in its entirety.

### Sources of Cells

Prior to expansion and genetic modification, e.g., to express a CAR described herein, a source of cells, e.g., T cell or NK cells, can be obtained from a subject. The term “subject” is intended to include living organisms in which an immune response can be elicited (e.g., mammals). Examples of subjects include humans, dogs, cats, mice, rats, and transgenic species thereof.

In embodiments, immune effector cells (e.g., a population of immune effector cells), e.g., T cells, are derived from (e.g., differentiated from) a stem cell, e.g., an embryonic stem cell or a pluripotent stem cell, e.g., an induced pluripotent stem cell (iPSC). In embodiments, the cells are autologous or allogeneic. In embodiments wherein the cells are allogeneic, the cells, e.g., derived from stem cells (e.g., iPSCs), are modified to reduce their alloreactivity. For example, the cells can be modified to reduce alloreactivity, e.g., by modifying (e.g., disrupting) their T cell receptor. In embodiments, a site specific nuclease can be used to disrupt the T cell receptor, e.g., after T-cell differentiation. In other examples, cells, e.g., T cells derived from iPSCs, can be generated from virus-specific T cells, which are less likely to cause graft-versus-host disease because of their recognition of a pathogen-derived antigen. In yet other examples, alloreactivity can be reduced, e.g., minimized, by generating iPSCs from common HLA haplotypes such that they are histocompatible with matched, unrelated recipient subjects. In yet other examples, alloreactivity can be reduced, e.g., minimized, by repressing HLA expression through genetic modification. For example, T cells derived from iPSCs can be processed as described in, e.g., Themeli et al. Nat. Biotechnol. 31.10(2013):928-35, incorporated herein by reference. In some examples, immune effector cells, e.g., T cells, derived from stem cells, can be processed/generated using methods described in WO2014/165707, incorporated herein by reference. Additional embodiments pertaining to allogeneic cells are described herein, e.g., in the “Allogeneic CAR Immune Effector Cells” section herein.

T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain aspects of the present disclosure, any number of T cell lines available in the art, may be used. In certain aspects of the present disclosure, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the invention, the cells are washed with phosphate buffered saline (PBS). In an alternative aspect, the wash solution lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one aspect, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+T cells, can be further isolated by positive or negative selection techniques. For example, in one aspect, T cells are isolated by incubation with anti-CD3/anti-CD28 (e.g., 3×28)-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is 1e7 cells to 20 uL, or 1e7 cells to15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In an embodiment, a subject is pre-treated with one or more therapies that reduce T_{REG} cells prior to collection of cells for CAR-expressing cell product manufacturing, thereby reducing the risk of subject relapse to CAR-expressing cell treatment. In an embodiment, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In an embodiment, a subject is pre-treated with cyclophosphamide prior to collection of cells for CAR-expressing cell product manufacturing, thereby reducing the risk of subject relapse to CAR-expressing cell treatment. In an embodiment, a subject is pre-treated with an anti-GITR antibody prior to collection of cells for CAR-expressing cell product manufacturing, thereby reducing the risk of subject relapse to CAR-expressing cell treatment.

In one embodiment, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include B7-H1, B7-1, CD160, P1H, 2B4, PD1, TIM3, CEACAM (e.g., CEACAM-1, CEACAM-3 and/or CEACAM-5), LAG3, TIGIT, CTLA-4, BTLA and LAIR1. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/ml is used. In one aspect, a concentration of 1 billion cells/ml is used. In a further aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10e6/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present disclosure.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

### Allogeneic CAR Immune Effector Cells

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II.

A T cell lacking a functional TCR can be, e.g., engineered such that it does not express any functional TCR on its surface, engineered such that it does not express one or more subunits that comprise a functional TCR or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class I and/or HLA class II, is downregulated.

In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not expresses or expresses at low levels an inhibitory molecule, e.g. by any method described herein. For example, the cell can be a cell that does not express or expresses at low levels an inhibitory molecule, e.g., that can decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta. Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell. Expression systems for siRNA and shRNAs, and exemplary shRNAs, are described, e.g., in paragraphs 649 and 650 of International Publication WO2015/142675, filed Mar. 13, 2015, which is incorporated by reference in its entirety.

### CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein.

A “CRISPR/Cas” system refers to a system derived from CRISPR and Cas which can be used to silence or mutate a TCR and/or HLA gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

The CRISPR/Cas system, and uses thereof, are described, e.g., in paragraphs 651-658 of International Publication WO2015/142675, filed Mar. 13, 2015, which is incorporated by reference in its entirety.

TALEN to Inhibit TCR and/or HLA

TALEN″ or “TALEN to HLA and/or TCR” or “TALEN to inhibit HLA and/or TCR” refers to a transcription activator-like effector nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

TALENs, and uses thereof, are described, e.g., in paragraphs 659-665 of International Publication WO2015/142675, filed Mar. 13, 2015, which is incorporated by reference in its entirety.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF beta), in a cell, e.g., T cell.

ZFNs, and uses thereof, are described, e.g., in paragraphs 666-671 of International Publication WO2015/142675, filed Mar. 13, 2015, which is incorporated by reference in its entirety.

### Telomerase Expression

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in an embodiment, an immune effector cell, e.g., a T cell, ectopically expresses a telomerase subunit, e.g., the catalytic subunit of telomerase, e.g., TERT, e.g., hTERT. In some aspects, this disclosure provides a method of producing a CAR-expressing cell, comprising contacting a cell with a nucleic acid encoding a telomerase subunit, e.g., the catalytic subunit of telomerase, e.g., TERT, e.g., hTERT. The cell may be contacted with the nucleic acid before, simultaneous with, or after being contacted with a construct encoding a CAR.

In one aspect, the disclosure features a method of making a population of immune effector cells (e.g., T cells, NK cells). In an embodiment, the method comprises: providing a population of immune effector cells (e.g., T cells or NK cells), contacting the population of immune effector cells with a nucleic acid encoding a CAR; and contacting the population of immune effector cells with a nucleic acid encoding a telomerase subunit, e.g., hTERT, under conditions that allow for CAR and telomerase expression. In an embodiment, the nucleic acid encoding the telomerase subunit is DNA. In an embodiment, the nucleic acid encoding the telomerase subunit comprises a promoter capable of driving expression of the telomerase subunit.

In an embodiment, hTERT has the amino acid sequence of GenBank Protein ID AAC51724.1 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795) as provided in SEQ ID NO: 135.

In an embodiment, the hTERT has a sequence at least 80%, 85%, 90%, 95%, 96%, 97%, 98%, or 99% identical to the sequence of SEQ ID NO: 135. In an embodiment, the hTERT has a sequence of SEQ ID NO: 135. In an embodiment, the hTERT comprises a deletion (e.g., of no more than 5, 10, 15, 20, or 30 amino acids) at the N-terminus, the C-terminus, or both. In an embodiment, the hTERT comprises a transgenic amino acid sequence (e.g., of no more than 5, 10, 15, 20, or 30 amino acids) at the N-terminus, the C-terminus, or both.

In an embodiment, the hTERT is encoded by the nucleic acid sequence of GenBank Accession No. AF018167 (Meyerson et al., “hEST2, the Putative Human Telomerase Catalytic Subunit Gene, Is Up-Regulated in Tumor Cells and during Immortalization” Cell Volume 90, Issue 4, 22 August 1997, Pages 785-795) as provided in SEQ ID NO: 136

In an embodiment, the hTERT is encoded by a nucleic acid having a sequence at least 80%, 85%, 90%, 95%, 96, 97%, 98%, or 99% identical to the sequence of SEQ ID NO: 136. In an embodiment, the hTERT is encoded by a nucleic acid of SEQ ID NO: 136.

### Activation and Expansion of Immune Effector Cells (e.g., T Cells)

Immune effector cells, such as T cells, may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, a population of immune effector cells, e.g., T cells may be expanded by contact with a surface having attached thereto an agent that stimulates a CD3/TCR complex associated signal and a ligand that stimulates a costimulatory molecule on the surface of the immune effector cells, e.g., T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present disclosure.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present disclosure, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect of the present disclosure, more anti-CD28 antibody is bound to the particles than anti-CD3 antibody, i.e., the ratio of CD3:CD28 is less than one. In certain aspects of the invention, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one preferred aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one aspect, a ratio of particles to cells of 1:1 or less is used. In one particular aspect, a preferred particle: cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present disclosure. In particular, ratios will vary depending on particle size and on cell size and type. In one aspect, the most typical ratios for use are in the neighborhood of 1:1, 2:1 and 3:1 on the first day.

In further aspects of the present disclosure, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one aspect the cells (for example, 10⁴ to 10⁹ T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present disclosure. In certain aspects, it may be desirable to significantly decrease the volume in which particles and cells are mixed together (i.e., increase the concentration of cells), to ensure maximum contact of cells and particles. For example, in one aspect, a concentration of about 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one embodiment, cells transduced with a nucleic acid encoding a CAR, e.g., a CAR described herein, are expanded, e.g., by a method described herein. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. In one embodiment, the cells are expanded for a period of 8 days or less, e.g., 7, 6 or 5 days. In one embodiment, the cells, e.g., a CAR-expressing cell described herein, are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In one embodiment, the cells, e.g., a CAR-expressing cell described herein, expanded for 5 days show at least a one, two, three or four fold increase in cells doublings upon antigen stimulation as compared to the same cells expanded in culture for 9 days under the same culture conditions. In one embodiment, the cells, e.g., the cells expressing a CAR described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In one embodiment, the cells, e.g., a CAR-expressing cell described herein, expanded for 5 days show at least a one, two, three, four, five, tenfold or more increase in pg/ml of proinflammatory cytokine production, e.g., IFN-γ and/or GM-CSF levels, as compared to the same cells expanded in culture for 9 days under the same culture conditions.

In one aspect of the present disclosure, the mixture may be cultured for several hours (about 3 hours) to about 14 days or any hourly integer value in between. In one aspect, the mixture may be cultured for 21 days. In one aspect of the invention the beads and the T cells are cultured together for about eight days. In one aspect, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In one embodiment, the cells are expanded in an appropriate media (e.g., media described herein) that includes one or more interleukin that result in at least a 200-fold (e.g., 200-fold, 250-fold, 300-fold, 350-fold) increase in cells over a 14 day expansion period, e.g., as measured by a method described herein such as flow cytometry. In one embodiment, the cells are expanded in the presence IL-15 and/or IL-7 (e.g., IL-15 and IL-7).

In an embodiment, a method of expanding the cells (e.g., CAR-expressing cells, e.g., CD19 CAR-expressing cells, e.g., CD19 CAR-expressing cells described herein, e.g., CTL-019) described herein (e.g., ex vivo expansion) comprises contacting the cells with a PD-1 inhibitor, e.g., PD-1 inhibitor described herein, e.g., anti-PD-1 antibody molecule described herein, e.g., PDR-001.

In embodiments, methods described herein, e.g., CAR-expressing cell manufacturing methods, comprise removing T regulatory cells, e.g., CD25+ T cells, from a cell population, e.g., using an anti-CD25 antibody, or fragment thereof, or a CD25-binding ligand, IL-2. Methods of removing T regulatory cells, e.g., CD25+ T cells, from a cell population are described herein. In embodiments, the methods, e.g., manufacturing methods, further comprise contacting a cell population (e.g., a cell population in which T regulatory cells, such as CD25+ T cells, have been depleted; or a cell population that has previously contacted an anti-CD25 antibody, fragment thereof, or CD25-binding ligand) with IL-15 and/or IL-7. For example, the cell population (e.g., that has previously contacted an anti-CD25 antibody, fragment thereof, or CD25-binding ligand) is expanded in the presence of IL-15 and/or IL-7.

In an embodiment, methods described herein, e.g., CAR-expressing cell manufacturing methods, comprise contacting the cells (e.g., CAR-expressing cells, e.g., CD19 CAR-expressing cells, e.g., CD19 CAR-expressing cells described herein, e.g., CTL-019) with a PD-1 inhibitor, e.g., PD-1 inhibitor described herein, e.g., anti-PD-1 antibody molecule described herein, e.g., PDR-001.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15, during the manufacturing of the CAR-expressing cell, e.g., ex vivo. In embodiments, a CAR-expressing cell described herein is contacted with a composition comprising a IL-15 polypeptide during the manufacturing of the CAR-expressing cell, e.g., ex vivo. In embodiments, a CAR-expressing cell described herein is contacted with a composition comprising a combination of both a IL-15 polypeptide and a IL-15 Ra polypeptide during the manufacturing of the CAR-expressing cell, e.g., ex vivo. In embodiments, a CAR-expressing cell described herein is contacted with a composition comprising hetIL-15 during the manufacturing of the CAR-expressing cell, e.g., ex vivo.

In one embodiment the CAR-expressing cell described herein is contacted with a composition comprising hetIL-15 during ex vivo expansion. In an embodiment, the CAR-expressing cell described herein is contacted with a composition comprising an IL-15 polypeptide during ex vivo expansion. In an embodiment, the CAR-expressing cell described herein is contacted with a composition comprising both an IL-15 polypeptide and an IL-15Ra polypeptide during ex vivo expansion. In one embodiment the contacting results in the survival and proliferation of a lymphocyte subpopulation, e.g., CD8+ T cells.

In one embodiment, the cells are cultured (e.g., expanded, simulated, and/or transduced) in media comprising serum. The serum may be, e.g., human AB serum (hAB). In some embodiments, the hAB serum is present at about 2%, about 5%, about 2-3%, about 3-4%, about 4-5%, or about 2-5%. 2% and 5% serum are each suitable levels that allow for many fold expansion of T cells. Furthermore, as shown in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31, medium containing 2% human AB serum is suitable for ex vivo expansion of T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

In some embodiments, cells transduced with a nucleic acid encoding a CAR, e.g., a CAR described herein, can be selected for administration based upon, e.g., protein expression levels of one or more of CCL20, GM-CSF, IFNγ, IL-10, IL-13, IL-17a, IL-2, IL-21, IL-4, IL-5, IL-6, IL-9, TNFα and/or combinations thereof. In some embodiments, cells transduced with a nucleic acid encoding a CAR, e.g., a CAR described herein, can be selected for administration based upon, e.g., protein expression levels of CCL20, IL-17a, IL-6 and combinations thereof.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR, e.g., CD19 CAR, is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR, e.g., CD19 CAR, are described, e.g., in paragraphs [0417]-[00423] of International Publication WO2015/090230, filed Dec. 19, 2014, which is incorporated by reference in its entirety.

### Populations of CAR Cells

In another aspect, the present invention provides a population of CAR-expressing cells, e.g., a population of CD19 CAR-expressing cells. In some embodiments, the population of CAR-expressing cells comprises a mixture of cells expressing different CARs.

For example, in one embodiment, the population of CAR-expressing cells can include a first cell expressing a CAR having an anti-CD19 binding domain described herein, and a second cell expressing a CAR having a different anti-CD19 binding domain, e.g., an anti-CD19 binding domain described herein that differs from the anti-CD19 binding domain in the CAR expressed by the first cell.

As another example, the population of CAR-expressing cells can include a first cell expressing a CAR that includes an anti-CD19 binding domain, e.g., as described herein, and a second cell expressing a CAR that includes an antigen binding domain to a target other than CD19 (e.g., a B cell antigen other than CD19, e.g., CD10, CD20, CD22, CD34, CD123, FLT-3, ROR1, CD79b, CD179b, or CD79a). In one embodiment, the population of CAR-expressing cells includes, e.g., a first cell expressing a CAR that includes a primary intracellular signaling domain, and a second cell expressing a CAR that includes a secondary signaling domain.

In one embodiment, the population of CAR-expressing cells can include a first cell expressing a CAR that includes an anti-CD19 binding domain and a second cell expressing a CAR that includes an antigen binding domain that targets, e.g., specifically binds, an antigen expressed on B cells, or a B cell antigen. In one embodiment, the B cell antigen is CD19, e.g., where the first cell and the second cell express different CD19 CARs. In another embodiment, the B cell antigen is an antigen other than CD19, e.g., CD10, CD20, CD22, CD34, CD123, FLT-3, ROR1, CD79b, CD179b, or CD79a.

In another aspect, the present invention provides a population of cells wherein at least one cell in the population expresses a CAR having an anti-CD19 binding domain described herein, and a second cell expressing another agent, e.g., an agent which enhances the activity or function of a CAR-expressing cell. For example, in one embodiment, the agent can be an agent which modulates or regulates, e.g., inhibits, T cell function. In some embodiments, the molecule that modulates or regulates T cell function is an inhibitory molecule, e.g., an agent described herein. Inhibitory molecules, e.g., can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, PD-L1, CTLA4, TIM3, LAG3, VISTA, BTLA, TIGIT, LAlR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta, or a fragment of any of these (e.g., at least a portion of an extracellular domain of any of these), and a second polypeptide which is an intracellular signaling domain described herein (e.g., comprising a costimulatory domain (e.g., 4-1BB, CD27, CD28, or ICOS, e.g., as described herein) and/or a primary signaling domain (e.g., a CD3 zeta signaling domain described herein). In one embodiment, the agent comprises a first polypeptide of PD1 or a fragment thereof (e.g., at least a portion of the extracellular domain of PD1), and a second polypeptide of an intracellular signaling domain described herein (e.g., a CD28 signaling domain described herein and/or a CD3 zeta signaling domain described herein).

In one aspect, the present invention provides methods comprising administering a population of CAR-expressing cells, e.g., CART cells, e.g., a mixture of cells expressing different CARs, in combination with another agent, e.g., a PD-1 inhibitor, such as a PD-1 inhibitor described herein. In another aspect, the present invention provides methods comprising administering a population of cells wherein at least one cell in the population expresses a CAR having an anti-CD19 binding domain as described herein, and a second cell expressing another agent, e.g., an agent which enhances the activity or fitness of a CAR-expressing cell, in combination with another agent, e.g., a PD-1 inhibitor, such as a PD-1 inhibitor described herein.

### PD-1 Inhibitors

The immune system has the capability of recognizing and eliminating tumor cells; however, tumors can use multiple strategies to evade immunity. Blockade of immune checkpoints is an approach to activating or reactivating therapeutic antitumor immunity. PD-1 is an exemplary immune checkpoint molecule.

PD-1 is a CD28/CTLA-4 family member expressed, e.g., on activated CD4⁺ and CD8⁺ T cells, T_{regs}, and B cells. See, e.g., Agata et al. 1996 Int. Immunol 8:765-75. PD-1 is an inhibitory member of the CD28 family of receptors that also includes CD28, CTLA-4, ICOS, and BTLA. PD-1 negatively regulates effector T cell signaling and function. PD-1 is induced on tumor-infiltrating T cells, and can result in functional exhaustion or dysfunction (Keir et al. (2008) Annu. Rev. Immunol. 26:677-704; Pardoll et al. (2012) Nat Rev Cancer 12(4):252-64). PD-1 delivers a coinhibitory signal upon binding to either of its two ligands, Programmed Death-Ligand 1 (PD-L1) or Programmed Death-Ligand 2 (PD-L2). PD-L1 and PD-L2 have been shown to downregulate T cell activation upon binding to PD-1 (Freeman et a. 2000 J Exp Med 192:1027-34; Latchman et al. 2001 Nat Immunol 2:261-8; Carter et al. 2002 Eur J Immunol 32:634-43). PD-L1 is expressed on a number of cell types, including T cells, natural killer (NK) cells, macrophages, dendritic cells (DCs), B cells, epithelial cells, vascular endothelial cells, as well as many types of tumors. PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094), and high expression of PD-L1 on murine and human tumors has been linked to poor clinical outcomes in a variety of cancers (Keir et al. (2008) Annu. Rev. Immunol. 26:677-704; Pardoll et al. (2012) Nat Rev Cancer 12(4):252-64). PD-L2 is expressed on dendritic cells, macrophages, and some tumors. Blockade of the PD-1 pathway has been pre-clinically and clinically validated for cancer immunotherapy. Immune suppression can be reversed by inhibiting the local interaction of PD-1 with PD-L1. Both preclinical and clinical studies have demonstrated that anti-PD-1 blockade can restore activity of effector T cells and results in robust anti-tumor response. For example, blockade of PD-1 pathway can restore exhausted/dysfunctional effector T cell function (e.g., proliferation, IFN-γ secretion, or cytolytic function) and/or inhibit T_{reg} cell function (Keir et al. (2008) Annu. Rev. Immunol. 26:677-704; Pardoll et al. (2012) Nat Rev Cancer 12(4):252-64). Blockade of the PD-1 pathway can be affected with an antibody, an antigen binding fragment thereof, an immunoadhesin, a fusion protein, or oligopeptide of PD-1, PD-L1 and/or PD-L2.

### Antibody Molecules to PD-1

In one embodiment, the PD-1 inhibitor is an anti-PD-1 antibody molecule as described in US 2015/0210769, published on July 30, 2015, entitled “Antibody Molecules to PD-1 and Uses Thereof,” incorporated by reference in its entirety.

In some embodiments, the anti-PD-1 antibody molecule (e.g., an isolated or recombinant antibody molecule) has one or more of the following properties:

(i) binds to PD-1, e.g., human PD-1, with high affinity, e.g., with an affinity constant of at least about 10⁷ M⁻¹, typically about 10⁸ M⁻¹, and more typically, about 10⁹ M⁻¹ to 10¹⁰ M⁻¹ or stronger;

(ii) does not substantially bind to CD28, CTLA-4, ICOS or BTLA;

(iii) inhibits or reduces binding of PD-1 to a PD-1 ligand, e.g., PD-L1 or PD-L2, or both;

(iv) binds specifically to an epitope on PD-1, e.g., the same or similar epitope as the epitope recognized by murine monoclonal antibody BAP049 or a chimeric antibody BAP049, e.g., BAP049-chi or BAP049-chi-Y;

(v) shows the same or similar binding affinity or specificity, or both, as any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E;

(vi) shows the same or similar binding affinity or specificity, or both, as an antibody molecule (e.g., an heavy chain variable region and light chain variable region) described in Table 6;

(vii) shows the same or similar binding affinity or specificity, or both, as an antibody molecule (e.g., an heavy chain variable region and light chain variable region) having an amino acid sequence shown in Table 6;

(viii) shows the same or similar binding affinity or specificity, or both, as an antibody molecule (e.g., an heavy chain variable region and light chain variable region) encoded by the nucleotide sequence shown in Table 6;

(ix) inhibits, e.g., competitively inhibits, the binding of a second antibody molecule to PD-1, wherein the second antibody molecule is an antibody molecule described herein, e.g., an antibody molecule chosen from, e.g., any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E;

(x) binds the same or an overlapping epitope with a second antibody molecule to PD-1, wherein the second antibody molecule is an antibody molecule described herein, e.g., an antibody molecule chosen from, e.g., any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E;

(xi) competes for binding, and/or binds the same epitope, with a second antibody molecule to PD-1, wherein the second antibody molecule is an antibody molecule described herein, e.g., an antibody molecule chosen from, e.g., any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E;

(xii) has one or more biological properties of an antibody molecule described herein, e.g., an antibody molecule chosen from, e.g., any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E;

(xiii) has one or more pharmacokinetic properties of an antibody molecule described herein, e.g., an antibody molecule chosen from, e.g., any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E;

(xiv) inhibits one or more activities of PD-1, e.g., results in one or more of: an increase in tumor infiltrating lymphocytes, an increase in T-cell receptor mediated proliferation, or a decrease in immune evasion by cancerous cells;

(xv) binds human PD-1 and is cross-reactive with cynomolgus PD-1;

(xvi) binds to one or more residues within the C strand, CC′ loop, C′ strand, or FG loop of PD-1, or a combination two, three or all of the C strand, CC′ loop, C′ strand or FG loop of PD-1, e.g., wherein the binding is assayed using ELISA or Biacore; or

(xvii) has a VL region that contributes more to binding to PD-1 than a VH region.

In some embodiments, the antibody molecule binds to PD-1 with high affinity, e.g., with a K_{D} that is about the same, or at least about 10%, 20%, 30%, 40%, 50%, 60%, 70%, 80% or 90% higher or lower than the K_{D} of a murine or chimeric anti-PD-1 antibody molecule, e.g., a murine or chimeric anti-PD-1 antibody molecule described herein. In some embodiments, the K_{D} of the murine or chimeric anti-PD-1 antibody molecule is less than about 0.4, 0.3, 0.2, 0.1, or 0.05 nM, e.g., measured by a Biacore method. In some embodiments, the K_{D} of the murine or chimeric anti-PD-1 antibody molecule is less than about 0.2 nM, e.g., about 0.135 nM. In other embodiments, the K_{D} of the murine or chimeric anti PD-1 antibody molecule is less than about 10, 5, 3, 2, or 1 nM, e.g., measured by binding on cells expressing PD-1 (e.g., 300.19 cells). In some embodiments, the K_{D} of the murine or chimeric anti PD-1 antibody molecule is less than about 5 nM, e.g., about 4.60 nM (or about 0.69 μg/mL).

In some embodiments, the anti-PD-1 antibody molecule binds to PD-1 with a K_{off} slower than 1×10⁴, 5×10⁻⁵, or 1×10⁻⁵ S⁻¹, e.g., about 1.65×10⁻⁵ s⁻¹. In some embodiments, the anti-PD-1 antibody molecule binds to PD-1 with a Kₒₙ faster than 1×10⁴, 5×10⁴, 1×10⁵, or 5×10⁵ M⁻¹s⁻¹, e.g., about 1.23×10⁵ M⁻¹s⁻¹.

In some embodiments, the expression level of the antibody molecule is higher, e.g., at least about 0.5, 1, 2, 3, 4, 5, 6, 7, 8, 9 or 10-fold higher, than the expression level of a murine or chimeric antibody molecule, e.g., a murine or chimeric anti-PD-1 antibody molecule described herein. In some embodiments, the antibody molecule is expressed in CHO cells.

In some embodiments, the anti-PD-1 antibody molecule reduces one or more PD-1-associated activities with an IC₅₀ (concentration at 50% inhibition) that is about the same or lower, e.g., at least about 10%, 20%, 30%, 40%, 50%, 60%, 70%, 80% or 90% lower, than the

IC₅₀ of a murine or chimeric anti-PD-1 antibody molecule, e.g., a murine or chimeric anti-PD-1 antibody molecule described herein. In some embodiments, the IC₅₀ of the murine or chimeric anti-PD-1 antibody molecule is less than about 6, 5, 4, 3, 2, or 1 nM, e.g., measured by binding on cells expressing PD-1 (e.g., 300.19 cells). In some embodiments, the IC₅₀ of the murine or chimeric anti-PD-1 antibody molecule is less than about 4 nM, e.g., about 3.40 nM (or about 0.51 μg/mL). In some embodiments, the PD-1-associated activity reduced is the binding of PD-L1 and/or PD-L2 to PD-1. In some embodiments, the anti-PD-1 antibody molecule binds to peripheral blood mononucleated cells (PBMCs) activated by Staphylococcal enterotoxin B (SEB). In other embodiments, the anti-PD-1 antibody molecule increases the expression of IL-2 on whole blood activated by SEB. For example, the anti-PD-1 antibody increases the expression of IL-2 by at least about 2, 3, 4, or 5-fold, compared to the expression of IL-2 when an isotype control (e.g., IgG4) is used.

In some embodiments, the anti-PD-1 antibody molecule has improved stability, e.g., at least about 0.5, 1, 2, 3, 4, 5, 6, 7, 8, 9 or 10-fold more stable in vivo or in vitro, than a murine or chimeric anti-PD-1 antibody molecule, e.g., a murine or chimeric anti-PD-1 antibody molecule described herein.

In one embodiment, the anti-PD-1 antibody molecule is a humanized antibody molecule and has a risk score based on T cell epitope analysis of 300 to 700, 400 to 650, 450 to 600, or a risk score as described herein.

In another embodiment, the anti-PD-1 antibody molecule comprises at least one antigen-binding region, e.g., a variable region or an antigen-binding fragment thereof, from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule comprises at least one, two, three or four variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule comprises at least one or two heavy chain variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule comprises at least one or two light chain variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule includes a heavy chain constant region for an IgG4, e.g., a human IgG4. In one embodiment, the human IgG4 includes a substitution at position 228 according to EU numbering (e.g., a Ser to Pro substitution). In still another embodiment, the anti-PD-1 antibody molecule includes a heavy chain constant region for an IgG1, e.g., a human IgG1. In one embodiment, the human IgG1 includes a substitution at position 297 according to EU numbering (e.g., an Asn to Ala substitution). In one embodiment, the human IgG1 includes a substitution at position 265 according to EU numbering, a substitution at position 329 according to EU numbering, or both (e.g., an Asp to Ala substitution at position 265 and/or a Pro to Ala substitution at position 329). In one embodiment, the human IgG1 includes a substitution at position 234 according to EU numbering, a substitution at position 235 according to EU numbering, or both (e.g., a Leu to Ala substitution at position 234 and/or a Leu to Ala substitution at position 235). In one embodiment, the heavy chain constant region comprises an amino sequence set forth in Table 3, or a sequence substantially identical (e.g., at least 80%, 85%, 90%, 92%, 95%, 97%, 98%, 99% or higher identical) thereto.

In yet another embodiment, the anti-PD-1 antibody molecule includes a kappa light chain constant region, e.g., a human kappa light chain constant region. In one embodiment, the light chain constant region comprises an amino sequence set forth in Table 3 of US 2015/0210769A1, or a sequence substantially identical (e.g., at least 80%, 85%, 90%, 92%, 95%, 97%, 98%, 99% or higher identical) thereto.

In another embodiment, the anti-PD-1 antibody molecule includes a heavy chain constant region for an IgG4, e.g., a human IgG4, and a kappa light chain constant region, e.g., a human kappa light chain constant region, e.g., a heavy and light chain constant region comprising an amino sequence set forth in Table 3 of US 2015/0210769A1, or a sequence substantially identical (e.g., at least 80%, 85%, 90%, 92%, 95%, 97%, 98%, 99% or higher identical) thereto. In one embodiment, the human IgG4 includes a substitution at position 228 according to EU numbering (e.g., a Ser to Pro substitution). In yet another embodiment, the anti-PD-1 antibody molecule includes a heavy chain constant region for an IgG1, e.g., a human IgG1, and a kappa light chain constant region, e.g., a human kappa light chain constant region, e.g., a heavy and light chain constant region comprising an amino sequence set forth in Table 3 of US 2015/0210769A1, or a sequence substantially identical (e.g., at least 80%, 85%, 90%, 92%, 95%, 97%, 98%, 99% or higher identical) thereto. In one embodiment, the human IgG1 includes a substitution at position 297 according to EU numbering (e.g., an Asn to Ala substitution). In one embodiment, the human IgG1 includes a substitution at position 265 according to EU numbering, a substitution at position 329 according to EU numbering, or both (e.g., an Asp to Ala substitution at position 265 and/or a Pro to Ala substitution at position 329). In one embodiment, the human IgG1 includes a substitution at position 234 according to EU numbering, a substitution at position 235 according to EU numbering, or both (e.g., a Leu to Ala substitution at position 234 and/or a Leu to Ala substitution at position 235).

In another embodiment, the anti-PD-1 antibody molecule includes a heavy chain variable domain and a constant region, a light chain variable domain and a constant region, or both, comprising the amino acid sequence of BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences. The anti-PD-1 antibody molecule, optionally, comprises a leader sequence from a heavy chain, a light chain, or both, as showin in Table 4 of US 2015/0210769A1; or a sequence substantially identical thereto.

In yet another embodiment, the anti-PD-1 antibody molecule includes at least one, two, or three complementarity determining regions (CDRs) from a heavy chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences.

In yet another embodiment, the anti-PD-1 antibody molecule includes at least one, two, or three CDRs (or collectively all of the CDRs) from a heavy chain variable region comprising an amino acid sequence shown in Table 6, or encoded by a nucleotide sequence shown in Table 6. In one embodiment, one or more of the CDRs (or collectively all of the CDRs) have one, two, three, four, five, six or more changes, e.g., amino acid substitutions or deletions, relative to the amino acid sequence shown in Table 6, or encoded by a nucleotide sequence shown in Table 6.

In yet another embodiment, the anti-PD-1 antibody molecule includes at least one, two, or three CDRs from a light chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequence.

In yet another embodiment, the anti-PD-1 antibody molecule includes at least one, two, or three CDRs (or collectively all of the CDRs) from a light chain variable region comprising an amino acid sequence shown in Table 6, or encoded by a nucleotide sequence shown in Table 6. In one embodiment, one or more of the CDRs (or collectively all of the CDRs) have one, two, three, four, five, six or more changes, e.g., amino acid substitutions or deletions, relative to the amino acid sequence shown in Table 6, or encoded by a nucleotide sequence shown in Table 6. In certain embodiments, the anti-PD-1 antibody molecule includes a substitution in a light chain CDR, e.g., one or more substitutions in a CDR1, CDR2 and/or CDR3 of the light chain. In one embodiment, the anti-PD-1 antibody molecule includes a substitution in the light chain CDR3 at position 102 of the light variable region, e.g., a substitution of a cysteine to tyrosine, or a cysteine to serine residue, at position 102 of the light variable region according to Table 6 (e.g., SEQ ID NO: 152 or 162 for murine or chimeric, unmodified; or any of SEQ ID NOs: 168, 176, 180, 188, 192, 196, 200, 204, 208, or 212 for a modified sequence).

In another embodiment, the anti-PD-1 antibody molecule includes at least one, two, three, four, five or six CDRs (or collectively all of the CDRs) from a heavy and light chain variable region comprising an amino acid sequence shown in Table 6, or encoded by a nucleotide sequence shown in Table 6. In one embodiment, one or more of the CDRs (or collectively all of the CDRs) have one, two, three, four, five, six or more changes, e.g., amino acid substitutions or deletions, relative to the amino acid sequence shown in Table 6, or encoded by a nucleotide sequence shown in Table 6.

In one embodiment, the anti-PD-1 antibody molecule includes all six CDRs from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6, or closely related CDRs, e.g., CDRs which are identical or which have at least one amino acid alteration, but not more than two, three or four alterations (e.g., substitutions, deletions, or insertions, e.g., conservative substitutions). In one embodiment, the anti-PD-1 antibody molecule may include any CDR described herein. In certain embodiments, the anti-PD-1 antibody molecule includes a substitution in a light chain CDR, e.g., one or more substitutions in a CDR1, CDR2 and/or CDR3 of the light chain. In one embodiment, the anti-PD-1 antibody molecule includes a substitution in the light chain CDR3 at position 102 of the light variable region, e.g., a substitution of a cysteine to tyrosine, or a cysteine to serine residue, at position 102 of the light variable region according to Table 6 (e.g., SEQ ID NO: 152 or 162 for murine or chimeric, unmodified; or any of SEQ ID NOs: 168, 176, 180, 188, 192, 196, 200, 204, 208, or 212 for a modified sequence).

In another embodiment, the anti-PD-1 antibody molecule includes at least one, two, or three CDRs according to Kabat et al. (e.g., at least one, two, or three CDRs according to the Kabat definition as set out in Table 6) from a heavy chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 6.

In another embodiment, the anti-PD-1 antibody molecule includes at least one, two, or three CDRs according to Kabat et al. (e.g., at least one, two, or three CDRs according to the Kabat definition as set out in Table 6) from a light chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 6.

In yet another embodiment, the anti-PD-1 antibody molecule includes at least one, two, three, four, five, or six CDRs according to Kabat et al. (e.g., at least one, two, three, four, five, or six CDRs according to the Kabat definition as set out in Table 6) from the heavy and light chain variable regions of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 6.

In yet another embodiment, the anti-PD-1 antibody molecule includes all six CDRs according to Kabat et al. (e.g., all six CDRs according to the Kabat definition as set out in Table 6) from the heavy and light chain variable regions of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 6. In one embodiment, the anti-PD-1 antibody molecule may include any CDR described herein.

In another embodiment, the anti-PD-1 antibody molecule includes at least one, two, or three Chothia hypervariable loops (e.g., at least one, two, or three hypervariable loops according to the Chothia definition as set out in Table 6) from a heavy chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, or three hypervariable loops according to Chothia et al. shown in Table 6.

In another embodiment, the anti-PD-1 antibody molecule includes at least one, two, or three Chothia hypervariable loops (e.g., at least one, two, or three hypervariable loops according to the Chothia definition as set out in Table 6) of a light chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, or three hypervariable loops according to Chothia et al. shown in Table 6.

In yet another embodiment, the anti-PD-1 antibody molecule includes at least one, two, three, four, five, or six hypervariable loops (e.g., at least one, two, three, four, five, or six hypervariable loops according to the Chothia definition as set out in Table 6) from the heavy and light chain variable regions of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 6, or encoded by the nucleotide sequence in Table 6; or at least the amino acids from those hypervariable loops that contact PD-1; or which have at least one amino acid alteration, but not more than two, three or four alterations (e.g., substitutions, deletions, or insertions, e.g., conservative substitutions) relative to one, two, three, four, five or six hypervariable loops according to Chothia et al. shown in Table 6.

In one embodiment, the anti-PD-1 antibody molecule includes all six hypervariable loops (e.g., all six hypervariable loops according to the Chothia definition as set out in Table 6) of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E, or closely related hypervariable loops, e.g., hypervariable loops which are identical or which have at least one amino acid alteration, but not more than two, three or four alterations (e.g., substitutions, deletions, or insertions, e.g., conservative substitutions); or which have at least one amino acid alteration, but not more than two, three or four alterations (e.g., substitutions, deletions, or insertions, e.g., conservative substitutions) relative to all six hypervariable loops according to Chothia et al. shown in Table 6. In one embodiment, the anti-PD-1 antibody molecule may include any hypervariable loop described herein.

In still another embodiment, the anti-PD-1 antibody molecule includes at least one, two, or three hypervariable loops that have the same canonical structures as the corresponding hypervariable loop of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In certain embodiments, the anti-PD-1 antibody molecule includes a combination of CDRs or hypervariable loops defined according to the Kabat et al. and Chothia et al.

In one embodiment, the anti-PD-1 antibody molecule includes at least one, two or three CDRs or hypervariable loops from a heavy chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E, according to the Kabat and Chothia definition (e.g., at least one, two, or three CDRs or hypervariable loops according to the Kabat and Chothia definition as set out in Table 6); or encoded by the nucleotide sequence in Table 6; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs or hypervariable loops according to Kabat and/or Chothia shown in Table 6.

For example, the anti-PD-1 antibody molecule can include VH CDR1 according to Kabat et al. or VH hypervariable loop 1 according to Chothia et al., or a combination thereof, e.g., as shown in Table 6. In one embodiment, the combination of Kabat and Chothia CDR of VH CDR1 comprises the amino acid sequence GYTFTTYWMH (SEQ ID NO: 286), or an amino acid sequence substantially identical thereto (e.g., having at least one amino acid alteration, but not more than two, three or four alterations (e.g., substitutions, deletions, or insertions, e.g., conservative substitutions)). The anti-PD-1 antibody molecule can further include, e.g., VH CDRs 2-3 according to Kabat et al. and VL CDRs 1-3 according to Kabat et al., e.g., as shown in Table 6. Accordingly, in some embodiments, framework regions are defined based on a combination of CDRs defined according to Kabat et al. and hypervariable loops defined according to Chothia et al. For example, the anti-PD-1 antibody molecule can include VH FR1 defined based on VH hypervariable loop 1 according to Chothia et al. and VH FR2 defined based on VH CDRs 1-2 according to Kabat et al., e.g., as shown in Table 6. The anti-PD-1 antibody molecule can further include, e.g., VH FRs 3-4 defined based on VH CDRs 2-3 according to Kabat et al. and VL FRs 1-4 defined based on VL CDRs 1-3 according to Kabat et al.

The anti-PD-1 antibody molecule can contain any combination of CDRs or hypervariable loops according to the Kabat and Chothia definitions. In one embodiment, the anti-PD-1 antibody molecule includes at least one, two or three CDRs from a light chain variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E, according to the Kabat and Chothia definition (e.g., at least one, two, or three CDRs according to the Kabat and Chothia definition as set out in Table 6).

In an embodiment, e.g., an embodiment comprising a variable region, a CDR (e.g., Chothia CDR or Kabat CDR), or other sequence referred to herein, e.g., in Table 6, the antibody molecule is a monospecific antibody molecule, a bispecific antibody molecule, or is an antibody molecule that comprises an antigen binding fragment of an antibody, e.g., a half antibody or antigen binding fragment of a half antibody. In certain embodiments the antibody molecule is a bispecific antibody molecule having a first binding specificity for PD-1 and a second binding specificity for TIM-3, LAG-3, CEACAM (e.g., CEACAM-1 and/or CEACAM-5), PD-L1 or PD-L2.

In one embodiment, the anti-PD-1 antibody molecule includes:

(a) a heavy chain variable region (VH) comprising a VHCDR1 amino acid sequence of SEQ ID NO: 140, a VHCDR2 amino acid sequence of SEQ ID NO: 141, and a VHCDR3 amino acid sequence of SEQ ID NO: 139; and a light chain variable region (VL) comprising a VLCDR1 amino acid sequence of SEQ ID NO: 149, a VLCDR2 amino acid sequence of SEQ ID NO: 150, and a VLCDR3 amino acid sequence of SEQ ID NO: 167;

(b) a VH comprising a VHCDR1 amino acid sequence chosen from SEQ ID NO: 137; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166;

(c) a VH comprising a VHCDR1 amino acid sequence of SEQ ID NO: 286, a VHCDR2 amino acid sequence of SEQ ID NO: 141, and a VHCDR3 amino acid sequence of SEQ ID NO: 139; and a VL comprising a VLCDR1 amino acid sequence of SEQ ID NO: 149, a VLCDR2 amino acid sequence of SEQ ID NO: 150, and a VLCDR3 amino acid sequence of SEQ ID NO: 167; or

(d) a VH comprising a VHCDR1 amino acid sequence of SEQ ID NO: 286; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166.

In one embodiment, the anti-PD-1 antibody molecule comprises a VH comprising a VHCDR1 amino acid sequence of SEQ ID NO: 140, a VHCDR2 amino acid sequence of SEQ ID NO: 141, and a VHCDR3 amino acid sequence of SEQ ID NO: 139; and a VL comprising a VLCDR1 amino acid sequence of SEQ ID NO: 149, a VLCDR2 amino acid sequence of SEQ ID NO: 150, and a VLCDR3 amino acid sequence of SEQ ID NO: 167.

In one embodiment, the anti-PD-1 antibody molecule comprises a VH comprising a VHCDR1 amino acid sequence of SEQ ID NO: 137; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166.

In one embodiment, the anti-PD-1 antibody molecule comprises a VH comprising a VHCDR1 amino acid sequence of SEQ ID NO: 286, a VHCDR2 amino acid sequence of SEQ ID NO: 141, and a VHCDR3 amino acid sequence of SEQ ID NO: 139; and a VL comprising a VLCDR1 amino acid sequence of SEQ ID NO: 149, a VLCDR2 amino acid sequence of SEQ ID NO: 150, and a VLCDR3 amino acid sequence of SEQ ID NO: 167.

In one embodiment, the anti-PD-1 antibody molecule comprises a VH comprising a VHCDR1 amino acid sequence of SEQ ID NO: 286; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166.

In one embodiment, the antibody molecule is a humanized antibody molecule. In another embodiment, the antibody molecule is a monospecific antibody molecule. In yet another embodiment, the antibody molecule is a bispecific antibody molecule.

In one embodiment, the anti-PD-1 antibody molecule includes:

(i) a heavy chain variable region (VH) including a VHCDR1 amino acid sequence chosen from SEQ ID NO: 137, SEQ ID NO: 140 or SEQ ID NO: 286; a VHCDR2 amino acid sequence of SEQ ID NO: 138; and a VHCDR3 amino acid sequence of SEQ ID NO: 139; and

(ii) a light chain variable region (VL) including a VLCDR1 amino acid sequence of SEQ ID NO: 146, a VLCDR2 amino acid sequence of SEQ ID NO: 147, and a VLCDR3 amino acid sequence of SEQ ID NO: 166.

In another embodiment, the anti-PD-1 antibody molecule includes:

(i) a heavy chain variable region (VH) including a VHCDR1 amino acid sequence chosen from SEQ ID NO: 137, SEQ ID NO: 140 or SEQ ID NO: 286; a VHCDR2 amino acid sequence of SEQ ID NO: 141, and a VHCDR3 amino acid sequence of SEQ ID NO: 139; and

(ii) a light chain variable region (VL) including a VLCDR1 amino acid sequence of SEQ ID NO: 149, a VLCDR2 amino acid sequence of SEQ ID NO: 150, and a VLCDR3 amino acid sequence of SEQ ID NO: 167.

In one embodiment, the anti-PD-1 antibody molecule comprises the VHCDR1 amino acid sequence of SEQ ID NO: 137. In another embodiment, the anti-PD-1 antibody molecule comprises the VHCDR1 amino acid sequence of SEQ ID NO: 140. In yet another embodiment, the anti-PD-1 antibody molecule comprises the VHCDR1 amino acid sequence of SEQ ID NO: 286.

In one embodiment, the light or the heavy chain variable framework (e.g., the region encompassing at least FR1, FR2, FR3, and optionally FR4) of the anti-PD-1 antibody molecule can be chosen from: (a) a light or heavy chain variable framework including at least 80%, 85%, 87% 90%, 92%, 93%, 95%, 97%, 98%, or preferably 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

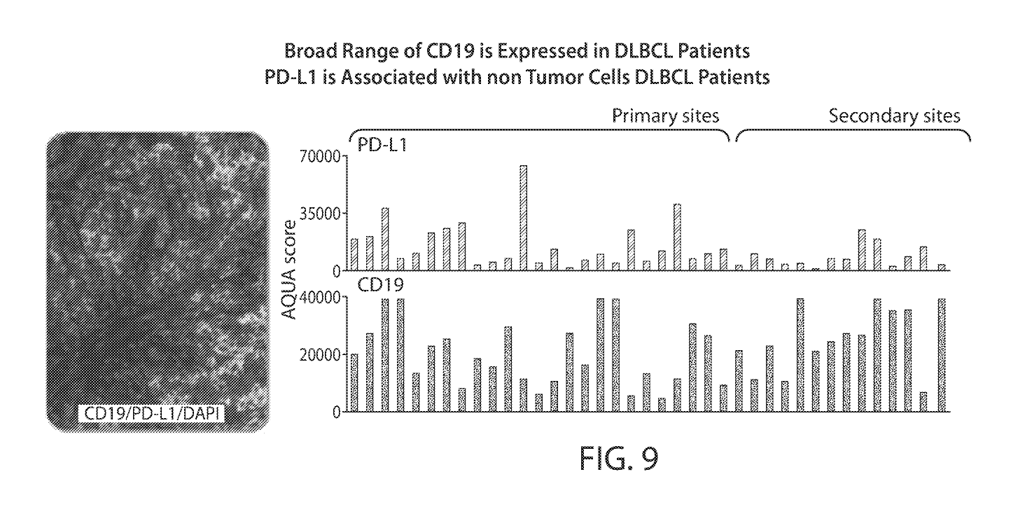

In certain embodiments, the anti-PD-1 antibody molecule comprises a heavy chain variable domain having at least one, two, three, four, five, six, seven, ten, fifteen, twenty or more changes, e.g., amino acid substitutions or deletions, from an amino acid sequence of BAP049-chi-HC, e.g., the amino acid sequence of the FR region in the entire variable region, e.g., shown in FIGS. 9A-9B of US 2015/0210769A1, or SEQ ID NO: 154, 156, 158 or 160. In one embodiment, the anti-PD-1 antibody molecule comprises a heavy chain variable domain having one or more of: E at position 1, V at position 5, A at position 9, V at position 11, K at position 12, K at position 13, E at position 16, L at position 18, R at position 19, I or V at position 20, G at position 24, I at position 37, A or S at position 40, T at position 41, S at position 42, R at position 43, M or L at position 48, V or F at position 68, T at position 69, I at position 70, S at position 71, A or R at position 72, K or N at position 74, T or K at position 76, S or N at position 77, L at position 79, L at position 81, E or Q at position 82, M at position 83, S or N at position 84, R at position 87, A at position 88, or T at position 91 of amino acid sequence of BAP049-chi-HC, e.g., the amino acid sequence of the FR in the entire variable region, e.g., shown in FIGS. 9A-9B of US 2015/0210769A1, or SEQ ID NO: 154, 156, 158 or 160.

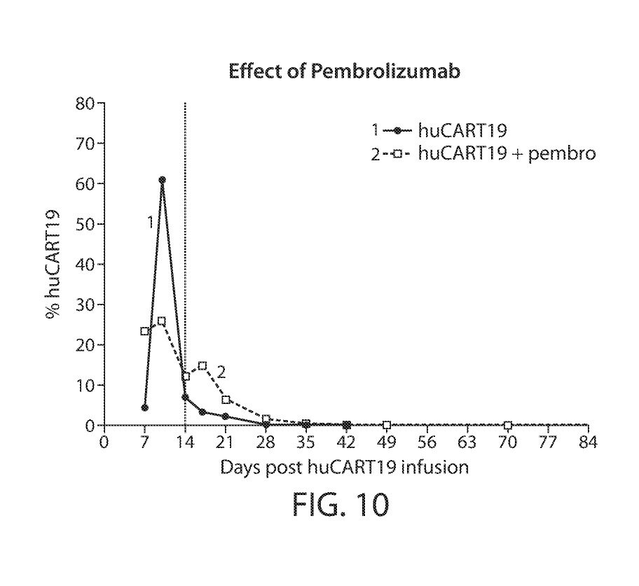

Alternatively, or in combination with the heavy chain substitutions of BAP049-chi-HC described herein, the anti-PD-1 antibody molecule comprises a light chain variable domain having at least one, two, three, four, five, six, seven, ten, fifteen, twenty or more amino acid changes, e.g., amino acid substitutions or deletions, from an amino acid sequence of BAP049-chi-LC, e.g., the amino acid sequence shown in FIGS. 10A-10B of US 2015/0210769A1, or SEQ ID NO: 162 or 164. In one embodiment, the anti-PD-1 antibody molecule comprises a heavy chain variable domain having one or more of: E at position 1, V at position 2, Q at position 3, L at position 4, T at position 7, D or L or A at position 9, F or T at position 10, Q at position 11, S or P at position 12, L or A at position 13, S at position 14, P or L or V at position 15, K at position 16, Q or D at position 17, R at position 18, A at position 19, S at position 20, I or L at position 21, T at position 22, L at position 43, K at position 48, A or S at position 49, R or Q at position 51, Y at position 55, I at position 64, S or P at position 66, S at position 69, Y at position 73, G at position 74, E at position 76, F at position 79, N at position 82, N at position 83, L or I at position 84, E at position 85, S or P at position 86, D at position 87, A or F or I at position 89, T or Y at position 91, F at position 93, or Y at position 102 of the amino acid sequence of BAP049-chi-LC, e.g., the amino acid sequence shown in FIGS. 10A-10B of US 2015/0210769A1, or SEQ ID NO: 162 or 164.

In other embodiments, the anti-PD-1 antibody molecule includes one, two, three, or four heavy chain framework regions (e.g., a VHFW amino acid sequence shown in Table 2 of US 2015/0210769A1, or encoded by the nucleotide sequence shown in Table 2 of US 2015/0210769A1), or a sequence substantially identical thereto.

In yet other embodiments, the anti-PD-1 antibody molecule includes one, two, three, or four light chain framework regions (e.g., a VLFW amino acid sequence shown in Table 2 of US 2015/0210769A1, or encoded by the nucleotide sequence shown in Table 2 of US 2015/0210769A1), or a sequence substantially identical thereto.

In other embodiments, the anti-PD-1 antibody molecule includes one, two, three, or four heavy chain framework regions (e.g., a VHFW amino acid sequence shown in Table 2 of US 2015/0210769A1, or encoded by the nucleotide sequence shown in Table 2 of US 2015/0210769A1), or a sequence substantially identical thereto; and one, two, three, or four light chain framework regions (e.g., a VLFW amino acid equence shown in Table 2 of US 2015/0210769A1, or encoded by the nucleotide sequence shown in Table 2 of US 2015/0210769A1), or a sequence substantially identical thereto.

In some embodiments, the anti-PD-1 antibody molecule comprises the heavy chain framework region 1 (VHFW1) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum15, BAP049-hum16, BAP049-Clone-A, BAP049-Clone-B, BAP049-Clone-C, BAP049-Clone-D, or BAP049-Clone-E (e.g., SEQ ID NO: 147 of US 2015/0210769A1). In some embodiments, the antibody molecule comprises the heavy chain framework region 1 (VHFW1) of BAP049-hum14 or BAP049-hum15 (e.g., SEQ ID NO: 151 of US 2015/0210769A1).

In some embodiments, the anti-PD-1 antibody molecule comprises the heavy chain framework region 2 (VHFW2) of BAP049-hum01, BAP049-hum02, BAP049-hum05, BAP049-hum06, BAP049-hum07, BAP049-hum09, BAP049-hum11, BAP049-hum12, BAP049-hum13, BAP049-Clone-A, BAP049-Clone-B, BAP049-Clone-C, or BAP049-Clone-E (e.g., SEQ ID NO:

153 of US 2015/0210769A1). In some embodiments, the antibody molecule comprises the heavy chain framework region 2 (VHFW2) of BAP049-hum03, BAP049-hum04, BAP049-hum08, BAP049-hum10, BAP049-hum14, BAP049-hum15, or BAP049-Clone-D (e.g., SEQ ID NO: 157 of US 2015/0210769A1). In some embodiments, the antibody molecule comprises the heavy chain framework region 2 (VHFW2) of BAP049-hum16 (e.g., SEQ ID NO: 160 of US 2015/0210769A1).

In some embodiments, the anti-PD-1 antibody molecule comprises the heavy chain framework region 3 (VHFW3) of BAP049-hum01, BAP049-hum02, BAP049-hum05, BAP049-hum06, BAP049-hum07, BAP049-hum09, BAP049-hum11, BAP049-hum12, BAP049-hum13, BAP049-Clone-A, BAP049-Clone-B, BAP049-Clone-C, or BAP049-Clone-E (e.g., SEQ ID NO: 162 of US 2015/0210769A1). In some embodiments, the antibody molecule comprises the heavy chain framework region 3 (VHFW3) of BAP049-hum03, BAP049-hum04, BAP049-hum08, BAP049-hum10, BAP049-hum14, BAP049-hum15, BAP049-hum16, or BAP049-Clone-D (e.g., SEQ ID NO: 166 of US 2015/0210769A1).

In some embodiments, the anti-PD-1 antibody molecule comprises the heavy chain framework region 4 (VHFW4) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E (e.g., SEQ ID NO: 169 of US 2015/0210769A1).

In some embodiments, the anti-PD-1 antibody molecule comprises the light chain framework region 1 (VLFW1) of BAP049-hum08, BAP049-hum09, BAP049-hum15, BAP049-hum16, or BAP049-Clone-C (e.g., SEQ ID NO: 174 of US 2015/0210769A1). In some embodiments, the antibody molecule comprises the light chain framework region 1 (VLFW1) of BAP049-hum01, BAP049-hum04, BAP049-hum05, BAP049-hum07, BAP049-hum10, BAP049-hum11, BAP049-hum14, BAP049-Clone-A, BAP049-Clone-B, BAP049-Clone-D, or BAP049-Clone-E (e.g., SEQ ID NO: 177 of US 2015/0210769A1). In some embodiments, the antibody molecule comprises the light chain framework region 1 (VLFW1) of BAP049-hum06 (e.g., SEQ ID NO: 181 of US 2015/0210769A1). In some embodiments, the antibody molecule comprises the light chain framework region 1 (VLFW1) of BAP049-hum13 (e.g., SEQ ID NO: 183 of US 2015/0210769A1). In some embodiments, the antibody molecule comprises the light chain framework region 1 (VLFW1) of BAP049-hum02, BAP049-hum03, or BAP049-hum12 (e.g., SEQ ID NO: 185 of US 2015/0210769A1).

In some embodiments, the anti-PD-1 antibody molecule comprises the light chain framework region 2 (VLFW2) of BAP049-hum01, BAP049-hum02, BAP049-hum03, BAP049-hum06, BAP049-hum08, BAP049-hum09, BAP049-hum10, BAP049-hum11, BAP049-hum14, BAP049-hum15, BAP049-hum16, BAP049-Clone-A, BAP049-Clone-B, BAP049-Clone-D, or BAP049-Clone-E (e.g., SEQ ID NO: 187 of US 2015/0210769A1). In some embodiments, the antibody molecule comprises the light chain framework region 2 (VLFW2) of BAP049-hum04, BAP049-hum05, BAP049-hum07, BAP049-hum13, or BAP049-Clone-C (e.g., SEQ ID NO: 191 of US 2015/0210769A1). In some embodiments, the antibody molecule comprises the light chain framework region 2 (VLFW2) of BAP049-hum12 (e.g., SEQ ID NO: 194 of US 2015/0210769A1).

In some embodiments, the anti-PD-1 antibody molecule comprises the light chain framework region 3 (VLFW3) of BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-C, BAP049-Clone-D, or BAP049-Clone-E (e.g., SEQ ID NO: 196 of US 2015/0210769A1). In some embodiments, the antibody molecule comprises the light chain framework region 3 (VLFW3) of BAP049-hum02 or BAP049-hum03 (e.g., SEQ ID NO: 200 of US 2015/0210769A1). In some embodiments, the antibody molecule comprises the light chain framework region 3 (VLFW3) of BAP049-hum01 or BAP049-Clone-A (e.g., SEQ ID NO: 202 of US 2015/0210769A1). In some embodiments, the antibody molecule comprises the light chain framework region 3 (VLFW3) of BAP049-hum04, BAP049-hum05, or BAP049-Clone-B (e.g., SEQ ID NO: 205 of US 2015/0210769A1).

In some embodiments, the anti-PD-1 antibody molecule comprises the light chain framework region 4 (VLFW4) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E (e.g., SEQ ID NO: 208 of US 2015/0210769A1).

In some embodiments, the anti-PD-1 antibody molecule comprises the heavy chain framework regions 1-3 of BAP049-hum01, BAP049-hum02, BAP049-hum05, BAP049-hum06, BAP-hum07, BAP049-hum09, BAP049-hum11, BAP049-hum12, BAP049-hum13, BAP049-Clone-A, BAP049-Clone-B, BAP049-Clone-C, or BAP049-Clone-E (e.g., SEQ ID NO: 147 (VHFW1), SEQ ID NO: 153 (VHFW2), and SEQ ID NO: 162 (VHFW3) of US 2015/0210769A1). In some embodiments, the antibody molecule comprises the heavy chain framework regions 1-3 of BAP049-hum03, BAP049-hum04, BAP049-hum08, BAP049-hum10, or BAP049-Clone-D (e.g., SEQ ID NO: 147 (VHFW1), SEQ ID NO: 157 (VHFW2), and SEQ ID NO: 166 (VHFW3) of US 2015/0210769A1). In some embodiments, the antibody molecule comprises the heavy chain framework regions 1-3 of BAP049-hum14 or BAP049-hum15 (e.g., SEQ ID NO: 151 (VHFW1), SEQ ID NO: 157 (VHFW2), and SEQ ID NO: 166 (VHFW3) of US 2015/0210769A1). In some embodiments, the antibody molecule comprises the heavy chain framework regions 1-3 of BAP049-hum16 (e.g., SEQ ID NO: 147 (VHFW1), SEQ ID NO: 160 (VHFW2), and SEQ ID NO: 166 (VHFW3) of US 2015/0210769A1). In some embodiments, the antibody molecule further comprises the heavy chain framework region 4 (VHFW4) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E (e.g., SEQ ID NO: 169 of US 2015/0210769A1).

In some embodiments, the anti-PD-1 antibody molecule comprises the light chain framework regions 1-3 of BAP049-hum01 or BAP049-Clone-A (e.g., SEQ ID NO: 177 (VLFW1), SEQ ID NO: 187 (VLFW2), and SEQ ID NO: 202 (VLFW3) of US 2015/0210769A1). In some embodiments, the antibody molecule comprises the light chain framework regions 1-3 of BAP049-hum02 or BAP049-hum03 (e.g., SEQ ID NO: 185 (VLFW1), SEQ ID NO: 187 (VLFW2), and SEQ ID NO: 200 (VLFW3) of US 2015/0210769A1). In some embodiments, the antibody molecule comprises the light chain framework regions 1-3 of BAP049-hum04, BAP049-hum05, or BAP049-Clone-B (e.g., SEQ ID NO: 177 (VLFW1), SEQ ID NO: 191 (VLFW2), and SEQ ID NO: 205 (VLFW3) of US 2015/0210769A1). In some embodiments, the antibody molecule comprises the light chain framework regions 1-3 of BAP049-hum06 (e.g., SEQ ID NO: 181 (VLFW1), SEQ ID NO: 187 (VLFW2), and SEQ ID NO: 196 (VLFW3) of US 2015/0210769A1). In some embodiments, the antibody molecule comprises the light chain framework regions 1-3 of BAP049-hum07 (e.g., SEQ ID NO: 177 (VLFW1), SEQ ID NO: 191 (VLFW2), and SEQ ID NO: 196 (VLFW3) of US 2015/0210769A1). In some embodiments, the antibody molecule comprises the light chain framework regions 1-3 of BAP049-hum08, BAP049-hum09, BAP049-hum15, BAP049-hum16, or BAP049-Clone-C (e.g., SEQ ID NO: 174 (VLFW1), SEQ ID NO: 187 (VLFW2), and SEQ ID NO: 196 (VLFW3) of US 2015/0210769A1). In some embodiments, the antibody molecule comprises the light chain framework regions 1-3 of BAP049-hum10, BAP049-huml1, BAP049-hum14, BAP049-Clone-D, or BAP049-Clone-E (e.g., SEQ ID NO: 177 (VLFW1), SEQ ID NO: 187 (VLFW2), and SEQ ID NO: 196 (VLFW3) of US 2015/0210769A1). In some embodiments, the antibody molecule comprises the light chain framework regions 1-3 of BAP049-hum12 (e.g., SEQ ID NO: 185 (VLFW1), SEQ ID NO: 194 (VLFW2), and SEQ ID NO: 196 (VLFW3) of US 2015/0210769A1). In some embodiments, the antibody molecule comprises the light chain framework regions 1-3 of BAP049-hum13 (e.g., SEQ ID NO: 183 (VLFW1), SEQ ID NO: 191 (VLFW2), and SEQ ID NO: 196 (VLFW3) of US 2015/0210769A1). In some embodiments, the antibody molecule further comprises the light chain framework region 4 (VLFW4) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E (e.g., SEQ ID NO: 208 of US 2015/0210769A1).

In some embodiments, the anti-PD-1 antibody molecule comprises the heavy chain framework regions 1-3 of BAP049-hum01 or BAP049-Clone-A (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum01 or BAP049-Clone-A (e.g., SEQ ID NO: 177 (VLFW1), SEQ ID NO: 187 (VLFW2), and SEQ ID NO: 202 (VLFW3) of US 2015/0210769A1).

In some embodiments, the anti-PD-1 antibody molecule comprises the heavy chain framework regions 1-3 of BAP049-hum02 (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum02 (e.g., SEQ ID NO: 185 (VLFW1), SEQ ID NO: 187 (VLFW2), and SEQ ID NO: 200 (VLFW3) of US 2015/0210769A1).

In some embodiments, the anti-PD-1 antibody molecule comprises the heavy chain framework regions 1-3 of BAP049-hum03 (e.g., SEQ ID NO: 147 (VHFW1), SEQ ID NO: 157 (VHFW2), and SEQ ID NO: 166 (VHFW3) of US 2015/0210769A1) and the light chain framework regions 1-3 of BAP049-hum03 (e.g., SEQ ID NO: 185 (VLFW1), SEQ ID NO: 187 (VLFW2), and SEQ ID NO: 200 (VLFW3) of US 2015/0210769A1).

In some embodiments, the anti-PD-1 antibody molecule comprises the heavy chain framework regions 1-3 of BAP049-hum04 (e.g., SEQ ID NO: 147 (VHFW1), SEQ ID NO: 157 (VHFW2), and SEQ ID NO: 166 (VHFW3) of US 2015/0210769A1) and the light chain framework regions 1-3 of BAP049-hum04 (e.g., SEQ ID NO: 177 (VLFW1), SEQ ID NO: 191 (VLFW2), and SEQ ID NO: 205 (VLFW3) of US 2015/0210769A1).

In some embodiments, the anti-PD-1 antibody molecule comprises the heavy chain framework regions 1-3 of BAP049-hum05 or BAP049-Clone-B (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum05 or BAP049-Clone-B (e.g., SEQ ID NO: 177 (VLFW1), SEQ ID NO: 191 (VLFW2), and SEQ ID NO: 205 (VLFW3) of US 2015/0210769A1).

In some embodiments, the anti-PD-1 antibody molecule comprises the heavy chain framework regions 1-3 of BAP049-hum06 (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum06 (e.g., SEQ ID NO: 181 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum07 (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum07 (e.g., SEQ ID NO: 177 (VLFW1), SEQ ID NO: 191 (VLFW2), and SEQ ID NO: 196 (VLFW3) of US 2015/0210769A1).

In some embodiments, the anti-PD-1 antibody molecule comprises the heavy chain framework regions 1-3 of BAP049-hum08 (e.g., SEQ ID NO: 147 (VHFW1), SEQ ID NO: 157 (VHFW2), and SEQ ID NO: 166 (VHFW3) of US 2015/0210769A1) and the light chain framework regions 1-3 of BAP049-hum08 (e.g., SEQ ID NO: 174 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum09 or BAP049-Clone-C (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum09 or BAP049-Clone-C (e.g., SEQ ID NO: 174 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum10 or BAP049-Clone-D (e.g., SEQ ID NO: 147 (VHFW1), SEQ ID NO: 157 (VHFW2), and SEQ ID NO: 166 (VHFW3) of US 2015/0210769A1) and the light chain framework regions 1-3 of BAP049-hum10 or BAP049-Clone-D (e.g., SEQ ID NO: 177 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum11 or BAP049-Clone-E (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum11 or BAP049-Clone-E (e.g., SEQ ID NO: 177 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum12 (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum12 (e.g., SEQ ID NO: 185 (VLFW1), SEQ ID NO: 194 (VLFW2), and SEQ ID NO: 196 (VLFW3) of US 2015/0210769A1).

In some embodiments, the anti-PD-1 antibody molecule comprises the heavy chain framework regions 1-3 of BAP049-hum13 (e.g., SEQ ID NO: 147 (VHFW1), SEQ ID NO: 153 (VHFW2), and SEQ ID NO: 162 (VHFW3) of US 2015/0210769A1) and the light chain framework regions 1-3 of BAP049-hum13 (e.g., SEQ ID NO: 183 (VLFW1), SEQ ID NO: 191 (VLFW2), and SEQ ID NO: 196 (VLFW3) of US 2015/0210769A1).

In some embodiments, the anti-PD-1 antibody molecule comprises the heavy chain framework regions 1-3 of BAP049-hum14 (e.g., SEQ ID NO: 151 (VHFW1), SEQ ID NO: 157 (VHFW2), and SEQ ID NO: 166 (VHFW3) of US 2015/0210769A1) and the light chain framework regions 1-3 of BAP049-hum14 (e.g., SEQ ID NO: 177 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum15 (e.g., SEQ ID NO: 151 (VHFW1), SEQ ID NO: 157 (VHFW2), and SEQ ID NO: 166 (VHFW3) of US 2015/0210769A1) and the light chain framework regions 1-3 of BAP049-hum15 (e.g., SEQ ID NO: 174 (VLFW1), SEQ ID NO: 187 (VLFW2), and SEQ ID NO: 196 (VLFW3) of US 2015/0210769A1).

In some embodiments, the anti-PD-1 antibody molecule comprises the heavy chain framework regions 1-3 of BAP049-hum16 (e.g., SEQ ID NO: 147 (VHFW1), SEQ ID NO: 160 (VHFW2), and SEQ ID NO: 166 (VHFW3) of US 2015/0210769A1) and the light chain framework regions 1-3 of BAP049-hum16 (e.g., SEQ ID NO: 174 (VLFW1), SEQ ID NO: 187 (VLFW2), and SEQ ID NO: 196 (VLFW3) of US 2015/0210769A1).

In some embodiments, the anti-PD-1 antibody molecule further comprises the heavy chain framework region 4 (VHFW4) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E (e.g., SEQ ID NO: 169 of US 2015/0210769A1) and the light chain framework region 4 (VLFW4) of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E (e.g., SEQ ID NO: 208 of US 2015/0210769A1).

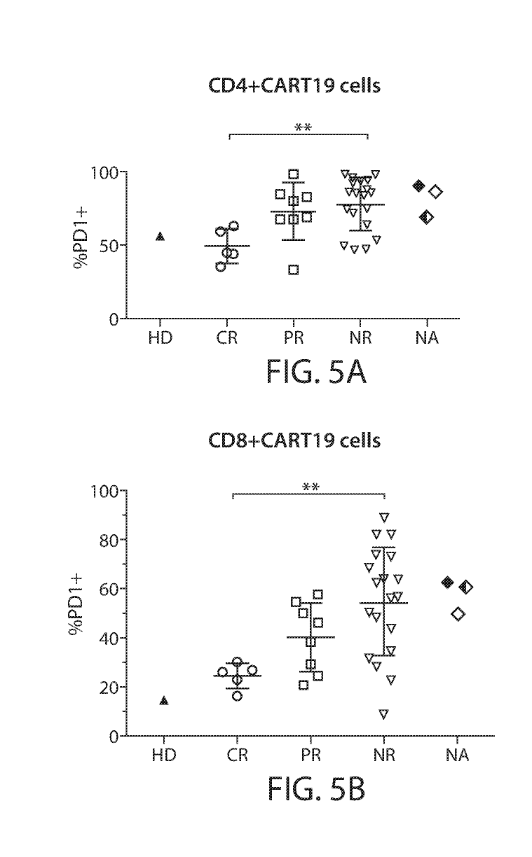

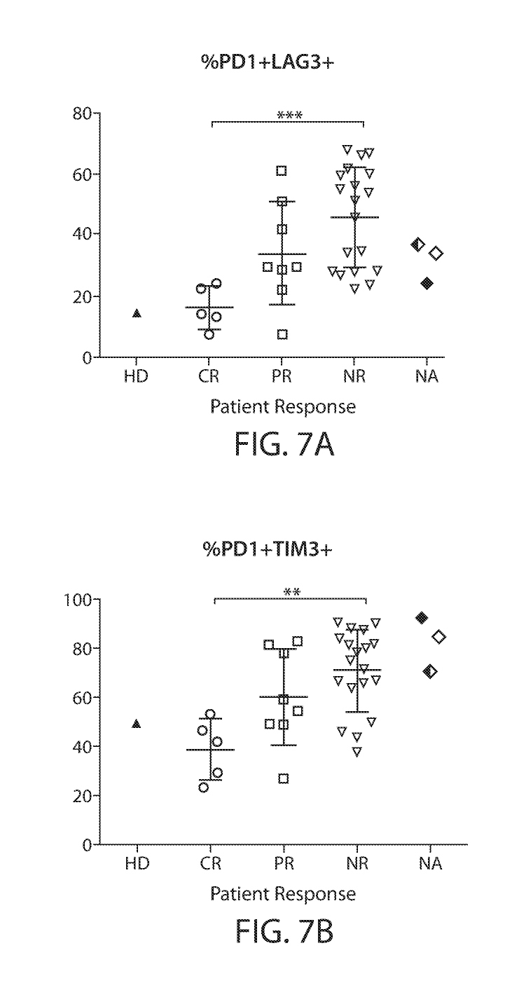

In some embodiments, the anti-PD-1 antibody molecule comprises a heavy chain framework region having a combination of framework regions FW1, FW2 and FW3 as show in FIG. 5 or 7 of US 2015/0210769A1. In other embodiment, the antibody molecule comprises a light chain framework region having a combination of framework regions FW1, FW2 and FW3 as show in FIG. 5 or 7 of US 2015/0210769A1. In yet other embodiments, the antibody molecule comprises a heavy chain framework region having a combination of framework regions FW1, FW2 and FW3 as show in FIG. 5 or 7 of US 2015/0210769A1, and a light chain framework region having a combination of framework regions FW1, FW2 and FW3 as showin in FIG. 5 or 7 of US 2015/0210769A1.

In one embodiment, the heavy or light chain variable domain, or both, of the anti-PD-1 antibody molecule includes an amino acid sequence, which is substantially identical to an amino acid disclosed herein, e.g., at least 80%, 85%, 90%, 92%, 95%, 97%, 98%, 99% or higher identical to a variable region of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-Clone-A, BAP049-Clone-B, BAP049-Clone-C, BAP049-Clone-D, or BAP049-Clone-E; or as described in Table 6, or encoded by the nucleotide sequence in Table 6; or which differs at least 1 or 5 residues, but less than 40, 30, 20, or 10 residues, from a variable region of an antibody described herein.

In one embodiment, the heavy or light chain variable region, or both, of the anti-PD-1 antibody molecule includes an amino acid sequence encoded by a nucleic acid sequence described herein or a nucleic acid that hybridizes to a nucleic acid sequence described herein (e.g., a nucleic acid sequence as shown in Tables 1 and 2 of US 2015/0210769A1, or Table 6 herein) or its complement, e.g., under low stringency, medium stringency, or high stringency, or other hybridization condition described herein.

In another embodiment, the anti-PD-1 antibody molecule comprises at least one, two, three, or four antigen-binding regions, e.g., variable regions, having an amino acid sequence as set forth in Table 6, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 6. In another embodiment, the anti-PD-1 antibody molecule includes a VH and/or VL domain encoded by a nucleic acid having a nucleotide sequence as set forth in Table 6, or a sequence substantially identical thereto (e.g., a sequence at least about 85%, 90%, 95%, 99% or more identical thereto, or which differs by no more than 3, 6, 15, 30, or 45 nucleotides from the sequences shown in Table 6.

In yet another embodiment, the anti-PD-1 antibody molecule comprises at least one, two, or three CDRs from a heavy chain variable region having an amino acid sequence as set forth in Table 6, or a sequence substantially homologous thereto (e.g., a sequence at least about 85%, 90%, 95%, 99% or more identical thereto, and/or having one, two, three or more substitutions, insertions or deletions, e.g., conserved substitutions). In yet another embodiment, the anti-PD-1 antibody molecule comprises at least one, two, or three CDRs from a light chain variable region having an amino acid sequence as set forth in Table 6, or a sequence substantially homologous thereto (e.g., a sequence at least about 85%, 90%, 95%, 99% or more identical thereto, and/or having one, two, three or more substitutions, insertions or deletions, e.g., conserved substitutions). In yet another embodiment, the anti-PD-1 antibody molecule comprises at least one, two, three, four, five or six CDRs from heavy and light chain variable regions having an amino acid sequence as set forth in Table 6), or a sequence substantially homologous thereto (e.g., a sequence at least about 85%, 90%, 95%, 99% or more identical thereto, and/or having one, two, three or more substitutions, insertions or deletions, e.g., conserved substitutions).

In one embodiment, the anti-PD-1 antibody molecule comprises at least one, two, or three CDRs and/or hypervariable loops from a heavy chain variable region having an amino acid sequence of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E, as summarized in Table 6, or a sequence substantially identical thereto (e.g., a sequence at least about 85%, 90%, 95%, 99% or more identical thereto, and/or having one, two, three or more substitutions, insertions or deletions, e.g., conserved substitutions). In another embodiment, the anti-PD-1 antibody molecule comprises at least one, two, or three CDRs and/or hypervariable loops from a light chain variable region having an amino acid sequence of an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E, as summarized in Table 6, or a sequence substantially identical thereto (e.g., a sequence at least about 85%, 90%, 95%, 99% or more identical thereto, and/or having one, two, three or more substitutions, insertions or deletions, e.g., conserved substitutions). In one embodiment, the anti-PD-1 antibody molecule comprises all six CDRs and/or hypervariable loops described herein, e.g., described in Table 6.

In one embodiment, the anti-PD-1 antibody molecule has a variable region that is identical in sequence, or which differs by 1, 2, 3, or 4 amino acids from a variable region described herein (e.g., an FR region disclosed herein).

In one embodiment, the anti-PD-1 antibody molecule is a full antibody or fragment thereof (e.g., a Fab, F(ab′)₂, Fv, or a single chain Fv fragment (scFv)). In certain embodiments, the anti-PD-1 antibody molecule is a monoclonal antibody or an antibody with single specificity. The anti-PD-1 antibody molecule can also be a humanized, chimeric, camelid, shark, or an in vitro-generated antibody molecule. In one embodiment, the anti-PD-1 antibody molecule thereof is a humanized antibody molecule. The heavy and light chains of the anti-PD-1 antibody molecule can be full-length (e.g., an antibody can include at least one, and preferably two, complete heavy chains, and at least one, and preferably two, complete light chains) or can include an antigen-binding fragment (e.g., a Fab, F(ab′)₂, Fv, a single chain Fv fragment, a single domain antibody, a diabody (dAb), a bivalent antibody, or bispecific antibody or fragment thereof, a single domain variant thereof, or a camelid antibody).

In yet other embodiments, the anti-PD-1 antibody molecule has a heavy chain constant region (Fc) chosen from, e.g., the heavy chain constant regions of IgG1, IgG2, IgG3, IgG4, IgM, IgA1, IgA2, IgD, and IgE; particularly, chosen from, e.g., the heavy chain constant regions of IgG1, IgG2, IgG3, and IgG4, more particularly, the heavy chain constant region of IgG1 or IgG2 (e.g., human IgG1, IgG2 or IgG4). In one embodiment, the heavy chain constant region is human

IgG1. In another embodiment, the anti-PD-1 antibody molecule has a light chain constant region chosen from, e.g., the light chain constant regions of kappa or lambda, preferably kappa (e.g., human kappa). In one embodiment, the constant region is altered, e.g., mutated, to modify the properties of the anti-PD-1 antibody molecule (e.g., to increase or decrease one or more of: Fc receptor binding, antibody glycosylation, the number of cysteine residues, effector cell function, or complement function). For example, the constant region is mutated at positions 296 (M to Y), 298 (S to T), 300 (T to E), 477 (H to K) and 478 (N to F) to alter Fc receptor binding (e.g., the mutated positions correspond to positions 132 (M to Y), 134 (S to T), 136 (T to E), 313 (H to K) and 314 (N to F) of SEQ ID NOs: 212 or 214; or positions 135 (M to Y), 137 (S to T), 139 (T to E), 316 (H to K) and 317 (N to F) of SEQ ID NOs: 215, 216, 217 or 218). In another embodiment, the heavy chain constant region of an IgG4, e.g., a human IgG4, is mutated at position 228 according to EU numbering (e.g., S to P), e.g., as shown in Table 3 of US 2015/0210769A1. In certain embodiments, the anti-PD-1 antibody molecules comprises a human IgG4 mutated at position 228 according to EU numbering (e.g., S to P), e.g., as shown in Table 3 of US 2015/0210769A1; and a kappa light chain constant region, e.g., as shown in Table 3 of US 2015/0210769A1. In still another embodiment, the heavy chain constant region of an IgG1, e.g., a human IgG1, is mutated at one or more of position 297 according to EU numbering (e.g., N to A), position 265 according to EU numbering (e.g., D to A), position 329 according to EU numbering (e.g., P to A), position 234 according to EU numbering (e.g., L to A), or position 235 according to EU numbering (e.g., L to A), e.g., as shown in Table 3 of US 2015/0210769A1.

In certain embodiments, the anti-PD-1 antibody molecules comprises a human IgG1 mutated at one or more of the aforesaid positions, e.g., as shown in Table 3 of US 2015/0210769A1; and a kappa light chain constant region, e.g., as shown in Table 3 of US 2015/0210769A1.

In one embodiment, the anti-PD-1 antibody molecule is isolated or recombinant.

In one embodiment, the anti-PD-1 antibody molecule is a humanized antibody molecule.

In one embodiment, the anti-PD-1 antibody molecule has a risk score based on T cell epitope analysis of less than 700, 600, 500, 400 or less.

In one embodiment, the anti-PD-1 antibody molecule is a humanized antibody molecule and has a risk score based on T cell epitope analysis of 300 to 700, 400 to 650, 450 to 600, or a risk score as described herein.

In one embodiment, the anti-PD-1 antibody molecule includes:

(a) a heavy chain variable region (VH) comprising a VHCDR1 amino acid sequence of SEQ ID NO: 140, a VHCDR2 amino acid sequence of SEQ ID NO: 141, and a VHCDR3 amino acid sequence of SEQ ID NO: 139; and a light chain variable region (VL) comprising a VLCDR1 amino acid sequence of SEQ ID NO: 149, a VLCDR2 amino acid sequence of SEQ ID NO: 150, and a VLCDR3 amino acid sequence of SEQ ID NO: 167;

(b) a VH comprising a VHCDR1 amino acid sequence chosen from SEQ ID NO: 137; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166;

(c) a VH comprising a VHCDR1 amino acid sequence of SEQ ID NO: 286, a VHCDR2 amino acid sequence of SEQ ID NO: 141, and a VHCDR3 amino acid sequence of SEQ ID NO: 139; and a VL comprising a VLCDR1 amino acid sequence of SEQ ID NO: 149, a VLCDR2 amino acid sequence of SEQ ID NO: 150, and a VLCDR3 amino acid sequence of SEQ ID NO: 167; or

(d) a VH comprising a VHCDR1 amino acid sequence of SEQ ID NO: 286; a VHCDR2 amino acid sequence of SEQ ID NO: 138; and a VHCDR3 amino acid sequence of SEQ ID NO: 139; and a VL comprising a VLCDR1 amino acid sequence of SEQ ID NO: 146, a VLCDR2 amino acid sequence of SEQ ID NO: 147, and a VLCDR3 amino acid sequence of SEQ ID NO: 166.

In certain embodiments, the anti-PD-1 antibody molecule comprises:

(i) a heavy chain variable region (VH) comprising a VHCDR1 amino acid sequence chosen from SEQ ID NO: 137, SEQ ID NO: 140 or SEQ ID NO: 286; a VHCDR2 amino acid sequence of SEQ ID NO: 138; and a VHCDR3 amino acid sequence of SEQ ID NO: 139; and

(ii) a light chain variable region (VL) comprising a VLCDR1 amino acid sequence of SEQ ID NO: 146, a VLCDR2 amino acid sequence of SEQ ID NO: 147, and a VLCDR3 amino acid sequence of SEQ ID NO: 166.

In other embodiments, the anti-PD-1 antibody molecule comprises:

(i) a heavy chain variable region (VH) comprising a VHCDR1 amino acid sequence chosen from SEQ ID NO: 137, SEQ ID NO: 140 or SEQ ID NO: 286; a VHCDR2 amino acid sequence of SEQ ID NO: 141, and a VHCDR3 amino acid sequence of SEQ ID NO: 139; and

(ii) a light chain variable region (VL) comprising a VLCDR1 amino acid sequence of SEQ ID NO: 149, a VLCDR2 amino acid sequence of SEQ ID NO: 150, and a VLCDR3 amino acid sequence of SEQ ID NO: 167.

In embodiments of the aforesaid antibody molecules, the VHCDR1 comprises the amino acid sequence of SEQ ID NO: 137. In other embodiments, the VHCDR1 comprises the amino acid sequence of SEQ ID NO: 140. In yet other embodiments, the VHCDR1 amino acid sequence of SEQ ID NO: 286.

In embodiments, the aforesaid antibody molecules have a heavy chain variable region comprising at least one framework (FW) region comprising the amino acid sequence of any of SEQ ID NOs: 147, 151, 153, 157, 160, 162, 166, or 169 of US 2015/0210769A1, or an amino acid sequence at least 90% identical thereto, or having no more than two amino acid substitutions, insertions or deletions compared to the amino acid sequence of any of SEQ ID NOs: 147, 151, 153, 157, 160, 162, 166, or 169 of US 2015/0210769A1.

In other embodiments, the aforesaid antibody molecules have a heavy chain variable region comprising at least one framework region comprising the amino acid sequence of any of SEQ ID NOs: 147, 151, 153, 157, 160, 162, 166, or 169 of US 2015/0210769A1.

In yet other embodiments, the aforesaid antibody molecules have a heavy chain variable region comprising at least two, three, or four framework regions comprising the amino acid sequences of any of SEQ ID NOs: 147, 151, 153, 157, 160, 162, 166, or 169 of US 2015/0210769A1.

In other embodiments, the aforesaid antibody molecules comprise a VHFW1 amino acid sequence of SEQ ID NO: 147 or 151 of US 2015/0210769A1, a VHFW2 amino acid sequence of SEQ ID NO: 153, 157, or 160 of US 2015/0210769A1, and a VHFW3 amino acid sequence of SEQ ID NO: 162 or 166 of US 2015/0210769A1, and, optionally, further comprising a VHFW4 amino acid sequence of SEQ ID NO: 169 of US 2015/0210769A1.

In other embodiments, the aforesaid antibody molecules have a light chain variable region comprising at least one framework region comprising the amino acid sequence of any of SEQ ID NOs: 174, 177, 181, 183, 185, 187, 191, 194, 196, 200, 202, 205, or 208 of US 2015/0210769A1, or an amino acid sequence at least 90% identical thereto, or having no more than two amino acid substitutions, insertions or deletions compared to the amino acid sequence of any of 174, 177, 181, 183, 185, 187, 191, 194, 196, 200, 202, 205, or 208 of US 2015/0210769A1.

In other embodiments, the aforesaid antibody molecules have a light chain variable region comprising at least one framework region comprising the amino acid sequence of any of SEQ ID NOs: 174, 177, 181, 183, 185, 187, 191, 194, 196, 200, 202, 205, or 208 of US 2015/0210769A1.

In other embodiments, the aforesaid antibody molecules have a light chain variable region comprising at least two, three, or four framework regions comprising the amino acid sequences of any of SEQ ID NOs: 174, 177, 181, 183, 185, 187, 191, 194, 196, 200, 202, 205, or 208 of US 2015/0210769A1.

In other embodiments, the aforesaid antibody molecules comprise a VLFW1 amino acid sequence of SEQ ID NO: 174, 177, 181, 183, or 185 of US 2015/0210769A1, a VLFW2 amino acid sequence of SEQ ID NO: 187, 191, or 194 of US 2015/0210769A1, and a VLFW3 amino acid sequence of SEQ ID NO: 196, 200, 202, or 205 of US 2015/0210769A1, and, optionally, further comprising a VLFW4 amino acid sequence of SEQ ID NO: 208 of US 2015/0210769A1.

In other embodiments, the aforesaid antibodies comprise a heavy chain variable domain comprising an amino acid sequence at least 85% identical to any of SEQ ID NOs: 172, 184, 216, or 220.

In other embodiments, the aforesaid antibody molecules comprise a heavy chain variable domain comprising the amino acid sequence of SEQ ID NO: 172, 184, 216, or 220.

In other embodiments, the aforesaid antibody molecules comprise a light chain variable domain comprising an amino acid sequence at least 85% identical to any of SEQ ID NOs: 176, 180, 188, 192, 196, 200, 204, 208, or 212.

In other embodiments, the aforesaid antibody molecules comprise a light chain variable domain comprising the amino acid sequence of SEQ ID NO: 176, 180, 188, 192, 196, 200, 204, 208, or 212.

In other embodiments, the aforesaid antibody molecules comprise a heavy chain variable domain comprising the amino acid sequence of SEQ ID NO: 172.

In other embodiments, the aforesaid antibody molecules comprise a heavy chain comprising the amino acid sequence of SEQ ID NO: 174.

In other embodiments, the aforesaid antibody molecules comprise a heavy chain comprising the amino acid sequence of SEQ ID NO: 225.

In other embodiments, the aforesaid antibody molecules comprise a heavy chain variable domain comprising the amino acid sequence of SEQ ID NO: 184.

In other embodiments, the aforesaid antibody molecules comprise a heavy chain comprising the amino acid sequence of SEQ ID NO: 186 or SEQ ID NO: 236.

In other embodiments, the aforesaid antibody molecules comprise a heavy chain variable domain comprising the amino acid sequence of SEQ ID NO: 216.

In other embodiments, the aforesaid antibody molecules comprise a heavy chain comprising the amino acid sequence of SEQ ID NO: 218.

In other embodiments, the aforesaid antibody molecules comprise a heavy chain variable domain comprising the amino acid sequence of SEQ ID NO: 220.

In other embodiments, the aforesaid antibody molecules comprise a heavy chain comprising the amino acid sequence of SEQ ID NO: 222.

In other embodiments, the aforesaid antibody molecules comprise a light chain variable domain comprising the amino acid sequence of SEQ ID NO: 176.

In other embodiments, the aforesaid antibody molecules comprise a light chain comprising the amino acid sequence of SEQ ID NO: 178.

In other embodiments, the aforesaid antibody molecules comprise a light chain variable domain comprising the amino acid sequence of SEQ ID NO: 180.

In other embodiments, the aforesaid antibody molecules comprise a light chain comprising the amino acid sequence of SEQ ID NO: 182.

In other embodiments, the aforesaid antibody molecules comprise a light chain variable domain comprising the amino acid sequence of SEQ ID NO: 188.

In other embodiments, the aforesaid antibody molecules comprise a light chain comprising the amino acid sequence of SEQ ID NO: 190.

In other embodiments, the aforesaid antibody molecules comprise a light chain variable domain comprising the amino acid sequence of SEQ ID NO: 192.

In other embodiments, the aforesaid antibody molecules comprise a light chain comprising the amino acid sequence of SEQ ID NO: 194.

In other embodiments, the aforesaid antibody molecules comprise a light chain variable domain comprising the amino acid sequence of SEQ ID NO: 196.

In other embodiments, the aforesaid antibodies comprise a light chain comprising the amino acid sequence of SEQ ID NO: 198.

In other embodiments, the aforesaid antibody molecules comprise a light chain variable domain comprising the amino acid sequence of SEQ ID NO: 200.

In other embodiments, the aforesaid antibody molecules comprise a light chain comprising the amino acid sequence of SEQ ID NO: 202.

In other embodiments, the aforesaid antibody molecules comprise a light chain variable domain comprising the amino acid sequence of SEQ ID NO: 204.

In other embodiments, the aforesaid antibody molecules comprise a light chain comprising the amino acid sequence of SEQ ID NO: 206.

In other embodiments, the aforesaid antibody molecules comprise a light chain variable domain comprising the amino acid sequence of SEQ ID NO: 208.

In other embodiments, the aforesaid antibody molecules comprise a light chain comprising the amino acid sequence of SEQ ID NO: 210.

In other embodiments, the aforesaid antibody molecules comprise a light chain variable domain comprising the amino acid sequence of SEQ ID NO: 212.

In other embodiments, the aforesaid antibody molecules comprise a light chain comprising the amino acid sequence of SEQ ID NO: 214.

In other embodiments, the aforesaid antibody molecules comprise a heavy chain variable domain comprising the amino acid sequence of SEQ ID NO: 172 and a light chain variable domain comprising the amino acid sequence of SEQ ID NO: 176.

In other embodiments, the aforesaid antibody molecules comprise a heavy chain variable domain comprising the amino acid sequence of SEQ ID NO: 172 and a light chain variable domain comprising the amino acid sequence of SEQ ID NO: 200.

In other embodiments, the aforesaid antibody molecules comprise a heavy chain variable domain comprising the amino acid sequence of SEQ ID NO: 172 and a light chain variable domain comprising the amino acid sequence of SEQ ID NO: 204.

In other embodiments, the aforesaid antibody molecules comprise a heavy chain variable domain comprising the amino acid sequence of SEQ ID NO: 184 and a light chain variable domain comprising the amino acid sequence of SEQ ID NO: 204.

In other embodiments, the aforesaid antibody molecules comprise a heavy chain variable domain comprising the amino acid sequence of SEQ ID NO: 172 and a light chain variable domain comprising the amino acid sequence of SEQ ID NO: 180.

In other embodiments, the aforesaid antibody molecules comprise a heavy chain variable domain comprising the amino acid sequence of SEQ ID NO: 184 and a light chain variable domain comprising the amino acid sequence of SEQ ID NO: 180.

In other embodiments, the aforesaid antibody molecules comprise a heavy chain variable domain comprising the amino acid sequence of SEQ ID NO: 184 and a light chain variable domain comprising the amino acid sequence of SEQ ID NO: 188.

In other embodiments, the aforesaid antibody molecules comprise a heavy chain variable domain comprising the amino acid sequence of SEQ ID NO: 172 and a light chain variable domain comprising the amino acid sequence of SEQ ID NO: 188.

In other embodiments, the aforesaid antibody molecules comprise a heavy chain variable domain comprising the amino acid sequence of SEQ ID NO: 172 and a light chain variable domain comprising the amino acid sequence of SEQ ID NO: 192.

In other embodiments, the aforesaid antibody molecules comprise a heavy chain variable domain comprising the amino acid sequence of SEQ ID NO: 172 and a light chain variable domain comprising the amino acid sequence of SEQ ID NO: 196.

In other embodiments, the aforesaid antibody molecules comprise a heavy chain variable domain comprising the amino acid sequence of SEQ ID NO: 184 and a light chain variable domain comprising the amino acid sequence of SEQ ID NO: 200.

In other embodiments, the aforesaid antibody molecules comprise a heavy chain variable domain comprising the amino acid sequence of SEQ ID NO: 172 and a light chain variable domain comprising the amino acid sequence of SEQ ID NO: 208.

In other embodiments, the aforesaid antibody molecules comprise a heavy chain variable domain comprising the amino acid sequence of SEQ ID NO: 172 and a light chain variable domain comprising the amino acid sequence of SEQ ID NO: 212.

In other embodiments, the aforesaid antibody molecules comprise a heavy chain variable domain comprising the amino acid sequence of SEQ ID NO: 216 and a light chain variable domain comprising the amino acid sequence of SEQ ID NO: 204.

In other embodiments, the aforesaid antibody molecules comprise a heavy chain variable domain comprising the amino acid sequence of SEQ ID NO: 216 and a light chain variable domain comprising the amino acid sequence of SEQ ID NO: 200.

In other embodiments, the aforesaid antibody molecules comprise a heavy chain variable domain comprising the amino acid sequence of SEQ ID NO: 220 and a light chain variable domain comprising the amino acid sequence of SEQ ID NO: 200.

In other embodiments, the aforesaid antibody molecules comprise a heavy chain comprising the amino acid sequence of SEQ ID NO: 225 and a light chain comprising the amino acid sequence of SEQ ID NO: 178.

In other embodiments, the aforesaid antibody molecules comprise a heavy chain comprising the amino acid sequence of SEQ ID NO: 225 and a light chain comprising the amino acid sequence of SEQ ID NO: 190.

In other embodiments, the aforesaid antibody molecules comprise a heavy chain comprising the amino acid sequence of SEQ ID NO: 225 and a light chain comprising the amino acid sequence of SEQ ID NO: 202.

In other embodiments, the aforesaid antibody molecules comprise a heavy chain comprising the amino acid sequence of SEQ ID NO: 225 and a light chain comprising the amino acid sequence of SEQ ID NO: 206.

In other embodiments, the aforesaid antibody molecules comprise a heavy chain comprising the amino acid sequence of SEQ ID NO: 236 and a light chain comprising the amino acid sequence of SEQ ID NO: 206.

In other embodiments, the aforesaid antibody molecules comprise a heavy chain comprising the amino acid sequence of SEQ ID NO: 174 and a light chain comprising the amino acid sequence of SEQ ID NO: 178.

In other embodiments, the aforesaid antibody molecules comprise a heavy chain comprising the amino acid sequence of SEQ ID NO: 174 and a light chain comprising the amino acid sequence of SEQ ID NO: 182.

In other embodiments, the aforesaid antibody molecules comprise a heavy chain comprising the amino acid sequence of SEQ ID NO: 186 and a light chain comprising the amino acid sequence of SEQ ID NO: 182.

In other embodiments, the aforesaid antibody molecules comprise a heavy chain comprising the amino acid sequence of SEQ ID NO: 186 and a light chain comprising the amino acid sequence of SEQ ID NO: 190.

In other embodiments, the aforesaid antibody molecules comprise a heavy chain comprising the amino acid sequence of SEQ ID NO: 174 and a light chain comprising the amino acid sequence of SEQ ID NO: 190.

In other embodiments, the aforesaid antibodies comprise a heavy chain comprising the amino acid sequence of SEQ ID NO: 174 and a light chain comprising the amino acid sequence of SEQ ID NO: 194.

In other embodiments, the aforesaid antibody molecules comprise a heavy chain comprising the amino acid sequence of SEQ ID NO: 174 and a light chain comprising the amino acid sequence of SEQ ID NO: 198.

In other embodiments, the aforesaid antibody molecules comprise a heavy chain comprising the amino acid sequence of SEQ ID NO: 186 and a light chain comprising the amino acid sequence of SEQ ID NO: 202.

In other embodiments, the aforesaid antibody molecules comprise a heavy chain comprising the amino acid sequence of SEQ ID NO: 174 and a light chain comprising the amino acid sequence of SEQ ID NO: 202.

In other embodiments, the aforesaid antibody molecules comprise a heavy chain comprising the amino acid sequence of SEQ ID NO: 186 and a light chain comprising the amino acid sequence of SEQ ID NO: 206.

In other embodiments, the aforesaid antibody molecules comprise a heavy chain comprising the amino acid sequence of SEQ ID NO: 174 and a light chain comprising the amino acid sequence of SEQ ID NO: 206.

In other embodiments, the aforesaid antibody molecules comprise a heavy chain comprising the amino acid sequence of SEQ ID NO: 174 and a light chain comprising the amino acid sequence of SEQ ID NO: 210.

In other embodiments, the aforesaid antibody molecules comprise a heavy chain comprising the amino acid sequence of SEQ ID NO: 174 and a light chain comprising the amino acid sequence of SEQ ID NO: 214.

In other embodiments, the aforesaid antibody molecules comprise a heavy chain comprising the amino acid sequence of SEQ ID NO: 218 and a light chain comprising the amino acid sequence of SEQ ID NO: 206.

In other embodiments, the aforesaid antibodies comprise a heavy chain comprising the amino acid sequence of SEQ ID NO: 218 and a light chain comprising the amino acid sequence of SEQ ID NO: 202.

In other embodiments, the aforesaid antibody molecules comprise a heavy chain comprising the amino acid sequence of SEQ ID NO: 222 and a light chain comprising the amino acid sequence of SEQ ID NO: 202.

In other embodiments, the aforesaid antibody molecules are chosen from a Fab, F(ab′)2, Fv, or a single chain Fv fragment (scFv).

In other embodiments, the aforesaid antibody molecules comprise a heavy chain constant region selected from IgG1, IgG2, IgG3, and IgG4.

In other embodiments, the aforesaid antibody molecules comprise a light chain constant region chosen from the light chain constant regions of kappa or lambda.

In other embodiments, the aforesaid antibody molecules comprise a human IgG4 heavy chain constant region with a mutation at position 228 according to EU numbering or position 108 of SEQ ID NO: 212 or 214 of US 2015/0210769A1 and a kappa light chain constant region.

In other embodiments, the aforesaid antibody molecules comprise a human IgG4 heavy chain constant region with a Serine to Proline mutation at position 228 according to EU numbering or position 108 of SEQ ID NO: 212 or 214 of US 2015/0210769A1 and a kappa light chain constant region.

In other embodiments, the aforesaid antibody molecules comprise a human IgG1 heavy chain constant region with an Asparagine to Alanine mutation at position 297 according to EU numbering or position 180 of SEQ ID NO: 216 of US 2015/0210769A1 and a kappa light chain constant region.

In other embodiments, the aforesaid antibody molecules comprise a human IgG1 heavy chain constant region with an Aspartate to Alanine mutation at position 265 according to EU numbering or position 148 of SEQ ID NO: 217 of US 2015/0210769A1, and Proline to Alanine mutation at position 329 according to EU numbering or position 212 of SEQ ID NO: 217 of US 2015/0210769A1 and a kappa light chain constant region.

In other embodiments, the aforesaid antibody molecules comprise a human IgG1 heavy chain constant region with a Leucine to Alanine mutation at position 234 according to EU numbering or position 117 of SEQ ID NO: 218 of US 2015/0210769A1, and Leucine to Alanine mutation at position 235 according to EU numbering or position 118 of SEQ ID NO: 218 of US 2015/0210769A1 and a kappa light chain constant region.

In other embodiments, the aforesaid antibody molecules are capable of binding to human PD-1 with a dissociation constant (K_{D}) of less than about 0.2 nM.

In some embodiments, the aforesaid antibody molecules bind to human PD-1 with a K_{D} of less than about 0.2 nM, 0.15 nM, 0.1 nM, 0.05 nM, or 0.02 nM, e.g., about 0.13 nM to 0.03 nM, e.g., about 0.077 nM to 0.088 nM, e.g., about 0.083 nM, e.g., as measured by a Biacore method.

In other embodiments, the aforesaid antibody molecules bind to cynomolgus PD-1 with a K_{D} of less than about 0.2 nM, 0.15 nM, 0.1 nM, 0.05 nM, or 0.02 nM, e.g., about 0.11 nM to 0.08 nM, e.g., about 0.093 nM, e.g., as measured by a Biacore method.

In certain embodiments, the aforesaid antibody molecules bind to both human PD-1 and cynomolgus PD-1 with similar K_{D}, e.g., in the nM range, e.g., as measured by a Biacore method. In some embodiments, the aforesaid antibody molecules bind to a human PD-1-Ig fusion protein with a K_{D} of less than about 0.1 nM, 0.075 nM, 0.05 nM, 0.025 nM, or 0.01 nM, e.g., about 0.04 nM, e.g., as measured by ELISA.

In some embodiments, the aforesaid antibody molecules bind to Jurkat cells that express human PD-1 (e.g., human PD-1-transfected Jurkat cells) with a K_{D} of less than about 0.1 nM, 0.075 nM, 0.05 nM, 0.025 nM, or 0.01 nM, e.g., about 0.06 nM, e.g., as measured by FACS analysis.

In some embodiments, the aforesaid antibody molecules bind to cynomolgus T cells with a K_{D} of less than about 1 nM, 0.75 nM, 0.5 nM, 0.25 nM, or 0.1 nM, e.g., about 0.4 nM, e.g., as measured by FACS analysis.

In some embodiments, the aforesaid antibody molecules bind to cells that express cynomolgus PD-1 (e.g., cells transfected with cynomolgus PD-1) with a K_{D} of less than about 1 nM, 0.75 nM, 0.5 nM, 0.25 nM, or 0.01 nM, e.g., about 0.6 nM, e.g., as measured by FACS analysis.

In certain embodiments, the aforesaid antibody molecules are not cross-reactive with mouse or rat PD-1. In other embodiments, the aforesaid antibodies are cross-reactive with rhesus PD-1. For example, the cross-reactivity can be measured by a Biacore method or a binding assay using cells that expresses PD-1 (e.g., human PD-1-expressing 300.19 cells). In other embodiments, the aforesaid antibody molecules bind an extracellular Ig-like domain of PD-1.

In other embodiments, the aforesaid antibody molecules are capable of reducing binding of PD-1 to PD-L1, PD-L2, or both, or a cell that expresses PD-L1, PD-L2, or both. In some embodiments, the aforesaid antibody molecules reduce (e.g., block) PD-L1 binding to a cell that expresses PD-1 (e.g., human PD-1-expressing 300.19 cells) with an IC50 of less than about 1.5 nM, 1 nM, 0.8 nM, 0.6 nM, 0.4 nM, 0.2 nM, or 0.1 nM, e.g., between about 0.79 nM and about 1.09 nM, e.g., about 0.94 nM, or about 0.78 nM or less, e.g., about 0.3 nM. In some embodiments, the aforesaid antibodies reduce (e.g., block) PD-L2 binding to a cell that expresses PD-1 (e.g., human PD-1-expressing 300.19 cells) with an IC50 of less than about 2 nM, 1.5 nM, 1 nM, 0.5 nM, or 0.2 nM, e.g., between about 1.05 nM and about 1.55 nM, or about 1.3 nM or less, e.g., about 0.9 nM.

In other embodiments, the aforesaid antibody molecules are capable of enhancing an antigen-specific T cell response.

In embodiments, the antibody molecule is a monospecific antibody molecule or a bispecific antibody molecule. In embodiments, the antibody molecule has a first binding specificity for PD-1 and a second binding specifity for TIM-3, LAG-3, CEACAM (e.g., CEACAM-1, CEACAM-3, and/or CEACAM-5), PD-L1 or PD-L2. In embodiments, the antibody molecule comprises an antigen binding fragment of an antibody, e.g., a half antibody or antigen binding fragment of a half antibody.

In some embodiments, the aforesaid antibody molecules increase the expression of IL-2 from cells activated by Staphylococcal enterotoxin B (SEB) (e.g., at 25 μg/mL) by at least about 2, 3, 4, 5-fold, e.g., about 2 to 3-fold, e.g., about 2 to 2.6-fold, e.g., about 2.3-fold, compared to the expression of IL-2 when an isotype control (e.g., IgG4) is used, e.g., as measured in a SEB T cell activation assay or a human whole blood ex vivo assay.

In some embodiments, the aforesaid antibody molecules increase the expression of IFN-γ from T cells stimulated by anti-CD3 (e.g., at 0.1 μg/mL) by at least about 2, 3, 4, 5-fold, e.g., about 1.2 to 3.4-fold, e.g., about 2.3-fold, compared to the expression of IFN-γ when an isotype control (e.g., IgG4) is used, e.g., as measured in an IFN-γ activity assay.

In some embodiments, the aforesaid antibody molecules increase the expression of IFN-γ from T cells activated by SEB (e.g., at 3 pg/mL) by at least about 2, 3, 4, 5-fold, e.g., about 0.5 to 4.5-fold, e.g., about 2.5-fold, compared to the expression of IFN-γ when an isotype control (e.g., IgG4) is used, e.g., as measured in an IFN-γ activity assay.

In some embodiments, the aforesaid antibody molecules increase the expression of IFN-γ from T cells activated with an CMV peptide by at least about 2, 3, 4, 5-fold, e.g., about 2 to 3.6-fold, e.g., about 2.8-fold, compared to the expression of IFN-γ when an isotype control (e.g., IgG4) is used, e.g., as measured in an IFN-γ activity assay.

In some embodiments, the aforesaid antibody molecules increase the proliferation of CD8⁺ T cells activated with an CMV peptide by at least about 1, 2, 3, 4, 5-fold, e.g., about 1.5-fold, compared to the proliferation of CD8⁺ T cells when an isotype control (e.g., IgG4) is used, e.g., as measured by the percentage of CD8+ T cells that passed through at least n (e.g., n=2 or 4) cell divisions.

In certain embodiments, the aforesaid antibody molecules has a Cmax between about 100 μg/mL and about 500 μg/mL, between about 150 μg/mL and about 450 μg/mL, between about 250 μg/mL and about 350 μg/mL, or between about 200 μg/mL and about 400 μg/mL, e.g., about 292.5 μg/mL, e.g., as measured in monkey.

In certain embodiments, the aforesaid antibody molecules has a T₁₁₂ between about 250 hours and about 650 hours, between about 300 hours and about 600 hours, between about 350 hours and about 550 hours, or between about 400 hours and about 500 hours, e.g., about 465.5 hours, e.g., as measured in monkey.

In some embodiments, the aforesaid antibody molecules bind to PD-1 with a Kd slower than 5×10⁻⁴, 1×10⁻⁴, 5×10⁻⁵, or 1×10⁻⁵ s⁻¹, e.g., about 2.13×10⁻⁴ s⁻¹, e.g., as measured by a Biacore method. In some embodiments, the aforesaid antibody molecules bind to PD-1 with a Ka faster than 1×10⁴, 5×10⁴, 1×10⁵, or 5×10⁵ M⁻¹s⁻¹, e.g., about 2.78×10⁵ M⁻¹s⁻¹, e.g., as measured by a Biacore method.

In some embodiments, the aforesaid anti-PD-1 antibody molecules bind to one or more residues within the C strand, CC′ loop, C′ strand and FG loop of PD-1. The domain structure of PD-1 is described, e.g., in Cheng et al., “Structure and Interactions of the Human Programmed Cell Death 1 Receptor” J. Biol. Chem. 2013, 288:11771-11785. As described in Cheng et. al., the C strand comprises residues F43-M50, the CC′ loop comprises S51-N54, the C′ strand comprises residues Q55-F62, and the FG loop comprises residues L108-I114 (amino acid numbering according to Chang et al. supra). Accordingly, in some embodiments, an anti-PD-1 antibody as described herein binds to at least one residue in one or more of the ranges F43-M50, S51-N54, Q55-F62, and L108-I114 of PD-1. In some embodiments, an anti-PD-1 antibody as described herein binds to at least one residue in two, three, or all four of the ranges F43-M50, S51-N54, Q55-F62, and L108-I114 of PD-1. In some embodiments, the anti-PD-1 antibody binds to a residue in PD-1 that is also part of a binding site for one or both of PD-L1 and PD-L2.

In another aspect, the invention provides an isolated nucleic acid molecule encoding any of the aforesaid antibody molecules, vectors and host cells thereof.

An isolated nucleic acid encoding the antibody heavy chain variable region or light chain variable region, or both, of any the aforesaid antibody molecules is also provided.

In one embodiment, the isolated nucleic acid encodes heavy chain CDRs 1-3, wherein said nucleic acid comprises a nucleotide sequence of SEQ ID NO: 242-246, 255, 256-260, 267-271, or 278-280.

In another embodiment, the isolated nucleic acid encodes light chain CDRs 1-3, wherein said nucleic acid comprises a nucleotide sequence of SEQ ID NO: 247-254, 261-266, or 272-277.

In other embodiments, the aforesaid nucleic acid further comprises a nucleotide sequence encoding a heavy chain variable domain, wherein said nucleotide sequence is at least 85% identical to any of SEQ ID NO: 173, 185, 217, 221, 224, 229, or 235.

In other embodiments, the aforesaid nucleic acid further comprises a nucleotide sequence encoding a heavy chain variable domain, wherein said nucleotide sequence comprises any of SEQ ID NO: 173, 185, 217, 221, 224, 229, or 235.

In other embodiments, the aforesaid nucleic acid further comprises a nucleotide sequence encoding a heavy chain, wherein said nucleotide sequence is at least 85% identical to any of SEQ ID NO: 175, 187, 219, 223, 226, 230, or 237.

In other embodiments, the aforesaid nucleic acid further comprises a nucleotide sequence encoding a heavy chain, wherein said nucleotide sequence comprises any of SEQ ID NO: 175, 187, 219, 223, 226, 230, or 237.

In other embodiments, the aforesaid nucleic acid further comprises a nucleotide sequence encoding a light chain variable domain, wherein said nucleotide sequence is at least 85% identical to any of SEQ ID NO: 177, 181, 189, 193, 197, 201, 205, 209, 213, 227, 231, 233, 238, or 240.

In other embodiments, the aforesaid nucleic acid further comprises a nucleotide sequence encoding a light chain variable domain, wherein said nucleotide sequence comprises any of SEQ ID NO: 177, 181, 189, 193, 197, 201, 205, 209, 213, 227, 231, 233, 238, or 240.

In other embodiments, the aforesaid nucleic acid further comprises a nucleotide sequence encoding a light chain, wherein said nucleotide sequence is at least 85% identical to any of SEQ ID NO: 179, 183, 191, 195, 199, 203, 207, 211, 215, 228, 232, 234, 239 or 241.

In other embodiments, the aforesaid nucleic acid further comprises a nucleotide sequence encoding a light chain, wherein said nucleotide sequence comprises any of SEQ ID NO: 179, 183, 191, 195, 199,203,207, 211, 215, 228, 232, 234, 239 or 241.

In certain embodiments, one or more expression vectors and host cells comprising the aforesaid nucleic acids are provided.

A method of producing an antibody molecule or fragment thereof, comprising culturing the host cell as described herein under conditions suitable for gene expression is also provided.

In one aspect, the disclosure features a method of providing an antibody molecule described herein. The method includes: providing a PD-1 antigen (e.g., an antigen comprising at least a portion of a PD-1 epitope); obtaining an antibody molecule that specifically binds to the PD-1 polypeptide; and evaluating if the antibody molecule specifically binds to the PD-1 polypeptide, or evaluating efficacy of the antibody molecule in modulating, e.g., inhibiting, the activity of the PD-1. The method can further include administering the antibody molecule to a subject, e.g., a human or non-human animal.

In another aspect, the disclosure provides compositions, e.g., pharmaceutical compositions, which include a pharmaceutically acceptable carrier, excipient or stabilizer, and at least one of the therapeutic agents, e.g., anti-PD-1 antibody molecules described herein. In one embodiment, the composition, e.g., the pharmaceutical composition, includes a combination of the antibody molecule and one or more agents, e.g., a therapeutic agent or other antibody molecule, as described herein. In one embodiment, the antibody molecule is conjugated to a label or a therapeutic agent.

TABLE 6
Amino acid and nucleotide sequences for murine, chimeric and humanized anti-PD-1
antibody molecules. The antibody molecules include murine mAb BAP049, chimeric mAbs
BAP049-chi and BAP049-chi-Y, and humanized mAbs BAP049-hum01 to BAP049-hum16 and
BAP049-Clone-A to BAP049-Clone-E. The amino acid and nucleotide sequences of the heavy
and light chain CDRs, the heavy and light chain variable regions, and the heavy and
light chains are shown.
BAP049 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 142 | VH | QVQLQQPGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTLVTVSA |
| SEQ ID NO: 143 | DNA VH | CAGGTCCAGCTGCAGCAACCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAAGGG |
| | | ACTCTGGTCACTGTCTCTGCA |
| SEQ ID NO: 144 | VH | QVQLQQSGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTLVTVSA |
| SEQ ID NO: 145 | DNA VH | CAGGTCCAGCTGCAGCAGTCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAAGGG |
| | | ACTCTGGTCACTGTCTCTGCA |

BAP049 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 148 (Kabat) | LCDR3 | QNDYSYPCT |
| SEQ ID NO: 149 (Chothia) | LCDR1 | SQSLLDSGNQKNF |
| SEQ ID NO: 150 (Chothia) | LCDR2 | WAS |
| SEQ ID NO: 151 (Chothia) | LCDR3 | DYSYPC |
| SEQ ID NO: 152 | VL | DIVMTQSPSSLTVTAGEKVTMSCKSSQSLLDSG |
| | | NQKNFLTWYQQKPGQPPKLLIFWASTRESGVPD |
| | | RFTGSGSVTDFTLTISSVQAEDLAVYYCQNDYS |
| | | YPCTFGGGTKLEIK |
| SEQ ID NO: 153 | DNA VL | GACATTGTGATGACCCAGTCTCCATCCTCCCTG |
| | | ACTGTGACAGCAGGAGAGAAGGTCACTATGAGC |
| | | TGCAAGTCCAGTCAGAGTCTGTTAGACAGTGGA |
| | | AATCAAAAGAACTTCTTGACCTGGTACCAGCAG |
| | | AAACCAGGGCAGCCTCCTAAACTGTTGATCTTC |
| | | TGGGCATCCACTAGGGAATCTGGGGTCCCTGAT |
| | | CGCTTCACAGGCAGTGGATCTGTAACAGATTTC |
| | | ACTCTCACCATCAGCAGTGTGCAGGCTGAAGAC |
| | | CTGGCAGTTTATTACTGTCAGAATGATTATAGT |
| | | TATCCGTGCACGTTCGGAGGGGGGACCAAGCTG |
| | | GAAATAAAA |

BAP049-chi HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 154 | VH | QVQLQQPGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSS |
| SEQ ID NO: 155 | DNA VH | CAGGTCCAGCTGCAGCAGCCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCC |
| SEQ ID NO: 156 | HC | QVQLQQPGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 157 | DNA HC | CAGGTCCAGCTGCAGCAGCCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| SEQ ID NO: 158 | VH | QVQLQQSGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSS |
| SEQ ID NO: 159 | DNA VH | CAGGTCCAGCTGCAGCAGTCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCC |
| SEQ ID NO: 160 | HC | QVQLQQSGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 161 | DNA HC | CAGGTCCAGCTGCAGCAGTCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-chi LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 148 (Kabat) | LCDR3 | QNDYSYPCT |
| SEQ ID NO: 149 (Chothia) | LCDR1 | SQSLLDSGNQKNF |
| SEQ ID NO: 150 (Chothia) | LCDR2 | WAS |
| SEQ ID NO: 151 (Chothia) | LCDR3 | DYSYPC |
| SEQ ID NO: 162 | VL | DIVMTQSPSSLTVTAGEKVTMSCKSSQSLLDSG |
| | | NQKNFLTWYQQKPGQPPKLLIFWASTRESGVPD |
| | | RFTGSGSVTDFTLTISSVQAEDLAVYYCQNDYS |
| | | YPCTFGQGTKVEIK |
| SEQ ID NO: 163 | DNA VL | GACATTGTGATGACCCAGTCTCCATCCTCCCTG |
| | | ACTGTGACAGCAGGAGAGAAGGTCACTATGAGC |
| | | TGCAAGTCCAGTCAGAGTCTGTTAGACAGTGGA |
| | | AATCAAAAGAACTTCTTGACCTGGTACCAGCAG |
| | | AAACCAGGGCAGCCTCCTAAACTGTTGATCTTC |
| | | TGGGCATCCACTAGGGAATCTGGGGTCCCTGAT |
| | | CGCTTCACAGGCAGTGGATCTGTAACAGATTTC |
| | | ACTCTCACCATCAGCAGTGTGCAGGCTGAAGAC |
| | | CTGGCAGTTTATTACTGTCAGAATGATTATAGT |
| | | TATCCGTGCACGTTCGGCCAAGGGACCAAGGTG |
| | | GAAATCAAA |
| SEQ ID NO: 164 | LC | DIVMTQSPSSLTVTAGEKVTMSCKSSQSLLDSG |
| | | NQKNFLTWYQQKPGQPPKLLIFWASTRESGVPD |
| | | RFTGSGSVTDFTLTISSVQAEDLAVYYCQNDYS |
| | | YPCTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 165 | DNA LC | GACATTGTGATGACCCAGTCTCCATCCTCCCTG |
| | | ACTGTGACAGCAGGAGAGAAGGTCACTATGAGC |
| | | TGCAAGTCCAGTCAGAGTCTGTTAGACAGTGGA |
| | | AATCAAAAGAACTTCTTGACCTGGTACCAGCAG |
| | | AAACCAGGGCAGCCTCCTAAACTGTTGATCTTC |
| | | TGGGCATCCACTAGGGAATCTGGGGTCCCTGAT |
| | | CGCTTCACAGGCAGTGGATCTGTAACAGATTTC |
| | | ACTCTCACCATCAGCAGTGTGCAGGCTGAAGAC |
| | | CTGGCAGTTTATTACTGTCAGAATGATTATAGT |
| | | TATCCGTGCACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-chi-Y HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 154 | VH | QVQLQQPGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSS |
| SEQ ID NO: 155 | DNA VH | CAGGTCCAGCTGCAGCAGCCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCC |
| SEQ ID NO: 156 | HC | QVQLQQPGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 157 | DNA HC | CAGGTCCAGCTGCAGCAGCCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |
| SEQ ID NO: 158 | VH | QVQLQQSGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSS |
| SEQ ID NO: 159 | DNA VH | CAGGTCCAGCTGCAGCAGTCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCC |
| SEQ ID NO: 160 | HC | QVQLQQSGSELVRPGASVKLSCKASGYTFTTYW |
| | | MHWVRQRPGQGLEWIGNIYPGTGGSNFDEKFKN |
| | | RTSLTVDTSSTTAYMHLASLTSEDSAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 161 | DNA HC | CAGGTCCAGCTGCAGCAGTCTGGGTCTGAGCTG |
| | | GTGAGGCCTGGAGCTTCAGTGAAGCTGTCCTGC |
| | | AAGGCGTCTGGCTACACATTCACCACTTACTGG |
| | | ATGCACTGGGTGAGGCAGAGGCCTGGACAAGGC |
| | | CTTGAGTGGATTGGAAATATTTATCCTGGTACT |
| | | GGTGGTTCTAACTTCGATGAGAAGTTCAAAAAC |
| | | AGGACCTCACTGACTGTAGACACATCCTCCACC |
| | | ACAGCCTACATGCACCTCGCCAGCCTGACATCT |
| | | GAGGACTCTGCGGTCTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-chi-Y LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 168 | VL | DIVMTQSPSSLTVTAGEKVTMSCKSSQSLLDSG |
| | | NQKNFLTWYQQKPGQPPKLLIFWASTRESGVPD |
| | | RFTGSGSVTDFTLTISSVQAEDLAVYYCQNDYS |
| | | YPYTFGQGTKVEIK |
| SEQ ID NO: 169 | DNA VL | GACATTGTGATGACCCAGTCTCCATCCTCCCTG |
| | | ACTGTGACAGCAGGAGAGAAGGTCACTATGAGC |
| | | TGCAAGTCCAGTCAGAGTCTGTTAGACAGTGGA |
| | | AATCAAAAGAACTTCTTGACCTGGTACCAGCAG |
| | | AAACCAGGGCAGCCTCCTAAACTGTTGATCTTC |
| | | TGGGCATCCACTAGGGAATCTGGGGTCCCTGAT |
| | | CGCTTCACAGGCAGTGGATCTGTAACAGATTTC |
| | | ACTCTCACCATCAGCAGTGTGCAGGCTGAAGAC |
| | | CTGGCAGTTTATTACTGTCAGAATGATTATAGT |
| | | TATCCGTACACGTTCGGCCAAGGGACCAAGGTG |
| | | GAAATCAAA |
| SEQ ID NO: 170 | LC | DIVMTQSPSSLTVTAGEKVTMSCKSSQSLLDSG |
| | | NQKNFLTWYQQKPGQPPKLLIFWASTRESGVPD |
| | | RFTGSGSVTDFTLTISSVQAEDLAVYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 171 | DNA LC | GACATTGTGATGACCCAGTCTCCATCCTCCCTG |
| | | ACTGTGACAGCAGGAGAGAAGGTCACTATGAGC |
| | | TGCAAGTCCAGTCAGAGTCTGTTAGACAGTGGA |
| | | AATCAAAAGAACTTCTTGACCTGGTACCAGCAG |
| | | AAACCAGGGCAGCCTCCTAAACTGTTGATCTTC |
| | | TGGGCATCCACTAGGGAATCTGGGGTCCCTGAT |
| | | CGCTTCACAGGCAGTGGATCTGTAACAGATTTC |
| | | ACTCTCACCATCAGCAGTGTGCAGGCTGAAGAC |
| | | CTGGCAGTTTATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum01 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 173 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 174 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 175 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum01 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 176 | VL | EIVLTQSPATLSLSPGERATLSCKSSQSLLDSG |
| | | NQKNFLTWYQQKPGQAPRLLIYWASTRESGVPS |
| | | RFSGSGSGTEFTLTISSLQPDDFATYYCQNDYS |
| | | YPYTFGQGTKVEIK |
| SEQ ID NO: 177 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCATCA |
| | | AGGTTCAGCGGCAGTGGATCTGGGACAGAATTC |
| | | ACTCTCACCATCAGCAGCCTGCAGCCTGATGAT |
| | | TTTGCAACTTATTACTGTCAGAATGATTATAGT |
| | | TATCCGTACACGTTCGGCCAAGGGACCAAGGTG |
| | | GAAATCAAA |
| SEQ ID NO: 178 | LC | EIVLTQSPATLSLSPGERATLSCKSSQSLLDSG |
| | | NQKNFLTWYQQKPGQAPRLLIYWASTRESGVPS |
| | | RFSGSGSGTEFTLTISSLQPDDFATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 179 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCATCA |
| | | AGGTTCAGCGGCAGTGGATCTGGGACAGAATTC |
| | | ACTCTCACCATCAGCAGCCTGCAGCCTGATGAT |
| | | TTTGCAACTTATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum02 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 173 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 174 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 175 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum02 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 180 | VL | DIQMTQSPSSLSASVGDRVTITCKSSQSLLDSG |
| | | NQKNFLTWYQQKPGQAPRLLIYWASTRESGIPP |
| | | RFSGSGYGTDFTLTINNIESEDAAYYFCQNDYS |
| | | YPYTFGQGTKVEIK |
| SEQ ID NO: 181 | DNA VL | GACATCCAGATGACCCAGTCTCCATCCTCCCTG |
| | | TCTGCATCTGTAGGAGACAGAGTCACCATCACT |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGATCCCACCT |
| | | CGATTCAGTGGCAGCGGGTATGGAACAGATTTT |
| | | ACCCTCACAATTAATAACATAGAATCTGAGGAT |
| | | GCTGCATATTACTTCTGTCAGAATGATTATAGT |
| | | TATCCGTACACGTTCGGCCAAGGGACCAAGGTG |
| | | GAAATCAAA |
| SEQ ID NO: 182 | LC | DIQMTQSPSSLSASVGDRVTITCKSSQSLLDSG |
| | | NQKNFLTWYQQKPGQAPRLLIYWASTRESGIPP |
| | | RFSGSGYGTDFTLTINNIESEDAAYYFCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 183 | DNA LC | GACATCCAGATGACCCAGTCTCCATCCTCCCTG |
| | | TCTGCATCTGTAGGAGACAGAGTCACCATCACT |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGATCCCACCT |
| | | CGATTCAGTGGCAGCGGGTATGGAACAGATTTT |
| | | ACCCTCACAATTAATAACATAGAATCTGAGGAT |
| | | GCTGCATATTACTTCTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum03 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 184 | VH | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 185 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 186 | HC | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 187 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum03 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 180 | VL | DIQMTQSPSSLSASVGDRVTITCKSSQSLLDSG |
| | | NQKNFLTWYQQKPGQAPRLLIYWASTRESGIPP |
| | | RFSGSGYGTDFTLTINNIESEDAAYYFCQNDYS |
| | | YPYTFGQGTKVEIK |
| SEQ ID NO: 181 | DNA VL | GACATCCAGATGACCCAGTCTCCATCCTCCCTG |
| | | TCTGCATCTGTAGGAGACAGAGTCACCATCACT |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGATCCCACCT |
| | | CGATTCAGTGGCAGCGGGTATGGAACAGATTTT |
| | | ACCCTCACAATTAATAACATAGAATCTGAGGAT |
| | | GCTGCATATTACTTCTGTCAGAATGATTATAGT |
| | | TATCCGTACACGTTCGGCCAAGGGACCAAGGTG |
| | | GAAATCAAA |
| SEQ ID NO: 182 | LC | DIQMTQSPSSLSASVGDRVTITCKSSQSLLDSG |
| | | NQKNFLTWYQQKPGQAPRLLIYWASTRESGIPP |
| | | RFSGSGYGTDFTLTINNIESEDAAYYFCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 183 | DNA LC | GACATCCAGATGACCCAGTCTCCATCCTCCCTG |
| | | TCTGCATCTGTAGGAGACAGAGTCACCATCACT |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGATCCCACCT |
| | | CGATTCAGTGGCAGCGGGTATGGAACAGATTTT |
| | | ACCCTCACAATTAATAACATAGAATCTGAGGAT |
| | | GCTGCATATTACTTCTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum04 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 184 | VH | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 185 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 186 | HC | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 187 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum04 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 188 | VL | EIVLTQSPATLSLSPGERATLSCKSSQSLLDSG |
| | | NQKNFLTWYQQKPGKAPKLLIYWASTRESGVPS |
| | | RFSGSGSGTDFTFTISSLQPEDIATYYCQNDYS |
| | | YPYTFGQGTKVEIK |
| SEQ ID NO: 189 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTATCAGCAG |
| | | AAACCAGGGAAAGCTCCTAAGCTCCTGATCTAT |
| | | TGGGCATCCACTAGGGAATCTGGGGTCCCATCA |
| | | AGGTTCAGTGGAAGTGGATCTGGGACAGATTTT |
| | | ACTTTCACCATCAGCAGCCTGCAGCCTGAAGAT |
| | | ATTGCAACATATTACTGTCAGAATGATTATAGT |
| | | TATCCGTACACGTTCGGCCAAGGGACCAAGGTG |
| | | GAAATCAAA |
| SEQ ID NO: 190 | LC | EIVLTQSPATLSLSPGERATLSCKSSQSLLDSG |
| | | NQKNFLTWYQQKPGKAPKLLIYWASTRESGVPS |
| | | RFSGSGSGTDFTFTISSLQPEDIATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 191 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTATCAGCAG |
| | | AAACCAGGGAAAGCTCCTAAGCTCCTGATCTAT |
| | | TGGGCATCCACTAGGGAATCTGGGGTCCCATCA |
| | | AGGTTCAGTGGAAGTGGATCTGGGACAGATTTT |
| | | ACTTTCACCATCAGCAGCCTGCAGCCTGAAGAT |
| | | ATTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum05 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 173 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 174 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 175 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum05 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 188 | VL | EIVLTQSPATLSLSPGERATLSCKSSQSLLDSG |
| | | NQKNFLTWYQQKPGKAPKLLIYWASTRESGVPS |
| | | RFSGSGSGTDFTFTISSLQPEDIATYYCQNDYS |
| | | YPYTFGQGTKVEIK |
| SEQ ID NO: 189 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTATCAGCAG |
| | | AAACCAGGGAAAGCTCCTAAGCTCCTGATCTAT |
| | | TGGGCATCCACTAGGGAATCTGGGGTCCCATCA |
| | | AGGTTCAGTGGAAGTGGATCTGGGACAGATTTT |
| | | ACTTTCACCATCAGCAGCCTGCAGCCTGAAGAT |
| | | ATTGCAACATATTACTGTCAGAATGATTATAGT |
| | | TATCCGTACACGTTCGGCCAAGGGACCAAGGTG |
| | | GAAATCAAA |
| SEQ ID NO: 190 | LC | EIVLTQSPATLSLSPGERATLSCKSSQSLLDSG |
| | | NQKNFLTWYQQKPGKAPKLLIYWASTRESGVPS |
| | | RFSGSGSGTDFTFTISSLQPEDIATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 191 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTATCAGCAG |
| | | AAACCAGGGAAAGCTCCTAAGCTCCTGATCTAT |
| | | TGGGCATCCACTAGGGAATCTGGGGTCCCATCA |
| | | AGGTTCAGTGGAAGTGGATCTGGGACAGATTTT |
| | | ACTTTCACCATCAGCAGCCTGCAGCCTGAAGAT |
| | | ATTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum06 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 173 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 174 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 175 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum06 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 192 | VL | DIVMTQTPLSLPVTPGEPASISCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 193 | DNA VL | GATATTGTGATGACCCAGACTCCACTCTCCCTG |
| | | CCCGTCACCCCTGGAGAGCCGGCCTCCATCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 194 | LC | DIVMTQTPLSLPVTPGEPASISCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 195 | DNA LC | GATATTGTGATGACCCAGACTCCACTCTCCCTG |
| | | CCCGTCACCCCTGGAGAGCCGGCCTCCATCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum07 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 173 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 174 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 175 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum07 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 196 | VL | EIVLTQSPATLSLSPGERATLSCKSSQSLLDSG |
| | | NQKNFLTWYQQKPGKAPKLLIYWASTRESGVPS |
| | | RFSGSGSGTDFTFTISSLEAEDAATYYCQNDYS |
| | | YPYTFGQGTKVEIK |
| SEQ ID NO: 197 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTATCAGCAG |
| | | AAACCAGGGAAAGCTCCTAAGCTCCTGATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 198 | LC | EIVLTQSPATLSLSPGERATLSCKSSQSLLDSG |
| | | NQKNFLTWYQQKPGKAPKLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 199 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTATCAGCAG |
| | | AAACCAGGGAAAGCTCCTAAGCTCCTGATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum08 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 184 | VH | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 185 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 186 | HC | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 187 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum08 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 200 | VL | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 201 | DNA VL | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 202 | LC | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 203 | DNA LC | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum09 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 173 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 174 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 175 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum09 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 200 | VL | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 201 | DNA VL | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 202 | LC | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 203 | DNA LC | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum10 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 184 | VH | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 185 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 186 | HC | EVQLVQSGAEVKKPGESLRISCKGSGYTFTTYW |
| | | MHWIRQSPSRGLEWLGNIYPGTGGSNFDEFFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHEPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYECKVSNEGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGE |
| SEQ ID NO: 187 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum10 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 204 | VL | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 205 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 206 | LC | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 207 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum11 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 173 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 174 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 175 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum11 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 204 | VL | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 205 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 206 | LC | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 207 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum12 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 173 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 174 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 175 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum12 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 208 | VL | DIQMTQSPSSLSASVGDRVTITCKSSQSLLDSG |
| | | NQKNFLTWYLQKPGQSPQLLIYWASTRESGVPS |
| | | RFSGSGSGTDFTFTISSLEAEDAATYYCQNDYS |
| | | YPYTFGQGTKVEIK |
| SEQ ID NO: 209 | DNA VL | GACATCCAGATGACCCAGTCTCCATCCTCCCTG |
| | | TCTGCATCTGTAGGAGACAGAGTCACCATCACT |
| | | TGCAAGTCCAGTCAGAGTCTGTTAGACAGTGGA |
| | | AATCAAAAGAACTTCTTGACCTGGTACCTGCAG |
| | | AAGCCAGGGCAGTCTCCACAGCTCCTGATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 210 | LC | DIQMTQSPSSLSASVGDRVTITCKSSQSLLDSG |
| | | NQKNFLTWYLQKPGQSPQLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 211 | DNA LC | GACATCCAGATGACCCAGTCTCCATCCTCCCTG |
| | | TCTGCATCTGTAGGAGACAGAGTCACCATCACT |
| | | TGCAAGTCCAGTCAGAGTCTGTTAGACAGTGGA |
| | | AATCAAAAGAACTTCTTGACCTGGTACCTGCAG |
| | | AAGCCAGGGCAGTCTCCACAGCTCCTGATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum13 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 173 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 174 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 175 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCACTGGACAAGGG |
| | | CTTGAGTGGATGGGTAATATTTATCCTGGTACT |
| | | GGTGGTTCTAACTTCGATGAGAAGTTCAAGAAC |
| | | AGAGTCACGATTACCGCGGACAAATCCACGAGC |
| | | ACAGCCTACATGGAGCTGAGCAGCCTGAGATCT |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum13 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 212 | VL | DVVMTQSPLSLPVTLGQPASISCKSSQSLLDSG |
| | | NQKNFLTWYQQKPGKAPKLLIYWASTRESGVPS |
| | | RFSGSGSGTDFTFTISSLEAEDAATYYCQNDYS |
| | | YPYTFGQGTKVEIK |
| SEQ ID NO: 213 | DNA VL | GATGTTGTGATGACTCAGTCTCCACTCTCCCTG |
| | | CCCGTCACCCTTGGACAGCCGGCCTCCATCTCC |
| | | TGCAAGTCCAGTCAGAGTCTGTTAGACAGTGGA |
| | | AATCAAAAGAACTTCTTAACCTGGTATCAGCAG |
| | | AAACCAGGGAAAGCTCCTAAGCTCCTGATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 214 | LC | DVVMTQSPLSLPVTLGQPASISCKSSQSLLDSG |
| | | NQKNFLTWYQQKPGKAPKLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 215 | DNA LC | GATGTTGTGATGACTCAGTCTCCACTCTCCCTG |
| | | CCCGTCACCCTTGGACAGCCGGCCTCCATCTCC |
| | | TGCAAGTCCAGTCAGAGTCTGTTAGACAGTGGA |
| | | AATCAAAAGAACTTCTTAACCTGGTATCAGCAG |
| | | AAACCAGGGAAAGCTCCTAAGCTCCTGATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum14 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 216 | VH | QVQLVQSGAEVKKPGASVKVSCKASGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 217 | DNA VH | CAGGTTCAGCTGGTGCAGTCTGGAGCTGAGGTG |
| | | AAGAAGCCTGGGGCCTCAGTGAAGGTCTCCTGC |
| | | AAGGCTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTACTGGGGCCAGGGC |
| | | ACCACCGTGACCGTGTCCTCC |
| SEQ ID NO: 218 | HC | QVQLVQSGAEVKKPGASVKVSCKASGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 219 | DNA HC | CAGGTTCAGCTGGTGCAGTCTGGAGCTGAGGTG |
| | | AAGAAGCCTGGGGCCTCAGTGAAGGTCTCCTGC |
| | | AAGGCTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTACTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum14 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 204 | VL | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 205 | DNA VL | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 206 | LC | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 207 | DNA LC | GAAATTGTGTTGACACAGTCTCCAGCCACCCTG |
| | | TCTTTGTCTCCAGGGGAAAGAGCCACCCTCTCC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum15 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 216 | VH | QVQLVQSGAEVKKPGASVKVSCKASGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 217 | DNA VH | CAGGTTCAGCTGGTGCAGTCTGGAGCTGAGGTG |
| | | AAGAAGCCTGGGGCCTCAGTGAAGGTCTCCTGC |
| | | AAGGCTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTACTGGGGCCAGGGC |
| | | ACCACCGTGACCGTGTCCTCC |
| SEQ ID NO: 218 | HC | QVQLVQSGAEVKKPGASVKVSCKASGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 219 | DNA HC | CAGGTTCAGCTGGTGCAGTCTGGAGCTGAGGTG |
| | | AAGAAGCCTGGGGCCTCAGTGAAGGTCTCCTGC |
| | | AAGGCTTCTGGCTACACATTCACCACTTACTGG |
| | | ATGCACTGGATCAGGCAGTCCCCATCGAGAGGC |
| | | CTTGAGTGGCTGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTACTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum15 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 200 | VL | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 201 | DNA VL | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 202 | LC | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 203 | DNA LC | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-hum16 HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 220 | VH | EVQLVQSGAEVKKPGESLRISCKGSGYTFTTYW |
| | | MHWVRQAPGQGLEWMGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 221 | DNA VH | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCCCTGGACAAGGG |
| | | CTTGAGTGGATGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCC |
| SEQ ID NO: 222 | HC | EVQLVQSGAEVKKPGESLRISCKGSGYTFTTYW |
| | | MHWVRQAPGQGLEWMGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLGK |
| SEQ ID NO: 223 | DNA HC | GAAGTGCAGCTGGTGCAGTCTGGAGCAGAGGTG |
| | | AAAAAGCCCGGGGAGTCTCTGAGGATCTCCTGT |
| | | AAGGGTTCTGGCTACACATTCACCACTTACTGG |
| | | ATGCACTGGGTGCGACAGGCCCCTGGACAAGGG |
| | | CTTGAGTGGATGGGTAATATTTATCCTGGTACT |
| | | GGTGGTTCTAACTTCGATGAGAAGTTCAAGAAC |
| | | AGATTCACCATCTCCAGAGACAATTCCAAGAAC |
| | | ACGCTGTATCTTCAAATGAACAGCCTGAGAGCC |
| | | GAGGACACGGCCGTGTATTACTGTACAAGATGG |
| | | ACTACTGGGACGGGAGCTTATTGGGGCCAGGGC |
| | | ACCACCGTGACCGTGTCCTCCGCTTCCACCAAG |
| | | GGCCCATCCGTCTTCCCCCTGGCGCCCTGCTCC |
| | | AGGAGCACCTCCGAGAGCACAGCCGCCCTGGGC |
| | | TGCCTGGTCAAGGACTACTTCCCCGAACCGGTG |
| | | ACGGTGTCGTGGAACTCAGGCGCCCTGACCAGC |
| | | GGCGTGCACACCTTCCCGGCTGTCCTACAGTCC |
| | | TCAGGACTCTACTCCCTCAGCAGCGTGGTGACC |
| | | GTGCCCTCCAGCAGCTTGGGCACGAAGACCTAC |
| | | ACCTGCAACGTAGATCACAAGCCCAGCAACACC |
| | | AAGGTGGACAAGAGAGTTGAGTCCAAATATGGT |
| | | CCCCCATGCCCACCGTGCCCAGCACCTGAGTTC |
| | | CTGGGGGGACCATCAGTCTTCCTGTTCCCCCCA |
| | | AAACCCAAGGACACTCTCATGATCTCCCGGACC |
| | | CCTGAGGTCACGTGCGTGGTGGTGGACGTGAGC |
| | | CAGGAAGACCCCGAGGTCCAGTTCAACTGGTAC |
| | | GTGGATGGCGTGGAGGTGCATAATGCCAAGACA |
| | | AAGCCGCGGGAGGAGCAGTTCAACAGCACGTAC |
| | | CGTGTGGTCAGCGTCCTCACCGTCCTGCACCAG |
| | | GACTGGCTGAACGGCAAGGAGTACAAGTGCAAG |
| | | GTGTCCAACAAAGGCCTCCCGTCCTCCATCGAG |
| | | AAAACCATCTCCAAAGCCAAAGGGCAGCCCCGA |
| | | GAGCCACAGGTGTACACCCTGCCCCCATCCCAG |
| | | GAGGAGATGACCAAGAACCAGGTCAGCCTGACC |
| | | TGCCTGGTCAAAGGCTTCTACCCCAGCGACATC |
| | | GCCGTGGAGTGGGAGAGCAATGGGCAGCCGGAG |
| | | AACAACTACAAGACCACGCCTCCCGTGCTGGAC |
| | | TCCGACGGCTCCTTCTTCCTCTACAGCAGGCTA |
| | | ACCGTGGACAAGAGCAGGTGGCAGGAGGGGAAT |
| | | GTCTTCTCATGCTCCGTGATGCATGAGGCTCTG |
| | | CACAACCACTACACACAGAAGAGCCTCTCCCTG |
| | | TCTCTGGGTAAA |

BAP049-hum16 LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 200 | VL | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 201 | DNA VL | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAA |
| SEQ ID NO: 202 | LC | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 203 | DNA LC | GAAATTGTGCTGACTCAGTCTCCAGACTTTCAG |
| | | TCTGTGACTCCAAAGGAGAAAGTCACCATCACC |
| | | TGCAAGTCCAGTCAGAGTCTGTTAGACAGTGGA |
| | | AATCAAAAGAACTTCTTGACCTGGTACCAGCAG |
| | | AAACCTGGCCAGGCTCCCAGGCTCCTCATCTAT |
| | | TGGGCATCCACTAGGGAATCTGGGGTCCCCTCG |
| | | AGGTTCAGTGGCAGTGGATCTGGGACAGATTTC |
| | | ACCTTTACCATCAGTAGCCTGGAAGCTGAAGAT |
| | | GCTGCAACATATTACTGTCAGAATGATTATAGT |
| | | TATCCGTACACGTTCGGCCAAGGGACCAAGGTG |
| | | GAAATCAAACGTACGGTGGCTGCACCATCTGTC |
| | | TTCATCTTCCCGCCATCTGATGAGCAGTTGAAA |
| | | TCTGGAACTGCCTCTGTTGTGTGCCTGCTGAAT |
| | | AACTTCTATCCCAGAGAGGCCAAAGTACAGTGG |
| | | AAGGTGGATAACGCCCTCCAATCGGGTAACTCC |
| | | CAGGAGAGTGTCACAGAGCAGGACAGCAAGGAC |
| | | AGCACCTACAGCCTCAGCAGCACCCTGACGCTG |
| | | AGCAAAGCAGACTACGAGAAACACAAAGTCTAC |
| | | GCCTGCGAAGTCACCCATCAGGGCCTGAGCTCG |
| | | CCCGTCACAAAGAGCTTCAACAGGGGAGAGTGT |

BAP049-Clone-A HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 224 | DNA VH | GAAGTGCAGCTGGTGCAGTCTGGCGCCGAAGTG |
| | | AAGAAGCCTGGCGAGTCCCTGCGGATCTCCTGC |
| | | AAGGGCTCTGGCTACACCTTCACCACCTACTGG |
| | | ATGCACTGGGTGCGACAGGCTACCGGCCAGGGC |
| | | CTGGAATGGATGGGCAACATCTATCCTGGCACC |
| | | GGCGGCTCCAACTTCGACGAGAAGTTCAAGAAC |
| | | AGAGTGACCATCACCGCCGACAAGTCCACCTCC |
| | | ACCGCCTACATGGAACTGTCCTCCCTGAGATCC |
| | | GAGGACACCGCCGTGTACTACTGCACCCGGTGG |
| | | ACAACCGGCACAGGCGCTTATTGGGGCCAGGGC |
| | | ACCACAGTGACCGTGTCCTCT |
| SEQ ID NO: 225 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLG |
| SEQ ID NO: 226 | DNA HC | GAAGTGCAGCTGGTGCAGTCTGGCGCCGAAGTG |
| | | AAGAAGCCTGGCGAGTCCCTGCGGATCTCCTGC |
| | | AAGGGCTCTGGCTACACCTTCACCACCTACTGG |
| | | ATGCACTGGGTGCGACAGGCTACCGGCCAGGGC |
| | | CTGGAATGGATGGGCAACATCTATCCTGGCACC |
| | | GGCGGCTCCAACTTCGACGAGAAGTTCAAGAAC |
| | | AGAGTGACCATCACCGCCGACAAGTCCACCTCC |
| | | ACCGCCTACATGGAACTGTCCTCCCTGAGATCC |
| | | GAGGACACCGCCGTGTACTACTGCACCCGGTGG |
| | | ACAACCGGCACAGGCGCTTATTGGGGCCAGGGC |
| | | ACCACAGTGACCGTGTCCTCTGCTTCTACCAAG |
| | | GGGCCCAGCGTGTTCCCCCTGGCCCCCTGCTCC |
| | | AGAAGCACCAGCGAGAGCACAGCCGCCCTGGGC |
| | | TGCCTGGTGAAGGACTACTTCCCCGAGCCCGTG |
| | | ACCGTGTCCTGGAACAGCGGAGCCCTGACCAGC |
| | | GGCGTGCACACCTTCCCCGCCGTGCTGCAGAGC |
| | | AGCGGCCTGTACAGCCTGAGCAGCGTGGTGACC |
| | | GTGCCCAGCAGCAGCCTGGGCACCAAGACCTAC |
| | | ACCTGTAACGTGGACCACAAGCCCAGCAACACC |
| | | AAGGTGGACAAGAGGGTGGAGAGCAAGTACGGC |
| | | CCACCCTGCCCCCCCTGCCCAGCCCCCGAGTTC |
| | | CTGGGCGGACCCAGCGTGTTCCTGTTCCCCCCC |
| | | AAGCCCAAGGACACCCTGATGATCAGCAGAACC |
| | | CCCGAGGTGACCTGTGTGGTGGTGGACGTGTCC |
| | | CAGGAGGACCCCGAGGTCCAGTTCAACTGGTAC |
| | | GTGGACGGCGTGGAGGTGCACAACGCCAAGACC |
| | | AAGCCCAGAGAGGAGCAGTTTAACAGCACCTAC |
| | | CGGGTGGTGTCCGTGCTGACCGTGCTGCACCAG |
| | | GACTGGCTGAACGGCAAAGAGTACAAGTGTAAG |
| | | GTCTCCAACAAGGGCCTGCCAAGCAGCATCGAA |
| | | AAGACCATCAGCAAGGCCAAGGGCCAGCCTAGA |
| | | GAGCCCCAGGTCTACACCCTGCCACCCAGCCAA |
| | | GAGGAGATGACCAAGAACCAGGTGTCCCTGACC |
| | | TGTCTGGTGAAGGGCTTCTACCCAAGCGACATC |
| | | GCCGTGGAGTGGGAGAGCAACGGCCAGCCCGAG |
| | | AACAACTACAAGACCACCCCCCCAGTGCTGGAC |
| | | AGCGACGGCAGCTTCTTCCTGTACAGCAGGCTG |
| | | ACCGTGGACAAGTCCAGATGGCAGGAGGGCAAC |
| | | GTCTTTAGCTGCTCCGTGATGCACGAGGCCCTG |
| | | CACAACCACTACACCCAGAAGAGCCTGAGCCTG |
| | | TCCCTGGGC |

BAP049-Clone-A LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 176 | VL | EIVLTQSPATLSLSPGERATLSCKSSQSLLDSG |
| | | NQKNFLTWYQQKPGQAPRLLIYWASTRESGVPS |
| | | RFSGSGSGTEFTLTISSLQPDDFATYYCQNDYS |
| | | YPYTFGQGTKVEIK |
| SEQ ID NO: 227 | DNA VL | GAGATCGTGCTGACCCAGTCCCCTGCCACCCTG |
| | | TCACTGTCTCCAGGCGAGAGAGCTACCCTGTCC |
| | | TGCAAGTCCTCCCAGTCCCTGCTGGACTCCGGC |
| | | AACCAGAAGAACTTCCTGACCTGGTATCAGCAG |
| | | AAGCCCGGCCAGGCCCCCAGACTGCTGATCTAC |
| | | TGGGCCTCCACCCGGGAATCTGGCGTGCCCTCT |
| | | AGATTCTCCGGCTCCGGCTCTGGCACCGAGTTT |
| | | ACCCTGACCATCTCCAGCCTGCAGCCCGACGAC |
| | | TTCGCCACCTACTACTGCCAGAACGACTACTCC |
| | | TACCCCTACACCTTCGGCCAGGGCACCAAGGTG |
| | | GAAATCAAG |
| SEQ ID NO: 178 | LC | EIVLTQSPATLSLSPGERATLSCKSSQSLLDSG |
| | | NQKNFLTWYQQKPGQAPRLLIYWASTRESGVPS |
| | | RFSGSGSGTEFTLTISSLQPDDFATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 228 | DNA LC | GAGATCGTGCTGACCCAGTCCCCTGCCACCCTG |
| | | TCACTGTCTCCAGGCGAGAGAGCTACCCTGTCC |
| | | TGCAAGTCCTCCCAGTCCCTGCTGGACTCCGGC |
| | | AACCAGAAGAACTTCCTGACCTGGTATCAGCAG |
| | | AAGCCCGGCCAGGCCCCCAGACTGCTGATCTAC |
| | | TGGGCCTCCACCCGGGAATCTGGCGTGCCCTCT |
| | | AGATTCTCCGGCTCCGGCTCTGGCACCGAGTTT |
| | | ACCCTGACCATCTCCAGCCTGCAGCCCGACGAC |
| | | TTCGCCACCTACTACTGCCAGAACGACTACTCC |
| | | TACCCCTACACCTTCGGCCAGGGCACCAAGGTG |
| | | GAAATCAAGCGTACGGTGGCCGCTCCCAGCGTG |
| | | TTCATCTTCCCCCCAAGCGACGAGCAGCTGAAG |
| | | AGCGGCACCGCCAGCGTGGTGTGTCTGCTGAAC |
| | | AACTTCTACCCCAGGGAGGCCAAGGTGCAGTGG |
| | | AAGGTGGACAACGCCCTGCAGAGCGGCAACAGC |
| | | CAGGAGAGCGTCACCGAGCAGGACAGCAAGGAC |
| | | TCCACCTACAGCCTGAGCAGCACCCTGACCCTG |
| | | AGCAAGGCCGACTACGAGAAGCACAAGGTGTAC |
| | | GCCTGTGAGGTGACCCACCAGGGCCTGTCCAGC |
| | | CCCGTGACCAAGAGCTTCAACAGGGGCGAGTGC |

BAP049-Clone-B HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 229 | DNA VH | GAGGTGCAGCTGGTGCAGTCAGGCGCCGAAGTG |
| | | AAGAAGCCCGGCGAGTCACTGAGAATTAGCTGT |
| | | AAAGGTTCAGGCTACACCTTCACTACCTACTGG |
| | | ATGCACTGGGTCCGCCAGGCTACCGGTCAAGGC |
| | | CTCGAGTGGATGGGTAATATCTACCCCGGCACC |
| | | GGCGGCTCTAACTTCGACGAGAAGTTTAAGAAT |
| | | AGAGTGACTATCACCGCCGATAAGTCTACTAGC |
| | | ACCGCCTATATGGAACTGTCTAGCCTGAGATCA |
| | | GAGGACACCGCCGTCTACTACTGCACTAGGTGG |
| | | ACTACCGGCACAGGCGCCTACTGGGGTCAAGGC |
| | | ACTACCGTGACCGTGTCTAGC |
| SEQ ID NO: 225 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLG |
| SEQ ID NO: 230 | DNA HC | GAGGTGCAGCTGGTGCAGTCAGGCGCCGAAGTG |
| | | AAGAAGCCCGGCGAGTCACTGAGAATTAGCTGT |
| | | AAAGGTTCAGGCTACACCTTCACTACCTACTGG |
| | | ATGCACTGGGTCCGCCAGGCTACCGGTCAAGGC |
| | | CTCGAGTGGATGGGTAATATCTACCCCGGCACC |
| | | GGCGGCTCTAACTTCGACGAGAAGTTTAAGAAT |
| | | AGAGTGACTATCACCGCCGATAAGTCTACTAGC |
| | | ACCGCCTATATGGAACTGTCTAGCCTGAGATCA |
| | | GAGGACACCGCCGTCTACTACTGCACTAGGTGG |
| | | ACTACCGGCACAGGCGCCTACTGGGGTCAAGGC |
| | | ACTACCGTGACCGTGTCTAGCGCTAGCACTAAG |
| | | GGCCCGTCCGTGTTCCCCCTGGCACCTTGTAGC |
| | | CGGAGCACTAGCGAATCCACCGCTGCCCTCGGC |
| | | TGCCTGGTCAAGGATTACTTCCCGGAGCCCGTG |
| | | ACCGTGTCCTGGAACAGCGGAGCCCTGACCTCC |
| | | GGAGTGCACACCTTCCCCGCTGTGCTGCAGAGC |
| | | TCCGGGCTGTACTCGCTGTCGTCGGTGGTCACG |
| | | GTGCCTTCATCTAGCCTGGGTACCAAGACCTAC |
| | | ACTTGCAACGTGGACCACAAGCCTTCCAACACT |
| | | AAGGTGGACAAGCGCGTCGAATCGAAGTACGGC |
| | | CCACCGTGCCCGCCTTGTCCCGCGCCGGAGTTC |
| | | CTCGGCGGTCCCTCGGTCTTTCTGTTCCCACCG |
| | | AAGCCCAAGGACACTTTGATGATTTCCCGCACC |
| | | CCTGAAGTGACATGCGTGGTCGTGGACGTGTCA |
| | | CAGGAAGATCCGGAGGTGCAGTTCAATTGGTAC |
| | | GTGGATGGCGTCGAGGTGCACAACGCCAAAACC |
| | | AAGCCGAGGGAGGAGCAGTTCAACTCCACTTAC |
| | | CGCGTCGTGTCCGTGCTGACGGTGCTGCATCAG |
| | | GACTGGCTGAACGGGAAGGAGTACAAGTGCAAA |
| | | GTGTCCAACAAGGGACTTCCTAGCTCAATCGAA |
| | | AAGACCATCTCGAAAGCCAAGGGACAGCCCCGG |
| | | GAACCCCAAGTGTATACCCTGCCACCGAGCCAG |
| | | GAAGAAATGACTAAGAACCAAGTCTCATTGACT |
| | | TGCCTTGTGAAGGGCTTCTACCCATCGGATATC |
| | | GCCGTGGAATGGGAGTCCAACGGCCAGCCGGAA |
| | | AACAACTACAAGACCACCCCTCCGGTGCTGGAC |
| | | TCAGACGGATCCTTCTTCCTCTACTCGCGGCTG |
| | | ACCGTGGATAAGAGCAGATGGCAGGAGGGAAAT |
| | | GTGTTCAGCTGTTCTGTGATGCATGAAGCCCTG |
| | | CACAACCACTACACTCAGAAGTCCCTGTCCCTC |
| | | TCCCTGGGA |

BAP049-Clone-B LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 188 | VL | EIVLTQSPATLSLSPGERATLSCKSSQSLLDSG |
| | | NQKNFLTWYQQKPGKAPKLLIYWASTRESGVPS |
| | | RFSGSGSGTDFTFTISSLQPEDIATYYCQNDYS |
| | | YPYTFGQGTKVEIK |
| SEQ ID NO: 231 | DNA VL | GAGATCGTCCTGACTCAGTCACCCGCTACCCTG |
| | | AGCCTGAGCCCTGGCGAGCGGGCTACACTGAGC |
| | | TGTAAATCTAGTCAGTCACTGCTGGATAGCGGT |
| | | AATCAGAAGAACTTCCTGACCTGGTATCAGCAG |
| | | AAGCCCGGTAAAGCCCCTAAGCTGCTGATCTAC |
| | | TGGGCCTCTACTAGAGAATCAGGCGTGCCCTCT |
| | | AGGTTTAGCGGTAGCGGTAGTGGCACCGACTTC |
| | | ACCTTCACTATCTCTAGCCTGCAGCCCGAGGAT |
| | | ATCGCTACCTACTACTGTCAGAACGACTATAGC |
| | | TACCCCTACACCTTCGGTCAAGGCACTAAGGTC |
| | | GAGATTAAG |
| SEQ ID NO: 190 | LC | EIVLTQSPATLSLSPGERATLSCKSSQSLLDSG |
| | | NQKNFLTWYQQKPGKAPKLLIYWASTRESGVPS |
| | | RFSGSGSGTDFTFTISSLQPEDIATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 232 | DNA LC | GAGATCGTCCTGACTCAGTCACCCGCTACCCTG |
| | | AGCCTGAGCCCTGGCGAGCGGGCTACACTGAGC |
| | | TGTAAATCTAGTCAGTCACTGCTGGATAGCGGT |
| | | AATCAGAAGAACTTCCTGACCTGGTATCAGCAG |
| | | AAGCCCGGTAAAGCCCCTAAGCTGCTGATCTAC |
| | | TGGGCCTCTACTAGAGAATCAGGCGTGCCCTCT |
| | | AGGTTTAGCGGTAGCGGTAGTGGCACCGACTTC |
| | | ACCTTCACTATCTCTAGCCTGCAGCCCGAGGAT |
| | | ATCGCTACCTACTACTGTCAGAACGACTATAGC |
| | | TACCCCTACACCTTCGGTCAAGGCACTAAGGTC |
| | | GAGATTAAGCGTACGGTGGCCGCTCCCAGCGTG |
| | | TTCATCTTCCCCCCCAGCGACGAGCAGCTGAAG |
| | | AGCGGCACCGCCAGCGTGGTGTGCCTGCTGAAC |
| | | AACTTCTACCCCCGGGAGGCCAAGGTGCAGTGG |
| | | AAGGTGGACAACGCCCTGCAGAGCGGCAACAGC |
| | | CAGGAGAGCGTCACCGAGCAGGACAGCAAGGAC |
| | | TCCACCTACAGCCTGAGCAGCACCCTGACCCTG |
| | | AGCAAGGCCGACTACGAGAAGCATAAGGTGTAC |
| | | GCCTGCGAGGTGACCCACCAGGGCCTGTCCAGC |
| | | CCCGTGACCAAGAGCTTCAACAGGGGCGAGTGC |

BAP049-Clone-C HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 224 | DNA VH | GAAGTGCAGCTGGTGCAGTCTGGCGCCGAAGTG |
| | | AAGAAGCCTGGCGAGTCCCTGCGGATCTCCTGC |
| | | AAGGGCTCTGGCTACACCTTCACCACCTACTGG |
| | | ATGCACTGGGTGCGACAGGCTACCGGCCAGGGC |
| | | CTGGAATGGATGGGCAACATCTATCCTGGCACC |
| | | GGCGGCTCCAACTTCGACGAGAAGTTCAAGAAC |
| | | AGAGTGACCATCACCGCCGACAAGTCCACCTCC |
| | | ACCGCCTACATGGAACTGTCCTCCCTGAGATCC |
| | | GAGGACACCGCCGTGTACTACTGCACCCGGTGG |
| | | ACAACCGGCACAGGCGCTTATTGGGGCCAGGGC |
| | | ACCACAGTGACCGTGTCCTCT |
| SEQ ID NO: 225 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLG |
| SEQ ID NO: 226 | DNA HC | GAAGTGCAGCTGGTGCAGTCTGGCGCCGAAGTG |
| | | AAGAAGCCTGGCGAGTCCCTGCGGATCTCCTGC |
| | | AAGGGCTCTGGCTACACCTTCACCACCTACTGG |
| | | ATGCACTGGGTGCGACAGGCTACCGGCCAGGGC |
| | | CTGGAATGGATGGGCAACATCTATCCTGGCACC |
| | | GGCGGCTCCAACTTCGACGAGAAGTTCAAGAAC |
| | | AGAGTGACCATCACCGCCGACAAGTCCACCTCC |
| | | ACCGCCTACATGGAACTGTCCTCCCTGAGATCC |
| | | GAGGACACCGCCGTGTACTACTGCACCCGGTGG |
| | | ACAACCGGCACAGGCGCTTATTGGGGCCAGGGC |
| | | ACCACAGTGACCGTGTCCTCTGCTTCTACCAAG |
| | | GGGCCCAGCGTGTTCCCCCTGGCCCCCTGCTCC |
| | | AGAAGCACCAGCGAGAGCACAGCCGCCCTGGGC |
| | | TGCCTGGTGAAGGACTACTTCCCCGAGCCCGTG |
| | | ACCGTGTCCTGGAACAGCGGAGCCCTGACCAGC |
| | | GGCGTGCACACCTTCCCCGCCGTGCTGCAGAGC |
| | | AGCGGCCTGTACAGCCTGAGCAGCGTGGTGACC |
| | | GTGCCCAGCAGCAGCCTGGGCACCAAGACCTAC |
| | | ACCTGTAACGTGGACCACAAGCCCAGCAACACC |
| | | AAGGTGGACAAGAGGGTGGAGAGCAAGTACGGC |
| | | CCACCCTGCCCCCCCTGCCCAGCCCCCGAGTTC |
| | | CTGGGCGGACCCAGCGTGTTCCTGTTCCCCCCC |
| | | AAGCCCAAGGACACCCTGATGATCAGCAGAACC |
| | | CCCGAGGTGACCTGTGTGGTGGTGGACGTGTCC |
| | | CAGGAGGACCCCGAGGTCCAGTTCAACTGGTAC |
| | | GTGGACGGCGTGGAGGTGCACAACGCCAAGACC |
| | | AAGCCCAGAGAGGAGCAGTTTAACAGCACCTAC |
| | | CGGGTGGTGTCCGTGCTGACCGTGCTGCACCAG |
| | | GACTGGCTGAACGGCAAAGAGTACAAGTGTAAG |
| | | GTCTCCAACAAGGGCCTGCCAAGCAGCATCGAA |
| | | AAGACCATCAGCAAGGCCAAGGGCCAGCCTAGA |
| | | GAGCCCCAGGTCTACACCCTGCCACCCAGCCAA |
| | | GAGGAGATGACCAAGAACCAGGTGTCCCTGACC |
| | | TGTCTGGTGAAGGGCTTCTACCCAAGCGACATC |
| | | GCCGTGGAGTGGGAGAGCAACGGCCAGCCCGAG |
| | | AACAACTACAAGACCACCCCCCCAGTGCTGGAC |
| | | AGCGACGGCAGCTTCTTCCTGTACAGCAGGCTG |
| | | ACCGTGGACAAGTCCAGATGGCAGGAGGGCAAC |
| | | GTCTTTAGCTGCTCCGTGATGCACGAGGCCCTG |
| | | CACAACCACTACACCCAGAAGAGCCTGAGCCTG |
| | | TCCCTGGGC |

BAP049-Clone-C LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 200 | VL | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 233 | DNA VL | GAGATCGTGCTGACCCAGTCCCCCGACTTCCAG |
| | | TCCGTGACCCCCAAAGAAAAAGTGACCATCACA |
| | | TGCAAGTCCTCCCAGTCCCTGCTGGACTCCGGC |
| | | AACCAGAAGAACTTCCTGACCTGGTATCAGCAG |
| | | AAGCCCGGCCAGGCCCCCAGACTGCTGATCTAC |
| | | TGGGCCTCCACCCGGGAATCTGGCGTGCCCTCT |
| | | AGATTCTCCGGCTCCGGCTCTGGCACCGACTTT |
| | | ACCTTCACCATCTCCAGCCTGGAAGCCGAGGAC |
| | | GCCGCCACCTACTACTGCCAGAACGACTACTCC |
| | | TACCCCTACACCTTCGGCCAGGGCACCAAGGTG |
| | | GAAATCAAG |
| SEQ ID NO: 202 | LC | EIVLTQSPDFQSVTPKEKVTITCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 234 | DNA LC | GAGATCGTGCTGACCCAGTCCCCCGACTTCCAG |
| | | TCCGTGACCCCCAAAGAAAAAGTGACCATCACA |
| | | TGCAAGTCCTCCCAGTCCCTGCTGGACTCCGGC |
| | | AACCAGAAGAACTTCCTGACCTGGTATCAGCAG |
| | | AAGCCCGGCCAGGCCCCCAGACTGCTGATCTAC |
| | | TGGGCCTCCACCCGGGAATCTGGCGTGCCCTCT |
| | | AGATTCTCCGGCTCCGGCTCTGGCACCGACTTT |
| | | ACCTTCACCATCTCCAGCCTGGAAGCCGAGGAC |
| | | GCCGCCACCTACTACTGCCAGAACGACTACTCC |
| | | TACCCCTACACCTTCGGCCAGGGCACCAAGGTG |
| | | GAAATCAAGCGTACGGTGGCCGCTCCCAGCGTG |
| | | TTCATCTTCCCCCCAAGCGACGAGCAGCTGAAG |
| | | AGCGGCACCGCCAGCGTGGTGTGTCTGCTGAAC |
| | | AACTTCTACCCCAGGGAGGCCAAGGTGCAGTGG |
| | | AAGGTGGACAACGCCCTGCAGAGCGGCAACAGC |
| | | CAGGAGAGCGTCACCGAGCAGGACAGCAAGGAC |
| | | TCCACCTACAGCCTGAGCAGCACCCTGACCCTG |
| | | AGCAAGGCCGACTACGAGAAGCACAAGGTGTAC |
| | | GCCTGTGAGGTGACCCACCAGGGCCTGTCCAGC |
| | | CCCGTGACCAAGAGCTTCAACAGGGGCGAGTGC |

BAP049-Clone-D HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 184 | VH | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSS |
| SEQ ID NO: 235 | DNA VH | GAAGTGCAGCTGGTGCAGTCTGGCGCCGAAGTG |
| | | AAGAAGCCTGGCGAGTCCCTGCGGATCTCCTGC |
| | | AAGGGCTCTGGCTACACCTTCACCACCTACTGG |
| | | ATGCACTGGATCCGGCAGTCCCCCTCTAGGGGC |
| | | CTGGAATGGCTGGGCAACATCTACCCTGGCACC |
| | | GGCGGCTCCAACTTCGACGAGAAGTTCAAGAAC |
| | | AGGTTCACCATCTCCCGGGACAACTCCAAGAAC |
| | | ACCCTGTACCTGCAGATGAACTCCCTGCGGGCC |
| | | GAGGACACCGCCGTGTACTACTGTACCAGATGG |
| | | ACCACCGGAACCGGCGCCTATTGGGGCCAGGGC |
| | | ACAACAGTGACCGTGTCCTCC |
| SEQ ID NO: 236 | HC | EVQLVQSGAEVKKPGESLRISCKGSGYTFTTYW |
| | | MHWIRQSPSRGLEWLGNIYPGTGGSNFDEKFKN |
| | | RFTISRDNSKNTLYLQMNSLRAEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLG |
| SEQ ID NO: 237 | DNA HC | GAAGTGCAGCTGGTGCAGTCTGGCGCCGAAGTG |
| | | AAGAAGCCTGGCGAGTCCCTGCGGATCTCCTGC |
| | | AAGGGCTCTGGCTACACCTTCACCACCTACTGG |
| | | ATGCACTGGATCCGGCAGTCCCCCTCTAGGGGC |
| | | CTGGAATGGCTGGGCAACATCTACCCTGGCACC |
| | | GGCGGCTCCAACTTCGACGAGAAGTTCAAGAAC |
| | | AGGTTCACCATCTCCCGGGACAACTCCAAGAAC |
| | | ACCCTGTACCTGCAGATGAACTCCCTGCGGGCC |
| | | GAGGACACCGCCGTGTACTACTGTACCAGATGG |
| | | ACCACCGGAACCGGCGCCTATTGGGGCCAGGGC |
| | | ACAACAGTGACCGTGTCCTCCGCTTCTACCAAG |
| | | GGGCCCAGCGTGTTCCCCCTGGCCCCCTGCTCC |
| | | AGAAGCACCAGCGAGAGCACAGCCGCCCTGGGC |
| | | TGCCTGGTGAAGGACTACTTCCCCGAGCCCGTG |
| | | ACCGTGTCCTGGAACAGCGGAGCCCTGACCAGC |
| | | GGCGTGCACACCTTCCCCGCCGTGCTGCAGAGC |
| | | AGCGGCCTGTACAGCCTGAGCAGCGTGGTGACC |
| | | GTGCCCAGCAGCAGCCTGGGCACCAAGACCTAC |
| | | ACCTGTAACGTGGACCACAAGCCCAGCAACACC |
| | | AAGGTGGACAAGAGGGTGGAGAGCAAGTACGGC |
| | | CCACCCTGCCCCCCCTGCCCAGCCCCCGAGTTC |
| | | CTGGGCGGACCCAGCGTGTTCCTGTTCCCCCCC |
| | | AAGCCCAAGGACACCCTGATGATCAGCAGAACC |
| | | CCCGAGGTGACCTGTGTGGTGGTGGACGTGTCC |
| | | CAGGAGGACCCCGAGGTCCAGTTCAACTGGTAC |
| | | GTGGACGGCGTGGAGGTGCACAACGCCAAGACC |
| | | AAGCCCAGAGAGGAGCAGTTTAACAGCACCTAC |
| | | CGGGTGGTGTCCGTGCTGACCGTGCTGCACCAG |
| | | GACTGGCTGAACGGCAAAGAGTACAAGTGTAAG |
| | | GTCTCCAACAAGGGCCTGCCAAGCAGCATCGAA |
| | | AAGACCATCAGCAAGGCCAAGGGCCAGCCTAGA |
| | | GAGCCCCAGGTCTACACCCTGCCACCCAGCCAA |
| | | GAGGAGATGACCAAGAACCAGGTGTCCCTGACC |
| | | TGTCTGGTGAAGGGCTTCTACCCAAGCGACATC |
| | | GCCGTGGAGTGGGAGAGCAACGGCCAGCCCGAG |
| | | AACAACTACAAGACCACCCCCCCAGTGCTGGAC |
| | | AGCGACGGCAGCTTCTTCCTGTACAGCAGGCTG |
| | | ACCGTGGACAAGTCCAGATGGCAGGAGGGCAAC |
| | | GTCTTTAGCTGCTCCGTGATGCACGAGGCCCTG |
| | | CACAACCACTACACCCAGAAGAGCCTGAGCCTG |
| | | TCCCTGGGC |

BAP049-Clone-D LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 204 | VL | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 238 | DNA VL | GAGATCGTGCTGACCCAGTCCCCTGCCACCCTG |
| | | TCACTGTCTCCAGGCGAGAGAGCTACCCTGTCC |
| | | TGCAAGTCCTCCCAGTCCCTGCTGGACTCCGGC |
| | | AACCAGAAGAACTTCCTGACCTGGTATCAGCAG |
| | | AAGCCCGGCCAGGCCCCCAGACTGCTGATCTAC |
| | | TGGGCCTCCACCCGGGAATCTGGCGTGCCCTCT |
| | | AGATTCTCCGGCTCCGGCTCTGGCACCGACTTT |
| | | ACCTTCACCATCTCCAGCCTGGAAGCCGAGGAC |
| | | GCCGCCACCTACTACTGCCAGAACGACTACTCC |
| | | TACCCCTACACCTTCGGCCAGGGCACCAAGGTG |
| | | GAAATCAAG |
| SEQ ID NO: 206 | LC | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 239 | DNA LC | GAGATCGTGCTGACCCAGTCCCCTGCCACCCTG |
| | | TCACTGTCTCCAGGCGAGAGAGCTACCCTGTCC |
| | | TGCAAGTCCTCCCAGTCCCTGCTGGACTCCGGC |
| | | AACCAGAAGAACTTCCTGACCTGGTATCAGCAG |
| | | AAGCCCGGCCAGGCCCCCAGACTGCTGATCTAC |
| | | TGGGCCTCCACCCGGGAATCTGGCGTGCCCTCT |
| | | AGATTCTCCGGCTCCGGCTCTGGCACCGACTTT |
| | | ACCTTCACCATCTCCAGCCTGGAAGCCGAGGAC |
| | | GCCGCCACCTACTACTGCCAGAACGACTACTCC |
| | | TACCCCTACACCTTCGGCCAGGGCACCAAGGTG |
| | | GAAATCAAGCGTACGGTGGCCGCTCCCAGCGTG |
| | | TTCATCTTCCCCCCAAGCGACGAGCAGCTGAAG |
| | | AGCGGCACCGCCAGCGTGGTGTGTCTGCTGAAC |
| | | AACTTCTACCCCAGGGAGGCCAAGGTGCAGTGG |
| | | AAGGTGGACAACGCCCTGCAGAGCGGCAACAGC |
| | | CAGGAGAGCGTCACCGAGCAGGACAGCAAGGAC |
| | | TCCACCTACAGCCTGAGCAGCACCCTGACCCTG |
| | | AGCAAGGCCGACTACGAGAAGCACAAGGTGTAC |
| | | GCCTGTGAGGTGACCCACCAGGGCCTGTCCAGC |
| | | CCCGTGACCAAGAGCTTCAACAGGGGCGAGTGC |

BAP049-Clone-E HC

| | | |
|---|---|---|
| SEQ ID NO: 137 (Kabat) | HCDR1 | TYWMH |
| SEQ ID NO: 138 (Kabat) | HCDR2 | NIYPGTGGSNFDEKFKN |
| SEQ ID NO: 139 (Kabat) | HCDR3 | WTTGTGAY |
| SEQ ID NO: 140 (Chothia) | HCDR1 | GYTFTTY |
| SEQ ID NO: 141 (Chothia) | HCDR2 | YPGTGG |
| SEQ ID NO: 139 (Chothia) | HCDR3 | WTTGTGAY |
| SEQ ID NO: 172 | VH | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSS |
| SEQ ID NO: 229 | DNA VH | GAGGTGCAGCTGGTGCAGTCAGGCGCCGAAGTG |
| | | AAGAAGCCCGGCGAGTCACTGAGAATTAGCTGT |
| | | AAAGGTTCAGGCTACACCTTCACTACCTACTGG |
| | | ATGCACTGGGTCCGCCAGGCTACCGGTCAAGGC |
| | | CTCGAGTGGATGGGTAATATCTACCCCGGCACC |
| | | GGCGGCTCTAACTTCGACGAGAAGTTTAAGAAT |
| | | AGAGTGACTATCACCGCCGATAAGTCTACTAGC |
| | | ACCGCCTATATGGAACTGTCTAGCCTGAGATCA |
| | | GAGGACACCGCCGTCTACTACTGCACTAGGTGG |
| | | ACTACCGGCACAGGCGCCTACTGGGGTCAAGGC |
| | | ACTACCGTGACCGTGTCTAGC |
| SEQ ID NO: 225 | HC | EVQLVQSGAEVKKPGESLRISCKGSGYTFTTYW |
| | | MHWVRQATGQGLEWMGNIYPGTGGSNFDEKFKN |
| | | RVTITADKSTSTAYMELSSLRSEDTAVYYCTRW |
| | | TTGTGAYWGQGTTVTVSSASTKGPSVFPLAPCS |
| | | RSTSESTAALGCLVKDYFPEPVTVSWNSGALTS |
| | | GVHTFPAVLQSSGLYSLSSVVTVPSSSLGTKTY |
| | | TCNVDHKPSNTKVDKRVESKYGPPCPPCPAPEF |
| | | LGGPSVFLFPPKPKDTLMISRTPEVTCVVVDVS |
| | | QEDPEVQFNWYVDGVEVHNAKTKPREEQFNSTY |
| | | RVVSVLTVLHQDWLNGKEYKCKVSNKGLPSSIE |
| | | KTISKAKGQPREPQVYTLPPSQEEMTKNQVSLT |
| | | CLVKGFYPSDIAVEWESNGQPENNYKTTPPVLD |
| | | SDGSFFLYSRLTVDKSRWQEGNVFSCSVMHEAL |
| | | HNHYTQKSLSLSLG |
| SEQ ID NO: 230 | DNA HC | GAGGTGCAGCTGGTGCAGTCAGGCGCCGAAGTG |
| | | AAGAAGCCCGGCGAGTCACTGAGAATTAGCTGT |
| | | AAAGGTTCAGGCTACACCTTCACTACCTACTGG |
| | | ATGCACTGGGTCCGCCAGGCTACCGGTCAAGGC |
| | | CTCGAGTGGATGGGTAATATCTACCCCGGCACC |
| | | GGCGGCTCTAACTTCGACGAGAAGTTTAAGAAT |
| | | AGAGTGACTATCACCGCCGATAAGTCTACTAGC |
| | | ACCGCCTATATGGAACTGTCTAGCCTGAGATCA |
| | | GAGGACACCGCCGTCTACTACTGCACTAGGTGG |
| | | ACTACCGGCACAGGCGCCTACTGGGGTCAAGGC |
| | | ACTACCGTGACCGTGTCTAGCGCTAGCACTAAG |
| | | GGCCCGTCCGTGTTCCCCCTGGCACCTTGTAGC |
| | | CGGAGCACTAGCGAATCCACCGCTGCCCTCGGC |
| | | TGCCTGGTCAAGGATTACTTCCCGGAGCCCGTG |
| | | ACCGTGTCCTGGAACAGCGGAGCCCTGACCTCC |
| | | GGAGTGCACACCTTCCCCGCTGTGCTGCAGAGC |
| | | TCCGGGCTGTACTCGCTGTCGTCGGTGGTCACG |
| | | GTGCCTTCATCTAGCCTGGGTACCAAGACCTAC |
| | | ACTTGCAACGTGGACCACAAGCCTTCCAACACT |
| | | AAGGTGGACAAGCGCGTCGAATCGAAGTACGGC |
| | | CCACCGTGCCCGCCTTGTCCCGCGCCGGAGTTC |
| | | CTCGGCGGTCCCTCGGTCTTTCTGTTCCCACCG |
| | | AAGCCCAAGGACACTTTGATGATTTCCCGCACC |
| | | CCTGAAGTGACATGCGTGGTCGTGGACGTGTCA |
| | | CAGGAAGATCCGGAGGTGCAGTTCAATTGGTAC |
| | | GTGGATGGCGTCGAGGTGCACAACGCCAAAACC |
| | | AAGCCGAGGGAGGAGCAGTTCAACTCCACTTAC |
| | | CGCGTCGTGTCCGTGCTGACGGTGCTGCATCAG |
| | | GACTGGCTGAACGGGAAGGAGTACAAGTGCAAA |
| | | GTGTCCAACAAGGGACTTCCTAGCTCAATCGAA |
| | | AAGACCATCTCGAAAGCCAAGGGACAGCCCCGG |
| | | GAACCCCAAGTGTATACCCTGCCACCGAGCCAG |
| | | GAAGAAATGACTAAGAACCAAGTCTCATTGACT |
| | | TGCCTTGTGAAGGGCTTCTACCCATCGGATATC |
| | | GCCGTGGAATGGGAGTCCAACGGCCAGCCGGAA |
| | | AACAACTACAAGACCACCCCTCCGGTGCTGGAC |
| | | TCAGACGGATCCTTCTTCCTCTACTCGCGGCTG |
| | | ACCGTGGATAAGAGCAGATGGCAGGAGGGAAAT |
| | | GTGTTCAGCTGTTCTGTGATGCATGAAGCCCTG |
| | | CACAACCACTACACTCAGAAGTCCCTGTCCCTC |
| | | TCCCTGGGA |

BAP049-Clone-E LC

| | | |
|---|---|---|
| SEQ ID NO: 146 (Kabat) | LCDR1 | KSSQSLLDSGNQKNFLT |
| SEQ ID NO: 147 (Kabat) | LCDR2 | WASTRES |
| SEQ ID NO: 166 (Kabat) | LCDR3 | QNDYSYPYT |
| SEQ ID NO: 149 (Chothia) | LCDR1 | SQSLLDSGNQKNF |
| SEQ ID NO: 150 (Chothia) | LCDR2 | WAS |
| SEQ ID NO: 167 (Chothia) | LCDR3 | DYSYPY |
| SEQ ID NO: 204 | VL | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIK |
| SEQ ID NO: 240 | DNA VL | GAGATCGTCCTGACTCAGTCACCCGCTACCCTG |
| | | AGCCTGAGCCCTGGCGAGCGGGCTACACTGAGC |
| | | TGTAAATCTAGTCAGTCACTGCTGGATAGCGGT |
| | | AATCAGAAGAACTTCCTGACCTGGTATCAGCAG |
| | | AAGCCCGGTCAAGCCCCTAGACTGCTGATCTAC |
| | | TGGGCCTCTACTAGAGAATCAGGCGTGCCCTCT |
| | | AGGTTTAGCGGTAGCGGTAGTGGCACCGACTTC |
| | | ACCTTCACTATCTCTAGCCTGGAAGCCGAGGAC |
| | | GCCGCTACCTACTACTGTCAGAACGACTATAGC |
| | | TACCCCTACACCTTCGGTCAAGGCACTAAGGTC |
| | | GAGATTAAG |
| SEQ ID NO: 206 | LC | EIVLTQSPATLSLSPGERATLSCKSSQSLLDSG |
| | | NQKNFLTWYQQKPGQAPRLLIYWASTRESGVPS |
| | | RFSGSGSGTDFTFTISSLEAEDAATYYCQNDYS |
| | | YPYTFGQGTKVEIKRTVAAPSVFIFPPSDEQLK |
| | | SGTASVVCLLNNFYPREAKVQWKVDNALQSGNS |
| | | QESVTEQDSKDSTYSLSSTLTLSKADYEKHKVY |
| | | ACEVTHQGLSSPVTKSFNRGEC |
| SEQ ID NO: 241 | DNA LC | GAGATCGTCCTGACTCAGTCACCCGCTACCCTG |
| | | AGCCTGAGCCCTGGCGAGCGGGCTACACTGAGC |
| | | TGTAAATCTAGTCAGTCACTGCTGGATAGCGGT |
| | | AATCAGAAGAACTTCCTGACCTGGTATCAGCAG |
| | | AAGCCCGGTCAAGCCCCTAGACTGCTGATCTAC |
| | | TGGGCCTCTACTAGAGAATCAGGCGTGCCCTCT |
| | | AGGTTTAGCGGTAGCGGTAGTGGCACCGACTTC |
| | | ACCTTCACTATCTCTAGCCTGGAAGCCGAGGAC |
| | | GCCGCTACCTACTACTGTCAGAACGACTATAGC |
| | | TACCCCTACACCTTCGGTCAAGGCACTAAGGTC |
| | | GAGATTAAGCGTACGGTGGCCGCTCCCAGCGTG |
| | | TTCATCTTCCCCCCCAGCGACGAGCAGCTGAAG |
| | | AGCGGCACCGCCAGCGTGGTGTGCCTGCTGAAC |
| | | AACTTCTACCCCCGGGAGGCCAAGGTGCAGTGG |
| | | AAGGTGGACAACGCCCTGCAGAGCGGCAACAGC |
| | | CAGGAGAGCGTCACCGAGCAGGACAGCAAGGAC |
| | | TCCACCTACAGCCTGAGCAGCACCCTGACCCTG |
| | | AGCAAGGCCGACTACGAGAAGCATAAGGTGTAC |
| | | GCCTGCGAGGTGACCCACCAGGGCCTGTCCAGC |
| | | CCCGTGACCAAGAGCTTCAACAGGGGCGAGTGC |

BAP049 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 249 (Kabat) | LCDR3 | CAGAATGATTATAGTTATCCGTGCACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 252 (Chothia) | LCDR3 | GATTATAGTTATCCGTGC |

BAP049-chi HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-chi LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 249 (Kabat) | LCDR3 | CAGAATGATTATAGTTATCCGTGCACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 252 (Chothia) | LCDR3 | GATTATAGTTATCCGTGC |

BAP049-chi Y HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-chi Y LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum01 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| SEQ ID NO: 244 (Kabat) | HCDR3 | GATGAGAAGTTCAAGAAC |
| | | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum01 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum02 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum02 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum03 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum03 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum04 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum04 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum05 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum05 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum06 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum06 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum07 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum07 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum08 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum08 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum09 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum09 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum10 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum10 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum11 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum11 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum12 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum12 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum13 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum13 LC

| | | |
|---|---|---|
| SEQ ID NO: 285 (Kabat) | LCDR1 | AAGTCCAGTCAGAGTCTGTTAGACAGTGGAAAT |
| | | CAAAAGAACTTCTTAACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum14 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 255 (Kabat) | HCDR3 | TGGACTACTGGGACGGGAGCTTAC |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 255 (Chothia) | HCDR3 | TGGACTACTGGGACGGGAGCTTAC |

BAP049-hum14 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum15 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 255 (Kabat) | HCDR3 | TGGACTACTGGGACGGGAGCTTAC |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 255 (Chothia) | HCDR3 | TGGACTACTGGGACGGGAGCTTAC |

BAP049-hum15 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-hum16 HC

| | | |
|---|---|---|
| SEQ ID NO: 242 (Kabat) | HCDR1 | ACTTACTGGATGCAC |
| SEQ ID NO: 243 (Kabat) | HCDR2 | AATATTTATCCTGGTACTGGTGGTTCTAACTTC |
| | | GATGAGAAGTTCAAGAAC |
| SEQ ID NO: 244 (Kabat) | HCDR3 | TGGACTACTGGGACGGGAGCTTAT |
| SEQ ID NO: 245 (Chothia) | HCDR1 | GGCTACACATTCACCACTTAC |
| SEQ ID NO: 246 (Chothia) | HCDR2 | TATCCTGGTACTGGTGGT |
| SEQ ID NO: 244 (Chothia) | HCDR3 | TGGACTACTGGGACGGGAGCTTAT |

BAP049-hum16 LC

| | | |
|---|---|---|
| SEQ ID NO: 247 (Kabat) | LCDR1 | AAGTCCAGTCAGAGTCTGTTAGACAGTGGAAAT |
| | | CAAAAGAACTTCTTGACC |
| SEQ ID NO: 248 (Kabat) | LCDR2 | TGGGCATCCACTAGGGAATCT |
| SEQ ID NO: 253 (Kabat) | LCDR3 | CAGAATGATTATAGTTATCCGTACACG |
| SEQ ID NO: 250 (Chothia) | LCDR1 | AGTCAGAGTCTGTTAGACAGTGGAAATCAAAAG |
| | | AACTTC |
| SEQ ID NO: 251 (Chothia) | LCDR2 | TGGGCATCC |
| SEQ ID NO: 254 (Chothia) | LCDR3 | GATTATAGTTATCCGTAC |

BAP049-Clone-A HC

| | | |
|---|---|---|
| SEQ ID NO: 256 (Kabat) | HCDR1 | ACCTACTGGATGCAC |
| SEQ ID NO: 257 (Kabat) | HCDR2 | AACATCTATCCTGGCACCGGCGGCTCCAACTTC |
| | | GACGAGAAGTTCAAGAAC |
| SEQ ID NO: 258 (Kabat) | HCDR3 | TGGACAACCGGCACAGGCGCTTAT |
| SEQ ID NO: 259 (Chothia) | HCDR1 | GGCTACACCTTCACCACCTAC |
| SEQ ID NO: 260 (Chothia) | HCDR2 | TATCCTGGCACCGGCGGC |
| SEQ ID NO: 258 (Chothia) | HCDR3 | TGGACAACCGGCACAGGCGCTTAT |

BAP049-Clone-A LC

| | | |
|---|---|---|
| SEQ ID NO: 261 (Kabat) | LCDR1 | AAGTCCTCCCAGTCCCTGCTGGACTCCGGCAAC |
| | | CAGAAGAACTTCCTGACC |
| SEQ ID NO: 262 (Kabat) | LCDR2 | TGGGCCTCCACCCGGGAATCT |
| SEQ ID NO: 263 (Kabat) | LCDR3 | CAGAACGACTACTCCTACCCCTACACC |
| SEQ ID NO: 264 (Chothia) | LCDR1 | TCCCAGTCCCTGCTGGACTCCGGCAACCAGAAG |
| | | AACTTC |
| SEQ ID NO: 265 (Chothia) | LCDR2 | TGGGCCTCC |
| SEQ ID NO: 266 (Chothia) | LCDR3 | GACTACTCCTACCCCTAC |

BAP049-Clone-B HC

| | | |
|---|---|---|
| SEQ ID NO: 267 (Kabat) | HCDR1 | ACCTACTGGATGCAC |
| SEQ ID NO: 268 (Kabat) | HCDR2 | AATATCTACCCCGGCACCGGCGGCTCTAACTTC |
| | | GACGAGAAGTTTAAGAAT |
| SEQ ID NO: 269 (Kabat) | HCDR3 | TGGACTACCGGCACAGGCGCCTAC |
| SEQ ID NO: 270 (Chothia) | HCDR1 | GGCTACACCTTCACTACCTAC |
| SEQ ID NO: 271 (Chothia) | HCDR2 | TACCCCGGCACCGGCGGC |
| SEQ ID NO: 269 (Chothia) | HCDR3 | TGGACTACCGGCACAGGCGCCTAC |

BAP049-Clone-B LC

| | | |
|---|---|---|
| SEQ ID NO: 272 (Kabat) | LCDR1 | AAATCTAGTCAGTCACTGCTGGATAGCGGTAAT |
| | | CAGAAGAACTTCCTGACC |
| SEQ ID NO: 273 (Kabat) | LCDR2 | TGGGCCTCTACTAGAGAATCA |
| SEQ ID NO: 274 (Kabat) | LCDR3 | CAGAACGACTATAGCTACCCCTACACC |
| SEQ ID NO: 275 (Chothia) | LCDR1 | AGTCAGTCACTGCTGGATAGCGGTAATCAGAAG |
| | | AACTTC |
| SEQ ID NO: 276 (Chothia) | LCDR2 | TGGGCCTCT |
| SEQ ID NO: 277 (Chothia) | LCDR3 | GACTATAGCTACCCCTAC |

BAP049-Clone-C HC

| | | |
|---|---|---|
| SEQ ID NO: 256 (Kabat) | HCDR1 | ACCTACTGGATGCAC |
| SEQ ID NO: 257 (Kabat) | HCDR2 | AACATCTATCCTGGCACCGGCGGCTCCAACTTC |
| | | GACGAGAAGTTCAAGAAC |
| SEQ ID NO: 258 (Kabat) | HCDR3 | TGGACAACCGGCACAGGCGCTTAT |
| SEQ ID NO: 259 (Chothia) | HCDR1 | GGCTACACCTTCACCACCTAC |
| SEQ ID NO: 260 (Chothia) | HCDR2 | TATCCTGGCACCGGCGGC |
| SEQ ID NO: 258 (Chothia) | HCDR3 | TGGACAACCGGCACAGGCGCTTAT |

BAP049-Clone-C LC

| | | |
|---|---|---|
| SEQ ID NO: 261 (Kabat) | LCDR1 | AAGTCCTCCCAGTCCCTGCTGGACTCCGGCAAC |
| | | CAGAAGAACTTCCTGACC |
| SEQ ID NO: 262 (Kabat) | LCDR2 | TGGGCCTCCACCCGGGAATCT |
| SEQ ID NO: 263 (Kabat) | LCDR3 | CAGAACGACTACTCCTACCCCTACACC |
| SEQ ID NO: 264 (Chothia) | LCDR1 | TCCCAGTCCCTGCTGGACTCCGGCAACCAGAAG |
| | | AACTTC |
| SEQ ID NO: 265 (Chothia) | LCDR2 | TGGGCCTCC |
| SEQ ID NO: 266 (Chothia) | LCDR3 | GACTACTCCTACCCCTAC |

BAP049-Clone-D HC

| | | |
|---|---|---|
| SEQ ID NO: 256 (Kabat) | HCDR1 | ACCTACTGGATGCAC |
| SEQ ID NO: 278 (Kabat) | HCDR2 | AACATCTACCCTGGCACCGGCGGCTCCAACTTC |
| | | GACGAGAAGTTCAAGAAC |
| SEQ ID NO: 279 (Kabat) | HCDR3 | TGGACCACCGGAACCGGCGCCTAT |
| SEQ ID NO: 259 (Chothia) | HCDR1 | GGCTACACCTTCACCACCTAC |
| SEQ ID NO: 280 (Chothia) | HCDR2 | TACCCTGGCACCGGCGGC |
| SEQ ID NO: 279 (Chothia) | HCDR3 | TGGACCACCGGAACCGGCGCCTAT |

BAP049-Clone-D LC

| | | |
|---|---|---|
| SEQ ID NO: 261 (Kabat) | LCDR1 | AAGTCCTCCCAGTCCCTGCTGGACTCCGGCAAC |
| | | CAGAAGAACTTCCTGACC |
| SEQ ID NO: 262 (Kabat) | LCDR2 | TGGGCCTCCACCCGGGAATCT |
| SEQ ID NO: 263 (Kabat) | LCDR3 | CAGAACGACTACTCCTACCCCTACACC |
| SEQ ID NO: 264 (Chothia) | LCDR1 | TCCCAGTCCCTGCTGGACTCCGGCAACCAGAAG |
| | | AACTTC |
| SEQ ID NO: 265 (Chothia) | LCDR2 | TGGGCCTCC |
| SEQ ID NO: 266 (Chothia) | LCDR3 | GACTACTCCTACCCCTAC |

BAP049-Clone-E HC

| | | |
|---|---|---|
| SEQ ID NO: 267 (Kabat) | HCDR1 | ACCTACTGGATGCAC |
| SEQ ID NO: 268 (Kabat) | HCDR2 | AATATCTACCCCGGCACCGGCGGCTCTAACTTC |
| | | GACGAGAAGTTTAAGAAT |
| SEQ ID NO: 269 (Kabat) | HCDR3 | TGGACTACCGGCACAGGCGCCTAC |
| SEQ ID NO: 270 (Chothia) | HCDR1 | GGCTACACCTTCACTACCTAC |
| SEQ ID NO: 271 (Chothia) | HCDR2 | TACCCCGGCACCGGCGGC |
| SEQ ID NO: 269 (Chothia) | HCDR3 | TGGACTACCGGCACAGGCGCCTAC |

BAP049-Clone-E LC

| | | |
|---|---|---|
| SEQ ID NO: 272 (Kabat) | LCDR1 | AAATCTAGTCAGTCACTGCTGGATAGCGGTAAT |
| | | CAGAAGAACTTCCTGACC |
| SEQ ID NO: 273 (Kabat) | LCDR2 | TGGGCCTCTACTAGAGAATCA |
| SEQ ID NO: 274 (Kabat) | LCDR3 | CAGAACGACTATAGCTACCCCTACACC |
| SEQ ID NO: 275 (Chothia) | LCDR1 | AGTCAGTCACTGCTGGATAGCGGTAATCAGAAG |
| | | AACTTC |
| SEQ ID NO: 276 (Chothia) | LCDR2 | TGGGCCTCT |
| SEQ ID NO: 277 (Chothia) | LCDR3 | GACTATAGCTACCCCTAC |

In embodiments, an inhibitor of PD-1 is a molecule other than an antibody or fragment thereof. In embodiments, an inhibitior of PD-1 comprises a RNA molecule, e.g., dsRNA molecule, e.g., a a dsRNA molecule (e.g., an RNAi agents such as a shRNA, siRNA, miRNA, clustered regularly interspaced short palindromic repeats (CRISPR), transcription-activator like effector nuclease (TALEN), or zinc finger endonuclease (ZFN)) that targets and modulates or regulates, e.g., inhibits, PD-1, as described, e.g., in paragraph [00489] and Tables 16 and 17 of International Publication WO2015/090230, filed December 19, 2014, which is incorporated by reference in its entirety.

Antibodies, antibody fragments, and other inhibitors of PD-1, PD-L1 and PD-L2 are available in the art and may be used combination with a CAR-expressing cell of the present disclosure described herein. In some embodiments, the PD-1 inhibitor is chosen from PDR001 (Novartis), Nivolumab (Bristol-Myers Squibb), Pembrolizumab (Merck & Co), Pidilizumab (CureTech), MEDI0680 (Medimmune), REGN2810 (Regeneron), TSR-042 (Tesaro), PF-06801591 (Pfizer), BGB-A317 (Beigene), BGB-108 (Beigene), INCSHR1210 (Incyte), or AMP-224 (Amplimmune).

Nivolumab (also referred to as BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO2006/121168.

In some embodiments, the anti-PD-1 antibody is Nivolumab. Alternative names for Nivolumab include MDX-1106, MDX-1106-04, ONO-4538, OPDIVO® or BMS-936558. In some embodiments, the anti-PD-1 antibody is Nivolumab (CAS Registry Number: 946414-94-4). Nivolumab is a fully human IgG4 monoclonal antibody which specifically blocks PD1. Nivolumab (clone 5C4) and other human monoclonal antibodies that specifically bind to PD1 are disclosed in U.S. Pat. No. 8,008,449 and WO2006/121168. In one embodiment, the inhibitor of PD-1 is Nivolumab, and having a sequence disclosed herein (or a sequence substantially identical or similar thereto, e.g., a sequence at least 85%, 90%, 95% identical or higher to the sequence specified). In one embodiment, the anti-PD-1 antibody molecule comprises one or more of the CDR sequences (or collectively all of the CDR sequences), the heavy chain or light chain variable region sequence, or the heavy chain or light chain sequence of Nivolumab.

The heavy and light chain amino acid sequences of Nivolumab are as follows:

Heavy chain
(SEQ ID NO: 281)
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK
Light chain
(SEQ ID NO: 282)
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Pembrolizumab (formerly known as lambrolizumab, and also referred to as MK03475; Merck) is a humanized IgG4 monoclonal antibody that binds to PD-1. Pembrolizumab and other humanized anti-PD-1 antibodies are disclosed in U.S. Pat. No. 8,354,509 and WO2009/114335. AMP-224 (B7-DCIg; Amplimmune; e.g., disclosed in WO2010/027827 and WO2011/066342), is a PD-L2 Fc fusion soluble receptor that blocks the interaction between PD-1 and B7-H1. Other anti-PD-1 antibodies include AMP 514 (Amplimmune), among others, e.g., anti-PD-1 antibodies disclosed in U.S. Pat. No. 8,609,089, US 2010028330, and/or US 20120114649.

In some embodiments, the anti-PD-1 antibody is Pembrolizumab. Pembrolizumab (also referred to as Lambrolizumab, MK-3475, MK03475, SCH-900475 or KEYTRUDA®; Merck) is a humanized IgG4 monoclonal antibody that binds to PD-1. Pembrolizumab and other humanized anti-PD-1 antibodies are disclosed in Hamid, O. et al. (2013) New England Journal of Medicine 369 (2): 134-44, U.S. Pat. No. 8,354,509 and WO2009/114335.

### Pembrolizumab

In one embodiment, the inhibitor of PD-1 is Pembrolizumab disclosed in, e.g., U.S. Pat. No. 8,354,509 and WO 2009/114335, and having a sequence disclosed herein (or a sequence substantially identical or similar thereto, e.g., a sequence at least 85%, 90%, 95% identical or higher to the sequence specified). In one embodiment, the anti-PD-1 antibody molecule comprises one or more of the CDR sequences (or collectively all of the CDR sequences), the heavy chain or light chain variable region sequence, or the heavy chain or light chain sequence of Pembrolizumab.

In some embodiments, the anti-PD1 antibody molecule comprises:
(i) a heavy chain variable (VH) region comprising a VHCDR1 amino acid sequence of SEQ ID NO: 530; a VHCDR2 amino acid sequence of SEQ ID NO: 531; and a VHCDR3 amino acid sequence of SEQ ID NO: 532; and
(ii) a light chain variable (VL) region comprising a VLCDR1 amino acid sequence of SEQ ID NO: 527; a VLCDR2 amino acid sequence of SEQ ID NO: 528; and a VLCDR3 amino acid sequence of SEQ ID NO: 529,
   or a sequence similar thereto, e.g., a sequence at least 85%, 90%, 95% identical or higher.

In other embodiments, the anti-PD1 antibody molecule comprises a heavy chain comprising the amino acid of SEQ ID NO: 283, and a light chain comprising the amino acid of SEQ ID NO: 284, or a sequence identical or similar thereto, e.g., a sequence at least 85%, 90%, 95% identical or higher.

Amino acid sequences of the heavy chain, light chain, heavy chain CDRs, and light chain CDRs of Pembrolizumab are as disclosed below:

Heavy chain
(SEQ ID NO: 283)
QVQLVQSGVE VKKPGASVKV SCKASGYTFT NYYMYWVRQA PGQGLEWMGG 50
INPSNGGTNF NEKFKNRVTL TTDSSTTTAY MELKSLQFDD TAVYYCARRD 100
YRFDMGFDYW GQGTTVTVSS ASTKGPSVFP LAPCSRSTSE STAALGCLVK 150
DYFPEPVTVS WNSGALTSGV HTFPAVLQSS GLYSLSSVVT VPSSSLGTKT 200
YTCNVDHKPS NTKVDKRVES KYGPPCPPCP APEFLGGPSV FLFPPKPKDT 250
LMISRTPEVT CVVVDVSQED PEVQFNWYVD GVEVHNAKTK PREEQFNSTY 300
RVVSVLTVLH QDWLNGKEYK CKVSNKGLPS SIEKTISKAK GQPREPQVYT 350
LPPSQEEMTK NQVSLTCLVK GFYPSDIAVE WESNGQPENN YKTTPPVLDS 400
DGSFFLYSRL TVDKSRWQEG NVFSCSVMHE ALHNHYTQKS LSLSLGK 447
Light chain
(SEQ ID NO: 284)
EIVLTQSPAT LSLSPGERAT LSCRASKGVS TSGYSYLHWY QQKPGQAPRL 50
LIYLASYLES GVPARFSGSG SGTDFTLTIS SLEPEDFAVY YCQHSRDLPL 100
TFGGGTKVEI KRTVAAPSVF IFPPSDEQLK SGTASVVCLL NNFYPREAKV 150
QWKVDNALQS GNSQESVTEQ DSKDSTYSLS STLTLSKADY EKHKVYACEV 200
THQGLSSPVT KSFNRGEC 218
Light chain CDR1:
(SEQ ID NO: 527)
RASKGVSTSGYSYLH
Light chain CDR2:
(SEQ ID NO: 528)
LASYLES
Light chain CDR3:
(SEQ ID NO: 529)
QHSRDLPLT
Heavy chain CDR1:
(SEQ ID NO: 530)
NYYMY
Heavy chain CDR2:
(SEQ ID NO: 531)
GINPSNGGTNFNEKFKN
Heavy chain CDR3:
(SEQ ID NO: 532)
RDYRFDMGFDY

In some embodiments, the anti-PD-1 antibody is Pidilizumab. Pidilizumab (CT-011; Cure Tech) is a humanized IgGlk monoclonal antibody that binds to PD1. Pidilizumab and other humanized anti-PD-1 monoclonal antibodies are disclosed in WO2009/101611, Rosenblatt, J. etal. (2011) J Immunotherapy 34(5): 409-18, U.S. Pat. No. 7,695,715, U.S. Pat. No. 7,332,582, and U.S. Pat. No. 8,686,119, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of Pidilizumab.

In one embodiment, the anti-PD-1 antibody molecule is MEDI0680 (Medimmune), also known as AMP-514. MEDI0680 and other anti-PD-1 antibodies are disclosed in U.S. Pat. No. 9,205,148 and WO 2012/145493, incorporated by reference in their entirety. In one embodiment, the anti-PD-1 antibody molecule comprises one or more of the CDR sequences (or collectively all of the CDR sequences), the heavy chain or light chain variable region sequence, or the heavy chain or light chain sequence of MEDI0680.

In one embodiment, the anti-PD-1 antibody molecule is REGN2810 (Regeneron). In one embodiment, the anti-PD-1 antibody molecule comprises one or more of the CDR sequences (or collectively all of the CDR sequences), the heavy chain or light chain variable region sequence, or the heavy chain or light chain sequence of REGN2810.

In one embodiment, the anti-PD-1 antibody molecule is PF-06801591 (Pfizer). In one embodiment, the anti-PD-1 antibody molecule comprises one or more of the CDR sequences (or collectively all of the CDR sequences), the heavy chain or light chain variable region sequence, or the heavy chain or light chain sequence of PF-06801591.

In one embodiment, the anti-PD-1 antibody molecule is BGB-A317 or BGB-108 (Beigene). In one embodiment, the anti-PD-1 antibody molecule comprises one or more of the CDR sequences (or collectively all of the CDR sequences), the heavy chain or light chain variable region sequence, or the heavy chain or light chain sequence of BGB-A317 or BGB-108.

In one embodiment, the anti-PD-1 antibody molecule is INCSHR1210 (Incyte), also known as INCSHR01210 or SHR-1210. In one embodiment, the anti-PD-1 antibody molecule comprises one or more of the CDR sequences (or collectively all of the CDR sequences), the heavy chain or light chain variable region sequence, or the heavy chain or light chain sequence of INCSHR1210.

In one embodiment, the anti-PD-1 antibody molecule is TSR-042 (Tesaro), also known as ANB011. In one embodiment, the anti-PD-1 antibody molecule comprises one or more of the CDR sequences (or collectively all of the CDR sequences), the heavy chain or light chain variable region sequence, or the heavy chain or light chain sequence of TSR-042.

Other anti-PD1 antibodies include AMP 514 (Amplimmune), among others, e.g., anti-PD1 antibodies disclosed in U.S. Pat. No. 8,609,089, US 2010028330, and/or US 20120114649. Further known anti-PD-1 antibodies include those described, e.g., in WO 2015/112800, WO 2016/092419, WO 2015/085847, WO 2014/179664, WO 2014/194302, WO 2014/209804, WO 2015/200119, U.S. Pat. No. 8,735,553, U.S. Pat. No. 7,488,802, U.S. Pat. No. 8,927,697, U.S. Pat. No. 8,993,731, and U.S. Pat. No. 9,102,727, incorporated by reference in their entirety.

In one embodiment, the anti-PD-1 antibody is an antibody that competes for binding with, and/or binds to the same epitope on PD-1 as, one of the anti-PD-1 antibodies described herein.

In one embodiment, the PD-1 inhibitor is a peptide that inhibits the PD-1 signaling pathway, e.g., as described in U.S. Pat. No. 8,907,053, incorporated by reference in its entirety. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 inhibitor is AMP-224 (B7-DCIg; Amplimmune; e.g., disclosed in WO2010/027827 and WO2011/066342), is a PD-L2 Fc fusion soluble receptor that blocks the interaction between PD-1 and B7-H1.

In one embodiment, the anti-PD-1 antibody or fragment thereof is an anti-PD-1 antibody molecule as described in US 2015/0210769, entitled “Antibody Molecules to PD-1 and Uses Thereof,” incorporated by reference in its entirety. In one embodiment, the anti-PD-1 antibody molecule includes at least one, two, three, four, five or six CDRs (or collectively all of the CDRs) from a heavy and light chain variable region from an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or encoded by the nucleotide sequence in Table 1, or a sequence substantially identical (e.g., at least 80%, 85%, 90%, 92%, 95%, 97%, 98%, 99% or higher identical) to any of the aforesaid sequences; or closely related CDRs, e.g., CDRs which are identical or which have at least one amino acid alteration, but not more than two, three or four alterations (e.g., substitutions, deletions, or insertions, e.g., conservative substitutions).

In yet another embodiment, the anti-PD-1 antibody molecule comprises at least one, two, three or four variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or encoded by the nucleotide sequence in Table 1; or a sequence substantially identical (e.g., at least 80%, 85%, 90%, 92%, 95%, 97%, 98%, 99% or higher identical) to any of the aforesaid sequences.

### Therapeutic Application for Diseases and Disorders

Antigen, e.g., CD19, Associated Diseases and/or Disorders

The present disclosure provides compositions and methods for treating diseases and disorders (e.g., cancers), e.g., associated with the expression of an antigen, e.g., CD19. In one aspect, the invention provides methods for treating a disease wherein part of the cancer is negative for the antigen, e.g., CD19, and part of the cancer is positive for the antigen, e.g., CD19.

For example, the methods and compositions of the invention are useful for treating subjects that have relapsed or have a refractory disease (e.g., cancer, e.g., CD19+ cancer).

In certain embodiments, the subject has previously been administered a chemotherapy, e.g., a chemotherapy described herein (e.g., lymphodepleting chemotherapy, carboplatin, and/or gemcitabine), prior to administration with a CAR-expressing cell and/or a PD-1 inhibitor described herein. In embodiments, the subject has previously been administered an immunotherapy, e.g., an allogeneic bone marrow transplant, prior to administration with a CAR-expressing cell and/or a PD-1 inhibitor described herein. In embodiments, the subject has previously undergone radiation therapy prior to administration with a CAR-expressing cell and/or a PD-1 inhibitor described herein.

Exemplary cancers that can be treated with the combination therapy described herein (e.g., CAR-expressing cell and a PD-1 inhibitor) include a hematological cancer. Exemplary hematological cancers are described in greater detail below.

The disclosure includes (among other things) a type of cellular therapy where T cells are genetically modified to express a chimeric antigen receptor (CAR) and the CAR T cell is infused to a recipient in need thereof. The infused cell is able to kill tumor cells in the recipient. Unlike antibody therapies, CAR-modified T cells are able to replicate in vivo resulting in long-term persistence that can lead to sustained tumor control. In various aspects, the T cells administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell to the patient.

The invention also includes a type of cellular therapy where immune effector cells, e.g., NK cells or T cells are modified, e.g., by in vitro transcribed RNA, to transiently express a chimeric antigen receptor (CAR) and the CAR-expressing (e.g., CART) cell is infused to a recipient in need thereof. The infused cell is able to kill cancer cells in the recipient. Thus, in various aspects, the CAR-expressing cells, e.g., T cells, administered to the patient, is present for less than one month, e.g., three weeks, two weeks, one week, after administration of the CAR-expressing cell, e.g., T cell, to the patient.

Without wishing to be bound by any particular theory, the anti-cancer immunity response elicited by the CAR-modified T cells may be an active or a passive immune response, or alternatively may be due to a direct vs indirect immune response. In one aspect, the CAR (e.g., CD19-CAR) transduced T cells exhibit specific proinflammatory cytokine secretion and potent cytolytic activity in response to human cancer cells expressing the target antigen (e.g., CD19), resist soluble target antigen inhibition, mediate bystander killing and mediate regression of an established human cancer. For example, antigen-less cancer cells within a heterogeneous field of target antigen-expressing cancer may be susceptible to indirect destruction by target antigen-redirected T cells that has previously reacted against adjacent antigen-positive cancer cells.

In one aspect, the disclosure features a method of treating cancer in a subject. The method comprises administering to the subject a combination therapy that includes administering a CAR-expressing cell (e.g., CD19 CAR-expressing cell) and a PD-1 inhibitor such that the cancer is treated in the subject. An example of a cancer that is treatable by the combination therapy described herein is a cancer associated with expression of an antigen, e.g., CD19. In one aspect, the cancer associated with expression of an antigen, e.g., CD19, is selected from any of the hematological cancers described herein, e.g., a lymphoma, e.g., a follicular lymphoma or DLBCL.

In one embodiment, the combination therapy of a CAR-expressing cell (e.g., CD19 CAR-expressing cell) and a PD-1 inhibitor described herein results in one or more of: improved or increased anti-tumor activity of the CAR-expressing cell (e.g., CD19 CAR-expressing cell); increased proliferation or persistence of the CAR-expressing cell; improved or increased infiltration of the CAR-expressing cell; improved inhibition of tumor progression; delay of tumor progression; inhibition or reduction in cancer cell proliferation; and/or reduction in tumor burden, e.g., tumor volume, or size, e.g., as compared to a monotherapy of CAR-expressing cell or PD-1 inhibitor alone. In one embodiment, the combination therapy results in increased persistence of the CAR-expressing cell and a prolonged B cell recovery, e.g., manifested as a B cell aplasia. In one embodiment, the combination therapy results in increased persistence of the CAR-expressing cell and a lower, e.g., reduced, risk of relapse.

The present invention provides methods for inhibiting the proliferation of or reducing an antigen-expressing (e.g., CD19-expressing) cell population. In one embodiment, the methods comprise administering a combination therapy, e.g., a combination comprising a CAR-expressing cell (e.g., CD19 CAR-expressing cell), or a population of CAR expressing cells, and a PD-1 inhibitor. In certain embodiments, the combination therapy described herein reduces the quantity, number, amount or percentage of cells and/or cancer cells by at least at least 5% , 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% in a subject with or animal model of an antigen (e.g., CD19) or another cancer associated with antigen-expressing (e.g., CD19-expressing) cells relative to the quantity, number, amount, or percentage of cells and/or cancer cells in a subject treated with a CAR-expressing (e.g., CD19 CAR-expressing) cell or a PD-1 inhibitor alone. In one embodiment, the subject is a human. In an embodiment, the subject is a monkey, e.g., cynomolgus monkey.

The invention also provides methods for preventing, treating and/or managing a disorder, e.g., a disorder associated with antigen-expressing cells (e.g., CD19-expressing cells) (e.g., a cancer described herein), the methods comprising administering to a subject in need a CAR-expressing cell (e.g., CD19 CAR-expressing cell), or a population of CAR-expressing cells, and a PD-1 inhibitor. In one aspect, the subject is a human.

In one aspect, the invention pertains to a method of inhibiting growth of a cancer cell, (e.g., an antigen-expressing, e.g., CD19-expressing, cancer cell), comprising contacting the cancer cell with a CAR-expressing (e.g., CD19 CAR expressing) cell, e.g., a CD19 CART cell, described herein, and one or more other CAR expressing cells, e.g., as described herein, such that the CART is activated in response to the antigen and targets the cancer cell, wherein the growth of the cancer is inhibited. The CAR-expressing cell, e.g., T cell, is administered in combination with a PD-1, e.g., a PD-1 described herein.

The present disclosure also provides methods for preventing, treating and/or managing a disease, e.g., a disease associated with antigen-expressing (e.g., CD19-expressing) cells (e.g., a hematologic cancer or atypical cancer expressing the antigen, e.g., CD19), the methods comprising administering to a subject in need an CAR-expressing (e.g., CD19 CAR-expressing) cell that binds to the antigen-expressing cell and administering a PD-1 inhibitor described herein. In one aspect, the subject is a human. Non-limiting examples of disorders associated with antigen (e.g., CD19)-expressing cells include autoimmune disorders (such as lupus), inflammatory disorders (such as allergies and asthma) and cancers (such as hematological cancers or atypical cancers expressing the antigen, e.g., CD19).

The present disclosure also provides methods for preventing, treating and/or managing a disease associated with antigen-expressing (e.g.,CD19-expressing) cells, the methods comprising administering to a subject in need a CART cell (e.g., an anti-CD19 CART cell) of the invention that binds to the antigen-expressing (e.g., CD19-expressing) cell. In one aspect, the subject is a human.

The present disclosure also provides methods for preventing relapse of cancer, e.g., associated with antigen-expressing (e.g., CD19-expressing) cells, the methods comprising administering to a subject in need thereof a CART cell (e.g., an anti-CD19 CART cell) of the invention that binds to the antigen-expressing (e.g., CD19-expressing) cell. In one aspect, the methods comprise administering to the subject in need thereof an effective amount of a CART cell (e.g., an anti-CD19 CART cell) described herein that binds to the antigen-expressing (e.g., CD19-expressing) cell in combination with an effective amount of another therapy, e.g., PD-1 inhibitor.

Non-cancer related indications, e.g., associated with expression of an antigen, e.g., CD19, include, but are not limited to, e.g., autoimmune disease, (e.g., lupus), inflammatory disorders (allergy and asthma) and transplantation.

The CAR-expressing cells described herein may be administered either alone, or as a pharmaceutical composition in combination with diluents and/or with other components such as IL-2 or other cytokines or cell populations.

In some embodiments, a CAR-expressing cell (e.g., CD19 CAR-expressing cell) described herein is used to deplete a B cell (e.g., a population of B cells, e.g., regulatory B cells). Without wishing to be bound by theory, it is believed that depletion of B cells, e.g., regulatory B cells, can improve the tumor microenvironment such that combination therapies (e.g., combination therapies described herein) can be more effective (e.g., than without depletion of the B cells). Thus, provided herein is a method for reducing, e.g., depleting, regulatory cells (e.g., regulatory B cells). The method includes administering a CAR-expressing cell (e.g., CD19 CAR-expressing cell) described herein in an amount sufficient to reduce the regulatory cells. In some embodiments, the methods can be used to modulate a tumor microenvironment, e.g., to enhance the effectiveness of a therapy described herein.

### Hematologic Cancers

Hematological cancer conditions are the types of cancer such as leukemia, lymphoma and malignant lymphoproliferative conditions that affect blood, bone marrow and the lymphatic system.

In one embodiment, the hematologic cancer is leukemia. In one embodiment, the cancer is selected from the group consisting of one or more acute leukemias including but not limited to B-cell acute lymphoid leukemia (BALL), T-cell acute lymphoid leukemia (TALL), small lymphocytic leukemia (SLL), acute lymphoid leukemia (ALL); one or more chronic leukemias including but not limited to chronic myelogenous leukemia (CML), chronic lymphocytic leukemia (CLL); additional hematologic cancers or hematologic conditions including, but not limited to mantle cell lymphoma (MCL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells. Diseases associated with an antigen, e.g., CD19, expression include, but not limited to atypical and/or non-classical cancers, malignancies, precancerous conditions or proliferative diseases expressing the antigen, e.g., CD19; and any combination thereof.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

Lymphoma is a group of blood cell tumors that develop from lymphocytes. Exemplary lymphomas include non-Hodgkin lymphoma and Hodgkin lymphoma.

In an aspect, the invention pertains to a method of treating a mammal having Hodgkin lymphoma, comprising administering to the mammal an effective amount of the cells expressing CAR molecule, e.g., a CD19 CAR molecule, e.g., a CD19 CAR molecule described herein and a B-cell inhibitor.

In one aspect, the compositions and CART cells or CAR expressing NK cells of the present invention are particularly useful for treating B cell malignancies, such as non-Hodgkin lymphomas, e.g., DLBCL, Follicular lymphoma, or CLL.

Non-Hodgkin lymphoma (NHL) is a group of cancers of lymphocytes, formed from either B or T cells. NHLs occur at any age and are often characterized by lymph nodes that are larger than normal, weight loss, and fever. Different types of NHLs are categorized as aggressive (fast-growing) and indolent (slow-growing) types. B-cell non-Hodgkin lymphomas include Burkitt lymphoma, chronic lymphocytic leukemia/small lymphocytic lymphoma (CLL/SLL), diffuse large B-cell lymphoma (DLBCL), follicular lymphoma, immunoblastic large cell lymphoma, precursor B-lymphoblastic lymphoma, and mantle cell lymphoma. Examples of T-cell non-Hodgkin lymphomas include mycosis fungoides, anaplastic large cell lymphoma, and precursor T-lymphoblastic lymphoma. Lymphomas that occur after bone marrow or stem cell transplantation are typically B-cell non-Hodgkin lymphomas. See, e.g., Maloney. NEJM. 366.21(2012):2008-16. In some embodiments, non-Hodgkin lymphomas, e.g., DLBCL, Follicular lymphoma, or CLL, can have high expression of PD-L1, which can be linked to poor clinical outcomes.

Diffuse large B-cell lymphoma (DLBCL) is a form of NHL that develops from B cells. DLBCL is an aggressive lymphoma that can arise in lymph nodes or outside of the lymphatic system, e.g., in the gastrointestinal tract, testes, thyroid, skin, breast, bone, or brain. Three variants of cellular morphology are commonly observed in DLBCL: centroblastic, immunoblastic, and anaplastic. Centroblastic morphology is most common and has the appearance of medium-to-large-sized lymphocytes with minimal cytoplasm. There are several subtypes of DLBCL. For example, primary central nervous system lymphoma is a type of DLBCL that only affects the brain is called and is treated differently than DLBCL that affects areas outside of the brain. Another type of DLBCL is primary mediastinal B-cell lymphoma, which often occurs in younger patients and grows rapidly in the chest. Symptoms of DLBCL include a painless rapid swelling in the neck, armpit, or groin, which is caused by enlarged lymph nodes. For some subjects, the swelling may be painful. Other symptoms of DLBCL include night sweats, unexplained fevers, and weight loss. Although most patients with DLBCL are adults, this disease sometimes occurs in children.

In some embodiments, subsets of DLBCL patients show PD-L1 and/or PD-L2 locus alterations. For example, alterations of PD-L1 and PD-L2 loci was observed in 19% patients, with 12% patients showing copy number gains, 3% presenting amplifications and 4% showing translocations. In some embodiments, PD-L1 expression can be detected by immunohistochemistry (IHC) in samples from patients, including those with translocationr or amplifications of the PD-L1 and PD-L2 loci.

Genetic alterations can also be present in the non-GCB (Germinal center B-cell) subtype of DLBCL. In some embodiments, PD-L1 expression can be seen in non-GCB DLBCL patients. In some embodiments, non-GCB DLBCL patients resemble classical Hodgkin's lymphoma (cHL) in terms of PD-L1/PD-L2 expression or genetic alterations.

Treatment for DLBCL includes chemotherapy (e.g., cyclophosphamide, doxorubicin, vincristine, prednisone, etoposide),anti-neoplastic drugs (e.g., Pixantrone), antibodies (e.g., Rituxan), anthracycline-containing regimens, radiation, or stem cell transplants, e.g., autologous stem cell transplant (ASCT) or allogeneic hematopoietic stem cell transplant (HSCT). In some embodiments, treatment for DLBCL can include combination therapies including but not limited to: R-CHOP (cyclophosphamide, doxorubicin, vincristine, prednisone/prednisolone, and rituximab); R-ICE (Rituximab, ifosfamide, carboplatin and etoposide); R-DHAP (Rituximab, dexamethasone, cytarabine and cisplatin); R-GDP (Rituximab, dexamethasone, gemcitabine and cisplatin); GemOX (gemcitabine and oxaliplatin); or HDCT (high dose chemotherapy) and ASCT.

These treatments, e.g., lines of therapy, for DLBCL can be administered as a first line therapy, a second line therapy, a third line therapy or a fourth line therapy. In some embodiments, treatment for DLBCL can include one or more lines of therapy, e.g., one, two, three, or four lines of therapy. In some embodiments, the treatments for DLBCL can inclue any one or more of the treatments disclosed herein, or combinations thereof.

In some embodiments, a first line therapy comprises R-CHOP, R-ICE, R-DHAP, R-GDP, GemOx, Rituximab, HDCT and ASCT, Pixantrone, allogeneic HSCT, CART therapy (e.g., CTL019, CTL119 or BCMA CAR) or an investigative agent. In some embodiments, the first line therapy is R-CHOP.

In some embodiments, a second line therapy comprises R-CHOP, R-ICE, R-DHAP, R-GDP, GemOx, Rituximab, HDCT and ASCT, Pixantrone, allogeneic HSCT, CART therapy (e.g., CTL019, CTL119 or BCMA CAR) or an investigative agent. In some embodiments, the second line therapy comprises R-ICE, R-DHAP, R-GDP, GemOx, Rituximab, HDCT and ASCT, or investigative agents. In some embodiments, the second line therapy is R-ICE, R-DHAP or R-GDP. In some embodiments, the second line theraoy is HDCT in combination with ASCT. In some embodiments, the second line therapy is Rituximab. In some embodiments, the second line therapy is GemOx. In some embodiments, the second line therapy is an investigative agent.

In some embodiments, a third line therapy comprises R-CHOP, R-ICE, R-DHAP, R-GDP, GemOx, Rituximab, HDCT and ASCT, Pixantrone, allogeneic HSCT, CART therapy (e.g., CTL019, CTL119 or BCMA CAR) or an investigative agent. In some embodiments, a third line therapy is Pixantrone. In some embodiments, a third line therapy is an investigative agent. In some embodiments, the third line therapy is a CART therapy (e.g., CTL019, CTL119 or BCMA CAR). In other embodiments, the third line therapy is allogeneic HSCT.

In some embodiments, a fourth line therapy comprises R-CHOP, R-ICE, R-DHAP, R-GDP, GemOx, Rituximab, HDCT and ASCT, Pixantrone, allogeneic HSCT, CART therapy (e.g., CTL019, CTL119 or BCMA CAR) or an investigative agent. In some embodiments, the fourth line therapy comprises an investigative agent.

About 60% of patients respond to a Rituximab containing first line of therapy. In some embodiments, patients who receive more than two lines of therapy, e.g., two, three, or four lines of therapy have a poor prognosis. Patients recvieing R-DHAP and O-DHAP as second line therapy have a median progression free survival (PFS) of 2.1 and 1.8 months respectively, and a median overall survival (OS) of 13.2 and 13.7 months, respectively. Patients failing salvage therapy or relapsing after autologous HSCT have a median OS of 4.4 months. The 1 year OS of these patients is 23% and the 2 year OS for these patients is 15.7%. Additionally, there is no standard of care for third line chemotherapy, or for patients who fail autologous transplant or are ineligible for it. Therefore, there is an unmet need in r/r DLBCL.

CART therapy can potentially be curative, but not for all r/r DLBCL patients. Although CART therapy offers improved outcomes over existing therapies, about two thirds of r/r DLBCL patients will not have a durable response to CART therapy. A combination of CART therapy and checkpoint inhibitors, e.g., anti-PD-1 antibody (e.g., Pembrolizumab), can improve the response in r/r DLBCL patients.

In some embodiments, a combination of a CART therapy (e.g., CTL019, CTL119 or BCMA CAR) with a checkpoint inhibitor, e.g., an anti-PD-1 antibody (e.g., Pembrolizumab), can be used as a third line therapy. In some embodiments, the combination therapy can result in durable response rates in, e.g., patients with r/r DLBCL. In some embodiments, the combination therapy can prolong the persistence of the CART therapy (e.g., CTL019, CTL119 or BCMA CAR) at the tumor site (e.g., in the blood, bone marrow, or spleen). In other embodiments, the combination therapy can be better than a CART monotherapy, e.g., a monotherapy of CTL019, CTL119 or BCMA CAR. In some embodiments, the combination therapy can enhance the duration of response upon recovery of normal T cell populations in the subject, e.g., following lymphodepletion. In other embodiments, the anti-PD-1 antibody (e.g., Pembrolizumab) can block PD-1 mediated inhibition of a spontaneous immune response. In some embodiments, the subject receiving the combination therapy has DLBCL, e.g., GCB or non-GCB DLBCL. In some embodiments, the subject with DLBCL, e.g., GCB or non-GCB DLBCL, can be selected for combination therapy based on PD-L1 expression or genetic alterations.

Follicular lymphoma a type of non-Hodgkin lymphoma and is a lymphoma of follicle center B-cells (centrocytes and centroblasts), which has at least a partially follicular pattern. Follicular lymphoma cells express the B-cell markers CD10, CD19, CD20, and CD22. Follicular lymphoma cells are commonly negative for CDS. Morphologically, a follicular lymphoma tumor is made up of follicles containing a mixture of centrocytes (also called cleaved follicle center cells or small cells) and centroblasts (also called large noncleaved follicle center cells or large cells). The follicles are surrounded by non-malignant cells, mostly T-cells. The follicles contain predominantly centrocytes with a minority of centroblasts.The World Health Organization (WHO) morphologically grades the disease as follows: grade 1 (<5 centroblasts per high-power field (hpf); grade 2 (6-15 centroblasts/hpf); grade 3 (>15 centroblasts/hpf). Grade 3 is further subdivided into the following grades: grade 3A (centrocytes still present); grade 3B (the follicles consist almost entirely of centroblasts).

Treatment of follicular lymphoma includes chemotherapy, e.g., alkyating agents, nucleoside analogs, anthracycline-containing regimens, e.g., a combination therapy called CHOP—cyclophosphamide, doxorubicin, vincristine, prednisone/prednisolone, antibodies (e.g., rituximab), radioimmunotherapy, and hematopoietic stem cell transplantation.

CLL is a B-cell malignancy characterized by neoplastic cell proliferation and accumulation in bone morrow, blood, lymph nodes, and the spleen. The median age at time of diagnosis of CLL is about 65 years. Current treatments include chemotherapy, radiation therapy, biological therapy, or bone marrow transplantation. Sometimes symptoms are treated surgically (e.g., splenectomy removal of enlarged spleen) or by radiation therapy (e.g., de-bulking swollen lymph nodes). Chemotherapeutic agents to treat CLL include, e.g., fludarabine, 2-chlorodeoxyadenosine (cladribine), chlorambucil, vincristine, pentostatin, cyclophosphamide, alemtuzumab (Campath-1H), doxorubicin, and prednisone. Biological therapy for CLL includes antibodies, e.g., alemtuzumab, rituximab, and ofatumumab; as well as tyrosine kinase inhibitor therapies. A number of criteria can be used to classify stage of CLL, e.g., the Rai or Binet system. The Rai system describes CLL has having five stages: stage 0 where only lymphocytosis is present; stage I where lymphadenopathy is present; stage II where splenomegaly, lymphadenopathy, or both are present; stage III where anemia, organomegaly, or both are present (progression is defined by weight loss, fatigue, fever, massive organomegaly, and a rapidly increasing lymphocyte count); and stage IV where anemia, thrombocytopenia, organomegaly, or a combination thereof are present. Under the Binet staging system, there are three categories: stage A where lymphocytosis is present and less than three lymph nodes are enlarged (this stage is inclusive of all Rai stage 0 patients, one-half of Rai stage I patients, and one-third of Rai stage II patients); stage B where three or more lymph nodes are involved; and stage C wherein anemia or thrombocytopenia, or both are present. These classification systems can be combined with measurements of mutation of the immunoglobulin genes to provide a more accurate characterization of the state of the disease. The presence of mutated immunoglobulin genes correlates to improved prognosis.

In another embodiment, the CAR expressing cells of the present invention are used to treat cancers or leukemias, e.g., with leukemia stem cells. For example, the leukemia stem cells are CD34⁺/CD38⁻ leukemia cells.

### Combination Therapies

Any of the methods described herein may be used in combination with other known agents and therapies.

The combination described herein, e.g., a CAR-expressing cell (e.g., CD19 CAR-expressing cell) and a PD-1 inhibitor, and the at least one additional therapeutic agent can be administered simultaneously, in the same or in separate compositions, or sequentially. For sequential administration, the CAR-expressing cell and/or the PD-1 inhibitor described herein can be administered after the additional therapeutic agent, or the order of administration can be reversed where the additional therapeutic agent can be administered after the CAR-expressing cell and/or the PD-1 inhibitor described herein. Alternatively, the additional therapeutic agent can be administered between administration of the CAR-expressing cell and the PD-1 inhibitor.

In further aspects, the combination described herein, e.g., a CAR-expressing cell (e.g., CD19 CAR-expressing cell) and a PD-1 inhibitor, may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation. peptide vaccine, such as that described in Izumoto et al. 2008 J Neurosurg 108:963-971.

In one embodiment, the combination described herein, e.g., a CAR-expressing cell (e.g., CD19 CAR-expressing cell) and a PD-1 inhibitor, can be used in combination with a chemotherapeutic agent. Exemplary chemotherapeutic agents include an anthracycline (e.g., doxorubicin (e.g., liposomal doxorubicin)). a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General Chemotherapeutic agents are disclosed on pages 268-269 of International Application WO 2016/164731, filed Apr. 8, 2016, which is incorporated by reference in its entirety.

Exemplary alkylating agents are disclosed on pages 270-271 of International Application WO 2016/164731, filed Apr. 8, 2016, which is incorporated by reference in its entirety.

Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.0^{4.9}] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SEQ ID NO: 526) (SF1126, CAS 936487-67-1), and XL765.

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

In embodiments, the combination described herein, e.g., a CAR-expressing cell (e.g., CD19 CAR-expressing cell) and a PD-1 inhibitor, is administered to a subject in combination with brentuximab. Brentuximab is an antibody-drug conjugate of anti-CD30 antibody and monomethyl auristatin E. In embodiments, the subject has Hodgkin's lymphoma (HL), e.g., relapsed or refractory HL. In embodiments, the subject comprises CD30+ HL. In embodiments, the subject has undergone an autologous stem cell transplant (ASCT). In embodiments, the subject has not undergone an ASCT. In embodiments, brentuximab is administered at a dosage of about 1-3 mg/kg (e.g., about 1-1.5, 1.5-2, 2-2.5, or 2.5-3 mg/kg), e.g., intravenously, e.g., every 3 weeks.

In embodiments, the combination described herein, e.g., a CAR-expressing cell (e.g., CD19 CAR-expressing cell) and a PD-1 inhibitor, is administered to a subject in combination with brentuximab and dacarbazine or in combination with brentuximab and bendamustine. Dacarbazine is an alkylating agent with a chemical name of 5-(3,3-Dimethyl-1-triazenyl)imidazole-4-carboxamide. Bendamustine is an alkylating agent with a chemical name of 4-[5-[Bis(2-chloroethyl)amino]-1-methylbenzimidazol-2-yl]butanoic acid. In embodiments, the subject has Hodgkin's lymphoma (HL). In embodiments, the subject has not previously been treated with a cancer therapy. In embodiments, the subject is at least 60 years of age, e.g., 60, 65, 70, 75, 80, 85, or older. In embodiments, dacarbazine is administered at a dosage of about 300-450 mg/m² (e.g., about 300-325, 325-350, 350-375, 375-400, 400-425, or 425-450 mg/m²), e.g., intravenously. In embodiments, bendamustine is administered at a dosage of about 75-125 mg/m2 (e.g., 75-100 or 100-125 mg/m², e.g., about 90 mg/m²), e.g., intravenously. In embodiments, brentuximab is administered at a dosage of about 1-3 mg/kg (e.g., about 1-1.5, 1.5-2, 2-2.5, or 2.5-3 mg/kg), e.g., intravenously, e.g., every 3 weeks.

In some embodiments, a CAR-expressing cell described herein is administered to a subject in combination with a CD20 inhibitor, e.g., an anti-CD20 antibody (e.g., an anti-CD20 mono- or bispecific antibody) or a fragment thereof. Exemplary anti-CD20 antibodies include but are not limited to rituximab, ofatumumab, ocrelizumab, veltuzumab, obinutuzumab, TRU-015 (Trubion Pharmaceuticals), ocaratuzumab, and Pro131921 (Genentech). See, e.g., Lim et al. Haematologica. 95.1(2010):135-43.

In some embodiments, the anti-CD20 antibody comprises rituximab. Rituximab is a chimeric mouse/human monoclonal antibody IgG1 kappa that binds to CD20 and causes cytolysis of a CD20 expressing cell, e.g., as described in www.accessdata.fda.gov/drugsatfda_docs/label/2010/103705s5311lbl.pdf. In embodiments, a CAR-expressing cell described herein is administered to a subject in combination with rituximab. In embodiments, the subject has CLL or SLL.

In some embodiments, rituximab is administered intravenously, e.g., as an intravenous infusion. For example, each infusion provides about 500-2000 mg (e.g., about 500-550, 550-600, 600-650, 650-700, 700-750, 750-800, 800-850, 850-900, 900-950, 950-1000, 1000-1100, 1100-1200, 1200-1300, 1300-1400, 1400-1500, 1500-1600, 1600-1700, 1700-1800, 1800-1900, or 1900-2000 mg) of rituximab. In some embodiments, rituximab is administered at a dose of 150 mg/m² to 750 mg/m², e.g., about 150-175 mg/m², 175-200 mg/m², 200-225 mg/m², 225-250 mg/m², 250-300 mg/m², 300-325 mg/m², 325-350 mg/m², 350-375 mg/m², 375-400 mg/m², 400-425 mg/m², 425-450 mg/m², 450-475 mg/m², 475-500 mg/m², 500-525 mg/m², 525-550 mg/m², 550-575 mg/m², 575-600 mg/m², 600-625 mg/m², 625-650 mg/m², 650-675 mg/m², or 675-700 mg/m², where m² indicates the body surface area of the subject. In some embodiments, rituximab is administered at a dosing interval of at least 4 days, e.g., 4, 7, 14, 21, 28, 35 days, or more. For example, rituximab is administered at a dosing interval of at least 0.5 weeks, e.g., 0.5, 1, 2, 3, 4, 5, 6, 7, 8 weeks, or more. In some embodiments, rituximab is administered at a dose and dosing interval described herein for a period of time, e.g., at least 2 weeks, e.g., at least 2, 3, 4, 5, 6, 7, 8, 9, 10, 11, 12, 13, 14, 15, 16, 17, 18, 19, 20 weeks, or greater. For example, rituximab is administered at a dose and dosing interval described herein for a total of at least 4 doses per treatment cycle (e.g., at least 4, 5, 6, 7, 8, 9, 10, 11, 12, 13, 14, 15, 16, or more doses per treatment cycle).

In some embodiments, the anti-CD20 antibody comprises ofatumumab. Ofatumumab is an anti-CD20 IgGiK human monoclonal antibody with a molecular weight of approximately 149 kDa. For example, ofatumumab is generated using transgenic mouse and hybridoma technology and is expressed and purified from a recombinant murine cell line (NSO). See, e.g., www.accessdata.fda.gov/drugsatfda_docs/label/2009/125326lbl.pdf; and Clinical Trial Identifier number NCT01363128, NCT01515176, NCT01626352, and NCT01397591. In embodiments, a CAR-expressing cell described herein is administered to a subject in combination with ofatumumab. In embodiments, the subject has CLL or SLL.

In some embodiments, ofatumumab is administered as an intravenous infusion. For example, each infusion provides about 150-3000 mg (e.g., about 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1200, 1200-1400, 1400-1600, 1600-1800, 1800-2000, 2000-2200, 2200-2400, 2400-2600, 2600-2800, or 2800-3000 mg) of ofatumumab. In embodiments, ofatumumab is administered at a starting dosage of about 300 mg, followed by 2000 mg, e.g., for about 11 doses, e.g., for 24 weeks. In some embodiments, ofatumumab is administered at a dosing interval of at least 4 days, e.g., 4, 7, 14, 21, 28, 35 days, or more. For example, ofatumumab is administered at a dosing interval of at least 1 week, e.g., 1, 2, 3, 4, 5, 6, 7, 8, 9, 10, 11, 12, 24, 26, 28, 20, 22, 24, 26, 28, 30 weeks, or more. In some embodiments, ofatumumab is administered at a dose and dosing interval described herein for a period of time, e.g., at least 1 week, e.g., 1, 2, 3, 4, 5, 6, 7, 8, 9, 10, 11, 12, 13, 14, 15, 16, 17, 18, 19, 20, 22, 24, 26, 28, 30, 40, 50, 60 weeks or greater, or 1, 2, 3, 4, 5, 6, 7, 8, 9, 10, 11, 12 months or greater, or 1, 2, 3, 4, 5 years or greater. For example, ofatumumab is administered at a dose and dosing interval described herein for a total of at least 2 doses per treatment cycle (e.g., at least 2, 3, 4, 5, 6, 7, 8, 9, 10, 11, 12, 13, 14, 15, 16, 18, 20, or more doses per treatment cycle).

In some cases, the anti-CD20 antibody comprises ocrelizumab. Ocrelizumab is a humanized anti-CD20 monoclonal antibody, e.g., as described in Clinical Trials Identifier Nos. NCT00077870, NCT01412333, NCT00779220, NCT00673920, NCT01194570, and Kappos et al. Lancet. 19.378(2011):1779-87.

In some cases, the anti-CD20 antibody comprises veltuzumab. Veltuzumab is a humanized monoclonal antibody against CD20. See, e.g., Clinical Trial Identifier No. NCT00547066, NCT00546793, NCT01101581, and Goldenberg et al. Leuk Lymphoma. 51(5)(2010):747-55.

In some cases, the anti-CD20 antibody comprises GA101. GA101 (also called obinutuzumab or RO5072759) is a humanized and glyco-engineered anti-CD20 monoclonal antibody. See, e.g., Robak. Curr. Opin. Investig. Drugs. 10.6(2009):588-96; Clinical Trial Identifier Numbers: NCT01995669, NCT01889797, NCT02229422, and NCT01414205; and www.accessdata.fda.gov/drugsatfda_docs/label/2013/125486s000lbl.pdf.

In some cases, the anti-CD20 antibody comprises AME-133v. AME-133v (also called LY2469298 or ocaratuzumab) is a humanized IgG1 monoclonal antibody against CD20 with increased affinity for the FcγRIIIa receptor and an enhanced antibody dependent cellular cytotoxicity (ADCC) activity compared with rituximab. See, e.g., Robak et al. BioDrugs 25.1(2011):13-25; and Forero-Torres et al. Clin Cancer Res. 18.5(2012):1395-403.

In some cases, the anti-CD20 antibody comprises PRO131921. PRO131921 is a humanized anti-CD20 monoclonal antibody engineered to have better binding to FcγRIIIa and enhanced ADCC compared with rituximab. See, e.g., Robak et al. BioDrugs 25.1(2011):13-25; and Casulo et al. Clin Immunol. 154.1(2014):37-46; and Clinical Trial Identifier No. NCT00452127.

In some cases, the anti-CD20 antibody comprises TRU-015. TRU-015 is an anti-CD20 fusion protein derived from domains of an antibody against CD20. TRU-015 is smaller than monoclonal antibodies, but retains Fc-mediated effector functions. See, e.g., Robak et al. BioDrugs 25.1(2011):13-25. TRU-015 contains an anti-CD20 single-chain variable fragment (scFv) linked to human IgG1 hinge, CH2, and CH3 domains but lacks CH1 and CL domains.

In some embodiments, an anti-CD20 antibody described herein is conjugated or otherwise bound to a therapeutic agent, e.g., a chemotherapeutic agent (e.g., cytoxan, fludarabine, histone deacetylase inhibitor, demethylating agent, peptide vaccine, anti-tumor antibiotic, tyrosine kinase inhibitor, alkylating agent, anti-microtubule or anti-mitotic agent), anti-allergic agent, anti-nausea agent (or anti-emetic), pain reliever, or cytoprotective agent described herein.

In embodiments, a CAR-expressing cell described herein is administered to a subject in combination with a B-cell lymphoma 2 (BCL-2) inhibitor (e.g., venetoclax, also called ABT-199 or GDC-0199; ) and/or rituximab. In embodiments, a CAR-expressing cell described herein is administered to a subject in combination with venetoclax and rituximab. Venetoclax is a small molecule that inhibits the anti-apoptotic protein, BCL-2. Venetoclax has the chemical name: (4-(4-{[2-(4-chlorophenyl)-4,4-dimethylcyclohex-1-en-1-yl]methyl }piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide).

In embodiments, the subject has CLL. In embodiments, the subject has relapsed CLL, e.g., the subject has previously been administered a cancer therapy. In embodiments, venetoclax is administered at a dosage of about 15-600 mg (e.g., 15-20, 20-50, 50-75, 75-100, 100-200, 200-300, 300-400, 400-500, or 500-600 mg), e.g., daily. In embodiments, rituximab is administered at a dosage of about 350-550 mg/m2 (e.g., 350-375, 375-400, 400-425, 425-450, 450-475, or 475-500 mg/m2), e.g., intravenously, e.g., monthly.

In some embodiments, the combination described herein, e.g., a CAR-expressing cell (e.g., CD19 CAR-expressing cell) and a PD-1 inhibitor, is administered in combination with an oncolytic virus. In embodiments, oncolytic viruses are capable of selectively replicating in and triggering the death of or slowing the growth of a cancer cell. In some cases, oncolytic viruses have no effect or a minimal effect on non-cancer cells. An oncolytic virus includes but is not limited to an oncolytic adenovirus, oncolytic Herpes Simplex Viruses, oncolytic retrovirus, oncolytic parvovirus, oncolytic vaccinia virus, oncolytic Sinbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

In some embodiments, the oncolytic virus is a virus, e.g., recombinant oncolytic virus, described in US2010/0178684 A1, which is incorporated herein by reference in its entirety. In some embodiments, a recombinant oncolytic virus comprises a nucleic acid sequence (e.g., heterologous nucleic acid sequence) encoding an inhibitor of an immune or inflammatory response, e.g., as described in US2010/0178684 A1, incorporated herein by reference in its entirety. In embodiments, the recombinant oncolytic virus, e.g., oncolytic NDV, comprises a pro-apoptotic protein (e.g., apoptin), a cytokine (e.g., GM-CSF, interferon-gamma, interleukin-2 (IL-2), tumor necrosis factor-alpha), an immunoglobulin (e.g., an antibody against ED-B firbonectin), tumor associated antigen, a bispecific adapter protein (e.g., bispecific antibody or antibody fragment directed against NDV HN protein and a T cell co-stimulatory receptor, such as CD3 or CD28; or fusion protein between human IL-2 and single chain antibody directed against NDV HN protein). See, e.g., Zamarin et al. Future Microbiol. 7.3(2012):347-67, incorporated herein by reference in its entirety. In some embodiments, the oncolytic virus is a chimeric oncolytic NDV described in U.S. Pat. No. 8,591,881 B2, US 2012/0122185 A1, or US 2014/0271677 A1, each of which is incorporated herein by reference in their entireties.

In some embodiments, the oncolytic virus comprises a conditionally replicative adenovirus (CRAd), which is designed to replicate exclusively in cancer cells. See, e.g., Alemany et al. Nature Biotechnol. 18(2000):723-27. In some embodiments, an oncolytic adenovirus comprises one described in Table 1 on page 725 of Alemany et al., incorporated herein by reference in its entirety.

Exemplary oncolytic viruses include but are not limited to the following: Group B Oncolytic Adenovirus (ColoAd1) (PsiOxus Therapeutics Ltd.) (see, e.g., Clinical Trial Identifier: NCT02053220); ONCOS-102 (previously called CGTG-102), which is an adenovirus comprising granulocyte-macrophage colony stimulating factor (GM-CSF) (Oncos Therapeutics) (see, e.g., Clinical Trial Identifier: NCT01598129); VCN-01, which is a genetically modified oncolytic human adenovirus encoding human PH20 hyaluronidase (VCN Biosciences, S.L.) (see, e.g., Clinical Trial Identifiers: NCT02045602 and NCT02045589); Conditionally Replicative Adenovirus ICOVIR-5, which is a virus derived from wild-type human adenovirus serotype 5 (Had5) that has been modified to selectively replicate in cancer cells with a deregulated retinoblastoma/E2F pathway (Institut Català d'Oncologia) (see, e.g., Clinical Trial Identifier: NCT01864759); Celyvir, which comprises bone marrow-derived autologous mesenchymal stem cells (MSCs) infected with ICOVIR5, an oncolytic adenovirus (Hospital Infantil Universitario Niño Jesús, Madrid, Spain/Ramon Alemany) (see, e.g., Clinical Trial Identifier: NCT01844661); CG0070, which is a conditionally replicating oncolytic serotype 5 adenovirus (Ad5) in which human E2F-1 promoter drives expression of the essential E1a viral genes, thereby restricting viral replication and cytotoxicity to Rb pathway-defective tumor cells (Cold Genesys, Inc.) (see, e.g., Clinical Trial Identifier: NCT02143804); orDNX-2401 (formerly named Delta-24-RGD), which is an adenovirus that has been engineered to replicate selectively in retinoblastoma (Rb)-pathway deficient cells and to infect cells that express certain RGD-binding integrins more efficiently (Clinica Universidad de Navarra, Universidad de Navarra/DNAtrix, Inc.) (see, e.g., Clinical Trial Identifier: NCT01956734).

In some embodiments, an oncolytic virus described herein is administering by injection, e.g., subcutaneous, intra-arterial, intravenous, intramuscular, intrathecal, or intraperitoneal injection. In embodiments, an oncolytic virus described herein is administered intratumorally, transdermally, transmucosally, orally, intranasally, or via pulmonary administration. In an embodiment, cells expressing a CAR described herein are administered to a subject in combination with a molecule that decreases the Treg cell population. Methods that decrease the number of (e.g., deplete) Treg cells are known in the art and include, e.g., CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (e.g., Tregs) in the tumor microenvironment and reduces the subject's risk of relapse.

In one embodiment, the combination described herein, e.g., a CAR-expressing cell (e.g., CD19 CAR-expressing cell) and a PD-1 inhibitor, is administered to a subject in combination with a molecule targeting GITR and/or modulating GITR functions, such as a GITR agonist and/or a GITR antibody that depletes regulatory T cells (Tregs). In one embodiment, the GITR binding molecules and/or molecules modulating GITR functions (e.g., GITR agonist and/or Treg depleting GITR antibodies) are administered prior to the CAR-expressing cell. For example, in one embodiment, the GITR agonist can be administered prior to apheresis of the cells. In one embodiment, the subject has CLL. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. No. 7,812,135, U.S. Pat. No. 8,388,967, U.S. Pat. No. 8,591,886, European Patent No.: EP 1866339, PCT Publication No.: WO 2011/028683, PCT Publication No.:WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Patent No.: 7,618,632, and PCT Publication No.: WO 2011/051726.

In one embodiment, the combination described herein, e.g., a CAR-expressing cell (e.g., CD19 CAR-expressing cell) and a PD-1 inhibitor, is administered to a subject in combination with an mTOR inhibitor, e.g., an mTOR inhibitor described herein, e.g., a rapalog such as everolimus. In one embodiment, the mTOR inhibitor is administered prior to the CAR-expressing cell. For example, in one embodiment, the mTOR inhibitor can be administered prior to apheresis of the cells. In one embodiment, the subject has CLL.

In one embodiment, the combination described herein, e.g., a CAR-expressing cell (e.g., CD19 CAR-expressing cell) and a PD-1 inhibitor, is administered to a subject in combination with a GITR agonist, e.g., a GITR agonist described herein. In one embodiment, the GITR agonist is administered prior to the CAR-expressing cell. For example, in one embodiment, the GITR agonist can be administered prior to apheresis of the cells. In one embodiment, the subject has CLL.

In one embodiment, the combination described herein, e.g., a CAR-expressing cell (e.g., CD19 CAR-expressing cell) and a PD-1 inhibitor, is administered to a subject in combination with a protein tyrosine phosphatase inhibitor, e.g., a protein tyrosine phosphatase inhibitor described herein. In one embodiment, the protein tyrosine phosphatase inhibitor is an SHP-1 inhibitor, e.g., an SHP-1 inhibitor described herein, such as, e.g., sodium stibogluconate. In one embodiment, the protein tyrosine phosphatase inhibitor is an SHP-2 inhibitor.

In one embodiment, a CAR-expressing cell described herein can be used in combination with a kinase inhibitor. In one embodiment, the kinase inhibitor is a CDK4 inhibitor, e.g., a CDK4 inhibitor described herein, e.g., a CDK4/6 inhibitor, such as, e.g., 6-Acetyl-8-cyclopentyl-5-methyl-2-(5-piperazin-1-yl-pyridin-2-ylamino)-8H-pyrido[2,3-d]pyrimidin-7-one, hydrochloride (also referred to as palbociclib or PD0332991). In one embodiment, the kinase inhibitor is a BTK inhibitor, e.g., a BTK inhibitor described herein, such as, e.g., ibrutinib. In one embodiment, the kinase inhibitor is an mTOR inhibitor, e.g., an mTOR inhibitor described herein, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor described herein. In one embodiment, the kinase inhibitor is a MNK inhibitor, e.g., a MNK inhibitor described herein, such as, e.g., 4-amino-5-(4-fluoroanilino)-pyrazolo [3,4-d] pyrimidine. The MNK inhibitor can be, e.g., a MNK1a, MNK1b, MNK2a and/or MNK2b inhibitor. In one embodiment, the kinase inhibitor is a dual PI3K/mTOR inhibitor described herein, such as, e.g., PF-04695102.

In one embodiment, the kinase inhibitor is a CDK4 inhibitor selected from aloisine A; flavopiridol or HMR-1275, 2-(2-chlorophenyl)-5,7-dihydroxy-8-[(3S,4R)-3-hydroxy-1-methyl-4-piperidinyl]-4-chromenone; crizotinib (PF-02341066; 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265); indisulam (E7070); roscovitine (CYC202); palbociclib (PD0332991); dinaciclib (SCH727965); N-[5-[[(5-tert-butyloxazol-2-yl)methyl]thio]thiazol-2-yl]piperidine-4-carboxamide (BMS 387032); 4-[[9-chloro-7-(2,6-difluorophenyl)-5H-pyrimido[5,4-d][2]benzazepin-2-yl]amino]-benzoic acid (MLN8054); 5-[3-(4,6-difluoro-1H-benzimidazol-2-yl)-1H-indazol-5-yl]-N-ethyl-4-methyl-3-pyridinemethanamine (AG-024322); 4-(2,6-dichlorobenzoylamino)-1H-pyrazole-3-carboxylic acid N-(piperidin-4-yl)amide (AT7519); 4-[2-methyl-1-(1-methylethyl)-1H-imidazol-5-yl]-N-[4-(methylsulfonyl)phenyl]-2-pyrimidinamine (AZD5438); and XL281 (BMS908662).

In one embodiment, the kinase inhibitor is a CDK4 inhibitor, e.g., palbociclib (PD0332991), and the palbociclib is administered at a dose of about 50 mg, 60 mg, 70 mg, 75 mg, 80 mg, 90 mg, 100 mg, 105 mg, 110 mg, 115 mg, 120 mg, 125 mg, 130 mg, 135 mg (e.g., 75 mg, 100 mg or 125 mg) daily for a period of time, e.g., daily for 14-21 days of a 28 day cycle, or daily for 7-12 days of a 21 day cycle. In one embodiment, 1, 2, 3, 4, 5, 6, 7, 8, 9, 10, 11, 12 or more cycles of palbociclib are administered.

In embodiments, the combination described herein, e.g., a CAR-expressing cell (e.g., CD19 CAR-expressing cell) and a PD-1 inhibitor, is administered to a subject in combination with a cyclin-dependent kinase (CDK) 4 or 6 inhibitor, e.g., a CDK4 inhibitor or a CDK6 inhibitor described herein. In embodiments, a CAR-expressing cell described herein is administered to a subject in combination with a CDK4/6 inhibitor (e.g., an inhibitor that targets both CDK4 and CDK6), e.g., a CDK4/6 inhibitor described herein. In an embodiment, the subject has MCL. MCL is an aggressive cancer that is poorly responsive to currently available therapies, i.e., essentially incurable. In many cases of MCL, cyclin D1 (a regulator of CDK4/6) is expressed (e.g., due to chromosomal translocation involving immunoglobulin and Cyclin D1 genes) in MCL cells. Thus, without being bound by theory, it is thought that MCL cells are highly sensitive to CDK4/6 inhibition with high specificity (i.e., minimal effect on normal immune cells). CDK4/6 inhibitors alone have had some efficacy in treating MCL, but have only achieved partial remission with a high relapse rate. An exemplary CDK4/6 inhibitor is LEE011 (also called ribociclib), the structure of which is shown below.

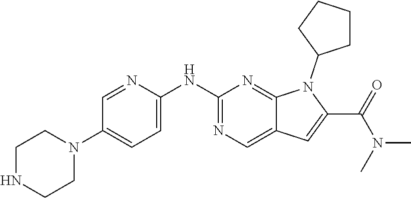 [SMILES: CN(C)C(=O)c1cc2cnc(Nc3ccc(N4CCNCC4)cn3)nc2n1C1CCCC1]

Without being bound by theory, it is believed that administration of a CAR-expressing cell described herein with a CDK4/6 inhibitor (e.g., LEE011 or other CDK4/6 inhibitor described herein) can achieve higher responsiveness, e.g., with higher remission rates and/or lower relapse rates, e.g., compared to a CDK4/6 inhibitor alone.

In one embodiment, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In a preferred embodiment, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In one embodiment, the kinase inhibitor is a BTK inhibitor, e.g., ibrutinib (PCI-32765). In embodiments, a CAR-expressing cell described herein is administered to a subject in combination with a BTK inhibitor (e.g., ibrutinib). In embodiments, a CAR-expressing cell described herein is administered to a subject in combination with ibrutinib (also called PCI-32765). Ibrutinib has the chemical name: (1-[(3R)-3-[4-Amino-3-(4-phenoxyphenyl)-1H-pyrazolo[3,4-d]pyrimidin-1-yl]piperidin-1-yl]prop-2-en-1-one).

In embodiments, the subject has CLL, mantle cell lymphoma (MCL), or small lymphocytic lymphoma (SLL). For example, the subject has a deletion in the short arm of chromosome 17 (del(17p), e.g., in a leukemic cell). In other examples, the subject does not have a del(17p). In embodiments, the subject has relapsed CLL or SLL, e.g., the subject has previously been administered a cancer therapy (e.g., previously been administered one, two, three, or four prior cancer therapies). In embodiments, the subject has refractory CLL or SLL. In other embodiments, the subject has follicular lymphoma, e.g., relapse or refractory follicular lymphoma. In some embodiments, ibrutinib is administered at a dosage of about 300-600 mg/day (e.g., about 300-350, 350-400, 400-450, 450-500, 500-550, or 550-600 mg/day, e.g., about 420 mg/day or about 560 mg/day), e.g., orally. In embodiments, the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle cycle, or daily for 28 day cycle. In one embodiment, 1, 2, 3, 4, 5, 6, 7, 8, 9, 10, 11, 12 or more cycles of ibrutinib are administered.

In some embodiments, ibrutinib is administered in combination with rituximab. See, e.g., Burger et al. (2013) Ibrutinib In Combination With Rituximab (iR) Is Well Tolerated and Induces a High Rate Of Durable Remissions In Patients With High-Risk Chronic Lymphocytic Leukemia (CLL): New, Updated Results Of a Phase II Trial In 40 Patients, Abstract 675 presented at 55^{th} ASH Annual Meeting and Exposition, New Orleans, La. 7-10 December. Without being bound by theory, it is thought that the addition of ibrutinib enhances the T cell proliferative response and may shift T cells from a T-helper-2 (Th2) to T-helper-1 (Th1) phenotype. Th1 and Th2 are phenotypes of helper T cells, with Th1 versus Th2 directing different immune response pathways. A Th1 phenotype is associated with proinflammatory responses, e.g., for killing cells, such as intracellular pathogens/viruses or cancerous cells, or perpetuating autoimmune responses. A Th2 phenotype is associated with eosinophil accumulation and anti-inflammatory responses.

In some embodiments of the methods, uses, and compositions herein, the BTK inhibitor is a BTK inhibitor described in International Application WO/2015/079417, which is herein incorporated by reference in its entirety. For instance, in some embodiments, the BTK inhibitor is a compound of formula (I) or a pharmaceutically acceptable salt thereof;

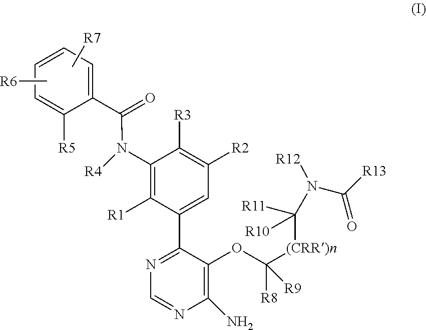

wherein,

R1 is hydrogen, C1-C6 alkyl optionally substituted by hydroxy;

R2 is hydrogen or halogen;

R3 is hydrogen or halogen;

R4 is hydrogen;

R5 is hydrogen or halogen;

or R4 and R5 are attached to each other and stand for a bond, —CH2-, —CH2-CH2-, —CH═CH—, —CH═CH—CH2-; —CH2-CH═CH—; or —CH2-CH2-CH2-;

R6 and R7 stand independently from each other for H, C1-C6 alkyl optionally substituted by hydroxyl, C3-C6 cycloalkyl optionally substituted by halogen or hydroxy, or halogen;

R8, R9, R, R′, R10 and R11 independently from each other stand for H, or C1-C6 alkyl optionally substituted by C1-C6 alkoxy; or any two of R8, R9, R, R′, R10 and R11 together with the carbon atom to which they are bound may form a 3-6 membered saturated carbocyclic ring;

R12 is hydrogen or C1-C6 alkyl optionally substituted by halogen or C1-C6 alkoxy;

or R12 and any one of R8, R9, R, R′, R10 or R11 together with the atoms to which they are bound may form a 4, 5, 6 or 7 membered azacyclic ring, which ring may optionally be substituted by halogen, cyano, hydroxyl, C1-C6 alkyl or C1-C6 alkoxy;

n is0 or 1; and

R13 is C2-C6 alkenyl optionally substituted by C1-C6 alkyl, C1-C6 alkoxy or N,N-di-C1-C6 alkyl amino; C2-C6 alkynyl optionally substituted by C1-C6 alkyl or C1-C6 alkoxy; or C2-C6 alkylenyl oxide optionally substituted by C1-C6 alkyl.

In some embodiments, the BTK inhibitor of Formula I is chosen from: N-(3-(5-((1-Acryloylazetidin-3-yl)oxy)-6-aminopyrimidin-4-yl)-5-fluoro-2-methylphenyl)-4-cyclopropyl-2-fluorobenzamide; (E)-N-(3-(6-Amino-5-((1-(but-2-enoyl)azetidin-3-yl)oxy)pyrimidin-4-yl)-5-fluoro-2-methylphenyl)-4-cyclopropyl-2-fluorobenzamide; N-(3-(6-Amino-5-((1-propioloylazetidin-3-yl)oxy)pyrimidin-4-yl)-5-fluoro-2-methylphenyl)-4-cyclopropyl-2-fluorobenzamide; N-(3-(6-Amino-5-((1-(but-2-ynoyl)azetidin-3-yl)oxy)pyrimidin-4-yl)-5-fluoro-2-methylphenyl)-4-cyclopropyl-2-fluorobenzamide; N-(3-(5-((1-Acryloylpiperidin-4-yl)oxy)-6-aminopyrimidin-4-yl)-5-fluoro-2-methylphenyl)-4-cyclopropyl-2-fluorobenzamide; N-(3-(6-Amino-5-(2-(N-methylacrylamido)ethoxy)pyrimidin-4-yl)-5-fluoro-2-methylphenyl)-4-cyclopropyl-2-fluorobenzamide; (E)-N-(3-(6-Amino-5-(2-(N-methylbut-2-enamido)ethoxy)pyrimidin-4-yl)-5-fluoro-2-methylphenyl)-4-cyclopropyl-2-fluorobenzamide; N-(3-(6-Amino-5-(2-(N-methylpropiolamido)ethoxy)pyrimidin-4-yl)-5-fluoro-2-methylphenyl)-4-cyclopropyl-2-fluorobenzamide; (E)-N-(3-(6-Amino-5-(2-(4-methoxy-N-methylbut-2-enamido)ethoxy)pyrimidin-4-yl)-5-fluoro-2-methylphenyl)-4-cyclopropyl-2-fluorobenzamide; N-(3-(6-Amino-5-(2-(N-methylbut-2-ynamido)ethoxy)pyrimidin-4-yl)-5-fluoro-2-methylphenyl)-4-cyclopropyl-2-fluorobenzamide; N-(2-((4-Amino-6-(3-(4-cyclopropyl-2-fluorobenzamido)-5-fluoro-2-methylphenyl)pyrimidin-5-yl)oxy)ethyl)-N-methyloxirane-2-carboxamide; N-(2-((4-Amino-6-(3-(6-cyclopropyl-8-fluoro-1-oxoisoquinolin-2(1H)-yl)phenyl)pyrimidin-5-yl)oxy)ethyl)-N-methylacrylamide; N-(3-(5-(2-Acrylamidoethoxy)-6-aminopyrimidin-4-yl)-5-fluoro-2-methylphenyl)-4-cyclopropyl-2-fluorobenzamide; N-(3-(6-Amino-5-(2-(N-ethylacrylamido)ethoxy)pyrimidin-4-yl)-5-fluoro-2-methylphenyl)-4-cyclopropyl-2-fluorobenzamide; N-(3-(6-Amino-5-(2-(N-(2-fluoroethyl)acrylamido)ethoxy)pyrimidin-4-yl)-5-fluoro-2-methylphenyl)-4-cyclopropyl-2-fluorobenzamide; N-(3-(5-((1-Acrylamidocyclopropyl)methoxy)-6-aminopyrimidin-4-yl)-5-fluoro-2-methylphenyl)-4-cyclopropyl-2-fluorobenzamide; (S)-N-(3-(5-(2-Acrylamidopropoxy)-6-aminopyrimidin-4-yl)-5-fluoro-2-methylphenyl)-4-cyclopropyl-2-fluorobenzamide; (S)-N-(3-(6-Amino-5-(2-(but-2-ynamido)propoxy)pyrimidin-4-yl)-5-fluoro-2-methylphenyl)-4-cyclopropyl-2-fluorobenzamide; (S)-N-(3-(6-Amino-5-(2-(N-methylacrylamido)propoxy)pyrimidin-4-yl)-5-fluoro-2-methylphenyl)-4-cyclopropyl-2-fluorobenzamide; (S)-N-(3-(6-Amino-5-(2-(N-methylbut-2-ynamido)propoxy)pyrimidin-4-yl)-5-fluoro-2-methylphenyl)-4-cyclopropyl-2-fluorobenzamide; N-(3-(6-Amino-5-(3-(N-methylacrylamido)propoxy)pyrimidin-4-yl)-5-fluoro-2-methylphenyl)-4-cyclopropyl-2-fluorobenzamide; (S)-N-(3-(5-((1-Acryloylpyrrolidin-2-yl)methoxy)-6-aminopyrimidin-4-yl)-5-fluoro-2-methylphenyl)-4-cyclopropyl-2-fluorobenzamide; (S)-N-(3-(6-Amino-5-((1-(but-2-ynoyl)pyrrolidin-2-yl)methoxy)pyrimidin-4-yl)-5-fluoro-2-methylphenyl)-4-cyclopropyl-2-fluorobenzamide; (S)-2-(3-(5-((1-Acryloylpyrrolidin-2-yl)methoxy)-6-aminopyrimidin-4-yl)-5-fluoro-2-(hydroxymethyl)phenyl)-6-cyclopropyl-3,4-dihydroisoquinolin-1(2H)-one; N-(2-((4-Amino-6-(3-(6-cyclopropyl-1-oxo-3,4-dihydroisoquinolin-2(1H)-yl)-5-fluoro-2-(hydroxymethyl)phenyl)pyrimidin-5-yl)oxy)ethyl)-N-methylacrylamide; N-(3-(5-(((2S,4R)-1-Acryloyl-4-methoxypyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4R)-1-(but-2-ynoyl)-4-methoxypyrrolidin-2-yl)methoxy)pyrimidin-4-yl)-5-fluoro-2-methylphenyl)-4-cyclopropyl-2-fluorobenzamide; 2-(3-(5-(((2S,4R)-1-Acryloyl-4-methoxypyrrolidin-2-yl)methoxy)-6-aminopyrimidin-4-yl)-5-fluoro-2-(hydroxymethyl)phenyl)-6-cyclopropyl-3,4-dihydroisoquinolin-1(2H)-one; N-(3-(5-(((2S,4S)-1-Acryloyl-4-methoxypyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4S)-1-(but-2-ynoyl)-4-methoxypyrrolidin-2-yl)methoxy)pyrimidin-4-yl)-5-fluoro-2-methylphenyl)-4-cyclopropyl-2-fluorobenzamide; N-(3-(5-(((2S,4R)-1-Acryloyl-4-fluoropyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4R)-1-(but-2-ynoyl)-4-fluoropyrrolidin-2-yl)methoxy)pyrimidin-4-yl)-5-fluoro-2-methylphenyl)-4-cyclopropyl-2-fluorobenzamide; (S)-N-(3-(5-((1-Acryloylazetidin-2-yl)methoxy)-6-aminopyrimidin-4-yl)-5-fluoro-2-methylphenyl)-4-cyclopropyl-2-fluorobenzamide; (S)-N-(3-(6-Amino-5-((1-propioloylazetidin-2-yl)methoxy)pyrimidin-4-yl)-5-fluoro-2-methylphenyl)-4-cyclopropyl-2-fluorobenzamide; (S)-2-(3-(5-((1-Acryloylazetidin-2-yl)methoxy)-6-aminopyrimidin-4-yl)-5-fluoro-2-(hydroxymethyl)phenyl)-6-cyclopropyl-3,4-dihydroisoquinolin-1(2H)-one; (R)-N-(3-(5-((1-Acryloylazetidin-2-yl)methoxy)-6-aminopyrimidin-4-yl)-5-fluoro-2-methylphenyl)-4-cyclopropyl-2-fluorobenzamide; (R)-N-(3-(5-((1-Acryloylpiperidin-3-yl)methoxy)-6-aminopyrimidin-4-yl)-5-fluoro-2-methylphenyl)-4-cyclopropyl-2-fluorobenzamide; N-(3-(5-(((2R,3S)-1-Acryloyl-3-methoxypyrrolidin-2-yl)methoxy)-6-aminopyrimidin-4-yl)-5-fluoro-2-methylphenyl)-4-cyclopropyl-2-fluorobenzamide; N-(3-(5-(((2S,4R)-1-Acryloyl-4-cyanopyrrolidin-2-yl)methoxy)-6-aminopyrimidin-4-yl)-5-fluoro-2-methylphenyl)-4-cyclopropyl-2-fluorobenzamide; or N-(3-(5-(((2S,4S)-1-Acryloyl-4-cyanopyrrolidin-2-yl)methoxy)-6-aminopyrimidin-4-yl)-5-fluoro-2-methylphenyl)-4-cyclopropyl-2-fluorobenzamide.

Unless otherwise provided, the chemical terms used above in describing the BTK inhibitor of Formula I are used according to their meanings as set out in International Application WO/2015/079417, which is herein incorporated by reference in its entirety.

In one embodiment, the kinase inhibitor is an mTOR inhibitor selected from temsirolimus; ridaforolimus (1R,2R,4S)-4-[(2R)-2[(1R,9S,12S,15R,16E,18R,19R,21R, 23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4.9}] hexatriaconta-16,24,26,28-tetraen-12-yl]propyl]-2-methoxycyclohexyl dimethylphosphinate, also known as AP23573 and MK8669; everolimus (RAD001); rapamycin (AY22989); simapimod; (5-{2,4-bis[(3S)-3-methylmorpholin-4-yl]pyrido[2,3-d]pyrimidin-7-yl}-2-methoxyphenyl)methanol (AZD8055); 2-amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SEQ ID NO: 526) (SF1126); and XL765.

In one embodiment, the kinase inhibitor is an mTOR inhibitor, e.g., rapamycin, and the rapamycin is administered at a dose of about 3 mg, 4 mg, 5 mg, 6 mg, 7 mg, 8 mg, 9 mg, 10 mg (e.g., 6 mg) daily for a period of time, e.g., daily for 21 day cycle cycle, or daily for 28 day cycle. In one embodiment, 1, 2, 3, 4, 5, 6, 7, 8, 9, 10, 11, 12 or more cycles of rapamycin are administered. In one embodiment, the kinase inhibitor is an mTOR inhibitor, e.g., everolimus and the everolimus is administered at a dose of about 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg (e.g., 10 mg) daily for a period of time, e.g., daily for 28 day cycle. In one embodiment, 1, 2, 3, 4, 5, 6, 7, 8, 9, 10, 11, 12 or more cycles of everolimus are administered.

In one embodiment, the kinase inhibitor is an MNK inhibitor selected from CGP052088; 4-amino-3-(p-fluorophenylamino)-pyrazolo [3,4-d] pyrimidine (CGP57380); cercosporamide; ETC-1780445-2; and 4-amino-5-(4-fluoroanilino)-pyrazolo [3,4-d] pyrimidine.

In one embodiment, the kinase inhibitor is a dual phosphatidylinositol 3-kinase (PI3K) and mTOR inhibitor selected from 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF-04691502); N-[4-[[4-(Dimethylamino)-1-piperidinyl]carbonyl]phenyl]-N′-[4-(4,6-di-4-morpholinyl-1,3,5-triazin-2-yl)phenyl]urea (PF-05212384, PKI-587); 2-Methyl-2-{4-[3-methyl-2-oxo-8-(quinolin-3-yl)-2,3-dihydro-1H-imidazo[4,5-c]quinolin-1-yl]phenyl}propanenitrile (BEZ-235); apitolisib (GDC-0980, RG7422); 2,4-Difluoro-N-{2-(methyloxy)-5-[4-(4-pyridazinyl)-6-quinolinyl]-3-pyridinyl}benzenesulfonamide (GSK2126458); 8-(6-methoxypyridin-3-yl)-3-methyl-1-(4-(piperazin-1-yl)-3-(trifluoromethyl)phenyl)-1H-imidazo[4,5-c]quinolin-2(3H)-one Maleic acid (NVP-BGT226); 3-[4-(4-Morpholinylpyrido[3′,2′:4,5]furo[3,2-d]pyrimidin-2-yl]phenol (PI-103); 5-(9-isopropyl-8-methyl-2-morpholino-9H-purin-6-yl)pyrimidin-2-amine (VS-5584, SB2343); and N-[2-[(3,5-Dimethoxyphenyl)amino]quinoxalin-3-yl]-4-[(4-methyl-3-methoxyphenyl)carbonyl]aminophenylsulfonamide (XL765).

In one embodiment, the kinase inhibitor is an MNK inhibitor selected from CGP052088; 4-amino-3-(p-fluorophenylamino)-pyrazolo [3,4-d] pyrimidine (CGP57380); cercosporamide; ETC-1780445-2; and 4-amino-5-(4-fluoroanilino)-pyrazolo [3,4-d] pyrimidine.

In embodiments, the combination described herein, e.g., a CAR-expressing cell (e.g., CD19 CAR-expressing cell) and a PD-1 inhibitor, is administered to a subject in combination with a phosphoinositide 3-kinase (PI3K) inhibitor (e.g., a PI3K inhibitor described herein, e.g., idelalisib or duvelisib) and/or rituximab. In embodiments, a CAR-expressing cell described herein is administered to a subject in combination with idelalisib and rituximab. In embodiments, a CAR-expressing cell described herein is administered to a subject in combination with duvelisib and rituximab. Idelalisib (also called GS-1101 or CAL-101; Gilead) is a small molecule that blocks the delta isoform of PI3K. Idelalisib has the chemical name: (5-Fluoro-3 -phenyl-2-[(1S)-1-(7H-purin-6-ylamino)propyl]-4(3H)-quinazolinone).

Duvelisib (also called IPI-145; Infinity Pharmaceuticals and Abbvie) is a small molecule that blocks PI3K-δ,γ. Duvelisib has the chemical name (8-Chloro-2-phenyl-3-[(1S)-1-(9H-purin-6-ylamino)ethyl]-1(2H)-isoquinolinone).

In embodiments, the subject has CLL. In embodiments, the subject has relapsed CLL, e.g., the subject has previously been administered a cancer therapy (e.g., previously been administered an anti-CD20 antibody or previously been administered ibrutinib). For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgV_{H}) gene. In other embodiments, the subject does not comprise a leukemic cell comprising a mutation in the immunoglobulin heavy-chain variable-region (IgV_{H}) gene. In embodiments, the subject has a deletion in the long arm of chromosome 11 (del(11q)). In other embodiments, the subject does not have a del(11q). In embodiments, idelalisib is administered at a dosage of about 100-400 mg (e.g., 100-125, 125-150, 150-175, 175-200, 200-225, 225-250, 250-275, 275-300, 325-350, 350-375, or 375-400 mg), e.g., BID. In embodiments, duvelisib is administered at a dosage of about 15-100 mg (e.g., about 15-25, 25-50, 50-75, or 75-100 mg), e.g., twice a day. In embodiments, rituximab is administered at a dosage of about 350-550 mg/m² (e.g., 350-375, 375-400, 400-425, 425-450, 450-475, or 475-500 mg/m²), e.g., intravenously.

In one embodiment, the kinase inhibitor is a dual phosphatidylinositol 3-kinase (PI3K) and mTOR inhibitor selected from 2-Amino-8-[trans-4-(2-hydroxyethoxy)cyclohexyl]-6-(6-methoxy-3-pyridinyl)-4-methyl-pyrido[2,3 -d]pyrimidin-7(8H)-one (PF-04691502); N-[4-[[4-(Dimethylamino)-1-piperidinyl]carbonyl]phenyl]-N′-[4-(4,6-di-4-morpholinyl-1,3,5-triazin-2-yl)phenyl]urea (PF-05212384, PKI-587); 2-Methyl-2-{4-[3-methyl-2-oxo-8-(quinolin-3-yl)-2,3-dihydro-1H-imidazo[4,5-c]quinolin-1-yl]phenyl}propanenitrile (BEZ-235); apitolisib (GDC-0980, RG7422); 2,4-Difluoro-N-{2-(methyloxy)-5-[4-(4-pyridazinyl)-6-quinolinyl]-3-pyridinyl}benzenesulfonamide (GSK2126458); 8-(6-methoxypyridin-3-yl)-3-methyl-1-(4-(piperazin-1-yl)-3-(trifluoromethyl)phenyl)-1H-imidazo[4,5-c]quinolin-2(3H)-one Maleic acid (NVP-BGT226); 3-[4-(4-Morpholinylpyrido[3′,2′:4,5]furo[3,2-d]pyrimidin-2-yl]phenol (PI-103); 5-(9-isopropyl-8-methyl-2-morpholino-9H-purin-6-yl)pyrimidin-2-amine (VS-5584, SB2343); and N-[2-[(3,5-Dimethoxyphenyl)amino]quinoxalin-3-yl]-4-[(4-methyl-3-methoxyphenyl)carbonyl]aminophenylsulfonamide (XL765).

In embodiments, a CAR-expressing cell described herein is administered to a subject in combination with an anaplastic lymphoma kinase (ALK) inhibitor. Exemplary ALK kinases include but are not limited to crizotinib (Pfizer), ceritinib (Novartis), alectinib (Chugai), brigatinib (also called AP26113; Ariad), entrectinib (Ignyta), PF-06463922 (Pfizer), TSR-011 (Tesaro) (see, e.g., Clinical Trial Identifier No. NCT02048488), CEP-37440 (Teva), and X-396 (Xcovery). In some embodiments, the subject has a solid cancer, e.g., a solid cancer described herein, e.g., lung cancer.

The chemical name of crizotinib is 3-[(1R)-1-(2,6-dichloro-3-fluorophenyl)ethoxy]-5-(1-piperidin-4-ylpyrazol-4-yl)pyridin-2-amine. The chemical name of ceritinib is 5-Chloro-N²-[2-isopropoxy-5-methyl-4-(4-piperidinyl)phenyl]-N⁴-[2-(isopropylsulfonyl)phenyl]-2,4-pyrimidinediamine. The chemical name of alectinib is 9-ethyl-6,6-dimethyl-8-(4-morpholinopiperidin-1-yl)-11-oxo-6,11-dihydro-5H-benzo [b]carbazole-3-carbonitrile. The chemical name of brigatinib is 5-Chloro-N²-{4-[4-(dimethylamino)-1-piperidinyl]-2-methoxyphenyl}-N⁴-[2-(dimethylphosphoryl)phenyl]-2,4-pyrimidinediamine. The chemical name of entrectinib is N-(5-(3,5-difluorobenzyl)-1H-indazol-3-yl)-4-(4-methylpiperazin-1-yl)-2-((tetrahydro-2H-pyran-4-yl)amino)benzamide. The chemical name of PF-06463922 is (10R)-7-Amino-12-fluoro-2,10,16-trimethyl-15-oxo-10,15,16,17-tetrahydro-2H-8,4-(metheno)pyrazolo[4,3-h][2,5,11]-benzoxadiazacyclotetradecine-3-carbonitrile. The chemical structure of CEP-37440 is (S)-2-((5-chloro-2-((6-(4-(2-hydroxyethyl)piperazin-1-yl)-1-methoxy-6,7,8,9-tetrahydro-5H-benzo[7]annulen-2-yl)amino)pyrimidin-4-yl)amino)-N-methylbenzamide. The chemical name of X-396 is (R)-6-amino-5-(1-(2,6-dichloro-3-fluorophenyl)ethoxy)-N-(4-(4-methylpiperazine-1-carbonyl)phenyl)pyridazine-3-carboxamide.

Drugs that inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993) can also be used. In a further aspect, the cell compositions of the present disclosure may be administered to a patient in conjunction with (e.g., before, simultaneously or following) bone marrow transplantation, T cell ablative therapy using chemotherapy agents such as, fludarabine, external-beam radiation therapy (XRT), cyclophosphamide, and/or antibodies such as OKT3 or CAMPATH. In one aspect, the cell compositions of the present disclosure are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present disclosure. In an additional embodiment, expanded cells are administered before or following surgery.

In embodiments, the combination described herein, e.g., a CAR-expressing cell (e.g., CD19 CAR-expressing cell) and a PD-1 inhibitor, is administered to a subject in combination with an indoleamine 2,3-dioxygenase (IDO) inhibitor. IDO is an enzyme that catalyzes the degradation of the amino acid, L-tryptophan, to kynurenine. Many cancers overexpress IDO, e.g., prostatic, colorectal, pancreatic, cervical, gastric, ovarian, head, and lung cancer. pDCs, macrophages, and dendritic cells (DCs) can express IDO. Without being bound by theory, it is thought that a decrease in L-tryptophan (e.g., catalyzed by IDO) results in an immunosuppressive milieu by inducing T-cell anergy and apoptosis. Thus, without being bound by theory, it is thought that an IDO inhibitor can enhance the efficacy of a CAR-expressing cell described herein, e.g., by decreasing the suppression or death of a CAR-expressing immune cell. In embodiments, the subject has a solid tumor, e.g., a solid tumor described herein, e.g., prostatic, colorectal, pancreatic, cervical, gastric, ovarian, head, or lung cancer. Exemplary inhibitors of IDO include but are not limited to 1-methyl-tryptophan, indoximod (NewLink Genetics) (see, e.g., Clinical Trial Identifier Nos. NCT01191216; NCT01792050), and INCB024360 (Incyte Corp.) (see, e.g., Clinical Trial Identifier Nos. NCT01604889; NCT01685255)

In embodiments, the combination described herein, e.g., a CAR-expressing cell (e.g., CD19 CAR-expressing cell) and a PD-1 inhibitor, is administered to a subject in combination with a modulator of myeloid-derived suppressor cells (MDSCs). MDSCs accumulate in the periphery and at the tumor site of many solid tumors. These cells suppress T cell responses, thereby hindering the efficacy of CAR-expressing cell therapy. Without being bound by theory, it is thought that administration of a MDSC modulator enhances the efficacy of a CAR-expressing cell described herein. In an embodiment, the subject has a solid tumor, e.g., a solid tumor described herein, e.g., glioblastoma. Exemplary modulators of MDSCs include but are not limited to MCS110 and BLZ945. MCS110 is a monoclonal antibody (mAb) against macrophage colony-stimulating factor (M-CSF). See, e.g., Clinical Trial Identifier No. NCT00757757. BLZ945 is a small molecule inhibitor of colony stimulating factor 1 receptor (CSF1R). See, e.g., Pyonteck et al. Nat. Med. 19(2013):1264-72. The structure of BLZ945 is shown below.

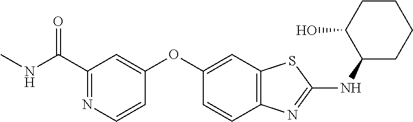 [SMILES: CNC(=O)c1cc(Oc2ccc3nc(N[C@@H]4CCCC[C@H]4O)sc3c2)ccn1]

In embodiments, the combination described herein, e.g., a CAR-expressing cell (e.g., CD19 CAR-expressing cell) and a PD-1 inhibitor, herein is administered to a subject in combination with an agent that inhibits or reduces the activity of immunosuppressive plasma cells. Immunosuppressive plasma cells have been shown to impede T cell-dependent immunogenic chemotherapy, such as oxaliplatin (Shalapour et al., Nature 2015, 521:94-101). In an embodiment, immunosuppressive plasma cells can express one or more of IgA, interleukin (IL)-10, and PD-L1. In an embodiment, the agent is a BCMA CAR-expressing cell.

In some embodiments , the combination described herein, e.g., a CAR-expressing cell (e.g., CD19 CAR-expressing cell) and a PD-1 inhibitor, is administered to a subject in combination with a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15 (Admune Therapeutics, LLC). hetIL-15 is a heterodimeric non-covalent complex of IL-15 and IL-15Ra. hetIL-15 is described in, e.g., U.S. Pat. No. 8,124,084, U.S. 2012/0177598, U.S. 2009/0082299, U.S. 2012/0141413, and U.S. 2011/0081311, incorporated herein by reference. In embodiments, het-IL-15 is administered subcutaneously. In embodiments, the subject has a cancer, e.g., solid cancer, e.g., melanoma or colon cancer. In embodiments, the subject has a metastatic cancer.

In embodiments, a subject having a disease described herein is administered a combination described herein, e.g., a CAR-expressing cell (e.g., CD19 CAR-expressing cell) and a PD-1 inhibitor, in combination with an agent, e.g., cytotoxic or chemotherapy agent, a biologic therapy (e.g., antibody, e.g., monoclonal antibody, or cellular therapy), or an inhibitor (e.g., kinase inhibitor). In embodiments, the subject is administered a CAR-expressing cell described herein in combination with a cytotoxic agent, e.g., CPX-351 (Celator Pharmaceuticals), cytarabine, daunorubicin, vosaroxin (Sunesis Pharmaceuticals), sapacitabine (Cyclacel Pharmaceuticals), idarubicin, or mitoxantrone. CPX-351 is a liposomal formulation comprising cytarabine and daunorubicin at a 5:1 molar ratio. In embodiments, the subject is administered a CAR-expressing cell described herein in combination with a hypomethylating agent, e.g., a DNA methyltransferase inhibitor, e.g., azacitidine or decitabine. In embodiments, the subject is administered a CAR-expressing cell described herein in combination with a biologic therapy, e.g., an antibody or cellular therapy, e.g., 225Ac-lintuzumab (Actimab-A; Actinium Pharmaceuticals), IPH2102 (Innate Pharma/Bristol Myers Squibb), SGN-CD33A (Seattle Genetics), or gemtuzumab ozogamicin (Mylotarg; Pfizer). SGN-CD33A is an antibody-drug conjugate (ADC) comprising a pyrrolobenzodiazepine dimer that is attached to an anti-CD33 antibody. Actimab-A is an anti-CD33 antibody (lintuzumab) labeled with actinium. IPH2102 is a monoclonal antibody that targets killer immunoglobulin-like receptors (KIRs). In embodiments, the subject is administered a CAR-expressing cell described herein in combination a FLT3 inhibitor, e.g., sorafenib (Bayer), midostaurin (Novartis), quizartinib (Daiichi Sankyo), crenolanib (Arog Pharmaceuticals), PLX3397 (Daiichi Sankyo), AKN-028 (Akinion Pharmaceuticals), or ASP2215 (Astellas). In embodiments, the subject is administered a CAR-expressing cell described herein in combination with an isocitrate dehydrogenase (IDH) inhibitor, e.g., AG-221 (Celgene/Agios) or AG-120 (Agios/Celgene). In embodiments, the subject is administered a CAR-expressing cell described herein in combination with a cell cycle regulator, e.g., inhibitor of polo-like kinase 1 (Plk1), e.g., volasertib (Boehringer Ingelheim); or an inhibitor of cyclin-dependent kinase 9 (Cdk9), e.g., alvocidib (Tolero Pharmaceuticals/Sanofi Aventis). In embodiments, the subject is administered a CAR-expressing cell described herein in combination with a B cell receptor signaling network inhibitor, e.g., an inihibitor of B-cell lymphoma 2 (Bc1-2), e.g., venetoclax (Abbvie/Roche); or an inhibitor of Bruton's tyrosine kinase (Btk), e.g., ibrutinib (Pharmacyclics/Johnson & Johnson Janssen Pharmaceutical). In embodiments, the subject is administered a CAR-expressing cell described herein in combination with an inhibitor of M1 aminopeptidase, e.g., tosedostat (CTI BioPharma/Vernalis); an inhibitor of histone deacetylase (HDAC), e.g., pracinostat (MEI Pharma); a multi-kinase inhibitor, e.g., rigosertib (Onconova Therapeutics/Baxter/SymBio); or a peptidic CXCR4 inverse agonist, e.g., BL-8040 (BioLineRx).

In another embodiment, the subjects receive an infusion of the CAR-expressing cell, e.g., compositions of the present disclosure prior to transplantation, e.g., allogeneic stem cell transplant, of cells. In a preferred embodiment, CAR expressing cells transiently express CAR, e.g., by electroporation of an mRNA encoding a CAR, whereby the expression of the CAR is terminated prior to infusion of donor stem cells to avoid engraftment failure.

Some patients may experience allergic reactions to the compounds of the present disclosure and/or other anti-cancer agent(s) during or after administration; therefore, anti-allergic agents are often administered to minimize the risk of an allergic reaction. Suitable anti-allergic agents include corticosteroids, such as dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

Some patients may experience nausea during and after administration of the compound of the present disclosure and/or other anti-cancer agent(s); therefore, anti-emetics are used in preventing nausea (upper stomach) and vomiting. Suitable anti-emetics include aprepitant (Emend®), ondansetron (Zofran®), granisetron HCl (Kytril®), lorazepam (Ativan®. dexamethasone (Decadron®), prochlorperazine (Compazine®), casopitant (Rezonic® and Zunrisa®), and combinations thereof.

Medication to alleviate the pain experienced during the treatment period is often prescribed to make the patient more comfortable. Common over-the-counter analgesics, such Tylenol®, are often used. However, opioid analgesic drugs such as hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®) are also useful for moderate or severe pain.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. Suitable cytoprotective agents include Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid). The structure of the active compounds identified by code numbers, generic or trade names may be taken from the actual edition of the standard compendium “The Merck Index” or from databases, e.g. Patents International (e.g. IMS World Publications).

The above-mentioned compounds, which can be used in combination with a compound of the present disclosure, can be prepared and administered as described in the art, such as in the documents cited above.

In one embodiment, the present disclosure provides pharmaceutical compositions comprising at least one compound of the present disclosure (e.g., a compound of the present disclosure) or a pharmaceutically acceptable salt thereof together with a pharmaceutically acceptable carrier suitable for administration to a human or animal subject, either alone or together with other anti-cancer agents.

In one embodiment, the present disclosure provides methods of treating human or animal subjects suffering from a cellular proliferative disease, such as cancer. The present disclosure provides methods of treating a human or animal subject in need of such treatment, comprising administering to the subject a therapeutically effective amount of a compound of the present disclosure (e.g., a compound of the present disclosure) or a pharmaceutically acceptable salt thereof, either alone or in combination with other anti-cancer agents.

In particular, compositions will either be formulated together as a combination therapeutic or administered separately.

In combination therapy, the compound of the present disclosure and other anti-cancer agent(s) may be administered either simultaneously, concurrently or sequentially with no specific time limits, wherein such administration provides therapeutically effective levels of the two compounds in the body of the patient.

In a preferred embodiment, the compound of the present disclosure and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present disclosure and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect of the present disclosure, kits that include one or more compound of the present disclosure and a combination partner as disclosed herein are provided. Representative kits include (a) a compound of the present disclosure or a pharmaceutically acceptable salt thereof, (b) at least one combination partner, e.g., as indicated above, whereby such kit may comprise a package insert or other labeling including directions for administration.

A compound of the present disclosure may also be used to advantage in combination with known therapeutic processes, for example, the administration of hormones or especially radiation. A compound of the present disclosure may in particular be used as a radiosensitizer, especially for the treatment of tumors which exhibit poor sensitivity to radiotherapy. In one embodiment, the subject can be administered an agent which reduces or ameliorates a side effect associated with the administration of a CAR-expressing cell. Side effects associated with the administration of a CAR-expressing cell include, but are not limited to CRS, and hemophagocytic lymphohistiocytosis (HLH), also termed Macrophage Activation Syndrome (MAS).

Accordingly, the methods described herein can comprise administering a CAR-expressing cell described herein to a subject and further administering one or more agents to manage elevated levels of a soluble factor resulting from treatment with a CAR-expressing cell. In one embodiment, the soluble factor elevated in the subject is one or more of IFN-γ, TNFα, IL-2 and IL-6. In an embodiment, the factor elevated in the subject is one or more of IL-1, GM-CSF, IL-10, IL-8, IL-5 and fraktalkine. Therefore, an agent administered to treat this side effect can be an agent that neutralizes one or more of these soluble factors. In one embodiment, the agent that neutralizes one or more of these soluble forms is an antibody or antigen binding fragment thereof. Examples of such agents include, but are not limited to a steroid (e.g., corticosteroid), an inhibitor of TNFα, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept. Small molecule inhibitor of TNFα include, but are not limited to, xanthine derivatives (e.g. pentoxifylline) and bupropion. An example of an IL-6 inhibitor is an anti-IL-6 antibody molecule such as tocilizumab (toc), sarilumab, elsilimomab, CNTO 328, ALD518/BMS-945429, CNTO 136, CPSI-2364, CDP6038, VX30, ARGX-109, FE301, and FM101. In one embodiment, the anti-IL-6 antibody molecule is tocilizumab. An example of an IL-1R based inhibitor is anakinra.

In some embodiment, the subject is administered a corticosteroid, such as, e.g., methylprednisolone, hydrocortisone, among others.

In some embodiments, the subject is administered a vasopressor, such as, e.g., norepinephrine, dopamine, phenylephrine, epinephrine, vasopressin, or a combination thereof.

In an embodiment, the subject can be administered an antipyretic agent. In an embodiment, the subject can be administered an analgesic agent.

In one embodiment, the subject can be further administered an agent which enhances the activity or fitness of a CAR-expressing cell. For example, in one embodiment, the agent can be an agent which inhibits a molecule that modulates or regulates, e.g., inhibits, T cell function. In some embodiments, the molecule that modulates or regulates T cell function is an inhibitory molecule. Inhibitory molecules, e.g., Programmed Death 1 (PD-1) or PD-1 ligand (PD-L1), can, in some embodiments, decrease the ability of a CAR-expressing cell to mount an immune effector response. Examples of inhibitory molecules include PD-1, PD-L1, CTLA4, TIM3, CEACAM (e.g., CEACAM-1, CEACAM-3 and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF beta. Inhibition of a molecule that modulates or regulates, e.g., inhibits, T cell function, e.g., by inhibition at the DNA, RNA or protein level, can optimize a CAR-expressing cell performance. In embodiments, an agent, e.g., an inhibitory nucleic acid, e.g., an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used to inhibit expression of an inhibitory molecule in the CAR-expressing cell. In an embodiment, the inhibitor is an shRNA.

In an embodiment, the agent that modulates or regulates, e.g., inhibits, T-cell function is inhibited within a CAR-expressing cell. In these embodiments, a dsRNA molecule that inhibits expression of a molecule that modulates or regulates, e.g., inhibits, T-cell function is linked to the nucleic acid that encodes a component, e.g., all of the components, of the CAR. In an embodiment, a nucleic acid molecule that encodes a dsRNA molecule that inhibits expression of the molecule that modulates or regulates, e.g., inhibits, T-cell function is operably linked to a promoter, e.g., a H1- or a U6-derived promoter such that the dsRNA molecule that inhibits expression of the molecule that modulates or regulates, e.g., inhibits, T-cell function is expressed, e.g., is expressed within a CAR-expressing cell. See e.g., Tiscornia G., “Development of Lentiviral Vectors Expressing siRNA,” Chapter 3, in Gene Transfer: Delivery and Expression of DNA and RNA (eds. Friedmann and Rossi). Cold Spring Harbor Laboratory Press, Cold Spring Harbor, N.Y., USA, 2007; Brummelkamp T R, et al. (2002) Science 296: 550-553; Miyagishi M, et al. (2002) Nat. Biotechnol. 19: 497-500. In an embodiment the nucleic acid molecule that encodes a dsRNA molecule that inhibits expression of the molecule that modulates or regulates, e.g., inhibits, T-cell function is present on the same vector, e.g., a lentiviral vector, that comprises a nucleic acid molecule that encodes a component, e.g., all of the components, of the CAR. In such an embodiment, the nucleic acid molecule that encodes a dsRNA molecule that inhibits expression of the molecule that modulates or regulates, e.g., inhibits, T-cell function is located on the vector, e.g., the lentiviral vector, 5′- or 3′- to the nucleic acid that encodes a component, e.g., all of the components, of the CAR. The nucleic acid molecule that encodes a dsRNA molecule that inhibits expression of the molecule that modulates or regulates, e.g., inhibits, T-cell function can be transcribed in the same or different direction as the nucleic acid that encodes a component, e.g., all of the components, of the CAR. In an embodiment the nucleic acid molecule that encodes a dsRNA molecule that inhibits expression of the molecule that modulates or regulates, e.g., inhibits, T-cell function is present on a vector other than the vector that comprises a nucleic acid molecule that encodes a component, e.g., all of the components, of the CAR. In an embodiment, the nucleic acid molecule that encodes a dsRNA molecule that inhibits expression of the molecule that modulates or regulates, e.g., inhibits, T-cell function it transiently expressed within a CAR-expressing cell. In an embodiment, the nucleic acid molecule that encodes a dsRNA molecule that inhibits expression of the molecule that modulates or regulates, e.g., inhibits, T-cell function is stably integrated into the genome of a CAR-expressing cell. Configurations of exemplary vectors for expressing a component, e.g., all of the components, of the CAR with a dsRNA molecule that inhibits expression of the molecule that modulates or regulates, e.g., inhibits, T-cell function, is provided, e.g., in FIG. 47 of International Publication WO2015/090230, filed Dec. 19, 2014, which is herein incorporated by reference.

Combination Therapies with Inhibitors of Checkpoint Molecules

In one embodiment, the agent that modulates or regulates, e.g., inhibits, T-cell function can be, e.g., an antibody or antibody fragment that binds to an inhibitory molecule. For example, the agent can be an antibody or antibody fragment that binds to PD-1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206).). In an embodiment, the agent is an antibody or antibody fragment that binds to TIM3. In an embodiment, the agent is an antibody or antibody fragment that binds to LAG3. In an embodiment, the agent is an antibody or antibody fragment that binds to PD-L1.

PD-1 is described in greater detail above. Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1. The term “Programmed Death Ligand 1” or “PD-L1” include isoforms, mammalian, e.g., human PD-L1, species homologs of human PD-1, and analogs comprising at least one common epitope with PD-L1. The amino acid sequence of PD-L1, e.g., human PD-1, is known in the art, e.g., Dong et al. (1999) Nat Med. 5(12):1365-9; Freeman et al. (2000) J Exp Med. 192(7):1027-34).

Antibodies, antibody fragments, and other inhibitors (e.g., small molecule; polypeptide, e.g., a fusion protein; or inhibitory nucleic acid; e.g., a siRNA or shRNA inhibitors), e.g., of PD-L1 and PD-L2 are available in the art and may be used combination with a CAR (e.g., CD19 CAR) (e.g., and a PD-1 inhibitor) described herein. MEDI4736 (Medimmune) is a human monoclonal antibody that binds to PDL1, and inhibits interaction of the ligand with PD1.

In one embodiment, the anti-PD-L1 antibody is an anti-PD-L1 antibody molecule as disclosed in in US 2016/0108123, published on Apr. 21, 2016, entitled “Antibody Molecules to PD-L1 and Uses Thereof,” incorporated by reference in its entirety.

In some embodiments, the anti-PD-L1 antibody is MSB0010718C. MSB0010718C (also referred to as A09-246-2; Merck Serono or avelumab) is a monoclonal antibody that binds to PD-L1. Exemplary humanized anti-PD-L1 antibodies are disclosed in WO2013/079174 (incorporated herein by reference), and having a sequence disclosed herein (or a sequence substantially identical or similar thereto, e.g., a sequence at least 85%, 90%, 95% identical or higher to the sequence specified).

MDPL3280A (Genentech/Roche) is a human Fc optimized IgG1 monoclonal antibody that binds to PD-L1. MDPL3280A, also known as Atezolizumab, and other human monoclonal antibodies to PD-L1 are disclosed in U.S. Pat. No. 7,943,743 and U.S Publication No.: 20120039906, incorporated herein by reference.

In one embodiment, the anti-PD-L1 antibody molecule comprises one or more of the CDR sequences (or collectively all of the CDR sequences), the heavy chain or light chain variable region sequence, or the heavy chain or light chain sequence of Atezolizumab. In embodiments, Atezolizumab is administered in combination with a CAR

In an embodiment, a CAR therapy, e.g., a CAR-expressing cell (e.g., CD19 CAR-expressing cell) can be used in combination with an anti-PDL1 antibody (e.g., Atezolizumab) for treating a subject with a lymphoma, e.g., DLBCL. In some embodiments, the subject has DLBCL, e.g., r/r DLBCL, and has had prior anti-CD20 and anthracycline therapy. In some embodiments, Atezolizumab can be administered concurrently with, before or after administration of a CAR therapy (e.g., a CD19 CAR-expressing cell). In some embodiments, Atezolizumab is administered concurrently with a CAR therapy (e.g., a CD19 CAR-expressing cell). In some embodiments, Atezolizumab is administered at least one time (e.g., one, two, three, four, five, six or more times) at a dose of 1200 mg (e.g., 1000, 1200, 1500 or 2000 mg) every 3 weeks. In some embodiments, Atezolizumab is administered four times at a dose of 1200 mg every 3 weeks.

Other anti-PD-L1 binding agents include YW243.55.S70 (heavy and light chain variable regions are shown in SEQ ID NOs: 20 and 21 in WO2010/077634) and MDX-1105 (also referred to as BMS-936559, and, e.g., anti-PD-L1 binding agents disclosed in WO2007/005874). AMP-224 (B7-DCIg; Amplimmune; e.g., disclosed in WO2010/027827 and WO2011/066342), is a PD-L2 Fc fusion soluble receptor that blocks the interaction between PD1 and B7-H1. Examples of RNAi agents include long dsRNA, siRNA, shRNA, and microRNAs. Inhibitory nucleic acids described herein include, but are not limited to, an aptamer, a morpholino, a ribozyme, and a nucleic acid sequence, e.g., plasmids or vectors, that comprise or encode a long dsRNA, siRNA, shRNA, or microRNA.

TIM3 (T cell immunoglobulin-3) also negatively regulates T cell function, particularly in IFN-g-secreting CD4+ T helper 1 and CD8+ T cytotoxic 1 cells, and plays a critical role in T cell exhaustion. Inhibition of the interaction between TIM3 and its ligands, e.g., galectin-9 (Gal9), phosphotidylserine (PS), and HMGB1, can increase immune response. Antibodies, antibody fragments, and other inhibitors of TIM3 and its ligands are available in the art and may be used combination with a CAR (e.g., CD19 CAR) described herein. For example, antibodies, antibody fragments, small molecules, or peptide inhibitors that target TIM3 binds to the IgV domain of TIM3 to inhibit interaction with its ligands. Antibodies and peptides that inhibit TIM3 are disclosed in WO2013/006490 and US20100247521. Other anti-TIM3 antibodies include humanized versions of RMT3-23 (disclosed in Ngiow et al., 2011, Cancer Res, 71:3540-3551), and clone 8B.2C12 (disclosed in Monney et al., 2002, Nature, 415:536-541). Bi-specific antibodies that inhibit TIM3 and PD-1 are disclosed in US20130156774.

In one embodiment, the anti-TIM3 antibody or fragment thereof is an anti-TIM3 antibody molecule as described in US 2015/0218274, entitled “Antibody Molecules to TIM3 and Uses Thereof,” incorporated by reference in its entirety. In one embodiment, the anti-TIM3 antibody molecule includes at least one, two, three, four, five or six CDRs (or collectively all of the CDRs) from a heavy and light chain variable region from an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences, or closely related CDRs, e.g., CDRs which are identical or which have at least one amino acid alteration, but not more than two, three or four alterations (e.g., substitutions, deletions, or insertions, e.g., conservative substitutions).

In yet another embodiment, the anti-TIM3 antibody molecule comprises at least one, two, three or four variable regions from an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences.

In other embodiments, the agent which enhances the activity of a CAR-expressing cell is a CEACAM inhibitor (e.g., CEACAM-1, CEACAM-3, and/or CEACAM-5 inhibitor). In one embodiment, the inhibitor of CEACAM is an anti-CEACAM antibody molecule. Exemplary anti-CEACAM-1 antibodies are described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep 2; 5(9). pii: e12529 (DOI:10:1371/journal.pone.0021146), or crossreacts with CEACAM-1 and CEACAM-5 as described in, e.g., WO 2013/054331 and US 2014/0271618.

Without wishing to be bound by theory, carcinoembryonic antigen cell adhesion molecules (CEACAM), such as CEACAM-1 and CEACAM-5, are believed to mediate, at least in part, inhibition of an anti-tumor immune response (see e.g., Markel et al. J Immunol. 2002 Mar. 15; 168(6):2803-10; Markel et al. J Immunol. 2006 Nov. 1; 177(9):6062-71; Markel et al. Immunology. 2009 February; 126(2):186-200; Markel et al. Cancer Immunol Immunother. 2010 February; 59(2):215-30; Ortenberg et al. Mol Cancer Ther. 2012 June; 11(6):1300-10; Stern et al. J Immunol. 2005 Jun. 1; 174(11):6692-701; Zheng et al. PLoS One. 2010 Sep. 2; 5(9). pii: e12529). For example, CEACAM-1 has been described as a heterophilic ligand for TIM-3 and as playing a role in TIM-3-mediated T cell tolerance and exhaustion (see e.g., WO 2014/022332; Huang, et al. (2014) Nature doi:10.1038/nature13848). In embodiments, co-blockade of CEACAM-1 and TIM-3 has been shown to enhance an anti-tumor immune response in xenograft colorectal cancer models (see e.g., WO 2014/022332; Huang, et al. (2014), supra). In other embodiments, co-blockade of CEACAM-1 and PD-1 reduce T cell tolerance as described, e.g., in WO 2014/059251. Thus, CEACAM inhibitors can be used with the other immunomodulators described herein (e.g., anti-PD-1 and/or anti-TIM-3 inhibitors) to enhance an immune response against a cancer, e.g., a melanoma, a lung cancer (e.g., NSCLC), a bladder cancer, a colon cancer an ovarian cancer, and other cancers as described herein.

LAG3 (lymphocyte activation gene-3 or CD223) is a cell surface molecule expressed on activated T cells and B cells that has been shown to play a role in CD8+ T cell exhaustion. Antibodies, antibody fragments, and other inhibitors of LAG3 and its ligands are available in the art and may be used combination with a CAR (e.g., CD19 CAR) described herein. For example, BMS-986016 (Bristol-Myers Squib) is a monoclonal antibody that targets LAG3. IMP701 (Immutep) is an antagonist LAG3 antibody and IMP731 (Immutep and GlaxoSmithKline) is a depleting LAG3 antibody. Other LAG3 inhibitors include IMP321 (Immutep), which is a recombinant fusion protein of a soluble portion of LAG3 and Ig that binds to MHC class II molecules and activates antigen presenting cells (APC). Other antibodies are disclosed, e.g., in WO2010/019570.

In one embodiment, the anti-LAG3 antibody or fragment thereof is an anti-LAG3 antibody molecule as described in US 2015/0259420, entitled “Antibody Molecules to LAG3 and Uses Thereof,” incorporated by reference in its entirety. In one embodiment, the anti-LAG3 antibody molecule includes at least one, two, three, four, five or six CDRs (or collectively all of the CDRs) from a heavy and light chain variable region from an antibody chosen from any of BAP050-hum01, BAP050-hum02, BAP050-hum03, BAP050-hum04, BAP050-hum05, BAP050-hum06, BAP050-hum07, BAP050-hum08, BAP050-hum09, BAP050-hum10, BAP050-hum11, BAP050-hum12, BAP050-hum13, BAP050-hum14, BAP050-hum15, BAP050-hum16, BAP050-hum17, BAP050-hum18, BAP050-hum19, BAP050-hum20, huBAP050(Ser) (e.g., BAP050-hum01-Ser, BAP050-hum02-Ser, BAP050-hum03-Ser, BAP050-hum04-Ser, BAP050-hum05-Ser, BAP050-hum06-Ser, BAP050-hum07-Ser, BAP050-hum08-Ser, BAP050-hum09-Ser, BAP050-hum10-Ser, BAP050-hum11-Ser, BAP050-hum12-Ser, BAP050-hum13-Ser, BAP050-hum14-Ser, BAP050-hum15-Ser, BAP050-hum18-Ser, BAP050-hum19-Ser, or BAP050-hum20-Ser), BAP050-Clone-F, BAP050-Clone-G, BAP050-Clone-H, BAP050-Clone-I, or BAP050-Clone-J; or as described in Table 1 of US 2015/0259420; or encoded by the nucleotide sequence in Table 1; or a sequence substantially identical (e.g., at least 80%, 85%, 90%, 92%, 95%, 97%, 98%, 99% or higher identical) to any of the aforesaid sequences, or closely related CDRs, e.g., CDRs which are identical or which have at least one amino acid alteration, but not more than two, three or four alterations (e.g., substitutions, deletions, or insertions, e.g., conservative substitutions).

In yet another embodiment, the anti-LAG3 antibody molecule comprises at least one, two, three or four variable regions from an antibody described herein, e.g., an antibody chosen from any of BAP050-hum01, BAP050-hum02, BAP050-hum03, BAP050-hum04, BAP050-hum05, BAP050-hum06, BAP050-hum07, BAP050-hum08, BAP050-hum09, BAP050-hum10, BAP050-hum11, BAP050-hum12, BAP050-hum13, BAP050-hum14, BAP050-hum15, BAP050-hum16, BAP050-hum17, BAP050-hum18, BAP050-hum19, BAP050-hum20, huBAP050(Ser) (e.g., BAP050-hum01-Ser, BAP050-hum02-Ser, BAP050-hum03-Ser, BAP050-hum04-Ser, BAP050-hum05-Ser, BAP050-hum06-Ser, BAP050-hum07-Ser, BAP050-hum08-Ser, BAP050-hum09-Ser, BAP050-hum10-Ser, BAP050-hum11-Ser, BAP050-hum12-Ser, BAP050-hum13-Ser, BAP050-hum14-Ser, BAP050-hum15-Ser, BAP050-hum18-Ser, BAP050-hum19-Ser, or BAP050-hum20-Ser), BAP050-Clone-F, BAP050-Clone-G, BAP050-Clone-H, BAP050-Clone-I, or BAP050-Clone-J; or as described in Table 1 of US 2015/0259420; or encoded by the nucleotide sequence in Tables 1; or a sequence substantially identical (e.g., at least 80%, 85%, 90%, 92%, 95%, 97%, 98%, 99% or higher identical) to any of the aforesaid sequences.

In some embodiments, the agent which enhances the activity of a CAR-expressing cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the CAR. In another embodiment, the fusion protein is expressed by a cell, e.g., a T cell that does not express a CAR of the present disclosure.

In embodiments, the subject is administered an additional agent (in further combination with a CAR-expressing cell and a PD-1 inhibitor described herein), where the additional agent is an inhibitor of an inhibitory molecule, e.g., checkpoint molecule, e.g., PD-1, PD-L1, CTLA4, TIM3, CEACAM (e.g., CEACAM-1, CEACAM-3 and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, or TGF beta. In embodiments, the additional agent is an inhibitor of PD-L1, e.g., FAZ053 (a hIgG4 humanized anti-PD-L1 monoclonal antibody), MPDL3280A, durvalumab (DEMI-4736), avelumab (MSB-0010718C), or BMS-936559. In embodiments, the additional agent is an additional inhibitor of PD-1, e.g., pembrolizumab, nivolumab, PDR001, MEDI-0680 (AMP-514), AMP-224, REGN-2810, or BGB-A317. In embodiments, the additional agent is an inhibitor of CTLA-4, e.g., ipilimumab. In embodiments, the additional agent is an inhibitor of LAG-3, e.g., LAG525 (a hIgG4 humanized anti-LAG-3 monoclonal antibody). In embodiments, the additional agent is an inhibitor of TIM-3, e.g., MBG453 (a hIgG4 humanized anti-TIM-3 monoclonal antibody). In embodiments, the additional agent is an inhibitor of the enzyme, B-Raf, e.g., dabrafenib (GSK2118436; N-{3-[5-(2-aminopyrimidin-4-yl)-2-tert-butyl-1,3-thiazol-4-yl]-2-fluorophenyl}-2,6-difluorobenzenesulfonamide). In embodiments, the additional agent is an inhibitor of MEK1 and/or MEK2, e.g., trametinib (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl}phenyl)acetamide). In embodiments, the additional agent comprises dabrafenib and trametinib. In embodiments, the additional agent is an inhibitor of GITR, e.g., GWN323. In embodiments, the additional agent is an agonist of STING (Stimulator of Interferon Genes), e.g., MIW815. In embodiments, the additional agent is an IL-15 agonist, e.g., NIZ985. In embodiments, the additional agent an inhibitor of adenosine receptor, e.g., NIR178. In embodiments, the additional agent is an inhibitor of macrophage colony stimulating factor (CSF-1), e.g., MCS110. In embodiments, the additional agent is an inhibitor of cMet, e.g., INC280. In embodiments, the additional agent is an inhibitor of porcupine (PORCN), e.g., WNT974. In embodiments, the additional agent is a histone deacetylase inhibitor, e.g., panobinost. In embodiments, the additional agent is an mTOR inhibitor, e.g., everolimus. In embodiments, the additional agent is a second mitochondrial-derived activator of caspases (SMAC) mimetic and/or an inhibitor of IAP (inhibiotor of apoptosis protein) family of proteins, e.g., LCL161. In embodiments, the additional agent is an inhibitor epidermal growth factor receptor (EGFR), e.g., EGF816. In embodiments, the additional agent is an inhibitor of IL-17, e.g., CJM112. In embodiments, the additional agent is an inhibitor of IL-lbeta, e.g., ILARIS.

In one embodiment, the agent which enhances activity of a CAR-expressing cell described herein is miR-17-92.

In one embodiment, the agent which enhances activity of a CAR-described herein is a cytokine. Cytokines have important functions related to T cell expansion, differentiation, survival, and homeostatis. Cytokines that can be administered to the subject receiving a CAR-expressing cell described herein include: IL-2, IL-4, IL-7, IL-9, IL-15, IL-18, and IL-21, or a combination thereof. In preferred embodiments, the cytokine administered is IL-7, IL-15, or IL-21, or a combination thereof. The cytokine can be administered once a day or more than once a day, e.g., twice a day, three times a day, or four times a day. The cytokine can be administered for more than one day, e.g. the cytokine is administered for 2 days, 3 days, 4 days, 5 days, 6 days, 1 week, 2 weeks, 3 weeks, or 4 weeks. For example, the cytokine is administered once a day for 7 days.

In embodiments, the cytokine is administered in combination with the combination described herein, e.g., a CAR-expressing cell (e.g., CD19 CAR-expressing cell) and a PD-1 inhibitor. The cytokine can be administered simultaneously or concurrently with the CAR-expressing cells, e.g., administered on the same day. The cytokine may be prepared in the same pharmaceutical composition as the CAR-expressing cells, or may be prepared in a separate pharmaceutical composition. Alternatively, the cytokine can be administered shortly after administration of the CAR-expressing cells, e.g., 1 day, 2 days, 3 days, 4 days, 5 days, 6 days, or 7 days after administration of the CAR-expressing T cells. In embodiments where the cytokine is administered in a dosing regimen that occurs over more than one day, the first day of the cytokine dosing regimen can be on the same day as administration with the CAR-expressing cells, or the first day of the cytokine dosing regimen can be 1 day, 2 days, 3 days, 4 days, 5 days, 6 days, or 7 days after administration of the CAR-expressing cells. In one embodiment, on the first day, the CAR-expressing cells are administered to the subject, and on the second day, a cytokine is administered once a day for the next 7 days. In a preferred embodiment, the cytokine to be administered in combination with CAR-expressing cells is IL-7, IL-15, or IL-21.

In other embodiments, the cytokine is administered a period of time after administration of CAR-expressing cells, e.g., at least 2 weeks, 3 weeks, 4 weeks, 6 weeks, 8 weeks, 10 weeks, 12 weeks, 4 months, 5 months, 6 months, 7 months, 8 months, 9 months, 10 months, 11 months, or 1 year or more after administration of CAR-expressing cells. In one embodiment, the cytokine is administered after assessment of the subject's response to the CAR-expressing cells. For example, the subject is administered CAR-expressing cells according to the dosage and regimens described herein. The response of the subject to CART therapy is assessed at 2 weeks, 3 weeks, 4 weeks, 6 weeks, 8 weeks, 10 weeks, 12 weeks, 4 months, 5 months, 6 months, 7 months, 8 months, 9 months, 10 months, 11 months, or 1 year or more after administration of CAR-expressing cells, using any of the methods described herein, including inhibition of tumor growth, reduction of circulating tumor cells, or tumor regression. Subjects that do not exhibit a sufficient response to CAR-expressing cell therapy can be administered a cytokine. Administration of the cytokine to the subject that has sub-optimal response to the CAR-expressing cell therapy improves CAR-expressing cell efficacy or anti-tumor activity. In a preferred embodiment, the cytokine administered after administration of CAR-expressing cells is IL-7.

Combination with a Low Dose of an mTOR Inhibitor

In one embodiment, the combination described herein, e.g., a CAR-expressing cell (e.g., CD19 CAR-expressing cell) and a PD-1 inhibitor, is administered in combination with a low, immune enhancing dose of an mTOR inhibitor.

In another embodiment, administration of a low, immune enhancing, dose of an mTOR inhibitor results in increased or prolonged proliferation of CAR-expressing cells, e.g., in culture or in a subject, e.g., as compared to non-treated CAR-expressing cells or a non-treated subject. In embodiments, increased proliferation is associated with in an increase in the number of CAR-expressing cells. Methods for measuring increased or prolonged proliferation are described in the Examples herein. In another embodiment, administration of a low, immune enhancing, dose of an mTOR inhibitor results in increased killing of cancer cells by CAR-expressing cells, e.g., in culture or in a subject, e.g., as compared to non-treated CAR-expressing cells or a non-treated subject. In embodiments, increased killing of cancer cells is associated with in a decrease in tumor volume.

In one embodiment, the cells expressing a CAR molecule, e.g., a CAR molecule described herein, are administered in combination with a low, immune enhancing dose of an mTOR inhibitor, e.g., an allosteric mTOR inhibitor, e.g., RAD001, or a catalytic mTOR inhibitor. For example, administration of the low, immune enhancing, dose of the mTOR inhibitor can be initiated prior to administration of a CAR-expressing cell described herein; completed prior to administration of a CAR-expressing cell described herein; initiated at the same time as administration of a CAR-expressing cell described herein; overlapping with administration of a CAR-expressing cell described herein; or continuing after administration of a CAR-expressing cell described herein.

Alternatively or in addition, administration of a low, immune enhancing, dose of an mTOR inhibitor can optimize immune effector cells to be engineered to express a CAR molecule described herein. In such embodiments, administration of a low, immune enhancing, dose of an mTOR inhibitor, e.g., an allosteric inhibitor, e.g., RAD001, or a catalytic inhibitor, is initiated or completed prior to harvest of immune effector cells, e.g., T cells or NK cells, to be engineered to express a CAR molecule described herein, from a subject.

In another embodiment, immune effector cells, e.g., T cells or NK cells, to be engineered to express a CAR molecule described herein, e.g., after harvest from a subject, or CAR-expressing immune effector cells, e.g., T cells or NK cells, e.g., prior to administration to a subject, can be cultured in the presence of a low, immune enhancing, dose of an mTOR inhibitor.

As used herein, the term “mTOR inhibitor” refers to a compound or ligand, or a pharmaceutically acceptable salt thereof, which inhibits the mTOR kinase in a cell. In an embodiment an mTOR inhibitor is an allosteric inhibitor. In an embodiment an mTOR inhibitor is a catalytic inhibitor.

Allosteric mTOR inhibitors include the neutral tricyclic compound rapamycin (sirolimus), rapamycin-related compounds, that is compounds having structural and functional similarity to rapamycin including, e.g., rapamycin derivatives, rapamycin analogs (also referred to as rapalogs) and other macrolide compounds that inhibit mTOR activity.

Rapamycin is a known macrolide antibiotic produced by Streptomyces hygroscopicus. Other suitable rapamycin analogs include, but are not limited to, RAD001, otherwise known as everolimus (Afinitor®), has the chemical name (1R,9S,12S,15R,16E,18R,19R,21R,23S,24E,26E,28E,30S,32S,35R)-1,18-dihydroxy-12-{(1R)-2-[(1S,3R,4R)-4-(2-hydroxyethoxy)-3-methoxycyclohexyl]-1-methylethyl}-19,30-dimethoxy-15,17,21,23,29,35-hexamethyl-11,36-dioxa-4-aza-tricyclo[30.3.1.04,9]hexatriaconta-16,24,26,28-tetraene-2,3,10,14,20-pentaone,sirolimus (rapamycin, AY-22989), 40-[3-hydroxy-2-(hydroxymethyl)-2-methylpropanoate]-rapamycin (also called temsirolimus or CCI-779) and ridaforolimus (AP-23573/MK-8669).b Other examples of allosteric mTor inhibtors include zotarolimus (ABT578) and umirolimus as described in US2005/0101624 the contents of which are incorporated by reference. Other suitable mTOR inhibitors are described in paragraphs 946 to 964 of International Publication WO2015/142675, filed March 13, 2015, which is incorporated by reference in its entirety. Low, immune enhancing doses of an mTOR inhibitor, suitable levels of mTOR inhibition associated with low doses of an mTOR inhibitor, methods for detecting the level of mTOR inhibition, and suitable pharmaceutical compositions thereof are further described in paragraphs 936 to 945 and 965 to 1003 of International Publication WO2015/142675, filed March 13, 2015, which is incorporated by reference in its entirety.

### Cytokine Release Syndrome (CRS)

Cytokine release syndrome (CRS) is a potentially life-threatening cytokine-associated toxicity that can occur as a result of cancer immunotherapy, e.g., cancer antibody therapies or T cell immunotherapies (e.g., CAR T cells). CRS results from high-level immune activation when large numbers of lymphocytes and/or myeloid cells release inflammatory cytokines upon activation. The severity of CRS and the timing of onset of symptoms can vary depending on the magnitude of immune cell activation, the type of therapy administered, and/or the extent of tumor burden in a subject. In the case of T-cell therapy for cancer, symptom onset is typically days to weeks after administration of the T-cell therapy, e.g., when there is peak in vivo T-cell expansion. See, e.g., Lee et al. Blood. 124.2(2014): 188-95.

Symptoms of CRS can include neurologic toxicity, disseminated intravascular coagulation, cardiac dysfunction, adult respiratory distress syndrome, renal failure, and/or hepatic failure. For example, symptoms of CRS include high fevers, nausea, transient hypotension, hypoxia, and the like. CRS may include clinical constitutional signs and symptoms such as fever, fatigue, anorexia, myalgias, arthalgias, nausea, vomiting, and headache. CRS may include clinical skin signs and symptoms such as rash. CRS may include clinical gastrointestinal signs and symsptoms such as nausea, vomiting and diarrhea. CRS may include clinical respiratory signs and symptoms such as tachypnea and hypoxemia. CRS may include clinical cardiovascular signs and symptoms such as tachycardia, widened pulse pressure, hypotension, increased cardac output (early) and potentially diminished cardiac output (late). CRS may include clinical coagulation signs and symptoms such as elevated d-dimer, hypofibrinogenemia with or without bleeding. CRS may include clinical renal signs and symptoms such as azotemia. CRS may include clinical hepatic signs and symptoms such as transaminitis and hyperbilirubinemia. CRS may include clinical neurologic signs and symptoms such as headache, mental status changes, confusion, delirium, word finding difficulty or frank aphasia, hallucinations, tremor, dymetria, altered gait, and seizures.

IL-6 is thought to be a mediator of CRS toxicity. See, e.g., id. High IL-6 levels may initiate a proinflammatory IL-6 signaling cascade, leading to one or more of the CRS symptoms. In some cases, the level of C-reactive protein (CRP) (a biomolecule produced by the liver, e.g., in response to IL-6) can be a measure of IL-6 activity. In some cases, CRP levels may increase several fold (e.g., several logs) during CRS. CRP levels can be measured using methods described herein, and/or standard methods available in the art.

### CRS Grading

In some embodiments, CRS can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. For example, criteria for grading CRS is provided herein as Table A. Unless otherwise specified, CRS as used herein refers to CRS according to the criteria of Table A.

TABLE A
CRS grading

| | |
|---|---|
| Gr1 | Supportive care only |
| Gr2 | IV therapies +/− hospitalization. |
| Gr3 | Hypotension requiring IV fluids or low-dose vasoactives or |
| | hypoxemia requiring oxygen, CPAP, or BIPAP. |
| Gr4 | Hypotension requiring high-dose vasoactives or hypoxemia |
| | requiring mechanical ventilation. |
| Gr 5 | Death |

### CRS Therapies

Therapies for CRS include IL-6 inhibitor or IL-6 receptor (IL-6R) inhibitors (e.g., tocilizumab or siltuximab), sgp130 blockers, vasoactive medications, corticosteroids, immunosuppressive agents, and mechanical ventilation. Exemplary therapies for CRS are described in International Application WO2014011984, which is hereby incorporated by reference.

Tocilizumab is a humanized, immunoglobulin Glkappa anti-human IL-6R monoclonal antibody. See, e.g., id. Tocilizumab blocks binding of IL-6 to soluble and membrane bound IL-6 receptors (IL-6Rs) and thus inhibitos classical and trans-IL-6 signaling. In embodiments, tocilizumab is administered at a dose of about 4-12 mg/kg, e.g., about 4-8 mg/kg for adults and about 8-12 mg/kg for pediatric subjects, e.g., administered over the course of 1 hour.

In some embodiments, the CRS therapeutic is an inhibitor of IL-6 signalling, e.g., an inhibitor of IL-6 or IL-6 receptor. In one embodiment, the inhibitor is an anti-IL-6 antibody, e.g., an anti-IL-6 chimeric monoclonal antibody such as siltuximab. In other embodiments, the inhibitor comprises a soluble gp130 or a fragment thereof that is capable of blocking IL-6 signalling. In some embodiments, the sgp130 or fragment thereof is fused to a heterologous domain, e.g., an Fc domain, e.g., is a gp130-Fc fusion protein such as FE301. In embodiments, the inhibitor of IL-6 signalling comprises an antibody, e.g., an antibody to the IL-6 receptor, such as sarilumab, olokizumab (CDP6038), elsilimomab, sirukumab (CNTO 136), ALD518/BMS-945429, ARGX-109, or FM101. In some embodiments, the inhibitor of IL-6 signalling comprises a small molecule such as CPSI-2364.

Exemplary vasoactive medications include but are not limited to angiotensin-11, endothelin-1, alpha adrenergic agonists, rostanoids, phosphodiesterase inhibitors, endothelin antagonists, inotropes (e.g., adrenaline, dobutamine, isoprenaline, ephedrine), vasopressors (e.g., noradrenaline, vasopressin, metaraminol, vasopressin, methylene blue), inodilators (e.g., milrinone, levosimendan), and dopamine.

Exemplary vasopressors include but are not limited to norepinephrine, dopamine, phenylephrine, epinephrine, and vasopressin. In some embodiments, a high-dose vasopressor includes one or more of the following: norpepinephrine monotherapy at ≥20 ug/min, dopamine monotherapy at ≥10 ug/kg/min, phenylephrine monotherapy at ≥200 ug/min, and/or epinephrine monotherapy at ≥10 ug/min. In some embodiments, if the subject is on vasopres sin, a high-dose vasopressor includes vasopressin+norepinephrine equivalent of ≥10 ug/min, where the norepinephrine equivalent dose=[norepinephrine (ug/min)]+[dopamine (ug/kg/min)/2]+[epinephrine (ug/min)]+[phenylephrine (ug/min)/10]. In some embodiments, if the subject is on combination vasopressors (not vasopressin), a high-dose vasopressor includes norepinephrine equivalent of >20 ug/min, where the norepinephrine equivalent dose=[norepinephrine (ug/min)]+[dopamine (ug/kg/min)/2]+[epinephrine (ug/min)]+[phenylephrine (ug/min)/10]. See e.g., Id.

In some embodiments, a low-dose vasopressor is a vasopressor administered at a dose less than one or more of the doses listed above for high-dose vasopressors.

Exemplary corticosteroids include but are not limited to dexamethasone, hydrocortisone, and methylprednisolone. In embodiments, a dose of dexamethasone of 0.5 mg/kg is used. In embodiments, a maximum dose of dexamethasone of 10 mg/dose is used. In embodiments, a dose of methylprednisolone of 2 mg/kg/day is used.

Exemplary immunosuppressive agents include but are not limited to an inhibitor of TNFα or an inhibitor of IL-1. In embodiments, an inhibitor of TNFα comprises an anti-TNFα antibody, e.g., monoclonal antibody, e.g., infliximab. In embodiments, an inhibitor of TNFα comprises a soluble TNFα receptor (e.g., etanercept). In embodiments, an IL-1 or IL-1R inhibitor comprises anakinra.

In some embodiments, the subject at risk of developing severe CRS is administered an anti-IFN-gamma or anti-sIL2Ra therapy, e.g., an antibody molecule directed against IFN-gamma or sIL2Ra.

In embodiments, for a subject who has received a therapeutic antibody molecule such as blinatumomab and who has CRS or is at risk of developing CRS, the therapeutic antibody molecule is administered at a lower dose and/or a lower frequency, or administration of the therapeutic antibody molecule is halted.

In embodiments, a subject who has CRS or is at risk of developing CRS is treated with a fever reducing medication such as acetaminophen.

In embodiments, a subject herein is administered or provided one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation, in any combination, e.g., in combination with a CAR-expressing cell described herein.

In embodiments, a subject at risk of developing CRS (e.g., severe CRS) (e.g., identified as having a high risk status for developing severe CRS) is administered one or more therapies for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation, in any combination, e.g., in combination with a CAR-expressing cell described herein.

In embodiments, a subject herein (e.g., a subject at risk of developing severe CRS or a subject identified as at risk of developing severe CRS) is transferred to an intensive care unit. In some embodiments, a subject herein (e.g., a subject at risk of developing severe CRS or a subject identified as at risk of developing severe CRS) is monitored for one ore more symptoms or conditions associated with CRS, such as fever, elevated heart rate, coagulopathy, MODS (multiple organ dysfunction syndrome), cardiovascular dysfunction, distributive shock, cardiomyopathy, hepatic dysfunction, renal dysfunction, encephalopathy, clinical seizures, respiratory failure, or tachycardia. In some embodiments, the methods herein comprise administering a therapy for one of the symptoms or conditions associated with CRS. For instance, in embodiments, e.g., if the subject develops coagulopathy, the method comprises administering cryoprecipitate. In some embodiments, e.g., if the subject develops cardiovascular dysfunction, the method comprises administering vasoactive infusion support. In some embodiments, e.g., if the subject develops distributive shock, the method comprises administering alpha-agonist therapy. In some embodiments, e.g., if the subject develops cardiomyopathy, the method comprises administering milrinone therapy. In some embodiments, e.g., if the subject develops respiratory failure, the method comprises performing mechanical ventilation (e.g., invasive mechanical ventilation or noninvasive mechanical ventilation). In some embodiments, e.g., if the subject develops shock, the method comprises administering crystalloid and/or colloid fluids.

In embodiments, the CAR-expressing cell is administered prior to, concurrently with, or subsequent to administration of one or more therapies for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation. In embodiments, the CAR-expressing cell is administered within 2 weeks (e.g., within 2 or 1 week, or within 14 days, e.g., within 14, 13, 12, 11, 10, 9, 8, 7, 6, 5, 4, 3, 2, 1 day or less) of administration of one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation. In embodiments, the CAR-expressing cell is administered at least 1 day (e.g., at least 1, 2, 3, 4, 5, 6, 7, 8, 9, 10, 11, 12, 13, 14, 15, 16, 17, 18, 19, 20, 1, week, 2 weeks, 3 weeks, 4 weeks, 1 month, 2 months, 3 months, 3 months, or more) before or after administration of one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation.

In embodiments, a subject herein (e.g., a subject at risk of developing severe CRS or a subject identified as at risk of developing severe CRS) is administered a single dose of an IL-6 inhibitor or IL-6 receptor (IL-6R) inhibitor (e.g., tocilizumab). In embodiments, the subject is administered a plurality of doses (e.g., 2, 3, 4, 5, 6, or more doses) of an IL-6 inhibitor or IL-6 receptor (IL-6R) inhibitor (e.g., tocilizumab).

In embodiments, a subject at low or no risk of developing CRS (e.g., severe CRS) (e.g., identified as having a low risk status for developing severe CRS) is not administered a therapy for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation.

In some embodiments, the subject treated by the methods disclosed herein has a low severity of CRS, e.g., grade 1, grade 2 or grade 3.

### Pharmaceutical Compositions

Pharmaceutical compositions of the present invention may comprise a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

### Methods of Treating

When “an immunologically effective amount,” “an effective dose”, “an anti-cancer effective amount,” “a cancer-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject).

The dosage of the above treatments to be administered to a subject will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

The administration of the compositions described herein may be carried out in any convenient manner, including by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In one embodiment, the compositions described herein, e.g., comprising a CAR-expressing cell and/or PD-1 inhibitor, are administered to a patient by intradermal or subcutaneous injection. In one embodiment, the the compositions described herein, e.g., comprising a CAR-expressing cell and/or PD-1 inhibitor, are administered by i.v. injection. The the compositions described herein, e.g., comprising a CAR-expressing cell and/or PD-1 inhibitor, may be injected directly into a tumor, lymph node, or site of infection.

It can generally be stated that a pharmaceutical composition comprising the immune effector cells described herein may be administered at a dosage of 10⁴ to 10⁹ cells/kg body weight, in some instances 10⁵ to 10⁶ cells/kg body weight, including all integer values within those ranges. The immune effector cell compositions may also be administered multiple times at these dosages. The cells can be administered by using infusion techniques that are commonly known in immunotherapy (see, e.g., Rosenberg et al., New Eng. J. of Med. 319:1676, 1988).

In certain aspects, it may be desired to administer activated immune effector cells to a subject and then subsequently redraw blood (or have an apheresis performed), activate the cells therefrom according to the present invention, and reinfuse the patient with these activated and expanded cells. This process can be carried out multiple times every few weeks. In certain aspects, the cells can be activated from blood draws of from 10 cc to 400 cc. In certain aspects, the cells are activated from blood draws of 20 cc, 30 cc, 40 cc, 50 cc, 60 cc, 70 cc, 80 cc, 90 cc, or 100 cc.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more CAR constructs of the invention may be introduced, thereby creating a CAR T cell of the invention. Subjects in need thereof may subsequently undergo standard treatment with high dose chemotherapy followed by peripheral blood stem cell transplantation. In certain aspects, following or concurrent with the transplant, subjects receive an infusion of the expanded CAR expressing cells of the present invention. In an additional aspect, expanded cells are administered before or following surgery.

In one embodiment, the CAR is introduced into immune effector cells, e.g., using in vitro transcription, and the subject (e.g., human) receives an initial administration of CAR-expressing cells of the invention, and one or more subsequent administrations of the CAR-expressing cells of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR-expressing cells of the invention are administered to the subject (e.g., human) per week, e.g., 2, 3, or 4 administrations of the CAR-expressing cells of the invention are administered per week. In one embodiment, the subject (e.g., human subject) receives more than one administration of the CAR-expressing cells per week (e.g., 2, 3 or 4 administrations per week) (also referred to herein as a cycle), followed by a week of no CAR-expressing cells administration, and then one or more additional administration of the CAR-expressing cells (e.g., more than one administration of the CAR-expressing cells per week) is administered to the subject. In another embodiment, the subject (e.g., human subject) receives more than one cycle of CAR-expressing cells, and the time between each cycle is less than 10, 9, 8, 7, 6, 5, 4, or 3 days. In one embodiment, the CAR-expressing cells are administered every other day for 3 administrations per week. In one embodiment, the CAR-expressing cells of the invention are administered for at least two, three, four, five, six, seven, eight or more weeks.

In some embodiments, a dose of CAR-expressing cells (e.g., CAR-expressing cells described herein, e.g., CD19 CAR-expressing cells described herein) comprises about 10⁴ to about 10⁹ cells/kg, e.g., about 10⁴ to about 10⁵ cells/kg, about 10⁵ to about 10⁶ cells/kg, about 10⁶ to about 10⁷ cells/kg, about 10⁷ to about 10⁸ cells/kg, or about 10⁸ to about 10⁹ cells/kg. In embodiments, the dose of CAR-expressing cells comprises about 0.6×10⁶ cells/kg to about 2×10⁷ cells/kg. In some embodiments, a dose of CAR-expressing cells described herein (e.g., CD19 CAR-expressing cell) comprises about 2×10⁵, 1×10⁶, 1.1×10⁶, 2×10⁶, 3×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg. In some embodiments, a dose of CAR cells (e.g., CD19 CAR-expressing cell) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg. In some embodiments, a dose of CAR cells (e.g., CD19 CAR-expressing cell) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, 3×10⁸, or 5×10⁸ cells/kg. In some embodiments, a dose of CAR cells (e.g., CD19 CAR-expressing cell) comprises about 1.1×10⁶-1.8×10⁷ cells/kg. In some embodiments, a dose of CAR cells (e.g., CD19 CAR-expressing cell) comprises about 1×10⁷, 2×10⁷, 5×10⁷, 1×10⁸, 2×10⁸, 3×10⁸, 5×10⁸, 1×10⁹, 2×10⁹, or 5×10⁹ cells. In some embodiments, a dose of CAR cells (e.g., e.g., CD19 CAR-expressing cell) comprises at least about 1×10⁷, 2×10⁷, 5×10⁷, 1×10⁸, 2×10⁸, 3×10⁸, 5×10⁸, 1×10⁹, 2×10⁹, or 5×10⁹ cells. In some embodiments, a dose of CAR cells (e.g., e.g., CD19 CAR-expressing cell) comprises up to about 1×10⁷, 2×10⁷, 5×10⁷, 1×10⁸, 2×10⁸, 3×10⁸, 5×10⁸, 1×10⁹, 2×10⁹, or 5×10⁹ cells.

In some embodiments, a dose of CAR cells (e.g., CD19 CAR-expressing cell) comprises up to about 1×10⁷, 1.5×10⁷, 2×10⁷, 2.5×10⁷, 3×10⁷, 3.5×10⁷, 4×10⁷, 5×10⁷, 1×10⁸, 1.5×10⁸, 2×10⁸, 2.5×10⁸, 3×10⁸, 3.5×10⁸, 4×10⁸, 5×10⁸, 1×10⁹, 2×10⁹, or 5×10⁹ cells. In some embodiments, a dose of CAR cells (e.g., CD19 CAR-expressing cell) comprises up to about 1-3×10⁷ to 1-3 ×10⁸. In some embodiments, the subject is administered about 1-3×10⁷ of CD19 CAR-expressing cells. In other embodiments, the subject is administered about 1-3×10⁸ of CD19 CAR-expressing cells.

In some embodiments, a dose of CAR-expressing cells (e.g., CAR-expressing cells described herein, e.g., CD19 CAR-expressing cells described herein) comprises about 1×10⁶ cells/m² to about 1×10⁹ cells/m², e.g., about 1×10⁷ cells/m² to about 5×10⁸ cells/m², e.g., about 1.5×10⁷ cells/m², about 2×10⁷ cells/m², about 4.5×10⁷ cells/m², about 10⁸ cells/m², about 1.2×10⁸ cells/m², or about 2×10⁸ cells/m².

In embodiments, the CD19 CAR-expressing cells are administered in a plurality of doses, e.g., a first dose, a second dose, and optionally a third dose. In embodiments, the method comprises treating a subject (e.g., an adult subject) having a cancer (e.g., acute lymphoid leukemia (ALL)), comprising administering to the subject a first dose, a second dose, and optionally one or more additional doses, each dose comprising immune effector cells expressing a CAR molecule, e.g., a CD19 CAR molecule, e.g., a CAR molecule according to SEQ ID NO: 108.

In embodiments, the method comprises administering a dose of 2-5×10⁶ viable CAR-expressing cells/kg, wherein the subject has a body mass of less than 50 kg; or administering a dose of 1.0 -2.5 ×10⁸ viable CAR-expressing cells, wherein the subject has a body mass of at least 50 kg.

In embodiments, a single dose is administered to the subject, e.g., pediatric subject.

In embodiments, the doses are administered on sequential days, e.g., the first dose is administered on day 1, the second dose is administered on day 2, and the optional third dose (if administered) is administered on day 3.

In embodiments, a fourth, fifth, or sixth dose, or more doses, are administered.

In embodiments, the first dose comprises about 10% of the total dose, the second dose comprises about 30% of the total dose, and the third dose comprises about 60% of the total dose, wherein the aforementioned percentages have a sum of 100%. In embodiments, the first dose comprises about 9-11%, 8-12%, 7-13%, or 5-15% of the total dose. In embodiments, the second dose comprises about 29-31%, 28-32%, 27-33%, 26-34%, 25-35%, 24-36%, 23-37%, 22-38%, 21-39%, or 20-40% of the total dose. In embodiments, the third dose comprises about 55-65%, 50-70%, 45-75%, or 40-80% of the total dose. In embodiments, the total dose refers to the total number of viable CAR-expressing cells administered over the course of 1 week, 2 weeks, 3 weeks, or 4 weeks. In some embodiments wherein two doses are administered, the total dose refers to the sum of the number of viable CAR-expressing cells administered to the subject in the first and second doses. In some embodiments wherein three doses are administered, the total dose refers to the sum of the number of viable CAR-expressing cells administered to the subject in the first, second, and third doses.

In embodiments, the dose is measured according to the number of viable CAR-expressing cells therein. CAR expression can be measured, e.g., by flow cytometry using an antibody molecule that binds the CAR molecule and a detectable label. Viability can be measured, e.g., by Cellometer.

In embodiments, the viable CAR-expressing cells are administered in ascending doses. In embodiments, the second dose is larger than the first dose, e.g., larger by 10%, 20%, 30%, or 50%. In embodiments, the second dose is twice, three times, four times, or five times the size of the first dose. In embodiments, the third dose is larger than the second dose, e.g., larger by 10%, 20%, 30%, or 50%. In embodiments, the third dose is twice, three times, four times, or five times the size of the second dose.

In certain embodiments, the method includes one, two, three, four, five, six, seven or all of a)-h) of the following:

a) the number of CAR-expressing, viable cells administered in the first dose is no more than 1/3, of the number of CAR-expressing, viable cells administered in the second dose;

b) the number of CAR-expressing, viable cells administered in the first dose is no more than 1/X, wherein X is 2, 3, 4, 5, 6, 7, 8, 9, 10, 15, 20, 30, 40 or 50, of the total number of CAR-expressing, viable cells administered;

c) the number of CAR-expressing, viable cells administered in the first dose is no more than 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, or 5×10⁸ CAR-expressing, viable cells, and the second dose is greater than the first dose;

d) the number of CAR-expressing, viable cells administered in the second dose is no more than 1/2, of the number of CAR-expressing, viable cells administered in the third dose;

e) the number of CAR-expressing, viable cells administered in the second dose is no more than 1/Y, wherein Y is 2, 3, 4, 5, 6, 7, 8, 9, 10, 15, 20, 30, 40 or 50, of the total number of CAR-expressing, viable cells administered;

f) the number of CAR-expressing, viable cells administered in the second dose is no more than 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, or 5×10⁸ CAR-expressing, viable cells, and the third dose is greater than the second dose;

h) the dosages and time periods of administration of the first, second, and optionally third doses are selected such that the subject experiences CRS at a level no greater than 4, 3, 2, or 1.

In embodiments, the total dose is about 5×10⁸ CAR-expressing, viable cells. In embodiments, the total dose is about 5×10⁷-5×10⁸ CAR-expressing, viable cells. In embodiments, the first dose is about 5×10⁷ (e.g., ±10%, 20%, or 30%) CAR-expressing, viable cells, the second dose is about 1.5×10⁸ (e.g., ±10%, 20%, or 30%) CAR-expressing, viable cells, and the third dose is about 3×10⁸ (e.g., ±10%, 20%, or 30%) CAR-expressing, viable cells.

In embodiments, the subject is evaluated for CRS after receiving a dose, e.g., after receiving the first dose, the second dose, and/or the third dose.

In embodiments, the subject receives a CRS treatment, e.g., tocilizumab, a corticosteroid, etanercept, or siltuximab. In embodiments, the CRS treatment is administered before or after the first dose of cells comprising the CAR molecule. In embodiments, the CRS treatment is administered before or after the second dose of cells comprising the CAR molecule. In embodiments, the CRS treatment is administered before or after the third dose of cells comprising the CAR molecule. In embodiments, the CRS treatment is administered between the first and second doses of cells comprising the CAR molecule, and/or between the second and third doses of cells comprising the CAR molecule.

In embodiments, in a subject having CRS after the first dose, e.g., CRS grade 1, 2, 3, or 4, the second dose is administered at least 2, 3, 4, or 5 days after the first dose. In embodiments, in a subject having CRS after the second dose, e.g., CRS grade 1, 2, 3, or 4, the third dose is administered at least 2, 3, 4, or 5 days after the second dose. In embodiments, in a subject having CRS after the first dose, the second dose of CAR-expressing cells is delayed relative to when the second dose would have been administered had the subject not had CRS. In embodiments, in a subject having CRS after the second dose, the third dose of CAR-expressing cells is delayed relative to when the third dose would have been administered had the subject not had CRS.

In embodiments, the subject has a cancer with a high disease burden before the first dose is administered. In embodiments, the subject has bone marrow blast levels of at least 1%, 2%, 3%, 4%, 5%, 6%, 7%, 8%, 9%, 10%, 15%, 20%, 25%, 30%, 35%, 40%, 45%, or 50%, e.g., at least 5%. In embodiments, the subject has a cancer in stage I, II, III, or IV. In embodiments, the subject has a tumor mass of at least 1, 2, 5, 10, 20, 50, 100, 200, 500, or 1000 g, e.g., in a single tumor or a plurality of tumors.

In some embodiments, the subject has cancer (e.g., a solid cancer or a hematological cancer as described herein). In an embodiment, the subject has CLL. In embodiments, the subject has ALL. In other embodiments, the subject has multiple myeloma.

In one embodiment, the cancer is a disease associated with CD19 expression, e.g., as described herein. In other embodiments, the cancer is a disease associated with a tumor antigen, e.g., as described herein. In embodiments, the CAR molecule is a CAR molecule as described herein.

In one aspect, CAR-expressing cells, e.g., CD19 CAR-expressing cells, are generated using lentiviral viral vectors, such as lentivirus. CAR-expressing cells generated that way will have stable CAR expression.

In one aspect, the CAR-expressing cells transiently express CAR vectors for 4, 5, 6, 7, 8, 9, 10, 11, 12, 13, 14, 15 days after transduction. Transient expression of CARs can be effected by RNA CAR vector delivery. In one aspect, the CAR RNA is transduced into the T cell by electroporation.

A potential issue that can arise in patients being treated using transiently expressing CAR-expressing cells (particularly with murine scFv bearing CAR-expressing cells) is anaphylaxis after multiple treatments.

Without being bound by this theory, it is believed that such an anaphylactic response might be caused by a patient developing humoral anti-CAR response, i.e., anti-CAR antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-CAR antibody response during the course of transient CAR therapy (such as those generated by RNA transductions), CAR-expressing cell infusion breaks should not last more than ten to fourteen days.

Using CARs with human (instead of murine) scFvs can reduce the likelihood and intensity of a patient having an anti-CAR response.

Dosages and therapeutic regimens of the PD-1 inhibitor, e.g., anti-PD-1 antibody molecule, can be determined by a skilled artisan. Suitable dosages of the molecules used will depend on the age and weight of the subject and the particular drug used.

Methods of administering the antibody molecules are known in the art and are described below. Suitable dosages of the molecules used will depend on the age and weight of the subject and the particular drug used. Dosages and therapeutic regimens of the anti-PD-1 antibody molecule can be determined by a skilled artisan.

In certain embodiments, the anti-PD-1 antibody molecule is administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg. In some embodiments, the anti-PD-1 antibody molecule is administered at a dose of about 1 mg/kg, about 3 mg/kg, about 5 mg/kg, about 10 mg/kg, about 20 mg/kg, about 30 mg/kg, or about 40 mg/kg. In some embodiments, the anti-PD-1 antibody molecule is administered at a dose of about 1-3 mg/kg, or about 3-10 mg/kg. In some embodiments, the anti-PD-1 antibody molecule is administered at a dose of about 0.5-2, 2-4, 2-5, 5-15, or 5-20 mg/kg. The dosing schedule can vary from e.g., once a week to once every 2, 3, or 4 weeks. In one embodiment, the anti-PD-1 antibody molecule is administered at a dose from about 10 to 20 mg/kg every other week. In another embodiment, the anti-PD-1 antibody molecule is administered at a dose of about 1 mg/kg once every two weeks, about 3 mg/kg once every two weeks, 10 mg/kg once every two weeks, 3 mg/kg once every four weeks, or 5 mg/kg once every four weeks.

In other embodiments, the anti-PD-1 antibody molecule is administered by injection (e.g., subcutaneously or intravenously) at a dose (e.g., a flat dose) of about 200 mg to 500 mg, e.g., about 250 mg to 450 mg, about 300 mg to 400 mg, about 250 mg to 350 mg, about 350 mg to 450 mg, or about 300 mg or about 400 mg. In some embodiments, the anti-PD-1 antibody molecule is administered at a dose of about 200 mg, about 250 mg, about 300 mg, about 350 mg, about 400 mg, about 450 mg, or about 500 mg. In some embodiments, the anti-PD1 antibody is administered at a dose of 200 or 300 mg. In some embodiments, the anti-PD-1 antibody molecule is administered at a dose of about 250-450 mg, or about 300-400 mg. In some embodiments, the anti-PD-1 antibody molecule is administered at a dose of about 200-300 mg, 250-350 mg, 300-400 mg, 350-450 mg, or 400-500 mg. The dosing schedule can vary from e.g., once a week to once every 2, 3, 4, 5, or 6 weeks. In one embodiment the anti-PD-1 antibody molecule is administered at a dose from about 300 mg to 400 mg once every three or once every four weeks. In one embodiment, the anti-PD-1 antibody molecule is administered at a dose from about 300 mg once every three weeks. In one embodiment, the anti-PD-1 antibody molecule is administered at a dose from about 400 mg once every four weeks. In one embodiment, the anti-PD-1 antibody molecule is administered at a dose from about 300 mg once every four weeks. In one embodiment, the anti-PD-1 antibody molecule is administered at a dose from about 400 mg once every three weeks. The anti-PD-1 antibody can be administered one or more times, e.g., one, two, three, four, five, six, seven or more times. In one embodiment, the anti-PD-1 antibody is administered six times. The anti-PD-1 antibody can be administered at least 5 days, e.g., about 5, 6, 7, 8, 9, 10, 11, 12, 13, 14, 20, 25, 30, 35, or 40 days, after administration of CAR-expressing cells, e.g., CD19 (e.g., CLT019 or CTL119) or BCMA CAR expressing cells. In some embodiments, the anti-PD-1 antibody can be administered about 8 days or about 15 days after administration of CAR-expressing cells, e.g., CD19 expressing cells (e.g., CLT019 or CTL119) or BCMA CAR expressing cells.

The antibody molecules can be administered by a variety of methods known in the art, although for many therapeutic applications, the preferred route/mode of administration is intravenous injection or infusion. For example, the antibody molecules can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the antibody molecules can be administered by intravenous infusion at a rate of less than 10 mg/min; preferably less than or equal to 5 mg/min to reach a dose of about 1 to 100 mg/m ²,preferably about 5 to 50 mg/m², about 7 to 25 mg/m² and more preferably, about 10 mg/m². As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The antibody molecule can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, the antibody molecule can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the antibody is infused over a period of about 30 min.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### CD19 CAR-Expressing Cell and PD-1 Inhibitor Decreased Tumor Burden in a Human Subject

A 34 year old woman with follicular lymphoma transformed to “double hit” DLBCL was treated with a CD19 CART cell infusion in combination with a PD-1 antagonist. The woman had previously undergone eleven lines of chemotherapy and immunotherapy, including allogeneic bone marrow transplant, but was non-responsive to the previous therapy. The woman underwent lymphodepleting chemotherapy (e.g., carboplatin and gemcitabine) prior to administration with a CD19 CART cell (CTL019). CTL019 was administered to the woman, followed by radiation therapy, and then a PD-1 antagonist, pembrolizumab (a humanized IgG4 anti-PD-1 monoclonal antibody). A biopsy was taken between administration of the CTL019 and the radiation therapy—the biopsy was analyzed by flow cytometry, immunohistochemistry (IHC), and fluorescence in situ hybridization (FISH). By flow cytometry, the sample was positive for a kappa light chain, CD10, and CD19. By IHC, the sample had large PAXS+ B cells and was PDL1+. By FISH, the sample had rearranged c-MYC and BCL-2. A second biopsy was taken after pembrolizumab treatment. In the second biopsy, extensive necrosis was observed, and no tumor was detected. Thus, the data demonstrate that the combination of a CD19 CART cell with a PD-1 antagonist was effective in reducing tumor burden in a human.

### Example 2

### Characterization of a PD-1 Antagonist, PDR-001

PDR-001 is a humanized monoclonal antibody directed against human PD-1. PDR-001 has a stabilizing hinge mutation to prevent molecule dissociation and formation of half-antibodies. PDR-001 belongs to the IgG4/kappa isotype subclass.

PDR-001 was characterized in vitro for its affinity and activity on human PD-1. PDR-001 was expressed in a CHO cell line. PDR-001 bound with high affinity to human PD-1. In Biacore assays, the Kd of PDR-001 on human PD-1 was 0.83 nM. In lymphocyte stimulation assays using human blood ex vivo, PDR-001 enhanced interleukin-2 (IL-2) production by approximately 2 fold in response to super antigen stimulation with Staphylococcus enterotoxin B (SEB). PDR-001 did not cross react with rodent PD-1 but did cross react with cynomolgus monkey PD-1 and was functionally active, thereby making cynomolgus monkey a relevant species for toxicology studies. The affinity of PDR-001 for cynomolgus PD-1 was 0.93 nM, which is similar to the Kd for human PD-1.

In addition, non-clinical toxicology of PDR-001 was evaluated in a five-week good laboratory practice (GLP) toxicology study in cynomolgus monkeys with safety pharmacology endpoints and an eight week recovery. Doses as high as 100 mg/kg/week were evaluated without drug-related in-life, mortality, organ weight changes, or macroscopic findings. At the highest doses tested, macrophage infiltrates in the spleen and limited mononuclear infiltrates in the vascular and perivascular space were noted.

### Example 3

### Clinical Results with PDR-001

A clinical study was performed on PDR-001 in patients with advanced malignancies . Patients were treated at dose levels of 1, 3, and 10 mg/kg Q2W and 3 and 5 mg/kg Q4W. None of the patients experienced a dose limiting toxicitiy, and the toxicity profile appeared similar to that of marketed inhibitors of PD-1. The pharmacokinetic data obtained from the dose escalation and modeling of the exposure data supported the use of flat dosing for PDR-001 of 400 mg administered every 4 weeks. The trough concentrations (Ctrough) were in line with observed steady state mean trough concentrations for pembrolizumab, which is approved with substantial efficacy in several cancer types. The data also supported the use of 300 mg Q3W as an alternative dose regimen, e.g., in combination treatment regimens.

### Example 4

### Clinical Study Using Combination of CTL019 and PDR-001

Subjects in the study have diffuse large B cell lymphoma (DLBCL) that has been identified as CD19+. Subjects have one or more of the following characteristics: (i) residual disease after primary therapy and as such no eligible for autologous stem cell transplant; (ii) relapsed or persistent disease after prior autologous stem cell transplant; (iii) beyond first complete response (CR) with relapsed or persistent disease and not eligible or appropriate for conventional allogeneic or autologous stem cell transplant; and/or (iv) antecedent history of follicular lymphoma or CLL/SLL.

Subjects receive an infusion of CART-19 (e.g., CTL019 cells, e.g., described in detail herein). The CART-19 cells are cryopreserved in infusible cryomedia and are administered as a single infusion. Each bag of cells contains cryomedia containing the following infusible grade reagents (% v/v): 31.25% plasmalyte-A, 31.25% dextrose (5%), 0.45% NaCl, up to 7.5% DMSO, 1% dextran 40, and 5% human serum albumin. A single dose of CART-19 cells is administered intravenously by an infusion containing 1-5×10⁸ cells transduced with the CD19 TCζ/4-1BB vector. The infusion occurs approximately 1-4 days following chemotherapy. The CART-19 is a murine CART-19 (e.g., CTL019).

Subjects also receive PDR-001. PDR-001 is expressed in CHO cells. The PDR-001 formulation is a lyophilized powder in a vial, 100 mg/lyophilisate per vial. After reconstitution of the lyophilized powder with 1.0 mL water for injection, the resulting solution contains 100 mg/mL PDR-001, histidine/histidine-HCl, sucrose, polysorbate-20 at pH 5.5. If no cytokine release syndrome (CRS) has developed, then PDR-001 is administered after CART-19 infusion. If CRS has developed after CART-19 infusion, then PDR-001 is administered after CRS has resolved.

### Example 5

### Low Dose RAD001 Stimulates CART Proliferation in a Cell Culture Model

The effect of low doses of RAD001 on CAR T cell proliferation in vitro is described, e.g., in Example 8 of US2016/0096892A1, and the entirety of the application is herein incorporated by reference.

### Example 6

### Low Dose RAD001 Stimulates CART Expansion In Vivo

The effect of low dose RAD001 on CART expansion in vivo is described, e.g., in Example 9 of US2016/0096892A1, and the entirety of the application is herein incorporated by reference.

### Example 7

### PD-1 Blockade Modulated Chimeric Antigen Receptor (CAR) Modified T Cells and Induced Tumor Regression

Antibodies blocking the programmed death 1 receptor (PD-1) on T cells produce tumor regression in multiple cancers by disrupting the PD-L1/PD-1 immune inhibitory axis. See, e.g., Topalian et al. N. Engl J Med 2012:366:2443-54: Brahmer et al. N Engl J Med 2012; 366:2455-65; Hamid, et al, N Engl J Med 2013; 369:134-44; Wolchok, et al. N Engl J Med 2013; 369:122-33; and Topalian, et at. J Clin Oncol 2014; 32:1020-30. This approach to cancer immunotherapy may be a good partner for chimeric antigen receptor (CAR) modified T cell therapies but the combination has not yet been tested. This example describes experiments in which a PD-1 blocking antibody was administered to a patient with refractory diffuse large B cell lymphoma (DLBCL) and progressive lymphoma after therapy with CAR modified T cells directed against CD19 (CART19). Following PD-1 blockade, the patient had a robust antitumor response, an expansion of CART19 cells, and decreased co-expression of PD-1 and Eomes by CART19 cells. These results suggest that anti-PD-1 can be highly effective against cancers failing to respond to CAR modified T cell therapy. It also suggests that the PD-1 pathway may be important in determining the response to CAR modified T cell immunotherapy.

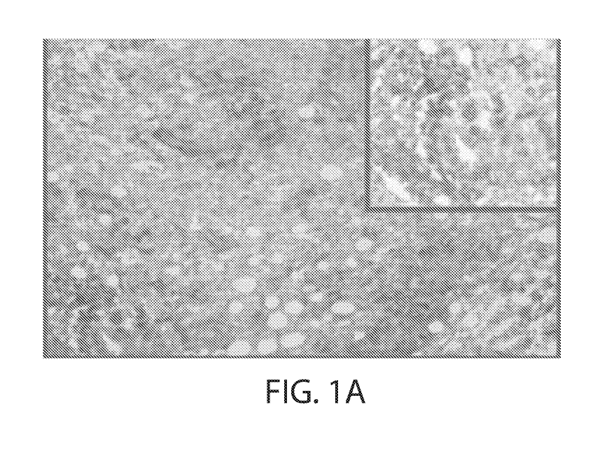

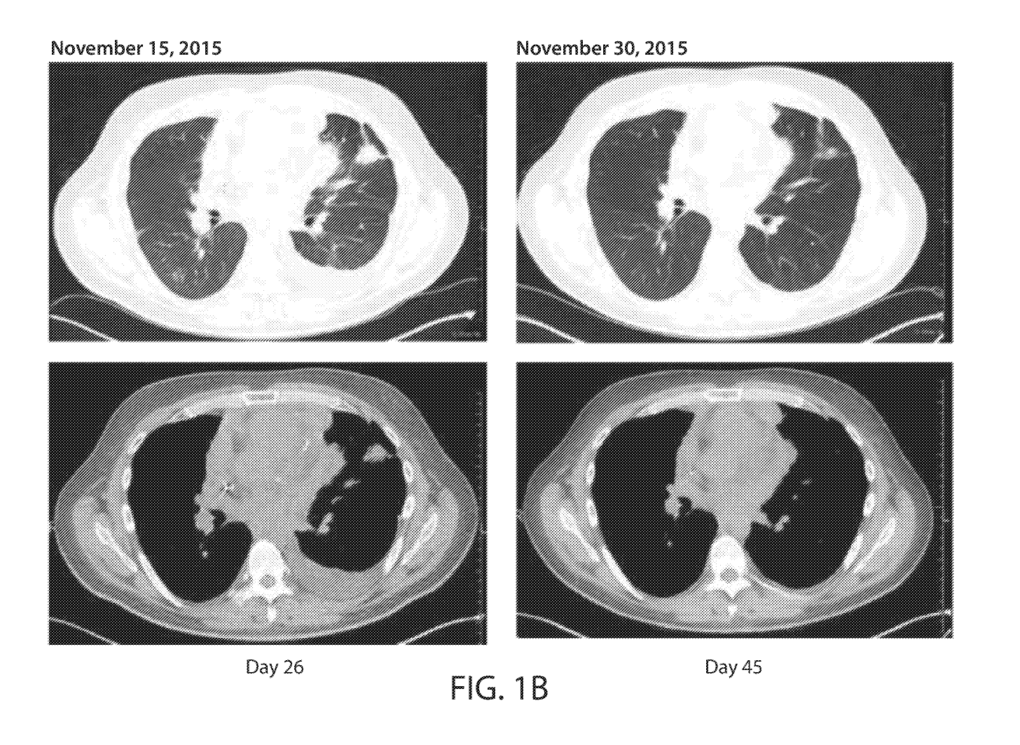

A 35-year-old man with multiply pretreated, refractory DLBCL of primary mediastinal origin with extranodal involvement of small intestine at diagnosis, and mediastinum, lung, myocardium, and pericardium at progression, was treated on a clinical trial at the University of Pennsylvania with autologous CART19 cells expressing murine anti-CD19 scFv and 4-1BB—CD3ζ costimulatory—activation domains (NCT02030834). See Schuster et al. Blood 2015; 126(23):183(abstract). CART19 cells were manufactured as previously described. See, e.g., Porter Sci Transl Med 2015; 7(303):303ra139; and Milone et al. Mol Ther 2009; 17(8):1453-64. The patient received lymphodepleting chemotherapy with hyperfractionated cyclophosphamide (300 mg/m²×6 doses), followed by autologous CART19 cell infusion (5×10⁸ CART19 cells or 5.34×10⁶ cells/kg) on October 16, 2015. Follow-up chest CT scan performed on Nov. 11, 2015, to evaluate worsening dyspnea showed progressive lymphoma with enlargement of mediastinal and pericardial tumor as well as new and enlarging pulmonary nodules (FIG. 1A). Cardiac MRI documented myocardial and pericardial invasion. In view of the patient's clinical status with rapidly progressive hypoxia and respiratory distress, mediastinoscopy or thorascopic lung biopsy was not performed. Thus, it was not possible to exclude pseudoprogression as the cause of mediastinal lymph node and pulmonary parenchymal lesions enlargement following CTL019. He received pembrolizumab, 2 mg/kg, on Nov. 11, 2015. Pembrolizumab was chosen for therapy because of preclinical data indicating that anti-PD-1 therapy potently enhances the eradication of established tumors by gene-modified T cells (see, e.g., John et al. Clin Cancer Res 2013; 19(20):5636-46) and the patient's tumor cells strongly expressed PD-L1 (FIG. 1B). Other than fever, therapy was well tolerated. By Nov. 30, 2015, significant clinical improvement was noted; chest CT at that time showed interval improvement of multiple pulmonary nodules, pleural effusion, mediastinal lymphadenopathy, and pericardial nodularity (FIG. 1A). Thus, pseudoprogression after CART19 was considered unlikely since there was reduction in the size of lesiosn after administration of pembrollizumab, rather than further progression. By 3 weeks after therapy, he was able to return to work. Pembrolizumab, 2 mg/kg, was continued every 3 weeks; PET/CT scans on Dec. 22, 2015 and Apr. 20, 2016 showed continued anatomic improvement in mediastinal adenopathy with residual FDG uptake (partial metabolic response); pulmonary involvement by lymphoma had resolved. Twelve months after initiation of pembrolizumab, the patient continues to be clinically well.

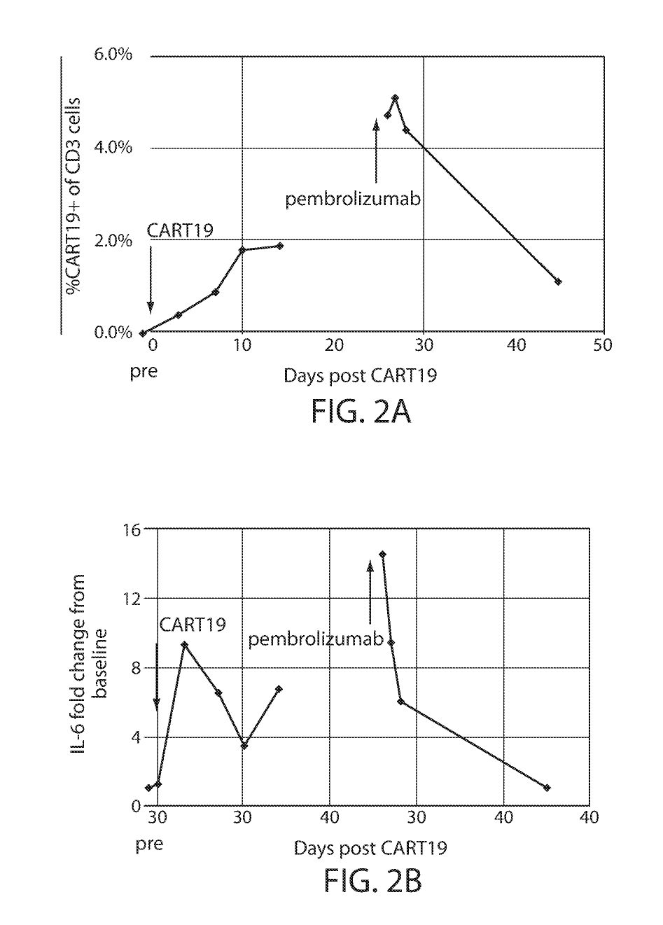

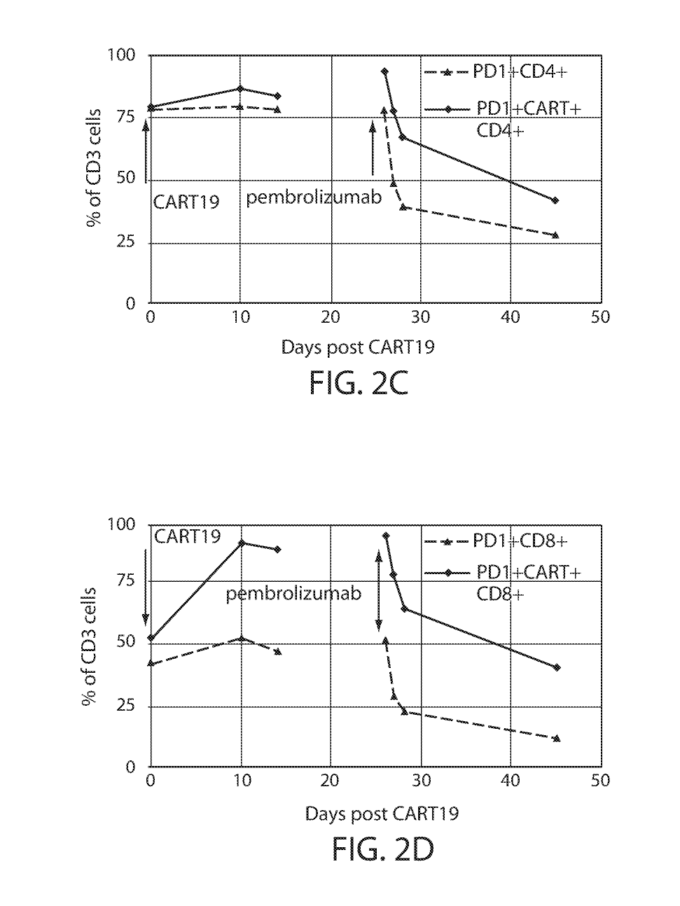

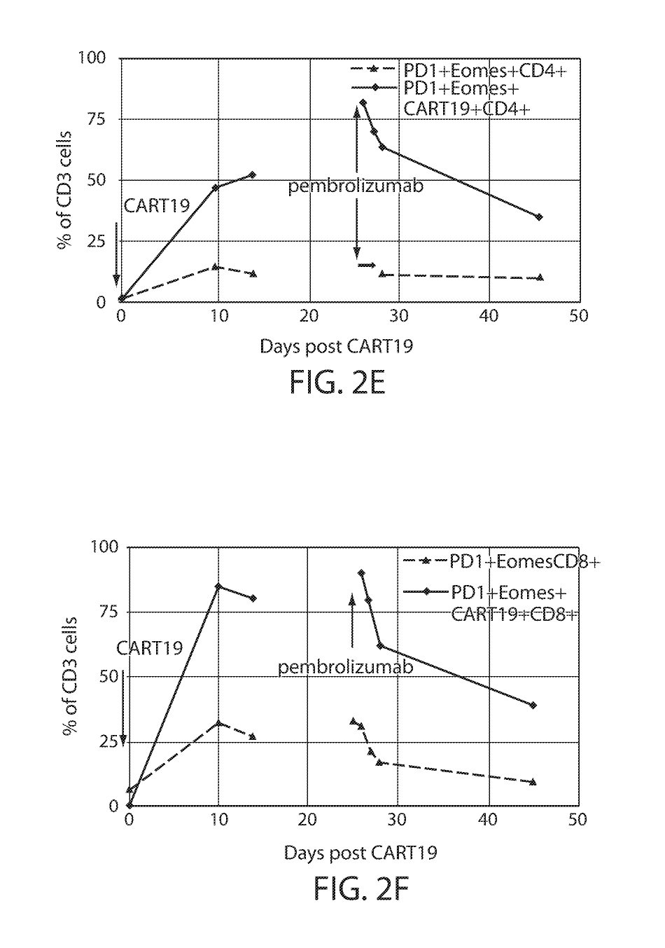

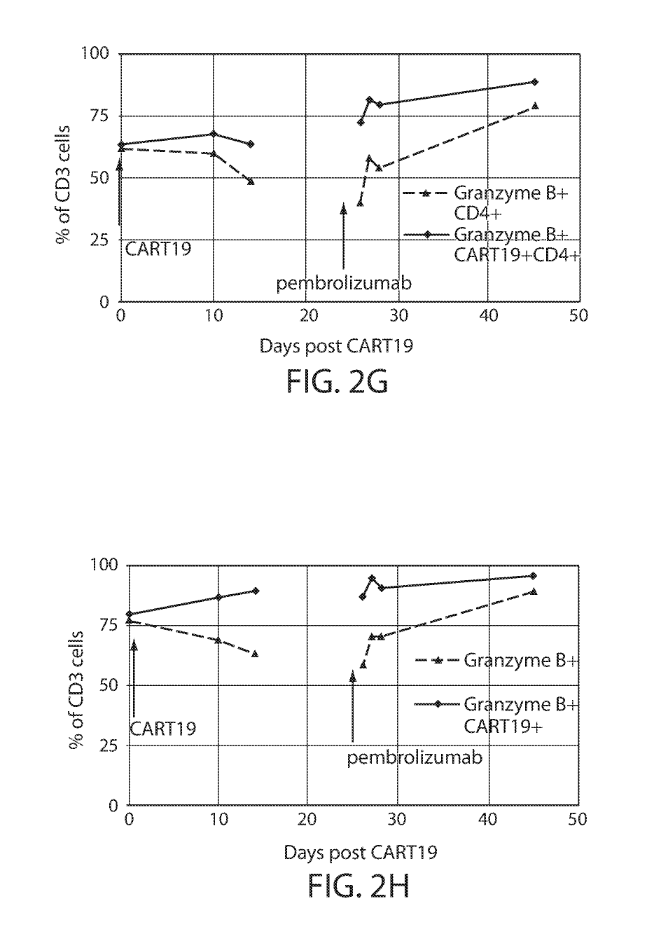

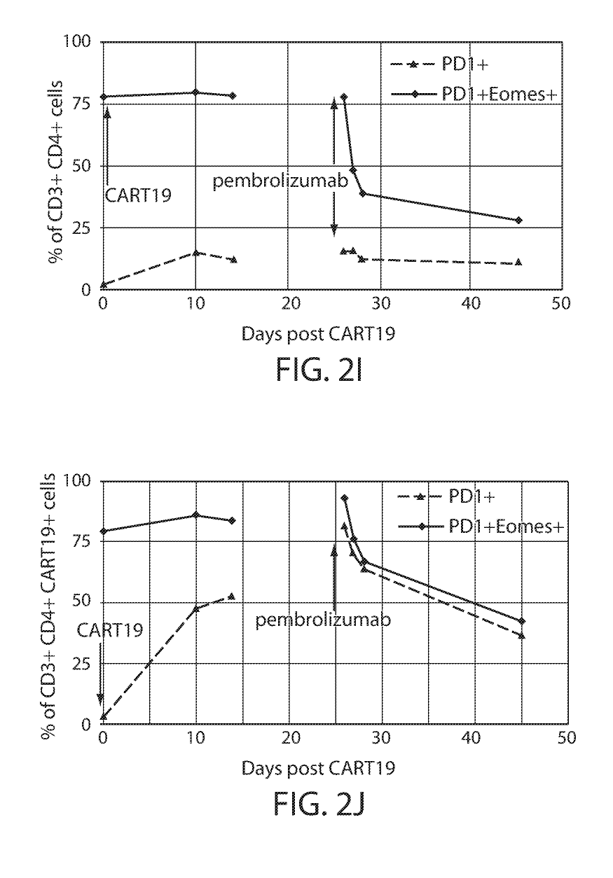

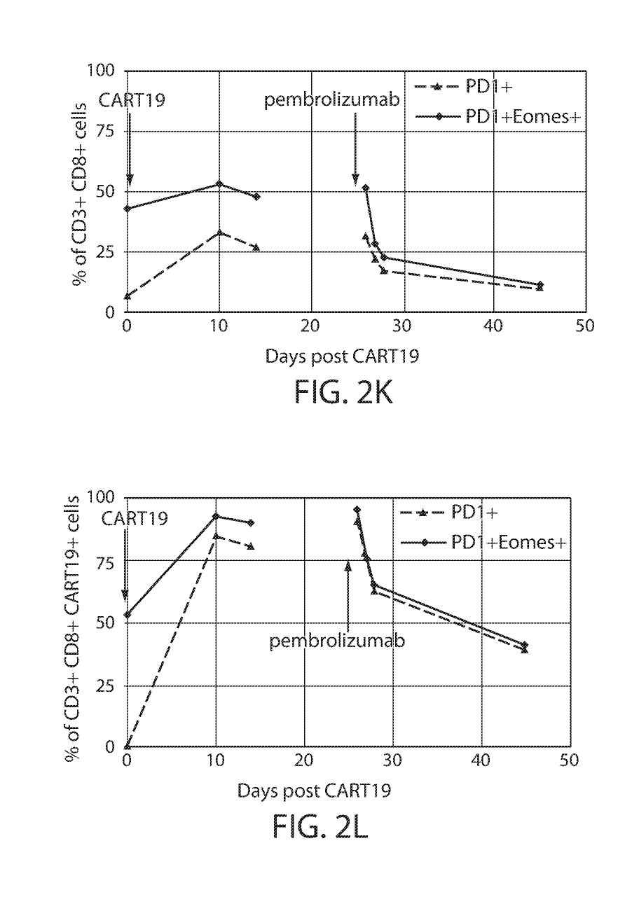

Peripheral blood was analyzed for changes in CART19 DNA by qPCR (data not shown), percentage CART19 cells by flow cytometry, and changes in serum cytokines (FIGS. 2A,-2B). See Porter et al. Sci Transl Med 2015; 7(303):303ra139. CART19 DNA copy number increased to a maximum of 2,350 copies per mcg DNA following CART19 cell infusion and increased again from 497 copies per mcg on day 14 before pembrolizumab to 1,530 copies per mcg on day 26 after pembrolizumab with an apparent sustained increase after starting pembrolizumab. The percentage CAR19-expressing T cells increased after CART19 infusion stabilizing around days 10-14; however, for 48 hours after pembrolizumab, the highest percentages CAR19+ T cells were observed (FIG. 2A). This reflects an increase in both CAR19+ CD8+ and CD4+ T cells after pembrolizumab, particularly the CART19+CD8+ cells (data not shown). The highest serum IL-6 levels were observed days 3-7 following CART19 infusion and during the 24 hours after pembrolizumab (FIG. 2B). After pembrolizumab infusion, CART19 cells co-expressing PD1/Eomes decreased, in the CD4+ CART19+ cells (FIGS. 2E, 2I and 2J) and CD8+ CART19+ cells (FIGS. 2F, 2K and 2L). No changes were observed in cells co-expressing PD-1 and CTLA4, TIM3, or LAG3 (data not shown). Granzyme B+ expression increased after pembrolizumab in both T cell subsets, particularly in CART19+ CD8+ cells (FIGS. 2G-2H).

TCRβ deep sequencing was performed on the apheresis product, the CART19 transduced cell product, and on peripheral blood at day 14 (prior to pembrolizumab), at day 26 (1 hour after pembrolizumab), and at day 45 (19 days after pembrolizumab). Increases in richness (productive rearrangements) and in productive clonality after pembrolizumab were observed (data not shown). Eight dominant clones (frequency ≥1%, range 1.2%-13.1%) were observed after pembrolizumab. Two of these clones initially expanded after CART19 infusion (day 14, clone 1: 6.1%, clone 2: 2.4%) and continued to further expand after pembrolizumab (days 26 and 45, clone 1: 6.1% to 13.11% and clone 2: 2.9% to 6.45%). Four clones were present at low levels after CART19 and expanded after pembrolizumab (days 14 to 26 to 45, clone 4: 0.4% to 0.4% to 2.1%, clone 5: 0.1% to 0.3% to 1.5%, clone 7: 0.6% to 0.9% to 1.3%, clone 8: 0.1% to 0.3% to 1.2%); and two dominant clones were only present after pembrolizumab (days 14 to 26 to 45, clone 3: 0% to 0.27% to 3.57%, clone 6: 0% to 0.04% to 1.46%). The clinical observations combined with the correlative laboratory findings suggest that pembrolizumab may enhance the efficacy CART19 cells in addition to possibly inducing proliferation of other tumor-directed clones. This also suggests a potentially important role for the PD-1/PD-L1 pathway in CAR modified T cell immunotherapy in general. Based on the results described herein, a phase I/II clinical trial of pembrolizumab in patients with CD19+ lymphomas failing to respond to CART19 therapy (NCT02650999) is being performed.

### Example 8

### Low Levels of Immune Checkpoint Molecules are Associated with Improved Outcomes

Immune checkpoint molecules (PD-L1, PD1, LAG3, and TIM3) were detected in samples from lymphoma patients by immunohistochemistry. Positive and negative control tissues and cell lines were also performed. The immune checkpoint expression analysis was performed using quantitative image analysis on a region of interest which can include tumor cells and non-tumor cells such as immune cells. Samples were taken from tissue, lymph node, or bone marrow.

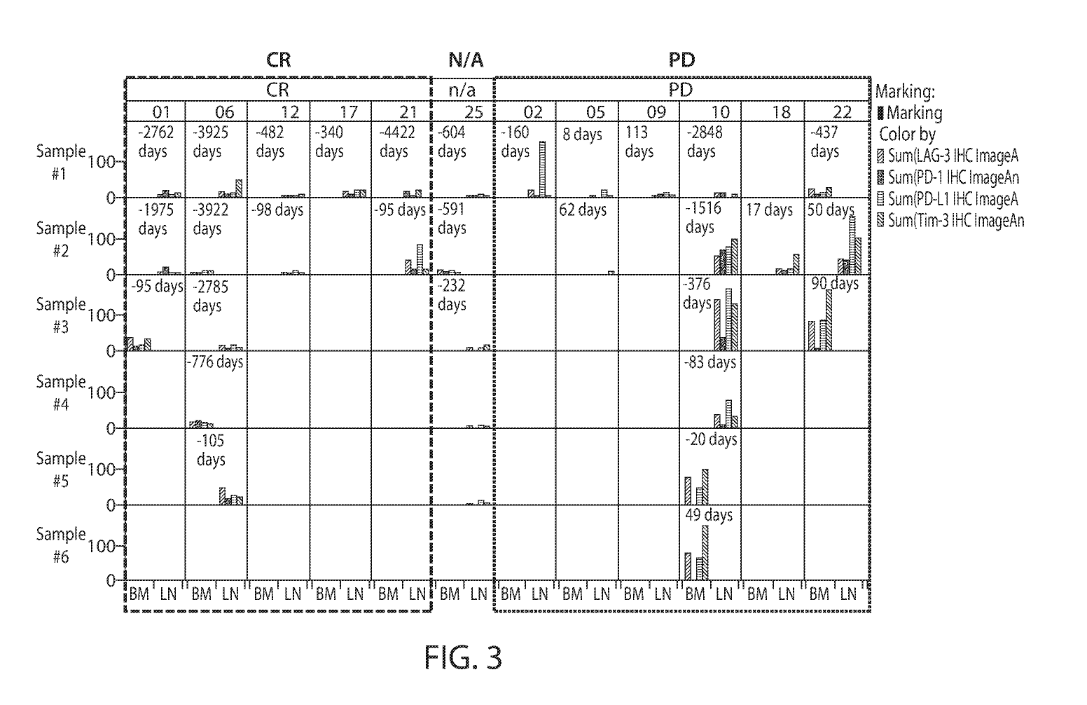

Immune checkpoint protein expression was compared in complete responders (CR) and patients having progressive disease (PD) following treatment with CD19-targeting CAR therapy. As shown in FIG. 3, the CR patients tended to have low levels of PD-L1, PD1, LAG3, and TIM3 before and after treatment, while PD patients tended to have high levels of these molecules before and after treatment. This Example supports combination therapy with a CAR-expressing cell and an immune checkpoint inhibitor, and supports testing to determine immune checkpoint molecule levels in patients receiving a CAR therapy.

### Example 9

### Non-Responder Subset of CLL Patients Exhibit Increased Expression of Immune Checkpoint Inhibitor Molecules

In this study, CART19 cells from clinical manufacture from 34 CLL patients were assessed for expression of immune checkpoint inhibitor molecules, such as PD-1, LAG3, and TIM3. The response of this cohort to CART19 was known and hence a correlation between response and biomarker expression patterns could be assessed.

Manufactured CART19 cells from CLL patients with different responses to CART therapy were analyzed by flow cytometry to determine the expression of CAR and the immune checkpoint inhibitor molecules PD-1, LAG3, and TIM3. The CART19 cells were from: healthy donors (HD) (n=2); CLL patients that responded to CART therapy (CR) (n=5); CLL patients that partially responded to CART therapy (PR) (n=8); CLL patients that did not respond to CART therapy (NR) (n=21). Cells were stained with fluorescently labeled antibodies that specifically recognize CD3, CD4, CD8, CD27, CD45RO, the CAR19 molecule, and immune checkpoint molecules PD-1, LAG3, and TIM3, according to standard methods for flow cytometry analysis known in the art. Expression of each marker, e.g., CD4+, CD8+, etc., was determined by flow cytometry analysis software, and subpopulations (e.g., CD4+ T cells, CD8+ T cells, or CAR19-expressing T cells) were further analyzed for the expression of immune checkpoint molecules PD-1, LAG3, and TIM3.

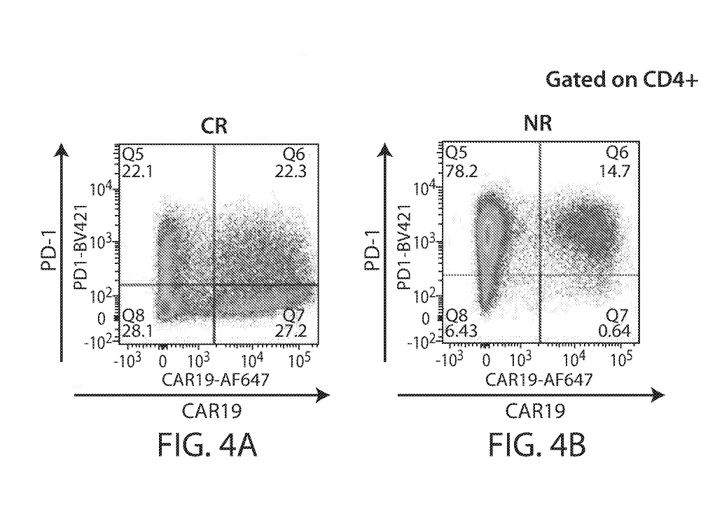

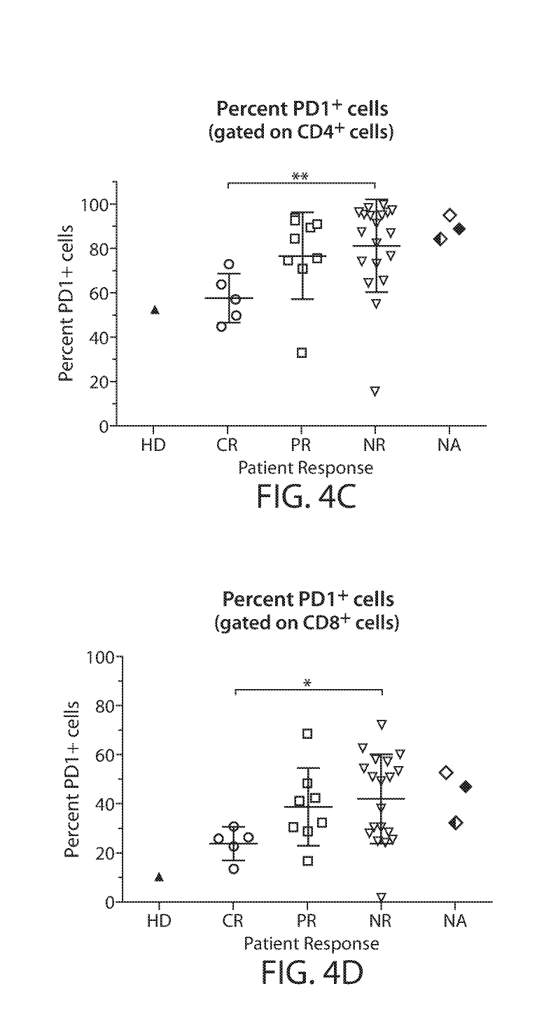

An example of the flow cytometry profiles analysis used to determine surface marker expression is shown in FIGS. 4A and 4B. T cells expressing CD4 were determined using flow cytometry, and were further analyzed for CAR19 and PD-1 expression, such that the x-axis of the profiles indicate CAR19 expression (the top left (Q5) and bottom left (Q8) quadrants show the CAR19-negative CD4+ cells, while the top right (Q6) and bottom right (Q7) quadrants show the CAR19-expressing CD4+ cells) and the y-axis shows PD-1 expression (the bottom left (Q8) and right (Q7) quadrants show the PD-1 negative CD4+ cells and the top left (Q5) and right (Q6) quadrants show the PD-1-expressing CD4+ cells). In the CD4+ population from a CART responder, 44.7% of the CD4+ cells overall expressed PD-1, and about 22.3% of the CAR19-expressing cells were PD-1 positive, while 27.2% of CAR19-expressing cells were PD-1 negative (FIG. 4A). In contrast, in the CD4+ population from a non-responder, there was a significant decrease in CAR19-expressing cells overall (about 15.3% compared to the 49.5% in CR), with 14.7% of the CAR19-expressing cells being PD-1 positive while only 0.64% were PD-1 negative (FIG. 4B). Comparison between the profiles in FIG. 4A and FIG. 4B shows that a much higher percentage of the CD4+ cells from a non-responder express PD-1 (about 92.9%) compared to the CART responder (about 44.7%).

Using the methods and analysis described above, the percentage of PD-1 expressing (PD-1+) cells of the CD4+ population and the CD8+ population was determined for each patient in each response group. Non-responders were shown to have a greater percentage of PD-1+ cells in both the CD4+ (FIG. 4C) and CD8+ (FIG. 4D) populations compared to those that responded to CAR therapy (CR); the increase of average PD-1 percentage was statistically significant for both CD4+ and CD8+ populations. Partial responders (PR) exhibited higher percentages of PD-1+ cells than responders (CR) in both CD4+ (FIG. 4C) and CD8+ (FIG. 4D) populations.

Next, the percentage of PD-1 expressing (PD-1+) cells of the CAR19-expressing CD4+ population and the CAR19-expressing CD8+ population was determined for each patient in each response group. Similar analysis was performed as above, with the additional step of analyzing the CD4+ and CD8+ cells for CAR19-expression, and after identification of the CAR19-expressing cells, determining the percentage of cells with PD-1 expression from the populations of CAR19-expressing cells. A similar trend as that observed in the CD4+ and CD8+ overall populations was observed for the CAR19 expressing CD4+ and CD8+ populations: non-responders were shown to have a greater percentage of PD-1+ cells in both the CD4+ (FIG. 5A) and CD8+ (FIG. 5B) populations compared to those that responded to CAR therapy (CR); the increase of average PD-1 percentage was statistically significant for both CD4+ and CD8+ populations. Partial responders (PR) exhibited higher percentages of PD-1+ cells than responders (CR) in both CD4+ (FIG. 5A) and CD8+ (FIG. 5B) populations.

Further analysis was performed to determine the distribution of cells expressing PD-1, LAG3, and TIM3 from patients with different responses to CAR therapy. Representative cell profile analysis for PD-1, LAG3, and TIM3expression in the CD4+ population is shown in FIG.

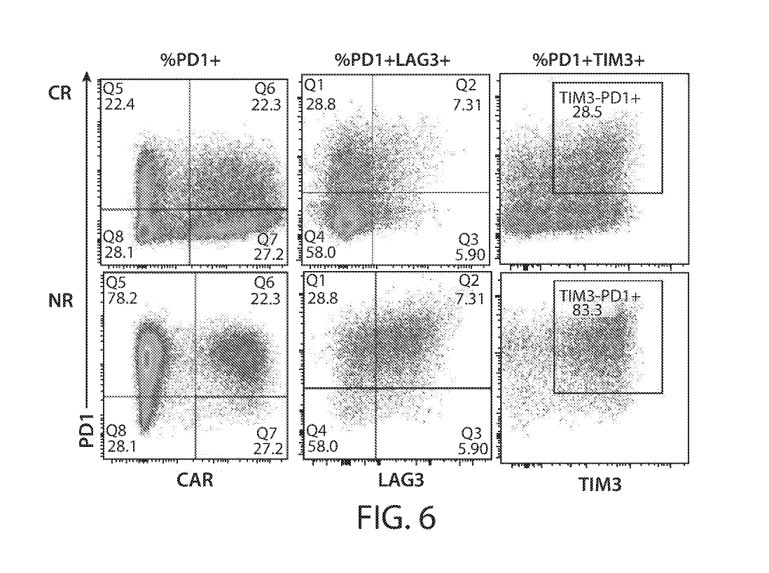

6. The cell populations were first analyzed for CD4+ and CD8+ expression. The CD4+ population (or CD8+ population, not shown) was then analyzed for PD-1 and CAR19 expression (FIG. 6, left profiles). As described previously, non-responders (NR) had a significantly increased percentage of cells that were PD-1+ overall compared to CART responders (CR) (about 92.9% PD-1 positive for NR compared to 44.7% PD-1 positive for CR). Moreover, in non-responders, CAR19-expressing cells were mostly PD-1 positive (14.7% PD-1 positive and CAR+ compared to 0.64% PD-1 negative and CAR+). Then the populations were analyzed for PD-1 and LAG3 co-expression (FIG. 6, middle profiles). Cells that expressed both PD-1 and LAG3 are shown in the top right quadrant (Q2). Non-responders had a significantly increased percentage of cells that expressed both immune checkpoint inhibitors, PD-1 and LAG3, compared to CART responders (67.3% compared to 7.31%). PD-1 expression was also analyzed with TIM3 expression. In FIG. 6, right profiles, the box indicates the cells that express both PD-1 and TIM3. Similar to the results obtained with PD-1 and LAG3, the non-responders had a significantly higher percentage of cells that expressed both immune checkpoint inhibitors, PD-1 and TIM3, compared to CART responders (83.3% compared to 28.5%). The percentage of PD-1 expressing cells (PD1+), PD-1 and LAG3-expressing cells (PD1+LAG3+), and PD-1 and TIM3-expressing cells (PD1+TIM3+) was determined for each patient in each response group using the flow cytometry analysis as described above. Non-responders were shown to have an increased percentage of PD1+ LAG3+ cells (FIG. 7A) and PD1+TIM3+ cells (FIG. 7B) compared to CART responders that was statistically significant for both cell populations. Partial responders also showed an increased percentage of both cell populations compared to CART responders, with the averages being decreased compared to the non-responders.

These results indicate that patients that do not respond to CAR therapy exhibit increased expression of immune checkpoint inhibitors (e.g., PD-1, LAG3, and TIM3) compared to patients that respond or partially respond to CAR therapy. Thus, these results show that agents that inhibit or decrease expression of immune checkpoint inhibitors, e.g., PD-1, LAG3, or TIM3, may be useful for administration to patients receiving CAR therapy to prevent immune suppression through immune checkpoint pathways (e.g., mediated by PD-1, LAG3, or TIM3), thereby increasing the efficacy of the CAR-expressing cells.

### Example 10

### Certain Patients with Primary DLBCL Show CD3+/PD1+ Dual Positive Cancer Cells

Although there have been compelling advances in the cancer immunotherapy space recently in the form of chimeric antigen receptor (CAR) modified T-cells and checkpoint inhibitors, advanced tools to explore the therapeutic mechanisms of their combination are not widely available. To address this growing need, a robust quantitative fluorescent immunohistochemistry platform using multiplex AQUA (Automated Quantitative Analysis) technology was developed to evaluate checkpoint inhibitor expression, enumerate CAR T cells and determine the interaction between tumor cells and immune cells via novel co-localization algorithms. The utility of this method was characterized both in preclinical- and clinical model systems. In an immunodeficient mouse model of B-cell lymphoma, homing of CAR T cells to malignant B-cells in primary lymphoid organs was evaluated. The phenotype and functional status of the CAR T cells via multiplex analyses of CD4, CD8, PD1 and FOXP3 expression was determined. Additionally, to enable combination immunotherapies in Diffuse Large B-Cell Lymphoma (DLBCL) setting, prevalence of adaptive immune resistance mechanisms in the form of PD1 and PD-L1 expression in immune- and tumor cell compartments was examined via landmarks created by cytoplasmic and nuclear stains in both primary and secondary biopsies from DLBCL patients (n=63). To support patient selection for CAR T trials, expression and prevalence of relevant tumor antigens that could not be scored reproducibly by traditional methods were quantified to yield objective cut points. These quantitative multiplexed IHC methods for optimal selection of patients can be utilized in upcoming novel combination immunotherapy trials.

Sample preparation, imaging, and analysis of imaging for DLBCL tissue samples was performed on primary DLBCL (n=49) and secondary DLBCL (15) human patients.

Sample preparation. Formalin fixed paraffin embedded (FFPE) tissue samples were dewaxed. The slides were then rehydrated through a series of xylene to alcohol washes before incubating in distilled water. Heat-induced antigen retrieval was then performed using elevated pressure and temperature conditions, allowed to cool, and transferred to Tris-buffered saline. Staining was then performed where the following steps were carried out. First, endogenous peroxidase was blocked followed by incubation with a protein-blocking solution to reduce nonspecific antibody staining. Next, the slides were stained with a mouse anti-PD1 primary antibody. Slides were then washed before incubation with an anti-mouse HRP secondary antibody. Slides were washed and then PD-1 staining was detected using TSA+Cy® 5 (Perkin Elmer). Primary and secondary antibody reagents were then removed via microwave. The slides were again washed before staining with a rabbit anti-CD3 primary antibody. Slides were washed and then incubated with a cocktail of anti-rabbit HRP secondary antibody plus 4′,6-diamidino-2-phenylindole (DAPI). Slides were washed and then CD3 staining was detected using TSA-Cy® 3 (Perkin Elmer). Slides were washed a final time before they were cover-slipped with mounting media and allowed to dry overnight at room temperature.

Sample imaging and analysis. Fluorescence images were then acquired using the Vectra 2 Intelligent Slide Analysis System using the Vectra software version 2.0.8 (Perkin Elmer). First, monochrome imaging of the slide at 4× magnification using DAPI was conducted. An automated algorithm (developed using inForm) was used to identify areas of the slide containing tissue.

The areas of the slide identified as containing tissue were imaged at 4× magnification for channels associated with DAPI (blue), Cy®3 (green), and Cy® 5 (red) to create RGB images. These 4× magnification images were processed using an automated enrichment algorithm (developed using inForm) in field of view selector to identify and rank possible 20× magnification fields of view according to the highest Cy® 3 expression.

The top 40 fields of view were imaged at 20× magnification across DAPI, Cy®3, and Cy® 5 wavelengths. Raw images were reviewed for acceptability, and images that were out of focus, lacked any tumor cells, were highly necrotic, or contained high levels of fluorescence signal not associated with expected antibody localization (i.e., background staining) were rejected prior to analysis. Accepted images were processed using AQUAduct (Perkin Elmer), wherein each fluorophore was spectrally unmixed by spectral unmixer into individual channels and saved as a separate file.

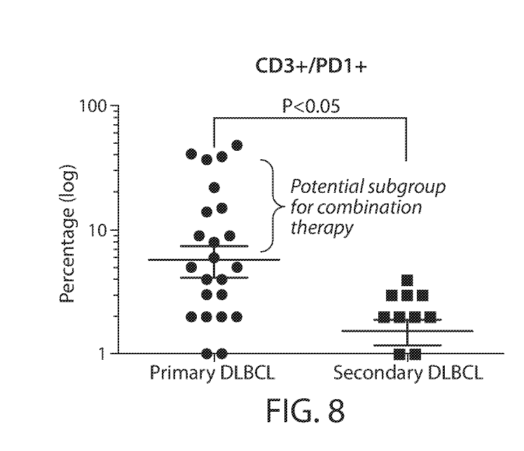

The processed files were further analyzed using AQUAnalysis™ or through a fully automated process using AQUAserve™. Each DAPI image was processed by cell masker to identify all cell nuclei within that image, and then dilated by 2 pixels to represent the approximate size of an entire cell. This resulting mask represented all cells within that image. Each Cy® 5 image was processed by biomarker masker to create a binary mask of all cells that are PD-1-positive. Each Cy® 3 image was processed by biomarker masker to create a binary mask of all cells that are CD3-positive. The binary masks for all cells PD-1-positive and CD3-positive were combined to create a binary mask of all cells that are double positive for PD-1 and CD3. The % biomarker positivity (PBP) for all CD3 cells expressing PD-1 was derived, using positivity calculator, by dividing the total area, measured in pixels and determined by area evaluator, of the mask of all PD-1-positive tumor cells with the total area, measured in pixels and determined by area evaluator, of the mask of all CD3-positive cells. Representative values of PBP for all CD3-positive cells expressing PD-1 in primary and secondary DLBCL human samples are shown in FIG. 8. CD3 and PD-1 status showed that prevalence rates of CD3+/PD-1+ cells in primary is higher than secondary DLBCL setting, providing an opportunity to select patient for either single or combination treatment.

A similar experiment was performed in which PD-L1 was detected using a rabbit anti-PDL1 primary antibody and TSA+Cy5 (Perkin Elmer) on DLBCL tissue samples from primary DLBCL human patients. PD1 and CD3 were also detected on the same samples. The experiment showed that tumor microenvironments comprise cells that express PD1, CD3, and PDL1. The experiment also identified a sub-population of cells that is CD3+PD1+ (data not shown). These results support the model that a tumor microenvironment fosters immune suppressive cells that can be targeted with agents specific to PD1+ or PD-L1+ cells.

### Example 11

### Mutually Exclusive Expression of CD19 and PD-L1 in Samples Comprising DLBCL Cells

Sample preparation. Formalin fixed paraffin embedded (FFPE) tissue samples were dewaxed. The slides were then rehydrated through a series of xylene to alcohol washes before incubating in distilled water. Heat-induced antigen retrieval was then performed using elevated pressure and temperature conditions, allowed to cool, and transferred to Tris-buffered saline. Staining was then performed where the following steps were carried out. First, endogenous peroxidase was blocked followed by incubation with a protein-blocking solution to reduce nonspecific antibody staining. Next, the slides were stained with a rabbit anti-PDL1 primary antibody. Slides were then washed before incubation with an anti-rabbit HRP secondary antibody. Slides were washed and then PDL1 staining was detected using TSA+ Cy® 3 (Perkin Elmer). Primary and secondary antibody reagents were then removed via microwave. The slides were again washed before staining with a mouse anti-CD19 primary antibody. Slides were washed and then incubated with a cocktail of anti-mouse HRP secondary antibody plus 4′,6-diamidino-2-phenylindole (DAPI). Slides were washed and then CD19 staining was detected using TSA-Cy® 5 (Perkin Elmer). Slides were washed a final time before they were cover-slipped with mounting media and allowed to dry overnight at room temperature.

Sample imaging and analysis. Fluorescence images were then acquired using the Vectra 2 Intelligent Slide Analysis System using the Vectra software version 2.0.8 (Perkin Elmer). First, monochrome imaging of the slide at 4× magnification using DAPI was conducted. An automated algorithm (developed using inForm) was used to identify areas of the slide containing tissue.

The areas of the slide identified as containing tissue were imaged at 4× magnification for channels associated with DAPI (blue), Cy®3 (green), and Cy® 5 (red) to create RGB images. These 4× magnification images were processed using an automated enrichment algorithm (developed using inForm) in field of view selector to identify and rank possible 20× magnification fields of view according to the highest Cy® 3 expression.

The top 40 fields of view were imaged at 20× magnification across DAPI, Cy®3, and Cy® 5 wavelengths. Raw images were reviewed for acceptability, and images that were out of focus, lacked any tumor cells, were highly necrotic, or contained high levels of fluorescence signal not associated with expected antibody localization (i.e., background staining) were rejected prior to analysis. Accepted images were processed using AQUAduct (Perkin Elmer), wherein each fluorophore was spectrally unmixed by spectral unmixer into individual channels and saved as a separate file.

The processed files were further analyzed using AQUAnalysis™ or through a fully automated process using AQUAserve™ as described in the previous Example.

Representative values of PBP for all CD19-positive and PD-L1-positive cells in primary and secondary DLBCL human samples are shown in FIG. 9. CD19 and PDL1 expression varied in DLBCL samples. CD19 and PDL1 expression tended to be mutually exclusive, i.e., in general, a given cell expressed CD19 or PD-L1 but not both. While not wishing to be bound by theory, this may be because CD19 is expressed in DLBCL tumor cells while PD-L1 is expressed in non-tumor cells, e.g., cells that support the tumor microenvironment. This observation suggests that a combination therapy of a CD19 inhibitor (e.g., a CD19 CAR-expressing cell) and an inhibitor of PD-L1 signalling may be useful for targeting these two populations of cells.

A similar experiment was performed to, e.g., demonstrate the capability of AQUA analysis to monitor CART19 efficacy. This study monitored CD19, CD3, and the CART19 nucleic acid in samples comprising mixed cells lines with CART19+ Jurkat cells and CD19+ REH cells. CD19 and CD3 proteins were detected by antibodies, and CART19 was detected using an RNA probe against the 3′ UTR of the CAR nucleic acid. The experiment showed that the cell line samples comprise cells that express CD19, CD3, and the CART19 (data not shown). The experiment also showed that the cell line samples comprise a sub-population of cells that is CD3+/CART19+ (data not shown). Proximity analysis was performed, which showed that CART19 cells were physically proximal to CD19+ cells (data not shown). These experiments support the model that CD3+ CART19 cells infiltrate a tumor microenvironment comprising CD19+ cells and physical locations of CD19 and CART19 cells translate into efficacy of the CART19 therapy.

### Example 12

### Pembrolizumab Combined with CD19-Targeted CAR T Cells to Augment Response

Note: Unless otherwise specified, the dose of Pembrolizumab used in this Example was 2 mg/kg based on the patient's weight until a dose of 200 mg was reached, at which point a flat dose of 200 mg was administered.

CD19-targeted chimeric antigen receptor (CAR)-modified T cells have shown complete response (CR) rates exceeding 90% in B-cell acute lymphoblastic leukemia (B-ALL). A subset of patients may not respond to the CAR T therapy or may relapse due to poor CAR T cell persistence. The study described in this Example examined whether inhibition of the PD-1 checkpoint pathway can improve CAR T cell function and persistence.

Patients treated with murine (CTL019) or humanized (CTL119) anti-CD19 CAR T cells received 1-3 doses of the PD-1 inhibitor pembrolizumab starting 14 days-2 months post CAR T cell infusion. Four children with relapsed/refractory B-ALL received pembrolizumab for partial/no response (n=3) or prior history of poor CAR T cell persistence (n=1) after CTL019 (n=1) or CTL119 (n=3) infusion. Pembrolizumab was well tolerated, with fever in 2 patients and no autoimmune toxicity. An increase in detectable circulating CAR+ T cells (% of CD3+ cells by flow cytometry) and/or prolonged detection (compared to prior infusion) was observed in all 4 children after pembrolizumab.

Patients 1 and 2 received CTL119 for CD19+ relapse after prior murine CD19 CAR T cells and were treated with pembrolizumab for partial or no response to CTL119. Both had progressive disease after pembrolizumab, 1 with retained and 1 with decreased CD19 expression.

Two patients had an objective response to the addition of pembrolizumab. In patient 3, prior treatment with both CTL019 and CTL119 resulted in CR with poor CAR T cell persistence followed by CD19+ relapse. After repeat CTL119 infusion combined with pembrolizumab, patient 3 achieved a CR with prolonged CAR T cell persistence (detectable at day 50 compared to loss by day 36 after initial CTL119 infusion). Patient 4, with no prior history of CAR T cell treatment, received pembrolizumab for widespread lymph node involvement at day 28 post CTL019 infusion despite morphologic remission in bone marrow. CAR T cell proliferation after pembrolizumab was associated with dramatic reduction in PET-avid disease by 3 months post CTL019.

The results show that pembrolizumab was safely combined with CAR T cell treatment and increased or prolonged CAR+ T cell detection, with objective responses observed. Thus, immune checkpoint pathways can impact response to CAR T cell treatments.

### Example 13

### Pembrolizumab to Augment Response to CD19 CAR T Cells in Relapsed Acute Lymphoblastic Leukemia (ALL)

Note: Unless otherwise specified, the dose of Pembrolizumab used in this Example was 2 mg/kg based on the patient's weight until a dose of 200 mg was reached, at which point a flat dose of 200 mg was administered.

### Study Design

Relapsed refractory ALL patients previously treated with CD19 CAR-expressing T cells that showed poor persistence of CAR T cells were eligible to receive a repeat infusion of CAR T cells with or without Pembrolizumab. R/R ALL patients were enrolled into a clinical trial (NCT02374333). Patients had chemotherapy and lymphodepletion prior to first infusion of CAR-T cells. On Day −1 a baseline assessment was performed followed by a first infusion of humanized CD19 CAR T cells (CTL119). Patients were assessed on Day 28 for response, and follow-up assessments were performed on months 3, 6, 9 and 12. Patients were monitored for minimal residual disease (MRD), B cell aplasia and CTL119 persistence. Based on the status of CTL119 persistence, patients were re-infused with CTL119. Some patients were also treated with Pembrolizumab at least 2 weeks after re-infusion, or after recovery from CRS. FIG. 10 shows the study design.

### Results

### Case 1: Pembrolizumab for Partial Response

Case 1 describes a patient with R/R ALL with No Response (NR) to prior CD19 CAR therapy. Proliferation of huCART19 was observed in this patient, and on Day 28 the patient presented as a Complete Resposne (CR) with 1.2% CD19+ MRD. At 7 weeks post-infusion, the patient relapsed with CD19+ disease, and low levels of huCART19. The patient was then given Pembrolizumab on Day 52. A modest increase in huCART19 was observed with temporary clearance of peripheral blasts followed by progression of disease.

### Case 2: Pembrolizumab for No Response

Case 2 describes a patient with R/R/ ALL with CD19+ relapse at 12 months post prior CD19 CAR therapy. Good proliferation of huCART19 was observed in this patient. On Day 28, the patient presented as NR with CD19+ relapse. HuCART19 was reinfused into this patient at 6 weeks followed by treatment with Pembrolizumab 14 days after re-infusion. Good proliferation of huCART19 was observed along with prolonged persistence of the cells. At an assessment on Day 28 following re-infusion, the patient showed persistent disease with variable CD19 expression.

### Cases 3-5: Pembrolizumab for Poor Persistence

Cases 3, 4 and 5 describe R/R ALL patients who had prior infusion of huCART19 but showed poor persistence of CAR T cells. These patients had good initial huCART19 proliferation. All 3 patients were given a re-infusion of huCART19 followed by a dose of Pembrolizumab 14 days after the re-infusion.

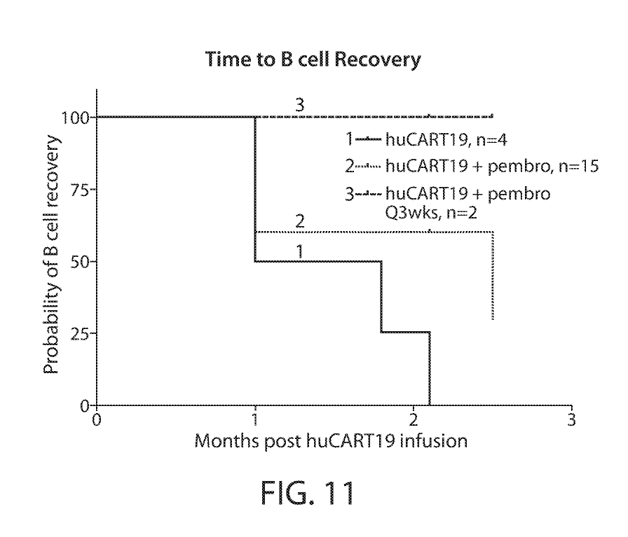

Case 3 describes a patient who had R/R ALL with CD19+ relapse 9 months post prior infusion of huCART19. The Day 28 assessment after the first infusion of huCART19 showed a CR with no MRD detected. Even though the CAR T cells proliferated, the CAR T cells only persisted for a short period with B cell recovery at 2 months. At 15 months post-infusion, the patient had a relapse, and was given a re-infusion of huCART19 at 17 months followed by a dose of Pembrolizumab 14 days later. This patient showed prolonged persistence and continued B cell aplasia with Pembrolizumab administered once every 3 weeks. FIG.11 shows the percentage of huCART19 cells days post huCART19 infusion in the presence or absence of Pembrolizumab treatment. Pembrolizumab increases the persistence of huCART19 cells.

Case 4 describes a patient who presented with R/R ALL with CD19+ relapse 9 months post prior infusion of huCART19. The Day 28 assessment after the first infusion of huCART19 showed a CR with no MRD detected. Even though good CAR T cell proliferation was observed, the CAR T cells only persisted for a short period with B cell recovery observed at 2 months. At 12 months post-infusion, the patient had a relapse and was given a re-infusion of huCART19 at 14 months, followed by a dose of Pembrolizumab 14 days later. No proliferation of huCART19 was observed and the Day 28 assessment after the second infusion revealed No Response (NR) with CD19+ MRD detected.

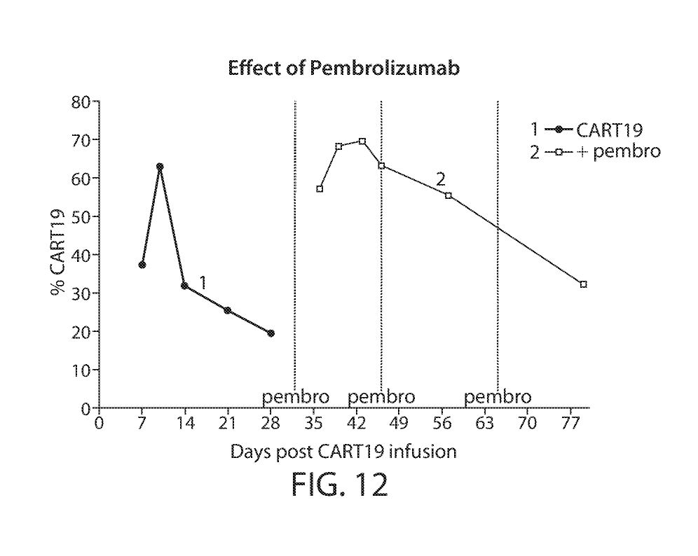

Case 5 describes a patient who presented with R/R ALL with CD19+ relapse 12 months post prior infusion of huCART19. The Day 28 assessment after the first infusion of huCART19 showed a CR with no MRD detected. Even though good CAR T cell proliferation was observed, the CAR T cells only persisted for a short period. 6 months after the first infusion, the patient received a second infusion with short persistence of the CAR T cells. At 8 months after the first infusion, the patient received another infusion of huCART19 followed by a dose of Pembrolizumab 14 days later. This patient showed prolonged persistence and continued B cell aplasia with Pembrolizumab administered once every 3 weeks. FIG. 12 shows a graph comparing the probability of B cell recovery in patients who received only huCART19 (n=4) and patients who received huCART19 and Pembrolizumab (n=7).

### Case 6: Pembrolizumab for Lymphomatous Disease

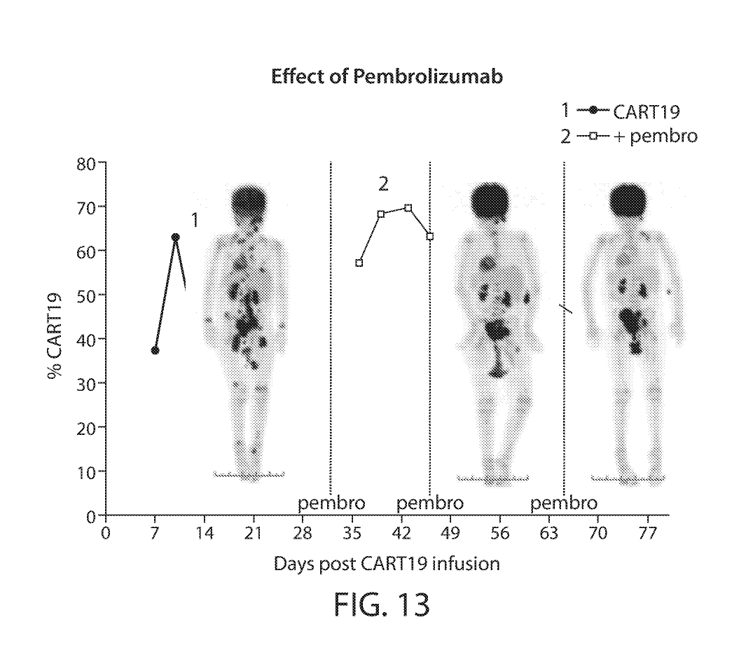

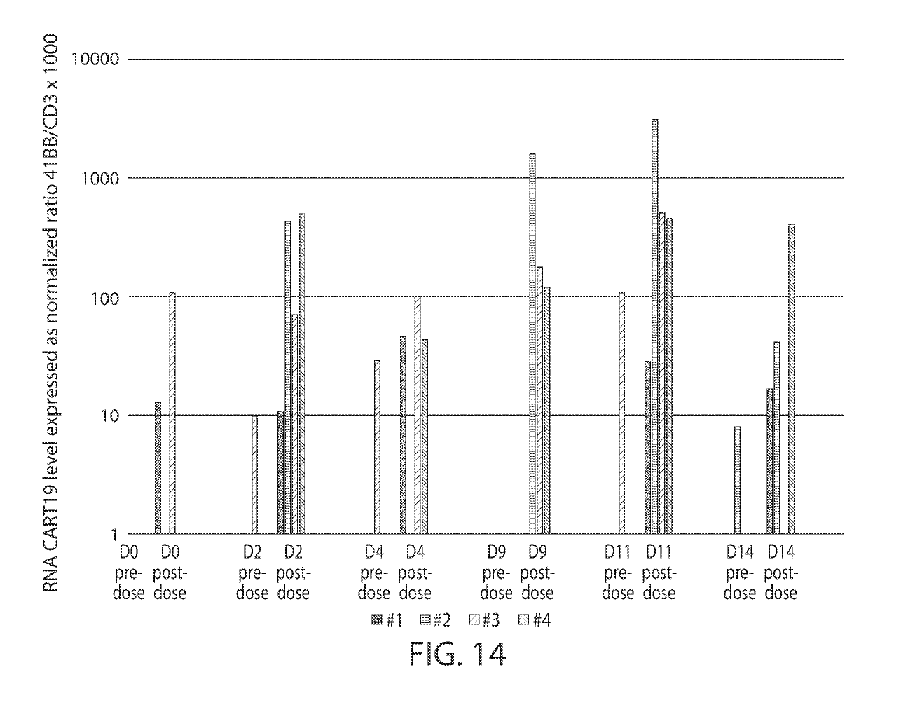

Case 6 describes a patient with R/R ALL with an M3 stage in the bone marrow and widespread lymphomatous disease (LAD). This patient received an infusion of CART19 and the cells proliferated well. The Day 28 assessment showed CR in the bone marrow, however PET analysis showed widespread uptake in the lymph node(s). This patient was then given Pembrolizumab on Day 32 after infusion and once every 2-3 weeks. As shown in FIG. 13, Pembrolizumab treatment increased the percentage of CART19 cells. A decrease in PET avid lesions was also seen after treatment with Pembrolizumab (FIG. 14).

### Example 14

### CD19 Targeted CAR T Cells in Combination with Pembrolizumab in Relapsed/Refractory Diffuse Large B-Cell Lymphoma Patients (r/r DLBCL)

### Study Rationale

CD19 targeted CART therapy (CTL019) is potentially curative in r/r DLBCL in 36-45% of patients. However, PD-L1 is highly expressed on DLBCL cells, resulting in the activation of PD-1 on transduced T cells, e.g.,on CTL019 cells. Activation of PD-1 on CTL019 cells results in functional impairment of the CTL019 therapy. Treatment with anti-PD-1 blocks the PD-1/PD-L1 interaction, which can reactivate CTL019 cells from patients with DLBCL and improve response rates.

An initial analysis of the C2201 (JULIET) study of CTL019 in r/r DLBCL showed that a higher expression of checkpoint inhibitors (e.g., PD-1 and TIM-3) in the CTL019 finished product correlated was observed in patients who were non-responders to the CTL019 therapy compared to responders. Cytokine release syndrome was observed in 57% of patients (57.6 of 99) in this study, of which 11% had Grade 1 CRS, 23% had Grade 2 CRS, 15% had Grade 3 CRS, and 8% had Grade 4 CRS. Among patients who had CRS, the average time (in days) to onset of CRS was 4.1 days with a median of 3.0 days. The earliest patients developed CRS was 1 day after CTL019 administration, and the latest time point at which CRS was observed was 51 days after CTL019 administration. The average duration of CRS in these patients was 8.3 days with a median of 7.0 days, and the range of duration of CRS was 2-30 days in all patients. On average, it took 4.2 days for Grade 3 or Grade 4 CRS to develop. The earliest time point at which Grade 3 or Grade 4 CRS was observed was 2 days, and the latest time point at which Grade 3 or Grade 4 CRS was observed was 8 days.

In the A2101J (DLBCL) study of CTL019 in r/r DLBCL, a higher expression of checkpoint inhibitors (e.g., TIM3, LAG-3, PD1, PD-L1), was observed in biopsy samples, and in CTL019 cells in vivo obtained from non-responders compared to samples obtained from responders. Immunohistochemistry analysis of lymph node and bone marrow samples showed that in patients with progressive disease (PD), higher expression of TIM3, LAG-3, PD1, and PD-L1. Additionally, this study investigating Pembrolizumab in r/r DLBCL showed that 5 out of 9 patients who have progressed after receiving CTL019 responded to treatment with Pembrolizumab. No CRS events were observed in patients who responded to Pembrolizumab, and the duration of response (DoR) was more than 1 year.

Taken together, the data from these trials suggests that anti-PD1 therapy paired with CLT019 can be an effective treatment regimen that provides a potential for cure for patients with r/r DLBCL who are ineligible for a transplant, as demonstrated by higher overall and complete response rates. The combination of anti-PD1 and CTL019 has also shown sustained duration of responses when compared to CTL019 alone and alternative treatment options. The combination therapy has a side effect profile that is similar to CTL019 monotherapy, with no additional long term undesired effects. Therefore, the combination of Pembrolizumab and CTL019 with improved patient outcomes makes it a better, and cost-effective treatment regimen. Additionally, the combination therapy can be administered within a short period of each other, e.g., the anti-PD-1 antibody can be administered soon after (e.g., 5-15 days after) CTL019 administration, e.g., in patients who do not develop CRS. For patients with CRS after CTL019 therapy, the anti-PD-1 antibody can be adminisetered, e.g., upon resolution of CRS.

### Study Design

A Phase I/II study of concurrent administration of CTL019 and Pembrolizumab in r/r (JULIET) DLBCL patient populations will be performed. The single-arm study will enroll 20-25 patients and will include run-in of dose timing findings. Patients with r/r DLBDL who are not eligible for transplant will be enrolled in this study. Five weeks prior to (week −5) commencement of therapy, autologous CTL019 cells will be produced and cryopreserved. Salvage therapy will be initiated during this period and staging of disease will be performed one week prior to (week −1) CTL019 infusion. CTL019 will then be infused into the patients. Pembrolizumab therapy will be given at least 5 days after CTL019 infusion. Six administrations of Pembrolizumab will be given once every 3 weeks at a dose of 300 mg. Patients will be assessed monthly for the first 6 months post-infusion, every 3 months from months 7-24, and every 6 months thereafter. Patients will be followed-up for 15 years per FDA regulations for gene transfer protocols.

The results of this study will guide the initiation of a two-arm randomized Phase II registration study with 90 patients. Patients with r/r DLBCL who are not eligible for transplant will be enrolled into this study. In the Phase II study, one cohort of sixty patients will receive concurrent administration of Pembrolizumab in combination with CTL019, and another cohort of thirty patients will receive administration CTL019 alone. The primary objective of this study will be to evaluate the efficacy of CTL019 in combination with Pembrolizumab. The primary endpoint of this study will be the response rate (RR) of patients at 3 months post-treatment. A secondary objective of this study will be to assess the different in RR at 3 months between patients receiving the combination therapy in comparison with patietns receiving CTL019 alone.

### Example 15

### Pembrolizumab Therapy for Relapsed/Refractory Diffuse Large B-Cell Lymphoma Patients (r/r DLBCL) Previously Treated with CD19 Targeted CAR T Cells

A clinical trial with Pembrolizumab was initiated in patients with r/r DLBCL with documented progression after CTL019 infusion. The first dose of Pembrolizumab was administered as soon as progression was observed and documented. Pembrolizumab is administered once every 3 weeks for 2 years. Patients received Pemrbolizumab around 28 days after CTL019 infusion.

Five out of 9 patients with progressive DLBCL who had previously received CTL019 and were subsequently treated with Pembrolizumab demonstrated a response to the therapy. The longest duration of response was over 1 year. No CRS was observed in these patients.

### Example 16

### Non-Viral, RNA-Redirected Autologous Anti-CD19 T-Cells in Patients with Refractory/Relapsed Hodgkin Lymphoma (HL)

### Background

Cellular therapy using anti-CD19 autologous chimeric antigen receptor T (CART19) cells demonstrated promising outcomes in several hematologic malignancies of B-cell origin, but this therapy has not been studied in Hodgkin Lymphoma (HL) patients. While neoplastic HL Reed-Sternberg (HRS) cells are considered CD19 negative, circulating CD19 positive clonal HRS cell precursors, and CD19 positive reactive cells within the HRS tumor microenvironment represent potential therapeutic targets for CART19 in HL.

### Methods

An open-label pilot study was designed to estimate the feasibility, safety, and efficacy of RNA CART19 cell infusions in patients with relapsed/refractory HL unresponsive to or intolerant of more than one line of standard salvage therapy without curative treatment options. Autologous T-cells electroporated with chimeric anti-CD19 immunoreceptor scFv (RNA CART19 cells) were used in these patients in lieu of more persistent cells engineered by lentiviral transduction, to allow temporal CD19 targeting and limit the window for acute and long term toxicity. Following pheresis and manufacturing of RNA CART19 cells, patients undergo up to six intravenous (IV) infusions of 8 ×10⁵ to 1.5×10⁶ RNA CART19 cells/kg/dose for patients who weighed less than 80kg, and 1×10⁸ RNA CART19 cells/dose (±20%) for patients who weighed more than 80kg. Intravenous cyclophosphamide (30 mg/kg) was administered prior the first and fourth RNA CART19 cell doses to enhance engraftment. Safety and response assessments using Cheson 2007 criteria were measured at defined time points throughout the study. Primary objective was to describe manufacturing feasibility, safety, and biologic engraftment of RNA CART19 cells in relapsed HL. Secondary objectives were to estimate efficacy by overall response rates (ORR) and the effect of RNA CART19 cells on systemic soluble immune factors.

### Results

Five patients were enrolled and had RNA CART19 manufactured, with 4 patients infused and evaluated for response and/or toxicity. The characteristics of the 5 patients at enrollment include: i) a median age of 24 years with the range being 21-42 years; ii) four patients with stage IV/extranodal disease, iii) median number of previous therapies was 5, with the range being 0-8 previous therapies; iv) 4 patients had stem cell transplants (3 patients had autologous stem cell transplants, and one patient had both autologous and allogeneic stem cell transplants). Of the patients treated with the RNA CART19 cells, three pts (60%) had previously progressed on a PD-1 inhibitor. The median absolute lymphocyte count at pheresis was 1,030 mmol/μL (range: 830-2,650). All patients underwent successful manufacturing of RNA CART19. Two patients required bridging chemotherapy, with one patient receiving brentuximab, and the other patient received bendamustine and pembrolizumab. All 4 treated patients underwent lymphodepleting treatment with cyclophosphamide per protocol. The median number of CART19 cells/kg/dose was 1.5×10⁶ (range: 7.3×10⁵ to 1.52×10⁶). Each patient received 6 separate doses or infusions of RNA CART19 cells over a period of 2 weeks. Using qRT-PCR. RNA CART19 was detected transiently in peripheral blood samples immediately post-dose after 80% of the infusions (FIG. 14). There were no study related deaths or grade 3-4 non-hematologic toxicities. Most common grade 1-2 toxicities included transient headache, which was observed in 3 patients and insomnia, which was observed in 2 patients. There was no evidence of cytokine release syndrome. The overall response rate (ORR) at 1 month post-infusion was 50%: One complete resposne (CR) and one partial response (PR) were observed. One additional patient had stable disease (SD). The CR patient progressed at 3 months, and the PR patient was taken off the study to pursue other therapy. The patient with SD progressed at 3 months. Currently, two patients are in CR on a PD-1 inhibitor. One patient is in PR on lenalidomide, and one has died of progressive disease. There have not been any apparent long term toxicities.

### CONCLUSION

These data suggest that cellular therapy using non-viral, RNA-redirected CART19 cells is feasible and safe in patients with relapsed/refractory HL.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A CAR therapy comprising a population of immune effector cells expressing a chimeric antigen receptor (CAR) for use in combination with a PD-1 inhibitor, wherein the CAR comprises an antigen (e.g., a CD19) binding domain, a transmembrane domain and an intracellular signaling domain, and wherein the dose of the PD-1 inhibitor, e.g., anti-PD-1 antibody molecule, is about 200 mg to about 450 mg, e.g., about 300 mg to about 400 mg, e.g., administered every 2 weeks, 3 weeks, 4 weeks, or 5 weeks.

2. A method of treating a subject having a cancer, comprising administering to the subject:
(i) a CAR therapy comprising a population of immune effector cells expressing a chimeric antigen receptor (CAR), wherein the CAR comprises an antigen (e.g., a CD19) binding domain, a transmembrane domain, and an intracellular signaling domain; and
(ii) a PD-1 inhibitor,
wherein the dose of the PD-1 inhibitor, e.g., anti-PD-1 antibody molecule, is about 200 mg to about 450 mg, e.g., about 300 mg to about 400 mg, e.g., administered every 2 weeks, 3 weeks, 4 weeks, or 5 weeks.

3. A CAR therapy comprising a population of immune effector cells expressing a chimeric antigen receptor (CAR) for use in combination with a PD-1 inhibitor, wherein the CAR comprises an antigen (e.g., a CD19) binding domain, a transmembrane domain and an intracellular signaling domain, and wherein administration of the PD-1 inhibitor is initiated 20 days or less after administration of the CAR therapy.

4. A method of treating a subject having a cancer, comprising administering to the subject:
(i) a CAR therapy comprising a population of immune effector cells expressing a chimeric antigen receptor (CAR), wherein the CAR comprises an antigen (e.g., a CD19) binding domain, a transmembrane domain, and an intracellular signaling domain; and
(ii) a PD-1 inhibitor,
wherein administration of the PD-1 inhibitor is initiated 20 days or less after administration of the CAR therapy.

5. The CAR therapy for use or the method of claim 3 or 4, wherein administration of the PD-1 inhibitor is initiated 16 days or less, 15 days or less, 14 days or less, 13 days or less, 12 days or less, 11 days or less, 10 days or less, 9 days or less, 8 days or less, 7 days or less, 6 days or less, 5 days or less, 4 days or less, 3 days or less, 2 days or less, after administration of the CAR therapy.

6. A CAR therapy comprising a population of immune effector cells expressing a chimeric antigen receptor (CAR) for use in combination with a PD-1 inhibitor, wherein the CAR comprises an antigen (e.g., a CD19) binding domain, a transmembrane domain and an intracellular signaling domain, and wherein administration of the PD-1 inhibitor is initiated after the subject has, or is identified as having, one or more of the following:
(a) a partial or no detectable response to the CAR therapy,
(b) a relapsed cancer after the CAR therapy,
(c) a cancer refractory to the CAR therapy;
(d) a progressive form of the cancer after the CAR therapy; or
(e) B cell recovery, e.g., less than 3 months, after the CAR therapy.

7. A method of treating a subject having a cancer, comprising administering to the subject:
(i) a CAR therapy comprising a population of immune effector cells expressing a chimeric antigen receptor (CAR), wherein the CAR comprises an antigen (e.g., a CD19) binding domain, a transmembrane domain, and an intracellular signaling domain; and
(ii) a PD-1 inhibitor,
wherein administration of the PD-1 inhibitor is initiated after the subject has, or is identified as having, one or more of the following:
(a) a partial or no detectable response to the CAR therapy,
(b) a relapsed cancer after the CAR therapy,
(c) a cancer refractory to the CAR therapy; or
(d) a progressive form of the cancer after the CAR therapy or
(e) B cell recovery, e.g., less than 3 months, after the CAR therapy.

8. A CAR therapy comprising a population of immune effector cells expressing a chimeric antigen receptor (CAR) for use in combination with a PD-1 inhibitor, wherein the CAR comprises an antigen (e.g., a CD19) binding domain, a transmembrane domain and an intracellular signaling domain, and wherein administration of the PD-1 inhibitor is initiated after administration of the CAR therapy, and the subject does not have, or has not been identified as having, one or more of the following:
(a) a partial or no detectable response to the CAR therapy,
(b) a relapsed cancer after the CAR therapy,
(c) a cancer refractory to the CAR therapy,
(d) a progressive form of the cancer or
(e) B cell recovery, e.g., less than 3 months, after the CAR therapy.

9. A method of treating a subject having a cancer, comprising administering to the subject:
(i) a CAR therapy comprising a population of immune effector cells expressing a chimeric antigen receptor (CAR), wherein the CAR comprises an antigen (e.g., a CD19) binding domain, a transmembrane domain, and an intracellular signaling domain; and
(ii) a PD-1 inhibitor,
wherein administration of the PD-1 inhibitor is initiated after administration of the CAR therapy, and the subject does not have, or has not been identified as having, one or more of the following:
(a) a partial or no detectable response to the CAR therapy,
(b) a relapsed cancer after the CAR therapy,
(c) a cancer refractory to the CAR therapy,
(d) a progressive form of the cancer, or
(e) B cell recovery, e.g., less than 3 months, after the CAR therapy.

10. The CAR therapy for use or the method of any of the preceding claims, further comprising administering one or more, e.g., 1, 2, 3, 4, or 5 or more, subsequent doses of the PD-1 inhibitor.

11. The CAR therapy for use or the method of claim 10, wherein up to 6 doses of the PD-1 inhibitor are administered.

12. The CAR therapy for use or the method of any of claims 1-11, wherein the method further comprising evaluating the presence or absence of CRS in the subject.

13. The CAR therapy for use or the method of any of claims 1-12, wherein the subject does not have, or is identified, as not having CRS, e.g., severe CRS (e.g., CRS grade 3 or grade 4), after the CAR therapy.

14. The CAR therapy for use or the method of either of claims 12-13, wherein administration of the PD-1 inhibitor is initiated after the subject is identified as not having CRS, e.g., severe CRS (e.g., CRS grade 3 or grade 4), after the CAR therapy.

15. The CAR therapy for use or the method of any of claims 12-14, wherein administration of the PD-1 inhibitor is initiated after treatment of CRS, e.g., after CRS resolution, after the CAR therapy.

16. The CAR therapy for use or the method of any of the preceding claims, wherein the CAR therapy and the PD-1 inhibitor are administered for a treatment interval, and wherein the treatment interval comprises a single dose of the PD-1 inhibitor and a single dose of the CAR-expressing cell.

17. The CAR therapy for use or the method of claim 16, wherein the treatment interval is initiated upon administration of the dose of the CAR-therapy and completed upon administration of the dose of the PD-1 inhibitor.

18. The CAR therapy for use or the method of claim 16 or 17, wherein the treatment interval further comprises administering one or more, e.g., 1, 2, 3, 4, or 5 or more, subsequent doses of the PD-1 inhibitor.

19. The CAR therapy for use or the method of claim 18, wherein up to 6 doses of the PD-1 inhibitor are administered during the treatment interval.

20. The CAR therapy for use or the method of any of claim 1-2 or 6-19, wherein the dose of the CAR-therapy is administered at least 2 days, at least 3 days, at least 4 days, at least 5 days, at least 6 days, at least 7 days, at least 8 days, at least 9 days, at least 10 days, at least 11, days, at least 12, at least 13, at least 14 days, at least 15 days, at least 16 days, at least 17 days, at least 18 days, at least 19 days, or at least 20 days before the dose of PD-1 inhibitor is administered.

21. The CAR therapy for use or the method of claim 20, wherein the dose of the CAR-therapy is administered 25-40 days (e.g., about 25-30, 30-35, or 35-40 days, e.g., about 35 days) before the dose of the PD-1 inhibitor is administered.

22. The CAR therapy for use or the method of any of claim 1-2 or 12-15, wherein the CAR-therapy and the PD-1 inhibitor are administered for a treatment interval, wherein the treatment interval comprises a first and second dose of the PD-1 inhibitor and a dose of the CAR-therapy, and wherein the dose of the CAR-therapy is administered after administration of the first dose of the PD-1 inhibitor but before the administration of the second dose of the PD-1 inhibitor.

23. The CAR therapy for use or the method of claim 22, wherein the treatment interval is initiated upon administration of the first dose of the PD-1 inhibitor and completed upon administration of the second dose of the PD-1 inhibitor.

24. The CAR therapy for use or the method of claim 22 or 23, wherein the second dose of the PD-1 inhibitor is administered at least 5 days, 7 days, 1 week, 2 weeks, or 3 weeks after administration of the first dose of the PD-1 inhibitor.

25. The CAR therapy for use or the method of any of claims 22-24, wherein the dose of the CAR-therapy is administered at least 2 days, 3 days, 4 days, 5 days, 6 days, 7 days, or 2 weeks after administration of the first dose of the PD-1 inhibitor.

26. The CAR therapy for use or the method of any of claims 22-25, wherein the second dose of the PD-1 inhibitor is administered at least 2 days, 3 days, 4 days, 5 days, 6 days, 7 days, or 2 weeks after administration of the dose of the CAR-therapy.

27. The CAR therapy for use or the method of any of claims 16-26, wherein the treatment interval is repeated, e.g., one or more times, e.g., 1, 2, 3, 4, or 5 more times.

28. The CAR therapy for use or the method of any of claims 16-27, wherein the treatment interval is followed by one or more, e.g., 1, 2, 3, 4, or 5, subsequent treatment intervals.

29. The CAR therapy for use or the method of claim 28, wherein the one or more subsequent treatment interval is different from the first or previous treatment interval.

30. The CAR therapy for use or the method of claim 28 or 29, wherein the one or more subsequent treatment intervals is administered at least 1 day, e.g., at least 1 day, at least 2 days, at least 3 days, at least 4 days, at least 5 days, at least 6 days, at least 7 days, at least 2 weeks, at least 1 month, at least 3 months, at least 6 months, or at least 1 year after the completion of the first or previous treatment interval.

31. The CAR therapy for use or the method of any of claims 16-30, wherein one or more subsequent doses, e.g., 1, 2, 3, 4, or 5 or more doses, of the PD-1 inhibitor is administered after the completion of one or more treatment intervals.

32. The CAR therapy for use or the method of any of claims 16-31, wherein a dose of the PD-1 inhibitor is administered every 5 days, 6 days, 7 days, 10 days, 2 weeks, 3 weeks, or 4 weeks after the completion of one or more treatment intervals.

33. The CAR therapy for use or the method of any of claims 16-32, wherein the treatment interval comprises a dose of CAR-therapy administered 2-20 days, 5-17 days, 7-16 days, 8-16 days, 10-15 days, 14-21 days or 2-3 weeks before the dose of the PD-1 inhibitor is administered, and wherein the treatment interval is repeated 0-52 times, and wherein the treatment intervals are initiated at least 1 day, at least 2 days, at least 3 days, at least 4 days, at least 5 days, at least 6 days, at least 7 days, at least 2 weeks, at least 1 month, at least 3 months, at least 6 months, or at least 1 year after the completion of the previous treatment interval.

34. The CAR therapy for use or the method of claim 33, wherein one or more subsequent doses of the PD-1 inhibitor is administered every 5 days, 7 days, 2 weeks, 3 weeks, or 4 weeks, after the second treatment interval.

35. The CAR therapy for use or the method of any of claims 1-15, wherein the subject is administered a single dose of a CAR-expressing cell and a single dose of a PD-1 inhibitor.

36. The CAR therapy for use or the method of claim 35, wherein the single dose of the CAR-expressing cell is administered at least 2 days, e.g., 2, 3, 4, 5, 6, 7, 8, 9, 10, 11, 12, 13, 14, 15, 16, 17, 18, 19 or 20 days, before administration of the single dose of the PD-1 inhibitor.

37. The CAR therapy for use or the method of claim 35 or 36, wherein the CAR-therapy comprises an RNA CAR molecule, e.g., an in vitro transcribed (IVT) RNA, and wherein one or more, e.g., 1, 2, 3, 4, or 5, subsequent doses of a CAR-therapy is administered to the subject after the initial dose of the CAR-therapy.

38. The CAR therapy for use or the method of claim 37, wherein the one or more subsequent doses of the CAR-expressing cell are administered at least 2 days, e.g., 2, 3, 4, 5, 6, 7 days, 2 weeks, or 3 weeks, after the previous dose of the CAR-expressing cell.

39. The CAR therapy for use or the method of any of claims 27-38, wherein one or more, e.g., 1, 2, 3, 4, or 5, or more subsequent doses of PD-1 inhibitor are administered after administration of the single dose of the PD-1 inhibitor.

40. The CAR therapy for use or the method of claim 39, wherein the one or more subsequent doses of the PD-1 inhibitor are administered at least 5 days, 7 days, 2 weeks, 3 weeks or 4 weeks, after the previous dose of PD-1 inhibitor.

41. The CAR therapy for use or the method of claim 39 or 40, wherein the one or more subsequent doses of the PD-1 inhibitor are administered at least 1, 2, 3, 4, 5, 6, or 7 days, or 2 weeks or 3 weeks, after a dose of the CAR-therapy, e.g., the initial dose of the CAR-therapy.

42. The CAR therapy for use or the method of any of claims 27-41, wherein the administration of the one or more doses of the CAR-expressing cell and the one or more doses of PD-1 inhibitor is repeated.

43. The CAR therapy for use or the method of any of the preceding claims, wherein the CAR therapy comprises a dose of CAR-expressing cells comprising about 10⁴ to about 10⁹ cells/kg, e.g., about 10⁴ to about 10⁵ cells/kg, about 10⁵ to about 10⁶ cells/kg, about 10⁶ to about 10⁷ cells/kg, about 10⁷ to about 10⁸ cells/kg, about 10⁸ to about 10⁹ cells/kg, or about 1-5×10⁷ cells/kg to about 1-5 ×10⁸ cells/kg.

44. The CAR therapy for use or the method claim 43, wherein the dose of CAR-expressing cells is about 1-5 ×10⁷ cells/kg.

45. The CAR therapy for use or the method of claim 43, wherein the dose of CAR-expressing cells is about 1-5 ×10⁸ cells/kg.

46. The CAR therapy for use or the method of any of claims 3-45, wherein the dose of the PD-1 inhibitor is between 1 and 30 mg/kg, e.g., about 1 to 25 mg/kg, about 2 to 20 mg/kg, about 2 to 5 mg/kg, or about 2 mg/kg, about 3 mg/kg, about 4 mg/kg, or about 5 mg/kg.

47. The CAR therapy for use or the method of claim 46, wherein the dose of the PD-1 inhibitor is about 1 to 20 mg/kg, or about 2-5 mg/kg e.g., administered every 2 weeks, 3 weeks, 4 weeks, or 5 weeks .

48. The CAR therapy for use or the method of any of claims 1-45, wherein the dose of the PD-1 inhibitor, e.g., anti-PD-1 antibody molecule, is about 200 mg to about 450 mg, e.g., about 200 mg to about 400 mg, e.g., administered every 2 weeks, 3 weeks, 4 weeks, or 5 weeks.

49. The CAR therapy for use or the method of any of claims 1-48, wherein the dose of the PD-1 inhibitor is about 200 mg or about 300 mg, e.g., administered every 3 weeks, e.g., via intravenous infusion.

50. The CAR therapy for use or the method of any of claims 1-48, wherein the dose of the PD-1 inhibitor is about 400 mg, e.g., administered every 4 weeks, e.g., via intravenous infusion.

51. The CAR therapy for use or the method of any of claims 1-48, wherein the PD-1 inhibitor is a PD-1 antibody molecule and is administered at a dose of about 300 mg every 2 weeks, 3 weeks, or 4 weeks, and the CAR therapy is administered at a dose of 1-5×10⁸ cells.

52. The CAR therapy for use or the method of any of the preceding claims, wherein the PD-1 inhibitor comprises an antibody molecule, a small molecule, a polypeptide, e.g., a fusion protein, or an inhibitory nucleic acid, e.g., a siRNA or shRNA.

53. The CAR therapy for use or the method of any of the preceding claims, wherein the PD-1 inhibitor is characterized by one or more of the following:
a. inhibits or reduces PD-1 expression, e.g., transcription or translation of PD-1;
b. inhibits or reduces PD-1 activity, e.g., inhibits or reduces binding of PD-1 to its cognate ligand, e.g., PD-L1 or PD-L2; or
c. binds to PD-1 or its ligand(s), e.g., PD-L1 or PD-L2.

54. The CAR therapy for use or the method of any of the preceding claims, wherein the PD-1 inhibitor is an antibody molecule.

55. The CAR therapy for use or the method of the preceding claims, wherein the PD-1 inhibitor is selected from the group consisting of Nivolumab, Pembrolizumab, PDR001, Pidilizumab, AMP 514, AMP-224, and any anti-PD-1 antibody molecule provided in Table 6.

56. The CAR therapy for use or the method of any of the preceding claims, wherein the PD-1 inhibitor comprises an anti-PD-1 antibody molecule comprising
a. a heavy chain complementary determining region 1 (HC CDR1), a heavy chain complementary determining region 2 (HC CDR2), and a heavy chain complementary determining region 3 (HC CDR3) of any PD-1 antibody molecule amino acid sequence listed in Table 6; and
b. a light chain complementary determining region 1 (LC CDR1), a light chain complementary determining region 2 (LC CDR2), and a light chain complementary determining region 3 (LC CDR3) of any PD-1 antibody molecule amino acid sequence listed in Table 6.

57. The CAR therapy for use or the method of claim 56 wherein the anti-PD-1 antibody molecule thereof comprises
a) a HC CDR1 amino acid sequence chosen from SEQ ID NO: 137 or 140, a HC CDR2 amino acid sequence of SEQ ID NO: 138 or 141, and a HC CDR3 amino acid sequence of SEQ ID NO: 139; and
b) a LC CDR1 amino acid sequence of SEQ ID NO: 146 or 149, a LC CDR2 amino acid sequence of SEQ ID NO: 147 or 150, and a LC CDR3 amino acid sequence of SEQ ID NO: 148, 151, 166, or 167 (e.g., a LC CDR3 amino acid sequence of SEQ ID NO: 166 or 167).

58. The CAR therapy for use or the method of claim 56 or 57, wherein the anti-PD-1 antibody molecule comprises a heavy chain variable region comprising:
i) the amino acid sequence of any heavy chain variable region listed in Table 6, e.g., SEQ ID NOs: 142, 144, 154, 158, 172, 184, 216, or 220;
ii) the amino acid sequence having at least one, two or three modifications but not more than 30, 20 or 10 modifications to the amino acid sequence of any heavy chain variable region provided in Table 6, e.g., SEQ ID NOs: 142, 144, 154, 158, 172, 184, 216, or 220; or
iii) an amino acid sequence with 95-99% identity to the amino acid sequence of any heavy chain variable region provided in Table 6, e.g., SEQ ID NOs: 142, 144, 154, 158, 172, 184, 216, or 220.

59. The CAR therapy for use or the method of any of claims 56-58, wherein the anti-PD-1 antibody molecule comprises a heavy chain comprising:
i) the amino acid sequence of any heavy chain listed in Table 6, e.g., SEQ ID NOs: 156, 160, 174, 186, 218, 222, 225, or 236;
ii) the amino acid sequence having at least one, two or three modifications but not more than 30, 20 or 10 modifications to any heavy chain listed in Table 6, e.g., SEQ ID NOs: 156, 160, 174, 186, 218, 222, 225, or 236; or
iii) an amino acid sequence with 95-99% identity to the amino acid sequence of any heavy chain listed in Table 6, e.g., SEQ ID NOs: 156, 160, 174, 186, 218, 222, 225, or 236.

60. The CAR therapy for use or the method of any of claims 56-59, wherein the anti-PD-1 antibody molecule comprises a light chain variable region comprising:
i) the amino acid sequence of any light chain variable region listed in Table 6, e.g., SEQ ID NOs: 152, 162, 168, 176, 180, 188, 192, 196, 200, 204, 208, or 212;
ii) the amino acid sequence having at least one, two or three modifications but not more than 30, 20 or 10 modifications to the amino acid sequence of any light chain variable region provided in Table 6, e.g., SEQ ID NOs: 152, 162, 168, 176, 180, 188, 192, 196, 200, 204, 208, or 212; or
iii) an amino acid sequence with 95-99% identity to the amino acid sequence of any light chain variable region provided in Table 6, e.g., SEQ ID NOs: 152, 162, 168, 176, 180, 188, 192, 196, 200, 204, 208, or 212.

61. The CAR therapy for use or the method of any of claims 56-60, wherein the anti-PD-1 antibody molecule comprises a light chain comprising:
i) the amino acid sequence of any light chain listed in Table 6, e.g., SEQ ID NOs: 164, 170, 178, 182, 190, 194, 198, 202, 206, 210, or 214;
ii) the amino acid sequence having at least one, two or three modifications but not more than 30, 20 or 10 modifications to any light chain listed in Table 6, e.g., SEQ ID NOs: 164, 170, 178, 182, 190, 194, 198, 202, 206, 210, or 214; or
iii) an amino acid sequence with 95-99% identity to the amino acid sequence to any any light chain listed in Table 6, e.g., SEQ ID NOs: 164, 170, 178, 182, 190, 194, 198, 202, 206, 210, or 214.

62. The CAR therapy for use or the method of any of claims 56-61, wherein the anti-PD-1 antibody molecule comprises:
i) a heavy chain variable domain comprising the amino acid sequence of SEQ ID NO: 172 and a light chain variable domain comprising the amino acid sequence of SEQ ID NO: 204
ii) a heavy chain variable domain comprising the amino acid sequence of SEQ ID NO: 142 or 144 and a light chain variable domain comprising the amino acid sequence of SEQ ID NO: 152;
iii) a heavy chain variable domain comprising the amino acid sequence of SEQ ID NO: 154 or 158 and a light chain variable domain comprising the amino acid sequence of SEQ ID NO: 162;
iv) a heavy chain variable domain comprising the amino acid sequence of SEQ ID NO: 154 or 158 and a light chain variable domain comprising the amino acid sequence of SEQ ID NO: 168;
v) a heavy chain variable domain comprising the amino acid sequence of SEQ ID NO: 172 and a light chain variable domain comprising the amino acid sequence of SEQ ID NO: 176;
vi) a heavy chain variable domain comprising the amino acid sequence of SEQ ID NO: 172 and a light chain variable domain comprising the amino acid sequence of SEQ ID NO: 180;
vii) a heavy chain variable domain comprising the amino acid sequence of SEQ ID NO: 184 and a light chain variable domain comprising the amino acid sequence of SEQ ID NO: 180;
viii) a heavy chain variable domain comprising the amino acid sequence of SEQ ID NO: 184 and a light chain variable domain comprising the amino acid sequence of SEQ ID NO: 188;
ix)a heavy chain variable domain comprising the amino acid sequence of SEQ ID NO: 172 and a light chain variable domain comprising the amino acid sequence of SEQ ID NO: 188;
x) a heavy chain variable domain comprising the amino acid sequence of SEQ ID NO: 172 and a light chain variable domain comprising the amino acid sequence of SEQ ID NO: 192;
xi) a heavy chain variable domain comprising the amino acid sequence of SEQ ID NO: 172 and a light chain variable domain comprising the amino acid sequence of SEQ ID NO: 196;
xii) a heavy chain variable domain comprising the amino acid sequence of SEQ ID NO: 184 and a light chain variable domain comprising the amino acid sequence of SEQ ID NO: 200;
xiii) a heavy chain variable domain comprising the amino acid sequence of SEQ ID NO: 172 and a light chain variable domain comprising the amino acid sequence of SEQ ID NO: 200;
xiv) a heavy chain variable domain comprising the amino acid sequence of SEQ ID NO: 184 and a light chain variable domain comprising the amino acid sequence of SEQ ID NO: 204;
xv) a heavy chain variable domain comprising the amino acid sequence of SEQ ID NO: 172 and a light chain variable domain comprising the amino acid sequence of SEQ ID NO: 204;
xvi) a heavy chain variable domain comprising the amino acid sequence of SEQ ID NO: 172 and a light chain variable domain comprising the amino acid sequence of SEQ ID NO: 208;
xvii) a heavy chain variable domain comprising the amino acid sequence of SEQ ID NO: 172 and a light chain variable domain comprising the amino acid sequence of SEQ ID NO: 212;
xviii) a heavy chain variable domain comprising the amino acid sequence of SEQ ID NO: 216 and a light chain variable domain comprising the amino acid sequence of SEQ ID NO: 204;
xix) a heavy chain variable domain comprising the amino acid sequence of SEQ ID NO: 216 and a light chain variable domain comprising the amino acid sequence of SEQ ID NO: 200;
xx) a heavy chain variable domain comprising the amino acid sequence of SEQ ID NO: 220 and a light chain variable domain comprising the amino acid sequence of SEQ ID NO: 200;
xxi) a heavy chain variable domain comprising the amino acid sequence of SEQ ID NO: 172 and a light chain variable domain comprising the amino acid sequence of SEQ ID NO: 176;
xxii) a heavy chain variable domain comprising the amino acid sequence of SEQ ID NO: 172 and a light chain variable domain comprising the amino acid sequence of SEQ ID NO: 188;
xxiii) a heavy chain variable domain comprising the amino acid sequence of SEQ ID NO: 172 and a light chain variable domain comprising the amino acid sequence of SEQ ID NO: 200; or
xxiv) a heavy chain variable domain comprising the amino acid sequence of SEQ ID NO: 184 and a light chain variable domain comprising the amino acid sequence of SEQ ID NO: 204.

63. The CAR therapy for use or the method of any of claims 56-62, wherein the anti-PD-1 antibody molecule comprises:
i) a heavy chain comprising the amino acid sequence of SEQ ID NO: 225 and a light chain comprising the amino acid sequence of SEQ ID NO: 206;
ii) a heavy chain comprising the amino acid sequence of SEQ ID NO: 144 and a light chain comprising the amino acid sequence of SEQ ID NO: 152;
iii) a heavy chain comprising the amino acid sequence of SEQ ID NO: 156 or 160 and a light chain comprising the amino acid sequence of SEQ ID NO: 164;
iv) a heavy chain comprising the amino acid sequence of SEQ ID NO: 156 or 160 and a light chain comprising the amino acid sequence of SEQ ID NO: 170.
v) a heavy chain comprising the amino acid sequence of SEQ ID NO: 174 and a light chain comprising the amino acid sequence of SEQ ID NO: 178;
vi) a heavy chain comprising the amino acid sequence of SEQ ID NO: 174 and a light chain comprising the amino acid sequence of SEQ ID NO: 182;
vii) a heavy chain comprising the amino acid sequence of SEQ ID NO: 186 and a light chain comprising the amino acid sequence of SEQ ID NO: 182;
viii) a heavy chain comprising the amino acid sequence of SEQ ID NO: 186 and a light chain comprising the amino acid sequence of SEQ ID NO: 190;
ix) a heavy chain comprising the amino acid sequence of SEQ ID NO: 174 and a light chain comprising the amino acid sequence of SEQ ID NO: 190;
x) a heavy chain comprising the amino acid sequence of SEQ ID NO: 174 and a light chain comprising the amino acid sequence of SEQ ID NO: 194;
xi) a heavy chain comprising the amino acid sequence of SEQ ID NO: 174 and a light chain comprising the amino acid sequence of SEQ ID NO: 198;
xii) a heavy chain comprising the amino acid sequence of SEQ ID NO: 186 and a light chain comprising the amino acid sequence of SEQ ID NO: 202;
xiii) a heavy chain comprising the amino acid sequence of SEQ ID NO: 174 and a light chain comprising the amino acid sequence of SEQ ID NO: 202;
xiv) a heavy chain comprising the amino acid sequence of SEQ ID NO: 186 and a light chain comprising the amino acid sequence of SEQ ID NO: 206;
xv) a heavy chain comprising the amino acid sequence of SEQ ID NO: 174 and a light chain comprising the amino acid sequence of SEQ ID NO: 206;
xvi) a heavy chain comprising the amino acid sequence of SEQ ID NO: 174 and a light chain comprising the amino acid sequence of SEQ ID NO: 210;
xvii) a heavy chain comprising the amino acid sequence of SEQ ID NO: 174 and a light chain comprising the amino acid sequence of SEQ ID NO: 214;
xviii) a heavy chain comprising the amino acid sequence of SEQ ID NO: 218 and a light chain comprising the amino acid sequence of SEQ ID NO: 206;
xix) a heavy chain comprising the amino acid sequence of SEQ ID NO: 218 and a light chain comprising the amino acid sequence of SEQ ID NO: 202;
xx) a heavy chain comprising the amino acid sequence of SEQ ID NO: 222 and a light chain comprising the amino acid sequence of SEQ ID NO: 202;
xxi) a heavy chain comprising the amino acid sequence of SEQ ID NO: 225 and a light chain comprising the amino acid sequence of SEQ ID NO: 178;
xxii) a heavy chain comprising the amino acid sequence of SEQ ID NO: 225 and a light chain comprising the amino acid sequence of SEQ ID NO: 190;
xxiii) a heavy chain comprising the amino acid sequence of SEQ ID NO: 225 and a light chain comprising the amino acid sequence of SEQ ID NO: 202; or
xxiv) a heavy chain comprising the amino acid sequence of SEQ ID NO: 236 and a light chain comprising the amino acid sequence of SEQ ID NO: 206.

64. The CAR therapy for use or the method of any of claims 56-63, wherein the PD-1 inhibitor comprises an anti-PD-1 antibody molecule comprising a heavy chain variable domain comprising the amino acid sequence of SEQ ID NO: 172 and a light chain variable domain comprising the amino acid sequence of SEQ ID NO: 204.

65. The CAR therapy for use or the method of claim 64, wherein the anti-PD1 antibody molecule comprises:
(i) a heavy chain variable (VH) region comprising the VHCDR1 amino acid sequence of SEQ ID NO: 503; the VHCDR2 amino acid sequence of SEQ ID NO: 504; and the VHCDR3 amino acid sequence of SEQ ID NO: 505; and
(ii) a light chain variable (VL) region comprising the VLCDR1 amino acid sequence of SEQ ID NO: 500; the VLCDR2 amino acid sequence of SEQ ID NO: 501; and rge VLCDR3 amino acid sequence of SEQ ID NO: 502,
or an amino acid sequence at least 85%, 90%, 95% identical or higher.

66. The CAR therapy for use or the method of any of the preceding claims, wherein the CD19 binding domain comprises a heavy chain complementary determining region 1 (HC CDR1), a heavy chain complementary determining region 2 (HC CDR2), and a heavy chain complementary determining region 3 (HC CDR3) of any CD19 heavy chain binding domain amino acid sequence listed in Table 2 or 3; and a light chain complementary determining region 1 (LC CDR1), a light chain complementary determining region 2 (LC CDR2), and a light chain complementary determining region 3 (LC CDR3) of any CD19 light chain binding domain amino acid sequence listed in Table 2 or 3.

67. The CAR therapy for use or the method of claim 66, wherein the CD19 binding domain comprises a HC CDR1, a HC CDR2, and a HC CDR3 according to the HC CDR amino acid sequences in Table 4, and a LC CDR1, a LC CDR2, and a LC CDR3 according to the LC CDR amino acid sequences in Table 5.

68. The CAR therapy for use or the method of any of the preceding claims, wherein the CD19 binding domain comprises:
a. the amino acid sequence of any heavy chain variable region of a CD19 binding domain listed in Table 2 or 3;
b. an amino acid sequence having at least one, two or three modifications but not more than 30, 20 or 10 modifications to the amino acid sequence of any heavy chain variable region of a CD19 binding domain provided in Table 2 or 3; or
c. an amino acid sequence at least 95% identical, e.g., with 95-99% identity, to the amino acid sequence of any heavy chain variable region of a CD19 binding domain provided in Table 2 or 3.

69. The CAR therapy for use or the method of any of the preceding claims, wherein the CD19 binding domain comprises:
a. the amino acid sequence of any heavy chain of a CD19 binding domain provided in Table 2 or 3;
b. an amino acid sequence having at least one, two or three modifications but not more than 30, 20 or 10 modifications to any heavy chain of a CD19 binding domain provided in Table 2 or 3; or
c. an amino acid sequence at least 95% identical, e.g., with 95-99% identity to the amino acid sequence to any heavy chain of a CD19 binding domain provided in Table 2 or 3.

70. The CAR therapy for use or the method of any of the preceding claims, wherein the CD19 binding domain comprises:
a. the amino acid sequence of any light chain variable region of a CD19 binding domain provided in Table 2 or 3;
b. an amino acid sequence having at least one, two or three modifications but not more than 30, 20 or 10 modifications to the amino acid sequence of any light chain variable region of a CD19 binding domain provided in Table 2 or 3; or
c. an amino acid sequence at least 95% identical, e.g., with 95-99% identity to the amino acid sequence of any light chain variable region of a CD19 binding domain provided in Table 2 or 3.

71. The CAR therapy for use or the method of any of the preceding claims, wherein the CD19 binding domain comprises:
a. the amino acid sequence of any light chain of a CD19 binding domain provided in Table 2 or 3;
b. the amino acid sequence having at least one, two or three modifications but not more than 30, 20 or 10 modifications to any light chain of a CD19 binding domain provided in Table 2 or 3; or
c. an amino acid sequence at least 95% identical, e.g., with 95-99%identity to the amino acid sequence to any light chain of a CD19 binding domain provided in Table 2 or 3.

72. The CAR therapy for use or the method of any of the preceding claims, wherein the CD19 binding domain comprises the amino acid sequence of any heavy chain variable region listed in Table 2 or 3, and the amino acid sequence of any light chain variable region listed in Table 2 or 3.

73. The CAR therapy for use or the method of any of the preceding claims, wherein the CD19 binding domain comprises:
a. the amino acid sequence selected from the group consisting of SEQ ID NO: 109, SEQ ID NO: 45, SEQ ID NO: 46, SEQ ID NO: 47, SEQ ID NO:48, SEQ ID NO:49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 110, SEQ ID NO: 112, or SEQ ID NO: 115;
b. an amino acid sequence having at least one, two or three modifications but not more than 30, 20 or 10 modifications to any of SEQ ID NO: 109, SEQ ID NO: 45, SEQ ID NO: 46, SEQ ID NO: 47, SEQ ID NO:48, SEQ ID NO:49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 110, SEQ ID NO: 112, or SEQ ID NO: 115; or
c. an amino acid sequence at least 95% identical, e.g., with 95-99%identity to the amino acid sequence to any of SEQ ID NO: 109, SEQ ID NO: 45, SEQ ID NO: 46, SEQ ID NO: 47, SEQ ID NO:48, SEQ ID NO:49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 110, SEQ ID NO: 112, or SEQ ID NO: 115.

74. The CAR therapy for use or the method of any of the preceding claims, wherein the transmembrane domain comprises a transmembrane domain from a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154.

75. The CAR therapy for use or the method of any of the preceding claims, wherein the transmembrane domain comprises
(i) the amino acid sequence of SEQ ID NO: 6,
(ii) an amino acid sequence comprises at least one, two or three modifications but not more than 20, 10 or 5 modifications of the amino acid sequence of SEQ ID NO:6, or
(iii) a sequence at least 95% identical, e.g., with 95-99% identity, to the amino acid sequence of SEQ ID NO:6.

76. The CAR therapy for use or the method of any of the preceding claims, wherein the CD19 binding domain is connected to the transmembrane domain by a hinge region.

77. The CAR therapy for use or the method of any of the preceding claims, wherein the hinge region comprises SEQ ID NO:2, or a sequence at least 95% identical, e.g., with 95-99%, identity thereof.

78. The CAR therapy for use or the method of any of the preceding claims, wherein the intracellular signaling domain comprises a costimulatory signaling domain comprising a functional signaling domain obtained from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

79. The CAR therapy for use or the method of claim 59, wherein the costimulatory domain comprises the amino acid sequence of SEQ ID NO:7, or an amino acid sequence having at least one, two or three modifications but not more than 20, 10 or 5 modifications of the amino acid sequence of SEQ ID NO:7, or an amino acid sequence at least 95% identical to the amino acid sequence of SEQ ID NO:7.

80. The CAR therapy for use or the method of any of the preceding claims, wherein the intracellular signaling domain comprises a functional signaling domain of 4-1BB and/or a functional signaling domain of CD3 zeta.

81. The CAR therapy for use or the method of any of the preceding claims, wherein the intracellular signaling domain comprises the amino acid sequence of SEQ ID NO: 7 and/or the amino acid sequence of SEQ ID NO:9 or SEQ ID NO:10; or an amino acid sequence having at least one, two or three modifications but not more than 20, 10 or 5 modifications of the amino acid sequence of SEQ ID NO:7 and/or the amino acid sequence of SEQ ID NO:9 or SEQ ID NO:10; or an amino acid sequence at least 95% identical to the amino acid sequence of SEQ ID NO:7 and/or the amino acid sequence of SEQ ID NO:9 or SEQ ID NO:10.

82. The CAR therapy for use or the method of any of the preceding claims, wherein the intracellular signaling domain comprises the amino acid sequence of SEQ ID NO:7 and the amino acid sequence of SEQ ID NO:9 or SEQ ID NO:10, wherein the amino acid sequences comprising the intracellular signaling domain are expressed in the same frame and as a single polypeptide chain.

83. The CAR therapy for use or the method of any of the preceding claims, wherein the CAR further comprises a leader sequence comprising the amino acid sequence of SEQ ID NO:1.

84. The CAR therapy for use or the method of any of the preceding claims, wherein the CAR comprises:
(i) the amino acid sequence of any of SEQ ID NO: 108; SEQ ID NO: 93; SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 111, SEQ ID NO: 114, or SEQ ID NO: 116;
(ii) an amino acid sequence having at least one, two or three modifications but not more than 30, 20 or 10 modifications to any of SEQ ID NO: 108; SEQ ID NO: 93; SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 111, SEQ ID NO: 114, or SEQ ID NO: 116; or
(iii) an amino acid sequence at least 95 identical to any of SEQ ID NO: 108; SEQ ID NO: 93; SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 103, SEQ ID NO: 104, SEQ ID NO: 111, SEQ ID NO: 114, or SEQ ID NO: 116.

85. The CAR therapy for use or the method of any of the preceding claims, wherein the cell comprising a CAR comprises a nucleic acid encoding the CAR.

86. The CAR therapy for use or the method of claim 85, wherein the nucleic acid encoding the CAR is a lentiviral vector.

87. The CAR therapy for use or the method of claim 85 or 86, wherein the nucleic acid encoding the CAR is introduced into the cells by lentiviral transduction.

88. The CAR therapy for use or the method of any of claims 85-87, wherein the nucleic acid encoding the CAR is an RNA, e.g., an in vitro transcribed RNA.

89. The CAR therapy for use or the method of claim 88, wherein the nucleic acid encoding the CAR is introduced into the cells by electroporation.

90. The CAR therapy for use or the method of any of the preceding claims, wherein the cell is a T cell or an NK cell.

91. The CAR therapy for use or the method of claim 90, wherein the T cell is an autologous or allogeneic T cell.

92. The CAR therapy for use or the method of any of the preceding claims, further comprising administering an additional anti-cancer agent.

93. The CAR therapy for use or the method of any of the preceding claims, wherein the cancer is a hematological cancer.

94. The CAR therapy for use or the method of any of the preceding claims, wherein the cancer is a lymphoma or a leukemia.

95. The CAR therapy for use or the method of claim 93, wherein the cancer is chosen from one or more of B-cell acute lymphoid leukemia (BALL), T-cell acute lymphoid leukemia (TALL), small lymphocytic leukemia (SLL), acute lymphoid leukemia (ALL), chronic myelogenous leukemia (CML), chronic lymphocytic leukemia (CLL), mantle cell lymphoma (MCL), B cell prolymphocytic leukemia, blastic plasmacytoid dendritic cell neoplasm, Burkitt's lymphoma, diffuse large B cell lymphoma (DLBCL), follicular lymphoma, hairy cell leukemia, small cell- or a large cell-follicular lymphoma, malignant lymphoproliferative conditions, MALT lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, or Waldenstrom macroglobulinemia.

96. The CAR therapy for use or the method of claim 93, wherein the cancer is acute lymphoid leukemia (ALL), e.g., prediatric B-ALL, or a B cell lymphoma, e.g., pediatric B cell lymphoma.

97. The CAR therapy for use or the method of claim 93, wherein the cancer is diffuse large B cell lymphoma (DLBCL), e.g., relapsed or refractory DLBCL.

98. The CAR therapy for use or the method of any of the preceding claims, wherein the subject is a mammal, e.g., a human.

99. The CAR therapy for use or the method of any of the preceding claims, wherein the subject expresses PD-1, PD-L1 and/or PD-L2.

100. The CAR therapy for use or the method of claim 99, wherein a cancer cell or a cell in close proximity to a cancer cell in the subject expresses PD-1, PD-L1, and/or PD-L2.

101. The CAR therapy for use or the method of claim 99 or 100, wherein the cancer cell is from a DLBCL sample, e.g., from a relapsed or refractory DLBCL sample.

102. The CAR therapy for use or the method of any of the preceding claims, wherein the cell expressing a CAR expresses PD-1, PD-L1, and/or PD-L2.

103. The CAR therapy for use or the method of any of claims 1-102, wherein the subject has, or is identified as having, a higher number or percentage of immune effector cells, e.g., CD4⁺ and/or CD8⁺ T cells, expressing one, two, three, or all of PD-1, LAG-3 or TIM-3, compared to a reference value, e.g., a complete responder to the CAR therapy.

104. The CAR therapy for use or the method of 103, wherein the subject has, or is identified as having, a higher number of: PD-1 expressing immune effector cells, e.g., CD4⁺ and/or CD8⁺ T cells; PD-1 and LAG-3-expressing immune effector cells, e.g., CD4⁺ and/or CD8⁺ T cells; PD-1 and TIM-3 expressing immune effector cells, e.g., CD4⁺ and/or CD8⁺ T cells; or PD-1, TIM-3 and LAG-3 expressing immune effector cells, e.g., CD4⁺ and/or CD8⁺ T cells.

105. The CAR therapy for use or the method of 103 or 104, wherein the immune effector cells, e.g., CD4⁺ and/or CD8⁺ T cells, coexpress a CAR, e.g., a CD19 CAR.

106. A combination comprising:
a cell, e.g., a population of immune effector cells, comprising a CAR, wherein the CAR comprises an antigen binding domain, a transmembrane domain, and an intracellular signaling domain; and
a PD-1 inhibitor chosen pembrolizumab, nivolumab, or any of the antibody molecules from Table 6, e.g., comprising the variable light chain and the variable heavy chain amino acid sequences of SEQ ID NO: 204 and SEQ ID NO: 172,
for use in treating a cancer, in a subject.

107. A composition (e.g., one or more compositions or dosage forms), comprising:
a cell, e.g., a population of immune effector cells, comprising a CAR, wherein the CAR comprises an antigen binding domain, a transmembrane domain, and an intracellular signaling domain, and
a PD-1 inhibitor chosen from Table 6, e.g., comprising the variable light chain and the variable heavy chain amino acid sequences of SEQ ID NO: 204 and SEQ ID NO: 172.

108. The method, combination, or composition of any of the preceding claims, wherein the CD19 binding domain is the amino acid sequence of SEQ ID NO: 109; or wherein the CAR comprises the amino acid sequence of SEQ ID NO: 108.
